SEQUENCE LISTING (3 sequences)

SEQ ID NO: 1 - protein, 14 aa, Artificial Sequence
  Chemically synthesized
ILSRRPSYRKILND

SEQ ID NO: 2 - protein, 10 aa, Artificial Sequence
  MOD_RES 6: Phosphorylated serine
GSRRPSYRKI

SEQ ID NO: 3 - protein, 11 aa, Artificial Sequence
  AMIDATION 11: Amido c-terminus
GLYRSPSMPEN

(19) United States
(12) Patent Application Publication
(10) Pub. No.: US 2005/0245563 A1
(43) Pub. Date: Nov. 3, 2005

(54) Chk-1 inhibitors

(75) Inventors: Robert George Boyle, Cambridge (GB); Hassan Julien Imogai, Annemasse (FR); Michael Cherry, Newmarket (GB); Alfred John Humphries, Saffron Walden (GB); Eva Figueroa Navarro, Cambridge (GB); David Rodney Owen, Cambridge (GB); Natalie A. Dales, Arlington, MA (US); Matthew J. LaMarche, Reading, MA (US); Courtney Cullis, Bedford, MA (US); Alexandra E. Gould, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Millennium Pharmaceuticals, Inc., Cambridge, MA (US)

(21) Appl. No.: 10/857,620

(22) Filed: May 28, 2004

Related U.S. Application Data
(60) Provisional application No. 60/474,161, filed on May 29, 2003.

(51) Int. Cl. C07D 471/14; A61K 31/4745

(52) U.S. Cl. 514/292; 546/82

(57) ABSTRACT

Disclosed are novel inhibitors of Chk-1 and methods of using the same for therapy.

### RELATED APPLICATION

This application claims the benefit of U.S. Provisional Application No. 60/474,161, filed on May 29, 2003. The entire teachings of the above application are incorporated herein by reference.

### BACKGROUND OF THE INVENTION

Cell cycle checkpoints are regulatory pathways that control the order and timing of cell cycle transitions. They ensure that critical events such as DNA replication and chromosome segregation are completed in high fidelity. The regulation of these cell cycle checkpoints is a critical determinant of the manner in which tumor cells respond to many chemotherapies and radiation. Many effective cancer therapies work by causing DNA damage; however, resistance to these agents remains a significant limitation in the treatment of cancer. There are several mechanisms of drug resistance: an important one is attributed to the prevention of cell cycle progression through the control of critical activation of a checkpoint pathway that arrests the cell cycle to provide time for repair and induces the transcription of genes to facilitate repair, thereby avoiding immediate cell death. By abrogating checkpoint arrests at, for example, the G2 checkpoint, it may be possible to synergistically augment tumor cell death induced by DNA damage and circumvent resistance. (Shyan et al., U.S. Pat. No. 6,723,498 (2004)). Human Chk-1 plays a role in regulating cell cycle arrest by phosphorylating the phosphatase cdc25 on Serine 216, which may be involved in preventing activation of cdc2/cyclin B and initiating mitosis. (Sanchez et al., Science, 277:1497 (1997)). Therefore, inhibition of Chk-1 should enhance DNA damaging agents by initiating mitosis before DNA repair is complete and thereby causing tumor cell death.

### SUMMARY OF THE INVENTION

It has now been found that certain 2,5-dihydro-pyrazolo[4,3-c]quinolin-4-ones are effective inhibitors of Chk-1. For example, the compounds as described in Example 126 have an IC₅₀ less than 20 μM when tested in an in vitro assay that assesses the inhibitory activity of test compounds. Based on these discoveries, novel Chk-1 inhibitors, methods of inhibiting Chk-1 in a subject and methods of treating cancer are disclosed herein.

One embodiment of the present invention is a Chk-1 inhibitor represented by Structural Formula (I):

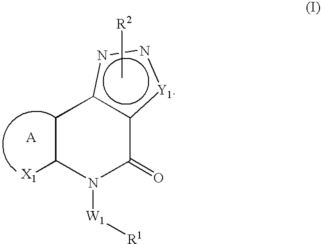

Ring A is a monocyclic aromatic group that is optionally substituted at any one or more substitutable ring atoms and is optionally fused to a second monocyclic aromatic group, Ring B.

Ring B is optionally substituted at any one or more substitutable ring atoms.

Y₁ is N or CR³.

R₁ is —H, —CONR¹¹R¹², —COOR¹², an optionally substituted heteroaryl group, an optionally substituted non-aromatic heterocyclic group, and W₁ is a linear C1-C6 alkylidene chain. R₁ is —OR¹², —NR¹¹R¹², —CN, —NR¹¹CONR¹¹R¹², —NR¹¹COR¹², —NH—C(═NR¹¹)NR¹¹R¹², —SO₂NR¹¹R¹², —NR¹¹SO₂R¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR₁₁R¹², —OC(O)—C(R^{12c}R^{12c})—OR¹², OC(O)—C(R^{12c}R^{12c})—NR¹¹R¹², cycloalkyl or -Ph and W₁ is a linear C2-C6 alkylidene group; or —W₁—R₁ is —H.

An additional value for R₁ when W₁ is a linear C1-C6 alkylidene chain includes —C(═NR¹¹)—NR¹¹R¹². Additional values for R¹ when W₁ is a linear C2-C6 alkylidene group include —O—C(O)—OR¹², —N═C(NR¹¹R¹²)₂, —NR¹¹CO—(CH₂)ₙCH(NR^{12a}R^{12a})—R¹², —NR₁₁—C(R¹²)—C(O)OR¹², —NR₁₁—C(R¹²)—C(O)NR¹¹R¹² and —NR¹¹—C(R¹²)CH₂OR¹². The alkylidene group represented by W₁ is optionally monosubstituted with —OR^{12b}, —N(R^{12b})₂, or a spiro cycloalkyl group. Additionally, W₁ is optionally monosubstituted with oxo or halo. Additionally, the alkylidene group represented by W₁ is optionally substituted with one or more —CH₃ groups. Additionally, the alkylidene group represented by W₁ is monosubstituted with —OR^{12b} or —N(R^{12b})₂ when R¹ is cycloalkyl or -Ph. Preferably, the alkylidene group represented by W₁ is optionally monosubstituted with —OR^{12b} or —N(R^{12b})₂ and/or is optionally substituted with one or more —CH₃ groups, provided that the alkylidene group represented by W₁ is monosubstituted with —OR^{12b} or —N(R^{12b})₂ when R₁ is cycloalkyl or -Ph.

R² is —H or a group that is cleavable in vivo.

R³ is —H, halogen, alkyl, haloalkyl or —V₁—R^{3a}. V₁ is a covalent bond or a C1-C4 alkylidene optionally substituted with one or more methyl groups or with a spiro cycloalkyl group. Additionally, V₁ is a C1-C4 alkylidene optionally substituted with one or more —OR^{a}, —NR^{b}R^{c}, alkyl, hydroxyalkyl, alkoxyalkyl, aminoalkyl groups. R^{3a} is —OR^{a}, —SR^{a}, —CONR^{b}R^{c}, —NR^{b}R^{c}, —NHC(O)NR^{a}R^{b}, —CN, —COOH, —COOR^{a}, —NHC(O)H, —NHC(O)R^{a}, —OC(O)R^{a}, —OC(O)NR^{b}R^{c}, —NHC(O)—OR^{a}, boronate, alkyl boronate, or an optionally substituted aromatic or aralkyl group. Additional values of R^{3a} include —S(O)₂NR^{b}R^{c}, —S(O)₂(R^{a}), —C(═NR^{a})—NR^{b}R^{c}, —NH—C(═NR^{a})NR^{b}R^{c}, —NH—C(═NR^{a})R^{a}, or an optionally substituted non-aromatic cycloaliphatic or heterocyclic group. R^{a} is —H, alkyl or an optionally substituted aromatic or aralkyl group; and R^{b} and R^{c} are independently —H, alkyl or an optionally substituted aromatic or aralkyl group; or —NR^{b}R^{c} is an optionally substituted nitrogen-containing non-aromatic heterocyclic group.

X₁ is O, S, N, or CR⁴ when R¹ is —CONR¹¹R¹², —COOR¹², —C(═NR¹¹)—NR¹¹R¹², an optionally substituted heteroaryl group, an optionally substituted non-aromatic heterocyclic group, and W₁ is a linear C1-C6 alkylidene chain; R₁ is —OR¹², —NR¹¹R¹², —CN, —NR¹¹CONR¹¹R¹², —NR¹¹COR¹², —NH—C(═NR₁₁)NR¹¹R¹², —N═C(NR¹¹R¹²)₂, —SO₂NR¹¹R¹², —NR¹¹SO₂R¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —NR¹¹CO—(CH₂)ₙCH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR₁₁R¹², —NR¹¹—C(R¹²)—C(O)OR¹², —NR¹¹—C(R¹²)—C(O)NR₁₁R¹², —NR₁₁—C(R¹²)CH₂OR¹², cycloalkyl or -Ph; and X₁ is C—W₂—R⁵ when R₁ is —H and when —W₁—R¹ is —H.

W₂ is a linear C1-C6 alkylidene chain, optionally monosubstituted with —OR^{12b}, —N(R^{12b})₂, or a spiro cycloalkyl group or with one or more —CH₃ groups. Additionally, the C1-C6 alkylidene group represented by W₂ optionally has a cyclopropyl group, a monomethylated cyclopropyl group or dimethylated cyclopropyl group fused thereto and one carbon atom in the C1-C6 alkylidene group represented by W₂ is optionally replaced with T. Preferably, W₂ is -T-W₃, wherein W₃ is a linear C2-C5 alkylidene chain, optionally monosubstituted with —OR^{12b}, —N(R^{12b})₂, or a spiro cycloalkyl group and/or optionally substituted with one or more —CH₃ groups, and additionally, the alkylidene chain represented by W₃ optionally has a cyclopropyl, monomethylated cyclopropyl or dimethylated dimethylated cyclopropyl group fused thereto.

T is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—. An additional value for T includes —C≡C—. Preferably, T is a covalent bond or —O—.

R⁴ is —H, C1-C3 alkyl, C1-C3 haloalkyl, halogen, hydroxy, C1-C3 alkoxy, C1-C3 haloalkoxy, —NH₂, C1-C3 alkylamine, C1-C3 dialkylamine, —NHC(O)H, —NHC(O)(C1-C3 alkyl), —C(O)NH₂, —C(O)NH(C1-C3 alkyl) or —C(O)N(C1-C3 alkyl)₂.

R⁴ is an optionally substituted heteroaryl group, an optionally substituted non-aromatic heterocyclic group,—OR¹², —NR¹¹R¹², —CN, —NR¹¹CONR¹¹R¹², —NR¹¹SO₂R¹², —NR¹¹COR¹², —NH—C(═NR¹¹)NR¹¹R¹²,—SO₂NR¹¹R¹², —CONR¹¹R¹², —COOR¹², —OC(O)R¹², —NR¹¹C(O)OR¹², —OC(O)—NR¹¹R¹², —NR¹¹CO—CH(OR^{12a})—R¹², —NR₁₁CO—CH(NR^{12a}R^{12a})—R¹², —OC(O)—CH(OR^{12a})—R₁₂, —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —CH(NR¹¹R¹²)-Ph, —CH(NR¹¹R¹²)-(cycloalkyl), a cycloalkyl group or a phenyl group substituted with —V₂—OR¹² or —V—NR¹¹R¹². V₂ is a covalent bond or a C1-C5 alkylene group.

R⁶ is —H or C1-C3 alkyl.

Each R¹¹ is independently —H or a C1-C3 alkyl group.

Each R¹² is independently —H, an optionally substituted alkyl group, aromatic group, aralkyl group, non-aromatic heterocyclic group or non-aromatic heterocyclylalkyl; or —NR¹¹R¹² is an optionally substituted non-aromatic nitrogen-containing heterocyclic group.

Each R^{12a} is independently —H, a C1-C3 alkyl group, —C(O)H, —C(O)—(C1-C3 alkyl), —C(O)NH₂, —C(O)NH—(C1-C3 alkyl), —C(O)N—(C1-C3 alkyl)₂, —C(O)O—(C1-C3 alkyl), —S(O)₂(C1-C3 alkyl) or —NR^{12a}R^{12a} taken together is a substituted or unsubstituted non-aromatic nitrogen-containing heterocyclic group. Preferably, each R^{12a} is independently —H or —CH₃ or —NR^{12a}R^{12a} taken together is an aziridinyl group.

Each R^{12b} is independently —H or a C1-C3 alkyl group or —NR^{12b}R^{12b} taken together is a substituted or unsubstituted non-aromatic nitrogen-containing heterocyclic group.

Each R^{12c} is independently —H, a C1-C3 alkyl group or —C(R^{12c}R^{12c})— taken together is a C3-C8 cycloalkyl group. Preferably, each R^{12a} is independently —H or —CH₃ or —C(R^{12c}R^{12c})— taken together is a cyclopropyl group.

Ph is an optionally substituted phenyl group.

n is an integer from 1 to 4. Preferably n is an integer from 1 to 2. More preferably n is 1.

Another embodiment of the present invention is a method of treating cancer in a subject. The method comprises administering to the subject an effective amount of the Chk-1 inhibitor represented by Structural Formula (I).

Yet another embodiment of the present invention is a method of inhibiting Chk-1 in a subject in need of such treatment. The method comprises administering to the subject an effective amount of a Chk-1 inhibitor disclosed herein.

Yet another embodiment of the present invention is a method of treating a proliferative disorder in a subject comprising administering an effective amount of a Chk-1 inhibitor disclosed herein.

Yet another embodiment of the present invention is a method of inhibiting Chk-1 in a cell in a subject in need of such treatment by contacting the cell with an effective amount of a Chk-1 inhibitor disclosed herein.

Yet another embodiment of the present invention is a method of inhibiting Chk-1 in a cell in vitro by contacting the cell with an effective amount of a Chk-1 inhibitor disclosed herein.

Yet another embodiment of the present invention is a pharmaceutical composition comprising a Chk-1 inhibitor disclosed herein and a pharmaceutically effective excipient, carrier or diluent. The pharmaceutical compositions can be used in therapy, e.g., to inhibit Chk-1 activity in a subject in need of such inhibition or to treat a subject with cancer.

Yet another embodiment of the present invention is the use of a Chk-1 inhibitor disclosed herein for the manufacture of a medicament for inhibiting Chk-1 in a subject in need of such inhibition or for treating a subject with cancer.

The compounds disclosed herein are effective inhibitors of Chk-1. They are therefore expected to be effective in treating subjects with cancer and enhancing the effectiveness of many current anti-cancer therapies, including radiation therapy and anti-cancer agents that exert their cytotoxic activity by damaging the genetic material of cancer cells and inhibiting cellular replication. In addition, the disclosed Chk-1 inhibitors, when used in combination with current anti-cancer therapies are expected to be effective against multidrug resistant cancers.

### DETAILED DESCRIPTION OF THE INVENTION

The present invention is directed to Chk-1 inhibitors represented by Structural Formula (I) and to novel methods of therapy utilizing the Chk-1 inhibitors represented by Structural Formula (I).

In a preferred embodiment, the disclosed Chk-1 inhibitor is represented by Structural Formula (II):

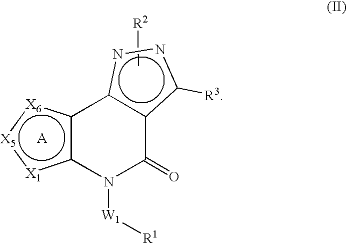

In Structural Formula (II), X₁ is S and X₁, X₅ and X₆, taken together are —S—CH═CH—; X₁ is S and X₁, X₅ and X₆, taken together are —S—CH═N—; X₁ is O and X₁, X₅ and X₆, taken together are —O—CH═CH—; X₁ is O and X₁, X₅ and X₆, taken together are —O—CH═N—; X₁ is NH and X₁, X₅ and X₆, taken together are —NH—CH═CH—; X₁ is NH and X₁, X₅ and X₆, taken together are —NH—CH═N—; X₁ is NH and X₁, X₅ and X₆, taken together are —NH—N═CH—; X₁ is CH and X₁, X₅ and X₆, taken together are —CH═CH—S—; X₁ is CH and X₁, X₅ and X₆, taken together are —CH═CH—O—; X₁ is CH and X₁, X₅ and X₆, taken together are —CH═CH—NH—; X₁ is CH and X₁, X₅ and X₆, taken together are —CH═N—O—; X₁ is CH and X₁, X₅ and X₆, taken together are —CH═N—S—; X₁ is CH and X₁, X₅ and X₆, taken together are —CH═N—NH—; X₁ is CH and X₁, X₅ and X₆, taken together are —CH═N—CH—; X₁ is O and X₁, X₅ and X₆, taken together are —O—N═CH—; X₁ is S and X₁, X₅ and X₆, taken together are —S—N═C—; X₁ is N and X₁, X₅ and X₆, taken together are —N═CH—S—; X₁ is N and X₁, X₅ and X₆, taken together are —N═CH—O—, provided that Ring A is optionally substituted at any one or more substitutable ring carbon atoms and provided that Ring A is optionally fused to a phenyl ring, Ring E, that is optionally substituted at any one or more substitutable ring carbon atoms.

The remainder of the variables in Structural Formula (II) are as defined above for Structural Formula (I).

In another preferred embodiment, the disclosed Chk-1 inhibitor is represented by Structural Formulas (I) or (IV):

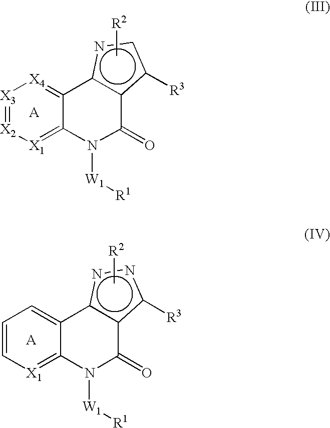

In Structural Formula (III) and (IV), X₁ is N, or CR⁴.

In Structural Formula (III), X₂, X₃ and X₄ are independently N or CH, provided that Ring A in Structural Formula (m) is not a tetrazole or a 1,2,3-triazole, provided that Ring A in Structural Formula (III) and in Structural Formula (IV) is optionally substituted at any one or more substitutable ring carbon atoms and provided that Ring A in Structural Formula (III) and in Structural Formula (IV) is optionally fused to a phenyl ring, Ring C, that is optionally substituted at any one or more substitutable ring carbon atoms.

The remainder of the variables in Structural Formulas (III) and (IV) are as defined above for Structural Formula (I).

In another preferred embodiment, the disclosed Chk-1 inhibitors are represented by Structural Formulas (V) or (VI):

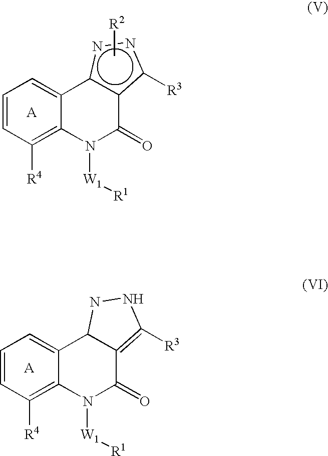

Ring A in Structural Formulas (V) and (VI) is optionally substituted at any one or more substitutable ring carbon atoms.

R³, V₁ and R^{3a} are as described above for Structural Formula (I) but preferably R³ is methyl, ethyl, cyclopropyl, cyclopentyl, or tetrahydrofuryl, or R³ is V₁—R^{3a}, wherein V₁ is a C1-C2 alkylidene and R^{3a} is —OH or —OCH₃.

The remainder of the variables in Structural Formulas (V) and (VI) are as provided above for Structural Formula (I).

One preferred set of values for the variables in Structural Formulas (V) and (VI) is described below in the following six paragraphs.

R¹ is —OR¹², —NR¹¹R¹², —CN, an optionally substituted nitrogen-containing heteroaryl group, an optionally substituted non-aromatic nitrogen-containing heterocyclic group, —NHCOR¹², —OC(O)R¹², —NHC(O)NR¹¹R¹², —OC(O)NR¹¹R¹², or —NHC(O)OR¹². An additional value for R₁ when W₁ is a linear C2-C6 alkylidene group is —O—C(O)—OR¹². Alternatively, R₁ is —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR₁₁CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—N¹¹R¹² —NHCO—CH(OH)—R¹², —NHCO—CH(NH₂)—R¹², —CH(OH)—CONR¹¹R¹², —CH(NH₂)—CONR², —OC(O)—CH(OH)—R¹², or —OC(O)—CH(N₂)—R¹².

W₁ is C2-C6 alkylene, —(CH₂)ₚ—CH(R²⁰)—CH₂—, —(CH₂)ₚ—C(R²¹)₂—CH₂— or —(CH₂)ₚ₊₁—C(R²¹)₂—. Preferably, W₁ is C2-C6 alkylene.

R²⁰ is —OH, —NH₂, —CH₃, C1-C3 alkylamine, C1-C3 dialkylamine, N-pyrrolidinyl, N-piperidinyl, N-morpholinyl, N-pyrazinyl, N′-acyl-N-pyrazinyl or N′-alkyl-N-pyrazinyl; preferably, R²⁰ is —OH, —OCH₃—NH₂, —NHCH₃, —N(CH₃)₂ or —CH₃.

Each R²¹ is —CH₃.

p is an integer from 1 to 4.

The remainder of the variables are as described above for Structural Formula (V) and (VI). 10 A second preferred set of values for the variables in Structural Formulas (V) and (VI) are provided in the following five paragraphs.

R¹ is —CONR¹¹R¹², —COOR¹², an optionally substituted heteroaryl group or an optionally substituted non-aromatic heterocyclic group.

W₁ is —C(R²¹)₂—W₄—.

W₄ is a C1-C5 alkylidene group optionally substituted with —OH, —NH₂, C1-C3 alkylamine, C1-C3 dialkylamine, N-pyrrolidinyl, N-piperidinyl, N-morpholinyl, N-pyrazinyl, N′-acyl-N-pyrazinyl or N′-alkyl-N-pyrazinyl or with one or more methyl groups. Preferably, the alkylidene group represented by W₄ is optionally substituted with —OH, —OCH₃—NH₂, —NHCH₃, —N(CH₃)₂ or one or more methyl groups.

Each R²¹ is independently —H or —CH₃. Preferably, each R²¹ is —H.

The remainder of the variables are as described above for Structural Formula (V) and (VI).

In a third preferred set of values for the variables in Structural Formulas (V) and (VI), R¹ is 2-piperidinyl, 3-piperidinyl, or 4-piperidinyl, and W₁ is a C1-C3 alkylidene. The remainder of the variables are as described above for Structural Formula (V) and (VI).

In a fourth preferred set of values for the variables in Structural Formulas (V) and (VI), R¹ is —NR¹¹R¹² and W₁ is a C2-C5 alkylene. More preferably, R¹ is —NHR¹², R¹² is —H or alkyl, and W₁ is a C2-C3 alkylene.

In one embodiment, the Chk-1 inhibitor is represented by Structural Formula (VII) or (VIIa):

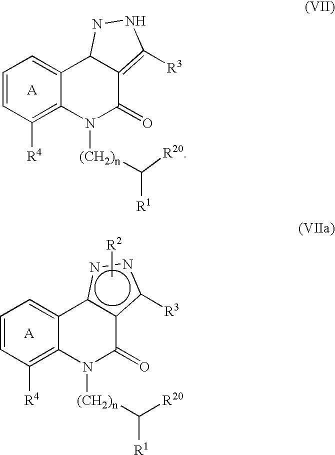

R¹ is an optionally substituted nitrogen-containing heteroaryl group, an optionally substituted non-aromatic nitrogen-containing heterocyclic group, —COOR¹² or —CONR¹¹R¹².

R¹¹ is —H and R¹² is cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, or —(CH₂)_{w}-pyrazinyl. Alternatively, —NR¹¹R¹² is N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl. The —(CH₂)_{w}-phenyl or —(CH₂)_{w}-pyridyl group represented by R¹² is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;

R²⁰ is —OH, —NH₂, —CH₃, C1-C3 alkylamine, C1-C3 dialkylamine, N-pyrrolidinyl, N-piperidinyl, N-morpholinyl, N-pyrazinyl, N′-acyl-N-pyrazinyl or N′-alkyl-N-pyrazinyl. Preferably, R²⁰ is —OH, —OCH₃—NH₂, —NHCH₃, —N(CH₃)₂ or —CH₃.

w is 0, 1 or 2.

n is an integer from 1 to 5.

The remainder of the variables as defined above for Structural Formula (V) and (VI).

In another preferred embodiment, the Chk-1 inhibitor is represented by Structural Formulas (VI) or (IX):

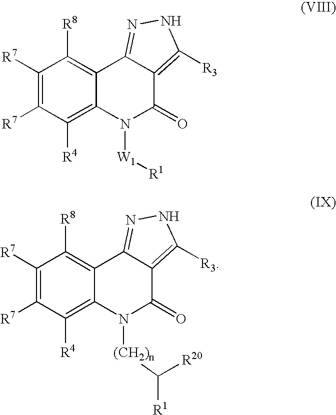

The variables for Structural Formulas (VIII) and (IX) are described in the following eight paragraphs.

R³ is —H, methyl, ethyl, n-propyl, iso-propyl, C1-C3 haloalkyl, or V₁—R^{3a}. Additional values for R³ include C3-C6 cycloalkyl and tetrahydrofuryl. V1 is a covalent bond or a C1-C2 alkylidene optionally substituted with one or two methyl groups or with a spiro cyclopropyl group; and R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidinyl, N-piperidinyl or N-morpholinyl. Preferably, R³ is methyl, ethyl, cyclopropyl, cyclopentyl, tetrahydrofuryl, or R³ is V₁—R^{3a}, wherein V₁ is a C1-C2 alkylidene and R^{3a} is OH or OCH₃.

Each R⁷ is independently —H, halogen, alkyl, haloalkyl, -T₁-V₃—R¹³, —NO₂, alkoxy, haloalkoxy or —CN. Additional values for R⁷ include —C≡CR²⁰¹, —C≡C—CH₂R²⁰², —C≡C—CH₂—CH₂R²¹², —CH═CHR²⁰¹, —CH═CH—CH₂R²⁰² and —CH═CH—CH₂—CH₂R²⁰².

R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃.

T₁ is a covalent bond, —O—, —NH—, —C(O)O—, —C(O)— or —C(O)NH—.

V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —NHC(O)NR¹⁴R¹⁵, —OC(O)NR¹⁴R¹⁵—NHC(O)OR¹⁴, —NHC(O)OR¹⁴, or a substituted or unsubstituted nitrogen-containing non-aromatic heterocyclic group (preferably attached to V₃ at a ring nitrogen atom). The C1-C4 alkylidene group represented by V₃ is optionally substituted with a spirocyclopropyl group or one or two methyl groups. Additionally, the C1-C4 alkylidene group represented by V₃ is optionally fused to a cyclopropyl group.

R¹³ is —CN, —OH, —NR¹⁴R¹⁵, —C(O)NR¹⁴R¹⁵, —NHC(O)R¹⁴, —NHC(O)NR¹⁴R⁵, —NHC(O)OR¹⁴ or an optionally substituted aromatic group or non-aromatic heterocyclic group. Additional values of R¹³ include —OR¹⁴ and —C(O)OR¹⁴.

Each R¹⁴ and each R¹⁵ is independently —H or C1-C3 alkyl or —NR¹⁴R¹⁵ is an optionally substituted non-aromatic heterocyclic group.

R²⁰¹ is —H, alkyl, haloalkyl, hydroxyalkyl, —CO₂R¹⁴ or an optionally substituted aromatic group or non-aromatic heterocyclic group;

R²⁰² is —H, —CN, —OR¹⁴, —OC(O)NR¹⁴R¹⁵, —OC(O)R¹⁴, —NR¹⁴R¹⁵, —C(O)NR¹⁴R¹⁵, —NR¹⁴C(O)R¹⁴, —NR¹⁴C(O)NR¹⁴R¹⁵, —NR¹⁴C(O)OR¹⁴, —NR¹⁴S(O)₂R^{x}, —S(O)₂NR¹⁴, —CO₂R¹⁴ or an optionally substituted aromatic group or non-aromatic heterocyclic group; and

R^{x} is alkyl or an optionally substituted aromatic group or non-aromatic heterocyclic group.

The remainder of the variables in Structural Formula (VIII) are as described for Structural Formulas (V) and (VI); and the remainder of the variables in Structural Formula (IX) are as described for Structural Formulas (VII) and (VIIa).

In Structural Formulas (VIII) and (IX), it is preferred that R¹, R³, R⁴, R⁷⁻⁸, R¹¹, R¹², R^{12a}, R^{12c}, R¹³, R²⁰², and V₃ are as defined below. The remainder of the variables are as described above.

R¹ in Structural Formula (VIII) is —OH, —CN, —OR¹², —NH₂, —NR¹¹R¹², N-pyrrolidinyl, N-piperidinyl, N-morpholinyl, N-pyrazinyl, N′-acyl-N-pyrazinyl, N′-alkyl-N-pyrazinyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, N-tetrahydroquinolinyl or N-tetrahydroisoquinolinyl. Alternatively, a second preferred set of values for R¹ in Structural Formula (VIII) is —NHCONR¹¹R¹², —OC(O)R¹²; NHC(O)OR¹², —O—C(O)—OR¹² or —O—C(O)—NR¹¹R¹². A third preferred set of values for R¹ is —NHCOR¹². A fourth preferred set of values for R₁ in Structural Formula (VIII) is —NR¹¹CO—CH(OR^{12a})R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —NHCO—CH(OH)—R¹², —NHCO—CH(NH₂)—R², —CH(OH)—CONR¹¹R¹², —CH(NH₂)—CONR¹², —OC(O)—CH(OH)—R¹² or —OC(O)—CH(NH₂)—R¹². When R¹ is selected from this fourth preferred set of values, W₁ is preferably C2-C5 alkylene. A fifth preferred set of values for R¹ is —NH₂, —NHCH₃, —N(CH₃)₂, N-pyrazinyl, N′-methyl-N-pyrazinyl, N-morpholinyl, 2-piperidinyl or 3-piperidinyl. When R¹ is selected from this fifth preferred set of values, W₁ is preferably C2-C5 alkylene or —(CH₂)ₚ—CH(CH₃)—CH₂—. A sixth set of preferred values for R¹ is —COOR¹² or —CONR¹¹R¹². When R₁ is selected from this preferred set of values, W₁ is preferably —CH₂—W₄— and W₄ is as defined above; and W₁ is more preferably C2-C5 alkylene. A seventh preferred set of values for R¹ is 2-piperidinyl, 3-piperidinyl, or 4-piperidinyl. When R₁ is selected from this seventh set of preferred values. W₁ is preferably a C1-C3 alkylidene. An eighth preferred set of values for R¹ is —NR¹¹R¹².

R₁ in Structural Formula (IX) is —CONR¹¹R¹².

R³ is —H, methyl, ethyl, n-propyl, iso-propyl, C1-C3 haloalkyl, or V₁—R^{3a}. Additional values for R³ include C3-C6 cycloalkyl and tetrahydrofuryl. V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl.

R⁴ and R⁸ are independently —H, halogen, —CH₃, halomethyl, —OCH₃, haloalkoxy.

One R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³. Additional values for R⁷ include —C≡CR²⁰¹ or —C≡C—CH₂R²¹². When R₁ is 2-piperidinyl, 3-piperidinyl, or 4-piperidinyl and W₁ is a C1-C3 alkylidene, then preferably each R⁷ is independently —H, —Cl, —F, —Br, alkyl, —OH, alkoxy, haloalkyl, haloalkoxy, —C(O)NH₂, —C(O)NHCH₃, —C(O)N(CH₃)₂, —NH₂, —NHCH₃, —N(CH₃)₂, —NHC(O)H, —NHC(O)CH₃, —V₃—R¹³ or —O—V₃—R¹³, with —C≡CR²⁰¹ or —C≡C—CH₂R²⁰² as additional values.

In Structural Formula (IX), R¹¹ and R¹² are as described in Structural Formula (VII). In Structural Formula (VIII), R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl or —(CH₂)_{w}-(optionally substituted aryl). Alternatively, —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl. Examples of values for —(CH₂)_{w}-(optionally substituted aryl) include —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Preferably, R¹² is alkyl or —(CH₂)_{w}-(optionally substituted aryl); and more preferably, R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen or —NO₂.

Each R^{12a} is defined above; preferably each R^{12a} is independently —H or —CH₃ or —NR^{12a}R^{12a} taken together is an aziridinyl group.

Each R^{12c} is defined above; preferably each R^{12c} is independently —H or —CH₃ or —C(R^{12c}R^{12c})— taken together is a cyclopropyl group.

R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N- piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Additional values for R¹³ include —C(O)OH, —C(O)OCH₃, oxazolyl, thiazolyl, thienyl, furyl, pyrimidinyl, pyrazinyl, N-alkyl-imidazolyl, pyrazolyl, and N-alkyl-pyrazolyl.

V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N- piperazinyl, N-pyrrolidyl, N-piperidinyl, or N-morpholinyl.

R²⁰² is —H, —OCH₃, —OCH₂CH₃, N-pyrrolidinyl, N-piperidinyl, N′-substituted-N-piperazinyl or N-morpholinyl.

w is 0, 1 or 2.

The remainder of the variables from this preferred set of values are defined as described above for Structural Formula (VIII) and (IX).

In another preferred embodiment, the disclosed Chk-1 inhibitors are represented by Structural Formula (XXXII):

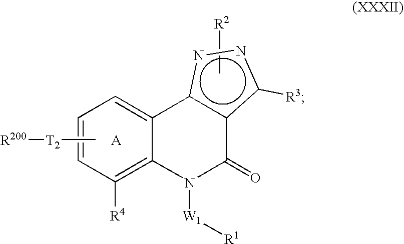

Ring A is optionally substituted at any one or more substitutable ring carbon atoms.

R²⁰⁰ is an optionally substituted aliphatic group.

T₂ is a covalent bond, —O—, —S—, —N(R⁶)—, —S(O)—, —SO₂—, —OC(O)—, —C(O)O—, —C(O)—, —N(R⁶)C(O)—, —C(O)N(R⁶)—, —SO₂N(R⁶)—, or —N(R⁶)SO₂—.

The remainder of the variables in Structural Formula (XXXII) are as described above for Structural Formula (I) or (V).

In another preferred embodiment, the Chk-1 inhibitor of the present invention is represented by Structural Formulas (XXXI) and (XXXIV):

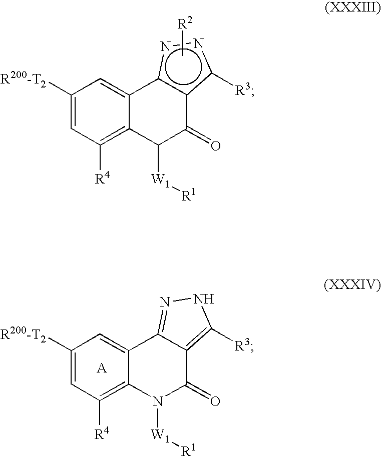

Ring A is optionally substituted at any one or more substitutable ring carbon atoms.

The remainder of the variables in Structural Formula (XXXII) and (XXXIV) are as described above for Structural Formula (XXXII).

One preferred set of values for the variables in Structural Formulas (XXXIII) (XXXIV) are described below in the following paragraphs.

R¹ and W₁ are as described in Structural Formula (XXXII). Preferably R₁ is —OR¹², —NR¹¹R¹², —CN, an optionally substituted nitrogen-containing heteroaryl group, an optionally substituted non-aromatic nitrogen-containing heterocyclic group, —NHCOR¹², —NHCONR¹¹R¹², —OC(O)R¹², NHC(O)OR¹², —O—C(O)—OR¹² or —O—C(O)—NR¹¹R¹²; W₁ is C2-C6 alkylene, —(CH₂)ₚ—CH(R²¹)—CH₂—, —(CH₂)ₚ—C(²¹)₂—CH₂— or —(CH₂)ₚ₊₁—C(R²¹)₂—; R²⁰ is —OH, —OCH₃—NH₂, —NHCH₃, —N(CH₃)₂ or —CH₃; each R²¹ is -CH₃; and p is an integer from 1 to 4. Alternatively, R¹ is —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R₁₂, —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —NHCO—CH(OH)—R¹², —NHCO—CH(NH₂)—R¹², —CH(OH)—CONR¹¹R¹², —CH(NH₂)—CONR¹², —OC(O)—CH(OH)—R¹², or —OC(O)—CH(NH₂)—R¹²; W₁ is C2-C6 alkylene, —(CH₂)ₚ—CH(R²⁰)—CH₂—, —(CH₂)ₚ—C(R²¹)₂—CH₂— or —(CH₂)ₚ₊₁—C(R²¹)₂—; R²⁰ is —OH, —OCH₃—NH₂, —NHCH₃, —N(CH₃)₂ or —CH₃; each R²¹ is —CH₃; and p is an integer from 1 to 4.

T₂ is a covalent bond.

Each R¹⁴ and each R¹⁵ is independently —H or C1-C3 alkyl or —NR¹⁴R¹⁵ is an optionally substituted non-aromatic heterocyclic group.

R²⁰⁰ is —C≡CR²⁰¹, —CH═CHR²⁰¹, —C≡C—CH₂R²⁰², —CH═CH—CH₂R²⁰², —C≡C—CH₂—CH₂R²⁰², —CH═CH—CH₂—CH₂R²⁰².

R²⁰¹ is —H, alkyl, haloalkyl, hydroxyalkyl, —CO₂R¹⁴, or an optionally substituted aromatic group or non-aromatic heterocyclic group.

R²⁰² is —H, —CN, —OR¹⁴, —OC(O)NR¹⁴R¹⁵, —OC(O)R¹⁴, —NR¹⁴R¹⁵, —C(O)NR¹⁴R¹⁵, —NR¹⁴C(O)R¹⁴, —NR¹⁴C(O)NR¹⁴R¹⁵, —NR¹⁴C(O)OR¹⁴, —NR¹⁴S(O)₂R^{x}, —S(O)₂NR¹⁴, —CO₂R¹⁴ or an optionally substituted aromatic group or non-aromatic heterocyclic group.

R^{x} is alkyl or an optionally substituted aromatic group or non-aromatic heterocyclic group.

The remainder of the variables for this preferred set are as described above for Structural Formulas (IV) or (XXXII).

In another preferred embodiment, the Chk-1 inhibitor of the present invention is represented by Structural Formula (XXXV):

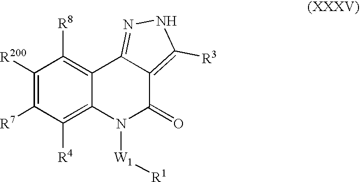

The variables for Structural Formula (XXXV) are described in the following paragraphs.

R³ is —H, methyl, ethyl, n-propyl, iso-propyl, C3-C6 cycloalkyl, tetrahydrofuryl, C1-C3 haloalkyl or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl.

R⁷ is —H, halogen, alkyl, haloalkyl, -T₁-V₃—R¹³, —NO₂, alkoxy, haloalkoxy or —CN.

R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃.

T₁ is a covalent bond, —O—, —NH—, —C(O)O—, —C(O)— or —C(O)NH—.

V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵—NHC(O)OR¹⁴, —NHC(O)OR¹⁴, or a substituted or unsubstituted nitrogen-containing non-aromatic heterocyclic group (preferably attached to V₃ at a ring nitrogen atom) wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group.

R¹³ is —CN, —OR¹⁴, —NR¹⁴R₁₅, —C(O)NR¹⁴R¹⁵, —NHC(O)R¹⁴, —NHC(O)NR¹⁴R¹⁵, —NHC(O)OR¹⁴, —C(O)OR¹⁴ or an optionally substituted aromatic group or non-aromatic heterocyclic group.

The remainder of the variables in Structural Formula (XXXV) are as described for Structural Formulas (XXIX) and (XXXIV).

In Structural Formula (XXXV), it is preferred that R¹, R³, R⁴, R⁷⁻⁸, R¹¹, R¹², R^{12a}, R^{12c}, R¹³, R²⁰⁰, R²⁰² and V₃ are as defined below. The remainder of the variables are as described above.

R¹ in Structural Formula (XXXV) is —OH, —CN, —OR¹², —NH₂, —NR₁₁R¹², N-pyrrolidinyl, N-piperidinyl, N-morpholinyl, N-pyrazinyl, N′-acyl-N-pyrazinyl, N′-alkyl-N-pyrazinyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, N-tetrahydroquinolinyl or N-tetrahydroisoquinolinyl. Alternatively, a second preferred set of values for R¹ in Structural Formula (VIII) is —NHCONR¹¹R¹², —OC(O)R¹²; NHC(O)OR¹², —O—C(O)—OR¹² or —O—C(O)—NR¹¹R¹². A third preferred set of values for R¹ is —NHCOR¹². A fourth preferred set of values for R¹ in Structural Formula (VIII) is —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹²—NHCO—CH(OH)—R¹², —NHCO—CH(NH₂)—R¹², —CH(OH)—CONR¹¹R¹², —CH(NH₂)—CONR¹², —OC(O)—CH(OH)—R¹² or —OC(O)—CH(NH₂)—R¹². When R¹ is selected from this fourth preferred set of values, W₁ is preferably C2-C6 alkylene, —(CH₂)ₚ—CH(R²⁰)—CH₂—, —(CH₂)ₚ—C(R²¹)₂—CH₂— or —(CH₂)ₚ₊₁—C(R²¹)₂—; R²⁰ is —OH, —OCH₃—NH₂, —NHCH₃, —N(CH₃)₂ or —CH₃; each R²¹ is —CH₃; and p is an integer from 1 to 4. More preferably, W₁ is C2-C5 alkylene. A fifth preferred set of values for R¹ is —NH₂, —NHCH₃, —N(CH₃)₂, N-pyrazinyl, N′-methyl-N-pyrazinyl, N-morpholinyl, 2-piperidinyl or 3-piperidinyl. When R₁ is selected from this fifth preferred set of values, W₁ is preferably C2-C5 alkylene or —(CH₂)ₚ—CH(CH₃)—CH₂—. A sixth set of preferred values for R₁ is —COOR¹² or —CONR¹¹R¹². When R₁ is selected from this preferred set of values, W₁ is preferably —CH₂—W₄— and W₄ is as defined above; and W₁ is more preferably C2-C5 alkylene. A seventh preferred set of values for R₁ is 2-piperidinyl, 3-piperidinyl, or 4-piperidinyl. When R¹ is selected from this seventh set of preferred values. W₁ is preferably a C1-C3 alkylidene. An eighth preferred set of values for R₁ is —NR¹¹R¹².

R³ is methyl, ethyl, cyclopropyl, cyclopentyl, tetrahydrofuryl, or R³ is V₁—R^{3a}, wherein V₁ is a C1-C2 alkylidene and R^{3a} is OH or OCH₃.

R⁴ and R⁸ are independently —H, halogen, —CH₃, halomethyl, —OCH₃, haloalkoxy.

R⁷ is —H, —Cl, —F, —Br, alkyl, —OH, alkoxy, haloalkyl, haloalkoxy, —C(O)NH₂, —C(O)NHCH₃, —C(O)N(CH₃)₂, —NH₂, —NHCH₃, —N(CH₃)₂, —NHC(O)H, —NHC(O)CH₃, —V₃—R¹³ or —O—V₃—R¹³.

R₁₁ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl or —(CH₂)_{w}-(optionally substituted aryl). Alternatively, —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl. Examples of values for —(CH₂)_{w}-(optionally substituted aryl) include —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Preferably, R¹² is alkyl or —(CH₂)_{w}-(optionally substituted aryl); and more preferably, R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen or —NO₂.

Each R^{12a} is defined above; preferably each R^{12a} is independently —H or —CH₃ or —NR^{12a}R^{12a} taken together is a aziridinyl group.

Each R^{12c} is defined above; preferably each R^{12c} is independently —H or —CH₃ or —C(R^{12c}R^{12c})— taken together is a cyclopropyl group.

R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N- piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Additional values for R¹³ include —C(O)OH, —C(O)OCH₃, oxazolyl, thiazolyl, thienyl, furyl, pyrimidinyl, pyrazinyl, N-alkyl-imidazolyl, pyrazolyl, and N-alkyl-pyrazolyl.

V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen, N-piperazinyl, N′-alkyl-N-piperazinyl, N′-acyl-N- piperazinyl, N-pyrrolidyl, N-piperidinyl, or N-morpholinyl.

Even more preferably, the Chk-1 inhibitor is represented by Structural Formula (XXXV), R²⁰⁰ and R²⁰¹ are defined in the following two paragraphs and the remainder of the variables are as defined above.

R²⁰⁰ is —C≡CR²⁰¹ or —C≡C—CH₂R²⁰².

R²⁰² is —H, —OCH₃, —OCH₂CH₃, N-pyrrolidinyl, N-piperidinyl, N′-substituted-N-piperazinyl or N-morpholinyl.

In another preferred embodiment, the Chk-1 inhibitor of the present invention is represented by Structural Formula (XXXVI):

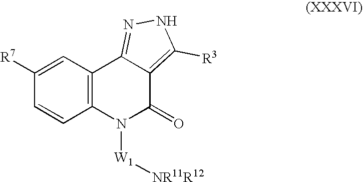

The variables for Structural Formula (XXXVI) are described in the following paragraphs.

W₁ is C2-C4 alkylidene optionally substituted with a methyl group or a gemdimethyl group, —(CH₂)—CH(R²⁰)—CH₂—, or —(CH₂)₂—CH(R²⁰)—CH₂—. W₁ is preferably a C2-C4 alkylene.

R³ is methyl, ethyl, cyclopropyl, cyclopentyl, or tetrahydrofuryl; or R³ is V₁—R^{3a}, wherein V₁ is a C1-C2 alkylidene and R^{3a} is —OH, —OCH₃.

R⁷ is halogen, alkyl, haloalkyl, —C≡CR²⁰¹, —CH═CHR²⁰¹, —C≡C—CH₂R²⁰², —CH═CH—CH₂R²⁰², —C≡C—CH₂—CH₂R²⁰², —CH═CH—CH₂—CH₂R²⁰², an optionally substituted heteroaryl, —NR¹⁴R¹⁵, —CH₂NR¹⁴R¹⁵, T₁-V₃—NR¹⁴R¹⁵. Preferred heteroaryl groups represented by R⁷ include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, benzisoxazolyl, tetrahydroquinolinyl, tetrahydroisoquinolinyl, and pyrido [3, 4-d]pyrimidinyl. More preferred heteroaryl groups for R⁷ include 4-pyridyl, 3-pyrazolyl, 4-pyrazolyl, N-methyl-3-pyrazolyl, N-methyl-4-pyrazolyl, 2-oxazolyl, 4-oxazolyl, 5-oxazolyl, 2-thiazolyl, 4-thiazolyl, 5-thiazolyl, 2-furanyl, 3-furanyl, 2-thienyl, 3-thienyl, 2-imidazolyl, 4-imidazolyl, 5-imidazolyl, N-methyl-2-imidazolyl, N-methyl-4-imidazolyl, N-methyl-5-imidazolyl, 2-pyrrolyl, 3-pyrrolyl, N-methyl-2-pyrrolyl, N-methyl-3-pyrrolyl, 3-isoxazolyl, 4-isoxazolyl, 5-isoxazolyl, 5-triazolyl, and tetrazolyl.

T₁ is a covalent bond, —O—, —NH—, —C(O)O—, —C(O)— or —C(O)NH—. T₁ is preferably a covalent bond.

V₃ is a covalent bond or a C2-C4 alkylidene optionally substituted with a spirocyclopropyl group or one or two methyl groups.

Each R¹¹ and each R¹² is independently —H or alkyl, or —NR¹¹R¹² is a non-aromatic heterocyclic group optionally N-substituted at any substitutable ring nitrogen atom. In one embodiment —NR¹¹R¹² is morpholinyl, thiomorpholinyl, pyrrolidinyl, piperazinyl, piperazinyl, piperidinyl, pyrrolidinyl, thiazolidinyl, diazolonyl, diazolonyl, 1-pthalimidinyl, benzopyrrolidinyl, benzopiperidinyl, indolinyl, phenanthridinyl, 3-1-H-benzimidazol-2-one, or tetrahydroquinolinyl, optionally substituted at a substitutable ring nitrogen with —R\hat{ }, —N(R\hat{ })₂, —C(O)R\hat{ }, —CO₂R\hat{ }, —C(O)C(O)R\hat{ }, —C(O)CH₂C(O)R\hat{ }, —SO₂R\hat{ }, —SO₂N(R\hat{ })₂, —C(═S)N(R\hat{ })₂, —C(═NH)—N(R\hat{ })₂, or —NR\hat{ }SO₂R\hat{ }; wherein R\hat{ }is hydrogen, an alkyl group, phenyl (Ph) or CH₂(Ph). Preferably substituents for a substituted ring nitrogen are —R\hat{ }, —COR\hat{ }, and COOR\hat{ }.

Each R¹⁴ and each R¹⁵ is independently —H or C1-C3 alkyl or —NR¹⁴R¹⁵ is a non-aromatic heterocyclic group optionally N-substituted at any substitutable ring nitrogen atom. In one embodiment —NR¹⁴R¹⁵ is morpholinyl, thiomorpholinyl, pyrrolidinyl, piperazinyl, piperazinyl, piperidinyl, pyrrolidinyl, thiazolidinyl, diazolonyl, diazolonyl, 1-pthalimidinyl, benzopyrrolidinyl, benzopiperidinyl, indolinyl, phenanthridinyl, 3-1-H-benzimidazol-2-one, or tetrahydroquinolinyl, optionally substituted at a substitutable ring nitrogen with —R\hat{ }, —N(R\hat{ })₂, —C(O)R\hat{ }, —CO₂R\hat{ }, —C(O)C(O)R\hat{ }, —C(O)CH₂C(O)R\hat{ }, —SO₂R\hat{ }, —SO₂N(R\hat{ })₂, —C(═S)N(R\hat{ })₂, —C(═NH)—N(R\hat{ })₂, or —NR\hat{ }SO₂ R\hat{ }; wherein R\hat{ } is hydrogen, an alkyl group, phenyl (Ph) or CH₂(Ph). Preferably substituents for a substituted ring nitrogen are —R\hat{ }, —COR\hat{ }, and COOR\hat{ }.

R²⁰ is —OH, —OCH₃—NH₂, —NHCH₃, —N(CH₃)₂ or —CH₃.

R²⁰¹ is —H, alkyl, haloalkyl, hydroxyalkyl, —CO₂R¹⁴, or an optionally substituted aromatic group or non-aromatic heterocyclic group.

R²⁰² is —H, —CN, —OR¹⁴, —OC(O)NR¹⁴R¹⁵, —OC(O)R¹⁴, —NR¹⁴R¹⁵, —C(O)NR¹⁴R¹⁵, —NR¹⁴C(O)R¹⁴, —NR¹⁴C(O)NR¹⁴R¹⁵, —NR¹⁴C(O)OR¹⁴, —NR¹⁴S(O)₂R^{x}, —S(O)₂NR¹⁴, —CO₂R¹⁴ or an optionally substituted aromatic group or non-aromatic heterocyclic group.

R^{x} is alkyl or an optionally substituted aromatic group or non-aromatic heterocyclic group.

In another preferred embodiment, the Chk-1 inhibitor is represented by Structural Formula (IXa):

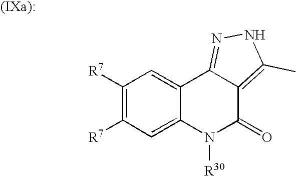

Each R⁷ is as defined for Structural Formula (VII) above.

R³⁰ is a structural formula selected from:

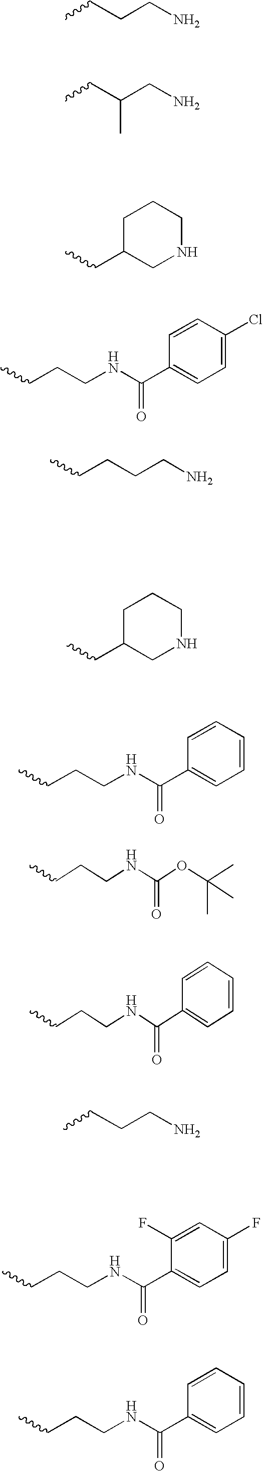

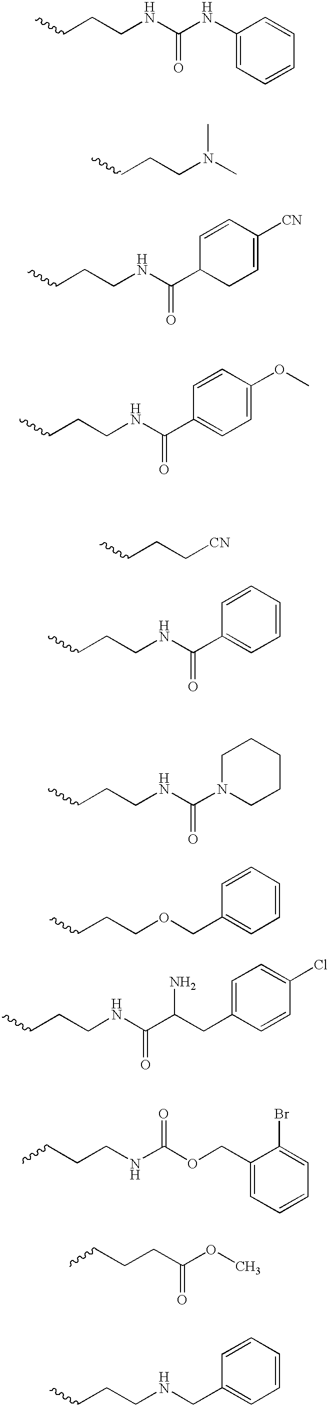

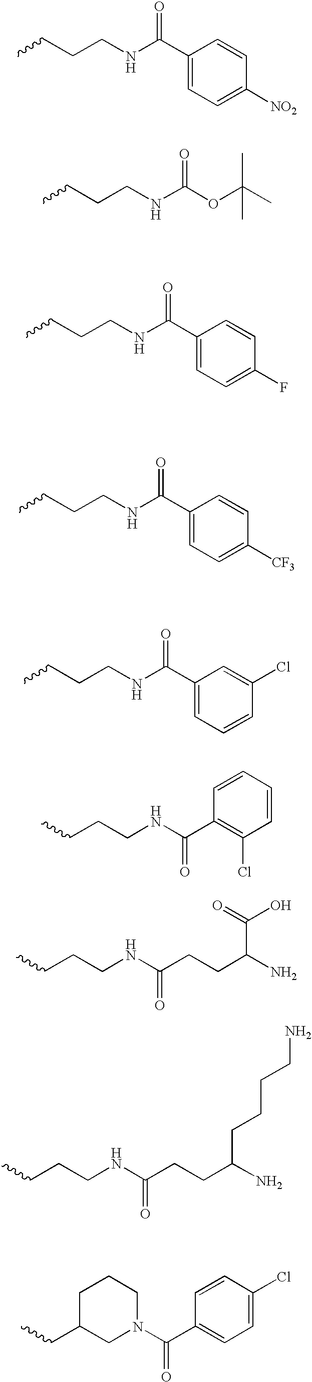

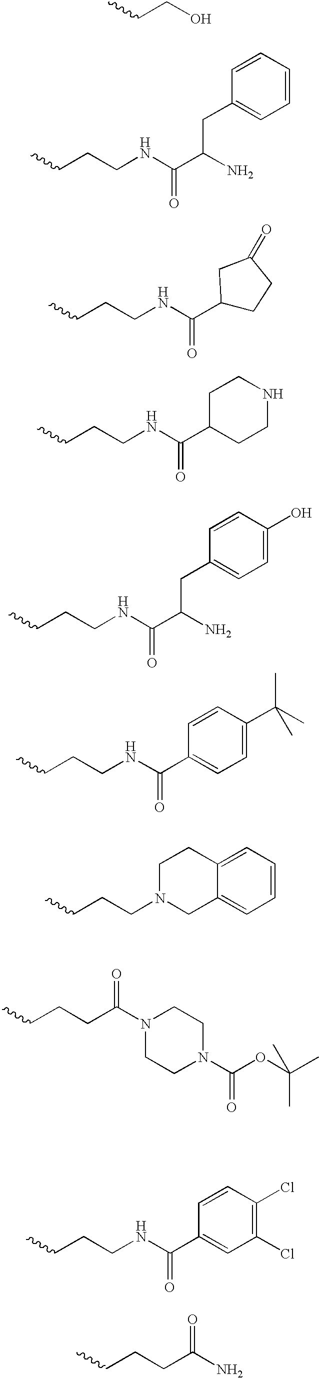

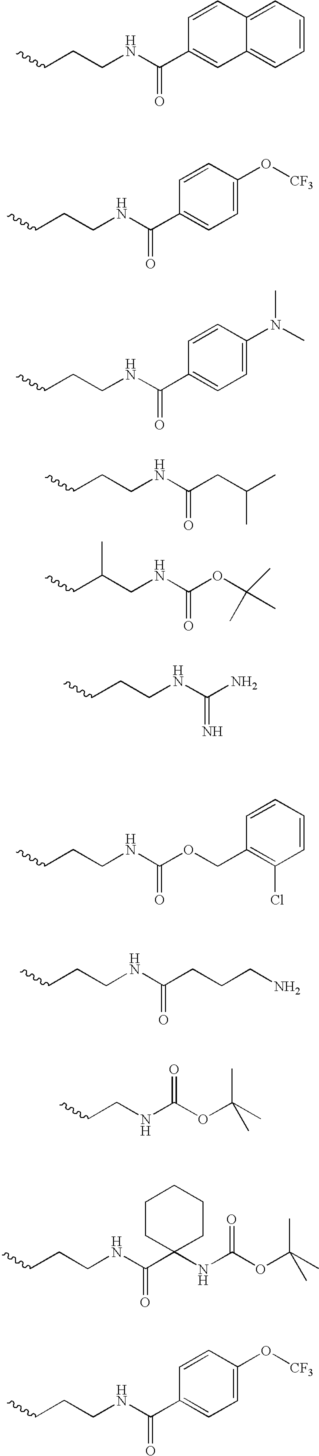

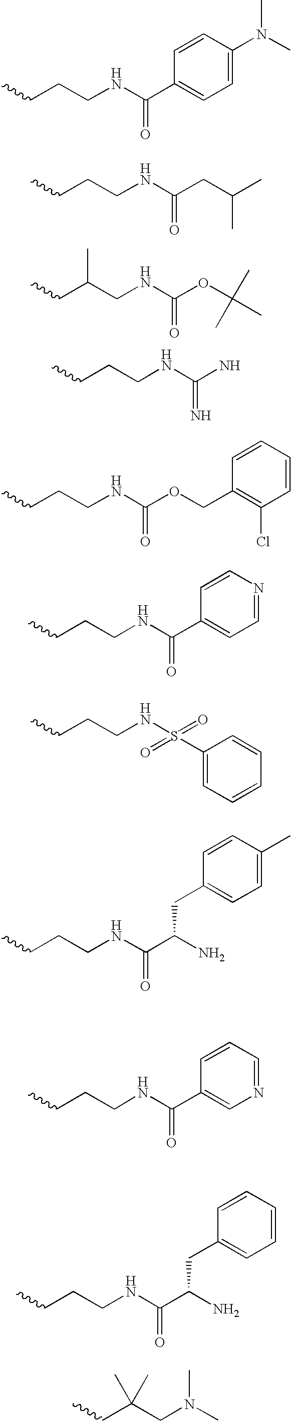

The “jagged” line in the structural formulas shown directly above indicates the bond by which the group is connected to the remainder of the molecule, i.e., the bond by which the quinolinone nitrogen atom in Structural Formula (IXa) is connected to the indicated group.

Another preferred embodiment or the present invention, the Chk-1 inhibitor is represented by Structural Formulas (I)-(IX), provided that —W₁—R¹ is R³⁰, as defined in the previous paragraph.

In another preferred embodiment, the disclosed Chk-1 inhibitor is represented by Structural Formulas (X) or (XI):

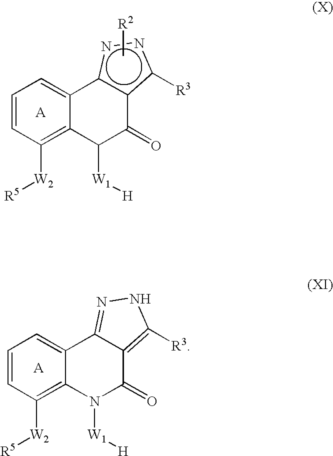

Ring A in Structural Formulas (X) or (XI) is optionally substituted at any one or more substitutable ring carbon atoms.

The remainder of the variables in Structural Formulas (X) and (XI) are as described above for Structural Formula (I).

In another preferred embodiment, the Chk-1 inhibitor of the present invention is represented by Structural Formulas (XII) or (XI):

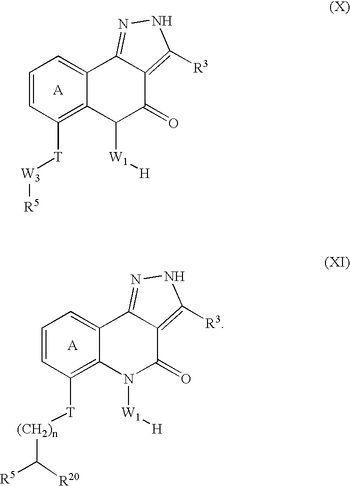

In Structural Formulas (XII), R⁵ is —OR¹², —NR¹¹R¹², —CN, an optionally substituted nitrogen-containing heteroaryl group, an optionally substituted non-aromatic nitrogen-containing heterocyclic group, —NHCOR¹², —OC(O)R¹², —NHC(O)NR¹⁴R¹⁵, —OC(O)NR¹⁴R¹⁵—NHC(O)OR¹⁴ or —NHC(O)OR¹⁴. Alternatively in Structural Formula (XII), R⁵ is —NR¹¹CO—CH(OR^{12a})—R¹², —NR¹¹CO—CH(NR^{12a}R^{12a})—R¹², —OC(O)—CH(OR^{12a})—R₁₂, —OC(O)—CH(NR^{12a}R^{12a})—R¹², —NR¹¹CO—C(R^{12c}R^{12c})—OR¹², —NR¹¹CO—C(R^{12c}R^{12c})—NR₁₁R¹², —OC(O)—C(R^{12c}R^{12c})—OR¹², —OC(O)—C(R^{12c}R^{12c})—NR¹¹R¹², —NHCO—CH(OH)—R¹², —NHCO—CH(NH₂)—R¹², —CH(OH)—CONR¹¹R¹², —CH(NH₂)—CONR¹², —OC(O)—CH(OH)—R¹², —OC(O)—CH(NH₂)—R¹². In a second alternative, R¹ in Structural Formula (XII) is an optionally substituted nitrogen-containing heteroaryl group, an optionally substituted non-aromatic nitrogen-containing heterocyclic group, COOR¹² or —CONR¹¹R¹². In Structural Formula (XIII), R⁵ is an optionally substituted nitrogen-containing heteroaryl group, an optionally substituted non-aromatic nitrogen-containing heterocyclic group, COOR¹² or —CONR¹¹R¹².

T is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—. Preferably, T is a covalent bond or —O—.

W₃ is a linear C2-C5 alkylidene chain, optionally monosubstituted with —OR^{12b}, —N(R^{12b})₂, or a spiro cycloalkyl group and/or is optionally substituted with one or more —CH₃ groups and wherein W₃ optionally has a cyclopropyl, monomethyl cyclopropyl or dimethyl cyclopropyl group fused thereto. Preferably, W₃ is C2-C5 alkylene, —(CH₂)ₚ—CH(R²⁰)—CH₂—, —(CH₂)ₚ—C(R²¹)₂—CH₂—, —(CH₂)ₚ₊₁—C(R²¹)₂— or —(CH₂)ᵣ—CH(R²²)CH(R²²)—CH₂—.

R²⁰ is —OH, —OCH₃—NH₂, —NHCH₃, —N(CH₃)₂ or —CH₃.

Each R²¹ is —CH₃.

Both R²²s, taken together, are >CH₂, >CHCH₃ or >C(CH₃)₂.

p is an integer from 1 to 3 and r is 1 or 2.

n is an integer from 2 to 5.

The remainder of the variables in Structural Formulas (XII) and (XII) are as described above for Structural Formulas (X) and (XI).

In another preferred embodiment, the Chk-1 inhibitor of the present invention is represented by Structural Formulas (XIV) or (XV):

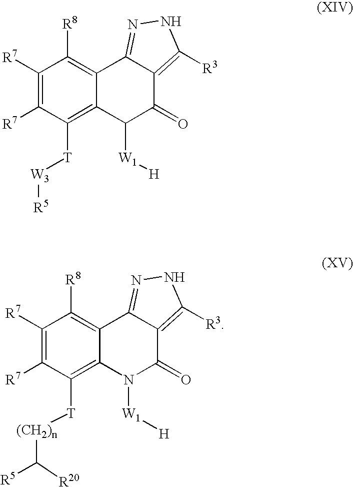

Definitions for the variables in Structural Formulas (XIV) and (XV) are provided in the following eight paragraphs.

R³ is —H, methyl, ethyl, n-propyl, iso-propyl, C1-C3 haloalkyl or V₁—R^{3a}. Additional values for R³ include C3-C6 cycloalkyl and tetrahydrofuryl. V₁ is a covalent bond or a C1-C2 alkylidene optionally substituted with one or two methyl groups or with a spiro cyclopropyl group; and R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N- piperazinyl, N-pyrrolidyl, N-piperidinyl or N-morpholinyl. Preferably, R³ is methyl, ethyl, cyclopropyl, cyclopentyl, tetrahydrofuryl, or R³ is V₁—R^{3a}, wherein V₁ is a C1-C2 alkylidene and R^{3a} is OH or OCH₃.

Each R⁷ is independently —H, halogen, alkyl, haloalkyl, -T₁-V₃—R¹³, —NO₂, alkoxy, haloalkoxy or —CN.

R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃.

T₁ is a covalent bond, —O—, —NH—, —C(O)O—, —C(O)— or —C(O)NH—.

V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —NHC(O)NR¹⁴R¹⁵, —OC(O)NR¹⁴R¹⁵—NHC(O)OR¹⁴, —NHC(O)OR¹⁴, or a substituted or unsubstituted nitrogen-containing non-aromatic heterocyclic group (preferably attached to V₃ at a ring nitrogen atom). The C1-C4 alkylidene group represented by V₃ is optionally substituted with a spirocyclopropyl group or one or two methyl groups. Additionally, the C1-C4 alkylidene group represented by V₃ is optionally fused to a cyclopropyl group.

R¹³ is —CN, —OH, —NR¹⁴R¹⁵, —C(O)NR¹⁴R¹⁵, —NHC(O)R¹⁴, —NHC(O)NR¹⁴R¹⁵, —NHC(O)OR¹⁴ or an optionally substituted aromatic group or non-aromatic heterocyclic group. Additional values for R¹³ include —OR¹⁴ and —C(O)OR¹⁴.

R¹⁴ and R¹⁵ are independently —H or C1-C3 alkyl or —NR¹⁴R¹⁵ is an optionally substituted non-aromatic heterocyclic group.

The remainder of the variables in Structural Formula (XIV) are as described in Structural Formula (XII); and the remainder of the variables in Structural Formula (XV) are as described in Structural Formula (XIII).

In Structural Formulas (XIV) and (XV), it is preferred that R³, R⁵, R⁷⁻⁸, R¹¹, R¹², R^{12a}, R^{12c}, R¹³, and V₃ are as defined below.

R³ is —H, methyl, ethyl, n-propyl, iso-propyl, C1-C3 haloalkyl, or V₁—R^{3a}. Additional values for R³ include C3-C6 cycloalkyl and tetrahydrofuryl. V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl.

R⁵ is —OH, —CN, —OR¹², —NH₂, —NR¹¹R¹², N-pyrrolidinyl, N-piperidinyl, N-morpholinyl, N-pyrazinyl, N′-acyl-N-pyrazinyl, N′-alkyl-N-pyrazinyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, N-tetrahydroquinolinyl or N-tetrahydroisoquinolinyl. Alternatively, a second preferred set of values for R⁵ in Structural Formula (XIV) is —NHCONR¹¹R¹², —OC(O)R¹²; NHC(O)OR¹², —O—C(O)—OR¹² or —O—C(O)—NR¹¹R¹². A third preferred set of values for R⁵ in Structural Formulas (XIV) is —NHCOR¹². A fourth preferred set of values for R⁵ in Structural Formula (XIV) is —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —NHCO—CH(OH)—R¹², —NHCO—CH(NH₂)—R¹², —CH(OH)—CONR¹¹R¹², —CH(NH₂)—CONR¹², —OC(O)—CH(OH)—R¹², —OC(O)—CH(NH₂)—R¹². When R⁵ is selected from this fourth preferred set of values, W₃ is preferably C2-C5 alkylene. A fifth preferred set of values for R⁵ in Structural Formula (XIV) is —NH₂, —NHCH₃, —N(CH₃)₂, N-pyrazinyl, N′-methyl-N-pyrazinyl, N-morpholinyl, 2-piperidinyl or 3-piperidinyl. When R⁵ is selected from this fifth preferred set of values, W₃ is preferably C2-C5 alkylene or —(CH₂)ₚ—CH(CH₃)—CH₂—. A sixth set of preferred values for R⁵ is —COOR¹² or —CONR¹¹R¹².

R⁵ in Structural Formula (XV) is —CONR¹¹R¹².

One R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R³or —O—V₃—R¹³.

R⁸ is —H, halogen, —CH₃, halomethyl, —OCH₃, haloalkoxy.

In Structural Formula (XV), R¹¹ and R¹² are as described in Structural Formula (XIII). In Structural Formula (XIV), R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl or —(CH₂)_{w}-(optionally substituted aryl). Alternatively, —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl. Examples of values for —(CH₂)_{w}-(optionally substituted aryl) include —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen. Preferably, R¹² is alkyl or —(CH₂)_{w}-(optionally substituted aryl). More preferably, R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen or —NO₂.

Each R^{12a} is defined above; preferably each R^{12a} is independently —H or —CH₃ or —NR^{12a}R^{12a} taken together is an aziridinyl group.

Each R^{12c} is defined above; preferably each R^{12c} is independently —H or —CH₃ or —C(R^{12c}R^{12c})— taken together is a cyclopropyl group.

R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen.

V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl.

w is 0, 1 or 2.

The remainder of the variables from this preferred set of values are defined as described above for Structural Formula (XII) and (XIII).

Specific examples of Chk-1 inhibitors of the present invention are provided below as Compounds 1-436

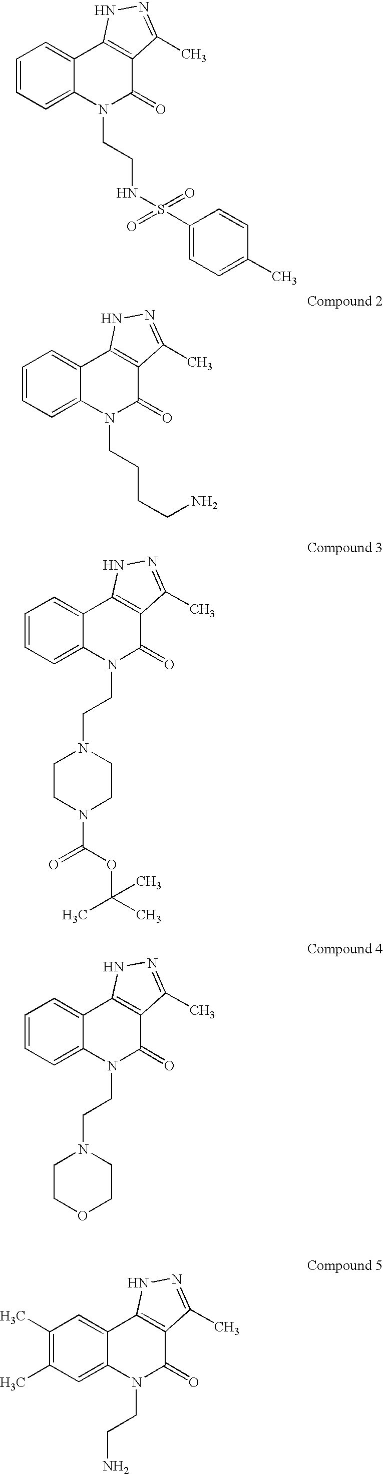

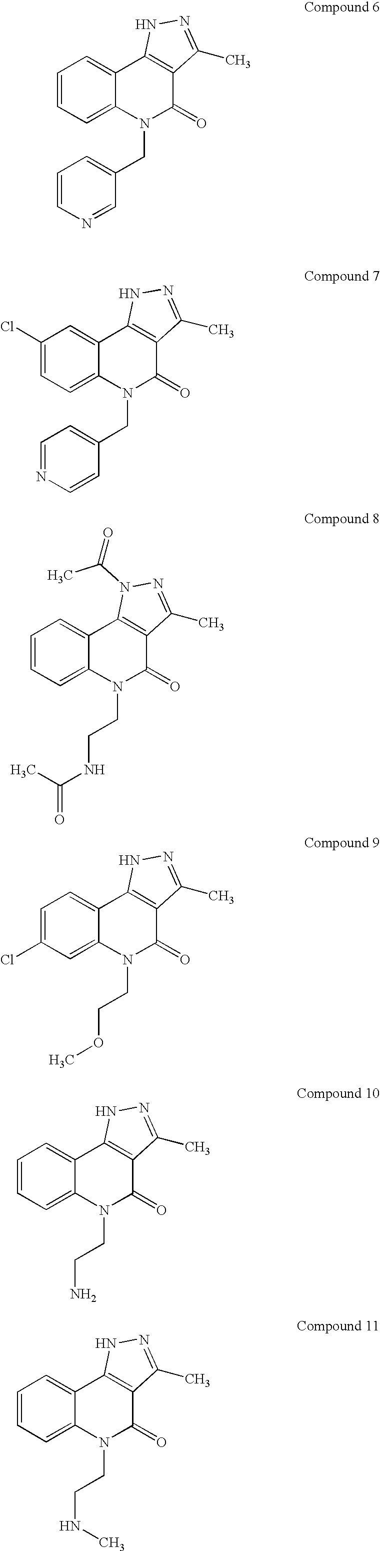

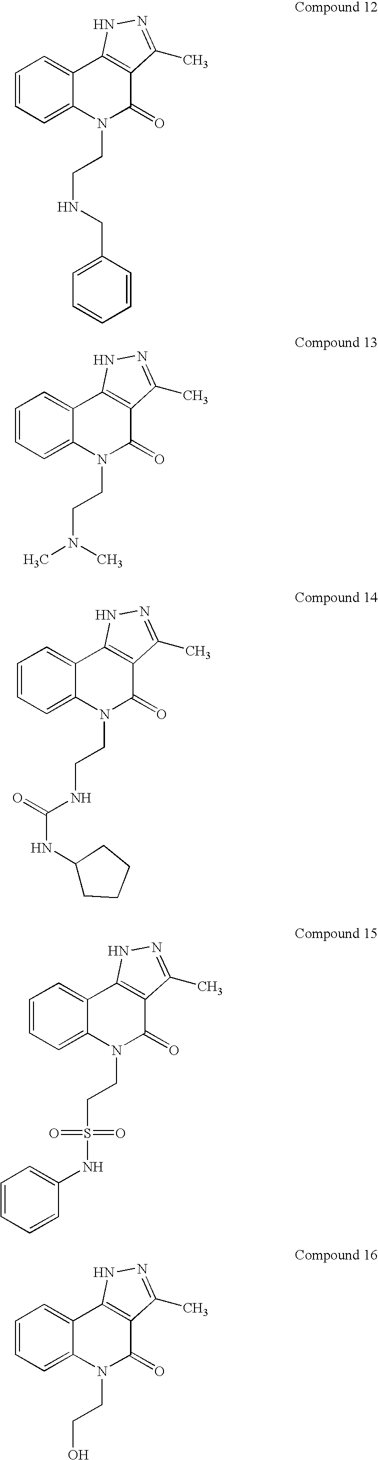

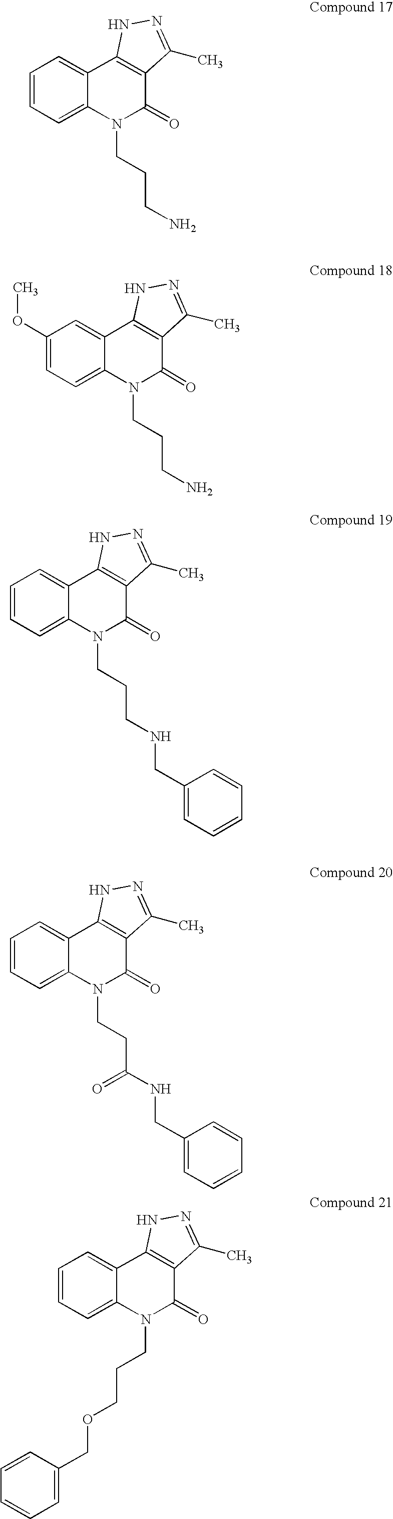

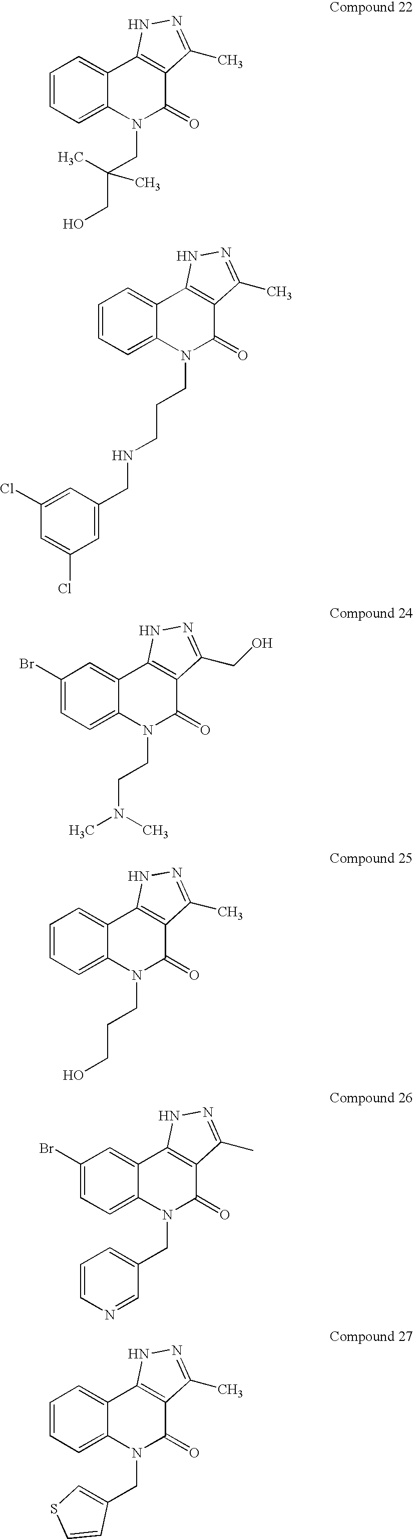

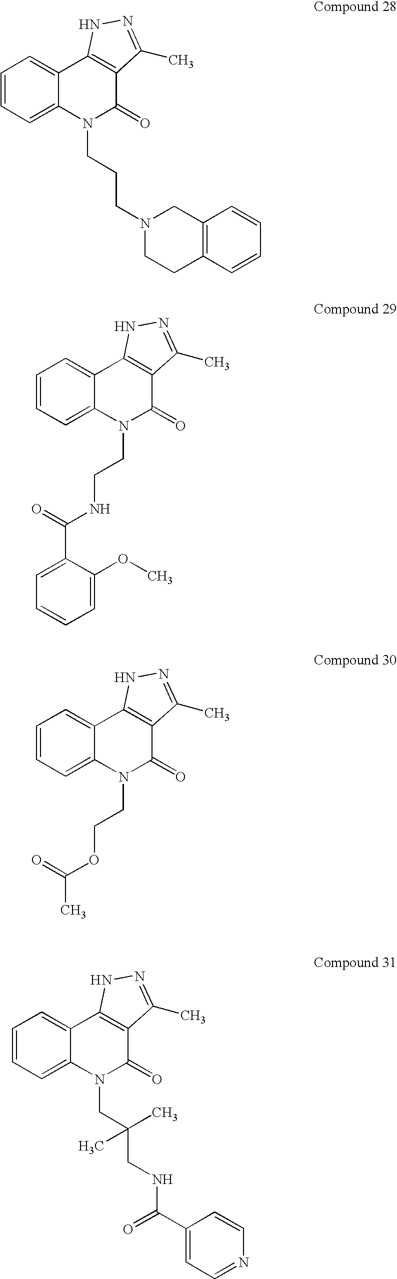

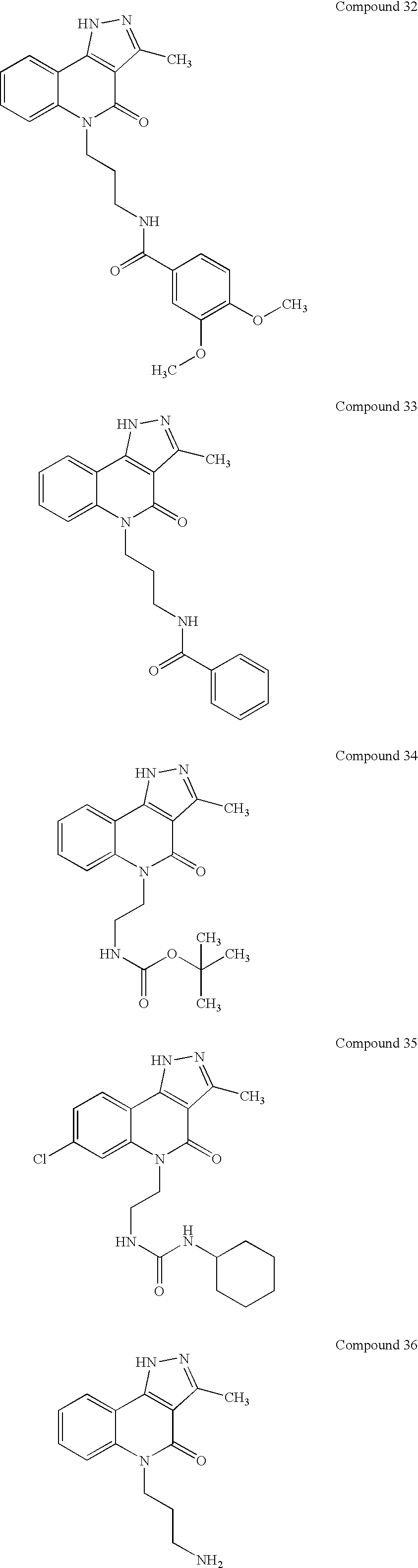

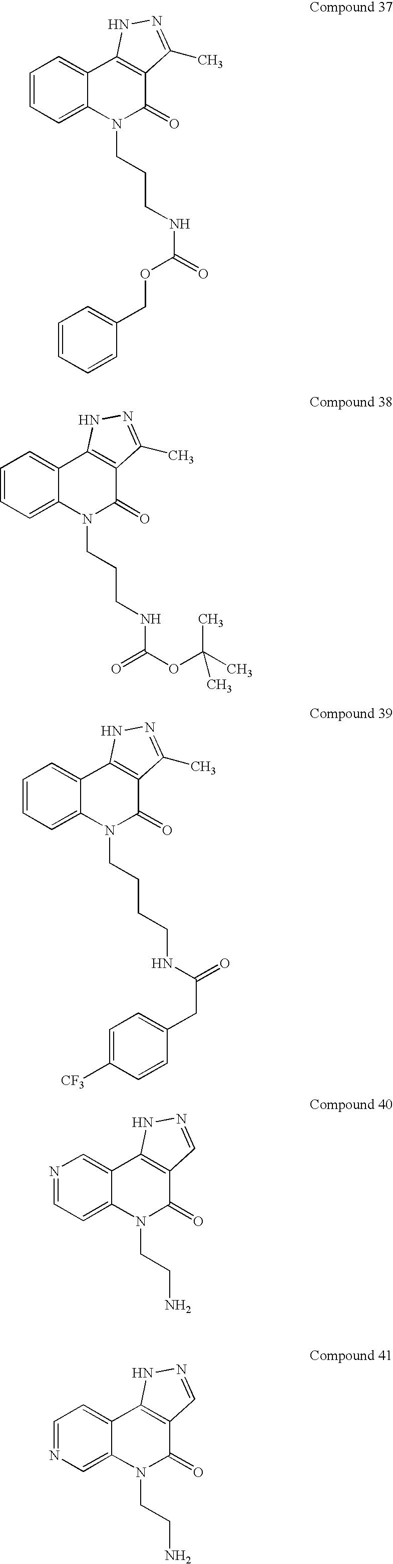

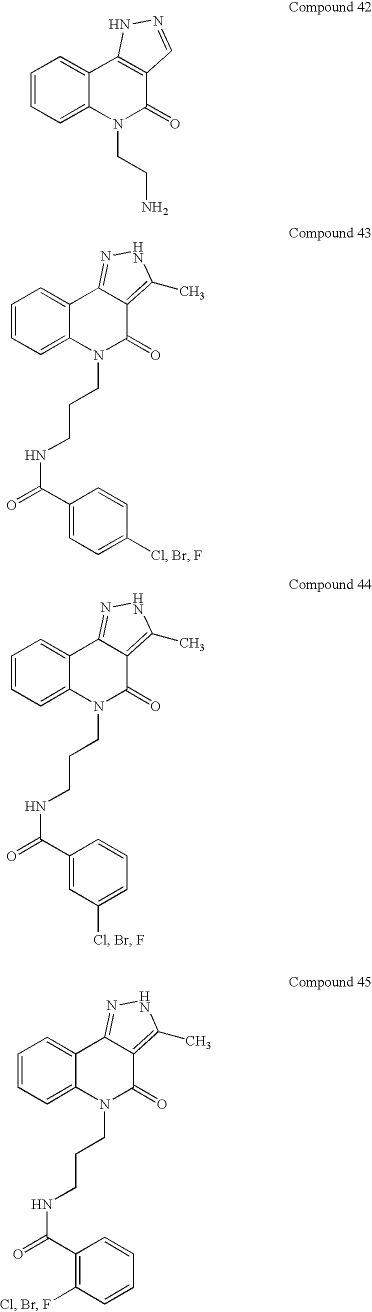

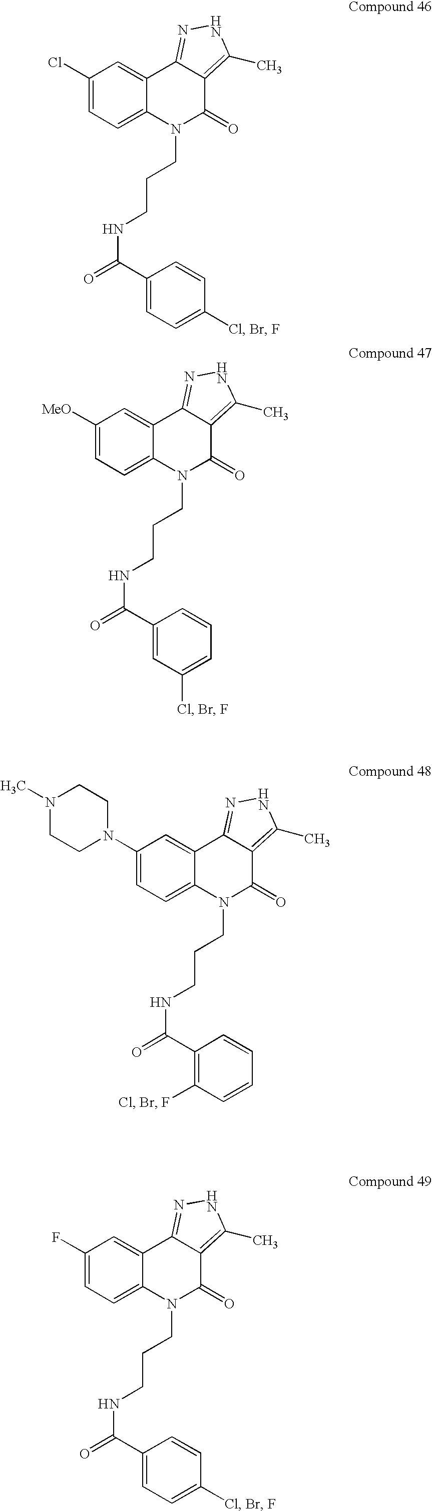

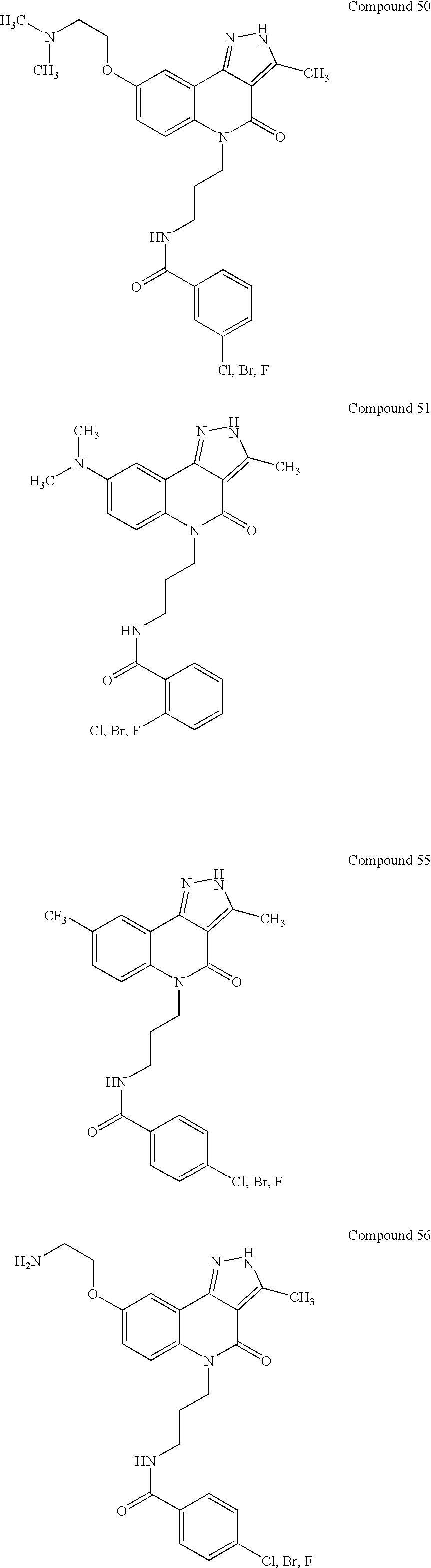

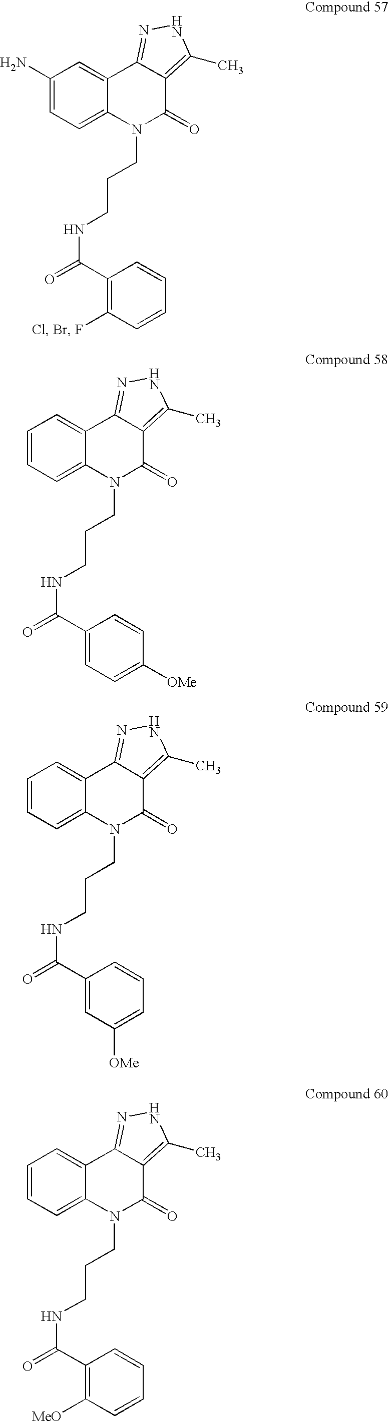

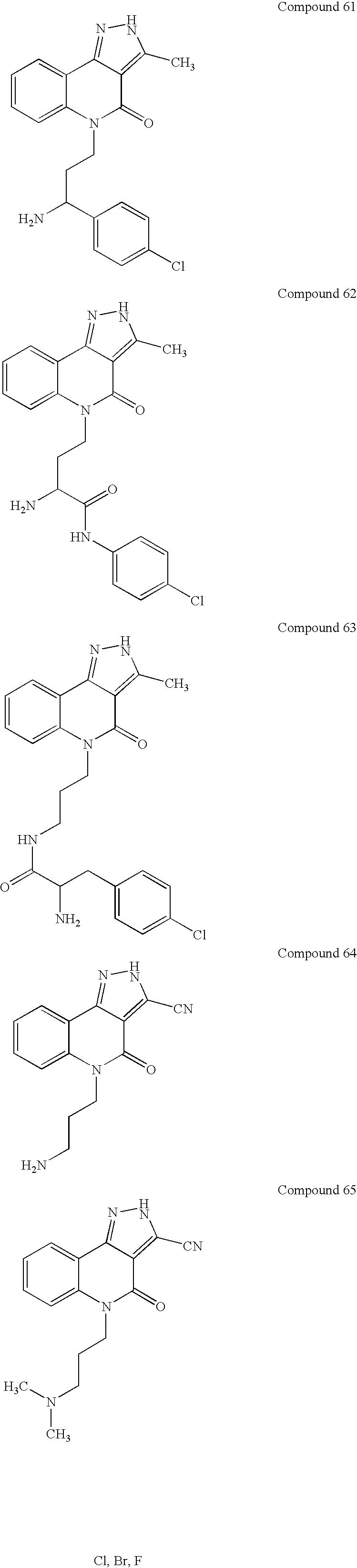

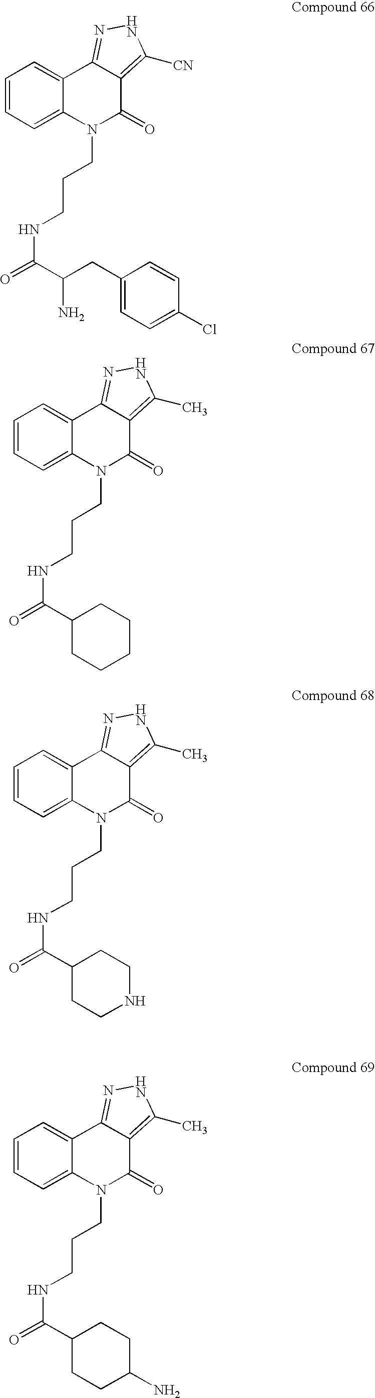

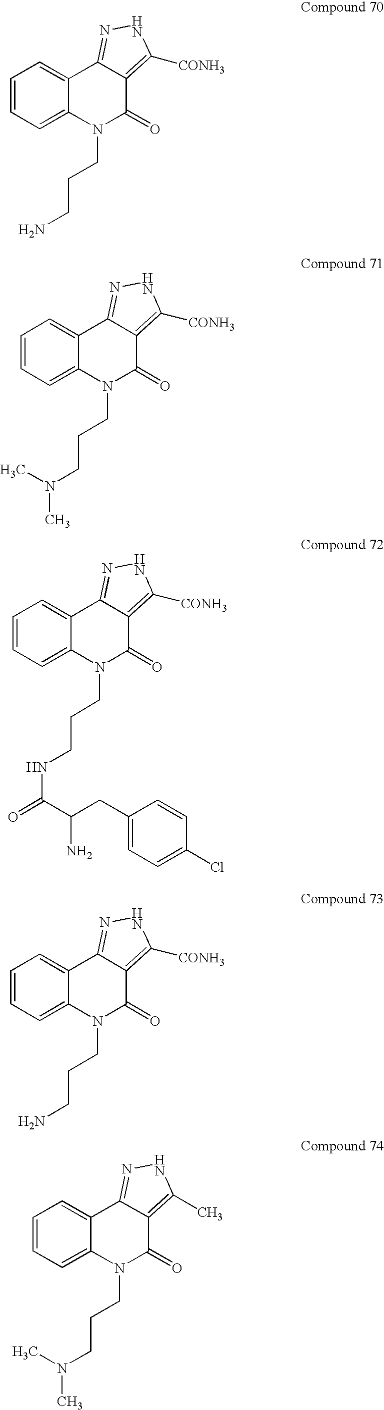

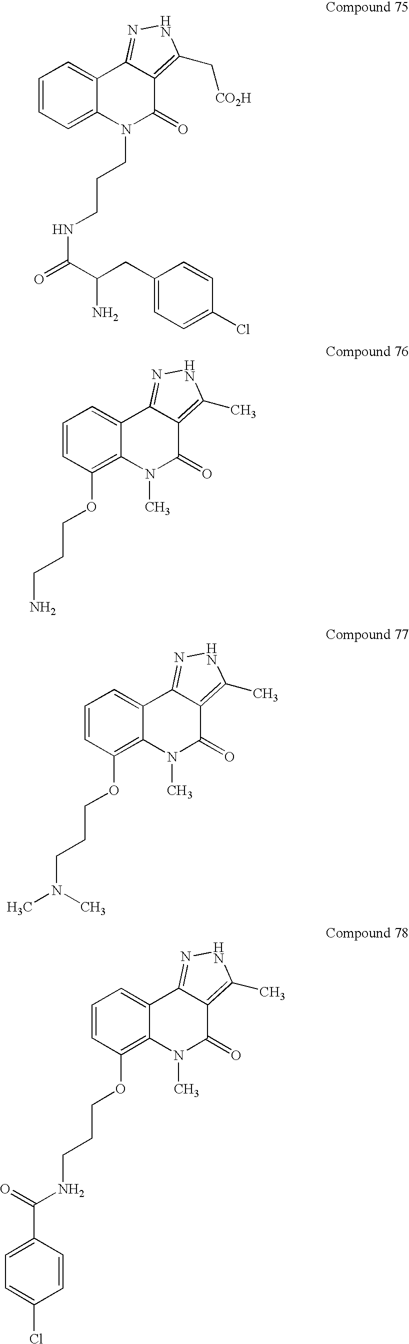

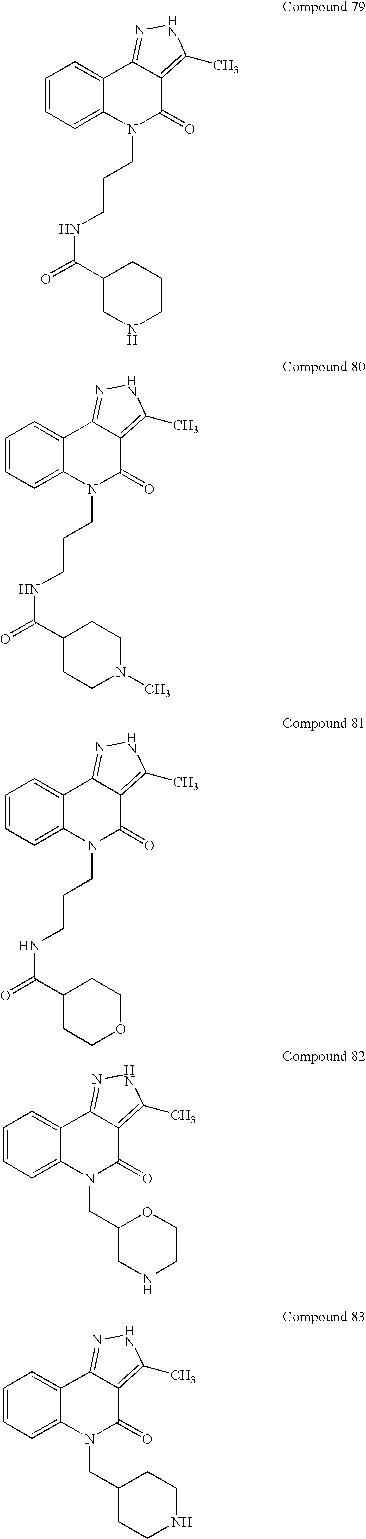

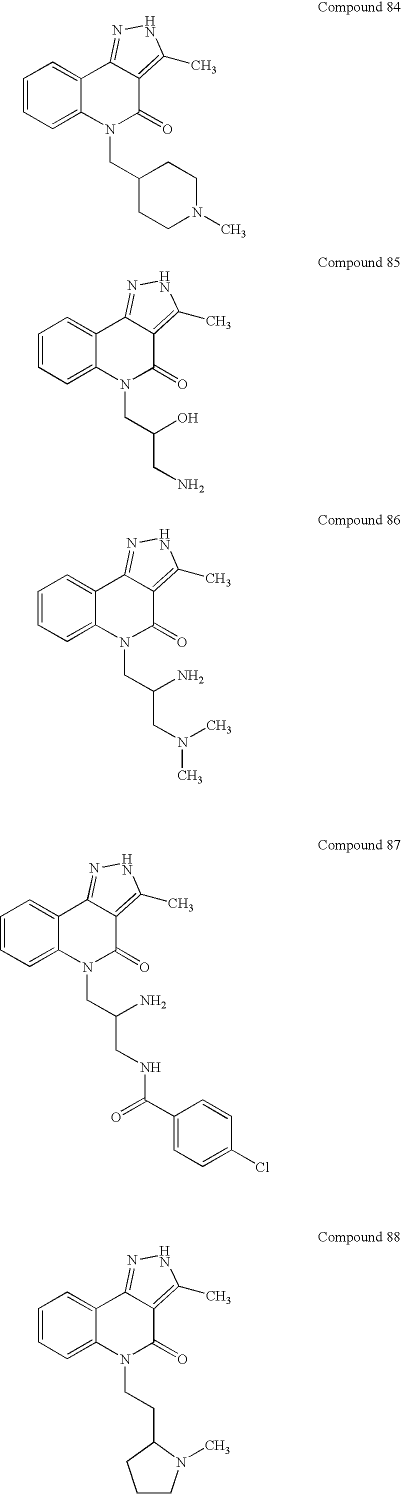

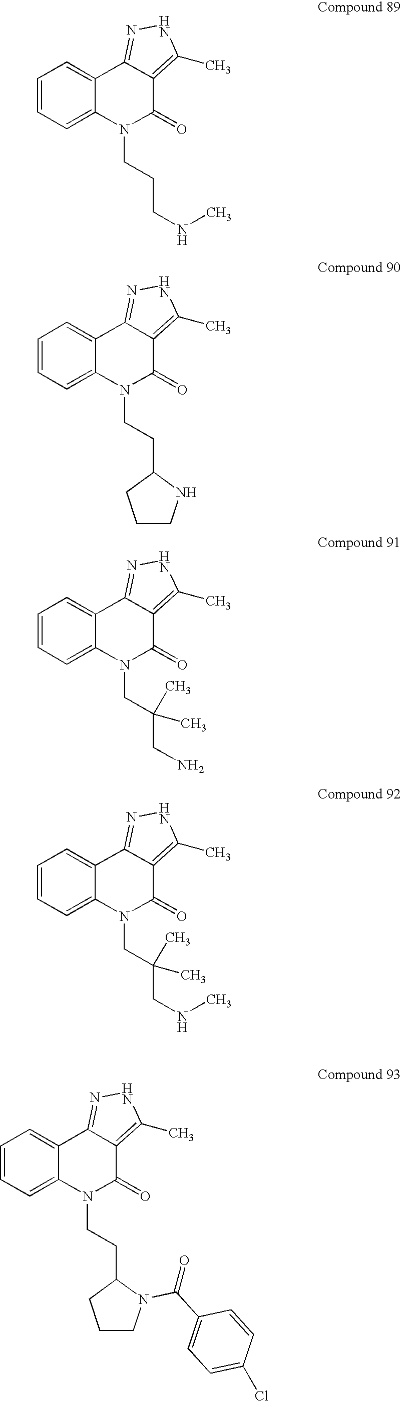

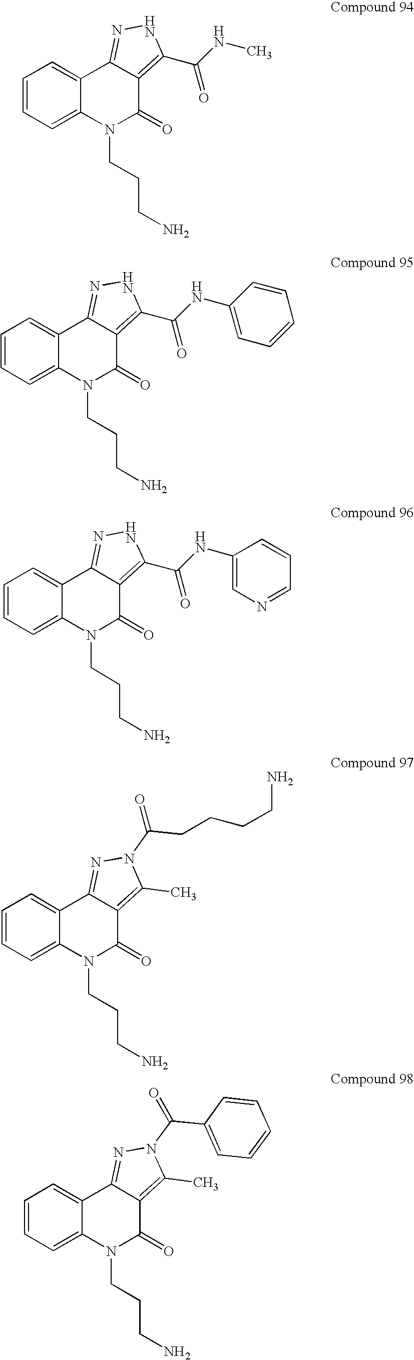

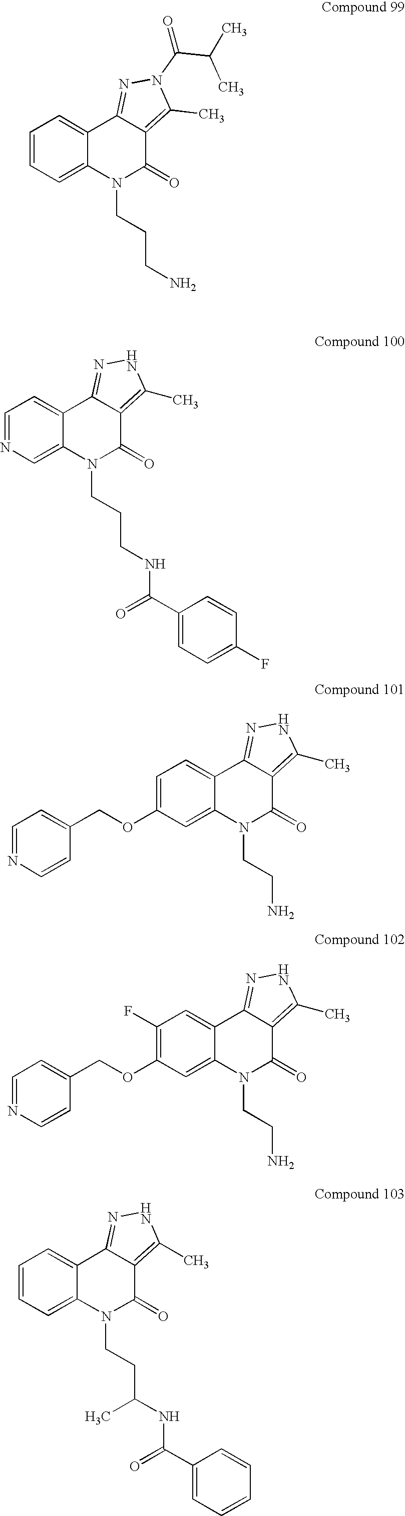

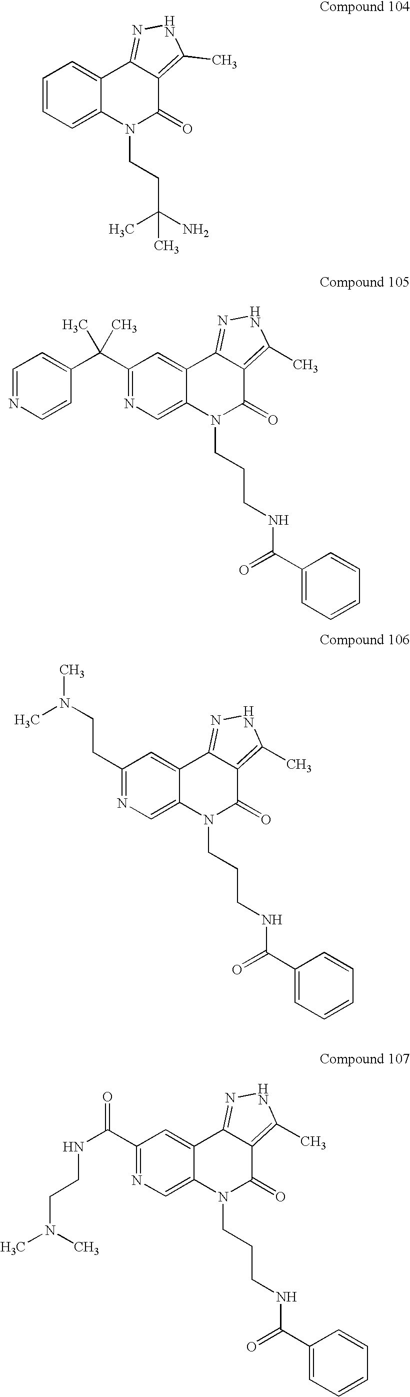

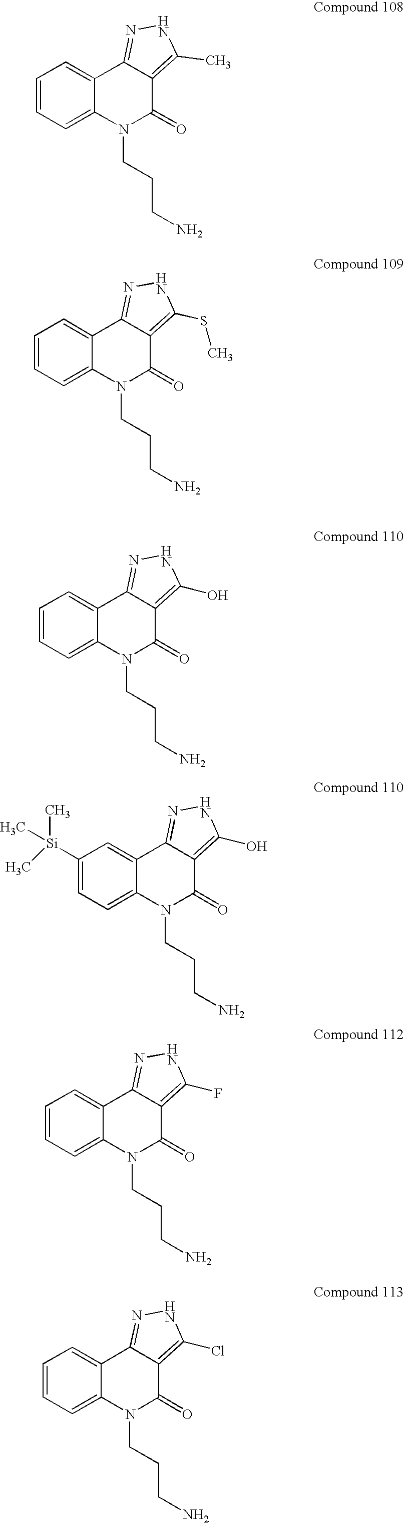

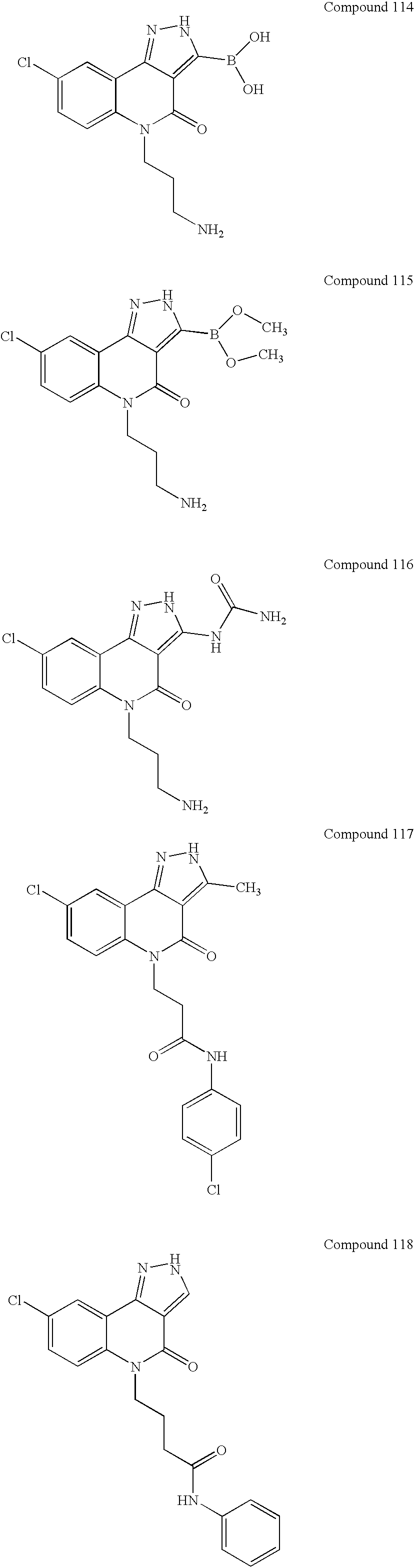

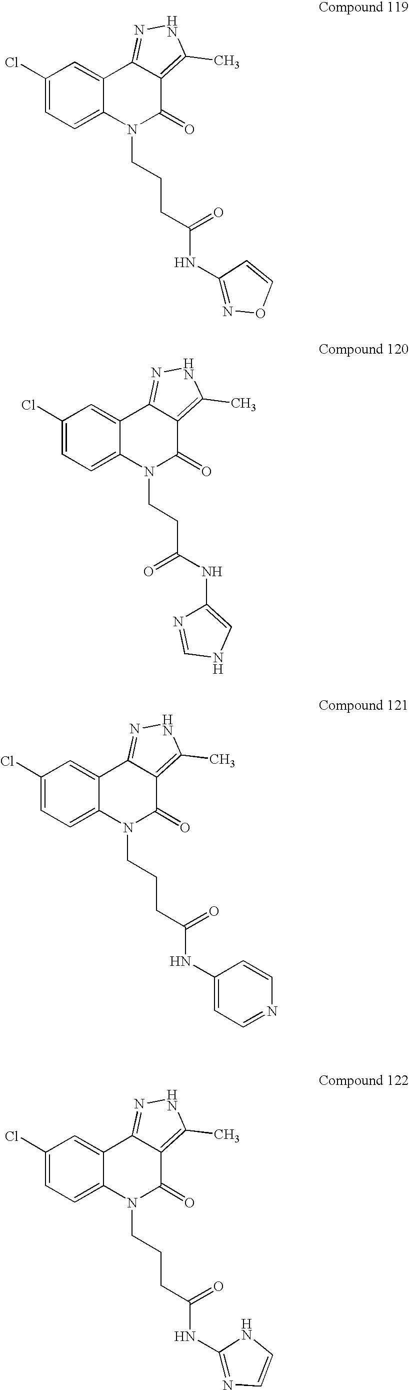

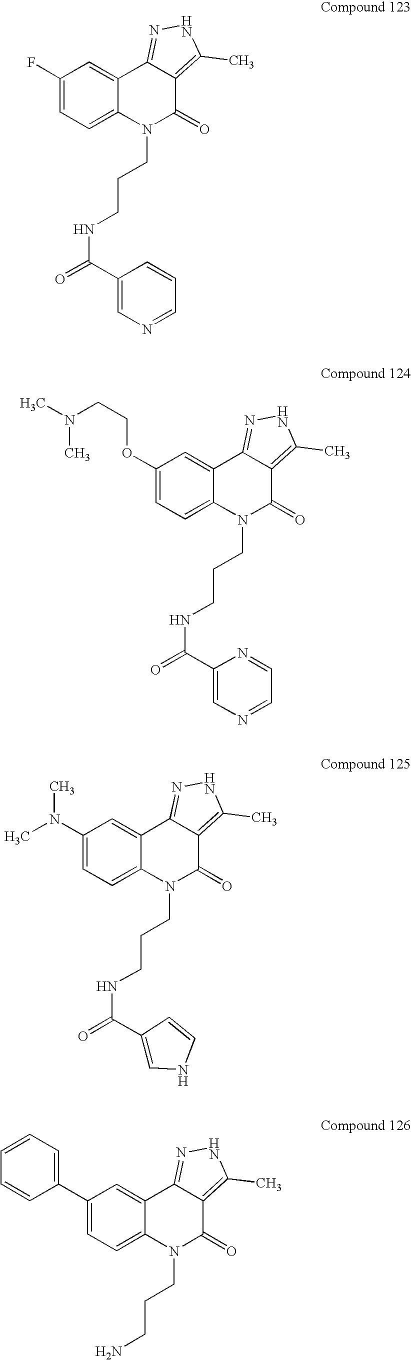

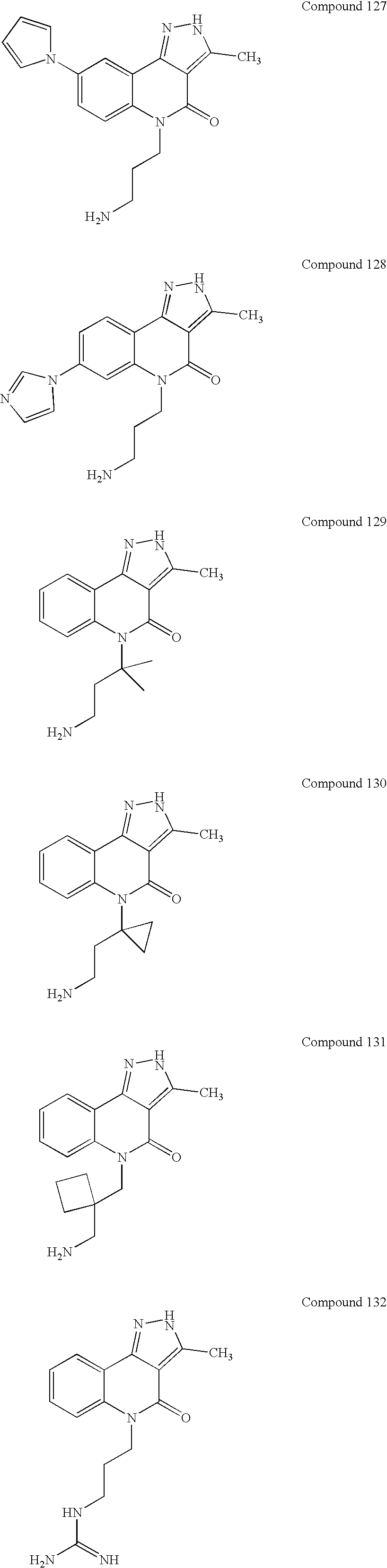

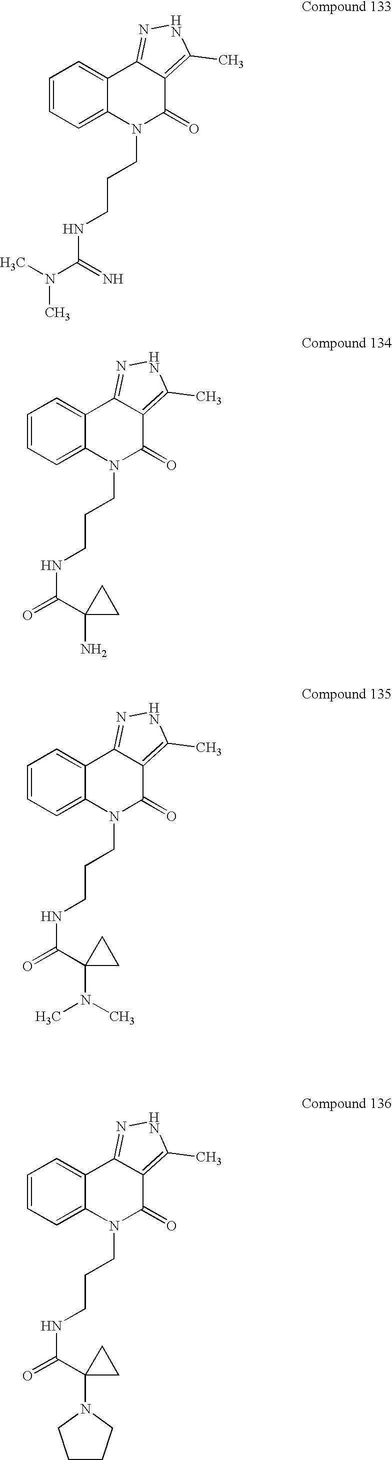

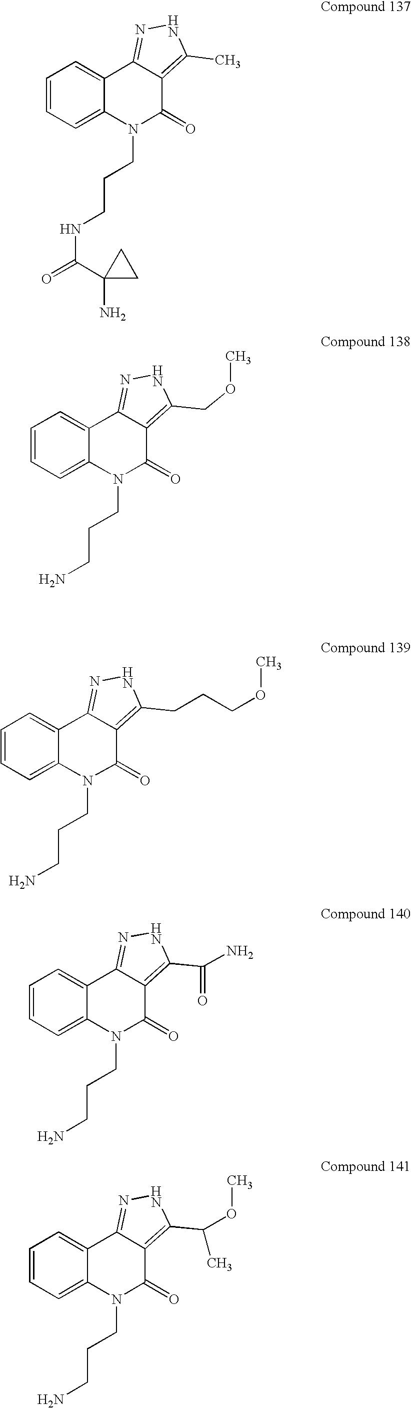

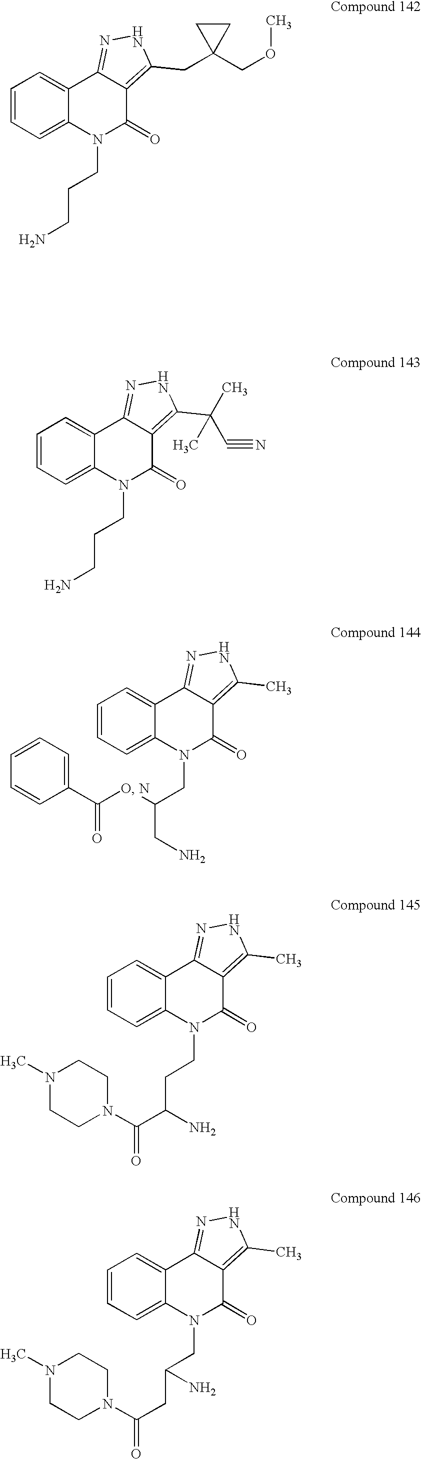

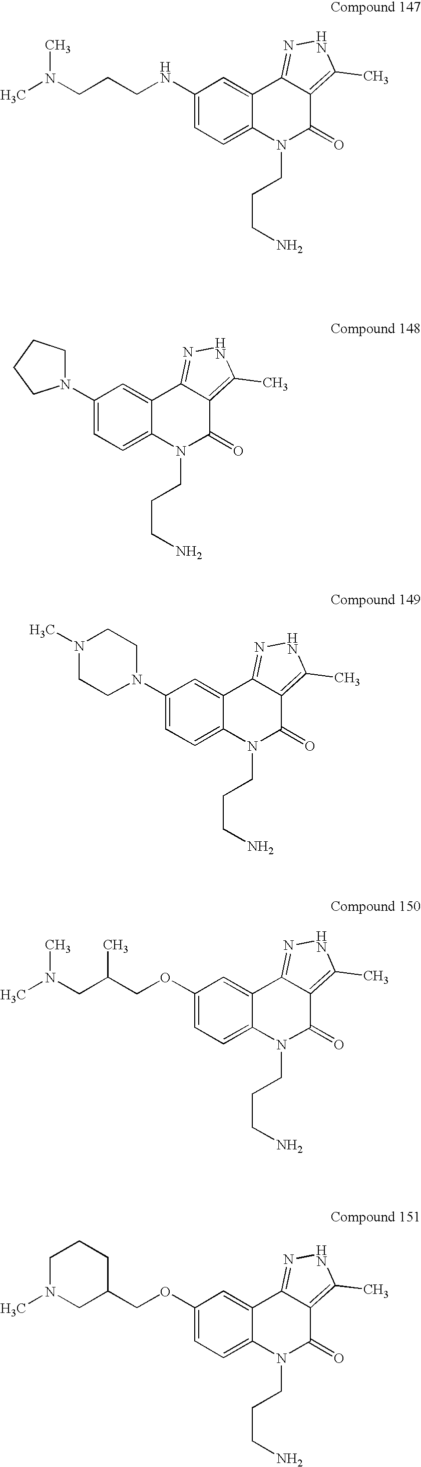

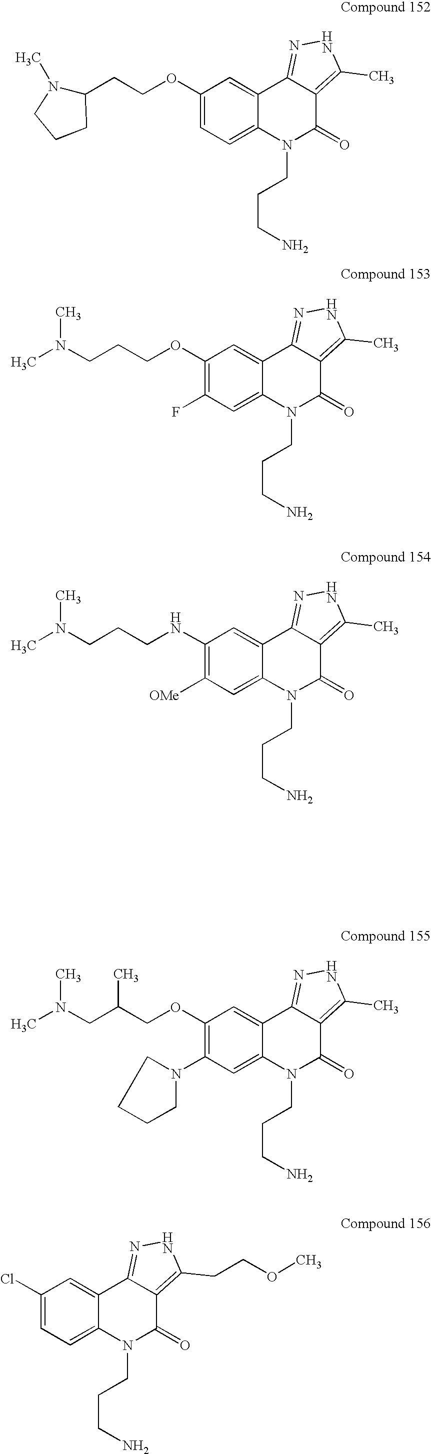

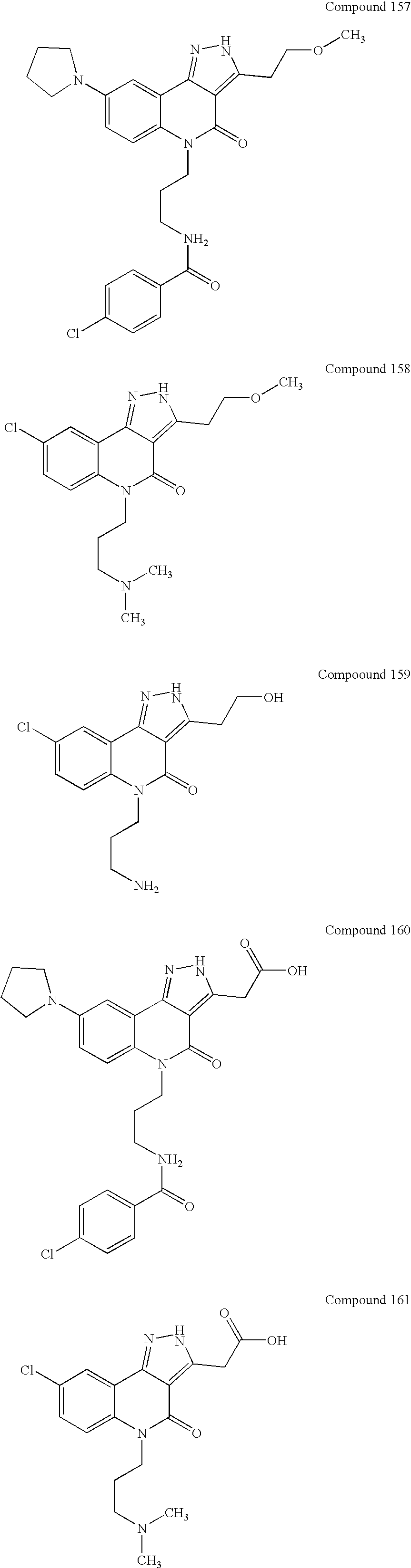

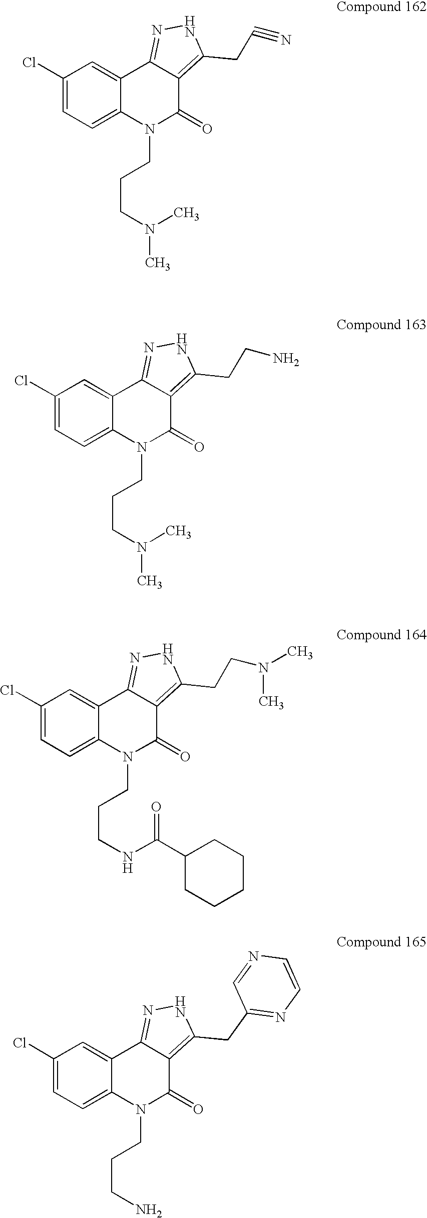

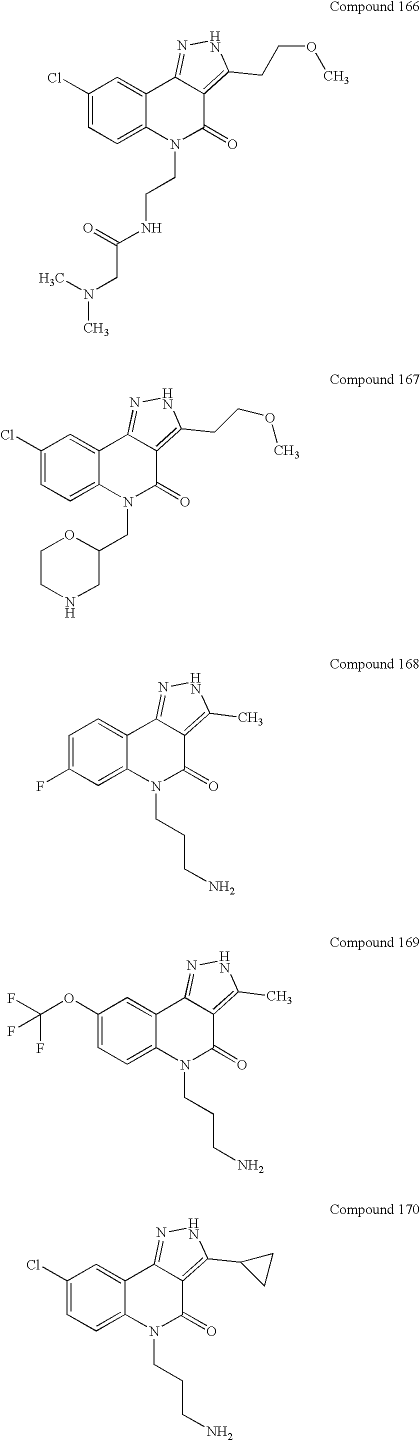

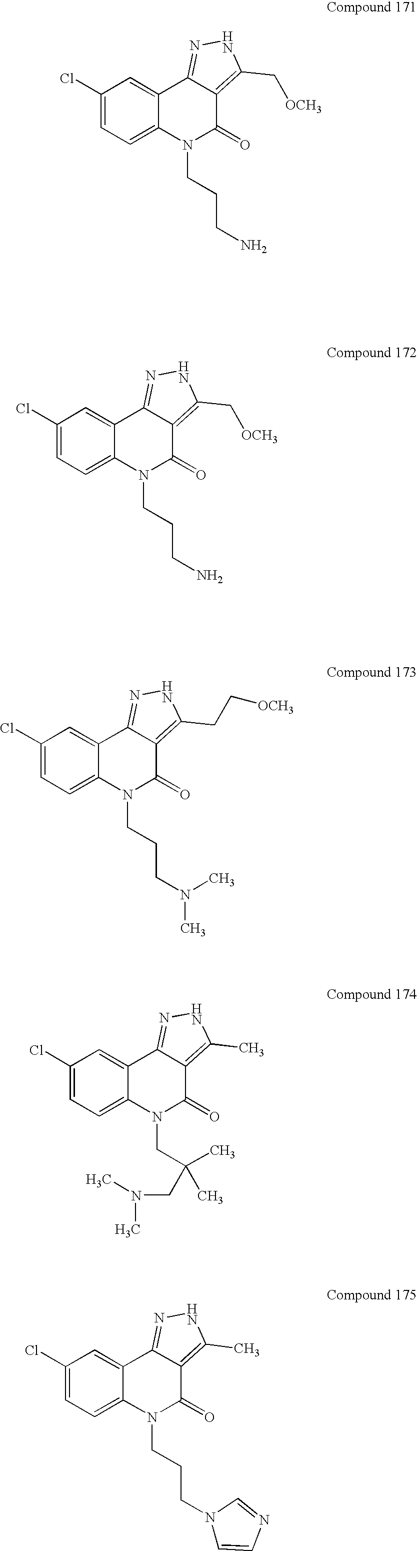

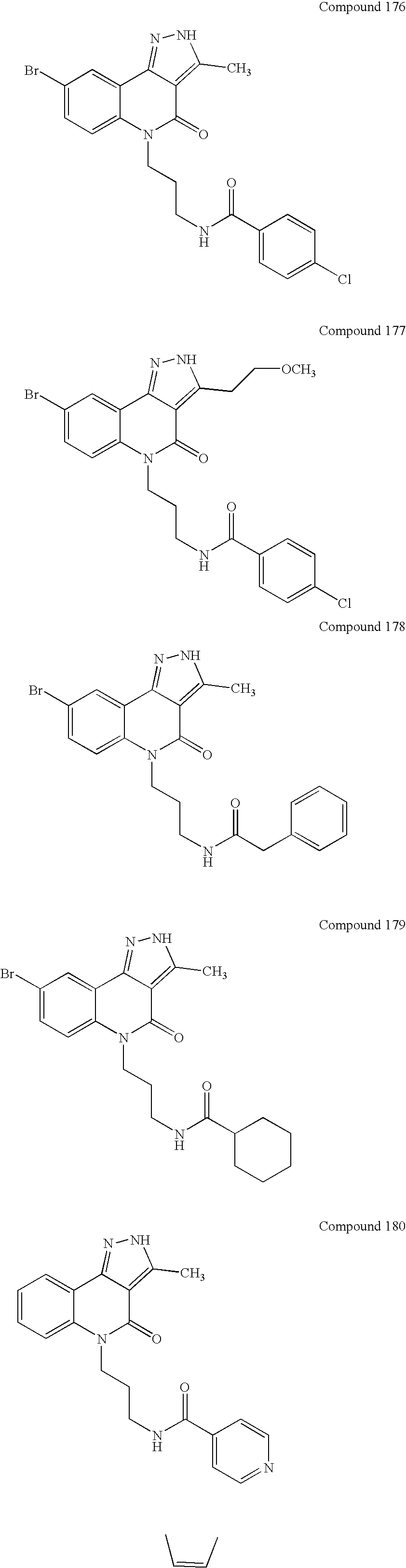

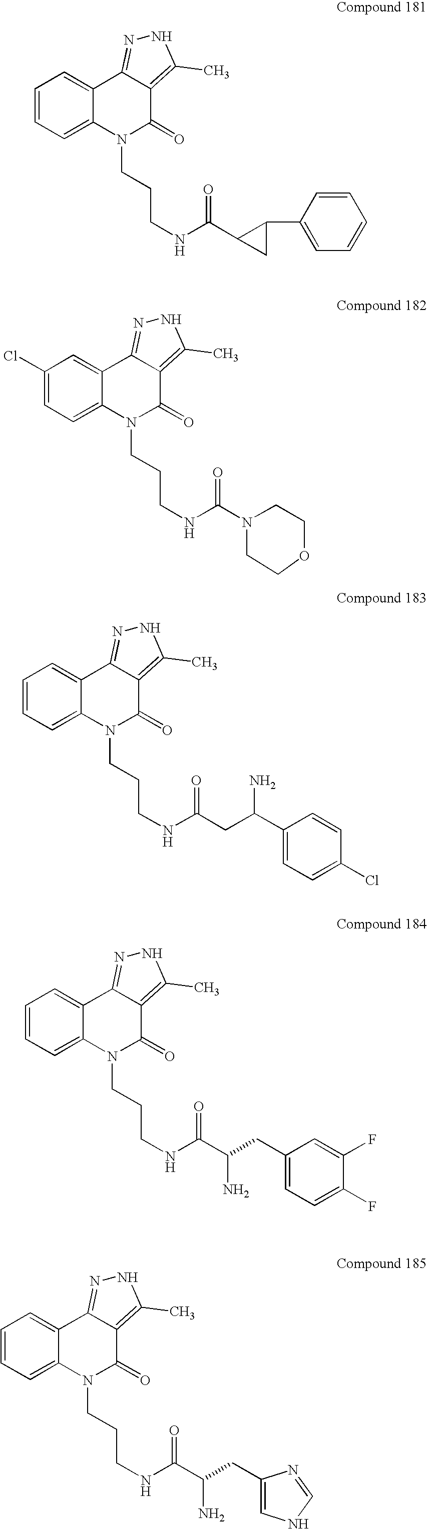

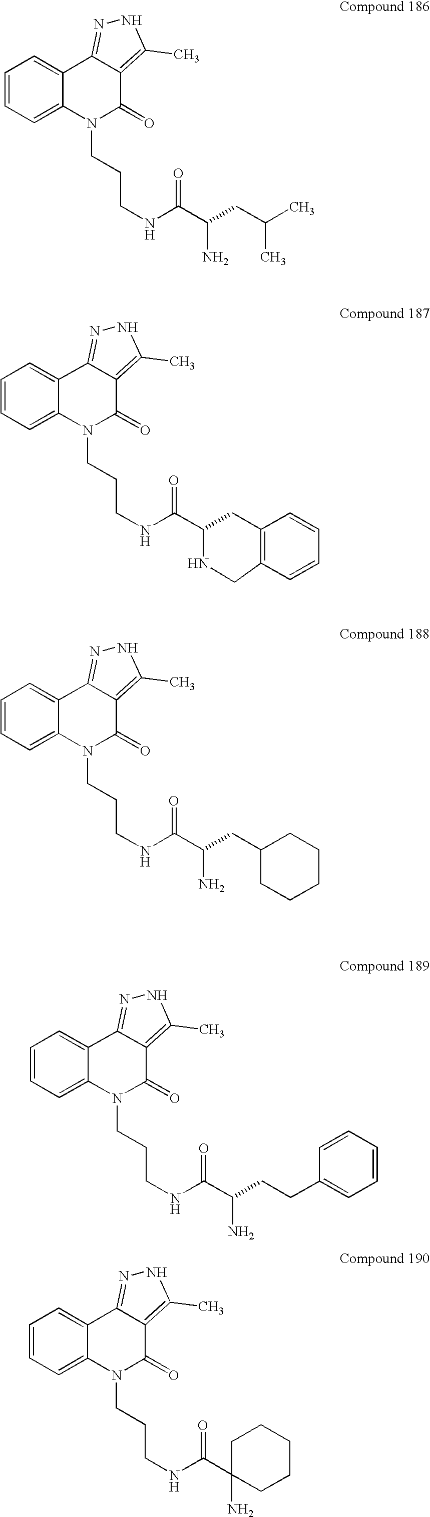

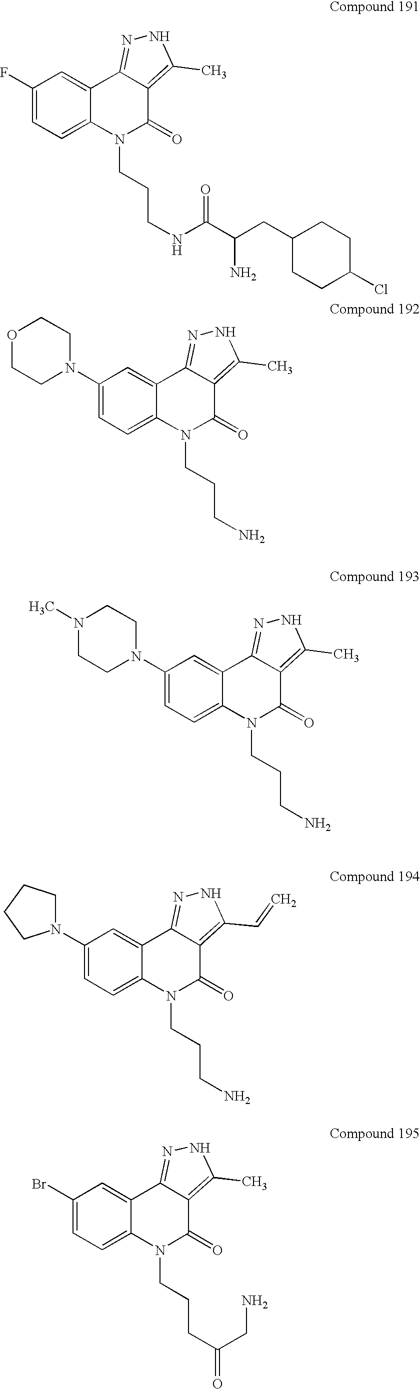

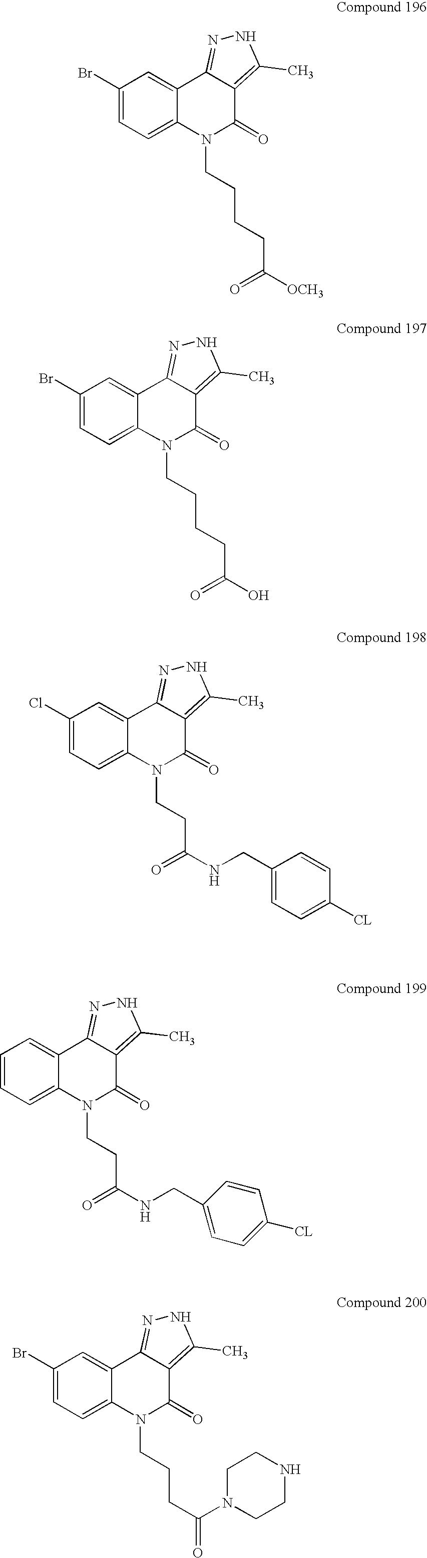

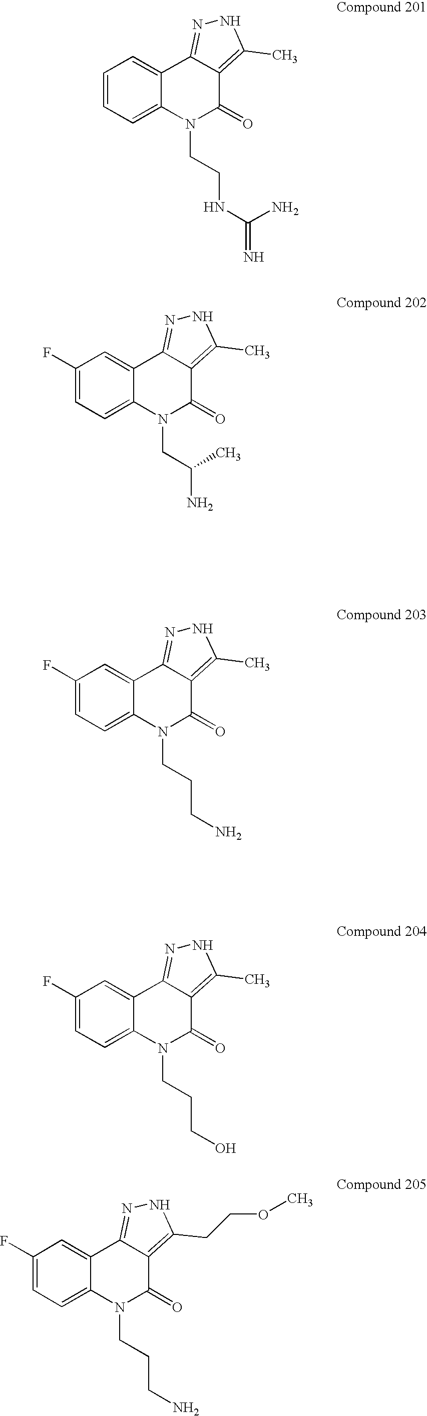

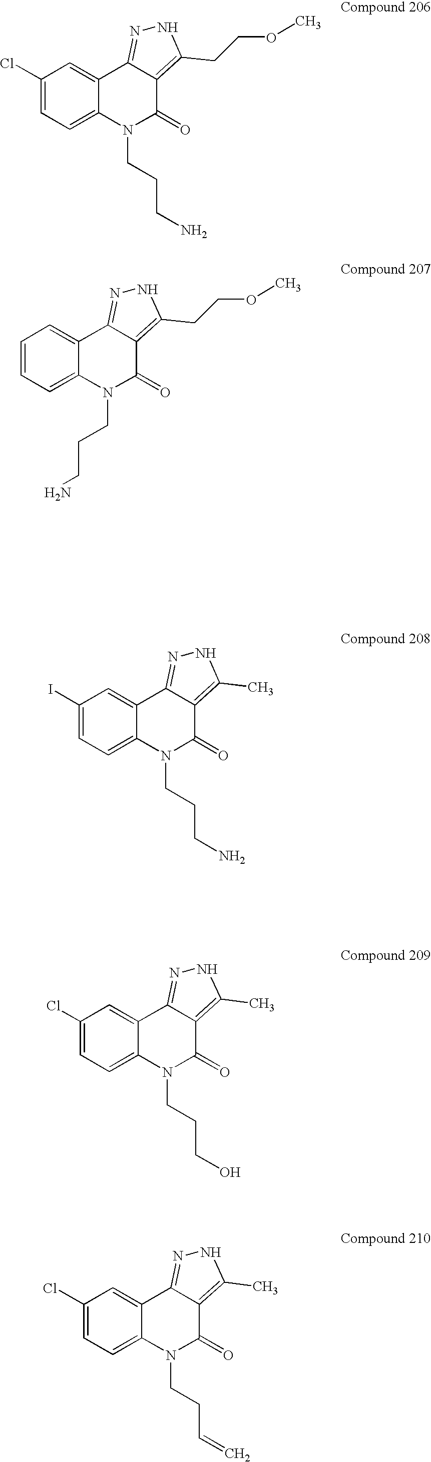

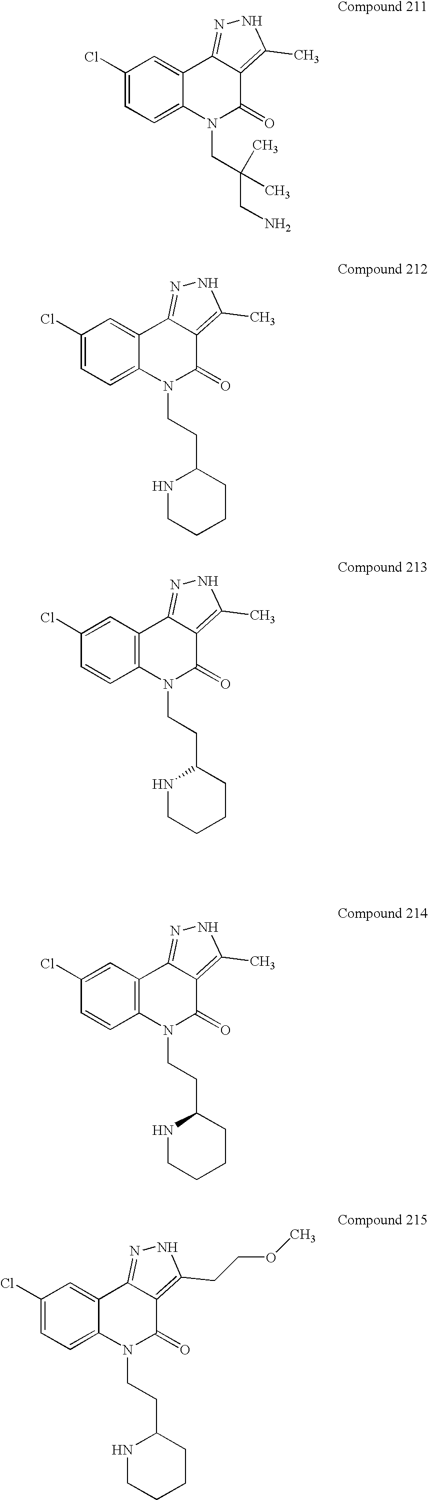

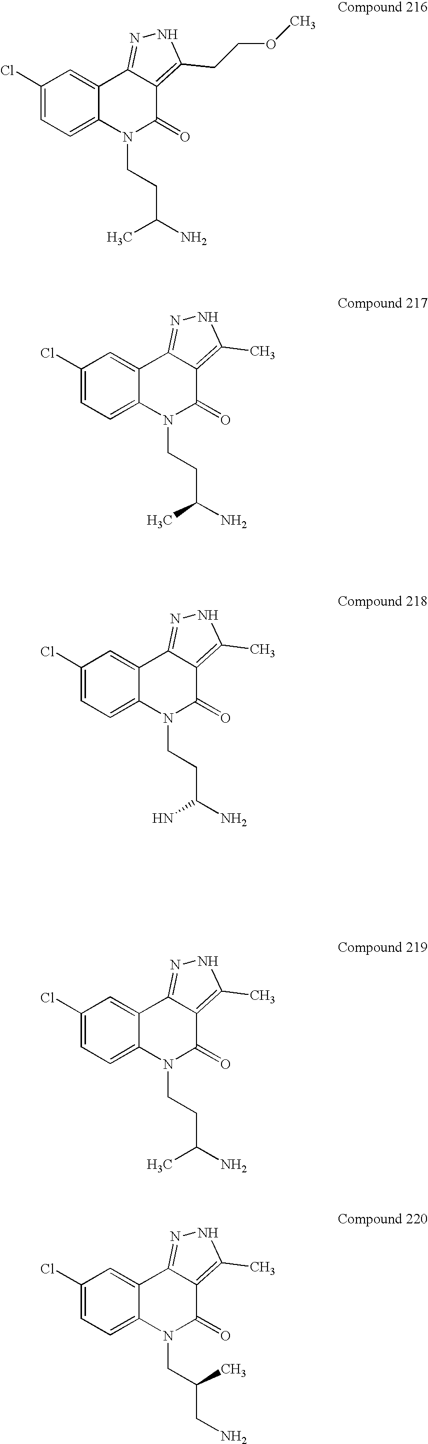

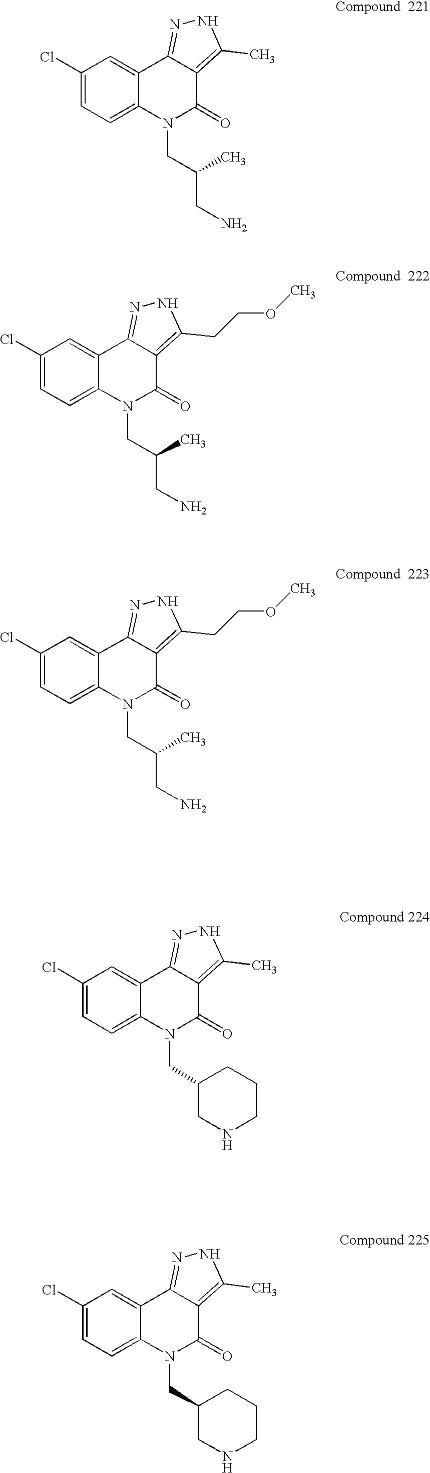

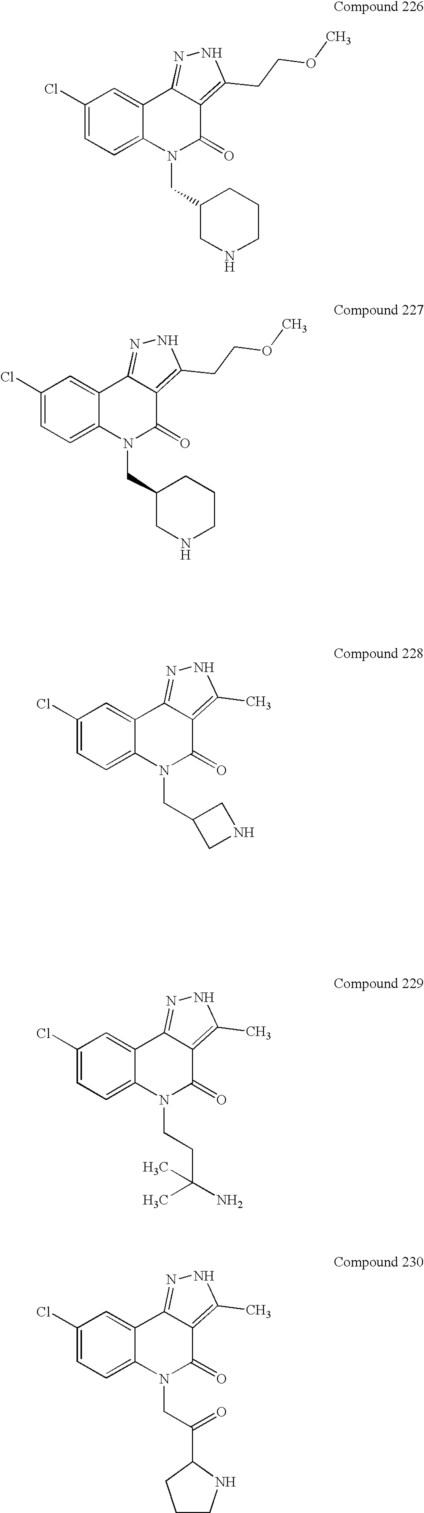

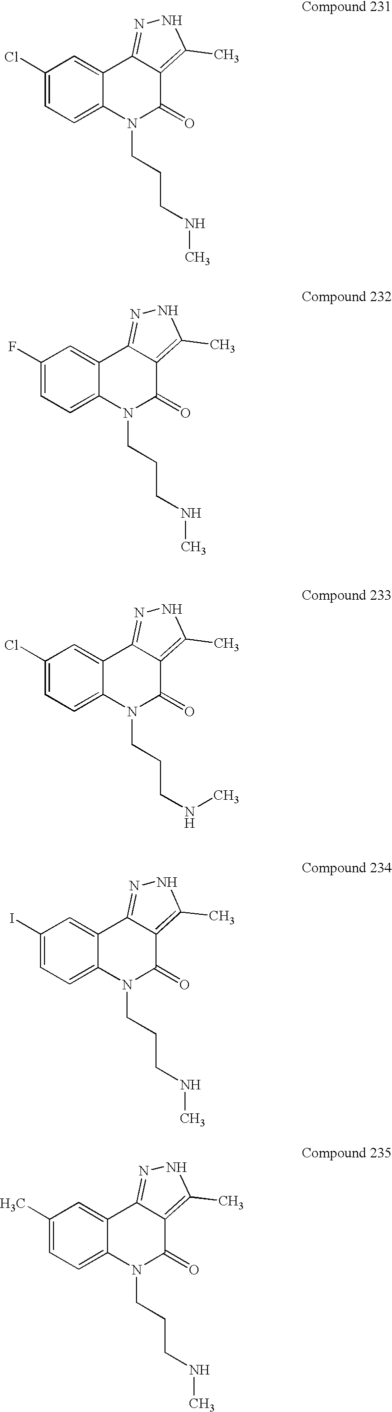

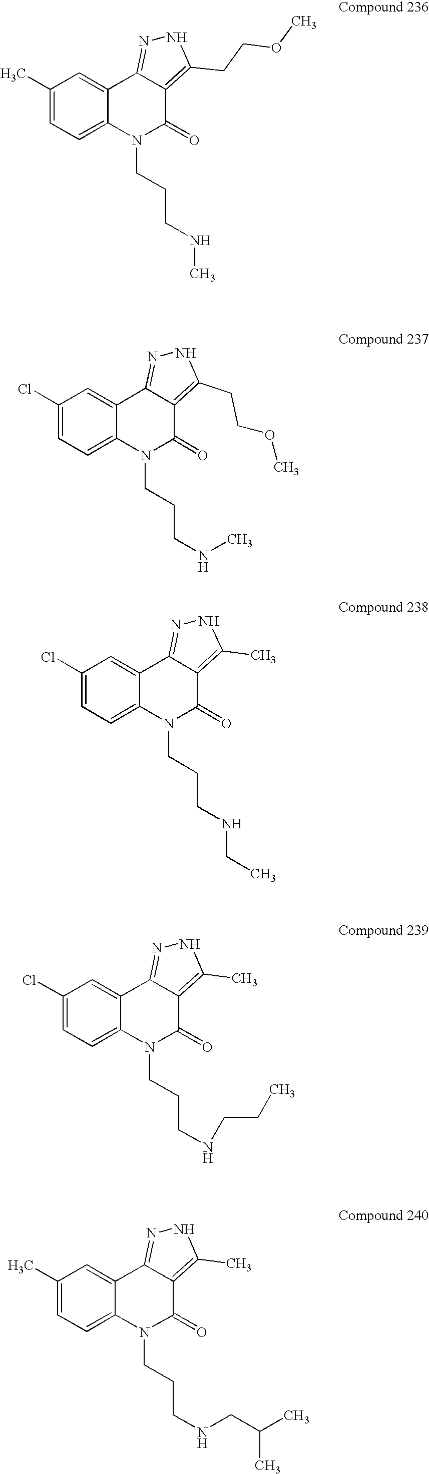

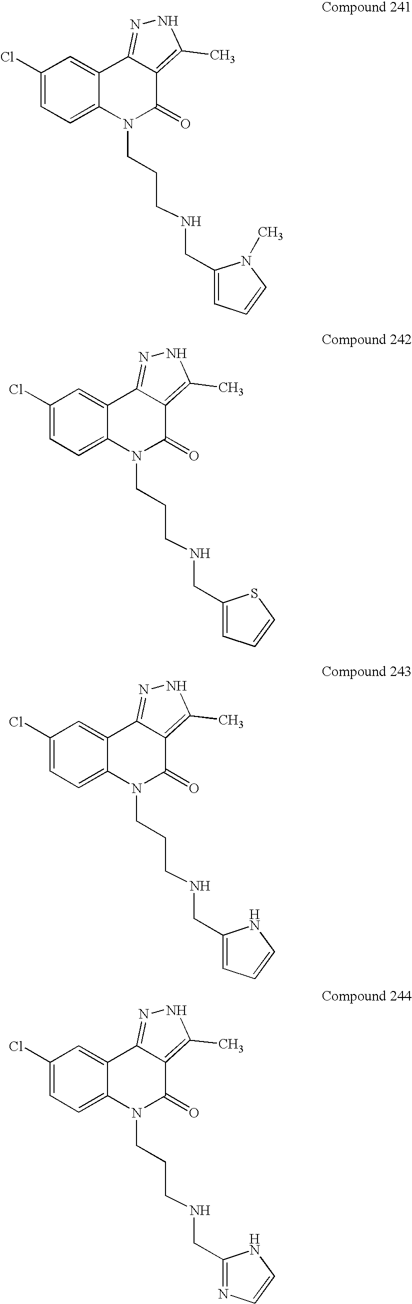

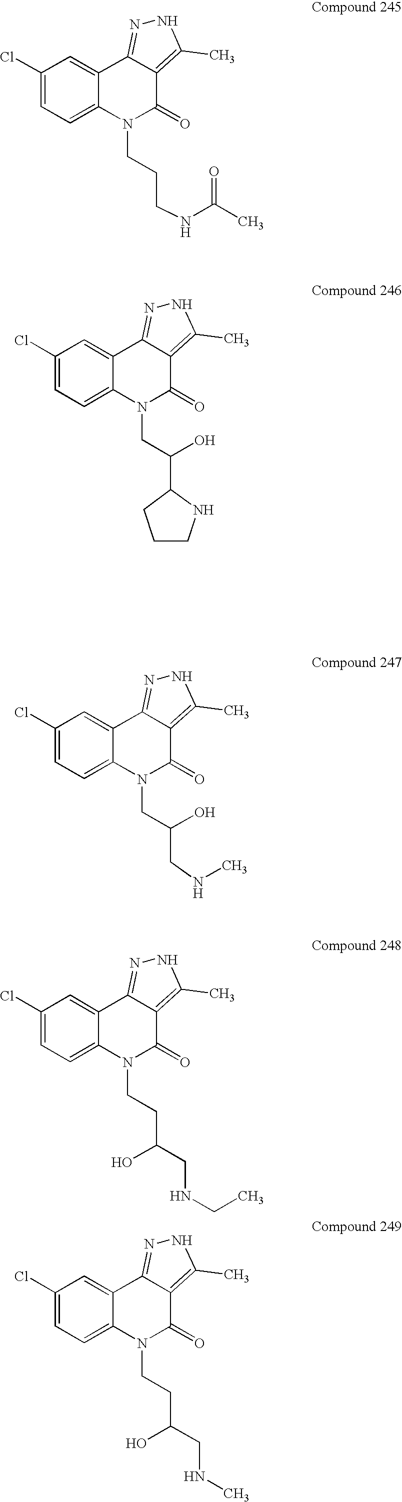

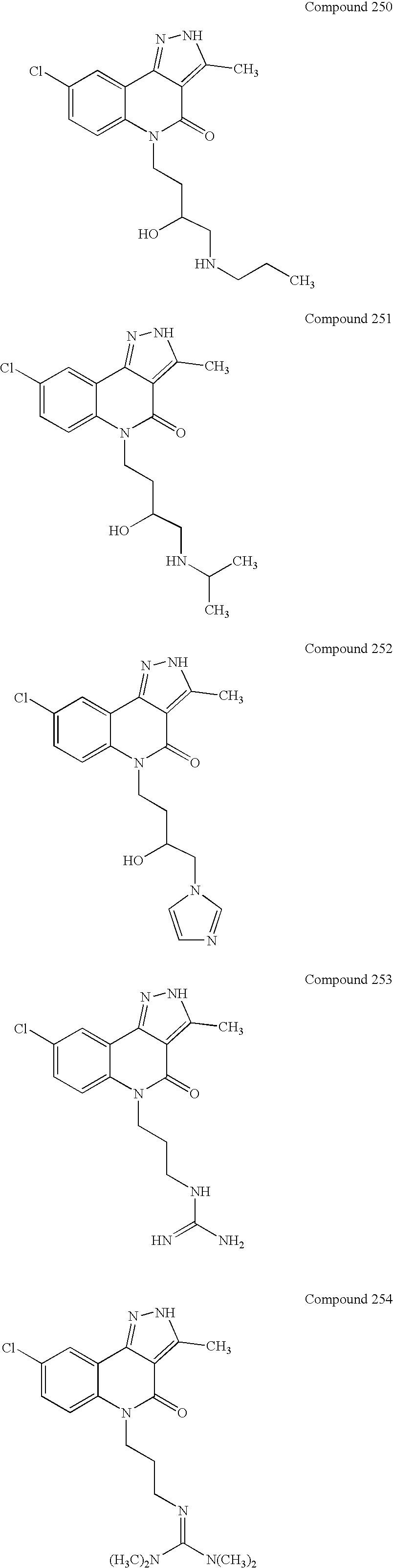

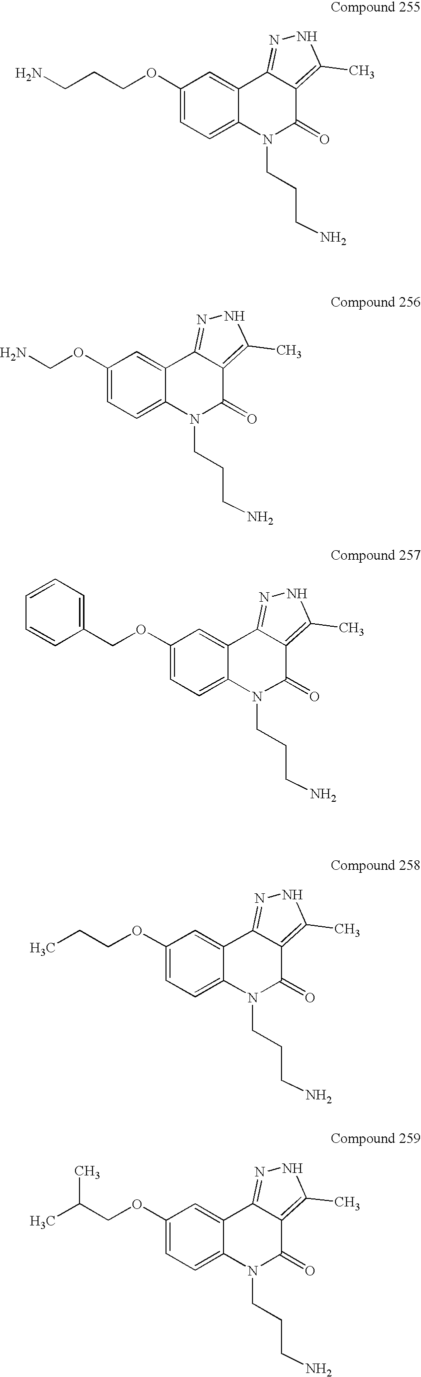

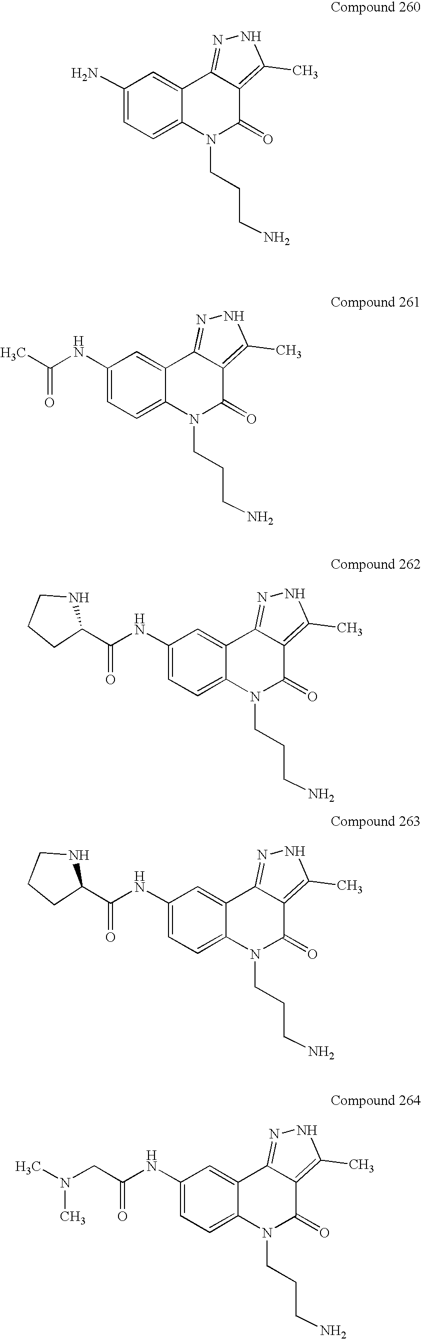

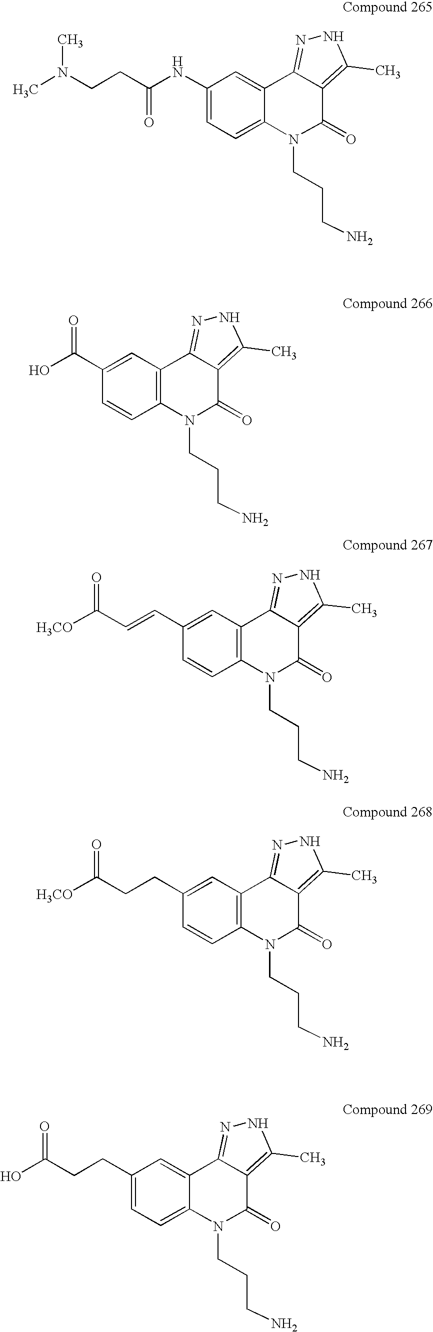

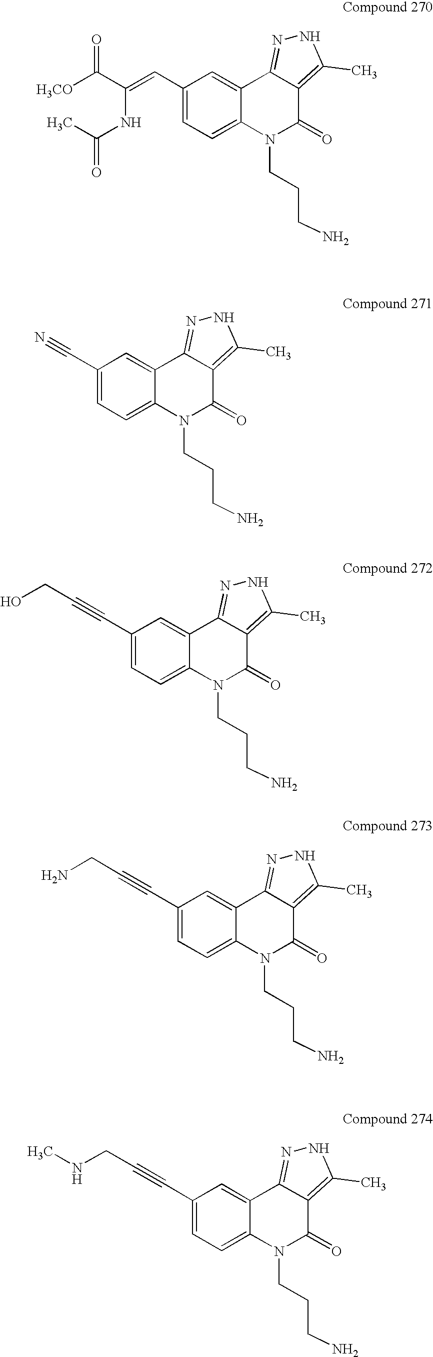

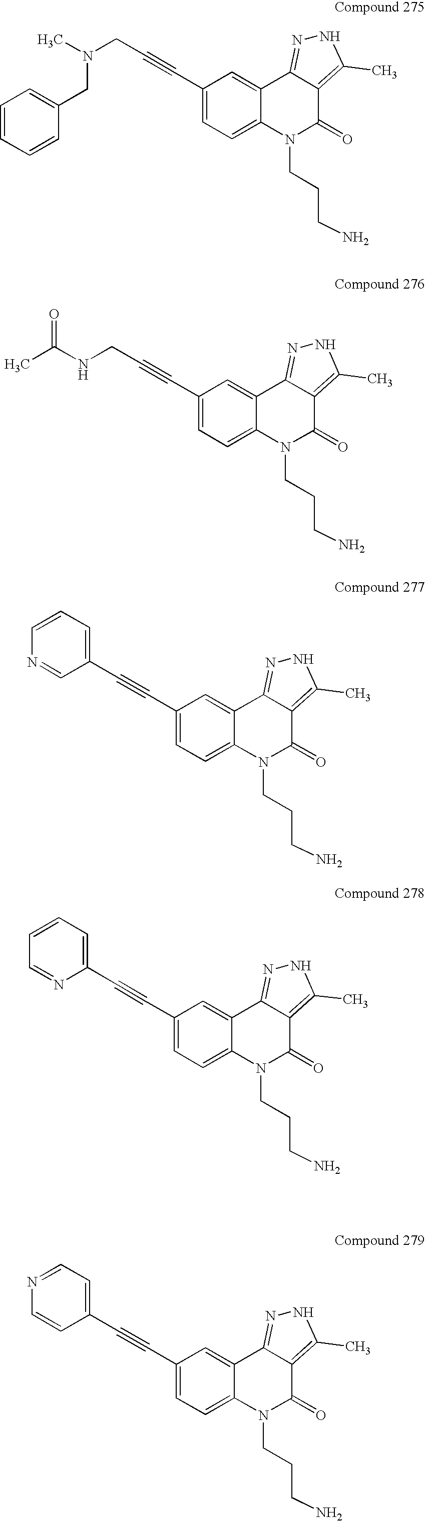

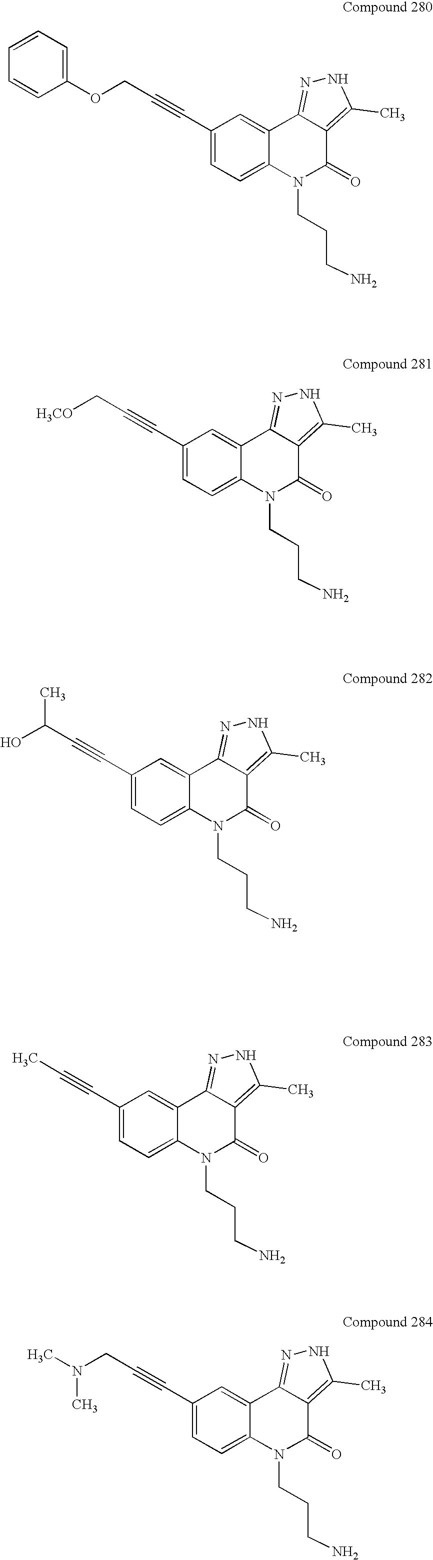

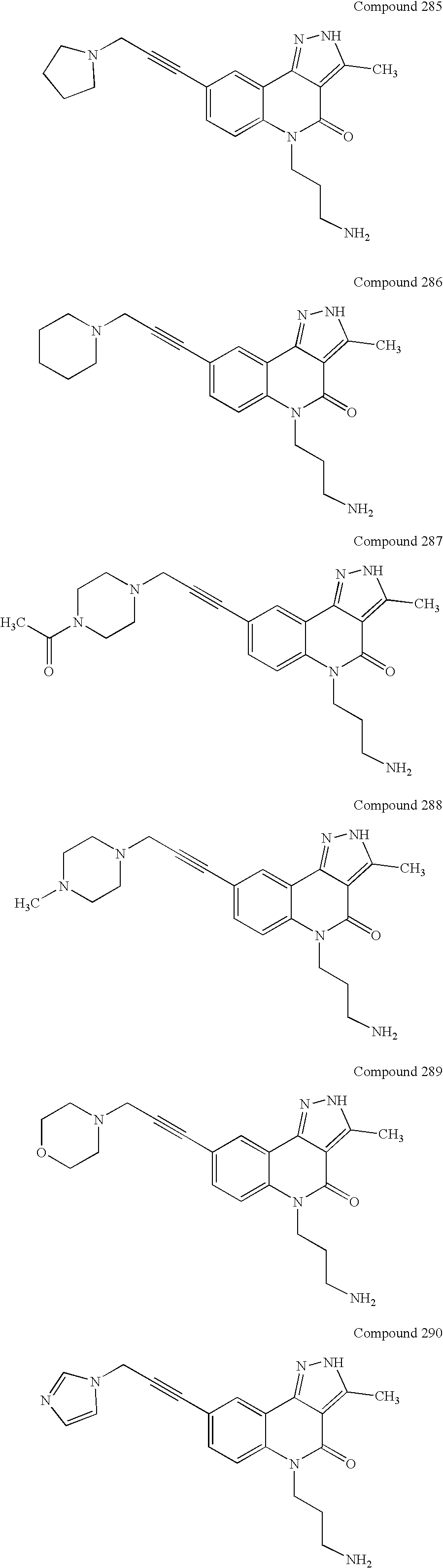

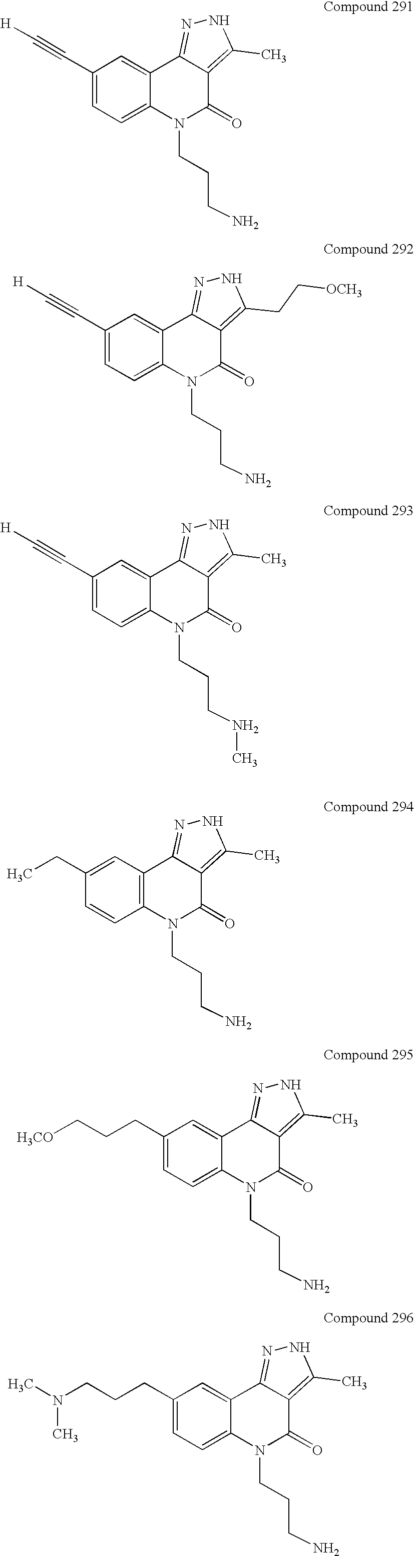

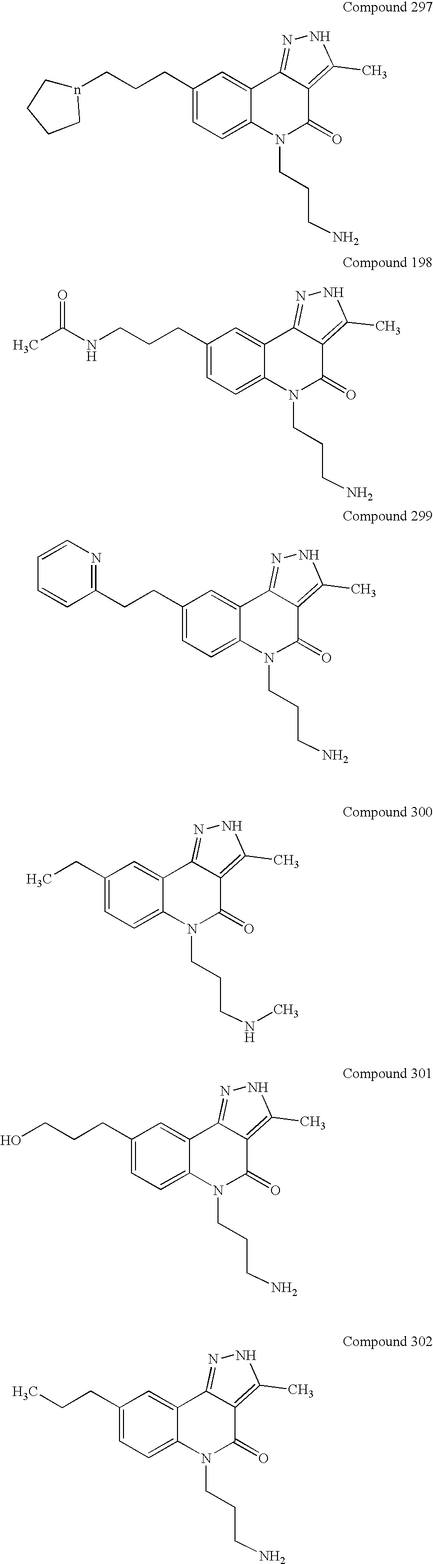

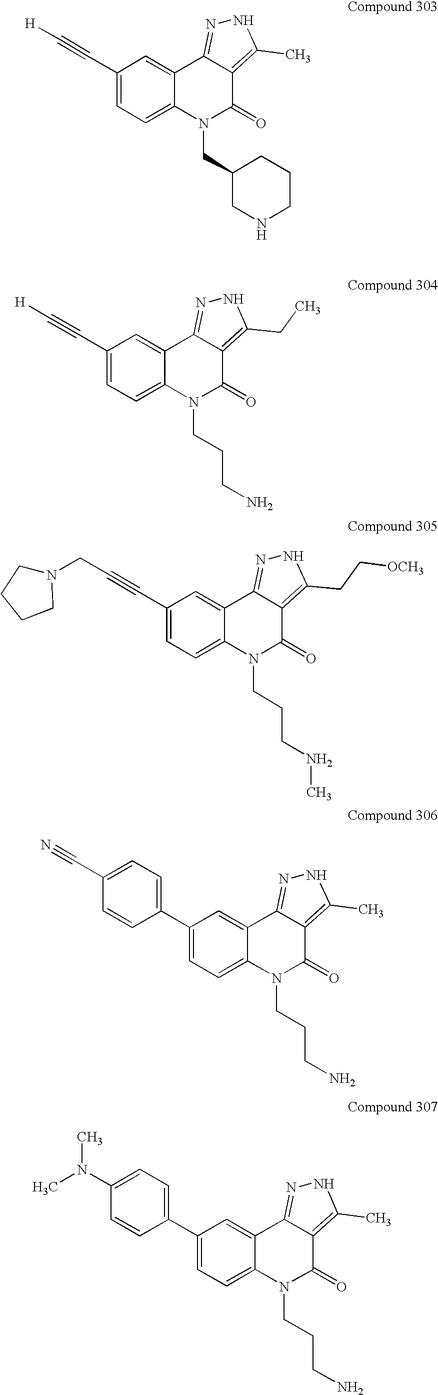

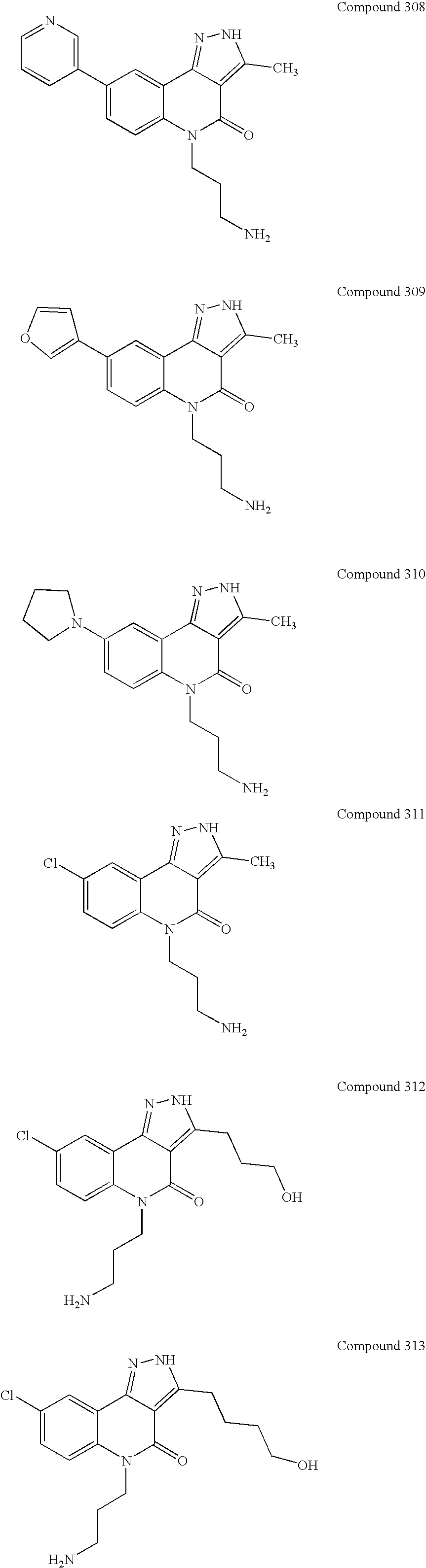

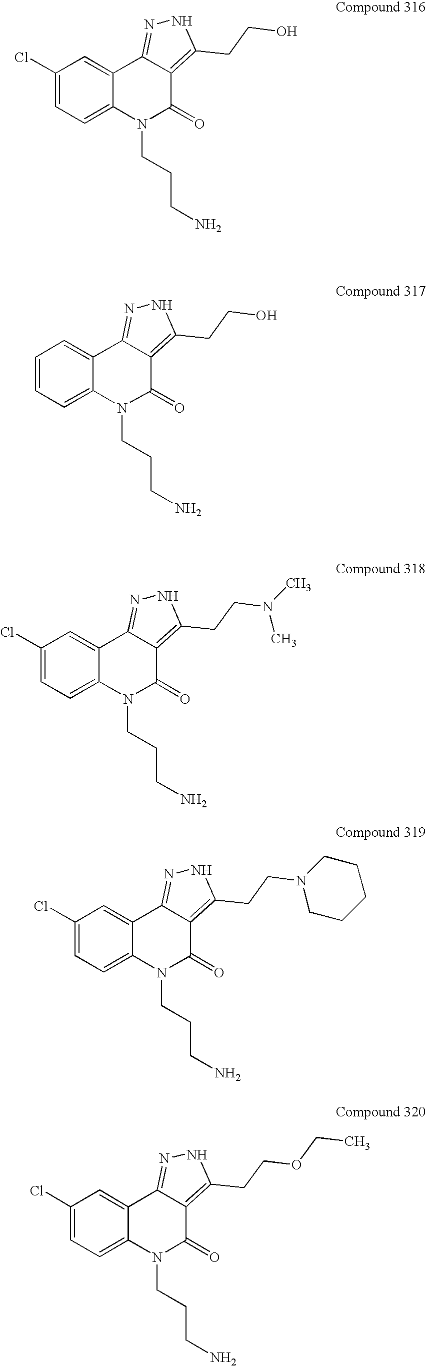

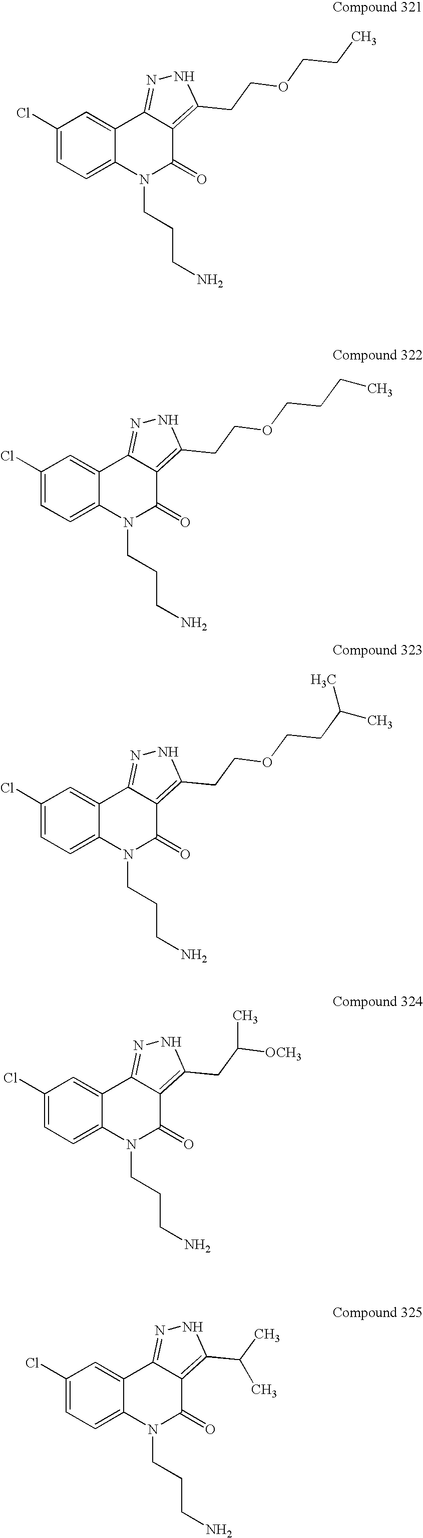

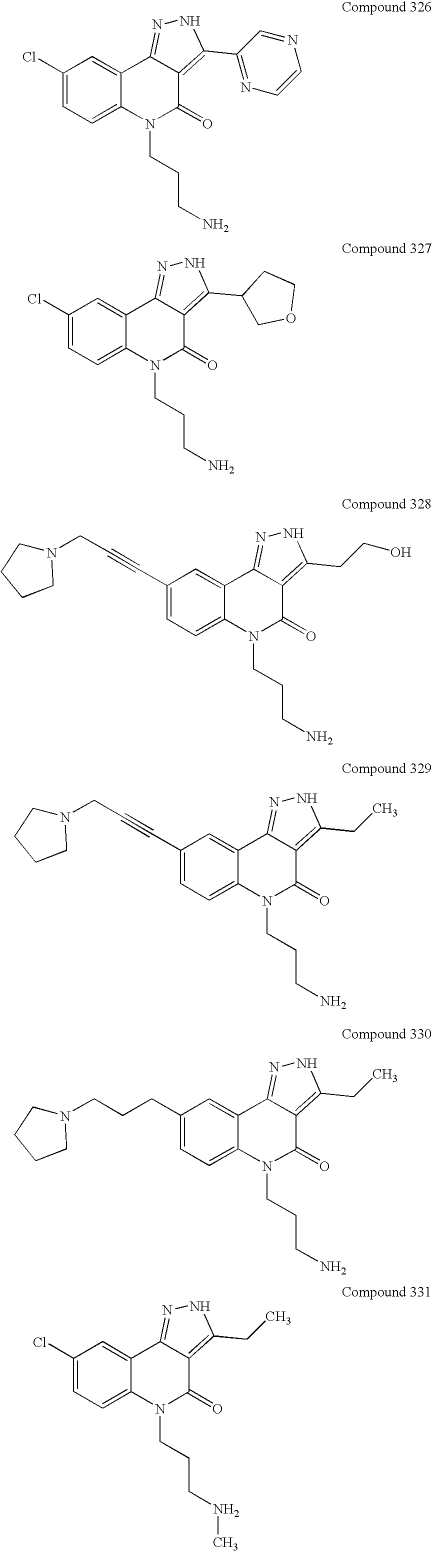

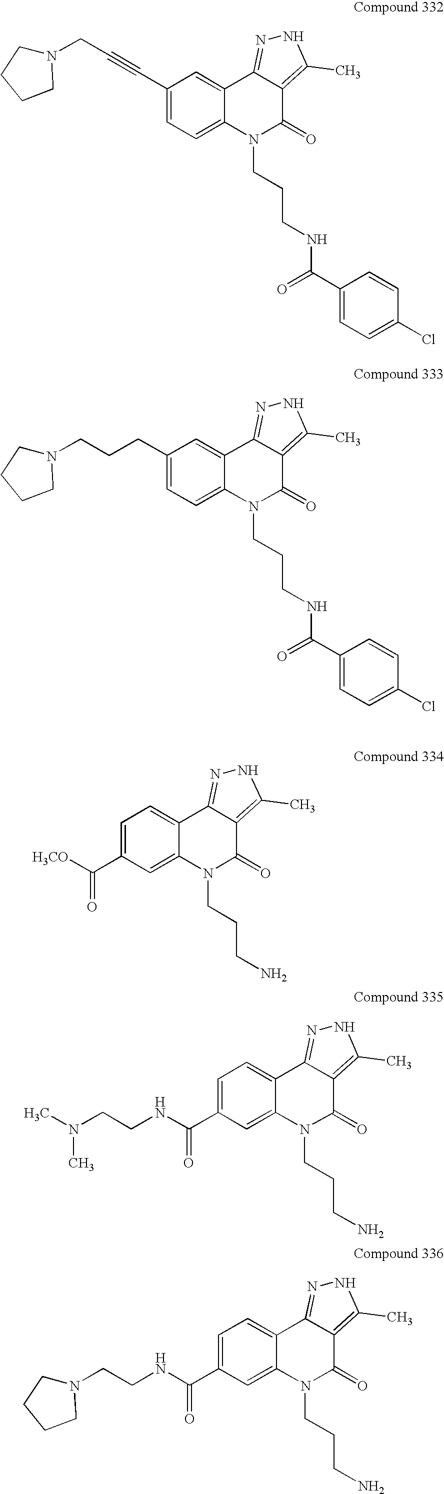

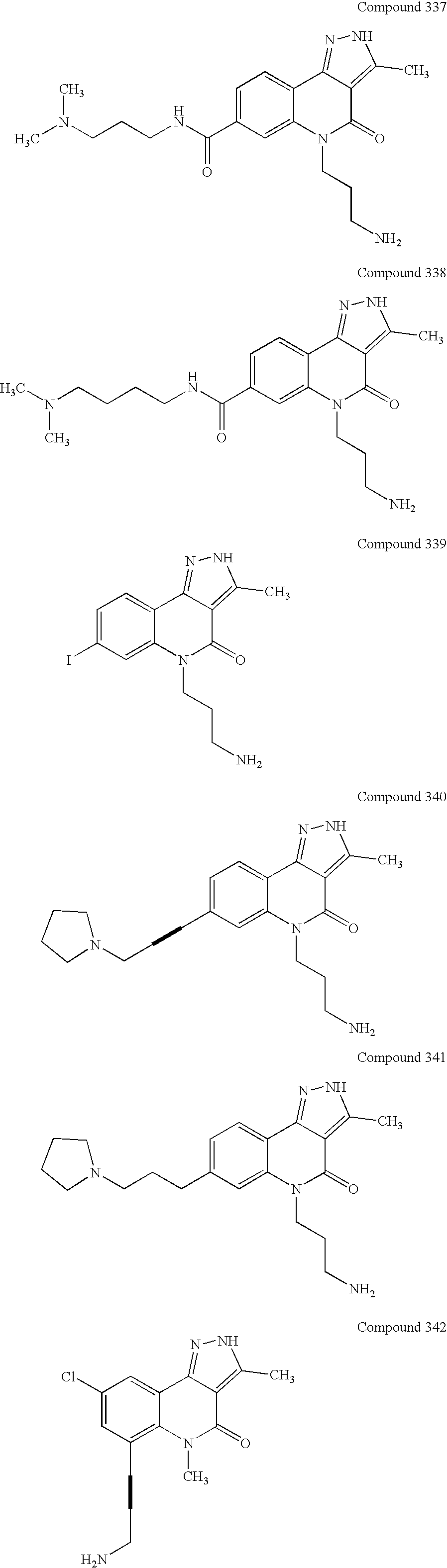

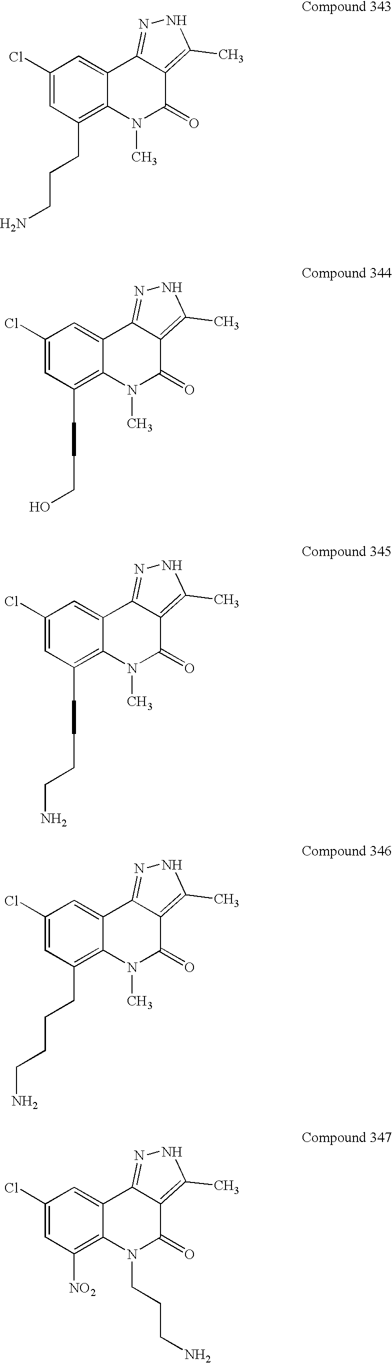

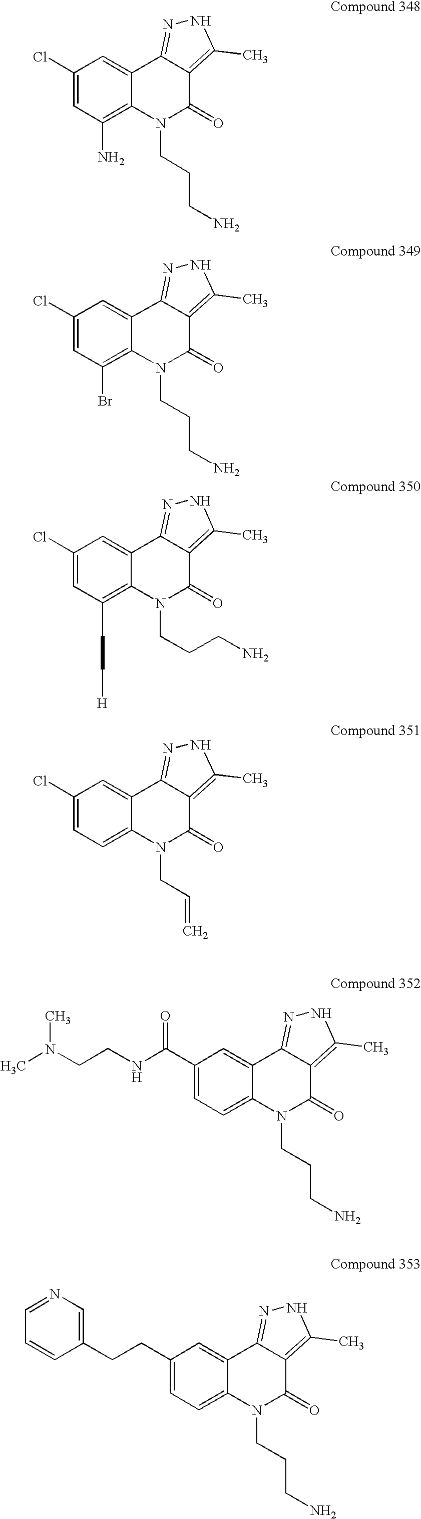

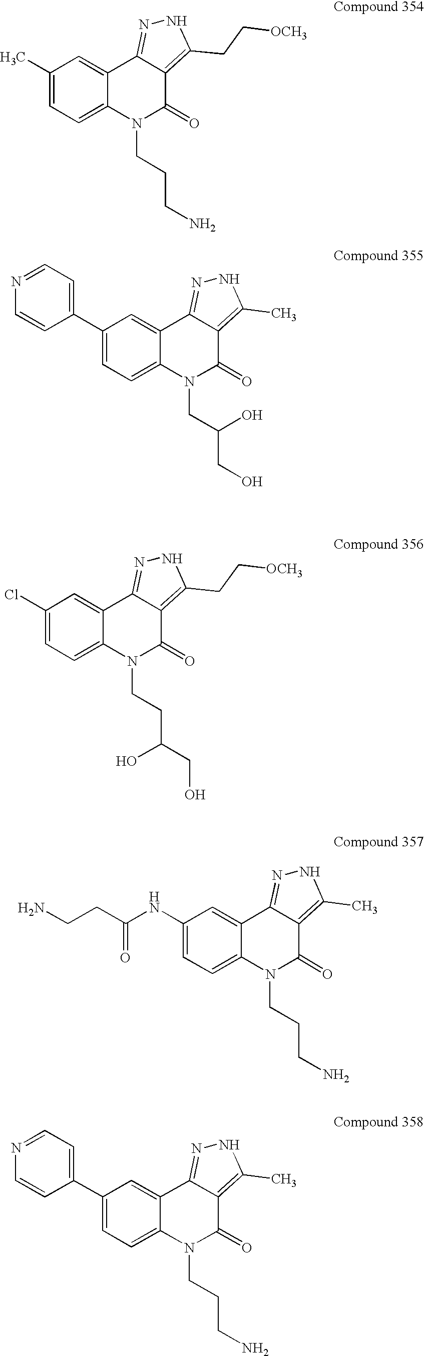

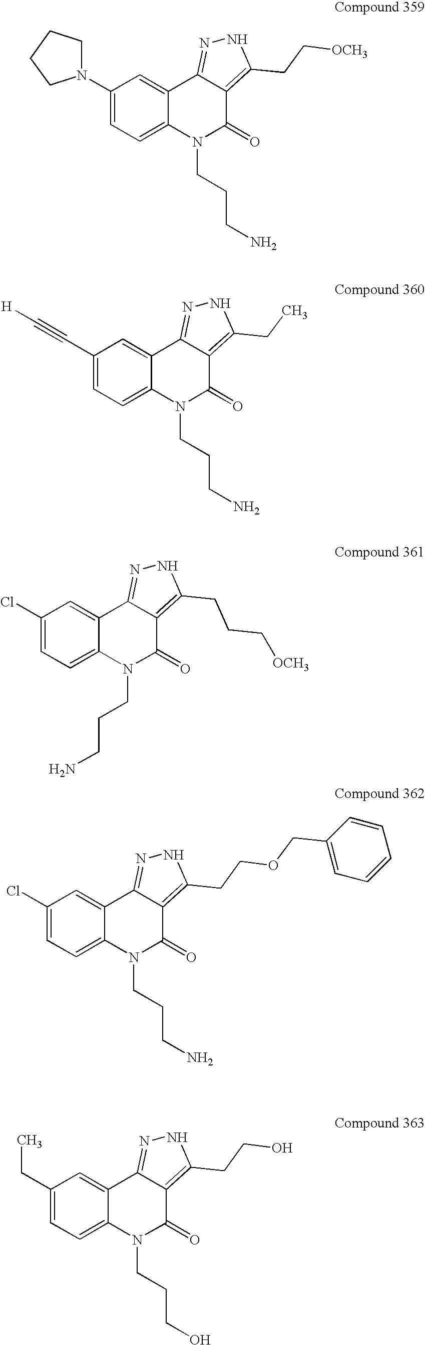

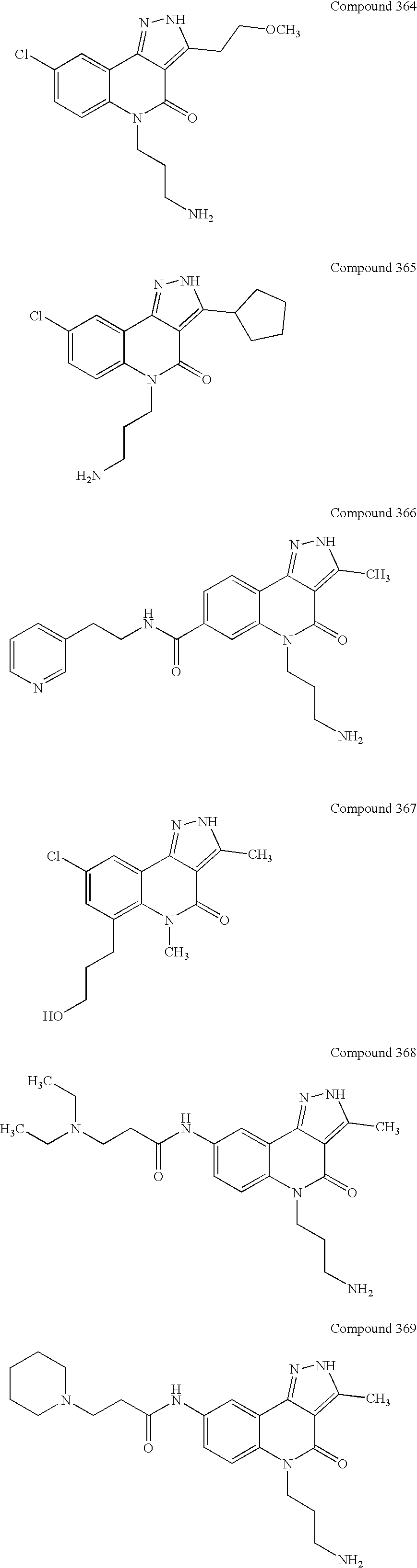

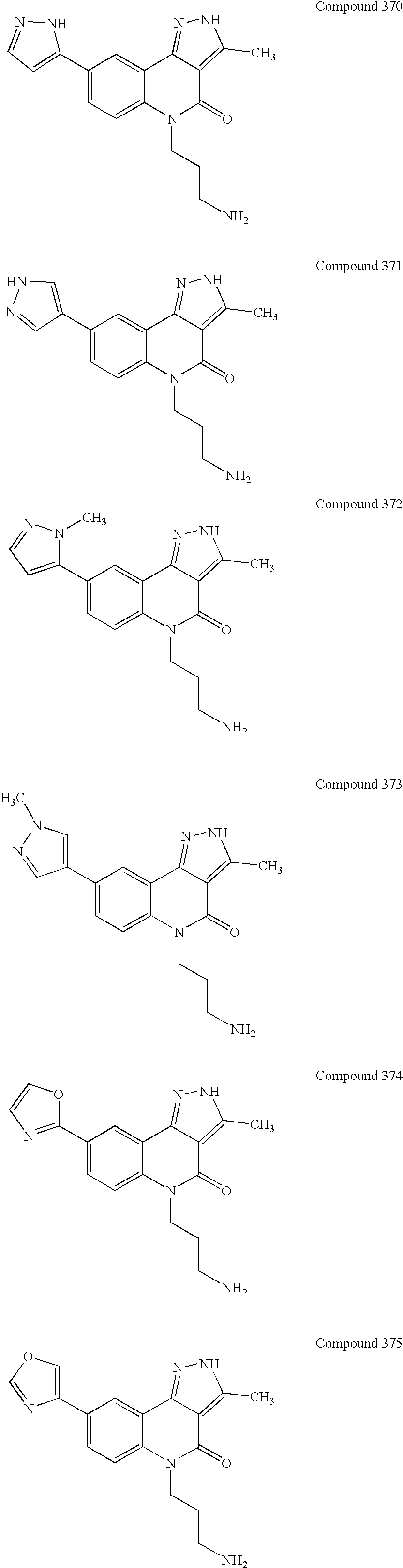

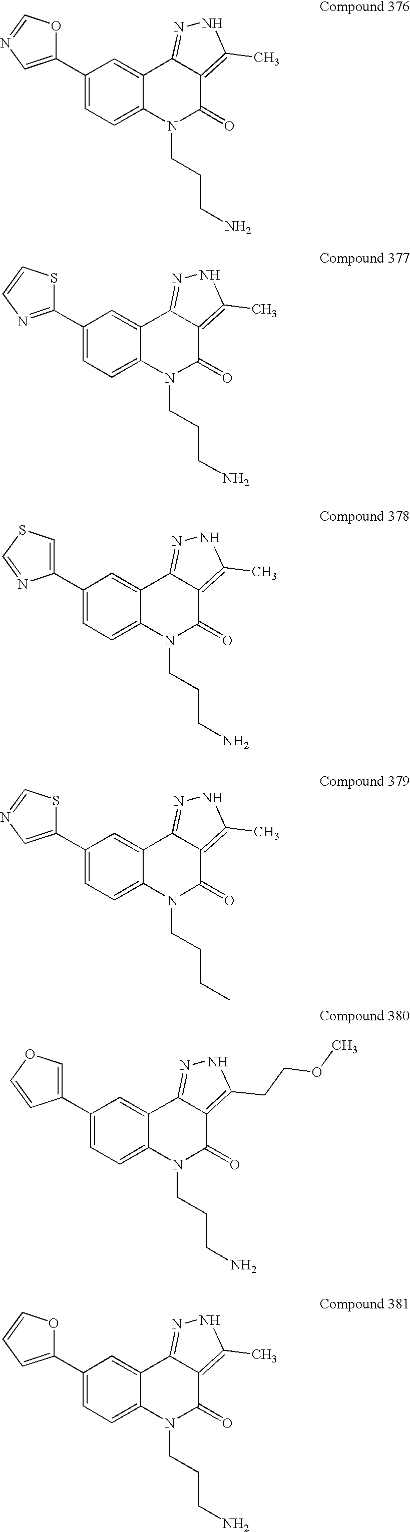

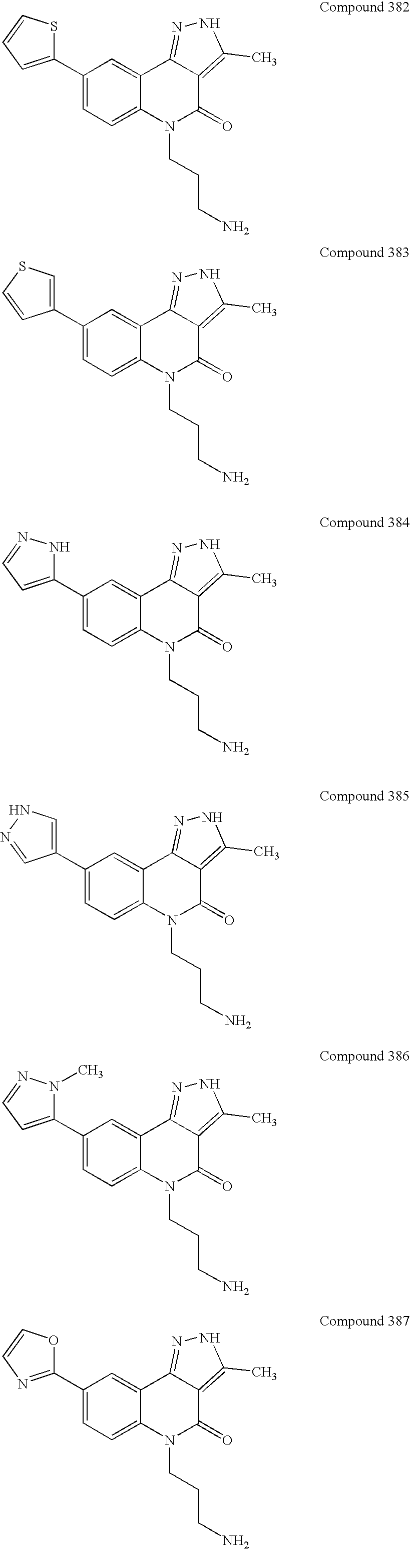

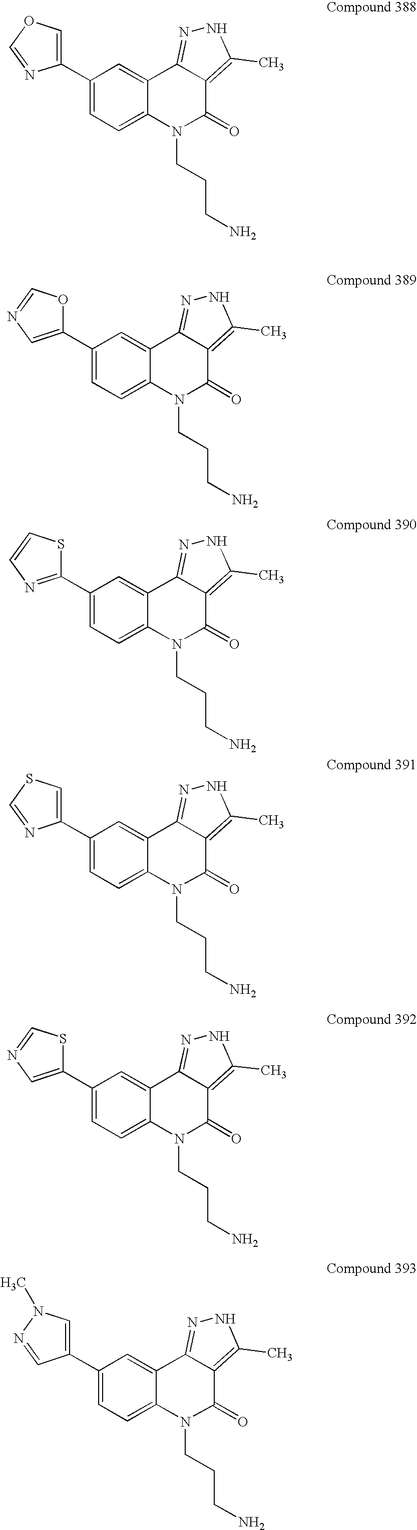

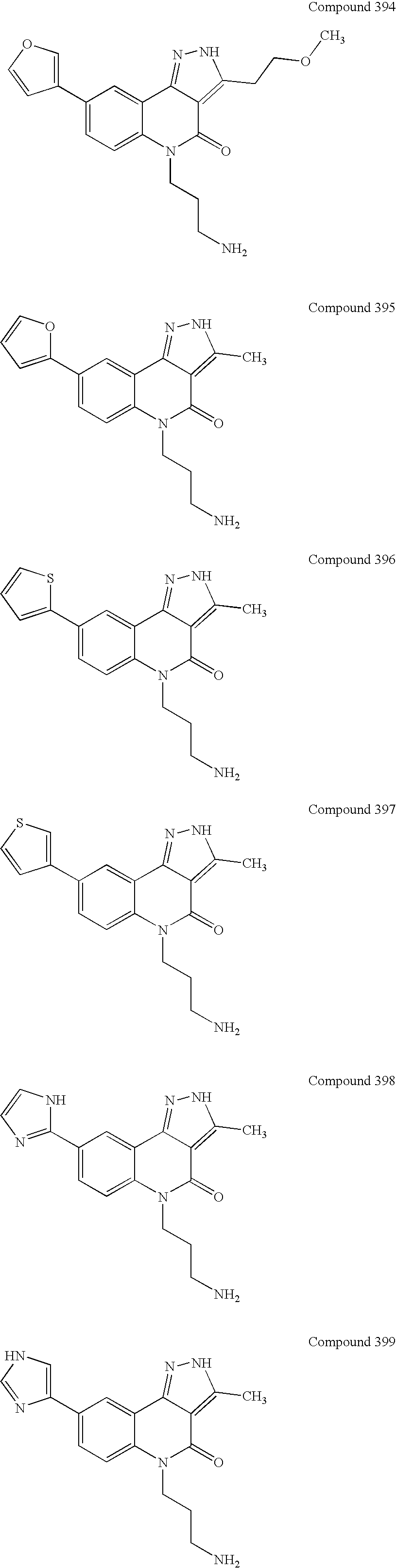

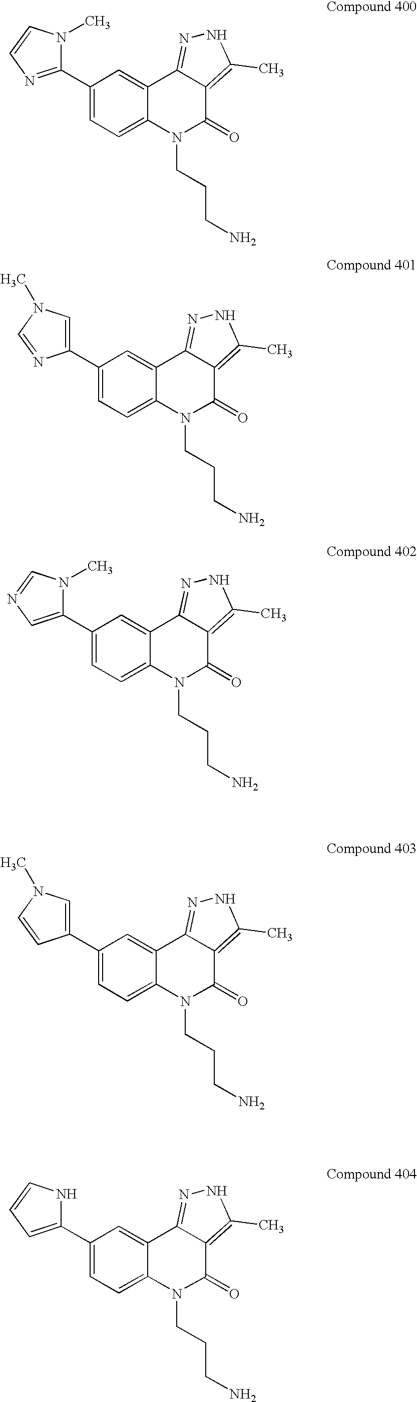

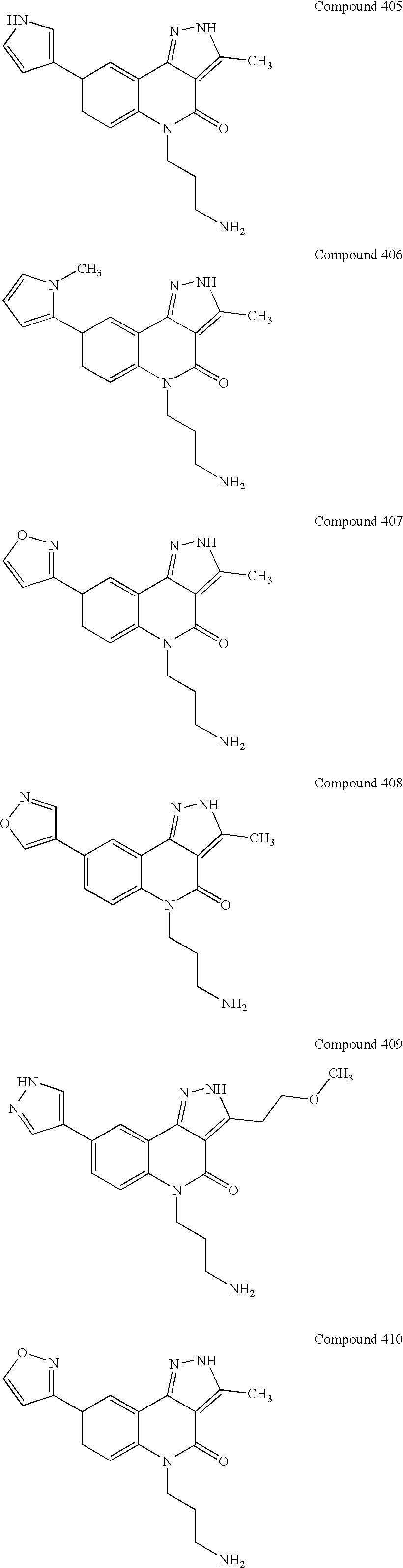

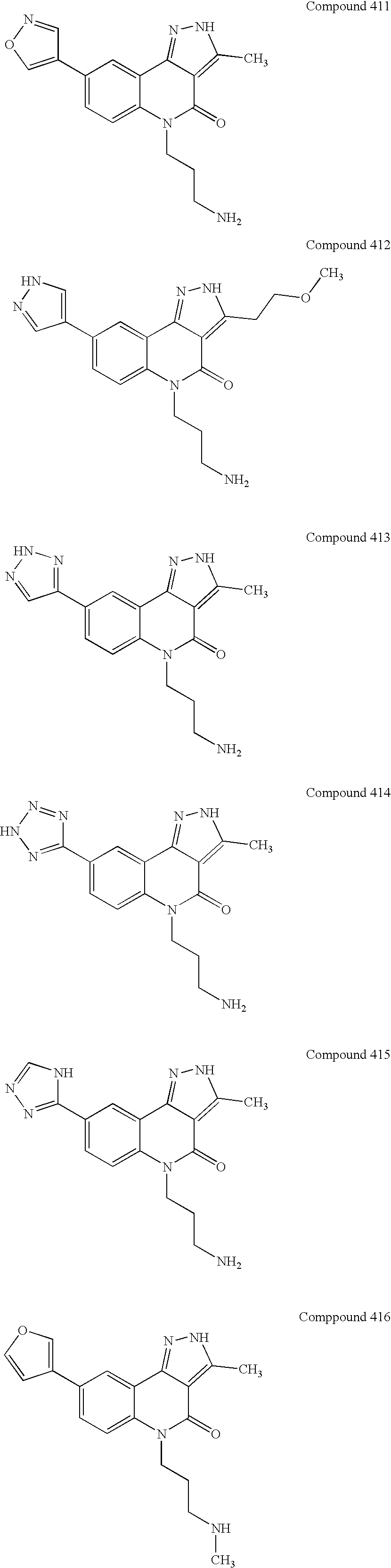

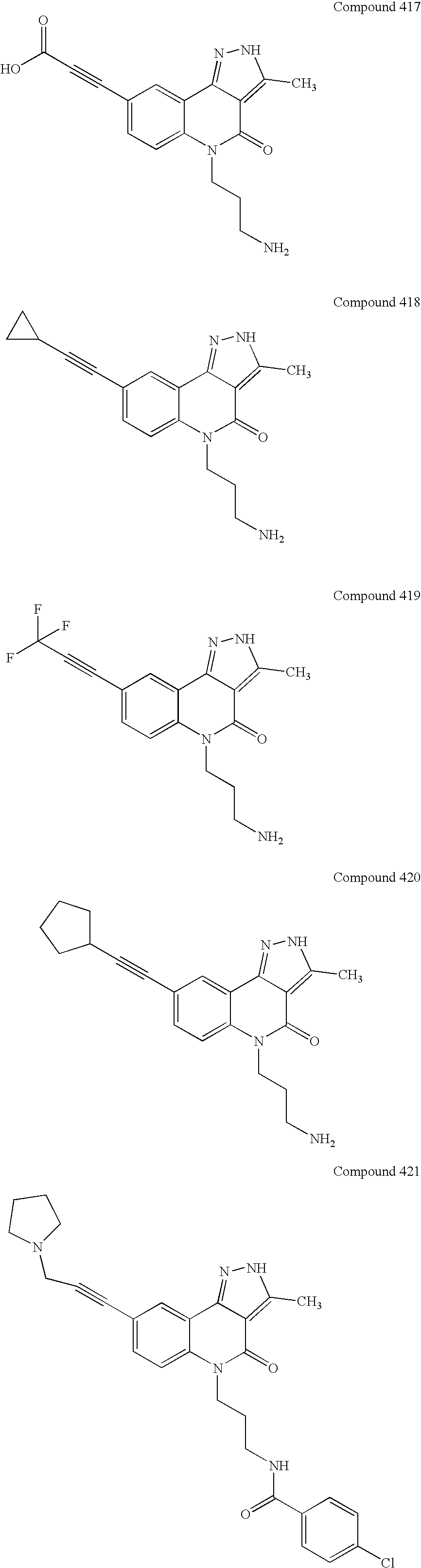

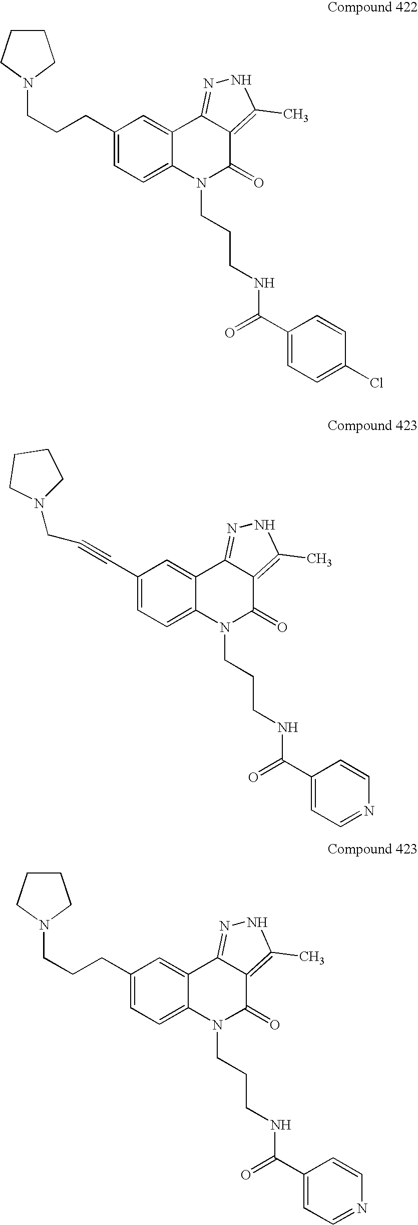

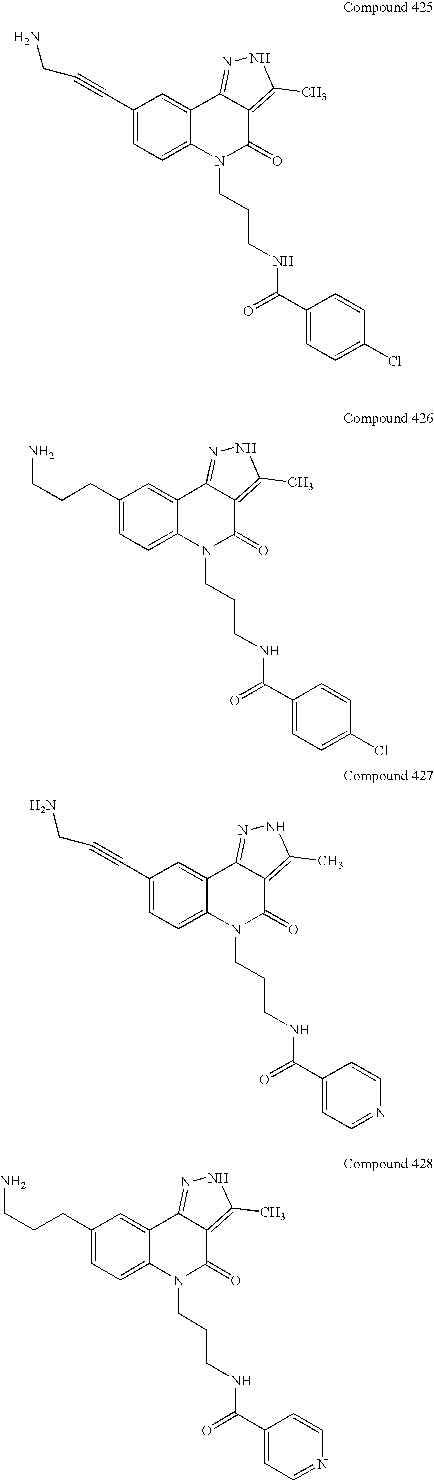

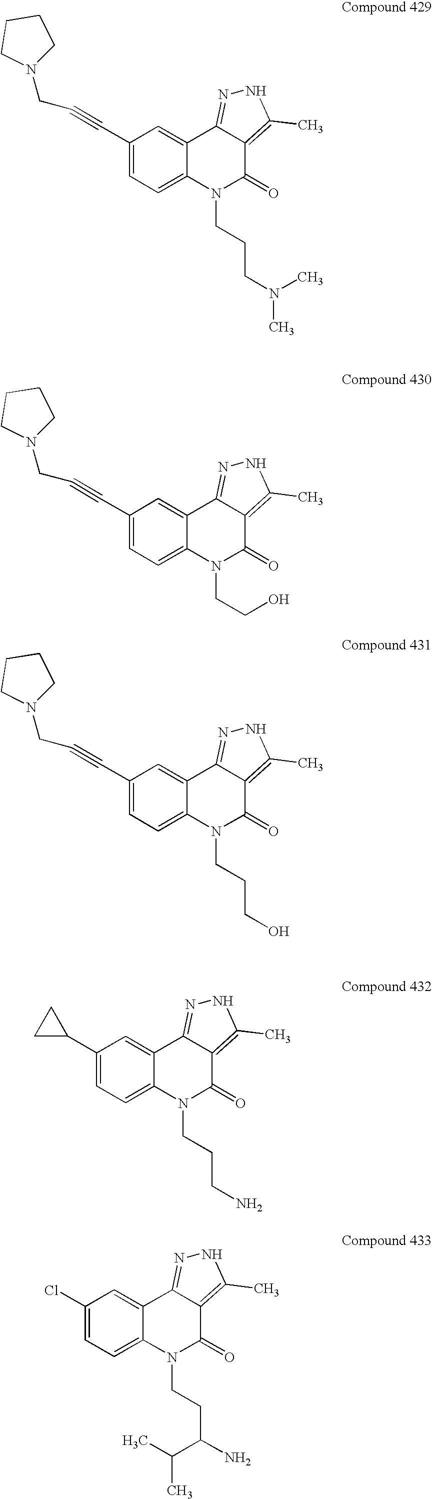

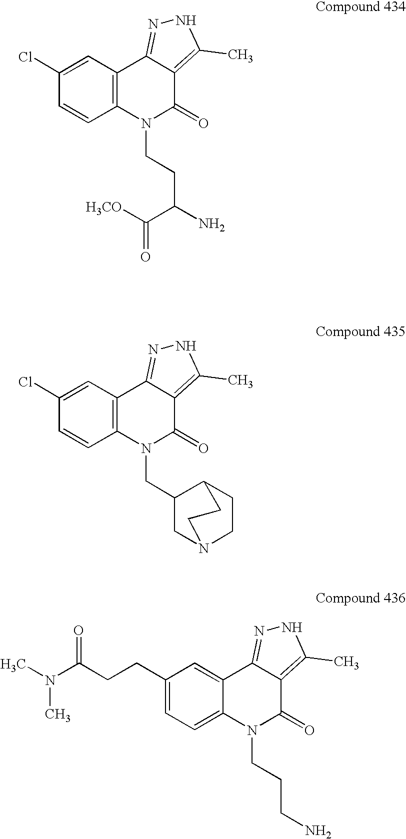

The depiction of R² in Structural Formulas (I)-(V), (VIIa) and (X) indicates that R² is permissibly bonded to either of the nitrogen atoms in the pyrazolo or triazolo ring. Thus, Structural Formula (I) encompasses Structural Formula (XVI and (XVII): 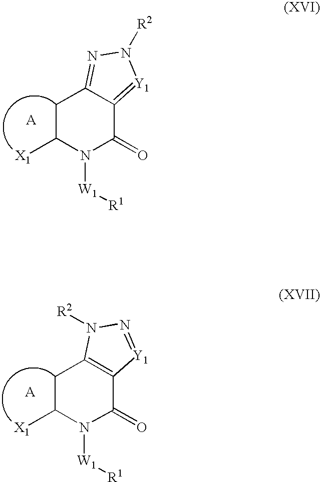
Structural Formulas (III)-(V), (VIIa), (X), (XXXII) and (XXXIII) also encompass R² bonded to either of the nitrogen atoms in the pyrazolo or triazolo ring, as depicted in Structural Formulas (XVI) and (XVII).

R² in Structural Formulas (I)-(V), (VIIa), (X), (XXXII) and (XXXIII) is —H or a group that is cleavable in vivo. The term “cleavable in vivo” means that after the Chk-1 inhibitor is administered to a subject, at least half of the cleavable groups R² groups are converted to —H before half of the administered Chk-1 inhibitor is cleared from the subject or metabolized to a form that is inactive with respect to Chk-1. A cleavable R² group can be converted to —H either by hydrolysis or enzymatically. Examples of suitable cleavable groups for R² include —S(O)₂R to form a sulfonamide, —C(O)—R to form an amide, —C(O)—OR to form a carbamate and —C(O)—NHR or —C(O)—NR₂ to form a urea, wherein R is an optionally substituted alkyl or an optionally substituted aryl group, (preferably an unsubstituted alkyl or an optionally substituted aryl group such as an optionally substituted phenyl group) or —NR² is a substituted or unsubstituted heteroaryl or non-aromatic heterocyclic group. Specific examples of pyrazoles with cleavable groups are shown below:

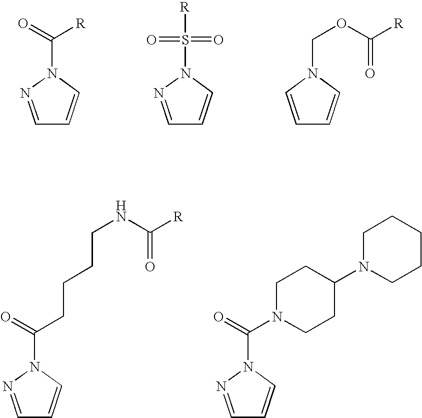

When R² represents —H, two tautomeric forms of the molecule are possible. By way of example, these two tautomeric forms are shown below for Structural Formula (I): 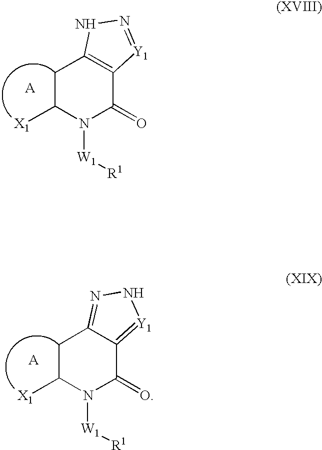
It is to be understood that when the Chk-1 inhibitors disclosed herein are depicted with a structural formula, both tautomeric forms are contemplated.

Some of the disclosed Chk-1 inhibitors contain one or more chiral centers. The presence of chiral centers in a molecule gives rise to stereoisomers. For example, a pair of optical isomers, referred to as “enantiomers”, exist for every chiral center in a molecule; and a pair of diastereomers exist for every chiral center in a compound having two or more chiral centers.

When a disclosed Chk-1 inhibitor is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer. When a mixture is enriched in one enantiomer relative to its optical isomers, the mixture contains, for example, an enantiomeric excess of at least 50%, 75%, 90%, 95% 99% or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed Chk-1 is named or depicted by structure without indicating the stereochemistry and has at least two chiral centers, it is to be understood that the name or structure encompasses a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s). When a mixture is enriched in one diastereomer or diastereomeric pair(s) relative to the other diastereomers or diastereomeric pair(s), the mixture is enriched with the depicted or referenced diastereomer or diastereomeric pair(s) relative to other diastereomers or diastereomeric pair(s) for the compound, for example, by a molar excess of at least 50%, 75%, 90%, 95% 99% or 99.5%.

The diastereoisomeric pairs may be separated by methods known to those skilled in the art, for example chromatography or crystallization and the individual enantiomers within each pair may be separated as described above. In certain instances compounds of the present invention may associated in isolated form with solvent or water, as in a “solvate” or “hydrate”. References to the disclosed compounds or structural formulas depicting the disclosed compounds are meant to include such solvates and hydrates.

The term “alkyl” as used herein means saturated straight-chain, branched or cyclic hydrocarbons. When straight chained or branched, an alkyl group is typically C₁₋₈, more typically C₁₋₆; when cyclic, an alkyl group is typically C₃₋₁₀, more typically C₃₋₇. The terms “alkyl”, “alkoxy”, “hydroxyalkyl”, “alkoxyalkyl”, “alkylamine”, “dialkyamine”, “alkoxycarbonyl” and the like, used alone or as part of a larger moiety includes both straight and branched saturated chains containing one to eight carbon atoms. The term “cycloalkyl” used alone or as part of a larger moiety shall include cyclic C₃-C₁₀ hydrocarbons which are completely saturated

The terms “haloalkyl” and “haloalkoxy” means alkyl or alkoxy, as the case may be, substituted with one or more halogen atoms. The term “halogen” means F, Cl, Br or I.

The term “acyl group” mean —C(O)R, wherein R is an optionally substituted alkyl group or aryl group (e.g., optionally substituted phenyl). R is preferably an unsubstituted alkyl group or phenyl.

An “alkylene group” is represented by —[CH₂]_{z}—, wherein z is a positive integer, preferably from one to eight, more preferably from one to six.

An “alkylidene group” is an alkylene group in which one or more hydrogen atoms are optionally replaced with suitable substituents. Suitable substituents are as defined below for alkyl groups. Preferred substituents include alkyl, hydroxyl, alkoxy, amine, alkylamine, dialkylamine, spiro cycloalkyl, fused cycloalkyl and non-aromatic heterocyclic group. Additional preferred substituents include oxo, halo, hydroxyalkyl, alkoxyalkyl, aminoalkyl. W₁-W₃ are defined to be an alkylidene optionally substituted with inter alia hydroxy, alkoxy and amines. One of ordinary skill in the art will recognize that substitution of the alpha carbon atom of W₁ (the carbon atom bonded to R¹) and the alpha carbon of W₂ and W₃ (the carbon atom which is bonded to R⁵) with a hydroxyl, cyano or amine will result in a functional group which is not sufficiently stable for pharmaceutical use when certain values of R₁ and R⁵ are selected. By way of example, when R₁ or R⁵ is —OH or —CN, substitution of the alpha carbon of W₁-W₃ with —OH will result in —CH(OH)OH and —CH(OH)CN, respectively, both of which are not sufficiently stable for pharmaceutical use. Such groups are not within the scope of the present invention. Thus, when R₁ or R⁵ is —OR¹², —NR¹¹R¹², —CN, —NR¹¹CONR¹¹R¹², —NR¹¹SO₂R¹², —NR¹¹COR¹², —NH—C(═NR¹¹)NR¹¹R¹², —NR¹¹SO₂R¹², —OC(O)R¹², —NR¹¹C(O)OR¹², —OC(O)—NR₁₁R¹², —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹² or —OC(O)—C(R^{12c}R^{12c})—NR¹¹R¹², then the alpha carbon of W₁-W₃ is preferably unsubstituted or optionally substituted with one or two methyl groups or a spiro cycloalkyl group.

W₂ is defined to be a C1-C6 alkylidene group in which one carbon atom in the alkylidene group is optionally replaced with T. Thus, —W₂—R⁵ includes -T-[CH₂]₅—R⁵, —CH₂-T-[CH₂]₄—R⁵, —[CH₂]₂-T-[CH₂]₃—R⁵, —[CH₂]₃-T-[CH₂]₂—R⁵, -T-[CH₂]₄—R⁵, —CH₂-T-[CH₂]₃—R⁵, —[CH₂]₂-T-[CH₂]₂—R⁵, -T-[CH₂]₃—R⁵, —CH₂-T-[CH₂]₂—R⁵ and -T-[CH₂]₂—R⁵ provided, of course, that one or more hydrogen atoms can be replaced with a suitable substituent, as described above.

In addition, —W₂—R⁵ includes —[CH₂]₄-T-[CH₂]—R⁵, —[CH₂]₃-T-[CH₂]—R⁵, —[CH₂]₂-T-[CH₂]—R⁵ and —CH₂-T-CH₂—R⁵. One of ordinary skill in the art will recognize that when —W₂—R⁵ has these values, certain selections of T and R⁵ will result in functional groups which are not sufficiently stable for pharmaceutical use. By way of example, when T is —O— and R⁵ is —H or —CN, —W₂—R⁵ will comprises a —CH₂OCH₂OH or —CH₂OCH₂CN functional group, which are not sufficiently stable for pharmaceutical use. Such selections of T and R⁵ are not within the scope of the present invention. Thus, when —W₂—R⁵ has one of these values and T is —O—, —S—, —N(R⁶)—, —C(O)O—, —C(O)N(R⁶)— or —SO₂N(R⁶)—, then R⁵ is preferably an optionally substituted heteroaryl group, an optionally substituted non-aromatic heterocyclic group, —SO₂NR¹¹R¹², —CONR¹¹R¹², —COOR¹², —CH(NR¹¹R¹²)-Ph, —CH(NR¹¹R¹²)-(cycloalkyl), a cycloalkyl group or a phenyl group substituted with —V₂—OR¹², —V—NR¹¹R¹². Of course, when the alkylidene is described by these values for W₂, T and R⁵, one or more hydrogen atoms in the alkylidene can be replaced with a suitable substituent, as described above.

In addition, —W₂—R⁵ includes —[CH₂]₅-T-R⁵, —[CH₂]₄-T-R⁵, —[CH₂]₃-T-R⁵ and —[CH₂]₂-T-R⁵. One of ordinary skill in the art will recognize what when —W₂—R⁵ has these values, certain selections of T and R⁵ will result in groupings that are not sufficiently stable for pharmaceutical use. By way of example, when T is —O— and R⁵ is —OH or —CN, —W₂—R⁵ will comprise —CH₂₀₀H or —CH₂OCN, which are not sufficiently stable for pharmaceutical use. Such selections of T and R⁵ are not within the scope of the present invention. Thus, when —W₂—R⁵ has these values, R⁵ is preferably an optionally substituted heteroaryl group, an optionally substituted non-aromatic heterocyclic group, a cycloalkyl group or a phenyl group substituted with —V₂—OR¹². Of course, when the alkylidene is described by these values for W₂, T and R⁵, one or more hydrogen atoms in the alkylidene can be replaced with a suitable substituent, as described above.

The term “oxo” means a group of the formula: “═O”.

An “aliphatic group” is non-aromatic, consists solely of carbon and hydrogen and may optionally contain one or more units of unsaturation, e.g., double and/or triple bonds. An aliphatic group may be straight chained, branched or cyclic. When straight chained or branched, an aliphatic group typically contains between about 1 and about 10 carbon atoms, typically between about 1 and about 6 carbon atoms, more typically between about 1 and about 4 carbon atoms. When cyclic, an aliphatic group typically contains between about 3 and about 10 carbon atoms, more typically between about 3 and about 7 carbon atoms. An aliphatic group may be optionally substituted at any “substitutable carbon atom”. A “substitutable carbon atom” in an aliphatic group is a carbon in an aliphatic group that is bonded to one or more hydrogen atoms. One or more hydrogen atoms can be optionally replaced with a suitable substituent group. A “haloaliphatic group” is an aliphatic group, as defined above, substituted with one or more halogen atoms. Suitable substituents on a substitutable carbon atom of an aliphatic group are the same as those for an alkyl group.

A “spiro cycloalkyl” or “spiro non-aromatic heterocyclic” group is a cycloalkyl or non-aromatic heterocyclic group which shares one ring carbon atom with a carbon atom in an alkylene group or alkyl group.

The symbol “>” when used, for example, in a substituent such as >CH₂, means that the carbon atom at the “point” of the “>” symbol is bonded to two adjacent atoms in the molecule to form a cycopropane. Therefore, the prior recitation of —(CH₂)ᵣ—CH(R²²)CH(R²²)—CH₂— as one value for W₃ and the language that both R²²s, taken together, are >CH₂, >CHCH₃ or >C(CH₃)₂ indicate that the following cyclopropane structures are intended:

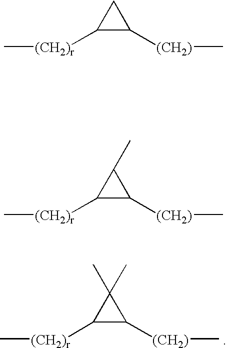

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heteroaryl or non-aromatic heterocyclic group. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR″ (as in N-substituted pyrrolidinyl), wherein R″ is a suitable substituent for the nitrogen atom in the ring of a non-aromatic nitrogen-containing heterocyclic group, as defined below.

The term “aromatic group” used alone or as part of a larger moiety as in “aralkyl”, “aralkoxy”, or “aryloxyalkyl”, includes carbocyclic aromatic rings and heteroaryl rings. The term “aromatic group” may be used interchangeably with the terms “aryl”, “aryl ring” “aromatic ring”, “aryl group” and “aromatic group”.

Carbocyclic aromatic ring groups have only carbon ring atoms and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring”, as it is used herein, is a group in which an aromatic ring is fused to one or more non-aromatic rings (cycloalkyl or heterocyclic), such as in an indanyl, phthalimidyl, naphthimidyl, phenantriidinyl, or tetrahydronaphthyl, where the radical or point of attachment is on the aromatic ring.

The term “heteroaryl”, “heteroaromatic”, “heteroaryl ring”, “heteroaryl group” and “heteroaromatic group”, used alone or as part of a larger moiety as in “heteroaralkyl” or “heteroarylalkoxy”, refers to heteroaromatic ring groups having five to fourteen members, including monocyclic heteroaromatic rings and polycyclic aromatic rings in which a monocyclic aromatic ring is fused to one or more other carbocyclic or heteroaromatic aromatic rings. Examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl. Also included within the scope of the term “heteroaryl”, as it is used herein, is a group in which a heteroaryl ring is fused to one or more cycloalkyl or non-aromatic heterocyclic groups where the radical or point of attachment is on the heteroaromatic ring. Examples include tetrahydroquinolinyl, tetrahydroisoquinolinyl, and pyrido [3, 4-d]pyrimidinyl.

The term “non-aromatic heterocyclic group”, used alone or as part of a larger moiety as in “non-aromatic heterocyclylalkyl group”, refers to non-aromatic ring systems typically having five to fourteen members, preferably five to ten, in which one or more ring carbons, preferably one to four, are each replaced by a heteroatom such as N, O, or S. A “nitrogen-containing non-aromatic heterocyclic group” is a non-aromatic heterocyclic group with a nitrogen ring atom. Examples of non-aromatic heterocyclic groups include 3-1H-benzimidazol-2-one, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, N-morpholinyl, 2-morpholinyl, 3-morpholinyl, N-thiomorpholinyl, 2-thiomorpholinyl, 3-thiomorpholinyl, N-pyrrolidinyl, 2-pyrrolidinyl, 3-pyrrolidinyl, N-piperazinyl, 2-piperazinyl, N-piperidinyl, 2-piperidinyl, 3-piperidinyl, 4-piperidinyl, N-pyrrolidinyl, 2-pyrrolidinyl, 3-pyrrolidinyl, 4-thiazolidinyl, diazolonyl, N-substituted diazolonyl, 1-pthalimidinyl, benzoxanyl, benzopyrrolidinyl, benzopiperidinyl, benzoxolanyl, benzothiolanyl, and benzothianyl. Also included within the scope of the term “non-aromatic heterocyclic group”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The designation “N” on N-morpholinyl, N-thiomorpholinyl, N-pyrrolidinyl, N-piperazinyl and N-piperidinyl indicates that the non-aromatic heterocyclic group is attached to the remainder of the molecule at the ring nitrogen atom.

An “aralkyl group”, “heteroaralkyl group” or “non-aromatic heterocyclylalkyl” are an alkyl group substituted with an aryl, heteroaryl or non-aromatic heterocyclic group, respectively.

The term “ring atom” is an atom such as C, N, O or S that is in the ring of an aromatic group, cycloalkyl group or non-aromatic heterocyclic ring.

A “substitutable ring atom” in an aromatic group is a carbon or nitrogen atom in an aromatic group that is bonded to a hydrogen atom. The hydrogen can be optionally replaced with a suitable substituent group. Thus, the term “substitutable ring atom” does not include ring carbon or nitrogen atoms which are shared when two rings are fused. In addition, “substitutable ring atom” does not include ring carbon or nitrogen atoms when the structure depicts that they are already attached to a moiety other than hydrogen. Thus, the carbon atom bonded to R⁴ in Structural Formula (VI) is not a “substitutable ring atom” within the meaning of the term, as it is used herein.

An aryl group (including, but not limited to Ring A, Ring B, Ring C, Ring E, and aryl groups represented by R¹, R^{3a}, R^{a}, R^{b}, R^{c}, R⁵, R¹², R¹³ and Ph) may contain one or more substitutable ring atoms, each bonded to a suitable substituent. Examples of suitable substituents on a substitutable ring carbon atom of an aryl group include halogen, R^{o}, —OR^{o}, —O(haloalkyl), —SR^{o}, 1,2-methylene-dioxy, 1,2-ethylenedioxy, trialkylsilyl, boronate, alkylboronate, dialkylboronate, —NO₂, —CN, —N(R′)₂, —NR′CO₂R^{o}, —NR′C(O)R^{o}, —NR′NR′C(O)R^{o}, —N(R′)C(O)N(R′)₂, —NR′NR′C(O)N(R′)₂, —NR′NR′CO₂R^{o}, —C(O)C(O)R′, —C(O)CH₂C(O)R^{o}, —CO₂R^{o}, —C(O)R′, —C(O)N(R^{o})₂, —OC(O)R′, —OC(O)N(R^{o})₂, —S(O)₂R^{o}, —SO₂N(R′)₂, —S(O)R^{o}, —NR′SO₂N(R′)₂, —NR′SO₂R^{o}, —C(═S)N(R′)₂, —NR′—C(═NH)—N(R′)₂ or —C(═NH)—N(R′)₂.

Each R′ is independently R^{o}, —CO₂R^{o}, —SO₂R′ or —C(O)R^{o} or —NR′R′ is an optionally substituted non-aromatic nitrogen-containing heterocyclic group;

Each R^{o} is independently hydrogen or an alkyl group, non-aromatic heterocyclic group or aromatic group and the alkyl, non-aromatic heterocyclic group and aromatic group represented by R^{o} is optionally substituted with one or more independently selected groups represented by R^{#}.

R^{#} is R⁺, —OR⁺, —O(haloalkyl), —SR⁺, —NO₂, —CN, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C3 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine; or —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

An alkyl group (including, but not limited to, alkyl groups represented by R¹²) or a non-aromatic heterocyclic group (including, but not limited to, non-aromatic heterocyclic groups represented by R¹, R⁵, R¹², NR^{12a}R^{12a}, R¹³ and —NR¹⁴R¹⁵) may contain one or more substituents. Examples of suitable substituents for an alkyl group or a ring carbon of a non-aromatic heterocyclic group include those listed above for a substitutable carbon of an aryl and the following: ═O, ═S, ═NNHR*, ═NN(R*)₂, ═NNHC(O)R*, ═NNHCO₂(alkyl), ═NNHSO₂(alkyl), ═NR*, spiro cycloalkyl group or fused cycloalkyl group Each R* is independently selected from hydrogen, an unsubstituted alkyl group or a substituted alkyl group. Examples of substituents on the alkyl group represented by R* include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl.

An alkyl or alkylidene group is substituted with a spiro cycloalkyl group when one ring carbon in the cycloalkyl group is also part of the alkyl or alkylidene group. For example, the alkylidene groups corresponding to W₁ in Compounds 130 and 131 below are spiro substituted with cyclopropyl and cyclobutyl group, respectively.

Two rings are fused when they share two adjacent ring atoms. A cycloalkyl group or non-aromatic heterocyclic group is fused to an alkyl or alkylidene group when two adjacent ring carbons from the cycloalkyl group or non-aromatic heterocyclic group are also adjacent carbon atoms in the alkyl or alkylidene group.

A preferred position for substitution of a non-aromatic nitrogen-containing heterocyclic group is the nitrogen ring atom. Suitable substitutents on the nitrogen of a non-aromatic heterocyclic group include —R\hat{ }, —N(R\hat{ })₂, —C(O)R\hat{ }, —CO₂ R\hat{ }, —C(O)C(O)R\hat{ }, —C(O)CH₂C(O)R\hat{ }, —SO₂R\hat{ }, —SO₂N(R\hat{ })₂, —C(═S)N(R\hat{ })₂, —C(═NH)—N(R\hat{ })₂, and —NR\hat{ }SO₂R\hat{ }; wherein R\hat{ } is hydrogen, an alkyl group, a substituted alkyl group, phenyl (Ph), substituted Ph, —O(Ph), substituted —O(Ph), CH₂(Ph), or an unsubstituted heteroaryl or heterocyclic ring. Examples of substituents on the alkyl group or the phenyl ring represented by R\hat{ } include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

Non-aromatic nitrogen containing heterocyclic rings that are substituted on a ring nitrogen and attached to the remainder of the molecule at a ring carbon atom are said to be N-substituted. For example, an N-alkyl-piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′-substituted-N-heterocycles. For example, an N′-acyl-N-pyrazinyl group is attached to the remainder of the molecule at one ring nitrogen atom and substituted at the second ring nitrogen atom with an acyl group.

Additionally, pharmaceutically acceptable salts of the compounds of the disclosed Chk-1 inhibitors (e.g., represented by Formula I and II) are included in the present invention. For example, an acid salt of a compound containing an amine or other basic group can be obtained, by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine.

The disclosed Chk-1 inhibitors are advantageously administered to inhibit Chk-1 in a subject in whom a beneficial therapeutic or prophylactic effect can be achieved by inhibiting Chk-1, i.e., a subject in need of Chk-1 inhibition. A “subject” is a mammal, preferably a human or an animal in need of veterinary treatment, e.g., companion animals (e.g., dogs, cats, and the like), farm animals (e.g., cows, sheep, pigs, horses, and the like), and laboratory animals (e.g., rats, mice, guinea pigs, and the like).

The disclosed Chk-1 inhibitors are particularly useful in therapeutic applications relating to a Chk-1-mediated disorder. As used herein, the term “Chk-1-mediated disorder” includes any disorder, disease or condition which is caused or characterized by an increase in Chk-1 expression or activity, or which requires Chk-1 activity. The term “Chk-1-mediated disorder” also includes any disorder, disease or condition in which inhibition of Chk-1 activity is beneficial.

Chk-1 inhibition can be used to achieve a beneficial therapeutic or prophylactic effect, for example, in subjects with a proliferative disorder. Non-limiting examples of proliferative disorders include chronic inflammatory proliferative disorders, e.g., psoriasis and rheumatoid arthritis; proliferative ocular disorders, e.g., diabetic retinopathy; benign proliferative disorders, e.g., hemangiomas; and cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

Non-limiting examples of solid tumors that can be treated with the disclosed Chk-1 inhibitors include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

Non-limiting examples of hematologic malignancies that can be treated with the disclosed Chk-1 inhibitors include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

The disclosed Chk-1 inhibitors are particularly useful in the treatment of cancers or cell types in which Chk-1 protein or activity is upregulated, including, without limitation, rapidly proliferating cells and drug-resistant cells (Shyjan et al., U.S. Pat. No. 6,723,498 (2004)), as well as retinoblastomas such as Rb negative or inactivated cells (Gottifredi et al., Mol. Cell. Biol., 21:1066 (2001)), or where the ARF^{p14/p19} locus has been inactivated or misregulated. The disclosed Chk-1 inhibitors also are particularly useful in the treatment of cancers or cell types in which another checkpoint pathway has been mutated or abrogated, including, without limitation, cancers or cell types in which p53 or the p53 pathway has been inactivated or abrogated.

The disclosed Chk-1 inhibitors can be administered in conjunction with other therapeutic agents, including anticancer agents. As used herein, the term “anticancer agent” refers to any agent that is administered to a subject with cancer for purposes of treating the cancer. Use of Chk-1 inhibitors for the treatment of cancer is particularly advantageous and can enhance the effectiveness of the treatment when: 1) combined with radiation therapy or chemotherapeutic agents that act by causing damage to the genetic material of cells (collectively referred to herein as “DNA damaging agents”); 2) combined with agents which are otherwise cytotoxic to cancer cells during cell division; 3) combined with agents which are proteasome inhibitors; 4) combined with agents which inhibit NF-κB (e.g., IKK inhibitors) (Bottero et al., Cancer Res., 61:7785 (2001); or 5) used with combinations of cancer drugs with which are not cytotoxic when administered alone, yet in combination produce a toxic effect. In preferred embodiments, a disclosed Chk-1 inhibitor is combined with a DNA damaging agent.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Agents that disrupt cell replication include: paclitaxel, docetaxel, and related analogs; vincristine, vinblastin, and related analogs; thalidomide and related analogs (e.g., CC-5013 and CC-4047); protein tyrosine kinase inhibitors (e.g., imatinib mesylate and gefitinib); antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

The disclosed Chk-1 inhibitors are also effective when used in combination with DNA-damaging anti-cancer drugs and/or radiation therapy to treat subjects with multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, often as many as five or more.

As such, an “effective amount” of the disclosed Chk-1 inhibitors is the quantity which inhibits Chk-1 when administered to a subject or which, when administered to a subject with cancer, slows tumor growth, ameliorates the symptoms of the disease and/or increases longevity. When used in combination with a DNA damaging agent, an effective amount of the Chk-1 inhibitor is the quantity at which a greater response is achieved when the Chk-1 inhibitor is co-administered with the DNA damaging anti-cancer drug and/or radiation therapy than is achieved when the DNA damaging anti-cancer drug and/or radiation therapy is administered alone. When used as a combination therapy, an “effective amount” of the DNA damaging agent is administered to the subject, which is a quantity that normally produces an anti-cancer effect.

A disclosed Chk-1 inhibitor can be co-administered with another therapeutic agent (e.g., DNA-damaging agent, agent that disrupts cell replication, proteasome inhibitor, NF-κB inhibitor, or other anticancer agent) as part of the same pharmaceutical composition or, alternatively, as separate pharmaceutical compositions. When administered separately, the Chk-1 inhibitor can be administered prior to, at the same time as, or following administration of the other agent, provided that the enhancing effect of the Chk-1 inhibitor is retained.

The amount of Chk-1 inhibitor, DNA damaging anti-cancer drug and radiation dose administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person. Effective amounts of the disclosed Chk-1 inhibitors typically range between about 1 mg/mm² per day and about 10 grams/mm² per day, and preferably between 10 mg/mm² per day and about 5 grams/mm².

The Chk-1 inhibitors described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The Chk-1 inhibitor will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

For oral administration, the Chk-1 inhibitor or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch, alginic acid; a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parental administration the disclosed Chk-1 inhibitor, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations described previously, the compounds may also be formulated as a depot preparation. Such long acting formulations may be administered by implantation, for example, subcutaneously or intramuscularly or by intramuscular injection. Thus, for example, as an emulsion in an acceptable oil, or ion exchange resins, or as sparingly soluble derivatives, for example, as sparingly soluble salts.

Preferably disclosed Chk-1 inhibitors or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I, II or III or salts thereof) in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

The disclosed Chk-1 inhibitors can be prepared by a variety of procedures some of which are illustrated in the routes 1-4.

Route 1

The compound of formula XXI may be prepared from the quinoline XXII by reaction with hydrazine. Quinoline XXII can be retraced to the N-alkylated anthranilic acid XXIII. Anthranilic acids represented by XXIII are known in the art, and to the extent not commercially available, are readily synthesised by standard procedures commonly employed in the art. 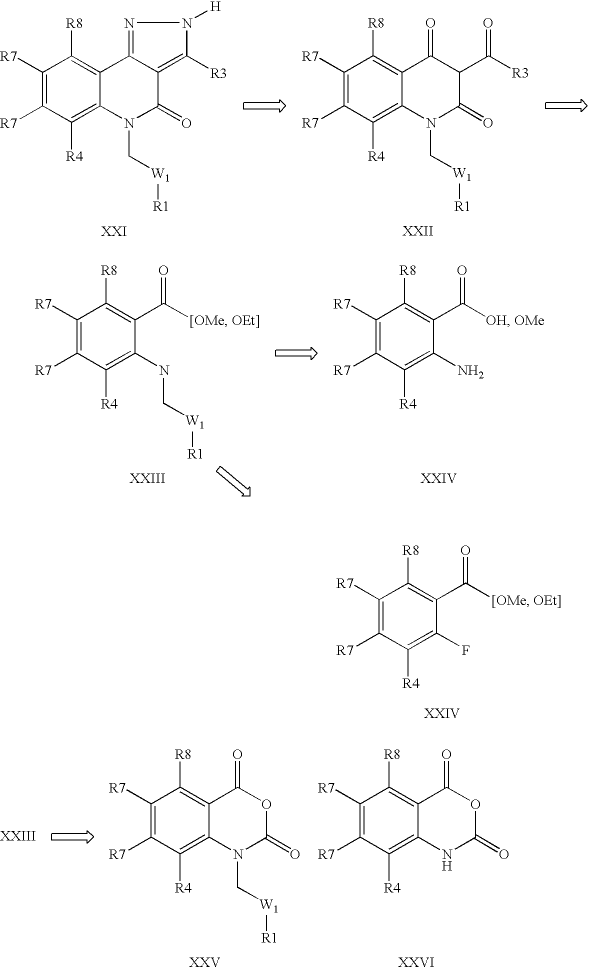
The compound of formula XXIII can also be synthesised from the isatoic anhydride XXV, which can be obtained by alkylation of the parent isatoic anhydride XXVI. Compounds represented by XXVI are commercially available or known in the art. The compound of formula XXIII can also be synthesised by displacement of fluoride of the corresponding 2-fluoro benzoate by a suitable amine.
Route 2

The compound of formula XXI can also be synthesised from XXVIII by deprotection of all protected functional groups at the last stage (exemplified here on the pyrazole). The compound of formula XXVIII can be obtained by alkylation of the suitably protected tricyclic core XXVII with the appropriate halide. 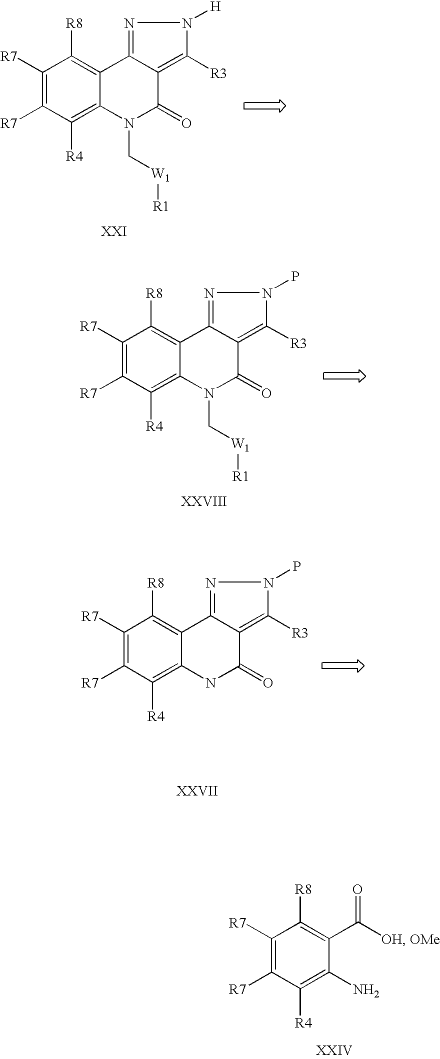
The choice of protecting group will depend on the lability of these compounds and on the side chain introduced. Protecting groups are selected so that they are suitable for the depicted transformations and can be removed following the synthesis with little or no loss of yield. The introduction and selective removal of protecting groups are taught in Greene and Wuts, “Protective Groups in Organic Synthesis”, John Wiley & Sons (1991).
Route 3

The compound of formula XXVIII, which ultimately leads to I, can also be synthesised from XXIX by means of an intramolecular cyclisation (described here by a means of a palladium catalyst) known as a Heck reaction. Other cyclisation conditions can be used if compatible with the protecting groups and functionalities present in XXIX.

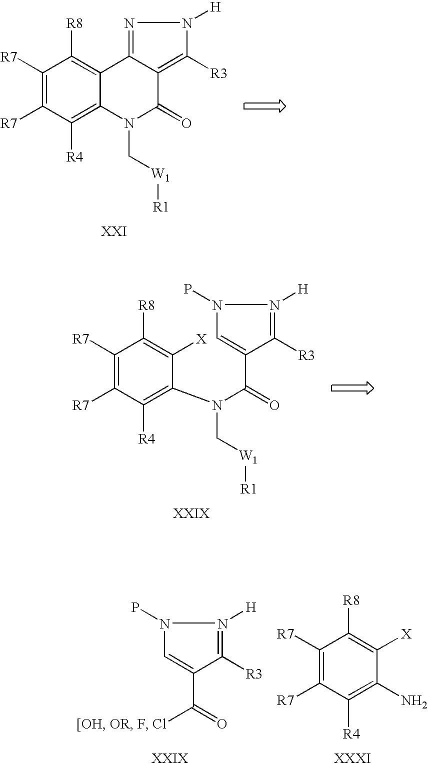

The intermediate XXIX can be traced to the 2-halogeno aryl amine XXXI and the pyrazole (acid, ester, Fluoride, chloride) XXX. 2-Halogeno aryl amines represented by XXXI are known in the art; syntheses for the pyrazole represented by XXX are known in the art and many others are commercially available. 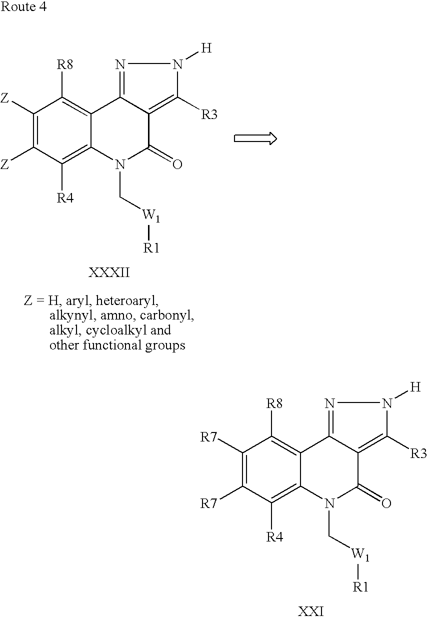
Compounds XXXII, where Z is a variety of functionalities, can be obtained from XXI or a protected version of XXI, where R⁷ is bromo or iodo, by a transition-metal catalyzed coupling reaction or by other methods known in the art.

The invention is illustrated by the following examples which are not intended to be limiting in any way.

### EXEMPLIFICATION

Analytical LC-MS Methods

LCMS (Formic Acid) (Method A)

The compounds were analysed on a Phenomenex Luna column [C18, 50×4.6 mm, 5 um] eluted with 5% acetonitrile/water/0.1% formic acid (mobile phase A) and 100% acetonitrile/0.1% formic acid (mobile phase B) with a flow rate of 1.5 ml/min. The 5 min cycle consisted of a gradient of 100% A to 100% B in 3.5 min; 100% B for 1 min; 100% B to 100% A in 0.1 min; then re-equilibration with mobile phase A for 0.49 min.

LCMS (Ammonium Acetate/Ammonium Formate) (Method B)

Analysed by the same procedure as described above for formic acid but with the mobile phases 5% methanol/water/5 mM ammonium acetate or ammonium formate (A) and 100% methanol/5 mM ammonium acetate or ammonium formate (B).

LCMS (Formic Acid), Long Run, (Method C)

The compounds were analysed on a Phenomenex Luna column [C18, 150×4.6 mm, 5 um] eluted with acetonitrile (generally either 5%, 20% or 40%)/water/0.1% formic acid (mobile phase A) and 100% acetonitrile/0.1% formic acid (mobile phase B) and a flow rate of 1.0 ml/min. The 16 min cycle included a 10 min gradient of 100% A to 100% B; 100% B for 2 min; then re-equilibration to 100% A.

LCMS (Ammonium Acetate). Long Run. (Method D)

Analysed by the same 16 min cycle as above for formic acid but with the mobile phases methanol (generally either 5%, 20% or 40%)/water/5 mM ammonium acetate (A) and 100% methanol/5 mM ammonium acetate (B).

LCMS conditions: spectra were run on a Phenominex Luna 5u C18 50×4.6 mm column on a Hewlett-Packard HP 1100 at 2.5 ml/min for a 3 minute run using the following gradients: Method Polar Formic Acid (PFA): Acetonitrile containing zero to 50 percent 0.1% formic acid in water.

Method Formic Acid (FA): Acetonitrile containing zero to 100 percent 0.1% formic acid in water.

Method Nonpolar Formic Acid (NFA) Acetonitrile containing 70 to 100 percent 0.1% formic acid in water.

Method Polar Ammonium Acetate (PAA): Acetonitrile containing zero to 50 percent 10 mM ammonium acetate in water.

Method Ammonium Acetate (AA): Acetonitrile containing zero to 100 percent 10 mM ammonium acetate in water.

Method Nonpolar Ammonium Acetate (NAA). Acetonitrile containing 70 to 100 percent 10 mM ammonium acetate in water.

### EXAMPLE 1

### Preparation of [2-(3-Methyl-4-oxo-1,4-dihydro-pyrazolo[4,3-c]quinolin-5-yl)-ethyl]-carbamic acid tert-butyl ester (route 1)

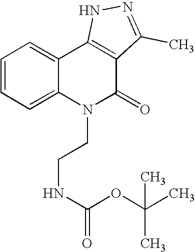

### Step 1: Preparation of 2-(2-tert-Butoxycarbonylamino-ethylamino)-benzoic acid methyl ester

A solution of methyl anthranilate (0.815 mL, 6.3 mmol, 1 equiv.) and tert-butyl N-(2-oxoethyl)carbamate (1.00 g, 6.3 mmol, 1 equiv.) in DCM (20 mL) is treated with acetic acid (0.540 mL, 9.5 mmol, 1.5 equiv.) and stirred for 1 h before portionwise addition of sodium triacetoxyborohydride (2.14 g, 10.1 mmol, 1.6 equiv.) and stirring for a further 18 h. Methanol (20 mL) is added and the quenched solution concentrated in vacuo. The residue is partitioned between ethyl acetate and a saturated aqueous solution of NaHCO₃. The separated aqueous layer is extracted with ethyl acetate and the combined organics washed with sat. NaHCO_{3 (aq)} then brine, dried over Na₂SO₄, filtered and concentrated. The crude material is purified by silica gel chromatography, eluting with 10% EtOAc/hexane. The desired product is obtained as a clear, colourless oil (775 mg, 42% yield). ¹H NMR (400 MHz, CDCl₃) δ 8.14 (1H, dd, J=8.0, 1.5 Hz); 7.60 (1H, m); 7.06 (1H, d, J=8.2 Hz); 6.89 (1H, t, J=7.5 Hz); 5.07 (1H, br s); 4.09 (3H, s); 3.62 (4H, m); 1.68 (9H, s).

### Step 2: Preparation of [2-(3-Acetyl-4-hydroxy-2-oxo-2H-quinolin-1-yl)-ethyl]-carbamic acid tert-butyl ester

A solution of 2-(2-tert-butoxycarbonylamino-ethylamino)-benzoic acid methyl ester (0.700 g, 2.37 mmol, 1 equiv.) and 2,2,6-trimethyl-1,3-dioxin-4-one (0.345 mL, 2.62 mmol, 1.1 equiv.) in toluene (10 mL) is divided into three and each portion microwave irradiated at 140° C. for 600 s. The product solutions are combined and the solvent removed in vacuo. The residue is purified by silica gel chromatography (1:1 EtOAc/hexane). The purified product (0.65 g, 72% yield) is dissolved in ethanol (15 mL), treated with sodium ethoxide (0.476 g, 7 mmol, 4 equiv.) and the solution heated at reflux for 2 h. After cooling the solution is quenched with 1M HCl_{(aq)} (7 mL) and the solvent removed in vacuo. The solid residue is taken up in water and filtered, then washed with water twice and diethyl ether twice, providing a light orange solid (496 mg, 84% yield). ¹H NMR (400 MHz, DMSO-d₆) δ=8.22 (1H, d, J=7.8 Hz); 7.90 (1H, t, J=7.7 Hz); 7.76 (1H, d, J=8.7 Hz); 7.42 (1H, t, J=7.5 Hz); 7.08 (1H, t, J=5.6 Hz); 4.33 (2H, t, J=6.5 Hz); 3.30 (2H, m); 2.83 (3H, s); 1.41 (9H, s).

### Step 3: Preparation of [2-(3-Methyl-4-oxo-2,4-dihydro-pyrazolo[4,3-c]quinolin-5-yl)-ethyl]-carbamic acid tert-butyl ester

A slurry of [2-(3-acetyl-4-hydroxy-2-oxo-2H-quinolin-1-yl)-ethyl]-carbamic acid tert-butyl ester (0.173 g, 0.5 mmol) an DMF (2 mL) is treated with hydrazine hydrate (0.090 mL, 1.5 mmol, 3 equiv.) and the resultant solution microwave irradiated at 200° C. for 300 s. The solvent is removed in vacuo and the white solid residue taken up in water, filtered, washed with water and dried. The desired product is obtained as an off-white solid (0.120 g, 70% yield). ¹H NMR (400 MHz, DMSO-d₆) δ 8.10 (1H, d, J=7.6 Hz); 7.70 (1H, m); 7.58 (1H, t, J=7.6 Hz); 7.30 (1H, t, J=7.4 Hz); 7.03 (1H, t, J=5.4 Hz); 4.27 (2H, t, J=7.4 Hz); 3.20 (2H, q, J=6.3 Hz); 2.57 (3H, s); 1.35 (9H, s).

LCMS: Method B, Rₜ=3.38 min, [MH⁺]=343.

### EXAMPLE 2

### Preparation of 3-Methyl-5-pyridin-3-ylmethyl-1,5-dihydro-pyrazolo[4,3-c]quinolin-4-one

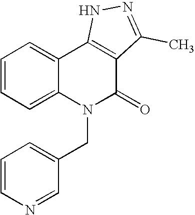

This compound was prepared from the appropriate reagents by an analogous procedure to Example 1. ¹H NMR (400 MHz, CDCl₃); δ 8.57 (1H, d, J=2.0 Hz); 8.46 (1H, dd, J=4.8, 1.2 Hz); 8.02 (1H, m); 7.59 (1H, d, J=8.0 Hz); 7.36 (1H, m); 7.25 (1H, dd, J=8.0; 4.8 Hz); 7.22-7.17 (1H, m); 7.14 (1H, d, J=8.4 Hz); 5.54 (2H, s); 2.72 (3H, s). LCMS: Method B, Rₜ=3.10 min, M+H⁺=291.

### EXAMPLE 3

### Preparation of Acetic acid 2-(3-methyl-4-oxo-1,4-dihydro-pyrazolo[4,3-c]quinolin-5-yl)-ethyl ester

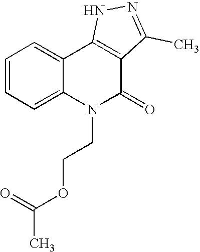

### Step 1: Preparation of 2-(2-Hydroxy-ethylam.no)-benzoic acid methyl ester

A solution of methyl anthranilate (2.6 mL, 20 mmol, 1 equiv.) in DCM (60 mL) is treated with glycolaldehyde dimer (1.20 g, 10 mmol, 0.5 equiv.) then acetic acid (1.72 mL, 30 mmol, 1.5 equiv.) Within 1 h a yellow solution had formed, to which was added portionwise sodium triacetoxyborohydride (6.78 g, 32 mmol, 1.6 equiv.). After 3 days the reaction is quenched with methanol (25 mL) and the solvent removed in vacuo. The residue is partitioned between ethyl acetate and 10% aqueous citric acid. The separated aqueous layer is extracted with ethyl acetate three times and the combined organics washed with brine, dried over Na₂SO₄, filtered and concentrated. The crude product is purified by silica gel chromatography (30% EtOAc/hexane gradient) to provide the desired product as a white waxy solid (1.82 g, 47% yield). ¹H NMR (400 MHz, CDCl₃); δ 7.84 (1H, dd, J=8.0, 1.6 Hz); 7.29 (1H, m); 6.68 (1H, d, J=8.4 Hz); 6.55 (1H, m); 3.81 (2H, t, J=5.5 Hz); 3.78 (3H, s); 3.34 (2H, t, J=5.5 Hz).

### Step 2: Preparation of 2-(2-Acetoxy-ethylamino)-benzoic acid methyl ester

A solution of 2-(2-hydroxy-ethylamino)-benzoic acid methyl ester (1.10 g, 5.6 mmol, 1 equiv.) in DCM (15 mL) is treated successively with triethylamine (0.935 mL, 6.7 mmol, 1.2 equiv.), acetic anhydride (0.585 mL, 6.2 mmol, 1.1 equiv.) and 4-(dimethylamino)pyridine (50 mg, 0.41 mmol) and the solution stirred over night. The orange solution is partitioned between water and ethyl acetate, and the aqueous layer separated and extracted with ethyl acetate twice. The combined organics are washed with water, brine, dried over Na₂SO₄, filtered and concentrated. The crude orange oil is purified by silica gel chromatography (25% EtOAc/hexane) providing the desired acetate as a clear, colourless oil (1.14 g, 85% yield). ¹H NMR (400 MHz, CDCl₃); δ 7.81 (1H, dd, J=8.0, 1.6 Hz); 7.27 (1H, m); 6.63 (2H, d, J=8.4 Hz); 6.53 (1H, m); 4.20 (2H, t, J=5.8 Hz); 3.76 (3H, s); 3.40 (2H, t, J=5.8 Hz); 1.99 (3H, s).

### Step 3: Preparation of Acetic acid 2-(3-acetyl-4-hydroxy-2-oxo-2H-quinolin-1-yl)-ethyl ester

A solution of 2-(2-acetoxy-ethylamino)-benzoic acid methyl ester (1.10 g, 4.6 mmol, 1 equiv.) and 2,2,6-trimethyl-1,3-dioxin-4-one (0.635 mL, 4.8 mmol, 1.05 equiv.) in toluene (5 mL) is treated with DMAP (60 mg, 0.46 mmol, 0.1 equiv.) and heated to reflux. After 16 h the solution is allowed to cool and diluted with sat. NaHCO_{3 (aq)} (100 mL) and ethyl acetate (100 mL). The aqueous phase is extracted twice with ethyl acetate and the combined organics washed with sat. NaHCO_{3 (aq)} and brine, dried over Na₂SO₄, filtered and concentrated. The desired product is separated from unreacted starting material by silica gel chromatography (20%-25%-50% EtOAc/hexane) providing 0.288 g (22% yield) of a yellow solid. 0.84 g (57%) of the starting material is recovered. ¹H NMR (400 MHz, CDCl₃); δ=8.17 (1H, dd, J=8.0, 1.4 Hz); 7.63 (1H, m); 7.39 (1H, m); 7.19 (1H, t, J=8.1 Hz); 4.43 (2H, t, J=6.4 Hz); 4.32 (2H, t, J=6.2 Hz); 2.75 (3H, s); 1.94 (3H, s).

### Step 4: Preparation of Acetic acid 2-(3-methyl-4-oxo-2,4-dihydro-pyrazolo[4,3-c]quinolin-5-yl)-ethyl ester

A solution of acetic acid 2-(3-acetyl-4-hydroxy-2-oxo-2H-quinolin-1-yl)-ethyl ester (0.060 g, 0.21 mmol) in DMF (1 mL) is treated with hydrazine hydrate (0.038 mL, 0.63 mmol, 3 equiv.) and heated at 120° C. for 2 h. After cooling the solvent is removed in vacuo and the residue purified by silica gel chromatography (70% EtOAc/hexane) providing a white solid (0.041 g, 69% yield). ¹H NMR (400 MHz, DMSO-D₆); δ 8.06 (1H, dd, J=8.0, 1.2 Hz); 7.58 (1H, s); 7.52 (1H, t, J=8.0 Hz); 7.25 (1H, t, J=7.4 Hz); 4.46 (2H, t, J=6.2 Hz); 4.24 (2H, t, J=6.0 Hz); 2.52 (3H, s); 1.85 (3H, s). LCMS: Method B, Rₜ=3.12 min, [MNa]=308.

### EXAMPLE 4

### Preparation of 5-(2-Benzylamino-ethyl)-3-methyl-1,5-dihydro-pyrazolo[4,3-c]quinolin-4-one

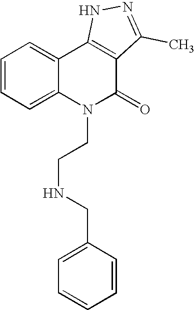 Prepared from Example 11 by the following procedure.

A slurry of 5-(2-amino-ethyl)-3-methyl-1,5-dihydro-pyrazolo[4,3-c]quinolin-4-one (0.070 g, 0.25 mmol, 1 equiv.), benzaldehyde (0.026 mL, 0.25 mmol, 1 equiv.) and diisopropylethylamine (0.044 mL, 0.25 mmol) in THF (1 mL) is treated with sodium triacetoxyborohydride (0.106 g, 0.5 mmol, 2 equiv.) and stirred over night. After quenching with methanol (10 mL) the solution is concentrated and the residue partitioned between ethyl acetate and sat. NaHCO_{3 (aq)}. The organic phase is washed with brine, dried over Na₂SO₄, filtered and concentrated. The crude white solid is purified by silica gel chromatography (EtOAc—95:5 EtOAc/MeOH—92:5:3 EtOAc/MeOH/NEt₃) proving the desired compound as a white solid (0.025 g, 30% yield). ¹H NMR (400 MHz, MeOH-d₄) δ 8.12 (1H, d, J=7.7 Hz); 7.57 (2H, m); 7.4-7.2 (6H, m); 4.50 (2H, t, J=7.3 Hz); 3.85 (3H, s); 2.95 (2H, t, J=7.3 Hz); 2.68 (3H, s). LCMS: Method B, Rₜ=3.31 min. m/z=331 (ES−, M−H), 333 (ES+, M+H). The free amine is converted to the hydrochloride salt in an analogous procedure to Step 5 in Example 7. ¹H NMR (400 MHz, MeOH-D₄); δ 8.06 (1H, d, J=8.0 Hz); 7.54 (1H, m); 7.47 (1H, m); 7.41 (2H, m); 7.34 (3H, m); 7.28 (1H, t, J=7.4 Hz); 4.63 (2H, t, J=5.4 Hz); 4.20 (2H, s); 3.40 (2H, t, J=5.8 Hz); 2.58 (3H, s). LCMS: Method B, RT=3.31 min, [M+H⁺]=333.

### EXAMPLE 5

### Preparation of 5-(3-Benzylamino-propyl)-3-methyl-1,5-dihydro-pyrazolo[4,3-c]quinolin-4-one

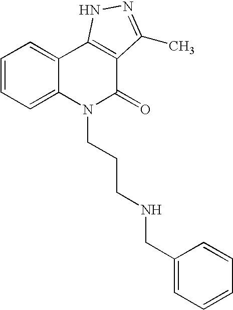

Prepared from Example 15 by an analogous procedure to the conversion of Example 11 to Example 4. ¹H NMR (400 MHz, DMSO-d₆); δ 9.12 (1H, br s); 8.11 (1H, dd, J=8.0, 1.2 Hz); 7.60-7.49 (2H, m); 7.48-7.42 (5H, m); 7.27 (1H, t, J=7.2 Hz); 4.29 (2H, m); 4.06 (2H, m); 2.95 (2H, m); 2.52 (3H, s); 2.02 (2H, quintet, J=7.3 Hz).

LCMS: Method B, Rₜ=2.98 min, M+H⁺=347.

### EXAMPLE 6

### Preparation of [3-(3-Methyl-4-oxo-1,4-dihydro-pyrazolo[4,3-c]quinolin-5-yl)-propyl]-carbamic acid tert-butyl ester

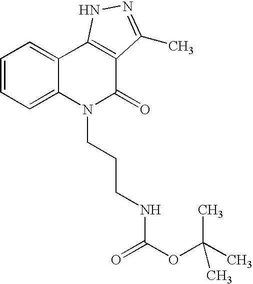

### Step 1: Preparation of Preparation of [3-(2,4-Dioxo-4H-benzo[d][1,3]oxazin-1-yl)-propyl]-carbamic acid tert-butyl ester

A solution of isatoic anhydride (8.16 g, 50 mmol, 1 equiv.) in DMF (80 mL) was treated with K₂CO₃ (7.60 g, 55 mmol, 1.1 equiv.) and stirred for 1 h, after which a solution of 3-bromopropyl carbamic acid tert-butyl ester (12.5 g, 52.5 mmol, 1.05 equiv.) in DMF (20 mL) was added and the mixture stirred at room temperature for 3 days. Water (100 mL) was added and the solution extracted with ethyl acetate (2×200 mL). The combined organics were washed with water 3 times, brine twice, then dried over Na₂SO₄, filtered and concentrated. The crude product was purified by silica gel chromatography using a gradient of 20%-35%-50% ethyl acetate/isohexane to provide 8.54 g (53% yield) of a pale yellow gum. ¹H NMR (CDCl₃, 400 MHz) δ 8.20 (1H, dd, J=7.9, 1.5 Hz); 7.79 (1H, td, J=8.0, 1.7 Hz), 7.34 (1H, t, J=7.6 Hz); 7.22 (1H, d, J=8.5 Hz); 5.09 (1H, m); 4.18 (2H, t, J=7.1 Hz); 3.26 (2H, q, J=6.3 Hz); 1.99 (2H, quintet, J=6.9 Hz); 1.47 (9H, s). In the remaining steps the intermediates are prepared as Example 1, from the appropriate reagents. ¹H NMR (400 MHz, DMSO-d₆); δ 8.12 (1H, d, J=7.6 Hz); 7.55 (2H, m); 7.31 (1H, m); 6.89 (1H, t, J=6.8 Hz); 4.25 (2H, t, J=7.4 Hz); 3.04 (2H, m); 2.58 (3H, s); 1.75 (2H, m); 1.39 (9H, s). LCMS: Method B, Rₜ=3.48 min, [M+H⁺=357].

### EXAMPLE 7

### Preparation of 5-(2-Dimethylamino-ethyl)-3-methyl-1,5-dihydro-pyrazolo[4,3-c]quinolin-4-one hydrochloride

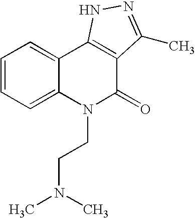

Prepared as follows:

### Step 1 as Example 3

### Step 2: Preparation of 2-(2-Dimethylamino-ethylamino)-benzoic acid methyl ester

A solution of 2-(2-hydroxy-ethylamino)-benzoic acid methyl ester (0.390 g, 2 mmol, 1 equiv.) and triethylamine (0.335 mL, 2.4 mmol, 1:2 equiv.) in DCM (5 mL) is cooled to −40° C. and treated dropwise with methanesulphonyl chloride. After 2 h the suspension is allowed to warm to room temperature and filtered. The residue is washed with DCM and the combined filtrate washed with brine, dried over Na₂SO₄, filtered and concentrated.

The crude mesylate is dissolved in acetonitrile (5 mL) and treated with dimethylamine hydrochloride (0.326 g, 4 mmol, 2 equiv.) and potassium carbonate (1.1 g, 8 mmol, 4 equiv.). The slurry is heated to reflux for 1.5 h before cooling. Water and ethyl acetate are added and the aqueous layer separated and extracted with ethyl acetate.

The combined organics are washed with brine, dried over Na₂SO₄, filtered and concentrated. The crude is purified by silica gel chromatography (EtOAc then 90:5:5 EtOAc/methanol/triethylamine) providing the desired product as a pale yellow oil (0.244 g, 55% yield). ¹H NMR (400 MHz, CDCl₃) δ 7.91 (1H, dd, J=8.0, 1.5 Hz);

7.84 (1H, br s); 7.36 (1H, m); 6.69 (1H, d, J=8.4 Hz); 6.59 (1H, m); 3.86 (3H, s); 3.31 (2H, q, J=7.4 Hz); 2.63 (2H, t, J=6.5 Hz); 2.33 (6H, s).

### Step 3: Preparation of 3-Acetyl-1-(2-dimethylamino-ethyl)-4-hydroxy-1H-quinolin-2-one

A solution of 2-(2-dimethylamino-ethylamino)-benzoic acid methyl ester (0.235 g, 1.06 mmol) and 2,2,6-trimethyl-1,3-dioxin-4-one (0.155 mL, 1.16 mmol, 1.1 equiv.) in toluene is microwave irradiated at 140° C. for 600 s. The solvent is removed in vacuo and the residue purified by silica gel chromatography (95:5 EtOAc/7M NH₃/MeOH) providing 0.116 g (36% yield) of ˜75% pure material. A solution of this material (0.116 g, 0.38 mmol in ethanol (3 mL) is treated with sodium ethoxide (0.102 g, 1.5 mmol) and heated at reflux for 2.5 h. After cooling 1M HCl_{(aq)} (1.5 mL) is added and the solvent removed in vacuo. The residue is taken up in ethyl acetate and filtered. The residual solid (0.164 g) is the hydrochloride salt of the desired product contaminated with sodium chloride. The NMR sample in DMSO is filtered through cotton wool before analysis. ¹H NMR (400 MHz, DMSO-D₆) δ 8.16 (1H, d, J=7.8 Hz); 7.85 (2H, m); 7.39 (1H, m); 4.63 (2H, t, J=7.3 Hz); 3.31 (2H, m); 2.88 (6H, s); 2.75 (3H, s).

### Step 4: Preparation of 5-(2-Dimethylamino-ethyl)-3-methyl-1,5-dihydro-pyrazolo[4,3-c]quinolin-4-one

A slurry of 3-acetyl-1-(2-dimethylamino-ethyl)-4-hydroxy-1H-quinolin-2-one (see above, roughly 0.26 mmol) in DMF (1 mL) is treated with hydrazine hydrate (0.090 mL, 1.5 mmol, 6 equiv.) and the mixture microwave irradiated at 200° C. for 300 s. The cooled mixture is filtered and the solid washed with ethyl acetate four times. The filtrate is concentrated to a pale yellow solid which is purified by silica gel chromatography (93:7 DCM/7M NH₃ in MeOH) providing the desired product as a pale yellow solid (0.06 g, 85% yield). ¹H NMR (400 MHz, DMSO-d₆) δ 8.11 (1H, dd, J=7.7, 1.4 Hz); 7.59 (1H, m); 7.52 (1H, d, J=8.4 Hz); 7.30 (1H, t, J=7.8 Hz), 4.35 (2H, t, J=7.4 Hz); 2.58 (3H, s); 2.47 (2H, t, J=7.3 Hz); 2.25 (6H, s).

### Step 5

A solution of 5-(2-dimethylamino-ethyl)-3-methyl-1,5-dihydro-pyrazolo[4,3-c]quinolin-4-one (0.048 g, 0.18 mmol) in methanol (2 mL) is treated with 1.25M HCl/MeOH (0.6 mmol, 3.5 equiv.). A white crystalline solid precipitates which is filtered and dried (49 mg). ¹H NMR (400 MHz, D₂O) δ 7.57 (1H, d, J=7.2 Hz); 7.43 (1H, t, J=7.8 Hz); 7.18 (1H, t, J=7.4 Hz); 7.10 (1H, d, J=8.8 Hz); 4.22 (2H, t, J=6.4 Hz); 3.26 (2H, t, J=6.4 Hz); 2.86 (6H, s); 2.31 (3H, s). LCMS: Method B, Rₜ=2.93 min, [MH⁺=271].

### EXAMPLE 8

### Preparation of 3-Methyl-5-(2-morpholin-4-yl-ethyl)-1,5-dihydro-pyrazolo[4,3-c]quinolin-4-one

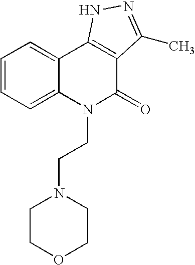

Prepared from the appropriate reagents by an analogous procedure to Example 7 ¹H NMR (400 MHz, DMSO-d₆) δ 8.11 (1H, dd, J=8.0, 1.2 Hz); 7.73 (1H, d, J=8.8 Hz); 7.54 (1H, t, J=7.2 Hz); 7.30 (1H, t, J=7.4 Hz); 4.62 (2H, t, J=7.6 Hz); 3.95 (2H, d, J=12.0 Hz); 3.72 (2H, t, J=12.0 Hz); 3.58 (2H, t, J=12.0 Hz); 3.30 (2H, m); 3.15 (2H, m); 2.52 (3H, s). LCMS: Method B, Rₜ=3.01 min, M+H⁺=313.

### EXAMPLE 9

### Preparation of 5-[3-(3,4-Dihydro-1H-isoquinolin-2-yl)-propyl]-3-methyl-1,5-dihydro-pyrazolo[4,3-c]quinolin-4-one

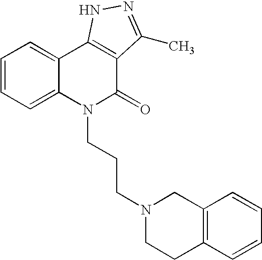

Prepared from the appropriate reagents by an analogous procedure to Example 1 ¹H NMR (400 MHz, DMSO-d₆); δ 7.94 (1H, dd, J=8.0, 1.2 Hz); 7.43 (1H, d, J=8.4 Hz); 7.36 (1H, m); 7.10 (1H, t, J=7.4 Hz); 7.07-6.97 (3H, m); 6.94 (1, m); 4.30 (1H, m); 4.2-4.1 (2H, t, J=7.0 Hz); 4.1-4.0 (1H, m); 3.46 (1H, m)l 3.2-3.0 (3H, m); 3.0-2.9 (1H, m); 2.77 (1H, m); 2.35 (3H, s); 1.97 (2H, quintet, J=7.3 Hz). LCMS: Method B, Rₜ=3.58 min, [MH⁺=373].

### EXAMPLE 10

### Preparation of 5-(2-Hydroxy-ethyl)-3-methyl-1,5-dihydro-pyrazolo[4,3-c]quinolin-4-one

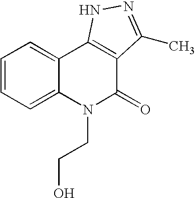

Prepared as follows:

### Steps 1 and 2 as Example 3

Remaining steps as Steps 2 and 3 in Example 1. ¹H NMR (400 MHz, DMSO-D₆); δ 8.10 (1H, dd, J=8.0, 1.2 Hz); 7.64 (1H, d, J=8.4 Hz); 7.56 (1H, m); 7.29 (1H, t, J=7.4 Hz); 4.91 (1H, t, J=6.8 Hz); 4.32 (2H, t, J=6.6 Hz); 3.63 (2H, m); 2.57 (3H, s).

LCMS: Method B, Rₜ=2.68 min, M+H⁺=244.

### EXAMPLE 11

### Preparation of 5-(2-Amino-ethyl)-3-methyl-1,5-dihydro-pyrazolo[4,3-c]quinolin-4-one

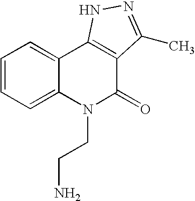

Prepared from Example 1 by the following procedure. [2-(3-Methyl-4-oxo-1,4-dihydro-pyrazolo[4,3-c]quinolin-5-yl)-ethyl]-carbamic acid tert-butyl ester (Example 1) (0.100 g, 0.29 mmol) is stirred in 1.25M HCl/MeOH (5 mL) at room temperature. If complete conversion is not observed within 24 h, the solvent is removed in vacuo and the treatment repeated. After concentration the solid is re-evaporated from methanol 5 times and dried. The hydrochloride salt is obtained as a white solid (0.080 g). ¹H NMR (400 MHz, DMSO-D₆); δ 8.03 (1H, dd, J=7.6, 1.2 Hz); 8.01-7.96 (3H, br s); 7.57 (1H, d, J=8.8 Hz); 7.43 (1H, m); 7.19 (1H, t, J=7.4 Hz); 4.38 (2H, t, J=6.8 Hz); 2.9 (2H, m); 2.43 (3H, s). LCMS: Method A, 5-55% B gradient. Rₜ=2.19 min, [MH⁺=243, M-(NH₂)=226].

### EXAMPLE 12

### Preparation of 5-(3-Benzyloxy-propyl)-3-methyl-1,5-dihydro-pyrazolo[4,3-c]quinolin-4-one

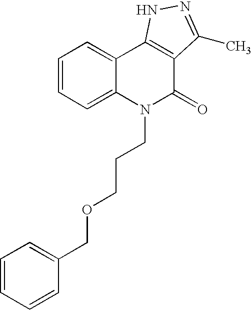

Prepared from the appropriate reagents by an analogous procedure to Example 1. ¹H NMR (400 MHz, DMSO-d₆); δ 8.11 (1H, m); 7.62-7.45 (2H, m); 7.40-7.23 (6H, m); 4.49 (2H, s); 4.32 (2H, m); 3.57 (2H, t, J=6.2 Hz); 2.63 (s) and 2.54 (s) 3H combined; 1.90 (2H, m). LCMS: Method A, Rₜ=2.99 min, [MH⁺=348].

### EXAMPLE 13

### Preparation of [3-(3-Methyl-4-oxo-1,4-dihydro-pyrazolo[4,3-c]quinolin-5-yl)-propyl]-carbamic acid benzyl ester

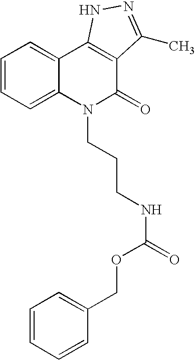

Prepared from the appropriate reagents by an analogous procedure to Example 1. ¹H NMR (400 MHz, DMSO-d₆); δ 8.12 (1H, d, J=7.6 Hz); 7.55 (2H, m); 7.4-7.3 (6H, m); 5.04 (2H, s); 4.27 (2H, t, J=7.4 Hz); 3.14 (2H, q, J=6.4 Hz); 2.58 (3H, s); 1.78 (2H, quintet, J=7.1 Hz). LCMS: Method B, Rₜ=3.49 min, [MH⁺=391].

### EXAMPLE 14

### Preparation of 5-(3-Hydroxy-propyl)-3-methyl-1,5-dihydro-pyrazolo[4,3-c]quinolin-4-one

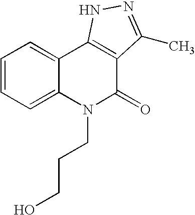 Prepared by hydrogenolysis of Example 12 as follows.

A degassed solution of 5-(3-benzyloxy-propyl)-3-methyl-1,5-dihydro-pyrazolo[4,3-c]quinolin-4-one (Example 12) (0.135 g, 0.39 mmol) in a DCM (2 mL)/ethanol (4 mL) mixture is treated with 5% wt:Pd/C (0.085 g, 0.04 mmol) and the suspension exposed to a hydrogen atmosphere. After stirring over night the reaction mixture is filtered through celite and washed through with methanol. The combined eluants are concentrated in vacuo providing an off white solid, which is taken up in ethyl acetate, filtered, washed with ethyl acetate and dried. The desired product is obtained as an off white solid (0.080 g, 80% yield). ¹H NMR (400 MHz, DMSO-d₆); δ 8.11 (1H, d, J=7.6 Hz); 7.6 (2H, br s); 7.3 (1H, br s); 4.67 (1H, t, J=7.2 Hz); 4.30 (2H, t, J=6.8 Hz); 3.53 (2H, q, J=6.8 Hz); 2.56 (3H, br s); 1.77 (2H, m). LCMS: Method A, 5-55% B. Rₜ=3.26 min, [MH⁺=258].

### EXAMPLE 15

### Preparation of 5-(3-Amino-propyl)-3-methyl-1,5-dihydro-pyrazolo[4,3-c]quinolin-4-one

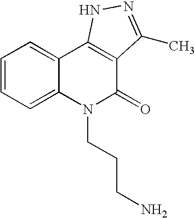

Prepared by acid hydrolysis of Example 6. Procedure as in Example 11. ¹H NMR (400 MHz, D₂O); δ 7.46 (1H, d, J=7.7 Hz); 7.39 (1H, t, J=7.7 Hz); 7.18-7.06 (2H, m); 3.97 (2H, t, J=6.8 Hz); 2.95 (2H, t, J=7.4 Hz); 2.34 (3H, s); 1.92 (2H, quintet, J=7.2 Hz).

LCMS: Method B, Rₜ=2.39 min, [MH⁺=257].

### EXAMPLE 16

### Preparation of 8-Chloro-5-(3-dimethylamino-propyl)-3-methyl-2,5-dihydro-pyrazolo[4,3-c]quinolin-4-one

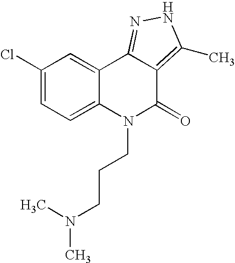

### Step 1 Preparation of 5-Chloro-2-(3-oxo-butyrylamino)-benzoic acid methyl ester

To a suspension of methyl 2-amino-5-chlorobenzoate (35.6 mmoles, 1 eq, 5 g) in toluene (40 ml) was added 2,2,2 trimethyl-1,3 dioxine-4-one (39.1 mmoles, 1.1 eq, 5.1 ml). The solution was refluxed for 24 hours and left to stand at RT over the weekend. The resultant precipitate was successively filtered, washed with toluene and dried to afford 5-chloro-2-(3-oxo-butyrylamino)-benzoic acid methyl ester as a beige solid (7.9 g, 83%). ¹H NMR (400 MHz, DMSO-d6) δ 10.62(1H, s, br); 8.16 (1H, d, J=9.0 Hz); 7.85 (1H, d, J=2.6 Hz); 7.67 (1H, dd, J=9.0, 2.6 Hz); 3.85 (3H, s); 3.68 (2, s); 2.22 (3H, s).

### Step 2 Preparation of 3-Acetyl-6-chloro-1H-quinoline-2,4-dione

To a suspension of 5-chloro-2-(3-oxo-butyrylamino)-benzoic acid methyl ester (29.5 mmoles, 1 eq, 7.96 g) in EtOH (500 ml) was added sodium ethoxide (118 mmoles, 4 eq, 8 g), the reaction mixture was refluxed for 12 hours. The obtained slurry was concentrated under vacuum and the residue suspended in H₂O (200 ml). The mixture was acidified to pH=2 with HCl 4M (20 ml), the formed precipitate was successively filtered, washed with H₂O (30 ml), Et₂O (2×100 ml) and dried to afford 3-Acetyl-6-chloro-1H-quinoline-2,4-dione as a white/beige solid (4.4 g, 63%). ¹H NMR (400 MHz, DMSO-d₆) δ 11.66 (1H, s, br); 7.92 (1H, s, br); 7.72 (1H, dd, J=8.8, 2.6 Hz); 7.32 (1H, d, J=8.8 Hz); 2.72 (3H, s).

### Step 3 Preparation of 3-Methyl-8-choro-2,5-dihydro-pyrazolo[4,3-c]quinolin-4-one

To a suspension of 3-acetyl-6-chloro-1H-quinoline-2,4-dione (18.4 mmoles, 1 eq, 4.36 g) in DMF (60 ml) was added hydrazine hydrate (46.4 mmoles, 3 eq, 1.45 ml), The resulting solution was refluxed for 4 hours. Upon completion the reaction mixture was left to cool standing overnight and the formed precipitate was successively filtered, washed with MeOH (3×15 ml), Et₂O (2×60 ml) and dried to afford 3-Methyl-8-nitro-2,5-dihydro-pyrazolo[4,3-c]quinolin-4-one as a white solid (4.03 g, 94%).

¹H NMR (400 MHz, DMSO-d6) δ 13.70 (1H, s, br); 11.22 (1H, s, br); 8.04 (1H, s, br); 8.04 (1H, s, br); 7.49 (1H, dd, J=8.9, 2.0 Hz); 7.35 (1H, d, J=8.8 Hz); 2.55 (3H, s).

### Step 4 Preparation of 8-Chloro-3-methyl-1-(tetrahydro-pyran-2-yl)-1,5-dihydro-pyrazolo[4,3-c]quinolin-4-one

To a suspension of 3-methyl-8-chloro-2,5-dihydro-pyrazolo[4,3-c]quinolin-4-one (17.1 mmoles, 1 eq, 4 g) in DMF (200 ml) was successively added 3,4-dihydro-2H-pyran (68.5 mmoles, 4 eq, 6.25 ml) and para-toluenesulfonic acid (1.7 mmoles, 0.1 eq, 323 mg). The mixture was heated at 90° C. for 2 days. The reaction mixture was concentrated under vacuum and the residue retreated with 20% of the above reagents under the conditions above for 4 hours. The obtained solution was left to stand and the formed precipitate was filtered and washed with MeOH/Et₂O to afford 3-Methyl-8-chloro-2-(tetrahydro-pyran-2-yl)-2,5-dihydro-pyrazolo[4,3-c]quinolin-4-one as a white solid (4.8 g, 88%). ¹H NMR (400 MHz, DMSO-d₆) δ 11.20 (1H, s); 7.92 (1H, d, J=2.4 Hz); 7.47 (1H, dd, J=8.8, 2.6 Hz); 7.32 (1H, d, J=8.6 Hz); 5.67 (1H, dd, J=9.6, 2.5 Hz); 3.94 (1H, d m, J=11.1 Hz); 3.68-3.76 (1H, m); 2.73 (3H, s); 2.36-2.46 (1H, m); 2.06 (1H, m); 1.97 (1H, m); 1.75 (1H, m); 1.59 (2H, m). LCMS: Method A, Rₜ=3.21 min, [MH⁺=318].

### Step 5 Preparation of 8-Chloro-5-(3-dimethylamino-propyl)-3-methyl-1-(tetrahydro-pyran-2-yl)-1,5-dihydro-pyrazolo[4,3-c]quinolin-4-one

8-Chloro-3-methyl-1-(tetrahydro-pyran-2-yl)-1,5-dihydro-pyrazolo[4,3-c]quinolin-4-one (200 mg, 0.63 mmol, 1 eq) was suspended in DMF (20 ml) in a round-bottomed flask and potassium carbonate (521 mg, 3.78 mmol, 6 eq) was added with vigorous stirring to enable sufficient mixing of the reaction. The reaction was heated to 60° C. with stirring before dimethylaminopropyl chloride hydrochloride (298 mg, 1.89 mmol, 3 eq) was added in one portion. The reaction was heated at 60° C. under nitrogen with vigorous stirring for 19 hours, and the heating increased to 90° C. for a further hour. The reaction mixture was filtered and the solid obtained was washed with DMF (5 ml). The washings and filtrate were combined and concentrated in vacuo. The residue thus obtained was partitioned between DCM (60 ml) and water (60 ml) and the aqueous washed with DCM (3×60 ml). The organic washings were combined, washed with water (30 ml) and brine (30 ml) before drying over anhydrous sodium sulphate and concentrating in vacuo. The solid thus obtained was purified using flash column chromatography (5:5:1 methanol:dichloromethane:7N Ammonia solution in methanol) to afford the title compound 8-Chloro-5-(3-dimethylamino-propyl)-3-methyl-1-(tetrahydro-pyran-2-yl)-1,5-dihydro-pyrazolo[4,3-c]quinolin-4-one as a light brown solid (173 mg, 68%). ¹H NMR (400 MHz, CDCl₃) δ 8.26 (d, 1H, J=2.6 Hz); 7.41 (dd, 1H, J=9.1, 2.4 Hz); 7.34 (d, 1H, J=9.1 Hz); 5.52 (dd, 1H, J=9.2, 2.7 Hz); 4.30 (t, 2H, J=7.6 Hz); 4.08 (d, 1H, J=11.7 Hz); 3.71 (dt, 1H, J=11.1, 2.9 Hz); 2.84 (s, 3H); 2.62 (m, 1H); 2.54 (m, 2H); 2.34 (s, 6H); 2.20 (m, 1H); 2.04 (m, 1H); 1.96 (m, 2H), 1.78 (m, 2H), 1.66 (m, 1H). LCMS, Method D, 40-100% Rₜ=9.27 min [MH⁺=403].

### Step 6

8-Chloro-5-(3-dimethylamino-propyl)-3-methyl-1-(tetrahydro-pyran-2-yl)-1,5-dihydro-pyrazolo[4,3-c]quinolin-4-one (192 mg, 0.48 mmol) was dissolved in 1.25M HCl in methanol (10 ml) and stirred at ambient temperature for 1 hour. A white precipitate was seen forming after five minutes. The reaction mixture was concentrated, dissolved in methanol (5 ml) and concentrated. The off-white solid formed was washed with diethyl ether (2×5 ml) and dried to give 8-Chloro-5-(3-dimethylamino-propyl)-3-methyl-2,5-dihydro-pyrazolo[4,3-c]quinolin-4-one hydrochloride as an off-white solid (149 mg, 98%). ¹H NMR (400 MHz, DMSO-d₆) δ 10.2 (s, br); 8.2 (d, 1H, J=2.4 Hz); 7.7 (d, 1H, J=9.3 Hz); 7.6 (dd, 1H, J=9.1, 2.3 Hz); 4.3 (t, 2H, J=7.3 Hz); 3.2 (m, 2H); 2.7 (2 peaks, 6H); 2.6 (s, 3H); 2.1 (quintet, 2H, J=7.5 Hz).

### EXAMPLE 17

### Preparation of 4-(8-Bromo-3-methyl-4-oxo-1,4-dihydro-pyrazolo[4,3-c]quinolin-5-yl)-butyric acid methyl ester

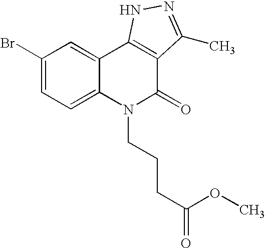

### Steps 1,2,3,4

Using a similar protocol as in Example 16, with the appropriate reagents in steps 1, 2, 3 lead to 8-Bromo-3-methyl-1-(tetrahydro-pyran-2-yl)-1,5-dihydro-pyrazolo[4,3-c]quinolin-4-one. ¹H NMR (400 MHz, DMSO-d₆) δ 11.19(1H, s); 8.06(1H, d, J=2.2 Hz); 7.59(1H, dd, J=8.8, 2.4 Hz); 7.26(1H, d, J=8.8 Hz); 5.67(1H, dd, J=9.5, 2.4 Hz); 3.93(1H, d m, J=11.7 Hz); 3.68 to 3.76(1H, m); 2.71(3 h, s); 2.36 to 2.46(1H, m); 2.06(1H. m); 1.98(1H, m); 1.74(1H, m); 1.59(2H, m) LCMS, condition A, Rₜ=3.29 min, [MH⁺=364].

### Step 5 Preparation of 4-[8-Bromo-3-methyl-4-oxo-1-(tetrahydro-pyran-2-yl)-1,4-dihydro-pyrazolo[4,3-c]quinolin-5-yl]-butyric acid methyl ester

8-Bromo-3-methyl-1-(tetrahydro-pyran-2-yl)-1,5-dihydro-pyrazolo[4,3-c]quinolin-4-one (500 mg, 1.38 mmol, 1 eq) was suspended in DMF (10 mL) and heated to 60° C. to aid dissolution. Potassium carbonate (1.145 g, 8.29 mmol, 6 eq) was added with stirring followed by methyl-4-bromobutyrate (750 mg, 4.14 mmol, 3 eq) in one portion. The reaction mixture was stirred at 60° C. under nitrogen for 27 hours. The reaction mixture was concentrated in vacuo and the residue partitioned between DCM (60 ml) and H₂O (60 ml) and washed with DCM (3×50 ml). The organic layers were combined and washed with water (30 ml) and brine (30 ml) before drying over anhydrous sodium sulphate and concentration to give a yellow solid. This solid (mixture of o-alkylated and N-alkylated derivatives) was purified by flash column chromatography (30-50% ethyl acetate in iso-hexane) to afford 4-[8-Bromo-3-methyl-4-oxo-1-(tetrahydro-pyran-2-yl)-1,4-dihydro-pyrazolo[4,3-c]quinolin-5-yl]-butyric acid methyl ester as a white solid (367 mg, 57%). ¹H NMR (400 MHz, CDCl₃) δ 8.41 (d, 1H, J=2.4Hz); 7.88 (dd, 1H, J=9.1, 2.4 Hz); 7.40 (d, 1H, J=9.1 Hz); 5.52 (dd, 1H, J=9.4, 2.7 Hz); 4.29 (t, 1H, J=7.7 Hz); 4.08 (m, 1H); 3.72 (m, 1H); 3.71 (s, 3H); 2.83 (s, 3H); 2.60 (m, 1H); 2.50 (t, 2H, J=7.0 Hz); 2.22 (m, 1H); 2.04 (m, 2H); 2.02 (m, 1H); 1.77 (m, 2H); 1.65 (m, 1H). LCMS: (formic acid 5-100% 5 min) Rₜ=3.86 min [MH⁺=462/464].

### Step 6

4-[8-Bromo-3-methyl-4-oxo-1-(tetrahydro-pyran-2-yl)-1,4-dihydro-pyrazolo[4,3-c]quinolin-5-yl]-butyric acid methyl ester (120 mg) was treated with a 1:1 solution of trifluoroacetic acid:water (5 ml) at room temperature. The mixture was stirred for 6 minutes and concentrated under vacuum. Several co-evaporations with 4M HCl in dioxane afforded a white solid which was successively washed with Et₂O and dried to afford the title compound 4-(8-Bromo-3-methyl-4-oxo-1,4-dihydro-pyrazolo[4,3-c]quinolin-5-yl)-butyric acid methyl ester as a white solid (80 mg, 82%).

¹H NMR (400 MHz, DMSO-d₆) δ 8.29 (1H, d, J=2.4 Hz); 7.71 (1H, dd, J=9.1, 2.4 Hz); 7.60 (1H, d, J=9.1 Hz); 4.23 (2H, t, J=7.6 Hz); 3.58 (3H, s); 2.57 (3H, s); 2.45 (2H, t, J=7.3 Hz); 1.85 (2H, quintet, J=7.3 Hz). LCMS, Method B, Rₜ=3.93, [MH+=378].

### EXAMPLE 18

### Preparation of 8-Bromo-5-(3-dimethylamino-propyl)-3-methyl-2,5-dihydro-pyrazolo[4,3-c]quinolin-4-one

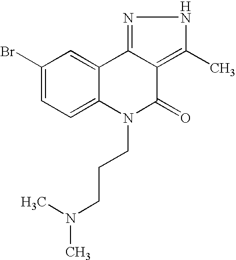

Using the appropriate reagents an in a similar manner as for Examples 16 and 17, the title compound was obtained after 6 steps as a white solid. ¹H NMR (400 MHz, DMSO-d6) δ 10.14 (s, 1H, broad); 8.33 (d, 1H, J=2.0 Hz); 7.71 (dd, 1H, J=9.1 Hz, 2.0 Hz); 7.60 (d, 1H, J=9.1 Hz); 4.30 (t, 2H, J=7.3 Hz); 3.19-3.13 (m, 2H); 2.75 (s, 6H, broad); 2.58 (s, 3H); 2.51-2.49 (m, 2H). LCMS Method D, 20-100%, Rₜ=8.41 min [MH+=363].

### EXAMPLE 19

### Preparation of 4-(8-Bromo-3-methyl-4-oxo-1,4-dihydro-pyrazolo[4,3-c]quinolin-5-yl)-butyric acid

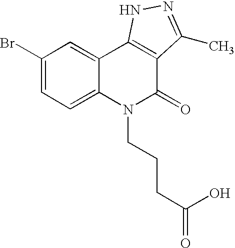

4-[8-Bromo-3-methyl-4-oxo-1-(tetrahydro-pyran-2-yl)-1,4-dihydro-pyrazolo[4,3-c]quinolin-5-yl]-butyric acid methyl ester (example 17) (350 mg, 0.76 mmol) was treated with a 1:1 solution of trifluoroacetic acid: water (5 ml) at 50° C. for four hours, then concentrated in vacuo. The residue was dissolved in DCM and concentrated in vacuo. This dissolution procedure with DCM was repeated three times. The residue was then treated with 4M HCl in Dioxan and concentrated in vacuo three times and the solid thus obtained was washed with diethyl ether and dried in vacuo to afford the title compound 4-(8-Bromo-3-methyl-4-oxo-1,4-dihydro-pyrazolo[4,3-c]quinolin-5-yl)-butyric acid as a beige solid (267 mg, yield 97%). ¹H NMR (400 MHz, DMSO-d6) δ 8.28 (d, 1H, J=2.2 Hz); 7.70 (dd, 1H, J=9.0, 2.4 Hz); 7.62 (d, 1H, J=9.1 Hz); 4.23 (t, 2H, J=7.5Hz); 2.57 (s, 3H); 2.36 (t, 2H, J=7.2 Hz); 1.82 (quintet, 2H, J=7.5 Hz). LCMS, Method C, 20-100%, Rₜ=5.81 min [MH⁺=364/366].

### EXAMPLE 20

### Preparation of 4-(8-Bromo-3-methyl-4-oxo-2,4-dihydro-pyrazolo[4,3-c]quinolin-5-yl)-butyronitrile

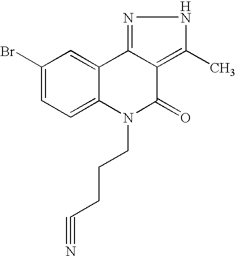

Using the appropriate reagents and in a similar manner as for Example 17, the title compound was obtained after 6 steps as a white solid. ¹H NMR (400 MHz, DMSO-d6) δ 13.76 (s, 1H, broad); 8.28 (s, 1H, broad); 7.70 (dd, 1H, J=9.1, 2.3); 7.55 (d, 1H, J=9.1); 4.30 (t, 2H, J=7.3); 2.63 (t, 2H, J=7.1); 2.57 (s, 3H); 1.94-1.86 (m, 2H). LCMS Method C, 20%-100%, Rₜ=6.72 min [MH+=345/347].

### EXAMPLE 21

### Preparation of 4-[4-(8-Bromo-3-methyl-4-oxo-1,4-dihydro-pyrazolo[4,3-c]quinolin-5-yl)-butyryl]-piperazine-1-carboxylic acid tert-butyl ester

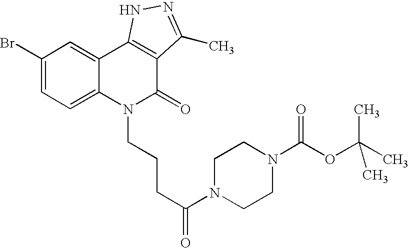

4-(8-Bromo-3-methyl-4-oxo-1,4-dihydro-pyrazolo[4,3-c]quinolin-5-yl)-butyric acid (Example 19) (60 mg, 0.16 mmol) and 1-hydroxy-1H-benzotriazole hydrate (33 mg, 0.25 mmol) were dissolved in DMF (2.0 ml) with stirring. Upon dissolution, N,N-Diisopropyl ethylamine (54 uL, 0.49 mmol) and N-Boc-piperazine (92 mg, 0.49 mmol, 3 eq) were added. The reaction was stirred and 1-ethyl-3-(3-dimethylaminopropyl)carbodiimide (47 mg, 0.25 mmol) was added in one portion. The reaction was stirred at ambient (room) temperature for 68 hours. The mixture was concentrated in vacuo and the residue partitioned between ethyl acetate (200 ml) and water (40 ml). The organic layer was then washed with 0.2M HCl (aqueous) (6×30 ml), water (30 ml), sodium bicarbonate (saturated aqueous) (4×30 ml), and water (4×30 ml). The organic solution was concentrated in vacuo to yield an orange solid, which was washed with diethyl ether and dried in vacuo to yield the title compound (67 mg, yield=76%). ¹H NMR (400 MHz, CDCl₃) δ 8.55 (d, 1H, J=2.2 Hz); 7.86 (d, 1H, J=9.3 Hz); 7.78 (dd, 1H, J=9.3, 2.6 Hz); 4.34 (t, 2H, J=7.8 Hz); 3.63 (m, 2H); 3.40-3.55 (m, 6H); 2.91 (s, 3H); 2.52 (t, 2H, J=6.3 Hz); 2.60 (m, 2H); 1.48 (s, 9H). LCMS, Method C, 40-100% Rₜ=5.98 min [MH⁺=543, MH⁻=531].

### EXAMPLE 22

### Preparation of 4-(8-Bromo-3-methyl-4-oxo-1,4-dihydro-pyrazolo[4,3-c]quinolin-5-yl)-N-(4-chloro-benzyl)-butyramide

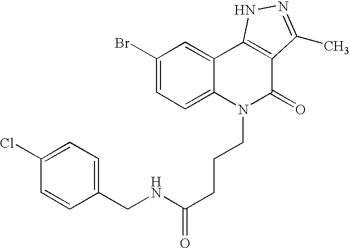

From Example 19 and in a similar manner as for Example 21, the title compound was obtained as a white solid. ¹H NMR (400 MHz, DMSO-d₆) δ 8.40 (1H, t, J=5.8 Hz); 8.27 (1H, s, br); 7.68 (1H, dd, J=9.1, 1.7 Hz); 7.58 (1H, d, J=9.1 Hz); 7.36 (2H, d, J=8.4 Hz); 7.27 (2H, d, J=8.4 Hz); 4.25 (1H, d, J=5.9 Hz); 4.22 (2H, quartet, J=7.9 Hz); 2.57 (3H, s); 2.29 (2H, t, J=7.2 Hz); 1.84 (2H, quintet, J=7.4 Hz). LCMS, Method A, Rₜ=3.08 min [MH+=489].

### EXAMPLE 23

### Preparation of 5-(2-Amino-3-phenyl-propyl)-3-methyl-1,5-dihydro-pyrazolo[4,3-c]quinolin-4-one

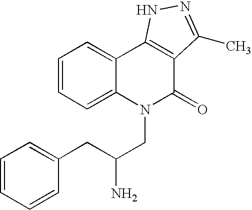

Using the appropriate reagent and in a similar manner as for Example 1, and hydrolysis of intermediate Boc-protected derivative as in Example 11, the title compound was obtained as a white solid. ¹H NMR (400 MHz, D₂O) δ 7.50 (1H, d, J=8.1 Hz); 7.26 (3H, m); 7.15-7.0 (4H, m); 5.94 (1H, d, J=7.7 Hz); 4.15 (1H, dd, J=15.5, 8.7 Hz); 3.61 (1H, m); 3.33 (1H, br d, J=15.4 Hz); 3.05 (1H, dd, J=13.7, 4.4 Hz); 2.80 (1H, dd, J=13.5, 10.9 Hz); 2.29 (3H, s). LCMS, Method B, Rₜ=3.33 min, [MH+]=333.

### EXAMPLE 24

### Preparation of [3-(3-Methyl-4-oxo-4,5-dihydro-2H-pyrazolo[4,3-c]quinolin-6-yloxy)-propyl]-carbamic acid tert-butyl ester

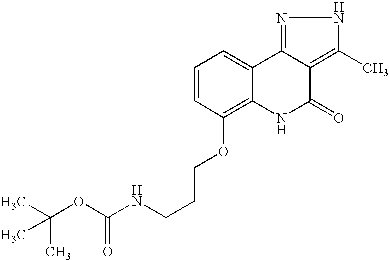

Using the appropriate reagents and in manner similar to that exemplified in Steps 1 to 3 of Example 55, the title compound was obtained as a white solid after recrystallisation. ¹H NMR (400 MHz, DMSO-d6) δ appears as a mixture isomers 80/20: 13.76(0.8H, broad); 13.65(0.2H); 10.07(0.8H, broad); 9.71(0.2H, broad); 7.61-7.57(2d, 1H, J=7.7); 7.18(d, 0.2H, J=8.0);7.16(dₐₚₚₐᵣₑₙₜ, 0.8H, J=8.1); 7.04(dₐₚₚₐᵣₑₙₜ, 0.2H, J=8.0); 6.97(tₐₚₚₐᵣₑₙₜ, 0.8H, J=5.6); 4.13(tₐₚₚₐᵣₑₙₜ, 2H, J=5.4); 3.17-3.13(m, 2H); 2.63(s, 0.6H); 2.53(s, 2.4H); 1.98-1.90, 2H); 1.336(s, 9H). LCMS Method B, Rₜ=3.40 min [MH+=373].

### EXAMPLE 25

### Preparation of 6-(3-Amino-propoxy)-3-methyl-2,5-dihydro-pyrazolo[4,3-c]quinolin-4-one

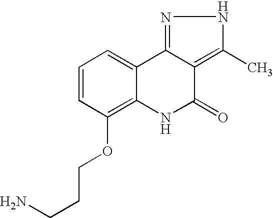

[3-(3-Methyl-4-oxo-4,5-dihydro-2H-pyrazolo[4,3 -c]quinolin-6-yloxy)-propyl]-carbamic acid tert-butyl ester (Example 24) (31 mg) was treated with a 4M solution of HCl in dioxan for 1 hour. Upon completion the reaction mixture was concentrated and the resulting residue washed with MeOH (1 ml) and Et₂O and dried to afford the title compound as white solid (25 mg). ¹H NMR (400 MHz, DMSO-d6) δ 10.161(s, 1H); 7.99(s, 3H, broad); 7.64(d, 1H, J=7.7); 7.18(dd, 1H, J=8.0, 7.7); 7.09(d, 1H, J=8.05); 4.21(t, 2H, J=5.6); 3.13-3.08(m, 2H); 2.56(s, 3H); 2.15-2.09(m, 2H). LCMS Method B, Rₜ=2.33 min [MH+=273/274].

### EXAMPLE 26 AND 27 NOT INCLUDED TO FACILITATE RENUMBERING

### EXAMPLE 28

### Preparation of 3-(4-Fluoro-phenyl)-1,5-dihydro-pyrazolo[4,3-c]quinolin-4-one

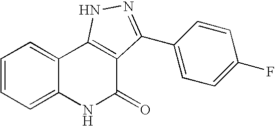

Using the appropriate reagent and in manner similar to that exemplified in Example 26, the title compound was obtained as a white solid. ¹H NMR (400 MHz, DMSO-d6) δ 8.33 (2H, m); 8.03 (1H, m); 7.44 (1H, t, J=7.6 Hz); 7.34 (1H, d, J=8. Hz); 7.27-7.12 (3H, m). LCMS, Method B, Rₜ=3.28 min, [MH+=280].

### EXAMPLE 29

### Preparation of [4-(8-Bromo-3-methyl-4-oxo-1,4-dihydro-pyrazolo[4,3-c]quinolin-5-yl)-butyl]-carbamic acid tert-butyl ester

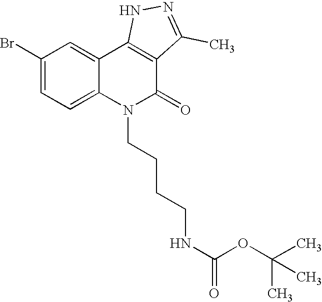

### Step 1: Preparation of [4-(6-Bromo-2,4-dioxo-4H-benzo[d][1,3]oxazin-1-yl)-butyl]-carbamic acid tert-butyl ester

A slurry of 5-bromoisatoic anhydride (2.42 g, 10 mmol, 1 equiv.), triphenylphosphine (3.41 g, 13 mmol, 1.3 equiv.) and 4-(tert-butoxycarbonylamino)-1-butanol (2.46 g, 13 mmol, 1.3 equiv.) in THF (100 mL) is treated dropwise with diisopropylazodicarboxylate (2.56 mL, 13 mmol, 1.3 equiv.) providing a yellow solution. After 18 h the solvent is removed in vacuo providing a yellow gum. Purification by silica gel chromatography (25%-33%-50% EtOAc/isohexane gradient) provides 3.28 g (79% yield) of white solid, ˜80% pure. ¹H NMR (400 MHz, DMSO-d₆) δ 8.08 (1H, d, J=2.3 Hz); 7.97 (1H, dd, J=9.0, 2.2 Hz); 7.48 (1H, d, J=8.9 Hz); 6.83 (1H, m); 3.99 (2H, t, J=7.5 Hz); 2.94 (2H, q, J=6.0 Hz); 1.60 (2H, m); 1.47 (2H, m); 1.36 (9H, s).

### Step 2: Preparation of 5-Bromo-2-(4-tert-butoxycarbonylamino-butylamino)-benzoic acid methyl ester

Sodium hydroxide (0.64 g, 16 mmol, 2 equiv.) is dissolved in methanol (40 mL) and the solution treated with [4-(6-bomo-2,4-dioxo-4H-benzo[d][1,3]oxazin-1-yl)-butyl]-carbamic acid tert-butyl ester (3.2 g, 7.9 mmol, 1 equiv.) before heating at 70° C. for 2 h. After cooling the solution is quenched with 1M HCl_{(aq)} and partitioned between water and ethyl acetate. The aqueous phase is washed with water and brine, dried over Na₂SO₄, filtered and concentrated. The crude material is purified by silica gel chromatography (DCM-5% EtOAc/DCM) to provide the desired product as a pale yellow solid (2.08 g, 66% yield). ¹H NMR (400 MHz, CDCl₃) δ 8.01 (1H, m); 7.71 (1H, m); 7.41 (1H, m); 6.57 (1H, d, J=8.9 Hz); 4.57 (1H, br s); 3.87 (3H, s); 3.20 (4H, m); 1.72 (2H, m); 1.64 (2H, m); 1.46 (9H, s).

### Step 3: Preparation of [4-(3-Acetyl-6-bromo-4-hydroxy-2-oxo-2H-quinolin-1-yl)-butyl]-carbamic acid tert-butyl ester

A solution of 5-Bromo-2-(4-tert-butoxycarbonylamino-butylamino)-benzoic acid methyl ester (2.05 g, 5.1 mmol) and 2,2,6-trimethyl-1,3-dioxin-4-one (0.735 mL, 5.6 mmol) in toluene (10 mL) is microwave irradiated at 140° C. for 600 s. After removal of the solvent in vacuo the residue is purified by silica gel chromatography (1:1 EtOAc/hexane) providing 1.78 g (72% yield) of a pale yellow oil.

The intermediate (1.78 g, 3.67 mmol) is dissolved in ethanol (30 mL) and treated with sodium ethoxide (1.00 g, 14.7 mmol) and the solution heated at reflux for 2 h. After cooling 1M HCl_{(aq)} (15 mL) is added to pH 2, and the solvent removed in vacuo. The resulting orange gum is triturated in ether/water providing, after standing over night, a pale orange solid which is washed with water twice then ether twice. The dried product is a pale orange solid (1.15 g, 69% yield). ¹H NMR (400 MHz, CDCl₃) δ 8.32 (1H, d, J=2.3 Hz); 7.73 (1H, dd, J=8.9, 2.5 Hz); 7.20 (1H, t, J=9.4 Hz); 4.69 (1H, br s); 4.20 (2H, t, J=7.5 Hz); 3.21 (2H, q, J=6.2 Hz); 2.82 (3H, s); 1.72 (2H, m); 1.64 (2H, m); 1.44 (9H, s).

### Step 4: Preparation of [4-(8-Bromo-3-methyl-4-oxo-1,4-dihydro-pyrazolo[4,3-c]quinolin-5-yl)-butyl]-carbamic acid tert-butyl ester

A solution of [4-(3-acetyl-6-bromo-4-hydroxy-2-oxo-2H-quinolin- 1-yl)-butyl]-carbamic acid tert-butyl ester (0.453 g, 1 mmol) and hydrazine hydrate (0.180 mL, 3 mmol) in DMF (2.5 mL) is microwave irradiated at 200° C. for 300 s. The cooled solution is loaded onto a silica column and eluted with 3:1 EtOAc/hexane providing the desired product as a white solid (0.342 g, 76% yield). ¹H NMR (400 MHz, DMSO-d6) δ 8.27 (1H, s); 7.68 (1H, dd, J=9.0, 1.8 Hz); 7.53 (1H, d, J=9.1 Hz); 6.84 (1H, t, J=5.5 Hz); 4.20 (2H, t, J=6.8 Hz); 2.95 (2H, m); 2.57 (3H, s); 1.56 (2H, m); 1.46 (2H, m); 1.36 (9H, s). LCMS, Method B, Rₜ=3.71 min, [MH+/MNa+=471/473].

### EXAMPLE 30

### Preparation of 3-(8-Bromo-3-methyl-4-oxo-1,4-dihydro-pyrazolo[4,3-c]quinolin-5-ylmethyl)-piperidine-1-carboxylic acid tert-butyl ester

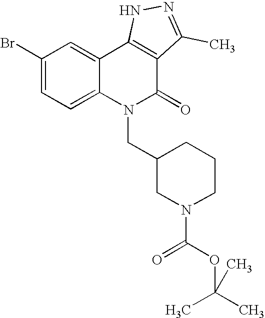

Using the appropriate reagents and in manner similar to that exemplified in Example 29 the title compound was obtained as a white solid. ¹H NMR (400 MHz, DMSO-d6) δ 13.79 (1H, s); 8.28 (1H, br s); 7.69 (1H, d, J=8.5 Hz); 7.53 (1H, d, J=8.8 Hz); 4.5-4.0 (2H, m); 3.9-3.5 (2H, m); 2.8-2.6 (2H, m); 2.58 (3H, s); 1.84 (1H, br s); 1.76 (1H, br s); 1.4-1.0 (11H, m). LCMS, Method B, Rₜ=3.93 min, [MH+=475/477].

### EXAMPLE 31

### Preparation of [3-(8-Chloro-3-methyl-4-oxo-1,4-dihydro-pyrazolo[4,3-c]quinolin-5-yl)-propyl]-carbamic acid tert-butyl ester

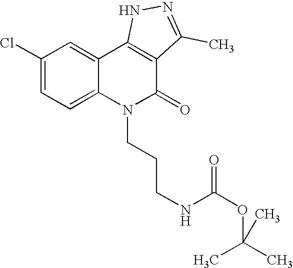

Using the appropriate reagents and in manner similar to that exemplified in Example 29 the title compound was obtained as a white solid. ¹H NMR (400 MHz, DMSO-d₆) δ 8.14 (1H, s); 7.58 (2H, s); 6.91 (1H, t, J=5.5 Hz); 4.22 (2H, m); 3.02 (2H, m); 2.58 (3H, s); 1.72 (2H, m); 1.38 (9H, s). LCMS, Method B, Rₜ=3.67 min, 389/391 (ES−, M−H).

### EXAMPLE 32

### Preparation of 5-(3-Amino-propyl)-8-chloro-3-methyl-1,5-dihydro-pyrazolo[4,3-c]quinolin-4-one

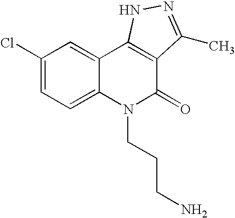

Boc deprotection of Example 31 in a manner similar to that exemplified in Example 11 affords the title compound as a white solid. ¹H NMR (400 MHz, D₂O) δ 7.18 (1H, dd, J=9.2, 2.4 Hz); 7.12 (1H, d, J=2.4 Hz); 7.01 (1H, d, J=9.2 Hz); 3.94 (2H, t, J=7.0 Hz); 2.94 (2H, t, J=7.4 Hz); 2.37 (3H, s); 1.88 (2H, quintet, J=7.3 Hz). LCMS, Method B, Rₜ=2.73 min, [MH+=291/293 274 (ES+, M-NH₂)].

### NO EXAMPLE 33

### EXAMPLE 34

### Preparation of 5-(4-Amino-butyl)-8-bromo-3-methyl-1,5-dihydro-pyrazolo[4,3-c]quinolin-4-one

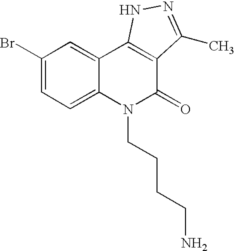

Boc deprotection of Example 29 in a manner similar to that exemplified in Example 11 affords the title compound as a white solid. ¹H NMR (400 MHz, D₂O) δ 7.18 (1H, dd, J=9.2, 2.4 Hz); 7.13 (1H, d, J=2.1 Hz); 6.78 (1H, d, J=9.1 Hz); 0.66 (2H, t, J=7.5 Hz); 2.92 (2H, t, J=7.8 Hz); 2.31 (3H, s); 1.60 (2H, m). LCMS: Method B, Rₜ=2.91 min, [MH+=349/351].

### EXAMPLE 35

### Preparation of 3-Methyl-N-[3-(3-methyl-4-oxo-2,4-dihydro-pyrazolo[4,3-c]quinolin-5-yl)-propyl]-butyramide

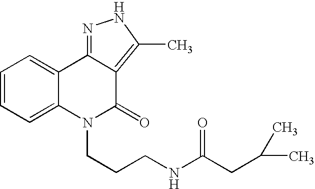

Prepared from Example 15 by the following procedure.

Dry DMF (1.5 ml) and DIPEA (200 ul) were added to 5-(3-Amino-propyl)-3-methyl-1,5-dihydro-pyrazolo[4,3-c]quinolin-4-one (Example15) (52.5 mg, 0.179 mmol) and the mixture was sonicated and then stirred for 15 min to produce a white suspension. A solution of isovaleroyl chloride (33 ul, 0.269 mmole) in dry DMF (0.5 ml) was slowly added to the vigorously stirred suspension of the amine, whereupon the suspension very quickly cleared. After mixing for 2 hr, tris-(2-aminoethyl)-amine polystyrene (Novabiochem, 200-400 mesh, ca. 0.34 mmole/g, ca. 100 mg, swelled in DCM and washed with DCM then DMF) was added to the reaction mixture and stirring continued for about 1 hr. The scavenger resin was removed by filtration and rinsed with DMF then ethyl acetate and the combined organic filtrates were distributed between ethyl acetate (180 ml) and water (50 ml). The separated organic layer was washed with 30-50 ml portions of water, 4-times; 0.2M HCl, 3 times; water, 1 time; saturated NaHCO₃, 4-times and then with water, 4-times. After the evaporation of the ethyl acetate and re-evaporation from methanol, 2-times, the target amide was treated 3-times with ether and dried in vacuo to give the pure title compound as white solid (40 mg, 65.6%). ¹H NMR (400 MHz, DMSO-d₆) δ 13.70 (1H, br s); 8.10 (1H, dd, J=7.7 Hz); 7.86 (1H, t, J=5.9 Hz); 7.50-7.60 (2H, m); 7.31 (1H, t, J=7.1 Hz); 4.23 (2H, t, J=7.1 Hz); 3.36 (2H, q, J=6.5 Hz); 2.58 (3H, s); 1.97 (3H, m); 1.75 (2H, quintet, J=7.3 Hz); 0.88 (6H, d, J=6.0 Hz). LCMS: Method D, 40-100%, Rₜ=8.46 min [MH+=341.31; MNa+=363.31].

### EXAMPLE 36

### Preparation of N-[4-(8-Bromo-3-methyl-4-oxo-1,4-dihydro-pyrazolo[4,3-c]quinolin-5-yl)-butyl]-benzamide

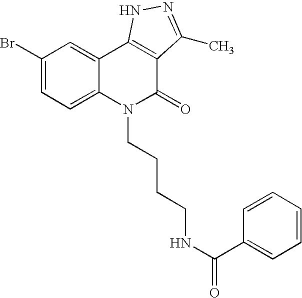

Using the appropriate reagent and in manner similar to that exemplified in Example 35 the title compound was obtained as a white solid. ¹H NMR (400 MHz, DMSO-d6) δ 8.55 (1H, t, J=5.6 Hz); 7.88 (2H, d, J=7.1 Hz); 7.8-7.6 (4H, m); 7.58 (2H, t, J=7.2 Hz); 7.51 (2H, t, J=7.5 Hz); 4.32 (2H, m); 2.72-2.56 (2H, m); 2.57 (3H, s); 1.69 (4H, m). LCMS: Method B, Rₜ=3.60 min, [MH+=453/455].

### EXAMPLE 37

### Preparation of 4-(8-Bromo-3-methyl-4-oxo-1,4-dihydro-pyrazolo[4,3-c]quinolin-5-ylmethyl)-piperidine-1-carboxylic acid tert-butyl ester

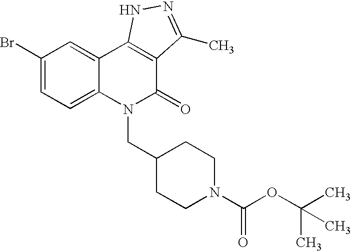

Using the appropriate reagent and in manner similar to that exemplified in Example 29 the title compound was obtained as a white solid.

¹H NMR (400 MHz, DMSO-d6) δ 13.8 (1H, br s); 8.27 (1H, br s); 7.68 (1H, dd, J=9.0, 1.8 Hz); 7.58 (1H, d, J =9.1 Hz); 4.17 (2H, m); 3.91 (2H, m); 2.7-2.5 (2H, m); 2.57 (3H, s); 1.96 (1H, m); 1.53 (2H, m); 1.39 (9H, s); 1.17 (2H, m). LCMS: Method B, Rₜ=4.19 min, [MH+=475/477].

### NO EXAMPLE 38

### EXAMPLE 39

### Preparation of 8-Bromo-3-methyl-5-piperidin-4-ylmethyl-1,5-dihydro-pyrazolo[4,3-c]quinolin-4-one

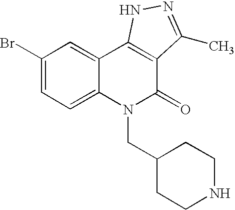

Boc deprotection of Example 37 in a manner similar to that exemplified in Example 11 affords the title compound as a white solid. ¹H NMR (400 MHz, D₂O) δ 7.43 (1H, br s); 7.30 (1H, br d, J=9.2 Hz); 6.93 (1H, d, J=9.2 Hz); 3.78 (2H, m); 3.22 (2H, d, J=12.8 Hz); 2.67 (2H, t, J=7.1 Hz); 2.35 (3H, s); 1.88 (1H, m); 1.63 (2H, m); 1.37 (2H, m). LCMS: Method B, Rₜ=2.92 min, [MH+=375/377].

### EXAMPLE 40

### Preparation of 4-Chloro-N-[3-(8-chloro-3-methyl-4-oxo-1,4-dihydro-pyrazolo[4,3-c]quinolin-5-yl)-propyl]-benzamide

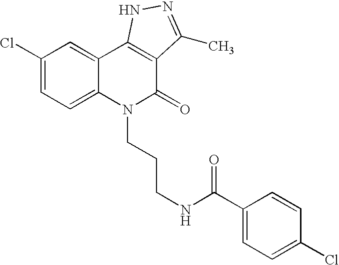

Starting from Example 32 and using the appropriate reagents and in manner similar to that exemplified in Example 35, the title compound was obtained as a white solid. ¹H NMR (400 MHz, DMSO-d6) δ 13.7 (1H, br s); 8.54 (1H, t, J=5.5 Hz); 8.08 (1H, br s); 7.80 (2H, d, J=8.4 Hz); 7.54 (2H, m); 7.48 (2H, d, J=8.4 Hz); 4.25 (2H, t, J=7.2 Hz); 3.31 (2H, q, J=6.5 Hz); 2.52 (3H, s); 1.82 (2H, quintet, J=7.1 Hz). LCMS: Method B, Rₜ=3.99, [MH+=429].

### EXAMPLE 41

### Preparation of [3-(8-Bromo-3-methyl-4-oxo-1,4-dihydro-pyrazolo[4,3-c]quinolin-5-yl)-2-methyl-propyl]-carbamic acid tert-butyl ester

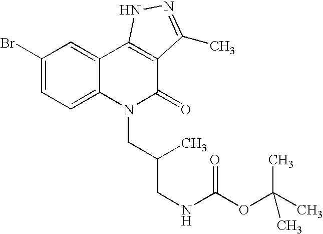

Using the appropriate reagent and in manner similar to that exemplified in Example 29 the title compound was obtained as a white solid. ¹H NMR (400 MHz, DMSO-d6) δ 13.78 (1H, s); 8.28 (1H, s); 7.67 (1H, d, J=8.6 Hz); 7.48 (1H, d, J=9.0 Hz); 6.84 (1H, m); 4.13 (2H, m); 2.99-2.92 (1H, m); 2.88-2.80 (1H, m); 2.58 (3H, s); 2.09 (1H, m); 1.36 (9H, s); 0.83 (3H, d, J=6.6 Hz). LCMS: Method B, Rₜ=4.05 min, [MNa+=471/473].

### EXAMPLE 42

### Preparation of 5-(3-Amino-2-methyl-propyl)-8-bromo-3-methyl-1,5-dihydro-pyrazolo[4,3-c]quinolin-4-one

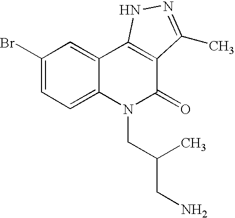

Boc deprotection of Example 41 in a manner similar to that exemplified in Example 11 affords the title compound as a white solid. ¹H NMR (400 MHz, DMSO-D₆) δ 8.34 (1H, d, J=2.2 Hz); 7.94 (3H, br s); 7.70 (1H, dd, J=9.0, 2.2 Hz); 7.59 (1H, d, J=9.2 Hz); 4.35-4.29 (1H, m); 4.13-4.09 (1H, m); 2.85-2.60 (4H, m); 2.58 (3H, s); 2.40-2.20 (1H, m); 1.01 (3H, d, J=6.5 Hz). LCMS: Method B, Rₜ=2.89 min, [MH+=349/].

### EXAMPLE 43

### Preparation of [3-(8-Bromo-3-methyl-4-oxo-1,4-dihydro-pyrazolo[4,3-c]quinolin-5-yl)-propyl]-carbamic acid benzyl ester

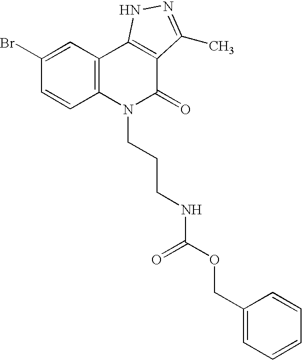

Using the appropriate reagent and in manner similar to that exemplified in Example 29 the title compound was obtained as a white solid. ¹H NMR (400 MHz, DMSO-d6) δ 13.1 (1H, br s); 8.21 (1H, br s); 7.59 (1H, d, J=8.3 Hz); 7.45 (1H, d, J=9.0 Hz); 7.35-7.20 (5H, m); 4.97 (2H, s); 4.17 (2H, t, J=7.2 Hz); 3.06 (2H, q, J=6.4 Hz); 2.51 (3H, s); 1.69 (2H, quintet, J=6.9 Hz). LCMS: Method B, Rₜ=4.00 min, [467/469 (ES−, M−H), 491/493 (ES+, MNa+)].

### EXAMPLE 44

### Preparation of [3-(3,8-Dimethyl-4-oxo-2,4-dihydro-pyrazolo[4,3-c]quinolin-5-yl)-propyl]-carbamic acid tert-butyl ester

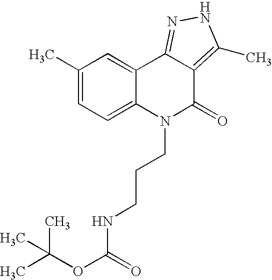

Using the appropriate reagents and in manner similar to that exemplified in Example 29 the title compound was obtained as a white solid. ¹H NMR (400 MHz, DMSO-d6) δ 7.92 (1H, m); 7.46-7.34 (2H, m); 6.88 (1H, t, J=5.2 Hz); 4.22 (2H, t, J=6.8 Hz); 3.06-2.99 (2H, dd, 6.76, 12.62 Hz); 2.56 (3H,s); 2.40 (3H, s); 1.76-1.69 (2H, m); 1.38 (9H, s). LCMS: Method A, Rₜ=2.91 min, m/z=371 (ES+, M+H), 369 (ES−, M−H).

### EXAMPLE 45

### Preparation of 5-(3-Amino-propyl)-3,8-dimethyl-2,5-dihydro-pyrazolo[4,3-c]quinolin-4-one

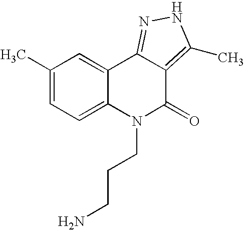

Boc deprotection of Example 44 in a manner similar to that exemplified in Example 11 affords the title compound as a white solid. ¹H NMR (400 MHz, DMSO) δ 7.99 (1H, d, J=0.73 Hz); 7.95 (2H, br,s); 7.56 (1H, d, J=8.78 Hz); 7.42 (1H, dd, J=8.59, 1.83 Hz); 4.33 (2H, t, J=6.94 Hz); 2.88 (2H, m); 2.42 (3H, s);

2.58 (3H, s); 1.96 (3H, t, 7.4 Hz). LCMS: Method B, Rₜ=2.54 Min, [ES−, MH−=269].

### EXAMPLE 46

### Preparation of Preparation of [3-(8-Bromo-3-methyl-4-oxo-1,4-dihydro-pyrazolo[4,3-c]quinolin-5-yl)-2-S-methyl-propyl]-carbamic acid tert-butyl ester

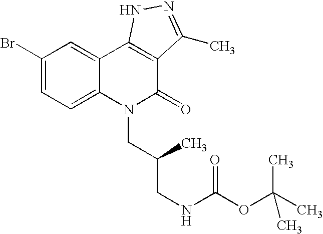

Using the appropriate reagent and in manner similar to that exemplified in Example 29 the title compound was obtained as a white solid.

¹H NMR (400 MHz, DMSO-d6) δ 13.78 (1H, s); 8.28 (1H, s); 7.67 (1H, d, J=8.6 Hz); 7.48 (1H, d, J=9.0 Hz); 6.84 (1H, m); 4.13 (2H, m); 2.99 - 2.92 (1H, m); 2.88-2.80 (1H, m); 2.58 (3H, s); 2.09 (1H, m); 1.36 (9H, s); 0.83 (3H, d, J=6.6 Hz).

LCMS: Method B, Rₜ=4.05 min, [MNa+=471/473].

### EXAMPLE 47

### Preparation of 5-(3-Amino-2-S-methyl-propyl)-8-bromo-3-methyl-1,5-dihydro-pyrazolo[4,3-c]quinolin-4-one

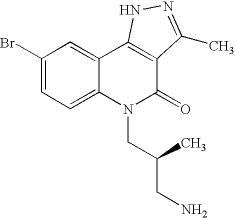

Boc deprotection of Example 46 in a manner similar to that exemplified in Example 11 affords the title compound as a white solid. ¹H NMR (400 MHz, DMSO) δ 8.34 (1H, d, J=2.2 Hz); 7.94 (3H, br s); 7.70 (1H, dd, J=9.0, 2.2 Hz); 7.59 (1H, d, J=9.2 Hz); 4.35-4.29 (1H, m); 4.13-4.09 (1H, m); 2.85-2.60 (4H, m); 2.58 (3H, s); 2.40-2.20 (1H, m); 1.01 (3H, d, J=6.5 Hz). LCMS: Method B, Rₜ=2.89 min, [MH+=349/].

### EXAMPLE 48

### Preparation of 8-Bromo-3-methyl-5-piperidin-3-ylmethyl-1,5-dihydro-pyrazolo [4,3-c]quinolin-4-one

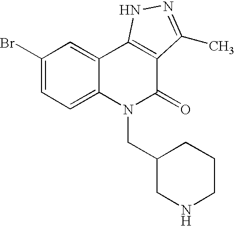

Boc deprotection of Example 30 in a manner similar to that exemplified in Example 11 afforded the title compound as a white solid. ¹H NMR (400 MHz, D₂O) δ 7.50 (1H, d, J=2.3 Hz); 7.39 (1H, dd, J=9.1, 2.1 Hz); 6.99 (1H, d, J=9.2 Hz); 4.04-3.98 (1H, m); 3.72-3.68 (1H, m); 3.22 (1H, m); 3.10 (1H, m); 2.84 (1H, td, J=12.5, 2.8 Hz); 2.75 (1H, t, J=12.0 Hz); 2.43 (3H, s); 2.12-2.00 (1H, m); 1.90-1.80 (1H, m); 1.80-1.70 (1H, m); 1.60-1.42 (1H, m); 1.41-1.28 (1H, m). LCMS: Method B, Rₜ=2.84 min, [MH+=375/377].

### EXAMPLE 49

### Preparation of 8-Chloro-3-(2-methoxy-ethyl)-2,5-dihydro-pyrazolo[4,3-c]quinolin-4-one

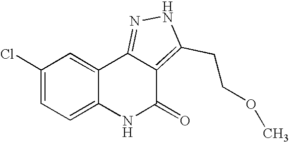

### Step 1: Preparation of 5-Chloro-2-(5-methoxy-3-oxo-pentanoylamino)-benzoic acid methyl ester

A solution of methyl 2-amino-5-chlorobenzoate (3.36 g, 18.1 mmol) and methyl-5-methoxy-3-oxovalerate (2.64 mL, 18.1 mmol) in toluene (20 mL) is heated at reflux for 40 h. After cooling the solvent is removed in vacuo and the residue purified by silica gel chromatography (20% then 50% EtOAc/isohexane) providing the desired keto-amide as an orange solid (3.40 g, 60% yield). ¹H NMR (400 MHz, DMSO, data for keto tautomer, which is >90% of the mixture in DMSO) δ 10.50 (1H, s); 8.05 (1H, d, J=9.0 Hz); 7.73 (1H, d, J=2.7 Hz); 7.56 (1H, dd, J8.9, 2.6 Hz); 3.73 (3H, s); 3.57 (2H, s); 3.44 (2H, t, J =6.2 Hz); 3.10 (3H, s); 2.67 (2H, t, J=6.3 Hz).

### Step 2: Preparation of 6-Chloro-4-hydroxy-3-(3-methoxy-propionyl)-1H-quinolin-2-one

A suspension of 5-chloro-2-(5-methoxy-3-oxo-pentanoylamino)-benzoic acid methyl ester (3.03 g, 9.7 mmol, 1 equiv.) in methanol is treated with sodium methoxide (1.05 g, 19.4 mmol, 2 equiv.) providing a solution which is heated at reflux for 1 h. 1M HCl_{(aq)} (19 mL) is added dropwise providing a slurry which is filtered. The pale yellow residual solid is washed with water 3 times, ether 3 times, and dried. The desired product is obtained as a pale yellow solid (2.46 g, 90% yield). ¹H NMR (400 MHz, DMSO) δ 7.89 (1H, d, J=2.4 Hz); 7.68 (1H, dd, J=8.9, 2.5 Hz); 7.28 (1H, d, J=9.0 Hz); 3.66 (2H, t, J=6.3 Hz); 3.43 (2H, t, J=6.2 Hz); 3.21 (3H, s).

### Step 3: Preparation of 8-Chloro-3-(2-methoxy-ethyl)-2,5-dihydro-pyrazolo[4,3-c]quinolin-4-one

A slurry of 6-chloro-4-hydroxy-3-(3-methoxy-propionyl)-1H-quinolin-2-one (1.0 g, 3.5 mmol) in DMF (14 mL) is treated with hydrazine hydrate (0.640 mL, 10.5 mmol, 3 equiv.) and the resultant yellow solution heated at 150° C. for 1 h. On cooling a precipitate forms which is taken up in ether, filtered and the solid washed twice with ether and dried. The desired product is obtained as a pale yellow powder (0.713 g, 73% yield). ¹H NMR (400 MHz, DMSO) δ 8.07 (1H, br,s); 7.50 (1H, d, J=9.0 Hz); 7.38 (1H, d, J=8.6 Hz); 3.72 (2H, t, J=6.9 Hz); 3.25 (3H, s); 3.21 (2H, m). LCMS: Method D, Rₜ=7.83 min. 278/280 (ES+, M+H), 300/302 (ES+, M+Na), 246/248 (ES+, M-MeOH), 276/278 (ES−, M−H).

### EXAMPLE 50

### Preparation of 5-(3-Amino-propyl)-8-chloro-3-(2-methoxy-ethyl)-2,5-dihydro-pyrazolo[4,3-c]quinolin-4-one

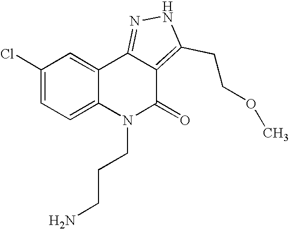

The title compound is prepared from Example 49 by the following steps.

### Step 1: Preparation of 8-Chloro-3-(2-methoxy-ethyl)-2-(tetrahydro-pyran-2-yl)-2,5-dihydro-pyrazolo[4,3-c]quinolin-4-one

A suspension of 8-chloro-3-(2-methoxy-ethyl)-2,5-dihydro-pyrazolo[4,3-c]quinolin-4-one (0.700 g, 2.52 mmol, 1 equiv.) in DMF (30 mL) is heated to 60° C. and treated with 3,4-dihydro-2H-pyran (0.915 mL, 10 mmol, 4 equiv.) and para-toluene sulfonic acid (0.048 g, 0.25 mmol, 0.1 equiv.) and stirring continued. After 21 h, a further 4 equivalents of 3,4-dihydro-2H-pyran and 0.1 equivalents of para-toluene sulfonic acid are added and the suspension heated to 70° C. After a further 72 h the suspension is filtered and the solid residue washed with ether three times and dried. The desired tetrahydro-pyranyl pyrazine is obtained as a white solid (0.520 g, 57% yield).

¹H NMR (400 MHz, DMSO) δ 11.19 (1H, s); 7.94 (1H, d, J=2.4 Hz); 7.48 (1H, dd, J=8.7, 2.5 Hz); 7.34 (1H, d, J=8.8 Hz); 5.74 (1H, dd, J=9.5, 2.4 Hz); 3.95 (1H, m); 3.74 (1H, m); 3.62 (2H, m); 3.43 (2H, m); 3.25 (3H, s); 2.43 (1H, m); 2.06 (1H, m); 1.93 (1H, m); 1.75 (1H, m); 1.60 (2H, m). LCMS: Method A, Rₜ=3.34 min. m/z=362/364 (ES+, M+H), 278/280 (ES+, M-tetrahydropyran).

### Step 2: Preparation of {3-[8-Chloro-3-(2-methoxy-ethyl)-4-oxo-2-(tetrahydro-pyran-2-yl)-2,4-dihydro-yrazolo[4,3-c]quinolin-5-yl]-propyl}-carbamic acid tert-butyl ester

A suspension of 8-chloro-3-(2-methoxy-ethyl)-2-(tetrahydro-pyran-2-yl)-2,5-dihydro-pyrazolo[4,3-c]quinolin-4-one (0.180 g, 0.5 mmol, 1 equiv.) in DMF (7 mL) is treated with potassium tert-butoxide (0.056 g, 0.5 mmol, 1 equiv.) and stirred for 10 min before addition of potassium carbonate (0.276 g, 2 mmol, 4 equiv.) and a solution (3-bromo-propyl)-carbamic acid tert-butyl ester (0.298 g, 1.25 mmol, 2.5 equiv.) in DMF (3 mL). The suspension is heated at 90° C. for 16 h. The cooled reaction mixture is partitioned between water and DCM and the aqueous phase separated and extracted with DCM 3 times. The combined organic phases are washed with water 4 times, then brine, dried over Na₂SO₄, filtered and concentrated. The crude residue is purified by silica gel chromatography (50% EtOAc/isohexane) to provide the desired product as a white solid (0.137 g, 53% yield). ¹H NMR (CDCl₃, 400 MHz) δ 8.31 (1H, d, J=2.5 Hz); 7.43 (1H, dd, J=8.9, 2.5 Hz); 7.25 (1H, d, J=9.3 Hz); 5.70 (1H, dd, J=9.5, 2.6 Hz); 5.47 (1H, br s); 4.34 (2H, m); 4.12 (1H, m); 3.82-3.60 (4H, m); 3.42 (1H, m); 3.32 (3H, s); 3.14 (2H, m); 2.62 (1H, m); 2.20 (1H, m); 1.98 (1H, m); 1.92 (2H, quintet, J=6.4 Hz); 1.79 (2H, m); 1.65 (1H, m); 1.46 (9H, s). LCMS, Method A, Rₜ=4.11 minutes, >95% pure. m/z=519/521 (ES+, M+H), 541/543 (ES+, M+Na).

### Step 3: Preparation of 5-(3-Amino-propyl)-8-chloro-3-(2-methoxy-ethyl)-2,5-dihydro-pyrazolo[4,3-c]quinolin-4-one

{3-[8-chloro-3-(2-methoxy-ethyl)-4-oxo-2-(tetrahydro-pyran-2-yl)-2,4-dihydro-pyrazolo[4,3-c]quinolin-5-yl]-propyl}-carbamic acid tert-butyl ester (0.06 g, 0.116 mmol) is dissolved in 1.25 M HCl/MeOH (10 mL) and the solution stirred for 2 h. Where incomplete deprotection is observed the solvent is removed in vacuo and the residue dissolved in 1.25M HCl/MeOH (10 mL) and stirred for a further 2 h. The solvent is removed in vacuo and the residue concentrated from methanol 3 times before drying. The hydrochloride salt is obtained as a white solid (46 mg, quantitative yield based on 2HCl). ¹H NMR (400 MHz, D₂O) δ 7.43 (1H, br s); 7.33 (1H, d, J=9.1 Hz); 7.22 (1H, d, J=9.2 Hz); 4.17 (2H, t, J=6.6 Hz); 3.90 (2H, t, J=6.4 Hz); 3.44 (3H, s); 3.29 (2H, t, J=6.3 Hz); 3.08 (2H, t, J=7.3 Hz); 2.07 (2H, quintet, J=7.1 Hz). LCMS, Method B, Rₜ=2.73 min. m/z=333/335 (ES−, M−H), 335/337 (ES+, M+H), 357/359 (ES+, M+Na).

### EXAMPLE 51

### Preparation of 3-Methyl-5-(3-phenethylamino-propyl)-1,5-dihydro-pyrazolo[4,3-c]quinolin4-one

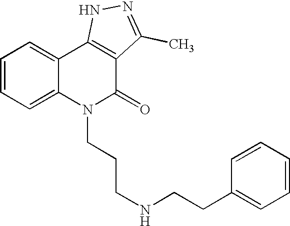

Using the appropriate reagent and in a similar manner as for Example 29, and hydrolysis of intermediate Boc protected derivative as in Example 15, the title compound was obtained as a white solid. ¹H NMR (400 MHz, DMSO-d6) δ 9.06 (1H, br s); 8.18 (1H, dd, J=8.0, 1.4 Hz); 7.66 (1H, d, J=8.4 Hz); 7.59 (1H, td, J=7.8, 1.2 Hz); 7.4-7.2 (6H, m); 4.36 (2H, t, J=7.0 Hz); 3.14 (2H, m); 3.04 (2H, m); 2.95 (2H, m); 2.58 (3H, s); 2.06 (2H, quintet, J=7.2 Hz). LCMS: Method B, Rₜ=3.30 Min, [MH+=361].

### EXAMPLE 52

### Preparation of [2-(8-Bromo-3-methyl-4-oxo-1,4-dihydro-pyrazolo[4,3-c]quinolin-5-yl)-ethyl]-carbamic acid tert-butyl ester

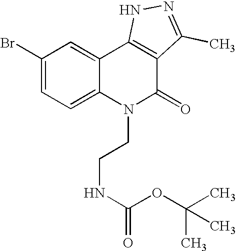

Using the appropriate reagent and in manner similar to that exemplified in Example 29 the title compound was obtained as a white solid. ¹H NMR (400 MHz, DMSO-d6) δ 8.2 (1H, br s); 7.64 (1H, d, J=8.4 Hz); 7.57 (1H, d, J=8.8 Hz); 6.96 (1H, t, J=5.8 Hz); 4.18 (2H, t, J=6.2 Hz); 3.12 (2J, q, J=6.2 Hz); 2.51 (3H, s); 1.25 (9H s). LCMS: Method B, Rₜ=3.64 min, [MH+=421/423].

### EXAMPLE 53

### Preparation of 5-(2-Amino-ethyl)-8-bromo-3-methyl-1,5-dihydro-pyrazolo[4,3-c]quinolin4-one

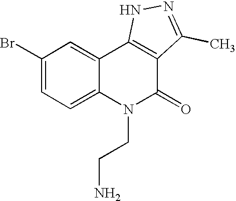

Boc deprotection of Example 52 in a manner similar to that exemplified in Example 11 affords the title compound as a white solid. ¹H NMR (400 MHz, D₂O) δ 7.61 (1H, d, J=2.4 Hz); 7.51 (1H, dd, J=8.8, 2.4 Hz); 7.09 (1H, d, J=9.2 Hz); 4.33 (2H, t, J=6.0 Hz); 3.25 (2H, t, J=6.0 Hz); 2.49 (3H, s). LCMS: Method B, Rₜ=2.98 min, [MH+=321/323].

### EXAMPLE 55

### Preparation of 5-(3-Amino-propyl)-3-methyl-8-nitro-2,5-dihydro-pyrazolo[4,3-c]quinolin-4-one (route 2)

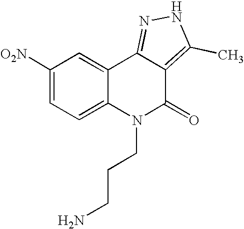

### Step 1: Preparation of 5-Nitro-2-(3-oxo-butyrylamino)-benzoic acid methyl ester

To a suspension of 4-nitro methyl anthranilate (25.6 mmol, 5 g) in toluene (60 ml) was added 2,2,6-trimethyl-1,3-dioxin-4-one (28.2 mmol, 3.78 ml). The solution was refluxed for 16 hours and left to stand at RT for 1 hour, the formed precipitate was successively filtered, washed with toluene and dried to afford 5-Nitro-2-(3-oxo-butyrylamino)-benzoic acid methyl ester as a yellow solid (5.61g, 78%). ¹H NMR (400 MHz, DMSO-d₆) δ appear as mixture of tautomers, enol form not described, 11.06(s, 1H, broad); 8.66(d, 1H, J=2.74); 8.50(d, 1H, J=9.3); 8.46(dd, 1H, J=9.3, 2.7); 3.93(s, 3H); 3.802(s,˜2H); 2.24(s, 3H).

### Step 2: Preparation of 3-Acetyl-6-nitro-1H-quinoline-2,4-dione

To a suspension of 5-Nitro-2-(3-oxo-butyrylamino)-benzoic acid methyl ester (16.2 mmol, 4.55 g) in MeOH (390 ml) was added sodium methoxide (65 mmol, 3.51 g), the reaction mixture was refluxed for 6 hours and left to stand overnight. The obtained slurry was concentrated under vacuum and the residue suspended in H₂O (160 ml). The mixture was acidified to pH 2 with 4M HCl_{(aq)} (16 ml), the formed precipitate was successively filtered, washed with H₂O (30 ml), Et₂O (2×100 ml) and dried to afford 3-Acetyl-6-nitro-1H-quinoline-2,4-dione as a white/beige solid (3.83g, 95%). ¹H NMR (400 MHz, DMSO-d₆) δ appear as mixture of tautomers, enol form, 11.94 (s, 0.6H, broad); 8.49 (d, 1H, J=2.56); 8.26 (dd, 1H, J=9.1, 2.6); 7.23 (d, 1H, J=9.1); 2.53 (s, 3H).

### Step 3: Preparation of 3-Methyl-8-nitro-2,5-dihydro-pyrazolo[4,3-c]quinolin-4-one

To a suspension of 3-Acetyl-6-nitro-1H-quinoline-2,4-dione (15.44 mmol, 3.83 g) in DMF (90 ml) was added hydrazine hydrate (46.4 mmoles, 1.45 ml). The resulting solution was refluxed for 6 hours. Upon completion the reaction mixture was left to cool to 60° C. and the formed precipitate was successively filtered, washed with MeOH (2×15 ml), Et₂O (2×60 ml) and dried to afford 3-Methyl-8-nitro-2,5-dihydro-pyrazolo[4,3-c]quinolin-4-one as a white solid (3.1 g, 83%). ¹H NMR (400 MHz, DMSO-d6) δ 13.8 (s, 1H, broad); 11.8 (s, 1H, broad); 8.91-8.88 (s, 1H, broad); 8.30 (dd, 1H, J=9.1, 2.6); 7.48 (d, 1H, J=9.1); 2.58 (s, 3H).

### Step 4: Preparation of 3-Methyl-8-nitro-2-(tetrahydro-pyran-2-yl)-2,5-dihydro-pyrazolo[4,3-c]quinolin-4-one

To a suspension of 3-methyl-8-nitro-2,5-dihydro-pyrazolo[4,3-c]quinolin-4-one (12.7 mmoles, 1 eq, 3.1 g) in DMF (200 ml) was successively added 3,4-dihydro-2H-pyran (50.8 mmoles, 4 eq, 4.6 g) and para-toluenesulfonic acid (1.2 mmoles, 0.1 eq, 228 mg). The mixture was heated at 90° C. for 2 days. The reaction mixture was concentrated under vacuum and the residue retreated with the above reagents and under the same conditions for 4 hours. The obtained solution was left to stand over the weekend and the formed precipitate was filtered and washed with MeOH/Et₂O to afford 3-Methyl-8-nitro-2-(tetrahydro-pyran-2-yl)-2,5-dihydro-pyrazolo[4,3-c]quinolin-4-one as a white solid (1.74 g, 42%). ¹H NMR (400 MHz, DMSO-D₆) δ 8.73 (d, 1H, J=2.56), 8.29 (dd, 1H, J=8.9, 2.3); 7.46 (d, 1H, J=9.1); 5.71 (dd, 1H, J=9.5, 2.37); 3.98-3.92 (m, 1H); 3.78-3.72 (m, 1H); 2.74 (s, 3H); 2.48-2.42 (m, 1H); 2.11-1.98 (m, 2H); 1.81-1.68 (m, 1H); 1.68-1.58 (m, 2H).

### Step 5: Preparation of {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester

To a suspension of 3-Methyl-8-nitro-2-(tetrahydro-pyran-2-yl)-2,5-dihydro-pyrazolo[4,3-c]quinolin-4-one (1 mmol, 328 mg) in DMF(10 ml) at RT was added t-BuOK (1 mmol, 112 mg), The mixture was stirred for 5 minutes then K₂CO₃ (4 mmol, 552 mg) was added followed by a solution of the alkyl halide (2.5 mmol, 593 mg) in DMF (10 ml). The mixture was heated at 90° C. for 3 hours, then concentrated under vacuum, the residue was partitioned between H₂O (160 ml) and DCM (150 ml). The aqueous layer was decanted and extracted with DCM (3×150 ml), then combined chlorinated layers were concentrated under vacuum to afford a crude mixture of N-alylated compound and O-alkylated derivative. Flash column separation and purification (70/30 Isohexane/EtOAc: 50/50) afford the title compound {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester as a white solid (300 mg, 62%). ¹H NMR (400 MHz, CDCl₃) δ 9.09 (d, 1H, J=2.6); 8.25 (dd, 1H, J=9.3, 2.7); 7.34 (d, 1H, 9.51); 5.47 (dd, 1H, J=9.51, 2.7); 5.25 (d, broad, 1H); 4.31 (t, 2H, J=6.6); 3.69-3.63 (m, 1H); 4.04-4.00 (m, 1H); 3.12-3.07 (m, 2H); 2.78 (s, 3H); 2.61-2.52 (m, 1H); 2.19-2.13 (m, 1H); 2.00-1.95 (m, 1H); 1.90-1.83 (m, 2H); 1.77-1.56 (m, 3H); 1.39 (s, 9H).

### Step 6: Preparation of 5-(3-Amino-propyl)-3-methyl-8-nitro-2,5-dihydro-pyrazolo[4,3-c]quinolin-4-one

Similar treatment of {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester with aqueous TFA as in Example 16, affords the title compound as a white solid. ¹H NMR (400 MHz, DMSO-d6) δ 9.09(s, 1H, broad); 8.44 (dd, 1H, J=9.1, 2.6 Hz); 7.95 (dₐₚₚₐᵣₑₙₜ, 3H, NH2+H, J=9.1 Hz, broad); 4.47 (t, 2H, J=6.9 Hz); 3.02-2.95 (m, 2H); 2.69 (s, 3H); 2.09-2.02 (m, 2H). LCMS condition D, 20-100%, Rₜ=6.3 min [MH+=302/303].

### EXAMPLE 56

### Preparation of [3-(8-Bromo-3-methyl-4-oxo-1,4-dihydro-pyrazolo[4,3-c]quinolin-5-yl)-propyl]-carbamic acid tert-butyl ester

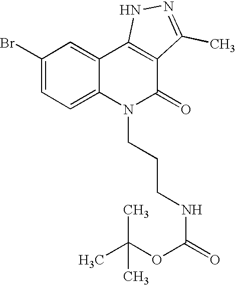

Using the appropriate reagent and in manner similar to that exemplified in Example 29 the title compound was obtained as a white solid. ¹H NMR (400 MHz, DMSO-d6) δ 13.76 (1H, br s); 8.27 (1H, br s); 7.68 (1H, d, J=8.3 Hz); 7.51 (1H, d, J=8.8 Hz); 6.90 (1H, t, J=5.5 Hz); 4.21 (2H, t, J=7.0 Hz); 3.02 (2H, q, J=6.4 Hz); 2.57 (3H, br s); 1.72 (2H, quintet, J=7.1 Hz); 1.38 (9H, s). LCMS: Method B, Rₜ=3.79 min, [MH+=435/437].

### EXAMPLE 57

### Preparation of 5-(3-Amino-propyl)-8-bromo-3-methyl-1,5-dihydro-pyrazolo[4,3-c]quinolin-4-one

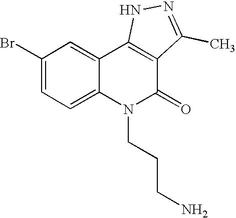

Boc deprotection of Example 56 in a manner similar to that exemplified in Example 11 affords the title compound as a white solid. ¹H NMR (400 MHz, D₂O) δ 7.30-7.25 (2H, m); 6.93 (1H, d, J=9.7 Hz); 3.92 (2H, t, J=6.8 Hz); 2.90 (2H, t, J=7.4 Hz); 2.36 (3H, s); 1.88 (2H, quintet, J=7.3 Hz). LCMS: Method B, Rₜ=2.95 min, [MH+=335/337].

### EXAMPLE 58

### Preparation of 5-(3-Amino-propyl)-8-methoxy-3-methyl-2,5,5a,9a-tetrahydro-pyrazolo[4,3-c]quinolin-4-one

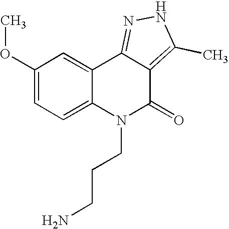

Boc deprotection of Example 79 in a manner similar to that exemplified in Example 11 affords the title compound as a white solid. ¹H NMR (400 MHz, DMSO-d₆) δ 7.91 (2H, br,s); 7.75 (1H, d, J=2.92 Hz); 7.58 (1H, d, J=9.33 Hz); 7.18 (1H, dd, J=2.92, 9.33 Hz); 4.32 (2H, t, 6.76 Hz); 3.86 (3H, s); 2.86 (2H, m); 2.57 (3H, s); 1.95 (3H, t, 7.2 Hz). LCMS: Method A, Rₜ=1.55 min, [MH+=287].

### EXAMPLE 59

### Preparation of 5-(3-Amino-propyl)-8-hydroxy-3-methyl-2,5-dihydro-pyrazolo[4,3-c]quinolin-4-one

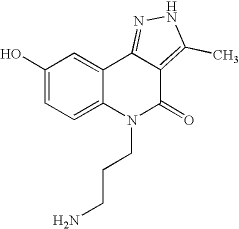

5-(3-Amino-propyl)-8-methoxy-3-methyl-2,5-dihydro-pyrazolo[4,3-c]quinolin-4-one hydrochloride salt (0.070 g, 0.25 mmol) was suspended in dichloromethane (4 ml), cooled at 0° C. and treated dropwise with BBr₃ (1M in DCM, 5 ml) under a nitrogen atmosphere. The reaction was stirred for 3 days, quenched with methanol and evaporated to dryness. The residue was dissolved in ethyl acetate (5 ml) and washed with sat NaHCO_{3 (aq)} (5 ml) and water (3 ml). The aqueous layers were concentrated and the residue re-dissolved in ethyl acetate. After filtration the solution was dried over sodium sulfate, filtered and concentrated to give an off white solid (0.025 g, 37%), contaminated by 7% of starting material. ¹H NMR (400 MHz, DMSO-d₆) δ 8.03 ppm (2H, br, s); 7.46 (1H, d, J=2.74 Hz); 7.41 (1H, d, 9.33 Hz); 7.0 (1H, dd, J=2.74, 9.14 Hz); 4.22 (2H, t, J=6.76 Hz); 2.80-2.75 (2H, m); 2.50 (3H, s); 1.93-1.85 (2H, m). LCMS: Method B, Rₜ=2.05 min, MH+=273; MH−=271.

### EXAMPLE 60

### Preparation of 4-Chloro-N-[3-(3-methyl-4-oxo-2,4-dihydro-pyrazolo[4,3-c]quinolin-5-yl)-propyl]-benzamide

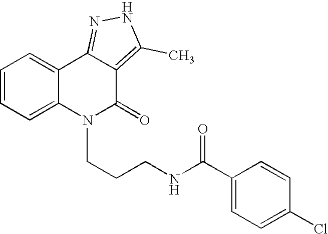

Using the appropriate reagent and in manner similar to that exemplified in Example 35 the title compound was obtained as a white solid. ¹H NMR (400 MHz, DMSO-d6) δ 8.66 (1H, t, J=5.67 Hz); 8.11 (1H, dd, J=0.54, 7.7 Hz); 7.87 (2H, d, J=8.41 Hz); 7.58-7.53 (4H, m); 7.32-7.28 (1H, m); 4.32 (2H, t, J=7.13 Hz); 3.41-3.36 (2H, m); 2.57 (3H, s); 1.93-1.86 (2H, m). LCMS, Method D, 40-100% B, Rₜ=9.50 min, m/z=395.3 (ES+, M+H), 417.28 (ES+, M+Na).

### EXAMPLE 61

### Preparation of N-[3-(3-Methyl-4-oxo-2,4-dihydro-pyrazolo[4,3-c]quinolin-5-yl)-propyl]-4-nitro-benzamide

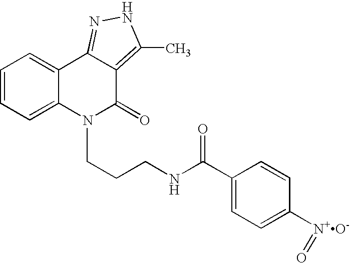

Using the appropriate reagent and in manner similar to that exemplified in Example 35 the title compound was obtained as a white solid. ¹H NMR (400 MHz, DMSO-d6) δ 8.87 (1H, t, J=5.48 Hz); 8.35-8.31 (2H, m); 8.12 (1H, dd, J=0.73, 7.31 Hz); 8.09-8.06 (2H, m); 7.60-7.52 (2H, m); 7.33-7.28 (1H, m); 4.35 (2H, t, J=6.95 Hz); 3.45-3.40 (2H, m); 2.57 (3H, s); 1.96-1.89 (2H, m). LCMS: Method C, 20-100%, Rₜ=6.62 min, MH+=406.23; MH−=404.22.

### EXAMPLE 62

### Preparation of N-[3-(3-Methyl-4-oxo-2,4-dihydro-pyrazolo[4,3-c]quinolin-5-yl)-propyl]-3-phenyl-propionamide

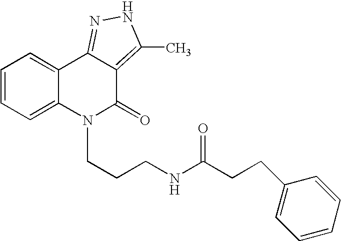

Using the appropriate reagents and in manner similar to that exemplified in Example 35 the title compound was obtained as a white solid. ¹H NMR (400 MHz, DMSO-d6) δ 7.87 (1H, dd, J=7.8, 1.4 Hz); 7.68 (1H, t, J=5.5 Hz); 7.36-7.24 (2H, m); 7.06 (1H, t, J=7.0 Hz); 7.04-6.92 (4H, m); 6.90 (1H, t, J=7.6 Hz); 3.98 (2H, t, J=7.3 Hz); 2.91 (2H, q, J=6.5 Hz); 2.57 (2H, t, J=7.8 Hz); 2.33 (3H, br s); 2.14 (2H, t, J=7.8 Hz); 1.48 (2H, quintet, J=7.2 Hz). LCMS: Method D, 40-100%, Rₜ=8.86 min, MH+=389.38; MNa+=411.34.

### EXAMPLE 63

### Preparation of 2,4-Difluoro-N-[3-(3-methyl-4-oxo-2,4-dihydro-pyrazolo[4,3-c]quinolin-5-yl)-propyl]-benzamide

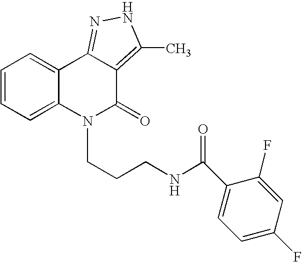

Using the appropriate reagents and in manner similar to that exemplified in Example 35 the title compound was obtained as a white solid. ¹H NMR (400 MHz, DMSO-d6) δ 13.76 (1H, br s); 8.50 (1H, m); 8.16 (1H, d, J=7.4 Hz); 7.76 (1H, m); 7.61 (2H, m); 7.41 (1H, m); 7.35 (1H, t, J=6.6 Hz); 7.22 (1H, td, J=5.6, 2.4 Hz); 4.36 (2H, t, J=7.1 Hz); 3.40 (2H, m); 2.61 (3H, s); 1.92 (2H, quintet, J=7.1 Hz). LCMS: Method D, 40-100% B, Rₜ=9.01 min, 397.32 (ES+, M+H), 419.31 (ES+, M+Na).

### EXAMPLE 64

### Preparation of 4-Fluoro-N-[3-(3-methyl-4-oxo-2,4-dihydro-pyrazolo[4,3-c]quinolin-5-yl)-propyl]-benzamide

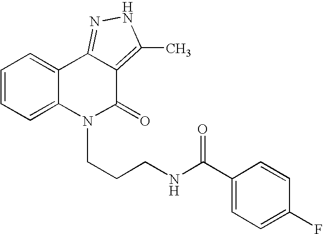

Using the appropriate reagents and in manner similar to that exemplified in Example 35 the title compound was obtained as a white solid. ¹H NMR (400 MHz, DMSO-d6) δ 13.70 (1H, br s); 8.56 (1H, t, J=5.6 Hz); 8.11 (1H, d, J=7.5 Hz); 7.91 (2H, dd, J=8.8 5.7 Hz); 7.56 (2H, m); 7.30 (3H, s); 4.32 (2H, t, J=7.3 Hz); 3.38 (2H, q, J=6.5 Hz); 2.57 (3H, s); 1.89 (2H, quintet, J=7.1 Hz). LCMS, Method C, 20-100% B, Rₜ=6.45 min, 379.23 (ES+, M+H).

### EXAMPLE 65

### Preparation of Cyclohexanecarboxylic acid [3-(3-methyl-4-oxo-2,4-dihydro-pyrazolo[4,3-c]quinolin-5-yl)-propyl]-amide

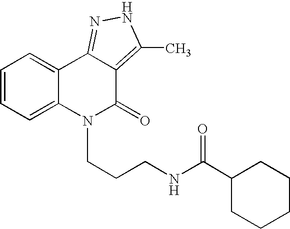

Using the appropriate reagents and in manner similar to that exemplified in Example 35 the title compound was obtained as a white solid. ¹H NMR (400 MHz, DMSO-d6) δ 8.12 (1H, dd, J=7.6, 1.3 Hz); 7.76 (1H, t, J=5.1 Hz); 7.54 (2H, m); 7.30 (1H, t, J=7.5 Hz); 4.24 (2H, t, J=7.5 Hz); 3.14 (2H, quartet, J=6.5); 2.05-2.12 (1H, m); 1.6-1.8 (6H, m); 1.25-1.40 (3H, m), 1.15-1.25 (3H, m). LCMS, Method D, 40-100%, Rₜ=9.62 min, 367.35 (ES+M+H); 365.38 (ES−M−H).

### EXAMPLE 66

### Preparation of 1-[3-(3-Methyl-4-oxo-1,4-dihydro-pyrazolo[4,3-c]quinolin-5-yl)-propyl]-3-phenyl-urea

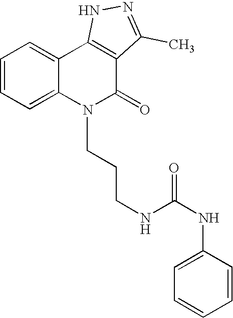

The title compound was prepared from Example 15 by the following procedure. A solution of 5-(3-amino-propyl)-3-methyl-1,5-dihydro-pyrazolo[4,3-c]quinolin-4-one (Example 15) (0.058 g, 0.2 mmol) in DMF (1 mL) and diisopropylethylamine (0.070 mL, 0.4 mmol) is treated with phenyl isocyanate (0.028 mL, 0.25 mmol) and stirred at room temperature. After 2 h the solution is diluted with ethyl acetate, washed with water, dried over Na₂SO₄, filtered and concentrated. The crude white solid is purified by silica gel chromatography (50%-80%-100% EtOAc/hexane) providing 0.040 g (53% yield) of a white powder. ¹H NMR (400 MHz, DMSO-d6) δ 8.12 (1H, dd, J=7.6, 1.3 Hz); 7.76 (1H, t, J=5.1 Hz); 7.54 (2H, m); 7.30 (1H, t, J=7.5 Hz); 4.24 (2H, t, J=7.5 Hz); 3.14 (2H, quartet, J=6.5); 2.05-2.12 (1H, m); 1.6-1.8 (6H, m); 1.25-1.40 (3H, m), 1.15-1.25 (3H, m). LCMS: Method B, Rₜ=3.37 min, [MH+=376].

### EXAMPLE 67

### Preparation of 1-[4-(8-Bromo-3-methyl-4-oxo-1,4-dihydro-pyrazolo[4,3-c]quinolin-5-yl)-butyl]-3-phenyl-urea

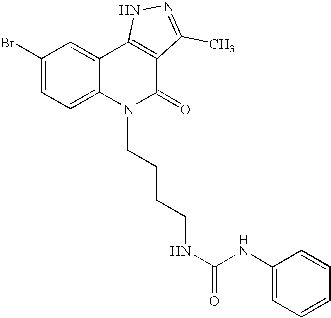

Using the appropriate reagents and in manner similar to that exemplified in Example 66 the title compound was obtained as a white solid. ¹H NMR (400 MHz, DMSO-d6) δ 8.41 (1H, s); 8.4-8.2 (1H, br s); 7.65 (1H, m); 7.57 (1H, m); 7.37 (2H, d, J=7.7 Hz); 7.20 (2H, t, J=7.9 Hz); 6.88 (1H, t, J=7.3 Hz); 6.15 (1H, t, J=5.7 Hz); 4.23 (2H, m); 3.14 (2H, q, J=6.2 Hz); 2.57 (3H, s); 1.66-1.49 (4H, m). LCMS: Method B, Rₜ=3.65 min, [MH+=468/470].

### EXAMPLE 68

### Preparation of 4-Cyano-N-[3-(3-methyl4-oxo-2,4-dihydro-pyrazolo[4,3-c]quinolin-5-yl)-propyl]-benzamide

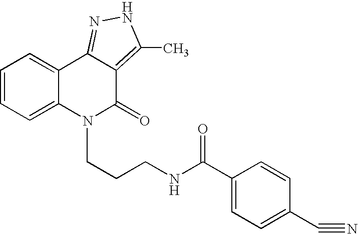

Using the appropriate reagents and in manner similar to that exemplified in Example 35 the title compound was obtained as a white solid. ¹H NMR (400 MHz, DMSO-d6) δ 13.80 (1H, br s); 8.92 (1H, t, J=5.6 Hz); 8;25 (1H, d, J=7.5 Hz); 8.13 (1H, d, J=8.6 Hz); 8.09 (2H, d, J=8.2 Hz); 7.70 (1H, m); 7.43 (1H, m); 4.46 (2H, t, J=7.2 HZ); 3.53 (2H, quintet, J=6.5 Hz); 2.70 (3H, s); 2.04 (2H, quintet, J=7.2 Hz). LCMS: Method D, 40-100% B, Rₜ=8.26 Min, [MH+=386.29].

### EXAMPLE 69

### Preparation of 4-Methoxy-N-[3-(3-methyl-4-oxo-2,4,5a,9a-tetrahydro-pyrazolo[4,3-c]quinolin-5-yl)-propyl]-benzamide

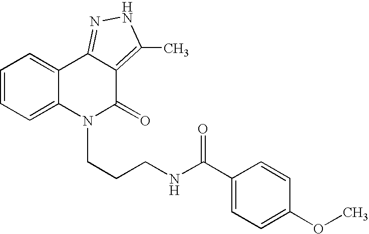

Using the appropriate reagents and in manner similar to that exemplified in Example 35 the title compound was obtained as a white solid. ¹H NMR (400 MHz, DMSO-d6) δ: 8.33 (1H, t, J=5.85 Hz); 8.02 (1H, dd, J=0.91, 7.5 Hz); 7.73 (2H, d, J=8.59 Hz); 7.49-7.41 (2H, m); 7.23-7.18 (1H, m); 6.90 (2H, d, J=8.59 Hz); 4.22 (2H, t, J=7.13 Hz); 3.71 (3H, s); 3.29-3.25 (2H, m); 2.48 (3H, s, br); 1.82-1.75 (2H, m). LCMS: Method D, 40-100%, RT=8.31 min, MH+=391.35; MH−=389.34.

### EXAMPLE 70

### Preparation of 5-(3-Amino-propyl)-7-chloro-3-methyl-2,5-dihydro-pyrazolo[4,3-c]quinolin-4-one

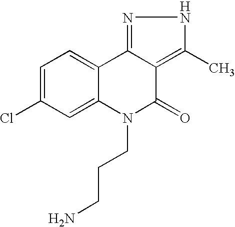

### Step 1: Preparation of 4-Chloro-2-(3-oxo-butyrylamino)-benzoic acid methyl ester

From methyl-2-amino-4-chlorobenzoate and using a similar manner as for Example 55, the title compound was obtained as a pale yellow solid (1.14 g, 80%). ¹H NMR (400 MHz, DMSO-d6) δ 10.75 (s, 1H); 8.32 (d, 1H, J=2.01 Hz); 7.8(d, 1H, J=8.59 Hz); 7.2(dd, 1H, J=2.19, 8.59 Hz); 3.80 (s, 3H); 3.66 (s, 2H); 2.16 (s, 3H).

### Step 2: Preparation of 3-Acetyl-7-chloro-1H-quinoline-2,4-dione

The title compound was obtained from 4-Chloro-2-(3-oxo-butyrylamino)-benzoic acid methyl ester using a similar cyclisation as for Example 55 (0.5 g, 50%). ¹H NMR (400 MHz, DMSO-d6) δ: appear as mixture of enol form, 7.86(d, 1H, J=8.59 Hz); 7.18 (d, 1H, J=1.83 Hz); 7.13 (dd, 1H, J=2.01, 8.59); 2.56 (s, 3H).

### Step 3: Preparation of 7-Chloro-3-methyl-1,5-dihydro-pyrazolo[4,3-c]quinolin-4-one

The title compound was obtained from 3-Acetyl-7-chloro-1H-quinoline-2,4-dione and using a similar condensation as for Example 55, as a white solid (86%). ¹H NMR (400 MHz, DMSO-d6) δ 13.69 (s, 1H, broad); 11.15 (s, 1H, broad); 8.0 (d, 1H, J=8.41 Hz); 7.38 (s, 1H); 7.26 (d, 1H, J=8.23 Hz); 2.56 (s, 3H).

### Step 4: Preparation of 7-Chloro-3-methyl-2-(tetrahydro-pyran-2-yl)-2,5-dihydro-pyrazolo[4,3-c]quinolin-4-one

From 7-Chloro-3-methyl-1,5-dihydro-pyrazolo[4,3-c]quinolin-4-one using a similar protection as for Example 55, the title compound was obtained as a white solid (56%). ¹H NMR (400 MHz, DMSO-d6) δ 11.23 (s, 1H, broad); 7.98 (d, 1H, J=8.4); 7.34 (d, 1H, J=2.01); 7.21 (dd, 1H, J=8.4, 2.1); 5.66 (dd, 1H, J=9.5, 2.1); 3.96-3.92 (m, 1H); 3.75-3.69 (m, 1H); 2.72 (s, 3H); 2.45-2.35 (m, 1H); 2.08-1.94 (m, 2H); 1.79-1.65 (m, 1H); 1.62-1.57 (m, 2H).

### Step 5: Preparation of {3-[7-Chloro-3-methyl-4-oxo-2-(tetrahydro-pyran-2-yl)-2,4-dihydro-pyrazolo[4,3-c]quinolin-5-yl]-propyl}-carbamic acid tert-butyl ester

From 7-Chloro-3-methyl-2-(tetrahydro-pyran-2-yl)-2,5-dihydro-pyrazolo[4,3-c]quinolin-4-one, using a similar alkylation as in Example 55, the title compound was obtained as white solid (59%). ¹H NMR (400 MHz, CDCl₃) δ 8.14 (d, 1H, J=8.2); 7.23 (d, 1H, J=1.6); 7.14 (dd, 1H J=8.2, 1.8); 5.45 (dd, 1H, J=9.7, 2.7); 5.42-5.38 (s,1H, broad); 4.24 (t, 2H, J=6.6); 4.06-4.00 (m, 1H); 3.68-3.61 (m, 1H); 3.10-3.04 (m, 2H); 2.76 (s, 3H); 2.58-2.48 (m, 1H); 2.15-2.10 (m, 1H); 1.97-1.92 (m, 1H); 1.88-1.81 (m, 2H); 1.76-1.56 (m, 3H); 1.38 (s, 9H).

### Step 6

From 7-Chloro-3-methyl-2-(tetrahydro-pyran-2-yl)-2,5-dihydro-pyrazolo[4,3-c]quinolin-4-one, using a similar deprotection as in Example 55, the title compound 5-(3-Amino-propyl)-7-chloro-3-methyl-2,5-dihydro-pyrazolo[4,3-c]quinolin-4-one was obtained as white solid (90%). ¹H NMR (400 MHz, DMSO-d6) δ 8.16 (d, 1H, J=8.4); 7.89 (s, 3H, broad); 7.72 (d, 1H, J=1.8); 7.39 (dd, 1H, J=8.4, 1.8); 4.33 (t, 2H, J=7.1); 2.92-2.79 (, 2H); 2.58 (s, 3H); 1.97-1.89 (m, 2H). LCMS, Method D, 20-100%, Rₜ=7.22 min [MH+=291].

### EXAMPLE 71

### Preparation of 3-Chloro-N-[3-(3-methyl-4-oxo-2,4-dihydro-pyrazolo[4,3-c]quinolin-5-yl)-propyl]-benzamide

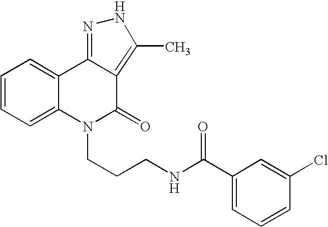

Using the appropriate reagent and in manner similar to that exemplified in Example 35 the title compound was obtained as a white solid. ¹H NMR (400 MHz, DMSO-d6) δ 8.70 (1H, t, J=5.7 Hz); 8.12 (1H, dd, J=7.7, 1.2 Hz); 7.89 (1H, t, J=1.9 Hz); 7.81 (1H, dm, J=7.7); 7.61 (1H, dm, J=7.9 Hz); 7.49-7.59 (3H, m); 7.30 (1H, t, J=7.2 Hz); 4.32 (2H, t, J=7.3 Hz); 3.38 (2H, quartet, J=6.5 Hz); 2.57 (3H, s); 1.92 (2H, quintet, J=7.2). LCMS: Method C, 20-100% B, Rₜ=7.11 min, [MH+=395.28].

### EXAMPLE 72

### Preparation of N-[3-(3-Methyl-4-oxo-2,4-dihydro-pyrazolo[4,3-c]quinolin-5-yl)-propyl]-4-trifluoromethyl-benzamide

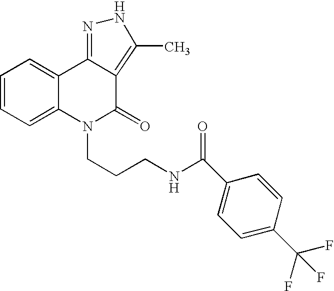

Using the appropriate reagents and in manner similar to that exemplified in Example 35 the title compound was obtained as a white solid. ¹H NMR (400 MHz, DMSO-d6) δ 8.80 (1H, t, J=5.67 Hz); 8.14 (1H, dd, J=1.09-7.68 Hz); 8.07 (1H, d, J=0.73 Hz); 8.05 (1H, d, J=0.36 Hz); 7.88 (2H, d, J=8.59 Hz); 7.61-7.54 (2H, m); 7.34-7.30 (1H, m); 4.36 (2H, t, J=6.95 Hz); 3.46-3.41 (2H, m); 2.59 (3H, s); 1.98-1.90 (2H, m). LCMS: Method D, 40-100%, Rₜ=6.75 min, [MH+=370.36].

### EXAMPLE 73

### Preparation of N-[3-(3-Methyl-4-oxo-1,4-dihydro-pyrazolo[4,3-c]quinolin-5-yl)-propyl]-benzamide

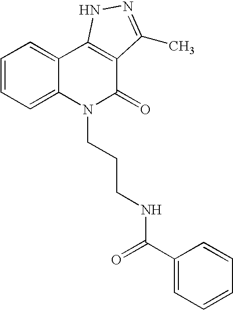

Using the appropriate reagent and in manner similar to that exemplified in Example 35 the title compound was obtained as a white solid. ¹H NMR (400 MHz, DMSO-d6) δ 8.48 (1H, t, J=5.6 Hz); 8.06 (1H, d, J=7.6 Hz); 7.79 (1H, m); 7.56-7.36 (5H, m); 7.24 (1H, t, J=7.0 Hz); 4.27 (2H, t, J=7.4 Hz); 3.33 (2H, q, J=6.5 Hz); 2.52 (3H, s); 1.84 (2H, m). LCMS: Method B, Rₜ=3.23 min, [MH+=361].

### EXAMPLE 74

### Preparation of Morpholine-4-carboxylic acid [3-(3-methyl-4-oxo-2,4-dihydro-pyrazolo[4,3-c]quinolin-5-yl)-propyl]-amide

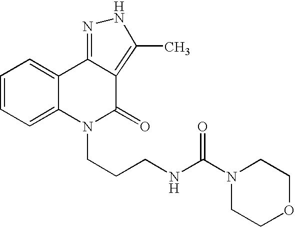

Using the appropriate reagents and in manner similar to that exemplified in Example 35 the title compound was obtained as a white solid. ¹H NMR (400 MHz, MeOD-d4) δ 8.02 (1H, d, J=8.1 Hz); 7.49-7.55 (2H, m); 7.22-7.27 (1H, m); 4.30 (2H, t, 7.0 Hz); 3.56 (4H, t, J=4.9 Hz); 3.28 (4H, t, 4.8 Hz); 2.58 (3H, s); 1.86 (2H, quintet, J=6.9 Hz).

LCMS: Method D, 40-60% B, Rₜ=6.75 min, [MH+=370.36].

### EXAMPLE 75

### Preparation of 4-Chloro-N-[3-(7-chloro-3-methyl-4-oxo-2,4-dihydro-pyrazolo[4,3-c]quinolin-5-yl)-propyl]-benzamide

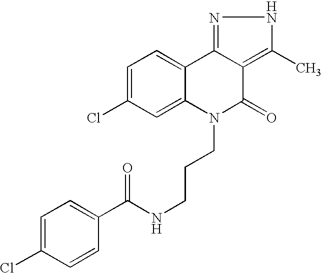

From 5-(3-Amino-propyl)-7-chloro-3-methyl-2,5-dihydro-pyrazolo[4,3-c]quinolin-4-one using the appropriate reagents and coupling conditions as in Example 35, the title compound was obtained as white solid. ¹H NMR (400 MHz, DMSO-d6) δ 13.8 (s, 1H, broad); 8.58 (dd, 1H, J=5.3, 4.2); 8.05 (d, 1H, J=8.4); 7.83-7.80 (d, 2H, J=8.4); 7.53 (d, 1H, broad, J=1.2); 7.48 (d, 2H, J=8.4); 7.29 (d, 1H, J=8.4); 4.25 (t, 2H, J=7.3); 3.34-3.28 (m, 2H); 2.51 (s, 3H); 1.85-1.78 (m, 2H). LCMS Method D, 40-100%, Rₜ=5.96 min [MH+=431/429].

### EXAMPLE 76

### Preparation of 3,4-Dichloro-N-[3-(3-methyl-4-oxo-2,4-dihydro-pyrazolo[4,3-c]quinolin-5-yl)-propyl]-benzamide

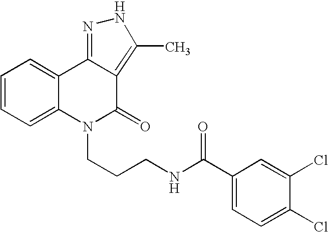

Using the appropriate reagents and in manner similar to that exemplified in Example 35 the title compound was obtained as a white solid. ¹H NMR (400 MHz, DMSO-d6) δ 8.72 (1H, t, J=5.3 Hz); 8.12 (1H, dd, J=7.7, 0.93 Hz); 8.08 (1H, d, J=2.0 Hz); 7.82 (1H, dd, J=8.4, 2.0 Hz); 7.76 (1H, d, J=8.4 Hz); 7.52-7.60 (2H, m); 7.30 (1H, t, J=7.0 Hz); 4.32 (2H, t, J=7.3 Hz); 3.39 (2H, quartet, J=6.4Hz); 2.57 (3H, s); 1.90 (2H, quintet, J=7.0 Hz). LCMS: Method C, 20-100% B, Rₜ=8.00 min, [MH+=429.23].

### EXAMPLE 77

### Preparation of N-[4-(8-Bromo-3-methyl-4-oxo-1,4-dihydro-pyrazolo[4,3-c]quinolin-5-yl)-butyl]-benzamide

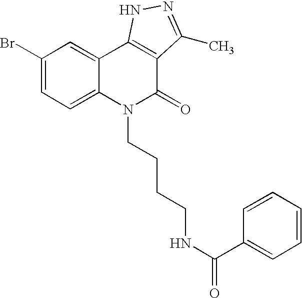

Using the appropriate reagents and in manner similar to that exemplified in Example 35 the title compound was obtained as a white solid. ¹H NMR (400 MHz, DMSO-d₆) δ 8.55 (1H, t, J=5.6 Hz); 7.88 (2H, d, J=7.1 Hz); 7.8-7.6 (4H, m); 7.58 (2H, t, J=7.2 Hz); 7.51 (2H, t, J=7.5 Hz); 4.32 (2H, m); 2.72-2.56 (2H, m); 2.57 (3H, s); 1.69 (4H, m). LCMS: Method B, Rₜ=3.66 min, [MH+=453/455].

### EXAMPLE 78

### Preparation of 4-Dimethylamino-N-[3-(3-methyl-4-oxo-2,4-dihydro-pyrazolo[4,3-c]quinolin-5-yl)-propyl]-benzamide

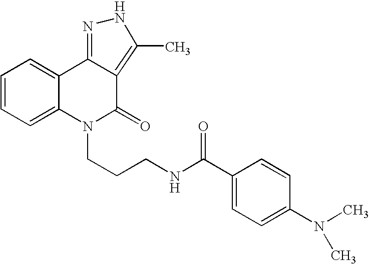

Using the appropriate reagents and in manner similar to that exemplified in Example 35 the title compound was obtained as a white solid. ¹H NMR (400 MHz, DMSO-d6) δ 8.25 (1H, t, broad); 8.1 (1H, dd, J=1.09, 7.5 Hz); 7.72 (2H, d, J=8.96 Hz); 7.53-7.47 (2H, m); 7.28-7.24 (1H, m); 6.77 (2H, d, J=8.23 Hz); 4.28 (2H, t, J=7.13 Hz); 3.31 (2H, dd, J=6.58, 11.70 Hz); 2.54 (3H, s); 1.88-180 (2H, m); 2.94 (6H, s). LCMS, Method B, Rₜ=3.63 min, MH+=404.32; MH−=402.41.

### EXAMPLE 79

### Preparation of [3-(8-Methoxy-3-methyl-4-oxo-2,4-dihydro-pyrazolo[4,3-c]quinolin-5-yl)-propyl]-carbamic acid tert-butyl ester

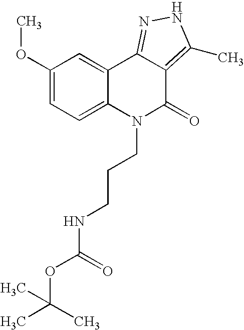

Prepared in Steps 1-2 using the procedure below, the other steps are as exemplified in Example 29.

### Step 1: Preparation of [3-(6-Methyl-2,4-dioxo-4H-benzo[d][1,3]oxazin-1-yl)-propyl]-carbamic acid tert-butyl ester

A suspension of 6-methyl-1H-benzo[d][1,3]oxazine-2,4-dione (859 mg, 4.8 mmol), triphenyl phosphine (1.65 g, 6.3 mmol) and (3-hydroxy-propyl)-carbamic acid tert-butyl ester (1.1 g, 6.3 mmol) in TBF (40 ml) is treated with diisopropyl azo dicarboxylate (DIAD) (1.24 ml, 6.3 mmol), and stirred for 2 hours at room temperature. After removal of solvent in vacuo the crude residue is dissolved in ethyl acetate (30 ml), hexane (50 ml) and ether (50 ml), and the precipitated triphenyl phosphine oxide filtered from the mixture of solvents. The combined filtrates are evaporated to dryness. The crude material was purified by flash chromatography (silica, 1:3 ethyl acetate/hexane) to provide [3-(6-Methyl-2,4-dioxo-4H-benzo[d][1,3]oxazin-1-yl)-propyl]-carbamic acid tert-butyl ester as an off white solid (1.0 g, 62% yield). ¹H NMR (400 MHz, DMSO-d6) δ 7.7 (1H, m); 7.6(1H, dd, 2.01, 8.59 Hz); 7.3 (1H, d, 8.59 Hz); 6.8 (1H, t, 7.03 Hz); 3.9 (2H, t, 7.68 Hz); 3.02-2.95 (2H, m); 2.3 (3H, s); 1.74-1.65 (2H, m); 1.32 (9H, s).

### Step 2: Preparation of 2-(3-tert-Butoxycarbonylamino-propylamino)-5-methyl-benzoic acid methyl ester

To a solution of [3-(6-methyl-2,4-dioxo-4H-benzo[d][1,3]oxazin-1-yl)-propyl]-carbamic acid tert-butyl ester (3 mmol, 1 eq, 1.0 g) in methanol (20 ml) was added sodium hydroxide (6 mmol, 2 eq, 0.240 g) dissolved in methanol (5 ml) and the reaction mixture refluxed for 3 hours. After cooling the solvent is removed in vacuo and the crude dissolved ethyl acetate. 1N HCl (4 ml) is added to pH 6-7, and the solution washed with in water and brine, dried over sodium sulphate, filtered and concentrated. The crude was purified by flash column chromatography in hexane/ethyl acetate 6:1 to afford 2-(3-tert-Butoxycarbonylamino-propylamino)-5-methyl-benzoic acid methyl ester as a white solid (0.665 g, 69% yield). ¹H NMR (400 MHz, DMSO-d6) δ 7.53 ppm(1H, d, J=2.01 Hz); 7.38 (1H, t, J=5.12 Hz); 7.15 (1H, dd, J=2.19, 8.59 Hz); 6.85 (1H, t, J=5.48 Hz); 6.61 (1H, d, J=8.78 Hz); 3.72 (3H, s); 3.12-3.08 (2H, dd, J=6.58, 12.44 Hz); 2.96-2.92 (2H, dd, J=6.58, 12.62 Hz); 2.10 (3H, s); 1.63-1.57 (2H, m); 1.31 (9H, s).

Steps 3 to the end afforded the title compound Example 79 as a white solid. ¹H NMR (400 MHz, DMSO-d6) δ 7.67 (1H, d, J=2.7 Hz); 7.45 (1H, d, J=9.33 Hz); 7.14 (1H,dd, 2.56, 9.3); 6.8 (2H, s, br); 4.17 (2H, m); 3.8 (3H, s); 2.97 (2H, dd, J=6.4, 6.03 Hz); 2.48 (3H, s); 1.68 (2H, m); 1.39( 9H,s). LCMS: Method A, Rₜ=2.85 min, [MH+=387].

### EXAMPLE 80

### Preparation of [3-(3-Methyl-4-oxo-2,4-dihydro-pyrazolo[4,3-c]quinolin-5-yl)-propyl]-carbamic acid 2-chloro-benzyl ester

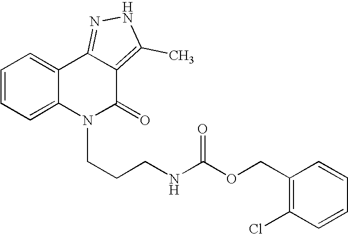

Using the appropriate reagents and in a manner similar to that exemplified in Example 35 the title compound was obtained as a white solid. ¹H NMR (400 MHz, DMSO-d6) δ 8.12(1H, d, J=7.6 Hz); 7.26 to 7.60(8H, mm, br); 5.10(2H, s); 4.26(2H, t, J=7.4 Hz); 3.15(2H, quartet, J=6.6 Hz); 2.58(3H, s, br); 1.78(2H, quintet, J=7.4 Hz).

LCMS: Method D, 40-100%, B, Rₜ=10.41 Min, [MH+=425.3].

### EXAMPLE 81

### Preparation of N-[3-(3-Methyl-4-oxo-2,4-dihydro-pyrazolo[4,3-c]quinolin-5-yl)-propyl]-4-phenyl-butyramide

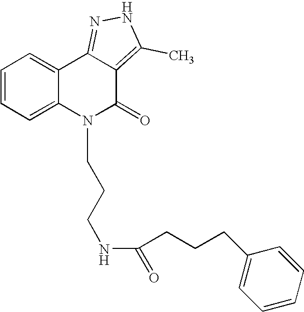

Using the appropriate reagent and in a manner similar to that exemplified in Example 35 the title compound was obtained as a white solid. ¹H NMR (400 MHz, DMSO-d6) δ 8.03 (1H, d, J=7.31 Hz); 7.81 (1H, t, J=5.48 Hz); 7.49 (1H, d, J=3.84 Hz); 7.41 (1H, s, broad); 7.27-7.16 (3H, m); 7.14-7.06 (3H, m); 4.21-4.14 (2H, m); 3.12-3.06 (2H, m); 2.55-2.44 (5H, m); 2.05-2.01 (2H, t, J=7.31 Hz); 1.17-1.64 (4H, m). LCMS: Method D, 40-100% B, Rₜ=9.99 min, MH+=403.43; MH−=401.46.

### EXAMPLE 82

### Preparation of N-[3-(3-Methyl-4-oxo-2,4-dihydro-pyrazolo[4,3-c]quinolin-5-yl)-propyl]-nicotinamide

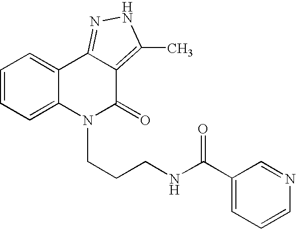

Using the appropriate reagent and in a manner similar to that exemplified in Example 35 the title compound was obtained as a white solid. ¹H NMR (400 MHz, DMSO-d6) δ 13.65 (1H, br s); 8.95 (1H, d, J=2.2 Hz); 8.68 (1H, t, J=5.5 Hz); 8.64 (1H, dd, J=4.8, 1.1 Hz); 8.12 (1H, dt, J=7.9, 1.1 Hz); 8.06 (1H, t, J=8.4 Hz); 7.52 (2H, m); 7.45 (1H, dd, J=8.0, 4.7 Hz); 7.25 (1H, br s); 4.29 (1H, t, J=6.6 Hz); 3.35 (2H, q, J=6.5 Hz); 2.50 (3H, br s); 1.86 (2H, quintet, J=7.1 Hz) LCMS: Method D, 40-100% B, Rₜ=6.98 min, [MH+=362.33].

### EXAMPLE 83

### Preparation of N-[3-(3-Methyl-4-oxo-2,4-dihydro-pyrazolo[4,3-c]quinolin-5-yl)-propyl]-benzenesulfonamide

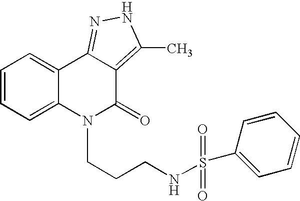

Using the appropriate reagent and in manner similar to that exemplified in Example 35 the title compound was obtained as a white solid. ¹H NMR (400 MHz, DMSO-d6) δ 8.09 (1H,dd, J=1.46, 7.86 Hz); 7.78-7.76 (2H, m); 7.63-7.48 (5H, m); 7.29 (1H, t, J=7.68 Hz); 4.19 (2H, t, J=7.50 Hz); 2.88-2.85 (2H, m); 2.55 (3H, s); 1.74-1.66 (2H, m). LCMS, Method D, 40-100% B, Rₜ=7.70 min, MH+=397.33; MH−=395.32.

### EXAMPLE 84

### Preparation of 2-Chloro-N-[3-(3-methyl-4-oxo-2,4-dihydro-pyrazolo[4,3-c]quinolin-5-yl)-propyl]-benzamide

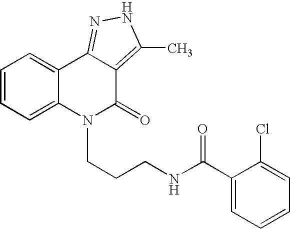

Using the appropriate reagent and in manner similar to that exemplified in Example 35 the title compound was obtained as a white solid. ¹H NMR (400 MHz, DMSO-d6) δ 8.46 (1H, t, J=6.40 Hz); 8.05 (1H, d, J=7.68 Hz); 7.55-7.49 (2H, m); 7.45-7.32 (4H, m); 7.25 (1H, t, J=7.31 Hz); 4.28 (2H, t, J=7.31 Hz); 3.33-3.28 (2H, m); 2.52 (3H, s) 1.86-1.79 (2H, m). LCMS, Method C, 20-100% B, Rₜ=6.3 min, MH+=395.33/398.4; MH−=393.24/396.33.

### EXAMPLE 85

### Preparation of 4-tert-Butyl-N-[3-(3-methyl-4-oxo-2,4-dihydro-pyrazolo[4,3-c]quinolin-5-yl)-propyl]-benzamide

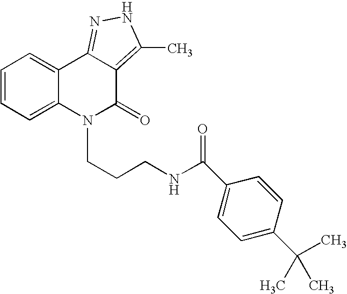

Using the appropriate reagent and in manner similar to that exemplified in Example 35 the title compound was obtained as a white solid. ¹H NMR (400 MHz, DMSO-d6) δ 13.70 (1H, br s); 8.47 (1H, t, J=5.6 Hz); 8.12 (1H, d, J=7.5 Hz); 7.76 (2H, d, J=8.5 Hz); 7.56 (2H, m); 7.48 (2H, d, J=8.4 Hz); 7.30 (1H, t, J=7.2 Hz); 4.33 (2H, t, J=7.2 Hz); 3.38 (2H, q, J=6.5 Hz); 2.58 (3H, s); 1.89 (2H, quintet, J=7.1 Hz); 1.30 (9H, s). LCMS: Method C, 40-100% B, Rₜ=6.40 min, [MH+=417].

### EXAMPLE 86

### Preparation of N-[3-(3-Methyl-4-oxo-2,4-dihydro-pyrazolo[4,3-c]quinolin-5-yl)-propyl]-acetamide

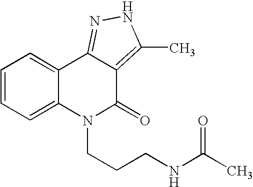

Using the appropriate reagents and in a manner similar to that exemplified in Example 35 the title compound was obtained as a white solid. ¹H NMR (400 MHz, DMSO-d6) δ 13.72 (1H, br s); 8.12 (1H, d, J=8.0 Hz); 7.94 (1H, t, J=5.7 Hz); 7.56 (2H, m); 7.31 (1H, t, J=6.8 Hz); 4.26 (2H, t, J=7.4 Hz); 3.15 (2H, q, J=6.5 Hz); 2.58 (3H, br s); 1.82 (3H, s); 1.74 (2H, quintet, J=7.3 Hz). LCMS: Method D, 40-100% B, Rₜ=5.94 min, [MH+=299].

### EXAMPLE 87

### Preparation of 1-[3-(3-Methyl-4-oxo-2,4-dihydro-pyrazolo[4,3-c]quinolin-5-yl)-propyl]-3-phenyl-urea

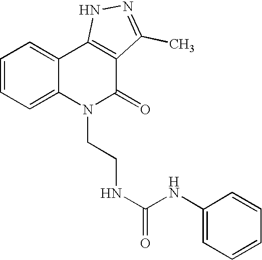

Starting from Example 11 and using the appropriate reagent and in a manner similar to that exemplified in Example 66 the title compound was obtained as a white solid. ¹H NMR (400 MHz, DMSO-d6) δ 8.58 (1H, s); 8.12 (1H, d, J=7.7 Hz); 7.88 (1H, m); 7.60 (1H, m); 7.40 (2H, d, J=8.0 Hz); 7.33 (1H, m); 7.23 (2H, t, J=7.7 Hz); 6.91 (1H, t, J=7.3 Hz); 6.41 (1H, t, J=5.6 Hz); 4.35 (2H, m); 3.36 (2H, q, J=6.7 Hz); [2.64 (s) and 2.55 (s), 3H]. LCMS: Method B, Rₜ=3.38 min, [MH+=362].

### NO EXAMPLE 88

### EXAMPLE 89

### Preparation of 1-(5-Chloro-2-methoxy-phenyl)-3-[2-(3-methyl-4-oxo-1,4-dihydro-pyrazolo[4,3-c]quinolin-5-yl)-ethyl]-urea

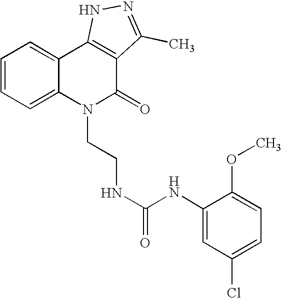

Starting from Example 11 and using the appropriate reagent and in a manner similar to that exemplified in Example 66 the title compound Was obtained as a white solid. ¹H NMR (400 MHz, DMSO-d6) δ 8.20 (1H, d, J=2.4 Hz); 8.13 (1H, s); 8.11 (1H, s); 7.79 (1H, br s); 7.61 (1H, br s); 7.31 (1H, br s); 7.23 (1H, t, J=5.8 Hz); 6.98 (1H, d, J=8.8 Hz); 6.92 (1H, dd, J=8.8, 2.6 Hz); 4.33 (2H, t, J=6.8 Hz); 3.36 (2H, q, J=6.7 Hz); 3.31 (3H, s)1 2.58 (3H, br s). LCMS: Method B, Rₜ=3.66 min, [M=424/426].

### EXAMPLE 90

### Preparation of 4-Chloro-N-[2-(3-methyl-4-oxo-2,4-dihydro-pyrazolo[4,3-c]quinolin-5-yl)-ethyl]-benzamide

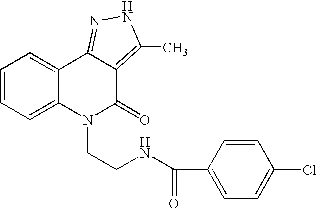

Starting from Example 11 and appropriate reagents and in a manner similar to that exemplified in Example 35 the title compound was obtained as a white solid.

¹H NMR (400 MHz, DMSO-d6) δ 8.81 (1H, t, J=5.7 Hz); 8.04 (1H, dd, J=7.7, 0.9 Hz); 7.78 (1H, d, J=8.8 Hz); 7.75 (2H, d, J=8.5 Hz); 7.50 (1H, t, J=8.1 Hz); 7.47 (2H, d, J=7.4 Hz); 7.23 (1H, t, J=7.5 Hz); 4.34 (2H, t, J=6.9 Hz); 3.47 (2H, m); 2.50 (3H, s). LCMS: Method D, 40-100% B, Rₜ=9.46 min, [MH+=381.31].

### EXAMPLE 91

### Preparation of 4-Methoxy-N-[2-(3-methyl-4-oxo-2,4-dihydro-pyrazolo[4,3-c]quinolin-5-yl)-ethyl]-benzamide

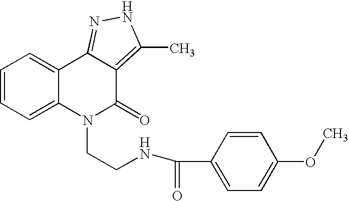

Starting from Example 11 and appropriate reagents, and in a manner similar to that exemplified in Example 35 the title compound was obtained as a white solid. ¹H NMR (400 MHz, DMSO-d6) δ 8.49 (1H, t, J=5.8 Hz); 7.93 (1H, dd, J=7.8, 1.3 Hz); 7.73 (1H, d, J=8.7 Hz); 7.62 (2H, d, J=8.8 Hz); 7.39 (1H, t, J=7.6 Hz); 7.12 (1H, t, J=7.5 Hz); 6.81 (2H, d, J=8.8 Hz); 4.20 (2H, t, J=7.3 Hz); 3.62 (3H, s); 3.31 (2H, m); 2.39 (3H, s). LCMS: Method D, 40-100% B, Rₜ=8.34 min, [MH+=377.32].

### EXAMPLE 92

### Preparation of N-[2-(3-Methyl-4-oxo-2,4-dihydro-pyrazolo[4,3-c]quinolin-5-yl)-ethyl]-3-phenyl-propionamide

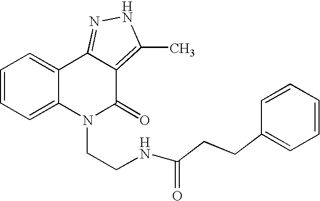

Starting from Example 11 and appropriate reagents, and in a manner similar to that exemplified in Example 35, the title compound was obtained as a white solid.

¹H NMR (400 MHz, DMSO-d6) δ 8.24-8.19 (1H, m); 8.15 (1H, dd, J=1.28, 7.68 Hz); 7.83(1H, d, J=8.59 Hz); 7.65-7.59 (1H, m); 7.37-7.18 (5H, m); 4.26 (2H, t, J=7.31 Hz); 3.34-3.30 (2H, m); 2.82 (2H, t, J=7.68 Hz); 2.55-2.53 (2H, m). LCMS, Method D, 40-100% B, Rₜ=8.82 min, MH+=375.38; MH−=373.41.

### EXAMPLE 93

### Preparation of Cyclohexanecarboxylic acid [2-(3-methyl4-oxo-2,4-dihydro-pyrazolo[4,3-c]quinolin-5-yl)-ethyl]-amide

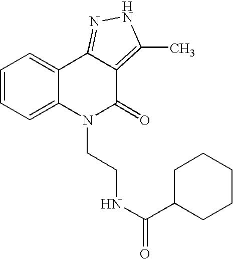

Starting from Example 11 and appropriate reagents, and in a manner similar to that exemplified in Example 35 the title compound was obtained as a white solid. ¹H NMR (400 MHz, CDCl₃) δ 8.30 (1H, d, J=7.3 Hz); 7.78 (1H, d, J=8.8 Hz); 7.44 (2H, m); 7.30 (1H, t, J=7.6 Hz); 4.4 (2H, t, J=6.1 Hz); 4.2 (1H, m) 3.5 (2H, t, J=6.5 Hz); 2.82 (3H, s); 1.80-1.96 (2H, m); 1.63-1.80 (4H, m); 1.50-1.58(4H, m). LCMS: Method D, 40-60% B, Rₜ=9.04 min, [MH+=353].

### EXAMPLE 94

### Preparation of Cyclopentanecarboxylic acid [2-(3-methyl-4-oxo-2,4-dihydro-pyrazolo[4,3-c]quinolin-5-yl)-ethyl]-amide

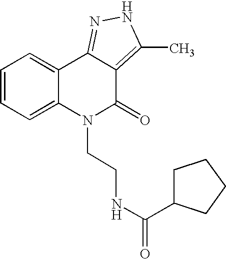

Starting from Example 11 and appropriate reagents, and in a manner similar to that exemplified in Example 35 the title compound was obtained as a white solid. ¹H NMR (400 MHz, MeOH-d4) δ 8.08 (1H, s, br); 7.80 (1H, d, br, J=7.1 Hz); 7.60 (1H, s, br); 7.3 (1H, t, br, J=7.5 Hz); 4.4 (2H, t, J=6.6 Hz); 3.5 (2H, t, J=6.7 Hz); 2.6 (3H, s); 2.5 (1H, quintet, J=7.6 Hz); 1.70-1.77 (2H, m); 1.48-1.68 (6H, m). LCMS: Method D, 40-100% B, Rₜ=8.33 min, [MH+=339].

### EXAMPLE 95

### Preparation of {2-(4-Fluoro-phenyl)-1-[3-(3-methyl-4-oxo-2,4-dihydro-pyrazolo[4,3-c]quinolin-5-yl)-propylcarbamoyl]-ethyl}-carbamic acid tert-butyl ester

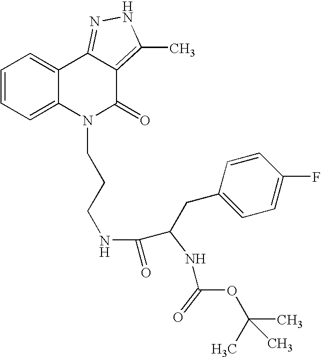

Dry DMF (2 ml) and DIPEA (100 ul) were added to the amine hydrochloride (Example 15) (52.5 mg, 0.179 mmol) and the mixture was sonicated and then stirred for 15 min to produce a white suspension. A solid mixture of the amino acid derivative, Boc-Phe(4-F)—OH (101.6 mg, 0.359 mmol) and HOBt (68.6 mg, 0.448 mmol) was added to the amine suspension and after brief mixing, the coupling was induced by the addition of solid EDC (68.7 mg, 0.3587 mmol). The suspension was vigorously mixed at room temperature, whereupon the suspension completely cleared within 15 min. After mixing for 2 h or overnight, tris-(2-aminoethyl)-amine polystyrene (200-400 mesh, ca. 0.34 mmole/g, ca. 100 mg, swelled in DCM and washed with DCM then DMF) was added to the reaction mixture and stirring continued for 2 h or overnight. The scavenger resin was removed by filtration and rinsed with DMF then ethyl acetate and the combined organic filtrates were distributed between ethyl acetate (180 ml) and water (50 ml). The separated organic layer was washed with 30-50 ml portions of water, 4-times; 0.2M HCl, 3-times; water, 1-time; sat. NaHCO₃, 4-times and then with water, 4-times. After evaporation of the ethyl acetate and re-evaporation from methanol, 2-times, the target amide was triturated 3-times with ether and dried in vacuo to give the title compound as a white solid (84.4 mg; yield 90.2%). ¹H NMR (400 MHz, DMSO-d6) δ 13.70 (1H, br s); 8.12 (1H, d, J=7.3 Hz); 8.03 (1H, t, J=5.4 Hz); 7.55 (2H, m); 7.29 (3H, m); 7.07 (2H, t, J=8.9 Hz); 6.96 (1H, d, J=8.4 Hz); 4.24 (2H, m); 4.11 (1H, m); 3.18 (2H, m); 2.96 (1H, dd, J=13.7, 4.6 Hz); 2.75 (1H, dd, J=13.7, 10.1 Hz); 2.58 (3H, br s); 1.76 (2H, m); 1.29 (9H, s). LCMS, Method C, 40-100% B, Rₜ=5.66 min, MH+=522.29; MH+-Boc=422.28.

### EXAMPLE 96

### Preparation of {2-(4-Chloro-phenyl)-1-[3-(3-methyl-4-oxo-2,4-dihydro-pyrazolo[4,3-c]quinolin-5-yl)-propylcarbamoyl]-ethyl}-carbamic acid tert-butyl ester

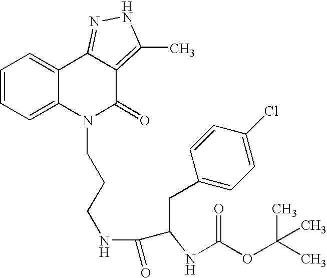

Using the appropriate reagents and in a manner similar to that exemplified in Example 95 the title compound was obtained as a white solid. ¹H NMR (400 MHz, DMSO-d6) δ 8.33 (1H, d, J=7.7 Hz); 8.26 (1H, t, J=5.6 Hz); 7.85-7.70 (2H, m); 7.6-7.4 (5H, m); 7.19 (1H, d, J=8.4 Hz); 4.44 (2H, m); 4.33 (1H, m); 3.39 (2H, m); 3.16 (1H, m); 2.97 (1H, m); 2.76 (3H, br s); 1.95 (2H, m); 1.51 (9H, s). LCMS, Method D, 40-100%, Rₜ=10.56 min, MH+=538.47; MNa+=560.43; MH+-Boc=438.39.

### EXAMPLE 97

### Preparation of {1-[2-(3-Methyl-4-oxo-1,4-dihydro-pyrazolo[4,3-c]quinolin-5-yl)-ethylcarbamoyl]-2-phenyl-ethyl}-carbamic acid tert-butyl ester

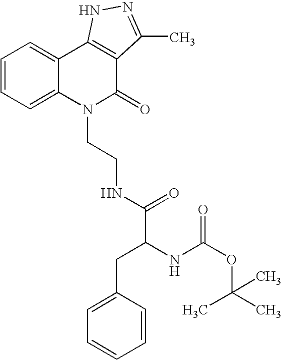

Using the appropriate reagent and in manner similar to that exemplified in Example 95, the title compound was obtained as a white solid. ¹H NMR (400 MHz, DMSO-d6) δ 13.7 (1H, br s); 8.23 (1H, t, J=5.3 Hz); 8.11 (1H, d, J=7.4 Hz); 7.80 (1H, d, J=8.6 Hz); 7.59 (1H, t, J=7.4 Hz); 7.31 (1H, t, J=7.4 Hz); 7.23 (4H, m); 7.17 (1H, t, J=6.7 Hz); 6.87 (1H, d, J=8.5 Hz); 4.24 (2H, m); 4.10 (1H, m); 3.41 (1H, m); 3.25 (1H, m); 2.91 (1H, dd, J=13.7, 4.6 Hz); 2.69 (1H, dd, J=13.5, 10.1 Hz); 2.56 (3H, s); 1.30 (9H, s). LCMS: Method B, Rₜ=3.59 min, [MH+=490].

### EXAMPLE 98

### Preparation of {2-(4-tert-Butoxy-phenyl)-1-[3-(3-methyl-4-oxo-2,4-dihydro-pyrazolo[4,3-c]quinolin-5-yl)-propylcarbamoyl]-ethyl}-carbamic acid tert-butyl ester

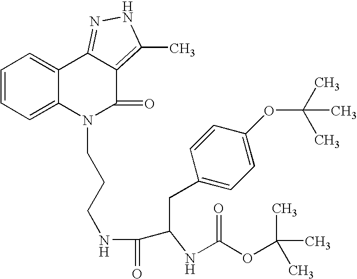

Using the appropriate reagents and in a manner similar to that exemplified in Example 95, the title compound was obtained as a white solid. ¹H NMR (400 MHz, DMSO-d6) δ 8.03 (1H, d, J=7.7 Hz); 7.95 (1H, t, J=5.8 Hz); 7.50 (1H, m); 7.43 (1H, m); 7.25 (1H, m); 7.06 (2H, d, J=8.4 Hz); 6.89 (2H, d, J=8.4 Hz); 6.75 (2H, d, J=8.3 Hz); 4.14 (2H, m); 4.03 (1H, m); 3.08 (2H, m); 2.84 (1H, dd, J=13.5, 4.7 Hz); 2.63 (1H, dd, J=13.6, 10.2 Hz); [2.56 (s) and 2.46 (s) 3H]; 1.64 (2H, m); 1.21 (9H, s); 1.12 (9H, s).

### EXAMPLE 99

### Preparation of 2-tert-Butoxycarbonylamino-4-[3-(3-methyl-4-oxo-2,4-dihydro-pyrazolo[4,3-c]quinolin-5-yl)-propylcarbamoyl]-butyric acid tert-butyl ester

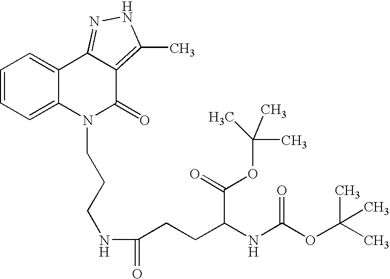

Using the appropriate reagents and in a manner similar to that exemplified in Example 95, the title compound was obtained as a white solid. ¹H NMR (400 MHz, DMSO-d6) δ 8.11 (1H, d, J=7.68 Hz); 7.92 (1H, m); 7.59-7.52 (2H, m); 7.3 (1H, m); 7.13 (1H, d, J=7.68 Hz); 4.24 (2H, t, J=7.68 Hz); 3.81-3.73 (1H, m); 3.18-3.11 (2H, m); 2.57 (3H, s); 2.16 (2H, t, J=7.13 Hz); 1.94-1.69 (4H,m); 1.38 (9H,s); 1.36 (9H,s). LCMS, Method D, 40-100%, Rₜ=10.3 min, MH+=542.52; MH−=540.61

### EXAMPLE 100

### Preparation of {3-[3-(3-Methyl-4-oxo-2,4-dihydro-pyrazolo[4,3-c]quinolin-5-yl)-propylcarbamoyl]-propyl}-carbamic acid tert-butyl ester

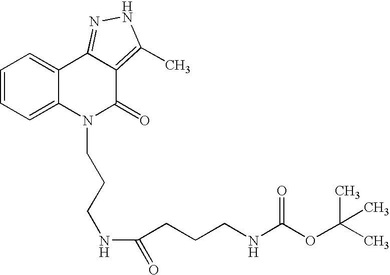

Using the appropriate reagents and in a manner similar to that exemplified in Example 95, the title compound was obtained as a white solid. ¹H NMR (400 MHz, DMSO-d6) δ 7.87 (1H, d, J=8.05 Hz); 7.64 (1H, t, J=5.3 Hz); 7.38-7.23 (2H, m); 7.11-6.99 (1H, m); 6.54 (1H, m); 4.05-3.96 (2H, m); 2.95-2.89 (2H, m); 2.70-2.64 (2H, m); 2.27-2.25 (3H, m); 1.83 (2H, t, J=7.31 Hz); 1.54-1.47 (2H,m); 1.40-1.33 (2H,m); 1.12 (9H,s). LCMS, Method D, 40-100%, Rₜ=8.81 min, MH+=442.49; MH+-Boc=342.4; MH−=440.49; MH− -tBuOH=366.37.

### EXAMPLE 101

### Preparation of {2-[3-(3-Methyl-4-oxo-2,4-dihydro-pyrazolo[4,3-c]quinolin-5-yl)-propylcarbamoyl]-ethyl}-carbamic acid tert-butyl ester

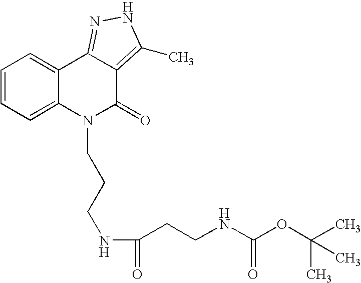

Using the appropriate reagent and in a manner similar to that exemplified in Example 95, the title compound was obtained as a white solid. ¹H NMR (400 MHz, DMSO-d6) δ 8.12(1H, dd, J=7.9 Hz); 7.95(1H, t, J=5.8 Hz); 7.52 to 7.62 (2H, m); 7.31(1H, t, J=7.1 Hz); 6.76(2H, t, J=7.7 Hz); 4.22(2H, t, J=7.7 Hz); 3.14(4H, m); 2.59(3H, s); 2.24(2H, t, J=7.1 Hz); 1.74(2H, quintet, J=7.3 Hz); 1.34(9H, s). LCMS, Method D, 40-100% B, Rₜ=8.58 min MNa+=450.39.

### EXAMPLE 102

### Preparation of {[3-(3-Methyl-4-oxo-2,4-dihydro-pyrazolo[4,3-c]quinolin-5-yl)-propylcarbamoyl]-methyl}-carbamic acid tert-butyl ester

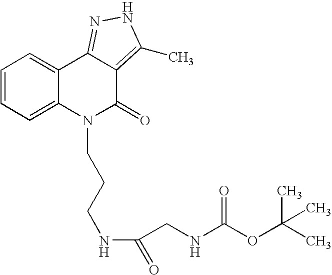

Using the appropriate reagents and in a manner similar to that exemplified in Example 95, the title compound was obtained as a white solid. ¹H NMR (400 MHz, DMSO-d6) δ 13.70 (1H, br s); 8.12 (1H, d, J=8.2 Hz); 7.90 (1H, t, J=5.6 Hz); 7.56 (2H, m); 7.31 (1H, t, J=6.7 Hz); 6.98 (1H, t, J=5.7 Hz); 4.26 (2H, t, J=7.2 Hz); 3.53 (2H, d, J=6.0 Hz); 3.17 (2H, q, J=6.3 Hz); 2.58 (3H, s); 1.76 (2H, m); 1.39 (9H, s). LCMS: Method D, 40-100% B Rₜ=8.45 min, [MH+=414.42].

### EXAMPLE 103

### Preparation of 5-Oxo-pyrrolidine-2-carboxylic acid [3-(3-methyl-4-oxo-2,4-dihydro-pyrazolo[4,3-c]quinolin-5-yl)-propyl]-amide

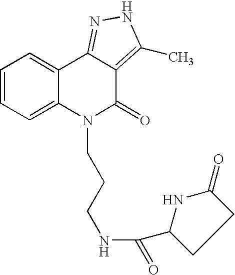

Using the appropriate reagents and in a manner similar to that exemplified in Example 95, the title compound was obtained as a white solid. ¹H NMR (400 MHz, DMSO-d6) δ 7.87 (1H, d, J=7.86 Hz); 7.81 (1H, t, J=5.85 Hz); 7.54 (1H, s); 7.35-7.29 (2H, m); 7.06 (1H, t, J=7.86 Hz); 4.02 (2H, t, J=7.68 Hz); 3.75-3.71 (1H, dd, J=4.57, 8.59 Hz); 2.98-2.91 (2H, s); 2.33 (3H, m); 2.05-1.80 (3H, m); 1.69-1.61 (1H, m); 1.57-1.50 (2H, m). LCMS, Method C, 5-60% B, Rₜ=6.84 min, MH+=368.25; MH−=366.24.

### EXAMPLE 104

### Preparation of 2-Amino-3-(4-fluoro-phenyl)-N-[3-(3-methyl-4-oxo-2,4-dihydro-pyrazolo[4,3-c]quinolin-5-yl)-propyl]-propionamide

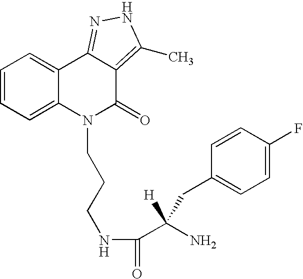

The N-Boc-derivative (Example 95) (73 mg, 0.140 mmole) was treated with 50% TFA in DCM (10 ml) for 90 min. The reaction solution was evaporated and the product was isolated after re-evaporation from methanol, 2-times; re-evaporation from 1.25M HCl in methanol (1 ml) in methanol (ca. 10 ml), 2-times; re-evaporation from methanol, 2-times, and finally by washing 3-times with ether and drying to give the title compound as a white solid (58 mg; yield 90.5%). ¹H NMR (400 MHz, DMSO-d6) δ 8.62 (1H, m); 8.32 (2H, m); 8.17 (1H, d, J=7.9 Hz); 7.58 (2H, m); 7.32 (3H, m); 7.15 (2H, t, J=8.8 Hz); 4.25 (2H, m); 4.00 (1H, m); 3.26 (1H, m); 3.20 (1H, m); 3.11 (1H, dd, J=13.7, 6.7 Hz); 3.03 (1H, dd, J=13.9, 7.3 Hz); 2.60 (3H, s); 1.75 (2H, m). LCMS: Method D, 20-100% B, Rₜ=9.47 min, MH+=422.28.

### EXAMPLE 105

### Preparation of 2-Amino-N-[2-(3-methyl-4-oxo-1,4-dihydro-pyrazolo[4,3-c]quinolin-5-yl)-ethyl]-3-phenyl-propionamide

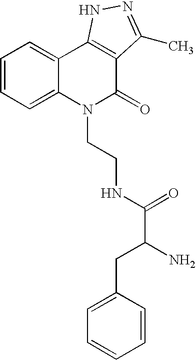

Using the appropriate reagents and in a manner similar to that exemplified in Example 104, the title compound was obtained as a white solid. ¹H NMR (400 MHz, DMSO-d6) δ 8.81 (1H, t, J=5.7 Hz); 8.35-8.2 (2H, m); 8.15 (1H, dd, J=7.8, 1.2 Hz); 7.76 (1H, d, J=8.8 Hz); 7.60 (1H, t, J=8.4 Hz); 7.35-7.2 (6H, m); 4.21 (2H, m); 3.93 (1H, m); 3.50 (1H, m); 3.22 (1H, m); 3.05 (1H, dd, J=13.8, 7.3 Hz); 2.90 (1H, dd, J=14.0, 7.9 Hz); 2.53 (3H, s). LCMS: Method B, Rₜ=3.13 min, [MH+=390].

### EXAMPLE 106

### Preparation of 4-Amino-N-[3-(3-methyl-4-oxo-2,4-dihydro-pyrazolo[4,3-c]quinolin-5-yl)-propyl]-butyramide

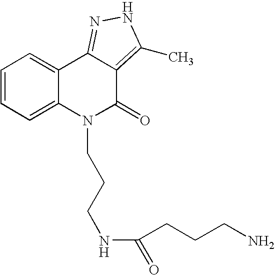

Using the appropriate reagents and in a manner similar to that exemplified in Example 104, the title compound was obtained as a white solid. ¹H NMR (400 MHz, DMSO-d6) ™: 8.15 (1H, dd, J=7.5, 1.3 Hz); 8.10 (1H, t, J=5.9 Hz); 7.9 (2H, s, br); 7.52-7.60 (2H, m); 7.29-7.34 (1H, m); 4.26 (2H, t, J=7.3 Hz); 3.19(2H, quartet, J=6.5 Hz); 2.80 (2H, m); 2.58 (3H, s); 2.22 (2H, t, J=7.1 Hz); 1.71-1.82(4H, m). LCMS, Method D, 20-100% B, Rₜ=6.78 min, MH+=342.42; MH+-NH₃=325.

### EXAMPLE 107

### Preparation of 2-Amino-3-(4-benzyloxy-phenyl)-N-[3-(3-methyl-4-oxo-2,4-dihydro-pyrazolo[4,3-c]quinolin-5-yl)-propyl]-propionamide

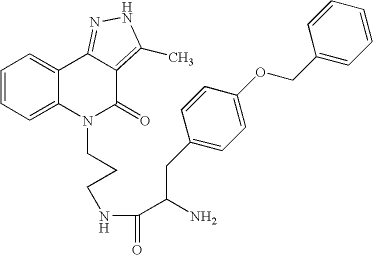

Using the appropriate reagents and in a manner similar to that exemplified in Example 104, the title compound was obtained as a white solid. ¹H NMR (400 MHz, DMSO-d6) δ 8.49 (1H, t, J=5.5 Hz); 8.23 (2H, dd, J=16.0, 5.1 Hz); 8.13 (1H, dd, J=7.7, 1.5 Hz); 7.50-7.60 (2H, m); 7.26-7.34 (6H, m); 7.16 (2H, d, J=8.6 Hz); 6.92 (2H, d, J=8.6 Hz); 4.87 (2H,s); 4.08-4.24 (2H, m); 3.92 (1H, m, br); 3.26 (1H, m); 3.14 (1H, m); 2.97 (2H, m); 2.56 (3H, s); 1.70 (2H, m). LCMS, Method D, 40-100% B, Rₜ=9.39 min, MH+=510.4; MH−=508.4.

### EXAMPLE 108

### Preparation of 2-Amino-3-(4-hydroxy-phenyl)-N-[3-(3-methyl-4-oxo-2,4-dihydro-pyrazolo[4,3-c]quinolin-5-yl)-propyl]-propionamide

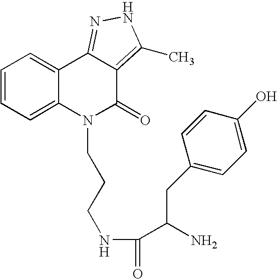

Using the appropriate reagents and in a manner similar to that exemplified in Example 104, the title compound was obtained as a white solid. ¹H NMR (400 MHz, DMSO-d6) δ 8.34 (1H, t, J=5.5 Hz); 8.05-7.85 (3H, m); 7.39 (2H, m); 7.14 (1H, t, J=7.7 Hz); 6.87 (2H, d, J=8.4 Hz); 6.52 (2H, d, J=8.4 Hz); 4.06 (2H, m); 3.71 (1H, m); 3.03 (2H, m); 2.80 (1H, dd, J=14.1, 6.6 Hz); 2.70 (1H, dd, J=14.1, 7.3 Hz); 2.40 (3H, s); 1.58 (2H, quintet, J=7.0 Hz). LCMS, Method D, 20-100% B, Rₜ=7.83 min, MH+=420.09; MH−=418.27.

### EXAMPLE 109

### Preparation of 5-(3-Amino-propyl)-8-chloro-2,5-dihydro-pyrazolo[4,3-c]quinolin-4-one (route 3)

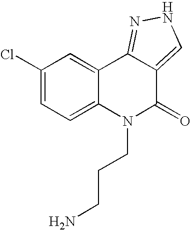

### Step 1: Preparation of 1-(Tetrahydro-pyran-2-yl)-1H-pyrazole-4-carboxylic acid

To a solution of 4-pyrazole carboxylic acid (6.3 mmol, 947 mg) in EtOAc/DMF (50/5 ml) at room temperature was added 3,4-dihydro-2H-pyran (12.45 mmoles, 1.135 ml) followed by para-toluenesulfonic acid (0.1 eq, 79 mg). The mixture was stirred for 3 hours. Upon completion, the reaction mixture was concentrated under vacuum and the residue partitioned between saturated aqueous sodium carbonate (150 ml) and EtOAc (50 ml), the aqueous layer was decanted and acidified to pH 5 then extracted with EtOAc (4×100 ml). The EtOAc layers were combined, dried over Na₂SO₄ and concentrated to afford the title compound as a white solid (1.46 g, 90%). ¹H NMR (400 MHz, DMSO-d6) δ 12.44 (s, 1H, Broad); 8.36 (s, 1H, Broad); 7.84 (s, 1H); 5.44 (dd, 1H, J=9.9, 2.1); 3.96-3.91 (m, 1H); 3.65-3.59 (m, 1H); 2.15-2.05 (m, 1H); 1.94-1.87 (m, 2H); 1.71-1.60 (m, 1H); 1.56-1.48 (m, 1H). LCMS: method A, Rₜ=1.95 min, [MH⁺=197].

### Step 2: Preparation of 1-(Tetrahydro-pyran-2-yl)-1H-pyrazole-4-carbonyl fluoride

To a suspension of 1-(tetrahydro-pyran-2-yl)-1H-pyrazole-4-carboxylic acid (3.6 mmoles, 1 eq, 700 mg) in dichloromethane (23 ml) at 0° C. was added pyridine (1.3 moles, 3 eq, 0.90 ml) followed by cyanuric fluoride (10.82 mmoles, 3 eq, 0.915 mL) and the slurry stirred at room temperature for 2 hours. Upon completion the reaction medium was quenched with iced water (20 ml) and diluted with DCM (100 ml). The aqueous layer was extracted with DCM (2×60 ml). The combined chlorinated layers were successively washed with H₂O (2×20 ml) dried over MgSO₄ and concentrated under vacuum to afford the acid fluoride as crude oily solid (669 mg). ¹H NMR (400 MHz, CDCl₃) δ 8.85 (s, 0.8H); 8.18 (s, 0.2H); 8.22 (s, 0.8H); 8.20 (s, 0.2H); 5.58 (ddₐₚₚₐᵣₑₙₜ, 1H, J=9.1, 2.5); 4.04-3.98 (m, 1H); 3.74-3.68 (m, 1H); 2.21-2.11 (m, 1H); 2.03-1.96 (m,2H); 1.90-1.67 (m, 1H); 1.63-1.57 (m, 2H).

### Step 3: Preparation of 1-(Tetrahydro-pyran-2-yl)-1H-pyrazole-4-carboxylic acid (2-bromo-4-chloro-phenyl)-amide

To as suspension of sodium hydride (60% in mineral oil, 1.2 mmol, 183 mg) in DMF (10 ml) was added dropwise, at room temperature a solution of 4-bromo-4-chloroaniline (4.95 mmol, 1.019 g). The mixture was stirred until no hydrogen evolution was observed (1 h), after which a solution of the acid fluoride (3.3 mmol, 646 mg) in DMF (10 ml) was added. The reaction mixture was heated at 90° C. for 12 hours. Upon completion the black mixture was concentrated and the residue partitioned between H₂O (80 ml) and EtOAc (120 ml). the aqueous layer was decanted and extracted with EtOAc (3×100 ml). The combined EtOAc layers were successively washed with brine, dried over Na₂SO₄, filtered and concentrated under vacuum to afford the crude product (1.2 g). Purification by flash chromatography (75/25: Isohexane/EtOAc) afforded the title compound as a viscous oil (380 mg, 30%). ¹H NMR (400 MHz, CDCl₃) δ 8.36(d, 1H, J=8.8); 8.10(s, 1H); 7.90(s, 1H); 7.49(d, 1H, J=2.4); 7.25(dd, 1H, J=8.9, 2.7); 5.37(dd, 1H, 9.0, 3.1); 4.043-3.99(m, 1H); 3.69-3.63(m, 1H); 2.10-1.91(m, 2H); 1.71-1.56(m, 2H).

### Step 4: Preparation of (3-{(2-Bromo-4-chloro-phenyl)-[1-(tetrahydro-pyran-2-yl)-1H-pyrazole-4-carbonyl]-amino}-propyl)-carbamic acid tert-butyl ester

To a solution of 1-(Tetrahydro-pyran-2-yl)-1H-pyrazole-4-carboxylic acid (2-bromo-4-chloro-phenyl)-amide (0.97 mmol, 374 mg) in DMF (20 ml) at RT was added in one portion tBuOK (0.97 mmol, 110 mg), the resulting dark brown solution was stirred for 3 minutes before the successive addition of K₂CO₃(3.9 mmol, 538 mg) and the alkyl halide (2.43 mmol, 576 mg). The reaction mixture was heated at 90° C. for 12 hours. The crude mixture was concentrated and the residue partitioned between H₂O (100 ml) and EtOAc (100 ml). The aqueous layer was decanted and extracted with EtOAc (3×100 ml). The combined EtOAc layers were successively washed with saturated ammonium chloride aqueous solution, dried over Na₂SO₄ and concentrated under vacuum to afford the crude product (777 mg). Purification by flash chromatography (75/25 to 50/50: Isohexane/EtOAc) afforded the title compound as a viscous oil (361 mg, yield=70%). ¹H NMR (400 MHz, CDCl₃) δ 7.64(dd, 1H, J=4.2, 2.4); 7.55(s, 1H, broad); 7.32-7.28(m, 1H); 7.19(s, 1H); 7.13(d. 1H, J=9.4); 6.60(s, 1H, broad); 5.19-5.15(m, 1H); 4.15-4.24(m, 1H); 3.91-3.85(m, 1H); 3.60-3.53(m, 1H); 3.40-3.20(m, 2H); 3.06-2.97(m, 1H); 1.99-1.84(m, 2H); 1.70-1.50(m, 6H); 1.36(s, 9H).

### Step 5: Preparation of {3-[8-Chloro-4-oxo-1-(tetrahydro-pyran-2-yl)-1,4-dihydro-pyrazolo[4,3-c]quinolin-5-yl]-propyl}-carbamic acid tert-butyl ester

To a degassed and nitrogen flushed suspension of (3-{(2-bromo-4-chloro-phenyl)-[1-(tetrahydro-pyran-2-yl)-1H-pyrazole-4-carbonyl]-amino}-propyl)-carbamic acid tert-butyl ester (0.414 mmol, 225 mg), tetraethyl ammonium chloride hydrate (0.41 mmol, 69 mg), and KOAc (2.10 mmol, 207 mg) in DMF (10 ml), was added Pd(OAc)₂ (0.22 eq, 26 mg). The mixture was heated at 90° C. for 2 hours. The crude mixture was concentrated and the residue partitioned between H₂O (30 ml) and DCM (40 ml). The aqueous layer was decanted and extracted with DCM (5×40 ml). The combined chlorinated layers were successively washed with brine, dried over Na₂SO₄, filtered and concentrated under vacuum to afford the crude product (200 mg). Purification by flash chromatography (75/25 to 50/50: Isohexane/AcOEt) afforded the title compound as a white solid (156 mg, 68%). ¹H NMR (400 MHz, CDCl₃) δ 8.17(s, 1H, broad); 8.08(d, 1H, J=2.2); 7.47(dd, 1H, J=9.1, 2.3); 7.34(d, 1H, 9.1); 7.26(m, 1H); 5.77(dd, 1H, J=8.4, 3.1); 4.36(t, 1H, J=6.6); 3.97(dt, 1H, J=11.3, 3.4); 3.82-3.76(m, 1H); 3.05(td, 2H, J1=J2=6.2); 2.618-2.54(m, 1H); 2.254-2.213(m, 2H); 1.86(m, 2H); 1.76-1.68(m, 3H); 1.38(s, 9H). LCMS: method A, Rₜ=3.75 min, [MH⁺=461].

Step 6: {3-[8-Chloro-4-oxo-1-(tetrahydro-pyran-2-yl)-1,4-dihydro-pyrazolo[4,3-c]quinolin-5-yl]-propyl}-carbamic acid tert-butyl ester (50 mg, 0.108 mmol) was treated with a trifluoroacetic (TFA)/ H₂O, 50/50 (2.5 ml) for 4 hours. Upon completion the reaction mixture was concentrated under vacuum and the obtained oily residue was co-evaporated several times with a solution of HCl 1.25M in MeOH to afford the title compound (5-(3-Amino-propyl)-8-chloro-2,5-dihydro-pyrazolo[4,3-c]quinolin-4-one) as a white solid (36 mg). ¹H NMR (400 MHz, DMSO-d6) δ 8.26 (s, broad, 1H); 8.06 (s, broad, 1H); 7.8-7.6 (s, broad, 2H); 7.54 (d, 1H, J=9.1); 7.46 (dd, 1H, J=9.1, 2.3); 4.18 (t, 2H, J=7.1); 2.74-2.67 (m, 2H); 1.80-1.73 (m, 2H). LCMS: Method A, 5-60%B, Rₜ=5.61 min [MH+=277/275].

### EXAMPLE 110

### Preparation of N-[2-(3-Methyl-4-oxo-2,4-dihydro-pyrazolo[4,3-c]quinolin-5-yl)-ethyl]-benzene sulfonamide

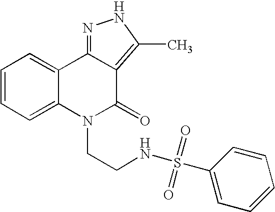

Starting from Example 11 and the appropriate reagents, and in a manner similar to that exemplified in Example 35 the title compound was obtained as a white solid. ¹H NMR (400 MHz, DMSO-d6) δ 8.09 (1H, dd, J=7.7, 1.3 Hz); 7.76 (2H, d, J=7.5 Hz); 7.44-7.62 (6H, m); 7.3 (1H, t, J=7.2 Hz); 4.28 (2H, t, J=7.3 Hz); 3.02 (2H, t, J=7.1 Hz); 2.55 (3H, s, br). LCMS, Method D, 40-100% B, Rₜ=6.78 min, MH+=383.27.

### NO EXAMPLE 111

### EXAMPLE 112

### Preparation of Morpholine-4-carboxylic acid [2-(3-methyl-4-oxo-2,4-dihydro-pyrazolo[4,3-c]quinolin-5-yl)-ethyl]-amide

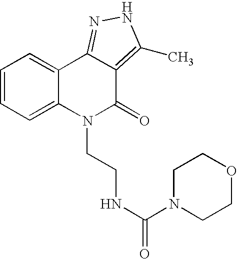

Using the appropriate reagents and in a manner similar to that exemplified in Example 35, the title compound was obtained as a white solid. ¹H NMR (400 MHz, DMSO-d6) δ 8.1 (1H, d, J=7.7 Hz); 7.9 (1H, s, br); 7.6 (1H, s, br); 7.3 (1H, s, br); 6.9 (1H, t, J=5.7 Hz); 4.2 (2H, m, br); 3.5 (4h, t, J=4.6 Hz); 3.3 (2H, m); 3.2 (4H, t, J=4.5 Hz); 2.6 (3H, br, s). LCMS: Method D, 20-100% , Rₜ=80.5 min, [MH+=356].

### NO EXAMPLE 113

### NO EXAMPLE 114

### NO EXAMPLE 115

### EXAMPLE 116

### Preparation of [5-(3-Amino-propyl)-3-methyl-4-methylene-4,5-dihydro-2H-pyrazolo [4,3-c]quinolin-8-yl]-butyl-amine

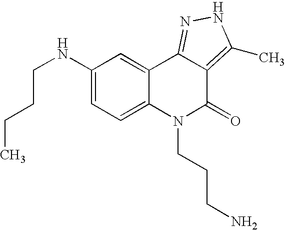

The title compound was prepared from Example 56 using the appropriate reagents and in an analogous manner to Example 120, providing a white solid. ¹H NMR (400 MHz, D₂O) δ 8.02 (1H, d, J=2.5 Hz); 7.63 (1H, d, J=9.4 Hz); 7.55 (1H, dd, J=9.1, 2.5 Hz); 4.35 (2H, t, J=6.9 Hz); 3.41 (2H, t, J=7.8 Hz); 3.01 (2H, t, J=7.5 Hz); 2.58 (3H, s); 2.07 (2H, quintet, J=7.1 Hz); 1.66 (2H, quintet, J=7.6 Hz); 1.36 (2H, apparent sextet, J=7.5 Hz); 0.85 (3H, t, J=7.4 Hz). LCMS: Method B, Rₜ=3.04 Min, m/z=326 (ES−, M−H), 350 (ES+, M+Na), 328 (ES+, M+H), 311 (ES+, M-NH₂).

### EXAMPLE 117

### Preparation of 2-Amino-3-(4-chloro-phenyl)-N-[3-(3-methyl-4-oxo-2,4-dihydro-pyrazolo[4,3-c]quinolin-5-yl)-propyl]-propionamide

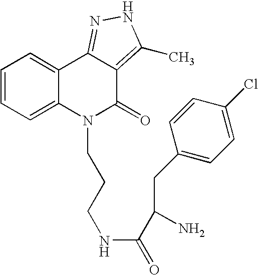

Using the appropriate reagent and in a manner similar to that exemplified in Example 104, the title compound was obtained as a white solid. ¹H NMR (400 MHz, DMSO-d6) δ 8.58 (1H, t, J=5.8 Hz); 8.4-8.2 (2H, m); 8.13 (1H, d, J=7.1 Hz); 7.55 (2H, m); 7.34 (2H, d, J=8.4 Hz); 7.31 (1H, m); 7.27 (2H, d, J=8.4 Hz); 4.23 (1H, m); 4.17 (1H, m); 3.97 (1H, m); 3.23 (1H, m); 3.16 (1H, m); 3.07 (1H, dd, J=13.5, 6.4 Hz); 2.99 )1H, dd, J=13.8, 7.6 Hz); 2.57 (3H, s); 1.71 (2H, m). LCMS, Method C, 20-100% B, Rₜ=10.11 min, MH+=438.19; MH-=436.29.

### EXAMPLE 118

### Preparation of 8-Amino-5-(3-amino-propyl)-3-methyl-2,5-dihydro-pyrazolo[4,3-c]quinolin-4-one

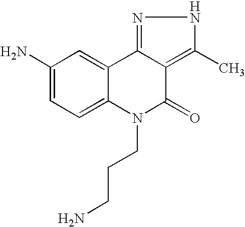

### Step 1: Preparation of {3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester

A suspension of {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester (Example 55, step 5) (0.427 mmol, 207 mg) and Pt/C (20% wt, 42 mg) in EtOH/THF (⅗ ml) was subjected to H₂ atmosphere for 12 Hours. Upon completion, the mixture was concentrated under vacuum and the residue purified by flash column chromatography, to afford the title compound {3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester as a white solid (147 mg, 76%). ¹H NMR (400 MHz, CDCl₃) δ 7.59 (d, 1H, J=2.4); 7.08 (d, 1H, J=9.14); 6.88 (dₐₚₚₐᵣₑₙₜ, 1H Broad, J=8.7); 5.52 (tapp, broad); 5.42 (dd, 1H, J=9.7, 2.03); 4.23 (t, 2H, J=6.4); 4.02 (dₐₚₚₐᵣₑₙₜ, 1H, J=11); 3.67-3.59 (m, 1H); 3.06-3.02 (m, 2H); 2.76 (s, 3H); 2.50-2.47 (m, 1H); 2.14-2.10 (m, 1H); 1.97-1.92 (m, 1H); 1.84-1.78 (m, 2H); 1.70-1.53 (m, 4H); 1.38 (s, 9H).

### Step 2

{3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester (50 mg) was treated with a 1.25M solution of HCl in MeOH (6 ml) for 4 hours. Upon completion of the reaction the reaction mixture was concentrated under vacuum and the residue successively washed with Et₂O (3 ml), MeOH (0.5 ml) and dried to afford the title compound 8-Amino-5-(3-amino-propyl)-3-methyl-2,5-dihydro-pyrazolo[4,3-c]quinolin-4-one as a white solid (33 mg). ¹H NMR (400 MHz, DMSO-d6) δ 10.5 (s, 1H, broad); 8.05 (d, 1H, J=2.56); 7.90-7.86 (2s, 2H, Broad); 7.66 (d, 1H, J=9.1); 7.47 (dd, 1H, J=9.1, 2.5); 4.25 (t, 2H, J=6.8); 2.82-2.77 (m, 2H); 2.52 (s, 3H); 1.90-1.82 (m, 2H). LCMS: Method D, 5-100% , Rₜ=7.41 Min, MH+=272, M-NH₃=255 (50%).

### EXAMPLE 119

### Preparation of [3-(3-Methyl-4-oxo-2,4-dihydro-pyrazolo[4,3-c]quinolin-5-yl)-propyl]-carbamic acid 2-bromo-benzyl ester

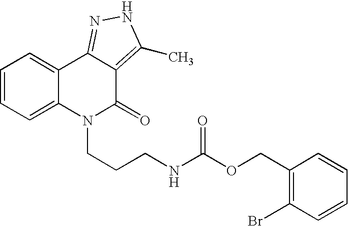

Using the appropriate reagent and in a manner similar to that exemplified in Example 35, the title compound was obtained as a white solid. ¹H NMR (400 MHz, DMSO-d6) δ 8.23 (1H, d, J=7.86 Hz); 7.76 (1H, d, J=8.05 Hz); 7.67-7.37 (7H, d, m); 5.19 (2H, s); 4.38 (2H, t, J=7.13 Hz); 3.28-3.24 (2H, m); 2.69 (3H, s); 1.94-1.86 (2H, m). LCMS, Method D, 40-100% B, Rₜ=10.61 min, MH+=469.2 (m); MH−=467.22/471.26.

### EXAMPLE 120

### Preparation of 3-(3-Methyl-4-methylene-8-pyrrolidin-1-yl-2,4-dihydro-pyrazolo[4,3-c]quinolin-5-yl)-propylamine

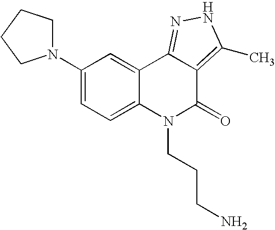

The title compound was prepared from Example 56 by the following procedure.

### Step 1: Peparation of {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester

A solution of [3-(8-bromo-3-methyl-4-oxo-1,4-dihydro-pyrazolo[4,3-c]quinolin-5-yl)-propyl]-carbamic acid tert-butyl ester (0.530 g, 1.22 mmol) in DMF (13 mL) is treated with 3,4-dihydro-2H-pyran (0.450 mL, 4.9 mmol) and para-toluenesulfonic acid (0.023 g, 0.12 mmol) and the solution stirred at room temperature. After 18 h the solution was partitioned between water and DCM and the aqueous extracted twice with DCM. The combined organics were washed with water 3 times and brine, dried over Na₂SO₄, filtered and concentrated. The crude product was purified by silica gel chromatography (1:1 EtOAc/isohexane) providing a white solid (0.533 g, 84% yield). ¹H NMR (400 MHz, CDCl₃) δ 8.34 (1H, d, J=2.4 Hz); 7.47 (1H, dd, J=9.1, 2.5 Hz); 7.11 (1H, d, J=9.1 Hz); 5.45 (1H, dd, J=9.5, 2.7 Hz); 5.37 (1H, br s); 4.24 (2H, t, J=6.4 Hz); 4.02 (1H, m); 3.65 (1H, m); 3.42 (1H, d, J=4.7 Hz); 3.06 (2H, q, J=6.1 Hz); 2.77 (3H, s); 2.53 (1H, m); 2.15 (1H, m); 1.95 (1H, m); 1.83 (2H, quintet, J=6.3 Hz); 1.70 (1H, m); 1.59 (1H, m); 1.38 (9H, s).

### Step 2: Preparation of {3-[3-Methyl-4-oxo-8-pyrrolidin-1-yl-2-(tetrahydro-pyran-2-yl)-2,4-dihydro-pyrazolo[4,3-c]quinolin-5-yl]-propyl}-carbamic acid tert-butyl-ester

An oven dried Schlenk tube is cooled under vacuum, refilled with nitrogen and charged with {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester (0.104 g, 0.20 mmol), tris(dibenzylideneacetone)dipalladium (0.0092 g, 0.010 mmol, 10 mol % Pd), 2-dicyclohexylphosphino-2′-(N,N-dimethylamino)biphenyl (0.0092 g, 0.024 mmol, 12 mol %) and sodium tert-butoxide (0.096 g, 1.0 mmol). The flask is evacuated and refilled with nitrogen three times before syringe addition of a solution of pyrollidine (0.050 mL, 0.6 mmol,) in dry 1,4-dioxan (6 mL). The orange solution is heated to 90° C. and after 30 minutes at 90° C. allowed to cool before removal of solvent in vacuo. The crude residue is purified by silica gel chromatography using 1:1 EtOAc:isohexane as the eluant. The desired product is obtained as a white solid (0.068 g, 67%). ¹H NMR (CDCl₃, 400 MHz) δ 7.42 (1H, d, J=2.7 Hz); 7.21 (1H, d, J=9.1 Hz); 6.76 (1H, dd, J=9.0, 2.8 Hz); 5.68 (1H,m); 5.52 (1H, dd, J=9.9, 2.6 Hz); 4.33 (2H, t, J=6.1 Hz); 4.14 (1H, d, J=11.5 Hz); 3.73 (1H, td, J=11.2, 2.4 Hz); 3.38 (4H, m); 3.12 (2H, q, J=6.0 Hz); 2.86 (3H, s); 2.65 (1H, m); 2.20 (1H, m); 2.05 (4H, m); 2.02 (1H, m); 1.92 (2H, quintet, J=6.2 Hz); 1.78 (2H, m); 1.65 (1H, m); 1.46 (9H, s).

### Step 3

{3-[3-methyl-4-oxo-8-pyrrolidin-1-yl-2-(tetrahydro-pyran-2-yl)-2,4-dihydro-pyrazolo[4,3-c]quinolin-5-yl]-propyl}-carbamic acid tert-butyl ester (0.030 g, 0.059 mmol) is dissolved in 1:1 TFA:water (4 mL) and the solution stirred for 15 minutes before removal of solvent in vacuo. Residual TFA is removed by evaporation of the residue from methanol 5 times, before evaporation from 1.25 M HCl/MeOH and treatment of the residue with 1.25 M HCl/MeOH (5 mL) over night. The solvent is removed in vacuo and the residue evaporated from methanol 3 times, then from methanol/ethyl acetate to give the hydrochloride salt of 3-(3-Methyl-4-methylene-8-pyrrolidin-1-yl-2,4-dihydro-pyrazolo[4,3-c]quinolin-5-yl)-propylamine as a white powder (0.026 g). ¹H NMR (400 MHz, D₂O) δ 7.51 (1H, br s); 7.24 (2H, br s); 3.94 (2H, t, J=6.4 Hz); 3.43 (4H, m); 2.72 (2H, t, J=7.4 Hz); 2.23 (3H, s); 1.98 (4H, m); 1.76 (2H, quintet, J=7.1 Hz). LCMS: Method A, Rₜ=6.22, Method D, 40-100% B, 6.22 min, [MH+=326].

### EXAMPLE 121

### Preparation of 5-(3-Amino-propyl)-3-methyl-8-(4-methyl-piperazin-1-yl)-2,5-dihydro-pyrazolo[4,3-c]quinolin-4-one

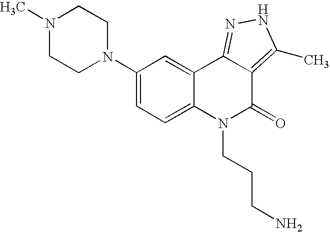

Starting from Example 56 and the appropriate reagents, and in a manner similar to that exemplified in Example 120, the title compound was obtained as a white solid. ¹H NMR (400 MHz, D₂O) δ 7.2 (2H, m); 7.16 (1H, dd, J=9.3, 2.4 Hz); 4.04 (2H, t, J=6.5 Hz); 3.75 (2H, d, J=13.7 Hz); 3.59 (2H, d, J=12.1 Hz); 3.21 (2H, t, J=6.9 Hz); 3.05 (2H, t, J=12.2 Hz); 2.87 (3H, s); 2.84 (2H, t, J=7.6 Hz); 2.37 (3H, s); 1.88 (2H, quintet, J=6.9 Hz). LCMS: Method C, 5 to 60% B, Rₜ=3.67 min, 355 (ES+, M+H).

### EXAMPLE 122

### Preparation of 2-Amino-N-[3-(3-methyl-4-oxo-2,4-dihydro-pyrazolo[4,3-c]quinolin-5-yl)-propyl]-acetamide

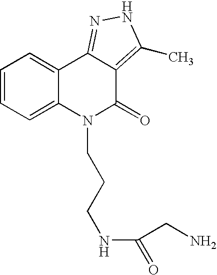

Using the appropriate reagents and in a manner similar to that exemplified in Example 104, the title compound was obtained as a white solid. ¹H NMR (400 MHz, DMSO-d6) δ 8.53 (1H, t, J=5.5 Hz); 8.15 (1H, d, J=7.6 Hz); 8.09 (3H, br s); 7.60 (2H, m); 7.32 (1H, m); 4.29 (2H, t, J=7.8 Hz); 3.56 (2H, m); 3.27 (2H, q, J=6.6 Hz); 2.58 (3H, s); 1.80 (2H, quintet, J=7.1 Hz). LCMS, Method D, 20-100% B, Rₜ=6.95 min, MH+=314.36; MH−=312.39.

### EXAMPLE 123

### Preparation of 2,6-Diamino-hexanoic acid [3-(3-methyl-4-oxo-2,4-dihydro-pyrazolo[4,3-c]quinolin-5-yl)-propyl]-amide

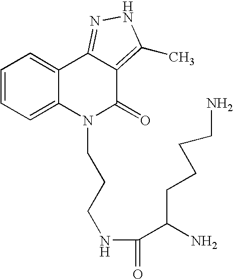

Using the appropriate reagents and in a manner similar to that exemplified in Example 104, the title compound was obtained as a white solid. ¹H NMR (400 MHz, DMSO-d6) δ 8.8 (1H, t, J=5.7 Hz); 8.30 (2H, d, br, J=4.2 Hz); 8.16 (1H, dd, J=7.7, 1.1 Hz); 8.00 (2H, s, br); 7.58-7.64 (2H. M, br); 7.31 (1H, t, J=7.3); 4.3 (2H, quartet, J=6.8 Hz); 3.78 (1H, m); 3.28-3.40 (1H, m); 3.20-3.28 (1 h, m); 2.60 (3H, m, br); 2.57 (3H, s, br); 1.72-1.84 (3H, m); 1.56-1.62 (2H, m); 1.40 (2H, quintet. J=7.5 Hz). LCMS, Method D, 20-100% B, Rₜ=6.19 min, MH+=385.42; MH−=383.44.

### EXAMPLE 124

### Preparation of 3-Amino-N-[3-(3-methyl-4-oxo-2,4-dihydro-pyrazolo[4,3-c]quinolin-5-yl)-propyl]-propionamide

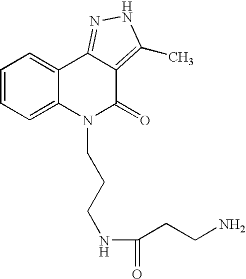

Using the appropriate reagents and in a manner similar to that exemplified in Example 104, the title compound was obtained as a white solid. ¹H NMR (400 MHz, DMSO-d6) δ 8.28 (1H, t, J=5.3 Hz); 8.17 (1H, d, J=7.50 Hz); 7.87 (2H, s, broad); 7.6 (2H, m); 7.36-7.32 (1H, m); 4.31 (2H, t, J=7.3 Hz); 3.23 (2H, dd, J=6.58, 12.80 Hz); 3.06-2.99 (2H, m); 2.6 (3H, s); 2.53 (2H, m); 1.84-1.77 (2H, m). LCMS, Method D, 20-100% B, Rₜ=6.73 min, MH+=328.41; MH−=326.39.

### EXAMPLE 125

### Preparation of N-[3-(3-Methyl-4-oxo-2,4-dihydro-pyrazolo[4,3-c]quinolin-5-yl)-propyl]-4-trifluoromethoxy-benzamide

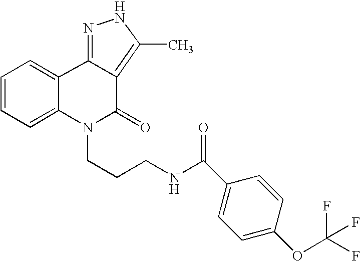

Using the appropriate reagents and in a manner similar to that exemplified in Example 35, the title compound was obtained as a white solid. ¹H NMR (400 MHz, DMSO-d6) δ 13.71 (1H, br s); 8.66 (1H, t, J=5.5 Hz); 8.12 (1H, d, J=7.5 Hz); 7.98 (2H, d, J=8.8 Hz); 7.57 (2H, m); 7.47 (2H, d, J=8.2 Hz); 7.31 (2H, t, J=7.1 Hz); 4.34 (2H, t, J=7.8 Hz); 3.40 (2H, m); 2.58 (3H, s); 1.91 (2H, quintet, J=7.2 Hz). LCMS: Method C, 40-100% B, Rₜ5.74 min, [MH+=445.29].

### EXAMPLE 126

### Biological Assays

Chk1 Expression & Purification:

Recombinant human Chk1 was expressed as a fusion protein with glutathione S-transferase at the amino-terminus (GST-Chk1) using standard baculovirus vectors and (Bac-to-Bac®) insect cell expression system purchased from GIBCO™ Invitrogen. Recombinant protein expressed in insect cells was purified using glutathione sepharose (Amersham Biotech) using standard procedures described by the manufacturer.

Chk1 Fluorescence Polarization Assays:

Chk1 kinase inhibitors were identified using fluorescence polarization to monitor kinase activity. This assay utilized 10 nM GST-Chk1 and contained 5 mM 2-(N-Morpholino)ethanesulfonic acid (MES, pH 6.5), 5 mM magnesium chloride (MgCl₂), 0.05% Tweeng-20, 1 uM adenosine 5′ triphosphate (ATP), 2 mM 1,4-Dithio-DL-threitol (DTT), 1 uM peptide substrate (Biotin-ILSRRPSYRKILND-free acid) (SEQ ID NO: 1), 10 nM peptide substrate tracer (Fluorescine-GSRRP-pS-YRKI-free acid) (pS=phosphorylated-Serine) (SEQ ID NO: 2), 60 ng anti-phospho-CREB(S133) mouse monoclonal IgG purified on Protein G sepharose from crude mouse ascites purchased from Cell Signaling Technologies (Beverly, Mass.), 4% dimethyl sulfoxide (DMSO) and 30 uM inhibitor. Reactions were incubated at room temperature for 140 minutes and terminated by addition of 25 mM EDTA (pH 8.0). Stopped reactions were incubated for 120 minutes at room temperature and fluorescence polarization values determined using a Molecular Devices/LJL Biosystems Analyst™ AD (Sunnyvale, Calif.) with standard fluorescine settings.

Additonal assays were also used to determine inhibitor potency and ability of inhibitors to compete for ATP binding site of Chk1:

Chk1 SPA Filtration Assay:

Assays (25 μL) contained 10 nM GST-Chk1, 10 mM MES, 2 mM DTT, 10 mM MgCl₂, 0.025% Tween®-20, 1 uM peptide substrate (Biotin-ILSRRPSYRKILND-free acid) (SEQ ID NO: 1), 1 uM ATP, 0.1 uCi ³³P-γ-ATP (New England Nuclear, NEN) and reacted for 90 minutes at room temperature. Reactions were terminated by adding 55 μL of phosphate buffered saline containing 50 mM EDTA, 6.9 mM ATP, 0.5 mg Scintilation proximity assay (SPA) beads (Amersham Biosciences). Peptide substrate was allowed to bind beads for 10 minutes at room temperature followed by filtration on a Packard GF/B Unifilter plate and washing with phosphate buffered saline. Dried plates were sealed with Topseal™ SEN) and ³³P incorporated to peptide substrate detected using a Packard Topcount™ scintillation counter with standard settings for ³³P.

Chk1 FlashPlate® Kinase Assay:

Assays (25 μL) contained 8.7 nM GST-Chk1, 10 mM MES, 0.1 mM ethylene glycol-bis(β-aminoethylether)-N,N,N′,N′-tetracetic acid (EGTA, pH 8.0), 2 mM DTT, 0.05% Tween 20, 3 uM peptide substrate (Biotin-ILSRRPSYRKILND-free acid) (SEQ ID NO: 1), 1 uM ATP, 0.4 uCi ³³P-γ-ATP (NEN), 4% DMSO. Reactions were incubated for 30 minutes at room temperature, terminated with 50 μL of 50 mM EDTA and 90 μL were transferred to streptavidin-coated FlashPlates® (NEN) and incubated for 1 hour at room temperature. Plates were washed with phosphate buffered salaine containing 0.01% Tween-20 and 10 mM sodium pyrophosphate. Plates were dried, sealed with Topseal™ (NEN) and amount of ³³P incorporated into the peptide substate measure using a Packard Topcount® NXT™ scintillation counter with standard settings.

The compounds of Examples 4-6, 9-22, 29, 31, 32, 34-36, 39-48, 50-53, 55-76, 78-86, 103-106, 108, 109, 116, 117, and 119-125 have IC₅₀ values less than 1 μM in this assay.

The compounds of Examples 1-3, 7, 8, 25, 30, 37, 87, 89-95, 97-102, 107, 110 and 112 have IC₅₀ values greater than 1 μM and less than 20 μM in this assay.

The compounds of Examples 23, 24 and 96 have IC₅₀ values greater than 20 μM in this assay.

Additionally, compounds 168-201, 204, 207, 210-212, 215, 233, 240, 245, 251, 313-315, 318, 351, 355-357, 359, and 361 have IC₅₀ values less than 1 μM in this assay.

Chk1 DELFIA® Kinase Assay:

Assays (25 μL) utilized 6.4 nM GST-Chk1 containing 25 mM Tris, pH 8.5, 20% glycerol, 50 mM sodium chloride (NaCl), 0.1% Surfact-Amps® 20, 1 uM peptide stubstrate (Biotin-GLYRSPSMPEN-amide) (SEQ ID NO: 3), 2 mM DTT, 4% DMSO, 12.5 uM ATP, 5 mM MgCl₂ and reacted for 30 minutes at room temperature. Reactions were terminated with 100 μL of Stop buffer containing 1% BSA, 10 mM Tris, pH 8.0, 150 mM NaCl, 100 mM EDTA. Stopped reactions (100 μL) were transferred to 96 well neutravidin plates (Pierce) to capture the biotin-peptide substrate during a 30 minute room temperature incubation. Wells were washed and reacted with 100 μL PerkinElmer Wallac Assay Buffer containing 21.5 ng/ml anti-phospho-Ser216-Cdc25c rabbit polyclonal antibody from Cell Signaling Technology (Beverly, Mass.) and 292 ng/ml europium labeled anti-rabbit-IgG for 1 hour at room temperature. Wells were washed and europium released from the bound antibody by addition of Enhancement Solution (100 μL) (PerkinElmer Wallac) and detected using a Wallac Victor2™ using standard manufacturer settings.

Chk1 DELFIA® Kinase Assay:

Assays (25 μL) utilized 2 nM GST-Chk1 containing 10 mM Tris, pH 7.5, 20% glycerol, 50 mM sodium chloride (NaCl), 0.01% Surfact-Amps® 20, 1 μM peptide stubstrate (Biotin-GLYRSPSMPEN-amide) (SEQ ID NO: 3), 01.% BSA, 2 mM DTT, 4% DMSO, 600 μM ATP, 10 MM MgCl₂ and reacted for 50 minutes at room temperature. Reactions were terminated with 100 μL of Stop buffer containing 1% BSA, 10 mM Tris, pH 8.0, 150 mM NaCl, 100 mM EDTA. Stopped reactions (100 μL) were transferred to 96 well NeutrAvidin plates (Pierce) to capture the biotin-peptide substrate during a 30 minute room temperature incubation. Wells were washed and reacted with 100 μL PerkinElmer Wallac Assay Buffer containing 21.5 ng/ml anti-phospho-Ser216-Cdc25c rabbit polyclonal antibody from Cell Signaling Technology (Beverly, Mass.) and 292 ng/ml europium labeled anti-rabbit-IgG for 1 hour at room temperature. Wells were washed and europium released from the bound antibody by addition of Enhancement Solution (100 μL) (PerkinElmer Wallac) and detected using a Perkin Elmer Wallac Envison™ 2100 multilabel reader using standard manufacturer settings.

Compounds 202, 203, 205, 206, 208, 209, 213, 214, 216-232, 234-239, 242-244, 246-250, 252, 253, 255-265, 267, 268, 270-311, 316, 320-322, 325, 327-333, 335-342, 345, 348, 349, 352-354, 357, 358, 360, 362-369 have IC₅₀ values less than 1 μM in this assay.

Compounds 266, 269, 324, 334, 343, 344, and 347 have IC₅₀ values greater than 1 μM and less than 10 μM in this assay.

### EXAMPLES 127-167

### Are intentionally omitted To Facilitate Numbering

Examples 168-369 correspond to the compound numbers from the compound table above.

### EXAMPLE 168

### Preparation of 5-(3-Amino-propyl)-7-fluoro-3-methyl-2,5-dihydro-pyrazolo[4,3-c]quinolin-4-one

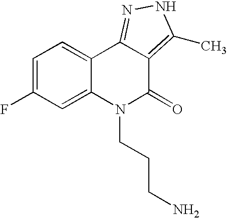

The title compound was prepared from 2-Amino-4-fluoro-benzoic acid by methods outlined in Example 55.

### EXAMPLE 169

### Preparation of 5-(3-Amino-propyl)-3-methyl-8-trifluoromethoxy-2,5-dihydro-pyrazolo[4,3-c]quinolin-4-one

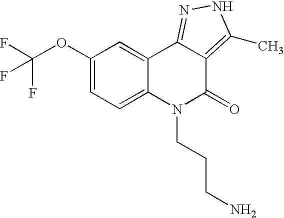

The title compound was prepared from 2-Amino-5-trifluoromethoxy-benzoic acid by methods outlined in Example 55.

### EXAMPLE 170

### Preparation of 5-(3-Amino-propyl)-8-chloro-3-cyclopropyl-2,5-dihydro-pyrazolo[4,3-c]quinolin-4-one

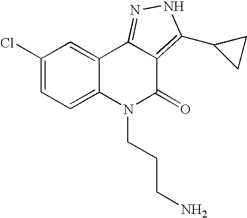

The title compound was prepared according to methods outlined in Example 49 and 50 using 3-Cyclopropyl-3-oxo-propionic acid ethyl ester as the acylating agent.

### EXAMPLE 171

### Preparation of 5-(3-Amino-propyl)-8-chloro-3-hydroxymethyl-2,5-dihydro-pyrazolo[4,3-c]quinolin-4-one

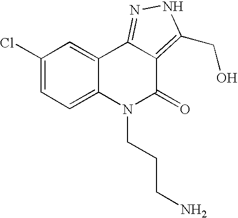

The title compound was prepared by methods outlined in Example 316.

### EXAMPLE 172

### Preparation of 5-(3-Amino-propyl)-8-chloro-3-methoxymethyl-2,5-dihydro-pyrazolo[4,3-c]quinolin-4-one

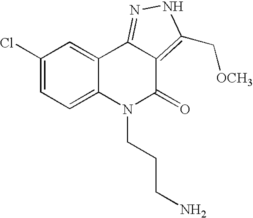

The title compound was prepared by according to methods outlined in Example 32 using 4-Methoxy-3-oxo-butyric acid methyl ester as the acylating agent.

### EXAMPLE 173

### Preparation of 8-Chloro-5-(3-dimethylamino-propyl)-3-(2-methoxy-ethyl)-2,5-dihydro-pyrazolo[4,3-c]quinolin-4-one

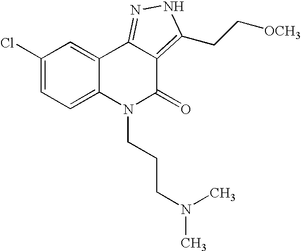

The title compound was prepared from Example 49 by methods outlined in Example 50 using (3-Chloro-propyl)-dimethyl amine as the aklyating agent.

### EXAMPLE 174

### Preparation of 8-Chloro-5-(3-dimethylamino-2,2-dimethyl-propyl)-3-methyl-2,5-dihydro-pyrazolo[4,3-c]quinolin-4-one

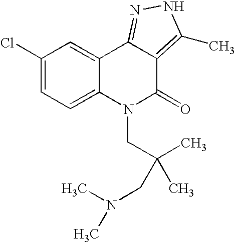

The title compound was prepared in an analogous manner to Example 16 using the bromide obtained from 3-Dimethylamino-2,2-dimethyl-propan-1-ol following procedures outlined in Example 212.

### EXAMPLE 175

### Preparation of 8-Chloro-5-(3-imidazol-1-yl-propyl)-3-methyl-2,5-dihydro-pyrazolo[4,3-c]quinolin-4-one

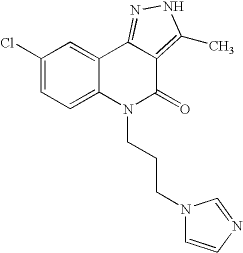

The title compound was prepared from the mesylate of Example 209, prepared as described for Example 7 (Step 2), and imidazole.

### EXAMPLE 176

### Preparation of N-[3-(8-Bromo-3-methyl-4-oxo-2,4-dihydro-pyrazolo[4,3-c]quinolin-5-yl)-propyl]-4-chloro-benzamide

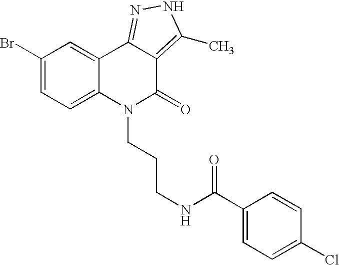

The title compound was prepared from Example 57 by methods outlined in Example 35.

### EXAMPLE 177

### Preparation of N-{3-[8-Bromo-3-(2-methoxy-ethyl)-4-oxo-2,4-dihydro-pyrazolo[4,3-c]quinolin-5-yl]-propyl}-4-chloro-benzamide

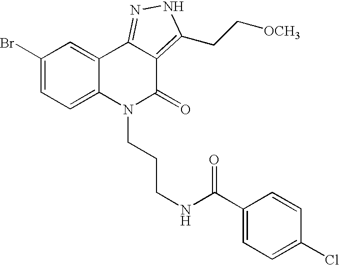

The title compound was obtained from5-(3-Amino-propyl)-8-bromo-3-(2-methoxy-ethyl)-2,5-dihydro-pyrazolo[4,3-c]quinolin-4-one prepared in a manner analogous to Example 50 after treatment with 4-Chloro benzoyl chloride as outlined in Example 35.

### EXAMPLE 178

### Preparation of N-[3-(8-Bromo-3-methyl-4-oxo-2,4-dihydro-pyrazolo[4,3-c]quinolin-5-yl)-propyl]-2-phenyl-acetamide

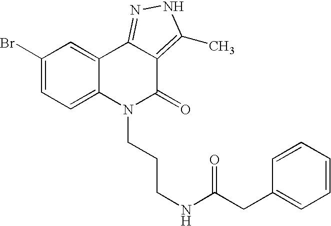

The title compound was prepared similarly to Example 177 using Phenylacetyl chloride.

### EXAMPLE 179

### Preparation of Cyclohexanecarboxylic acid [3-(8-bromo-3-methyl-4-oxo-2,4-dihydro-pyrazolo[4,3-c]quinolin-5-yl)-propyl]-amide

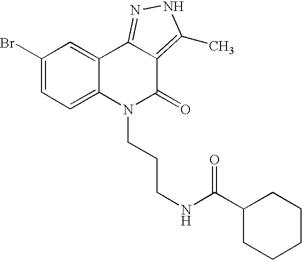

The title compound was prepared similarly to Example 177 using Cyclohexanecarbonyl chloride

### EXAMPLE 180

### Preparation of N-[3-(3-Methyl-4-oxo-2,4-dihydro-pyrazolo[4,3-c]quinolin-5-yl)-propyl]-isonicotinamide

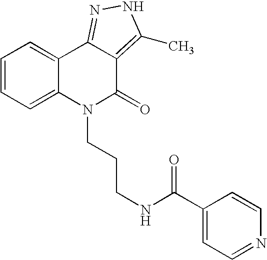

The title compound was prepared from Example 15 by methods outlined in Example 35.

### EXAMPLE 181

### Preparation of 2-Phenyl-cyclopropanecarboxylic acid [3-(3-methyl-4-oxo-2,4-dihydro-pyrazolo[4,3-c]quinolin-5-yl)-propyl]-amide

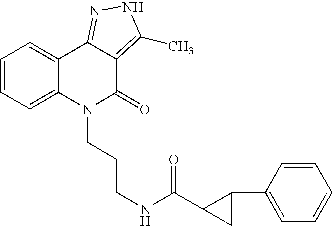

The title compound was prepared from Example 15 by methods outlined in Example 35.

### EXAMPLE 182

### Preparation of 8-Chloro-3-methyl-5-(3-morpholin-4-yl-propyl)-2,5-dihydro-pyrazolo[4,3-c]quinolin-4-one

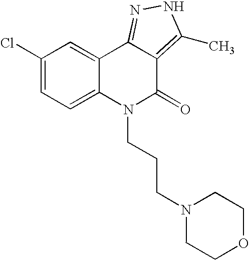

The title compound was prepared according to Example 175 using morpholine.

### EXAMPLE 183

### Preparation of 3-Amino-3-(4-chloro-phenyl)-N-[3-(3-methyl-4-oxo-2,4-dihydro-pyrazolo[4,3-c]quinolin-5-yl)-propyl]-propionamide

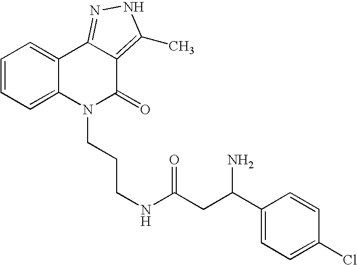

Using the appropriate reagents, the title compound was prepared from Example 15 by methods outlined in Example 104.

### EXAMPLE 184

### Preparation of 2S-Amino-3-(3,4-difluoro-phenyl)-N-[3-(3-methyl-4-oxo-2,4-dihydro-pyrazolo[4,3-c]quinolin-5-yl)-propyl]-propionamide

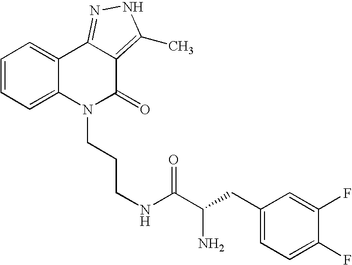

Using the appropriate reagents, the title compound was prepared similarly to Example 183.

### EXAMPLE 185

### Preparation of 2S-Amino-3-(1H-imidazol-4-yl)-N-[3-(3-methyl-4-oxo-2,4-dihydro-pyrazolo[4,3-c]quinolin-5-yl)-propyl]-propionamide

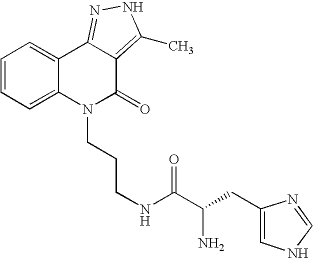

Using the appropriate reagents, the title compound was prepared similarly to Example 183.

### EXAMPLE 186

### Preparation of 2-Amino-4-methyl-pentanoic acid [3-(3-methyl-4-oxo-2,4-dihydro-pyrazolo[4,3-c]quinolin-5-yl)-propyl]-amide

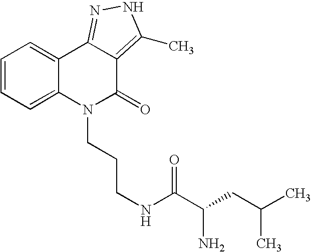

Using the appropriate reagents, the title compound was prepared similarly to Example 183.

### EXAMPLE 187

### Preparation of 1,2,3,4-Tetrahydro-isoquinoline-3-carboxylic acid [3-(3-methyl-4-oxo-2,4-dihydro-pyrazolo[4,3-c]quinolin-5-yl)-propyl]-amide

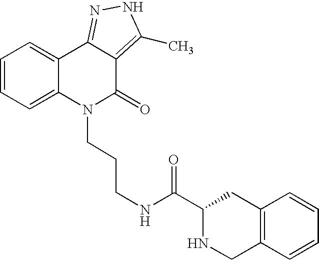

Using the appropriate reagents, the title compound was prepared similarly to Example 183.

### EXAMPLE 188

### Preparation of 2-Amino-3-cyclohexyl-N-[3-(3-methyl-4-oxo-2,4-dihydro-pyrazolo[4,3-c]quinolin-5-yl)-propyl]-propionamide

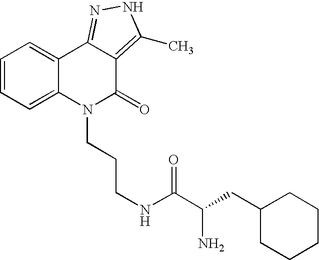

Using the appropriate reagents, the title compound was prepared similarly to Example 183.

### EXAMPLE 189

### Preparation of 2-Amino-N-[3-(3-methyl-4-oxo-2,4-dihydro-pyrazolo[4,3-c]quinolin-5-yl)-propyl]-4-phenyl-butyramide

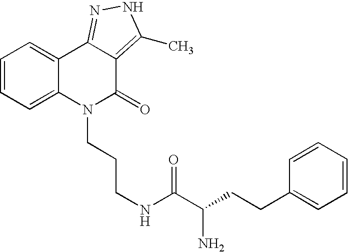

Using the appropriate reagents, the title compound was prepared similarly to Example 183.

### EXAMPLE 190

### Preparation of 1-Amino-cyclohexanecarboxylic acid [3-(3-methyl-4-oxo-2,4-dihydro-pyrazolo[4,3-c]quinolin-5-yl)-propyl]-amide

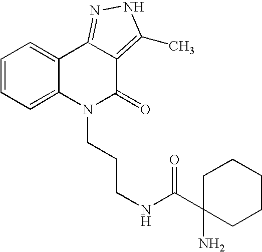

Using the appropriate reagents, the title compound was prepared similarly to Example 183.

### EXAMPLE 191

### Preparation of 2-Amino-3-(4-chloro-phenyl)-N-[3-(8-fluoro-3-methyl-4-oxo-2,4-dihydro-pyrazolo[4,3-c]quinolin-5-yl)-propyl]-propionamide

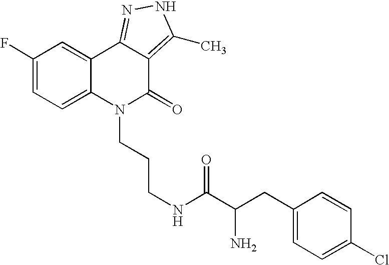

Using the appropriate reagents, the title compound was prepared from Example 203 by methods outlined in Example 104.

### EXAMPLE 192

### Preparation of 5-(3-Amino-propyl)-3-methyl-8-morpholin-4-yl-2,5-dihydro-pyrazolo[4,3-c]quinolin-4-one

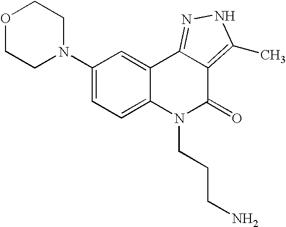

The title compound was prepared by methods outlined in Example 120.

### EXAMPLE 193

### Preparation of 5-(3-Amino-propyl)-3-methyl-8-(4-methyl-piperazin-1-yl)-2,5-dihydro-pyrazolo[4,3-c]quinolin-4-one

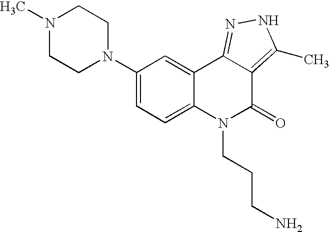

The title compound was prepared by methods outlined in Example 120.

### EXAMPLE 194

### Preparation of 5-(3-Amino-propyl)-8-pyrrolidin-1-yl-3-vinyl-2,5-dihydro-pyrazolo[4,3-c]quinolin-4-one

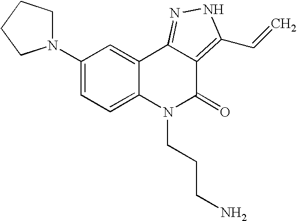

The title compound was prepared by methods outlined in Example 120.

### EXAMPLE 195

### Preparation of 4-(8-Bromo-3-methyl-4-oxo-2,4-dihydro-pyrazolo[4,3-c]quinolin-5-yl)-butyramide

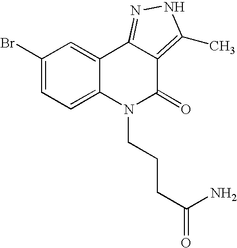

The title compound was prepared from Example 19 by methods outlined in Example 21.

### EXAMPLE 196

### Preparation of 5-(8-Bromo-3-methyl-4-oxo-2,4-dihydro-pyrazolo[4,3-c]quinolin-5-yl)-pentanoic acid methyl ester

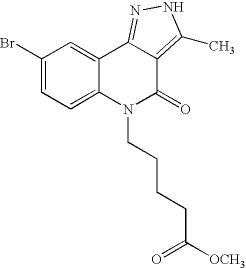

The title compound was prepared in an analogous manner to Example 17.

### EXAMPLE 197

### Preparation of 5-(8-Bromo-3-methyl-4-oxo-2,4-dihydro-pyrazolo[4,3-c]quinolin-5-yl)-pentanoic acid

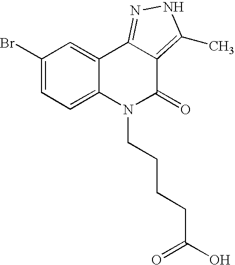

The title compound was prepared from Example 196 in an analogous manner to Example 19.

### EXAMPLE 198

### Preparation of N-(4-Chloro-benzyl)-3-(8-chloro-3-methyl-4-oxo-2,4-dihydro-pyrazolo[4,3-c]quinolin-5-yl)-propionamide

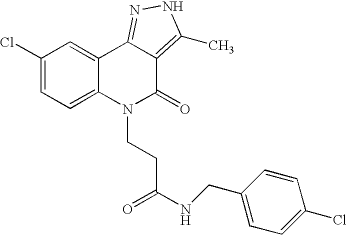

Using the appropriate reagents, the title compound was prepared by methods outlined in Example 21.

### EXAMPLE 199

### Preparation of N-(4-Chloro-benzyl)-3-(3-methyl-4-oxo-2,4-dihydro-pyrazolo[4,3-c]quinolin-5-yl)-propionamide

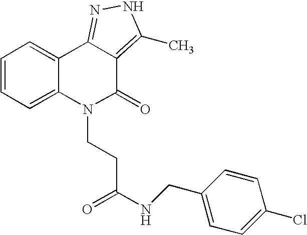

Using the appropriate reagents, the title compound was prepared by methods outlined in Example 21.

### EXAMPLE 200

### Preparation of 8-Bromo-3-methyl-5-(4-oxo-4-piperazin-1-yl-butyl)-2,5-dihydro-pyrazolo[4,3-c]quinolin-4-one

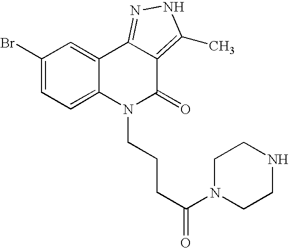

The title compound was prepared from Example 19 and mono-N-Boc piperazine using methods outlined in Example 21.

### EXAMPLE 201

### Preparation of N-[2-(3-Methyl-4-oxo-2,4-dihydro-pyrazolo[4,3-c]quinolin-5-yl)-ethyl]-guanidine

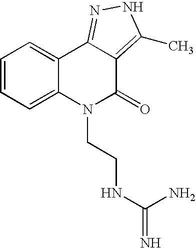

The title compound was prepared from Example 11 by methods outlined in Example 253.

### EXAMPLE 202

### Preparation of 5-(3-Amino-2-methyl-propyl)-8-fluoro-3-methyl-2,5-dihydro-pyrazolo[4,3-c]quinolin-4-one

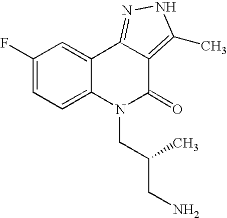

### Step 1: Preparation of 2-Amino-5-fluoro-benzoic acid methyl ester

To a solution of 5-fluoro-2-nitro-benzoic acid methyl ester (24.8 g, 125 mmol) in CH₃OH was added 10% by weight Pd on activated carbon (2.58 g) under nitrogenous atmosphere. The vessel was then purged with and stirred under 1 atmosphere H₂ for 5 d. Filtration through celite and silica gel chromatography eluting with a gradient of 0 to 30% ethyl acetate in hexanes afforded 19.7 g (88%) of the title compound as a yellow oil. ¹H NMR (300 MHz, CDCl₃) δ 7.53 (dd, J=3.1, 9.7 Hz, 1H), 7.03 (ddd, J=3.1, 7.8, 9.0 Hz, 1H), 6.62 (dd, J=4.5, 9.0 Hz, 1H), 5.57 (br s, 2H), 3.87 (s, 3H); ¹⁹F NMR (282 MHz, CDCl₃) ppm −128.80 (ddd, J=4.9, 8.0, 9.7 Hz); LC/MS: FA standard Rₜ=1.60 min, EI⁺ 170.07.

### Step 2: Preparation of 5-Fluoro-2-(3-oxo-butyrylamino)-benzoic acid methyl ester

A solution of 2-amino-5-fluoro-benzoic acid methyl ester (17.5 g, 103 mmol) and 3-oxo-butyric acid methyl ester (22.3 mL, 207 mmol) in toluene was heated to reflux using a Soxhlet extractor filled with 3 angstrom molecular sieves. After 20 h, the molecular sieves were replaced and more 3-oxo-butyric acid methyl ester (11.2 mL, 104 mmol) was added, and the solution refluxed 1d. Concentration in vacuo afforded 25.5 g (97%) of the title compound as an off-white powder. ¹H NMR (300 MHz, DMSO-d₆) δ 10.5 (s, 1H), 8.07 (dd, J=5.2, 9.1 Hz, 1H), 7.62 (dd, J=3.1, 9.2 Hz, 1H), 7.49 (ddd, J=3.2, 8.1, 9.1 Hz, 1H), 3.84 (s, 3H), 3.64 (s, 2H), 2.22 (s, 3H); ¹⁹F NMR (282 MHz, DMSO-d₆) ppm −118.34 to 118.26 (m); LC/MS: AA standard Rₜ=1.52 min, EI⁺ 254.19.

### Step 3: Preparation of 3-Acetyl-6-fluoro-4-hydroxy-1H-quinolin-2-one

To a suspension of 5-fluoro-2-(3-oxo-butyrylamino)-benzoic acid methyl ester (23.5 g, 92.8 mmol) in CH₃OH was added NaOCH₃ solution in CH₃OH (40.1 mL, 186 mmol) dropwise via syringe. The mixture was refluxed for 1 h, diluted with 1.0N HCl solution (190 mL) and filtered. The resulting solid was washed with H₂O (2×) and Et₂O (2×) and dried under high vacuum. Four additional crops were collected by concentration of the filtrate, filtration and washing of the solid. The combined crops gave 20.5 g (100%) ofthe title compound as a white solid. ¹H NMR (300 MHz, DMSO-d₆) δ 11.3 (br s, 1H), 7.60 (dd, J=2.8, 9.5 Hz, 1H), 7.35 (ddd, J=2.8, 8.4, 8.5 Hz, 1H), 7.16 (dd, J=4.6, 8.8 Hz, 1H), 2.53 (s, 3H); ¹⁹F NMR (282 MHz, DMSO-d₆) ppm −123.23 to −122.69 (m); LC/MS: AA standard Rₜ=1.46 min, EI⁺ 222.09.

### Step 4: Preparation of 8-Fluoro-3-methyl-2,5-dihydro-pyrazolo[4,3-c]quinolin-4-one

To a suspension of 3-acetyl-6-fluoro-4-hydroxy-1H-quinolin-2-one (21.8 g, 98.6 mmol) in DMF was added hydrazine hydrate (14.5 mL, 298 mmol) and the mixture was heated to reflux for 3 h. The solution was carefully quenched with 1.0N HCl solution (350 mL), stirred for 1 h and filtered. The filtered material was washed with H₂O (2×) and Et₂O (2×) before being dried under high vacuum to afford 19.0 g (76%) of the title compound as a yellow solid. ¹H NMR (300 MHz, DMSO-d₆) δ 13.7 (s, 1H), 11.3 (s, 0.5H), 11.0 (s, 0.5H) [tautomers], 7.82 (d, J=8.5 Hz, 0.5H), 7.69 (d, J=8.1 Hz, 0.5H) [tautomers], 7.22-7.53 (m, 2H), 2.62 (s, 3H); ¹⁹F NMR (282 MHz, DMSO-d₆) −121.20 to −121.04 (m, 0.5F), −121.50 to −121.32 (m, 0.5F) [tautomers]; LC/MS: AA standard Rₜ=1.09 min, EI⁺ 218.18.

### Step 5: Preparation of 8-Fluoro-3-methyl-2-(tetrahydro-pyran-2-yl)-2,5-dihydro-pyrazolo[4,3-c]quinolin-4-one

A mixture of 8-fluoro-3-methyl-2,5-dihydro-pyrazolo[4,3-c]quinolin-4-one hydrochloride (17.0 g, 67.0 mmol), 3,4-dihydro-2H-pyran (24.5 mL, 270 mmol) and p-toluenesulfonic acid (1.27 g, 6.68 mmol) was heated to 70° C. for 18 h. Dilution of the mixture with Et₂O followed by filtration afforded 22.1 g (100%) of the title compound as a white powder. ¹H NMR (300 MHz, DMSO-d₆) δ 11.1 (s, 1H), 7.63-7.72 (m, 1H), 7.24-7.38 (m, 2H), 5.62-5.71 (m, 1H), 3.89-3.99 (m, 1H), 3.66-3.79 (m, 1H), 2.33-2.50 (m, 1H), 1.91-2.13 (m, 2H), 1.50-1.83 (m, 3H); ¹⁹F NMR (282 MHz, DMSO-d₆) −121.18 to −121.04 (m); LC/MS: AA standard Rₜ=1.61 min, EI⁺ 302.27.

### Step 6: Preparation of (R)-{3-[8-Fluoro-3-methyl-4-oxo-2-(tetrahydro-pyran-2-yl)-2,4-dihydro-pyrazolo[4,3-c]quinolin-5-yl]-2-methyl-propyl}-carbamic acid tert-butyl ester

A mixture of 8-Fluoro-3-methyl-2-(tetrahydro-pyran-2-yl)-2,5-dihydro-pyrazolo[4,3-c]quinolin-4-one (215 mg, 0.714 mmol) and Cs₂CO₃ (1.16 g, 3.56 mmol) in DMF was stirred 10 min before (R)-(3-Bromo-2-methyl-propyl)-carbamic acid tert-butyl ester (273 mg, 1.78 mmol) (prepared as in Example 212, Step 1 and 2) in DMF was added. After 18 h, the mixture was diluted with H₂O, extracted with ethyl acetate (3×), dried over MgSO₄, filtered and concentrated in vacuo. Chromatography eluting with 0 to 50% ethyl acetate in hexanes afforded 200 mg (59%) of the product as a white solid. ¹H NMR (300 MHz, DMSO-d₆) δ 7.80 (dd, J=3.0, 8.6 Hz, 1H), 7.5 (dd, J=4.4, 9.3 Hz, 1H), 7.37 (ddd, J=2.8, 8.9, 9.0 Hz, 1H), 6.79-6.88 (m, 1H), 5.68 (dd, J=1.6, 9.5 Hz, 1H), 4.13 (br s, 2H), 3.89-3.99 (m, 1H), 3.66-3.78 (m, 1H), 2.78-3.02 (m, 2H), 2.75 (s, 3H), 1.91-2.15 (m, 3H), 1.52-1.82 (m, 3H), 1.36 (s, 9H), 1.13-1.25 (m, 1H), 0.76-0.92 (m, 3H); ¹⁹F NMR (282 MHz, DMSO-d₆) −121.42 (ddd, J=3.6, 3.6, 11.8 Hz); LC/MS: AA standard Rₜ=2.24 min, EI⁺ 473.31.

### Step 7: Preparation of (R)-5-(3-Amino-2-methyl-propyl)-8-fluoro-3-methyl-2,5-dihydro-pyrazolo[4,3-c]quinolin-4-one hydrochloride

To a solution of (R)-{3-[8-Fluoro-3-methyl-4-oxo-2-(tetrahydro-pyran-2-yl)-2,4-dihydro-pyrazolo[4,3-c]quinolin-5-yl]-2-methyl-propyl}-carbamic acid tert-butyl ester (186 mg, 0.394 mmol) in 10:1 CH₂Cl₂:CH₃OH was added 4.OM HCl in 1,4-dioxane (1.00 mL, 4.00 mmol) and the reaction was stirred for 18 h. Concentration in vacuo afforded 128 mg (100%) of the title compound as a yellow solid. ¹H NMR (300 MHz, DMSO-d₆) d 7.92-8.08 (m, 4H), 7.65 (dd, J=4.3, 9.3 Hz, 1H), 7.42 (ddd, J=2.7, 8.8, 9.2 Hz, 1H), 4.05-4.43 (m, 2H), 2.64-2.90 (m, 2H), 2.58 (s, 3H), 2.23-2.40 (m, 1H), 1.01 (d, J=6.5 Hz, 3H) ppm; ¹⁹F NMR (282 MHz, DMSO-d₆) −121.33 (ddd, J=4.3, 8.3, 8.4 Hz); LC/MS: AA standard Rₜ=1.02 min, EI⁺ 289.15.

### EXAMPLE 203

### Preparation of 5-(3-Amino-propyl)-8-fluoro-3-methyl-2,5-dihydro-pyrazolo[4,3-c]quinolin-4-one

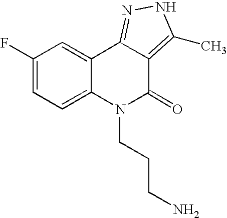

Prepared from the appropriate reagents by an analogous procedure to Example 202.

¹H NMR (acetate salt) (300 MHz, DMSO-d₆) δ 7.89 (dd, J=3.0, 8.8 Hz, 1H), 7.66 (dd, J=4.5, 9.4 Hz, 1H), 7.43 (ddd, J=2.9, 8.6, 9.0 Hz, 1H), 4.29 (t, J=5.9 Hz, 2H), 2.66 (t, J=6.8 Hz, 2H), 2.58 (s, 3H), 1.83 (s, 3H), 1.68-1.80 (m, 2H); ¹⁹F NMR (282 MHz, DMSO-d₆) −121.67 (ddd, J=4.5, 8.4, 8.6 Hz, 1H) ppm; LC/MS: AA standard Rₜ=0.99 min, EI⁺ 275.15.

### EXAMPLE 204

### Preparation of 8-Fluoro-5-(3-hydroxy-propyl)-3-methyl-2,5-dihydro-pyrazolo[4,3-c]quinolin-4-one

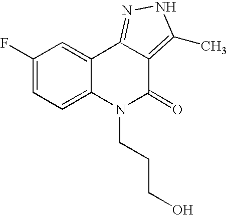

Alkylated with 3-Bromopropanol in a manner similar to Example 202. ¹H NMR (300 MHz, DMSO-d₆) δ 7.91 (dd, J=2.8, 8.8 Hz, 1H), 7.62 (dd, J=4.5, 9.3 Hz, 1H), 7.39-7.49 (m, 1H), 4.28 (t, J=7.3 Hz, 2H), 3.52 (t, J=6.0 Hz, 2H), 2.57 (s, 3H), 1.69-1.82 (m, 2H); ¹⁹F NMR (282 MHz, DMSO-d₆) −121.72 (ddd, J=4.6, 8.3, 8.4 Hz) ppm; LC/MS: AA standard Rₜ=1.16 min, EI⁺ 276.15.

### EXAMPLE 205

### Preparation of 5-(3-Amino-propyl)-8-fluoro-3-(2-methoxy-ethyl)-2,5-dihydro-pyrazolo[4,3-c]quinolin-4-one

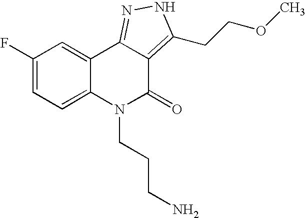

Prepared from the appropriate reagents by an analogous procedure to Example 202, Steps 2-7, using 5-methoxy-3-oxo-pentanoic acid methyl ester as the acylation component in Step 2. ¹H NMR (300 MHz, DMSO-d₆) δ 7.82-8.02 (m, 3H), 7.71 (dd, J=4.4, 9.4 Hz, 1H), 7.47 (ddd, J=2.9, 8.8, 9.1 Hz, 1H), 4.34 (dd, J=6.9, 6.9 Hz, 2H), 3.71 (dd, J=6.9, 6.9 Hz, 2H), 3.25 (s, 3H), 3.23 (dd, J=6.9, 6.9 Hz, 2H), 2.81-2.94 (m, 2H), 1.87-2.00 (m, 2H) ppm; ¹⁹F NMR (282 MHz, DMSO-d₆) −121.26 to −121.12 (m); LC/MS: AA standard Rₜ=1.06 min, EI⁺ 319.10.

### EXAMPLE 206

### Preparation of 5-(3-Amino-propyl)-8-chloro-3-(2-methoxyethyl)-2,5-dihydro-pyrazolo[4,3-c]quinolin-4-one

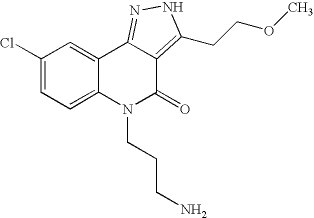

Prepared from the appropriate reagents by an analogous procedure to Example 202, Steps 2-7, using 2-amino-5-chloro-benzoic acid methyl ester and 5-methoxy-3-oxo-pentanoic acid methyl ester. ¹H NMR formate salt (300 MHz, DMSO-d₆) δ 8.39 (s, 1H), 8.20 (d, J=2.3 Hz, 1H), 7.56-7.71 (m, 2H), 4.32 (t, J=6.9 Hz, 2H), 3.71 (t, J=6.9 Hz, 2H), 3.25 (s, 3H), 3.23 (t, J=7.2 Hz, 2H), 2.84 (t, J=7.3 Hz, 2H), 1.90 (quintet, J=6.9 Hz, 2H) ppm; LC/MS: AA standard Rₜ=1.08 min, EI⁺ 335.20.

### EXAMPLE 207

### Preparation of 5-(3-Amino-propyl)-3-(2-methoxy-ethyl)-2,5-dihydro-pyrazolo[4,3-c]quinolin-4-one

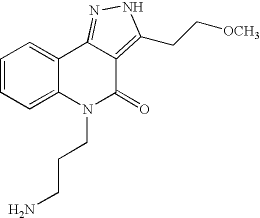

The title compound was prepared similarly to Example 206.

LCMS: Method FA, Rₜ=0.81 min, [MH⁺=301.2]. ¹H NMR 300 MHz (CD₃OD) δ 8.15 (d, 1H), 7.63-7.67 (m, 2H), 7.34-7.41 (m, 1H), 4.48 (t, 2H), 3.81 (t, 2H), 3.35 (s, 3H), 2.96-3.08 (m, 4H), 2.10-2.22 (m, 2H).

### EXAMPLE 208

### Preparation of 5-(3-Amino-propyl)-8-iodo-3-methyl-2,5-dihydro-pyrazolo[4,3-c]quinolin-4-one

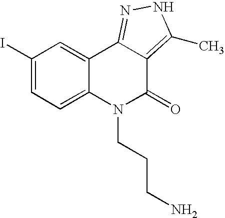

### Step 1: Preparation of 5-Iodoisatoic Anhydride

To a solution of 2-amino-5-iodobenzoic acid (50.09 g, 190.4 mmol) in 800 mL anhydrous THF at room temperature was added triphosgene (19.1 g, 64.4 mmol). The solution was stirred at room temperature for 6 hours, then stored at 0° C. for 16 hours. The precipitate was filtered and washed with diethyl ether to give 40.32 g product. The filtrate was then concentrated and the residue was triturated with THF/Ether (1:1) then filtered and washed with ether to give and additional 9.91 g product. The overall yield was 50.23 g.

### Step 2: Preparation of Methyl 2-amino-5-iodobenzoate

To a suspension of 5-iodoisatoic anhydride (50.23 g, 173.8 mmol) in 800 mL anhydrous methanol at room temperature was added 4-dimethylaminopyridine (1.97 g, 16.2 mmol). The mixture was then stirred at 80° C. for 4 hours, then cooled to room temperature and the solvent was evaporated in vacuo. The residue was partitioned between ethyl acetate and 0.1 N HCl. The layers were separated and the organic phase was then washed with 0.1 N HCl two more times, followed by brine, then dried over sodium sulfate and concentrated in vacuo to give 47.06 g product as an off-white solid.

### Step 3: Preparation of 5-(3-Amino-propyl)-8-iodo-3-methyl-2,5-dihydro-pyrazolo[4,3-c]quinolin-4-one

The title compound was prepared as described in Example 202. LCMS: Method FA, Rₜ1.05 min, [MH⁺=383.10]. ¹H NMR (300 MHz, D₂O) δ 7.74-7.77 (m, 1H), 7.66 (d, 2H), 7.00 (d, 1H), 4.11 (t, 2H), 3.03 (t, 2H), 2.54 (s, 3H), 1.94-2.08 (m, 2H).

### EXAMPLE 209

### Preparation of 8-Chloro-5-(3-hydroxy-propyl)-3-methyl-2,5-dihydro-pyrazolo[4,3-c]quinolin-4-one

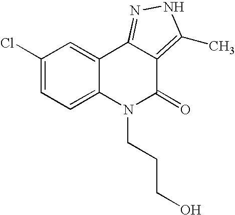

The title compound was prepared using the appropriate reagents by an analogous procedure to Example 204. ¹H NMR (300 MHz, DMSO-d₆) δ 8.19 (s, 1H), 7.64 (s, 2H), 4.31 (t, 2H), 3.55 (t, 2H), 2.61 (s, 3H), 1.73-1.84 (m, 2H).

### EXAMPLE 210

### Preparation of 5-But-3-enyl-8-chloro-3-methyl-2,5-dihydro-pyrazolo[4,3-c]quinolin-4-one

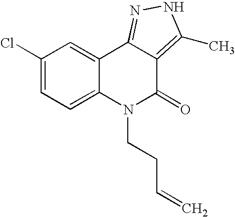

The title compound was prepared similarly to Example 204 using 4-bromobutene as the alkylating agent. ¹H NMR (300 MHz, DMSO-d₆) δ 8.18 (s, 1H), 7.63 (d, 2H), 5.86-6.01 (m, 1H), 5.03-5.14 (m, 2H), 4.34 (t, 2H), 2.61 (s, 3H), 2.37-2.45 (m, 2H).

### EXAMPLE 211

### Preparation of 5-(3-Amino-2,2-dimethyl-propyl)-8-chloro-3-methyl 2,5-dihydro-pyrazolo[4,3-c]quinolin-4-one

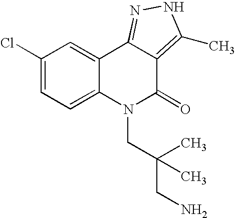

The title compound was prepared from the appropriate reagents by an analogous procedure to Example 202.

The necessary alkyation agent was prepared from 3-amino-2,2-dimethyl-propan-1-ol.

### Step 1: Preparation of (3-Hydroxy-2,2-dimethyl-propyl)-carbamic acid tert-butyl ester

3-Amino-2,2-dimethyl-propan-1-ol was Boc protected using conditions outlined in Example 212, Step 1. ¹H NMR 300 MHz (CDCl₃) δ 3.79 (t, 1H), 3.19 (d, 2H), 2.95 (d, 2H), 1.40 (s, 9H), 0.80 (s, 6H).

### Step 2: Toluene-4-sulfonic acid 3-tert-butoxycarbonylamino-2,2-dimethyl-propyl ester

To a solution of (3-Hydroxy-2,2-dimethyl-propyl)-carbamic acid tert-butyl ester (0.50 g, 2.5 mmol) and pyridine (0.78 g, 9.85 mmol) in dichloromethane at room temperature was added 4-methyl-benzenesulfonyl chloride (0.57 g, 2.95 mmol). The mixture stirred for 4 h at room temperature and was diluted with dichloromethane. The diluted mixture was washed with 1 N HCl (1×). The organics were washed with brine, dried (Na₂SO₄) and concentrated to give the desired product (0.80 g, 91%). ¹H NMR 300 MHz (CDCl₃) δ 7.70 (d, 2H), 7.29 (d, 2H), 4.65-4.55 (m, 1H), 3.64 (s, 2H), 2.91 (d, 2H), 2.39 (s, 3H), 1.34 (s, 9H), 0.80 (s, 6H).

### Step 3: {3-[8-Chloro-3-methyl-4-oxo-2-(tetrahydro-pyran-2-yl)-2,4-dihydro-pyrazolo[4,3-c]quinolin-5-yl]-2,2-dimethyl-propyl}-carbamic acid tert-butyl ester

To a solution of 8-Chloro-3-methyl-2-(tetrahydro-pyran-2-yl)-2,5-dihydro-pyrazolo[4,3-c]quinolin-4-one (0.14 g, 0.15 mmol), tosylate from Step 2 (0.40 g, 1.12 mmol), and potassium carbonate (0.31 g, 2.25 mmol) in toluene was added 18-crown-6 ether (0.59 g. 2.25 mmol). The reaction mixture was heated to reflux over night. The mixture was cooled to room temperature and diluted with water. The mixture was extracted with dichlormethane (3×), washed with brine, dried (Na₂SO₄), and concentrated. The residue was purified by chromatography on silica eluting with 0% to 30% ethyl acetate/hexane mixture to yield the desired product. LCMS: ES⁺ 503 (M+1).

### Step 4: 5-(3-Amino-2,2-dimethyl-propyl)-8-chloro-3-methyl-2,5-dihydro-pyrazolo[4,3-c]quinolin-4-one

Acidic deprotection as described in Example 202, Step 7 provided the title compound as a white solid. LCMS: Method FA, Rₜ=0.97 min, [MH⁺=319]. ¹H NMR 300 MHz (MeOH) δ 8.37-8.34 (1H, m), 7.97 (1H, s), 7.57-7.54 (1H, m), 4.62 (2H, t), 3.33 (2H, s), 3.23-3.16 (4 H, m), 3.04-3.03 (3H, m), 2.85-2.84 (3H, m), 2.35-2.25 (2H, m).

### EXAMPLE 212

### Preparation of 8-Chloro-3-methyl-5-(2-piperidin-2-yl-ethyl)-2,5-dihydro-pyrazolo[4,3-c]quinolin-4-one

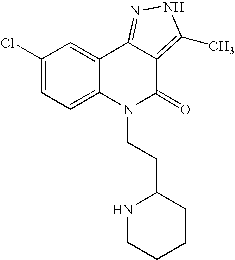

The title compound was prepared from the appropriate reagents by an analogous procedure to Example 202.

The necessary alkyation agent, 2-(2-Bromo-ethyl)-piperidine-1-carboxylic acid tert-butyl ester, was prepared via bromination according to the following procedure:

### Step 1: Preparation of 2-(2-Hydroxy-ethyl)-piperidine-1-carboxylic acid tert-butyl ester

To a solution of 2-piperidin-2-yl-ethanol (8.0 g, 61.9 mmol) in CH₂Cl₂ (200 mL) was added Boc₂O (13.5 g, 61.9 mmol) at 22° C., and the mixture was stirred for 4 h. The reaction mixture was then washed with 100 mL of a 0.2 M aqueous HCl solution, followed by 200 mL of H₂O, and finally 200 mL of brine. The organic layer was dried over MgSO₄, filtered and concentrated to provide 13.6 g (59.2 mmol) of the title compound in 96% yield. ¹H NMR 300 MHz (CDCl₃) δ 4.36-4.50 (m, 1H), 3.87-4.03 (m, 1H), 3.51-3.66 (m, 1H), 3.27-3.45 (m, 1H), 2.58-2.74 (m, 1H), 1.87-2.01 (m, 1H), 1.66-1.80 (m, 1H), 1.47 (s, 9H), 1.28-1.65 (m, 6H).

### Step 2: Preparation of 2-(2-Bromo-ethyl)-piperidine-1-carboxylic acid tert-butyl ester

To a solution of 2-(2-hydroxy-ethyl)-piperidine-1-carboxylic acid tert-butyl ester (14.4 g, 62.8 mmol) in CH₂Cl₂ (200 mL) was added PPh₃ (18.1 g, 69.0 mmol) followed by a solution of CBr₄ (22.9 g, 69.0 mmol) in 60 mL of CH₂Cl₂ at 22° C., and the mixture was stirred for 45 min. The reaction mixture was condensed in vacuo and then immediately purified through silica gel chromatography eluting the product with a gradient of 0 to 15% EtOAc in hexanes to providel 3.9 g (42.5 mmol) of the title compound in 68% yield. ¹H NMR 300 MHz (CDCl₃) δ 4.33-4.44 (m, 1H), 3.94-4.08 (m, 1H), 3.25-3.42 (m, 2H), 2.65-2.80 (m, 1H), 2.24-2.41 (m, 1H), 1.81-1.99 (m, 1H), 1.49-1.70 (m, 6H), 1.46 (s, 9H).

### Step 3: Preparation of 8-Chloro-3-methyl-5-(2-piperidin-2-yl-ethyl)-2,5-dihydro-pyrazolo[4,3-c]quinolin-4-one

To a solution of 8-chloro-3-methyl-2,5-dihydro-pyrazolo[4,3-c]quinolin-4-one (0.4 g, 1.4 mmol) in DMF (15 mL) was added bromide from Step 2 (1.0 g, 3.4 mmol) followed by NaH (60% dispersion, 0.1 g, 2.7 mmol) at 22° C., and the mixture was stirred for 24 h. The reaction mixture was treated with 20 ML H₂O, and the product extracted with 50 mL EtOAc. The organic layer was washed 3× with H₂O, dried over MgSO₄, filtered and concentrated to give crude product as a white solid. Silica gel chromatography eluting the product with a gradient of 0 to 50% EtOAc in hexanes afforded 0.2 g (0.4 mmol) of 2-{2-[8-chloro-3-methyl-4-oxo-2-(tetrahydro-pyran-2-yl)-2,4-dihydro-pyrazolo[4,3-c]quinolin-5-yl]-ethyl}-piperidine-1-carboxylic acid tert-butyl ester in 27% yield. LCMS: Method AA, Rₜ=2.63 min, [MH⁺=529.3].

### Step 4: Preparation of 8-Chloro-3-methyl-5-(2-piperidin-2-yl-ethyl)-2,5-dihydro-pyrazolo[4,3-c]quinolin-4-one

The HCl salt of the title compound was prepared after deprotection as described in Example 202, Step 7. ¹H NMR 300 MHz (DMSO) δ 8.80-8.98 (bm, 2H), 8.20 (s, 1H), 7.52-7.69 (m, 2H), 7.52-7.69 (m, 2H), 4.24-4.40 (m, 2H), 3.18-3.31 (m, 1H), 3.02-3.17 (m, 1H), 2.73-2.91 (m, 1H), 2.57 (s, 3H), 1.32-2.08 (m, 8H). LCMS: Method FA, Rₜ=1.13 min, [MH⁺=345.1].

### EXAMPLE 213 AND 214

### Preparation of 8-Chloro-3-methyl-5-(2R-piperidin-2-yl-ethyl)-2,5-dihydro-pyrazolo[4,3-c]quinolin-4-one and 8-Chloro-3-methyl-5-(2S-piperidin-2-yl-ethyl)-2,5-dihydro-pyrazolo[4,3-c]quinolin-4-one

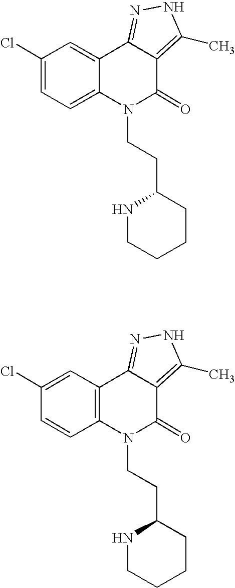

The title compounds were isolated by chiral HPLC separation of an intermediate from

Example 212. The intermediate from Step 3 was selectively deprotected to give the Boc protected racemate.

To a solution of 2-{2-[8-chloro-3-methyl-4-oxo-2-(tetrahydro-pyran-2-yl)-2,4-dihydro-pyrazolo[4,3-c]quinolin-5-yl]-ethyl}-piperidine-1-carboxylic acid tert-butyl ester (0.19 g, 0.37 mmol) in 3 mL MeOH was added 0.74 mL of a 1M HCl solution in Et₂O at 22° C., and the mixture was stirred for 15 min. The reaction mixture was then treated with Et₃N (0.26 uL, 1.85 mmol) and the solvent removed in vacuo. The resulting white solid was purified through a pad of silica gel using 50% EtOAc in hexanes to provide 0.15 g (0.33 mmol) of 2-[2-(8-chloro-3-methyl-4-oxo-2,4-dihydro-pyrazolo[4,3-c]quinolin-5-yl)-ethyl]-piperidine-1-carboxylic acid tert-butyl ester for 92% recovery. LCMS: Method FA, Rₜ=2.35 min, [MH⁺=445.3].

The pure racemic mixture was separated into enantiomers by prep HPLC chiral chromatography using a CHIRALCEL OD column: 10/90 EtOH/Hexane. Enantiomers had retention times of 1.4 (peak 1) and 2.2 (peak 2) minutes.

The HCl salt of each enantiomer was prepared after deprotection as described in Example 202, Step 7. Peak 1 LCMS: Method FA, Rₜ=1.05 min, [MH⁺=345.2]. Peak 2 LCMS: Method FA, Rₜ=1.09 min, [MH⁺=345.2].

### EXAMPLE 215

### Preparation of 8-Chloro-3-(2-methoxy-ethyl)-5-(2-piperidin-2-yl-ethyl)-2,5-dihydro-pyrazolo[4,3-c]quinolin-4-one

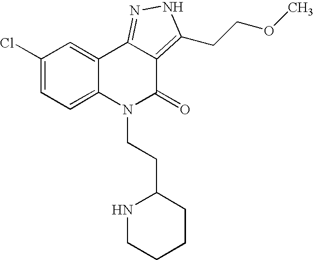

The title compound was prepared by methods outlined in Example 212 using appropriate reagents. ¹H NMR 300 MHz (DMSO) δ 8.68-8.85 (bm, 2H), 8.20 (s, 1H), 7.53-7.69 (m, 2H), 4.28-4.38 (m, 2H), 3.68 (t, 2H), 3.22 (s, 3H), 3.01-3.13 (m, 1H), 2.73-2.86 (m, 1H), 2.57 (s, 3H), 1.28-2.06 (m, 1OH). LCMS: Method FA, Rₜ=0.99 min, [MH⁺=389.2].

### EXAMPLE 216

### Preparation of 5-(3-Amino-butyl)-8-chloro 3-methyl-2,5-dihydro-pyrazolo[4,3-c]quinolin-4-one

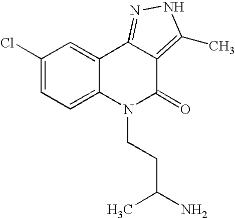

The title compound was prepared from the appropriate reagents by an analogous procedure to Example 212.

The necessary alkyation agent was prepared via bromination according to the following procedure.

### Step 1: Preparation of 3-tert-Butoxycarbonylamino-butyric acid ethyl ester

The title compound was obtained from Boc protection of ethyl 3-aminobutyrate according to methods outlined in Example 212, Step 1 (90% yield). ¹H NMR 300 MHz (CDCl₃) δ 4.85-5.00 (m, 1H), 4.14 (q, 2H), 3.93-4.08 (m, 1H), 2.48 (t, 2H), 1.43 (s, 9H), 1.26 (t, 3H), 1.20 (d, 3H).

### Step 2: Preparation of (3-Hydroxy-1-methyl-propyl)-carbamic acid tert-butyl ester

To a solution of 3-tert-butoxycarbonylamino-butyric acid ethyl ester (3.0 g, 12.8 mmol) in dry THF (26 mL) cooled to −15° C. was added dropwise a 1M solution of BH₃ in THF (16.6 mL, 16.6 mmol), and the mixture was allowed to warm to 22° C. and stirred for 12 h. The reaction mixture was then cooled to 0° C. and treated with 50 mL H₂O followed by 8.0 g K₂CO₃. The product was extracted from the aqueous layer with 3×100 mL EtOAc, and the combined organic layers dried over MgSO₄, filtered and concentrated in vacuo to provide a clear oil. The oil was purified through silica gel chromatography, eluting the product with a gradient of 0 to 50% EtOAc in hexanes to provide1.2 g (6.2 mmol) of the title compound in 48% yield. ¹H NMR 300 MHz (CDCl₃) δ 3.78-3.92 (m, 1H), 3.57-3.61 (m, 2H), 1.70-1.84 (m, 1H), 1.41 (s, 9H), 1.25-1.38 (m, 1H), 1.15 (d, 3H).

### Step 3: Preparation of (3-Bromo-1-methyl-propyl)-carbamic acid tert-butyl ester

Bromination according to Example 212, Step 2 using (3-hydroxy-1-methyl-propyl)-carbamic acid tert-butyl ester afforded the bromide (85% yield). ¹H NMR 300 MHz (CDCl₃) δ 4.22-4.42 (m, 1H), 3.68-3.86 (m, 1H), 3.31-3.43 (m, 2H), 1.87-2.04 (m, 2H), 1.42 (s, 9H), 1.14 (d, 3H).

### Step 4: Preparation of 5-(3-Amino-butyl)-8-chloro-3-methyl-2,5-dihydro-pyrazolo[4,3-c]quinolin-4-one

Alkylation according to Example 202, step 6 afforded the alkylated product in 44% yield. LCMS: Method FA, Rₜ=2.40 min, [MH⁺=489.8].

The HCl salt of the title compound was prepared after deprotection as described in Example 202, Step 7. ¹H NMR 300 MHz (DMSO) δ 8.19 (s, 1H), 7.86-8.05 (bm, 2H), 7.55-7.68 (m, 2H), 4.25-4.38 (m, 2H), 3.19-3.32 (m, 1H), 2.57 (s, 3H), 1.86-2.02 (m, 1H), 1.70-1.85 (m, 1H), 1.28 (d, 3H). LCMS: Method FA, Rₜ0.92 min, [MH⁺=305.2].

### EXAMPLE 217 AND 218

### Preparation of 5-(3R-Amino-butyl)-8-chloro 3-methyl-2,5-dihydro-pyrazolo[4,3-c]quinolin-4-one and of 5-(3S-Amino-butyl)-8-chloro 3-methyl-2,5-dihydro-pyrazolo[4,3-c]quinolin-4-one

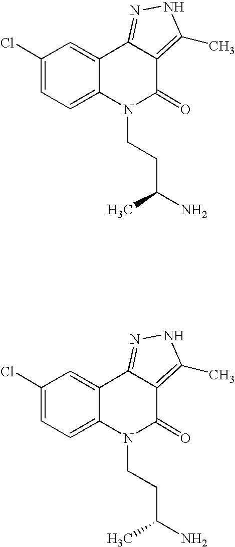

The title compounds were isolated by chiral HPLC separation of an intermediate from

Example 216. The intermediate from Step 3 was selectively deprotected to give the Boc protected racemate as described in Example 213 and 214 (LCMS: Method FA, Rₜ=1.78 min, [MH⁺=405.2]).

The pure racemic mixture was separated into enantiomers by prep HPLC chiral chromatography using a CHIRALPAK AD column: 5/95 Isopropanol/Hexane with 0.1% diethylamine. Enantiomers had retention times of 4.0 (peak 1) and 6.1 (peak 2) minutes.

The HCl salt of each enantiomer was prepared after deprotection as described in Example 202, Step 7. Peak 1 LCMS: Method AA, Rₜ=1.08 min, [MH⁺=305.1]. Peak 2 LCMS: Method AA, Rₜ=1.10 min, [MH⁺=305.1].

### EXAMPLE 219

### Preparation of 5-(3-Amino-butyl)-8-chloro 3-(2-methoxy-ethyl-2,5-dihydro-pyrazolo[4,3-c]quinolin-4-one

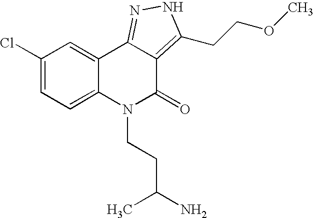

The title compound was prepared my methods analogous to those described in Example 216. (Alkylation product LCMS: Method FA, Rₜ=2.94 min, [MH⁺=533.3]).

The HCl salt of the title compound was prepared as described Example 212. ¹H NMR 300 MHz (DMSO) δ 8.24 (s, 1H), 7.90-8.12 (bm, 2H), 7.53-7.73 (m, 2H), 4.27-4.40 (m, 2H), 3.71 (t, 2H), 3.57 (s, 3H), 3.37-3.54 (m, 1H), 1.88-2.03 (m, 1H), 1.69-1.87 (m, 1H), 1.30 (d, 3H). LCMS: Method FA, Rₜ=0.99 min, [MH⁺=349.6].

### EXAMPLE 220

### Preparation of 5-(3-Amino-2S-methyl-propyl)-8-chloro-3-methyl-2,5-dihydro-pyrazolo[4,3-c]quinolin-4-one

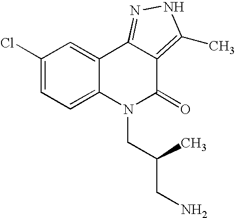

Prepared from the appropriate reagents by an analogous procedure to Example 212.

LC/MS: FA, Rₜ=0.91, E⁺=305.17. ¹H NMR (300 MHz, CDCl₃) δ 7.91 (br s, 1 H), 7.80 (d, 1 H), 7.07-6.95 (m, 2 H), 4.11-3.96 (m, 1 H), 3.70-3.56 (m, 1 H), 2.54-2.30 (m, 1 H), 2.23 (s, 3 H), 2.21-1.97 (m, 4 H), 0.74 (d, 3 H).

### EXAMPLE 221

### Preparation of 5-(3-Amino-2R-methyl-propyl)-8-chloro-3-methyl-2,5-dihydro-pyrazolo[4,3-c]quinolin-4-one

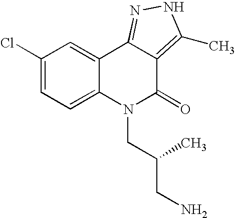

Prepared from the appropriate reagents by an analogous procedure to Example 212.

¹H NMR 300 MHz (DMSO) δ 8.35 (1H, s), 8.13-8.12 (1H, m), 7.61-7.52 (2H, m) 4.29-4.20 (1H, m), 4.12-4.02 (1H, m), 2.66-2.56 (3H, m), 2.54 (3H, s) 2.16-2.06 (2H, m), 0.90 (3H, d). LCMS: ES⁺305 (M+1).

### EXAMPLE 222

### Preparation of 5-(3-Amino-2S-methyl-propyl)-8-chloro-3-(2-methoxy-ethyl)-2,5-dihydro-pyrazolo[4,3-c]quinolin-4-one

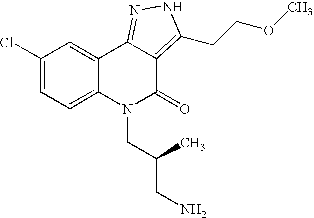

Prepared from the appropriate reagents by an analogous procedure to Example 212.

LCMS method FA, Rₜ=0.98, ES⁺349.59 (M+1). ¹H NMR (300 MHz, MeOD) δ 8.17 (1 H, d), 7.61-7.57 (2 H, m), 4.55 (1 H, dd), 4.10 (1 H, dd), 3.80 (2 H, t), 3.36 (2 H, t), 3.35 (3 H, s), 2.99-2.81 (2 H, m), 2.44 (1 H, br s), 1.20 (3 H, d).

### EXAMPLE 223

### Preparation of 5-(3-Amino-2R-methyl-propyl)-8-chloro-3-(2-methoxy-ethyl)-2,5-dihydro-pyrazolo[4,3-c]quinolin-4-one

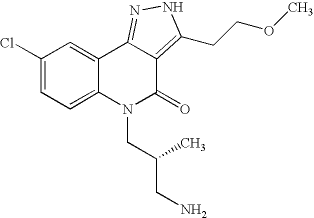

Prepared from the appropriate reagents by an analogous procedure to Example 212. ¹H NMR 300 MHz (DMSO) δ 8.30-7.97 (2H, 7.71-7.53 (2H, m), 4.39-4.24 (1H, m), 4.19-4.07 (1H, m), 3.70 (2H, t), 3.28 (3H, s), 2.81-2.64 (2H, m), 2.35-2.24 (1H, m), 1.37-1.19 (2H, m), 1.03-0.95 (3H, m), 0.89-0.80 (1H, m). LCMS method FA, Rₜ0.98, ES⁺349.59 (M+1).

### EXAMPLE 224

### Preparation of 8-Chloro-3-methyl-5R-piperidin-3-ylmethyl-2,5-dihydro-pyrazolo[4,3-c]quinolin-4-one

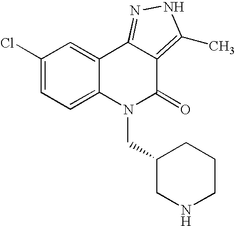

Prepared from the appropriate reagents by an analogous procedure to Example 212.

¹H NMR 300 MHz (MeOH) δ 8.08-8.07 (1H, m), 7.56-7.55 (2H, m), 4.52-4.44 (1H, m), 4.17-4.09 (1H, m), 3.27-3.21 (1H, m), 2.66 (3H, s), 2.37-2.30 (1H, m), 2.02-1.92 (2H, m), 1.75-1.51 (2H, m). LCMS: ES⁺331(M+1).

### EXAMPLE 225

### Preparation of of 8-Chloro-3-methyl-5S-piperidin-3-ylmethyl-2,5-dihydro-pyrazolo[4,3-c]quinolin-4-one

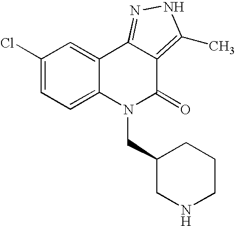

Prepared from the appropriate reagents by an analogous procedure to Example 212.

LC/MS: FA, Rₜ=0.91, ES⁺331.09 (M+1). ¹H NMR (300 MHz, CD₃OH) δ 8.28 (s, 1 H), 7.70 (s, 2 H), 4.73-4.62 (m, 1 H), 4.36-4.22 (m, 1 H), 3.42-3.33 (m, 4 H), 3.20-3.02 (m, 2 H), 2.81 (s, 3 H), 2.60-2.40 (m, 1 H), 2.20-2.0 (m, 2 H), 1.97-1.62 (m, 2 H).

### EXAMPLE 226

### Preparation of 8-Chloro-3-(2-methoxy-ethyl)-5R-piperidin-3-ylmethyl-2,5-dihydro-pyrazolo[4,3-c]quinolin-4-one

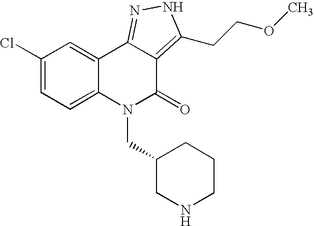

Prepared from the appropriate reagents by an analogous procedure to Example 212 and 205. ¹H NMR 300 MHz (DMSO) δ 9.03-8.95 (1H, m), 8.62-8.47 (1H, m), 8.24 (1H, s), 7.68-7.56 (2H, m), 4.39-4.28 (1H, m), 4.15-4.06 (1H, m), 3.75 (2H, t), 3.31-2.66 (2H, m) 3.23 (3H, s), 2.82-2.69 (2H, m), 2.31-2.16 (1H, m), 1.85-1.69 (3H, m), 1.66-1.48 (1H, m), 1.43-1.22 (2H, m). LCMS: ES⁺375 (M+1).

### EXAMPLE 227

### Preparation of 8-Chloro-3-(2-methoxy-ethyl)-5S-piperidin-3-ylmethyl-2,5-dihydro-pyrazolo[4,3-c]quinolin-4-one

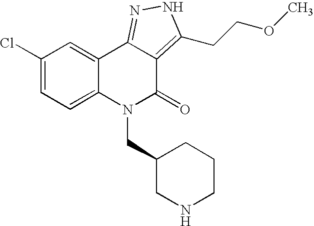

Prepared from the appropriate reagents by an analogous procedure to Example 212 and 205. ¹H NMR 300 MHz (MeOH) δ 8.20-8.19 (1H, m), 7.62-7.60 (2H, m), 4.64-4.54 (1H, m), 4.23-4.15 (1H, m), 3.83 (2H, t), 3.81 (3H, s), 3.05-3.24 (2H, m), 3.08-2.93 (3H, m), 2.41-2.34 (1H, m), 2.09-1.99 (3H, m), 1.83-1.60 (2H, m). LCMS: ES⁺375 (M+1).

### EXAMPLE 228

### Preparation of 5-Azetidin-3-ylmethyl-8-chloro-3-methyl-2,5-dihydro-pyrazolo[4,3-c]quinolin-4-one

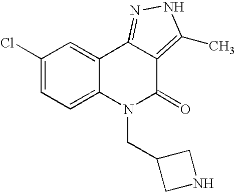

The title compound was prepared from the appropriate reagents by an analogous procedure to Example 212.

The necessary alkyation agent was prepared from azetidine-1,3-dicarboxylic acid mono tert-butyl ester.

### Step 1: Preparation of 3-Hydroxymethyl-azetidine-1-carboxylic acid tert-butyl ester

A solution of azetidine-1,3-dicarboxylic acid mono-tert-butyl ester (0.48 g, 2.38 mmol) and N-methylmorpholine (0.24 g, 2.38 mmol) in THF was cooled to −10° C. Isobutyl chloroformate (0.33 g, 2.38 mmol) was added dropwise. The mixture stirred for 15 min., and the liquid portion was added to a mixture of NaBH₄ (0.18 g, 4.77 mmol) in 50 ml of ice water. The combined mixture stirred for 30 minutes and was poured into and aqueous solution of sodium bicarbonate and extracted 2× with dichloromethane. The organic fractions were combined, washed with brine, dried (Na₂SO₄), and concentrated to yield the desired product.

### Step 2: Preparation of 3-Bromomethyl-azetidine-1-carboxylic acid tert-butyl ester

Bromination of 3-Hydroxymethyl-azetidine-1-carboxylic acid tert-butyl ester as described in Example 212, Step 2 provided the necessary alkylating agent.

### Step 3: Preparation of 3-[8-Chloro-3-methyl-4-oxo-2-(tetrahydro-pyran-2-yl)-2,4-dihydro-pyrazolo[4,3-c]quinolin-5-ylmethyl]-azetidine-1-carboxylic acid tert-butyl ester

Alkylation according to the method outlined in Example 202, Step 6 affords the alkyated product.

### Step 4: Preparation of 5-Azetidin-3-ylmethyl-8-chloro-3-methyl-2,5-dihydro-pyrazolo[4,3-c]quinolin-4-one

Acidic deprotection as described in Example 202, Step 7 provided the title compound as a white solid. LCMS: Method FA, Rₜ=0.93 min, [MH⁺=303].

### EXAMPLE 229

### Preparation of 5-(3-Amino-3-methyl-butyl)-8-chloro-3-methyl-2,5-dihydro-pyrazolo[4,3-c]quinolin-4-one

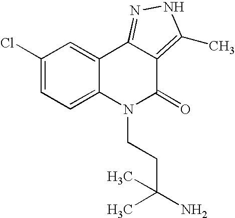

### Step 1: Preparation of Sulfamic acid 3-methyl-butyl ester

Chlorosulfonyl isocyanate (12.5 mL, 143 mmol) was stirred at 0° C. To this solution was added formic acid (543 mL, 143 mmol) dropwise over 30 minutes. Methylene chloride (10 mL) was added to facilitate stirring, and this was allowed to warm to RT overnight. Methylene chloride (75 mL) was added, and the reaction was cooled to 0° C. A mixture of pyridine (21.0 mL, 259 mmol) and 3-methyl-butan-1-ol (18.0 mL, 173 mmol) in methylene chloride (75 mL) was added dropwise over 1 h. This was allowed to warm to rt and stirred until TLC indicated complete disappearance of starting alcohol (95:5 CH₂Cl₂/EtOAc eluant, R_{f}=0.7) and appearance of the desired product (95:5 CH₂Cl₂/EtOAc eluant, R_{f}=0.8). The reaction mixture was added to saturated NH₄Cl (100 mL) and the reaction was washed (4×100 mL). The aqueous layers were extracted once with chloroform, and the combined organic fractions were concentrated in vacuo.

The crude reaction mixture was then be purified by flash chromatography (110 g prepacked Isco™ cartridge, hexanes to methylene chloride gradient), to give a colourless oil (10.95 g, 48%). ¹HNMR (300 MHz, CDCl₃) δ 4.15 (2H, dd), 1.69 (1H, dddd), 1.55 (2H, dd), 0.56 (6H, d). LCMS: Method FA Rₜ=1.47 min, EI⁻=166.1.

### Step 2: Preparation of 4,4-Dimethyl-[1,2,3]oxathiazinane 2,2-dioxide

To a solution of sulfamic acid from Step 1 (8.201 g, 49.4 mmol) in methylene chloride (500 mL) was added iodobenzene diacetate (17.5 g, 54.3 mmol) then a catalytic amount of rhodium acetate dimer, follwed by manganese oxide (6.2 g, 114 mmol). This was stirred at RT for 2 h, until TLC indicated disappearance of starting material and appearance of the desired product (Et₂O eluant, R_{f}=0.3). The solids were removed by filtration through sodium sulfate and the resulting green solution was concentrated in vacuo. The green oil was dissolved in diethyl ether and filtered through a pad of silica. Hexanes were added and the mixture was stirred for 1 h. The resulting tan solid was collected by filtration to give the title compound (7.95 g, 97%). ¹HNMR (300 MHz, CD₃Cl) δ 4.68 (2H, dd), 1.78 (2H, dd), 1.43 (6H, s).

### Step 3: Preparation of 3-Allyl-4,4-dimethyl-[1,2,3]oxathiazinane 2,2-dioxide

The sulfonamide from Step 2 (7.95 g, 48.2 mmol) was stirred in methylene chloride (350 mL) at RT. To this reaction mixture was added benzyl tributyl ammonium bromide (858 mg, 2.41 mmol), allyl bromide (16.7 mL, 192.8 mmol) followed by 5.0 N NaOH (70 mL). This was allowed to stir at RT for 2 h, at which point TLC indicated disappearance of starting material (Et₂O eluant, R_{f}=0.3) and appearance of desired product (R_{f}=0.7). The organic layer was separated, and the aqueous layer was extracted with chloroform (3×50 mL) and dried, and the combined organic layers were concentrated in vacuo. The crude product was dissolved in 9:1 hexanes/Et₂O and filtered through a plug of silica, followed by 4:1 hexanes/Et₂O, then 3:2 hexanes/Et₂O. The fractions containing the product were concentrated in vacuo to give the desired product as a pale yellow oil (8.2 g, 83%). ¹HNMR (300 MHz, CD₃Cl) δ 5.86-5.99 (1H, m), 5.26 (1H, dd), 5.14 (1H, dd), 3.83 (2H, dd), 1.95 (2H, dd), 1.40 (6H, s).

### Step 4: Preparation of 5-(3-Allylamino-3-methyl-butyl)-8-chloro-3-methyl-2-(tetrahydro-pyran-2-yl)-2,5-dihydro-pyrazolo[4,3-c]quinolin-4-one

To a solution of 8-Chloro-3-methyl-2-(tetrahydro-pyran-2-yl)-2,5-dihydro-pyrazolo[4,3-c]quinolin-4-one (200 mg, 0.629 mmol) in DMSO (6 mL) was added sodium hydride (63 mg, 60% dispersion in oil, 1.57 mmol). This was allowed to stir at room temperature for about 30 minutes, at which time gas evolution had ceased and the mixture was mostly homogenous. A solution of the sulfonamide from Step 3 (320 mg, 1.57 mmol) in DMSO (0.5 mL) was added, and the reaction was stirred at RT for 5 d, at which point LCMS indicated disappearance of starting material and the presence of the desired product, in addition to the undesired O-alkyl derivative. The reaction mixture was diluted with water (50 mL) and solid NaHCO₃ was added until the reaction achieved pH 8. Solid sodium chloride was added until the solution was saturated, and the mixture was allowed to stir at rt for one hour. The resulting white precipitate was collected, rinsed with water, dried, and triturated in ethyl acetate to give a 1:1 mixture of the N- and O-alkyl products (167 mg, 60%). The isomers could also be separated by reverse phase HPLC to give the desired N-alkyl derivative as a white solid. ¹HNMR (300 MHz, CD₃OD) δ 8.86 (1H, d), 7.68 (1H, dd), 7.59 (1H, d), 6.09 (1H, dddd), 5.84 (1H, dd), 5.66 (1H, dd), 5.56 (1H, dd), 4.51 (2H, ddd), 4.18 (2H, dd), 3.94 (2H, m), 3.78 (2H, d), 2.95 (3H, s), 2.67 (2H, m), 1.81-2.34 (4 H, m), 1.61 (6H, s). LCMS: Method AA Rₜ=1.65 min, EI⁺=443.3.

### Step 5: Preparation of 5-(3-Amino-3-methyl-butyl)-8-chloro-3-methyl-2-(tetrahydro-pyran-2-yl)-2,5-dihydro-pyrazolo[4,3-c]quinolin-4-one

The allyl amine from Step 4 (518 mg, 1.17 mmol) was stirred with diethyl amine (0.484 mL, 4.68 mmol), sodium bicarbonate (196 mg, 2.34 mmol) and palladium tetrakis(triphenyl phosphine) (67 mg, 0.86 mmol) in acetonitrile at reflux for 6 h, at which point LCMS indicated disappearance of starting material and the appearance of the desired primary amine. The reaction was cooled to rt and water (50 mL) was added. The resulting tan solid was collected by filtration. Sodium chloride was added to the solution, which was then extracted with chloroform (5×25 mL). The combined organic fractions and the tan solid were combined, and the solvent removed in vacuo. The reaction was then purified by reverse phase HPLC to give the title compound as a white solid (27 mg, 6%). ¹HNMR (300 MHz, CD₃OD) δ 8.83 (1H, dd), 7.68 (1H, dd), 7.58 (1H, dd), 5.80 (1H, dd), 4.51 (2H, dd), 4.19 (2H, dd), 3.89-3.98 (2H, m), 2.94 (3H, s), 2.63-2.69 (2H, m), 1.81-2.83 (4 H, m), 1.58 (6H, s). LCMS: Method AA Rₜ=1.52 min, EI⁺=403.2.

### Step 6: Preparation of 5-(3-Amino-3-methyl-butyl)-8-chloro-3-methyl-2,5-dihydro-pyrazolo[4,3-c]quinolin-4-one

Acidic deprotection as in Example 202, Step 7 provided the title compound as a white solid. ¹HNMR (300 MHz, CD₃OD) δ 8.21 (1H, d), 7.73 (1H, dd), 7.64 (1H, d), 4.53 (2H, ddd), 2.81 (3H, s), 2.16 (2H, ddd), 1.64 (6H, s). LCMS: Method AA Rₜ=1.08 min, EI⁺=319.2.

### EXAMPLE 230

### Preparation of 8-Chloro-3-methyl-5-(2-oxo-2-pyrrolidin-2-yl-ethyl)-2,5-dihydro-pyrazolo[4,3-c]quinolin-4-one

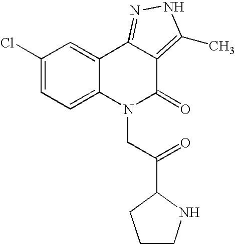

The title compound was prepared by methods outlined in Example 202 using Boc protected 2-Chloro-1-pyrrolidin-2-yl-ethanone in the alkylation step. ¹H NMR (300 MHz, DMSO-d₆) δ 9.67 (bs, 1H), 8.83 (bs, 1H), 8.24 (d, 1H), 7.54-7.64 (m, 2H), 5.30-5.57 (AB× multiplet, 2H), 4.82-4.92 (m, 1H), 3.18-3.32 (m, 2H), 2.60 (s, 3H), 2.48-2.58 (m, 2H), 2.19-2.31 (m, 1H), 1.87-2.08 (m, 2H).

### EXAMPLE 231

### Preparation of 8-Chloro-3-methyl-5-(3-methylamino-propyl)-2,5-dihydro-pyrazolo[4,3-c]quinolin-4-one

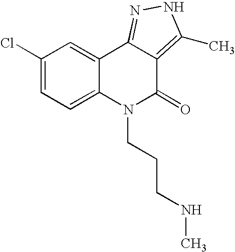

Title compound was prepared from {3-[8-chloro-3-methyl-4-oxo-2-(tetrahydro-pyran-2-yl)-2,4-dihydropyrazolo[4,3-c]quinolin-5-yl]-propyl}-carbamic acid tert-butyl ester obtained using the procedure outlined in Example 202.

### Step 1: Preparation of {3-[8-chloro-3-methyl-4-oxo-2-(tetrahydro-pyran-2-yl)-2,4-dihydropyrazolo[4,3-c]quinolin-5-yl]-propyl}-methyl-carbamic acid tert-butyl ester

To a solution of {3-[8-chloro-3-methyl-4-oxo-2-(tetrahydro-pyran-2-yl)-2,4-dihydropyrazolo[4,3-c]quinolin-5-yl]-propyl}-carbamic acid tert-butyl ester (0.23 g, 0.49 mmol) in anhyd. DMF (10 mL) was added NaH (60% dispersion, 0.028 g, 0.70 mmol) at 0° C., and the mixture was stirred for 30 min. MeI (0.035 mL, 0.56 mmol) was added and the solution was stirred for 1 h before water was added. The mixture was extracted with CH₂Cl₂. The organic layer was separated, dried over MgSO₄ and concentrated. Purification by C-18 RP LC-MS chromatography afforded {3-[8-chloro-3-methyl-4-oxo-2-(tetrahydro-pyran-2-yl)-2,4-dihydropyrazolo[4,3-c]quinolin-5-yl]-propyl}-methyl-carbamic acid tert-butyl ester (0.064 g, 27%). Purification by ISCO chromatography or recrystallization were also performed depending on the scale of the reaction. LCMS: Method FA, Rₜ=1.84 min, [MH⁺=489.2].

### Step 2: Preparation of 8-Chloro-3-methyl-5-(3-methylamino-propyl)-2,5-dihydro-pyrazolo[4,3-c]quinolin-4-one

To a solution of {3-[8-chloro-3-methyl-4-oxo-2-(tetrahydro-pyran-2-yl)-2,4 dihydropyrazolo[4,3-c]quinolin-5-yl]-propyl}-methyl-carbamic acid tert-butyl ester (0.064 g, 0.13 mmol) in 1 mL of MeOH was added 2.5 mL of HCl (4M in dioxane). The reaction was stirred for 12 h at 25° C. The reaction mixture was concentrated to afforded the title compound as a white solid (0.045 g, 100%). LCMS: Method FA, Rₜ=0.90 min, [MH⁺=305.2]. ¹H NMR 300 MHz (CD₃OD) δ 8.12-8.14 (dd, 1H), 7.57-7.59 (m, 2H), 4.40-4.46 (t, 2H), 3.00-3.07 (t, 2H), 2.71 (s, 3H), 2.66 (s, 3H), 2.07-2.19 (m, 2H).

### EXAMPLE 232

### Preparation of 5-(3-Amino-propyl)-8-fluoro-3-(2-methoxy-ethyl)-2,5-dihydro-pyrazolo[4,3-c]quinolin-4-one

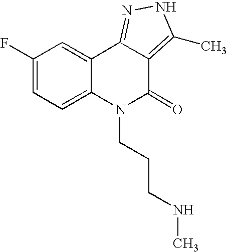

The title compound was prepared in an analogous manner to Example 231.

{3-[8-Fluoro-3-methyl-4-oxo-2-(tetrahydro-pyran-2-yl)-2,4-dihydro-pyrazolo[4,3-c]quinolin-5-yl]-propyl}-methyl-carbamic acid tert-butyl ester: To a solution of {3-[8-Fluoro-3-methyl-4-oxo-2-(tetrahydro-pyran-2-yl)-2,4-dihydro-pyrazolo[4,3-c]quinolin-5-yl]-propyl}-carbamic acid tert-butyl ester (158 mg, 0.345 mmol) in DMF was added NaH (36.0 mg, 0.900 mmol) and the mixture was stirred for 10 min before CH₃I (43.0 uL, 0.689 mmol) was added. After stirring for 18 h, the mixture was partitioned between H₂O and ethyl acetate, the aqueous layer was extracted with ethyl acetate (2×) and the combined organic layers were washed with H₂O (2×) and saturated NaCl solution before being dried over MgSO₄, filtered and concentrated in vacuo. Reverse phase chromatography on a C18 column eluting with a gradient of 60 to 100% CH₃CN in H₂O with 0.1% ammonium acetate afforded 96.0 mg (59%) of the title compound as a white solid. ¹H NMR (300 MHz, DMSO-d₆) δ 7.79 (dd, J=1.8, 8.5 Hz, 1H), 7.55 (dd, J=4.0, 9.5 Hz, 1H), 7.41 (ddd, J=1.5, 8.0, 8.0 Hz, 1H), 5.68 (d, J=8.5 Hz, 1H), 4.21 (br s, 2H), 3.89-3.98 (m, 1H), 3.66-3.78 (m, 1H), 3.18-3.29 (m, 1H), 2.75 (s, 3H), 2.32-2.46 (m, 1H), 1.90-2.11 (m, 3H), 1.70-1.89 (m, 3H), 1.54-1.65 (m, 3H), 1.33-1.48 (m, 2H), 1.16-1.31 (s, 9H); ¹⁹F NMR (282 MHz, DMSO-d₆) −121.58 to −121.47; LC/MS: AA standard Rₜ=2.26 min, EI⁺ 473.29.

### EXAMPLE 223

### Preparation of 3-Methyl-5-(3-methylamino-propyl)-2,5-dihydro-pyrazolo[4,3-c]quinolin-4-one

1H), 1.70-1.85 (m, 1H), 1.28 (d, 3H). LCMS: Method FA, Rₜ=0.92 min, [MH⁺=305.2].

### EXAMPLE 217 AND 218

### Preparation of 5-(3R-Amino-butyl)-8-chloro 3-methyl-2,5-dihydro-pyrazolo[4,3-c]quinolin-4-one and of 5-(3S-Amino-butyl)-8-chloro 3-methyl-2,5-dihydro-pyrazolo[4,3-c]quinolin-4-one

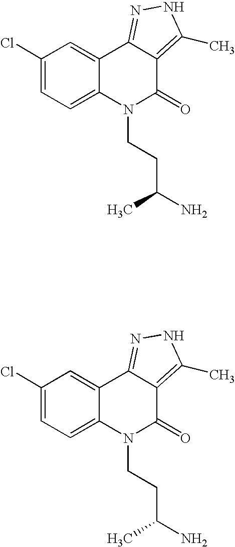

The title compounds were isolated by chiral HPLC separation of an intermediate from Example 216. The intermediate from Step 3 was selectively deprotected to give the Boc protected racemate as described in Example 213 and 214 (LCMS: Method FA, Rₜ=1.78 min, [MH⁺=405.2]).

The pure racemic mixture was separated into enantiomers by prep HPLC chiral chromatography using a CHIRALPAK AD column: 5/95 Isopropanol/Hexane with 0.1% diethylamine. Enantiomers had retention times of 4.0 (peak 1) and 6.1 (peak 2) minutes.

The HCl salt of each enantiomer was prepared after deprotection as described in Example 202, Step 7. Peak 1 LCMS: Method AA, Rₜ=1.08 min, [MH⁺=305.1]. Peak 2 LCMS: Method AA, Rₜ=1.10 min, [MH⁺=305.1 ].

### EXAMPLE 219

### Preparation of 5-(3-Amino-butyl)-8-chloro 3-(2-methoxy-ethyl-2,5-dihydro-pyrazolo[4,3-c]quinolin4-one

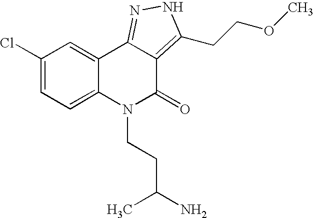

The title compound was prepared my methods analogous to those described in Example 216. (Alkylation product LCMS: Method FA, Rₜ=2.94 min, [MH⁺=533.3]).

The HCl salt of the title compound was prepared as described Example 212. ¹H NMR 300 MHz (DMSO) δ 8.24 (s, 1H), 7.90-8.12 (bm, 2H), 7.53-7.73 (m, 2H), 4.27-4.40 (m, 2H), 3.71 (t, 2H), 3.57 (s, 3H), 3.37-3.54 (m, 1H), 1.88-2.03 (m, 1H), 1.69-1.87 (m, 1H), 1.30 (d, 3H). LCMS: Method FA, Rₜ=0.99 min, [MH⁺=349.6].

### EXAMPLE 220

### Preparation of 5-(3-Amino-2S-methyl-propyl)-8-chloro-3-methyl-2,5-dihydro-pyrazolo[4,3-c]quinolin-4-one

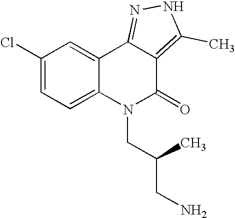

Prepared from the appropriate reagents by an analogous procedure to Example 212.

LC/MS: FA, Rₜ=0.91, E⁺=305.17. ¹H NMR (300 MHz, CDCl₃) δ 7.91 (br s, 1 H), 7.80 (d, 1 H), 7.07-6.95 (m, 2 H), 4.11-3.96 (m, 1 H), 3.70-3.56 (m, 1 H), 2.54-2.30 (m, 1 H), 2.23 (s, 3 H), 2.21-1.97 (m, 4 H), 0.74 (d, 3 H).

### EXAMPLE 221

### Preparation of 5-(3-Amino-2R-methyl-propyl)-8-chloro-3-methyl-2,5-dihydro-pyrazolo[4,3-c]quinolin-4-one

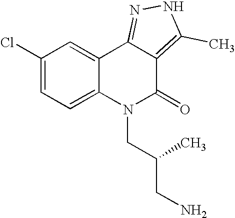

Prepared from the appropriate reagents by an analogous procedure to Example 212.

¹H NMR 300 MHz (DMSO) δ 8.35 (1H, s), 8.13-8.12 (1H, m), 7.61-7.52 (2H, m) 4.29-4.20 (1H, m), 4.12-4.02 (1H, m), 2.66-2.56 (3H, m), 2.54 (3H, s) 2.16-2.06 (2H, m), 0.90 (3H, d). LCMS: ES⁺305 (M+1).

### EXAMPLE 222

### Preparation of 5-(3-Amino-2S-methyl-propyl)-8-chloro-3-(2-methoxy-ethyl)-2,5-dihydro-pyrazolo[4,3-c]quinolin-4-one

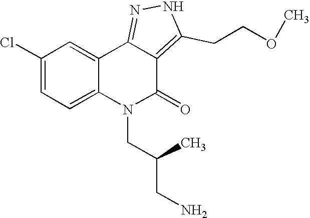

Prepared from the appropriate reagents by an analogous procedure to Example 212.

LCMS method FA, Rₜ=0.98, ES⁺349.59 (M+1). ¹H NMR (300 MHz, MeOD) δ 8.17 (1 H, d), 7.61-7.57 (2 H, m), 4.55 (1 H, dd), 4.10 (1 H, dd), 3.80 (2 H, t), 3.36 (2 H, t), 3.35 (3 H, s), 2.99-2.81 (2 H, m), 2.44 (1 H, br s), 1.20 (3 H, d).

### EXAMPLE 223

### Preparation of 5-(3-Amino-2R-methyl-propyl)-8-chloro-3-(2-methoxy-ethyl)-2,5-dihydro-pyrazolo[4,3-c]quinolin-4-one

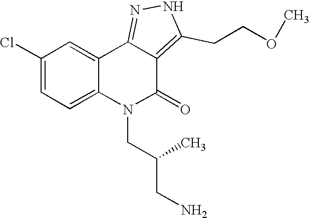

Prepared from the appropriate reagents by an analogous procedure to Example 212. ¹H NMR 300 MHz (DMSO) δ 8.30-7.97 (2H, 7.71-7.53 (2H, m), 4.39-4.24 (1H, m), 4.19-4.07 (1H, m), 3.70 (2H, t), 3.28 (3H, s), 2.81-2.64 (2H, m), 2.35-2.24 (1H, m), 1.37-1.19 (2H, m), 1.03-0.95 (3H, m), 0.89-0.80 (1H, m). LCMS method FA, Rₜ0.98, ES⁺349.59 (M+1).

### EXAMPLE 224

### Preparation of 8-Chloro-3-methyl-5R-piperidin-3-ylmethyl-2,5-dihydro-pyrazolo[4,3-c]quinolin-4-one

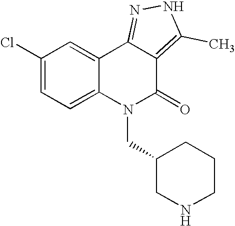

Prepared from the appropriate reagents by an analogous procedure to Example 212.

¹H NMR 300 MHz (MeOH) δ 8.08-8.07 (1H, m), 7.56-7.55 (2H, m), 4.52-4.44 (1H, m), 4.17-4.09 (1H, m), 3.27-3.21 (1H, m), 2.66 (3H, s), 2.37-2.30 (1H, m), 2.02-1.92 (2H, m), 1.75-1.51 (2H, m). LCMS: ES⁺331(M+1).

### EXAMPLE 225

### Preparation of of 8-Chloro-3-methyl-5S-piperidin-3-ylmethyl-2,5-dihydro-pyrazolo[4,3-c]quinolin-4-one

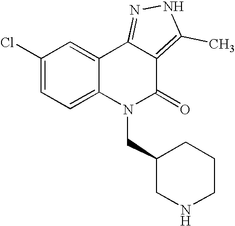

Prepared from the appropriate reagents by an analogous procedure to Example 212.

LC/MS: FA, Rₜ=0.91, ES⁺331.09 (M+1). ¹H NMR (300 MHz, CD₃OH) δ 8.28 (s, 1 H), 7.70 (s, 2 H), 4.73-4.62 (m, 1 H), 4.36-4.22 (m, 1 H), 3.42-3.33 (m, 4 H), 3.20-3.02 (m, 2 H), 2.81 (s, 3 H), 2.60-2.40 (m, 1 H), 2.20-2.0 (m, 2 H), 1.97-1.62 (m, 2 H).

### EXAMPLE 226

### Preparation of 8-Chloro-3-(2-methoxy-ethyl)-5R-piperidin-3-ylmethyl-2,5-dihydro-pyrazolo[4,3-c]quinolin-4-one

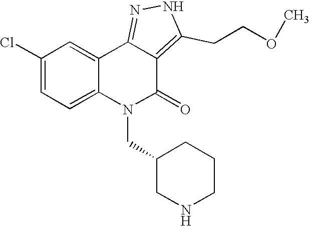

Prepared from the appropriate reagents by an analogous procedure to Example 212 and 205. ¹H NMR 300 MHz (DMSO) δ 9.03-8.95 (1H, m), 8.62-8.47 (1H, m), 8.24 (1H, s), 7.68-7.56 (2H, m), 4.39-4.28 (1H, m), 4.15-4.06 (1H, m), 3.75 (2H, t), 3.31-2.69 (2H, m) 3.23 (3H, s), 2.82-2.69 (2H, m), 2.31-2.16 (1H, m), 1.85-1.69 (3H, m), 1.66-1.48 (1H, m), 1.43-1.22 (2H, m). LCMS: ES⁺375 (M+1).

### EXAMPLE 227

### Preparation of 8-Chloro-3-(2-methoxy-ethyl)-5S-piperidin-3-ylmethyl-2,5-dihydro-pyrazolo[4,3-c]quinolin-4-one

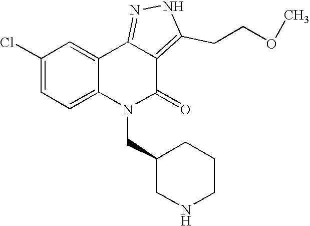

Prepared from the appropriate reagents by an analogous procedure to Example 212 and 205. ¹H NMR 300 MHz (MeOH) δ 8.20-8.19 (1H, m), 7.62-7.60 (2H, m), 4.64-4.54 (1H, m), 4.23-4.15 (1H, m), 3.83 (2H, t), 3.81 (3H, s), 3.05-3.24 (2H, m), 3.08-2.93 (3H, m), 2.41-2.34 (1H, m), 2.09-1.99 (3H, m), 1.83-1.60 (2H, m). LCMS: ES⁺375 (M+1).

### EXAMPLE 228

### Preparation of 5-Azetidin-3-ylmethyl-8-chloro-3-methyl-2,5-dihydro-pyrazolo[4,3-c]quinolin-4-one

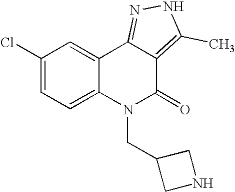

The title compound was prepared from the appropriate reagents by an analogous procedure to Example 212.

The necessary alkyation agent was prepared from azetidine-1,3-dicarboxylic acid mono tert-butyl ester.

### Step 1: Preparation of 3-Hydroxymethyl-azetidine-1-carboxylic acid tert-butyl ester

A solution of azetidine-1,3-dicarboxylic acid mono-tert-butyl ester (0.48 g, 2.38 mmol) and N-methylmorpholine (0.24 g, 2.38 mmol) in THF was cooled to −10° C. Isobutyl chloroformate (0.33 g, 2.38 mmol) was added dropwise. The mixture stirred for 15 min., and the liquid portion was added to a mixture of NaBH₄ (0.18 g, 4.77 mmol) in 50 ml of ice water. The combined mixture stirred for 30 minutes and was poured into and aqueous solution of sodium bicarbonate and extracted 2× with dichloromethane. The organic fractions were combined, washed with brine, dried (Na₂SO₄), and concentrated to yield the desired product.

### Step 2: Preparation of 3-Bromomethyl-azetidine-1-carboxylic acid tert-butyl ester

Bromination of 3-Hydroxymethyl-azetidine-1-carboxylic acid tert-butyl ester as described in Example 212, Step 2 provided the necessary alkylating agent.

### Step 3: Preparation of 3-[8-Chloro-3-methyl-4-oxo-2-(tetrahydro-pyran-2-yl)-2,4-dihydro-pyrazolo[4,3-c]quinolin-5-ylmethyl]-azetidine-1-carboxylic acid tert-butyl ester

Alkylation according to the method outlined in Example 202, Step 6 affords the alkyated product.

### Step 4: Preparation of 5-Azetidin-3-ylmethyl-8-chloro-3-methyl-2,5-dihydro-pyrazolo[4,3-c]quinolin-4-one

Acidic deprotection as described in Example 202, Step 7 provided the title compound as a white solid. LCMS: Method FA, Rₜ0.93 min, [MH⁺=303].

### EXAMPLE 229

### Preparation of 5-(3-Amino-3-methyl-butyl)-8-chloro-3-methyl-2,5-dihydro-pyrazolo[4,3-c]quinolin-4-one

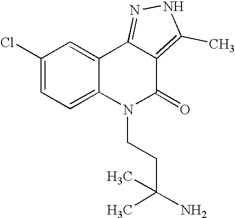

### Step 1: Preparation of Sulfamic acid 3-methyl-butyl ester

Chlorosulfonyl isocyanate (12.5 mL, 143 mmol) was stirred at 0° C. To this solution was added formic acid (543 mL, 143 mmol) dropwise over 30 minutes. Methylene chloride (10 mL) was added to facilitate stirring, and this was allowed to warm to RT overnight. Methylene chloride (75 mL) was added, and the reaction was cooled to 0° C. A mixture of pyridine (21.0 mL, 259 mmol) and 3-methyl-butan-1-ol (18.0 mL, 173 mmol) in methylene chloride (75 mL) was added dropwise over 1 h. This was allowed to warm to rt and stirred until TLC indicated complete disappearance of starting alcohol (95:5 CH₂Cl₂/EtOAc eluant, R_{f}=0.7) and appearance of the desired product (95:5 CH₂Cl₂/EtOAc eluant, R_{f}=0.8). The reaction mixture was added to saturated NH₄Cl (100 mL) and the reaction was washed (4×100 mL). The aqueous layers were extracted once with chloroform, and the combined organic fractions were concentrated in vacuo.

The crude reaction mixture was then be purified by flash chromatography (110 g prepacked Isco™ cartridge, hexanes to methylene chloride gradient), to give a colourless oil (10.95 g, 48%). ¹HNMR (300 MHz, CDCl₃) δ 4.15 (2H, dd), 1.69 (1H, dddd), 1.55 (2H, dd), 0.56 (6H, d). LCMS: Method FA Rₜ=1.47 min, EI⁻=166.1.

### Step 2: Preparation of 4,4-Dimethyl-[1,2,3]oxathiazinane 2,2-dioxide

To a solution of sulfamic acid from Step 1 (8.201 g, 49.4 mmol) in methylene chloride (500 mL) was added iodobenzene diacetate (17.5 g, 54.3 mmol) then a catalytic amount of rhodium acetate dimer, follwed by manganese oxide (6.2 g, 114 mmol). This was stirred at RT for 2 h, until TLC indicated disappearance of starting material and appearance of the desired product (Et₂O eluant, R_{f}=0.3). The solids were removed by filtration through sodium sulfate and the resulting green solution was concentrated in vacuo. The green oil was dissolved in diethyl ether and filtered through a pad of silica. Hexanes were added and the mixture was stirred for 1 h. The resulting tan solid was collected by filtration to give the title compound (7.95 g, 97%). ¹HNMR (300 MHz, CD₃Cl) δ 4.68 (2H, dd), 1.78 (2H, dd), 1.43 (6H, s).

### Step 3: Preparation of 3-Allyl-4,4-dimethyl-[1,2,3]oxathiazinane 2,2-dioxide

The sulfonamide from Step 2 (7.95 g, 48.2 mmol) was stirred in methylene chloride (350 mL) at RT. To this reaction mixture was added benzyl tributyl ammonium bromide (858 mg, 2.41 mmol), allyl bromide (16.7 mL, 192.8 mmol) followed by 5.0 N NaOH (70 mL). This was allowed to stir at RT for 2 h, at which point TLC indicated disappearance of starting material (Et₂O eluant, R_{f}=0.3) and appearance of desired product (R_{f}=0.7). The organic layer was separated, and the aqueous layer was extracted with chloroform (3×50 mL) and dried, and the combined organic layers were concentrated in vacuo. The crude product was dissolved in 9:1 hexanes/Et₂O and filtered through a plug of silica, followed by 4:1 hexanes/Et₂O, then 3:2 hexanes/Et₂O. The fractions containing the product were concentrated in vacuo to give the desired product as a pale yellow oil (8.2 g, 83%). ¹HNMR (300 MHz, CD₃Cl) δ 5.86-5.99 (1H, m), 5.26 (1H, dd), 5.14 (1H, dd), 3.83 (2H, dd), 1.95 (2H, dd), 1.40 (6H, s).

### Step 4: Preparation of 5-(3-Allylamino-3-methyl-butyl)-8-chloro-3-methyl-2-(tetrahydro-pyran-2-yl)-2,5-dihydro-pyrazolo[4,3-c]quinolin-4-one

To a solution of 8-Chloro-3-methyl-2-(tetrahydro-pyran-2-yl)-2,5-dihydro-pyrazolo[4,3-c]quinolin-4-one (200 mg, 0.629 mmol) in DMSO (6 mL) was added sodium hydride (63 mg, 60% dispersion in oil, 1.57 mmol). This was allowed to stir at room temperature for about 30 minutes, at which time gas evolution had ceased and the mixture was mostly homogenous. A solution of the sulfonamide from Step 3 (320 mg, 1.57 mmol) in DMSO (0.5 mL) was added, and the reaction was stirred at RT for 5 d, at which point LCMS indicated disappearance of starting material and the presence of the desired product, in addition to the undesired O-alkyl derivative. The reaction mixture was diluted with water (50 mL) and solid NaHCO₃ was added until the reaction achieved pH 8. Solid sodium chloride was added until the solution was saturated, and the mixture was allowed to stir at rt for one hour. The resulting white precipitate was collected, rinsed with water, dried, and triturated in ethyl acetate to give a 1:1 mixture of the N- and O-alkyl products (167 mg, 60%). The isomers could also be separated by reverse phase HPLC to give the desired N-alkyl derivative as a white solid. ¹HNMR (300 MHz, CD₃OD) δ 8.86 (1H, d), 7.68 (1H, dd), 7.59 (1H, d), 6.09 (1H, dddd), 5.84 (1H, dd), 5.66 (1H, dd), 5.56 (1H, dd), 4.51 (2H, ddd), 4.18 (2H, dd), 3.94 (2H, m), 3.78 (2H, d), 2.95 (3H, s), 2.67 (2H, m), 1.81-2.34 (4 H, m), 1.61 (6H, s). LCMS: Method AA Rₜ=1.65 min, EI⁺=443.3.

### Step 5: Preparation of 5-(3-Amino-3-methyl-butyl)-8-chloro-3-methyl-2-(tetrahydro-pyran-2-yl)-2,5-dihydro-pyrazolo[4,3-c]quinolin-4-one

The allyl amine from Step 4 (518 mg, 1.17 mmol) was stirred with diethyl amine (0.484 mL, 4.68 mmol), sodium bicarbonate (196 mg, 2.34 mmol) and palladium tetrakis(triphenyl phosphine) (67 mg, 0.86 mmol) in acetonitrile at reflux for 6 h, at which point LCMS indicated disappearance of starting material and the appearance of the desired primary amine. The reaction was cooled to rt and water (50 mL) was added. The resulting tan solid was collected by filtration. Sodium chloride was added to the solution, which was then extracted with chloroform (5×25 mL). The combined organic fractions and the tan solid were combined, and the solvent removed in vacuo. The reaction was then purified by reverse phase HPLC to give the title compound as a white solid (27 mg, 6%). ¹HNMR (300 MHz, CD₃OD) δ 8.83 (1H, dd), 7.68 (1H, dd), 7.58 (1H, dd), 5.80 (1H, dd), 4.51 (2H, dd), 4.19 (2H, dd), 3.89-3.98 (2H, m), 2.94 (3H, s), 2.63-2.69 (2H, m), 1.81-2.83 (4 H, m), 1.58 (6H, s). LCMS: Method AA Rₜ=1.52 min, EI⁺=403.2.

### Step 6: Preparation of 5-(3-Amino-3-methyl-butyl)-8-chloro-3-methyl-2,5-dihydro-pyrazolo[4,3-c]quinolin-4-one

Acidic deprotection as in Example 202, Step 7 provided the title compound as a white solid. ¹HNMR (300 MHz, CD₃OD) δ 8.21 (1H, d), 7.73 (1H, dd), 7.64 (1H, d), 4.53 (2H, ddd), 2.81 (3H, s), 2.16 (2H, ddd), 1.64 (6H, s). LCMS: Method AA Rₜ=1.08 min, EI⁺=319.2.

### EXAMPLE 230

### Preparation of 8-Chloro-3-methyl-5-(2-oxo-2-pyrrolidin-2-yl-ethyl)-2,5-dihydro-pyrazolo[4,3-c]quinolin-4-one

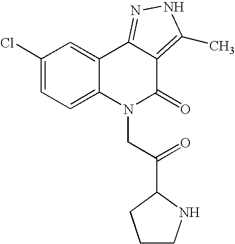

The title compound was prepared by methods outlined in Example 202 using Boc protected 2-Chloro-1-pyrrolidin-2-yl-ethanone in the alkylation step. ¹H NMR (300 MHz, DMSO-d₆) δ 9.67 (bs, 1H), 8.83 (bs, 1H), 8.24 (d, 1H), 7.54-7.64 (m, 2H), 5.30-5.57 (AB× multiplet, 2H), 4.82-4.92 (m, 1H), 3.18-3.32 (m, 2H), 2.60 (s, 3H), 2.48-2.58 (m, 2H), 2.19-2.31 (m, 1H), 1.87-2.08 (m, 2H).

### EXAMPLE 231

### Preparation of 8-Chloro-3-methyl-5-(3-methylamino-propyl)-2,5-dihydro-pyrazolo[4,3-c]quinolin-4-one

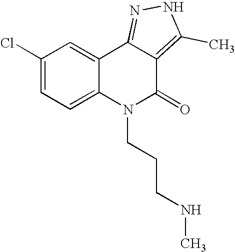

Title compound was prepared from {3-[8-chloro-3-methyl-4-oxo-2-(tetrahydro-pyran-2-yl)-2,4-dihydropyrazolo[4,3-c]quinolin-5-yl]-propyl}-carbamic acid tert-butyl ester obtained using the procedure outlined in Example 202.

### Step 1: Preparation of {3-[8-chloro-3-methyl-4-oxo-2-(tetrahydro-pyran-2-yl)-2,4-dihydropyrazolo[4,3-c]quinolin-5-yl]-propyl}-methyl-carbamic acid tert-butyl ester

To a solution of {3-[8-chloro-3-methyl-4-oxo-2-(tetrahydro-pyran-2-yl)-2,4-dihydropyrazolo[4,3-c]quinolin-5-yl]-propyl}-carbamic acid tert-butyl ester (0.23 g, 0.49 mmol) in anhyd. DMF (10 mL) was added NaH (60% dispersion, 0.028 g, 0.70 mmol) at 0° C., and the mixture was stirred for 30 min. MeI (0.035 mL, 0.56 mmol) was added and the solution was stirred for 1 h before water was added. The mixture was extracted with CH₂Cl₂. The organic layer was separated, dried over MgSO₄ and concentrated. Purification by C-18 RP LC-MS chromatography afforded {3-[8-chloro-3-methyl-4-oxo-2-(tetrahydro-pyran-2-yl)-2,4-dihydropyrazolo[4,3-c]quinolin-5-yl]-propyl}-methyl-carbamic acid tert-butyl ester (0.064 g, 27%). Purification by ISCO chromatography or recrystallization were also performed depending on the scale of the reaction. LCMS: Method FA, Rₜ=1.84 min, [MH⁺=489.2].

### Step 2: Preparation of 8-Chloro-3-methyl-5-(3-methylamino-propyl)-2,5-dihydro-pyrazolo[4,3-c]quinolin-4-one

To a solution of {3-[8-chloro-3-methyl-4-oxo-2-(tetrahydro-pyran-2-yl)-2,4 dihydropyrazolo[4,3-c]quinolin-5-yl]-propyl}-methyl-carbamic acid tert-butyl ester (0.064 g, 0.13 mmol) in 1 mL of MeOH was added 2.5 mL of HCl (4M in dioxane). The reaction was stirred for 12 h at 25° C. The reaction mixture was concentrated to afforded the title compound as a white solid (0.045 g, 100%). LCMS: Method FA, Rₜ=0.90 min, [MH⁺=305.2]. ¹H NMR 300 MHz (CD₃OD) δ 8.12-8.14 (dd, 1H), 7.57-7.59 (m, 2H), 4.40-4.46 (t, 2H), 3.00-3.07 (t, 2H), 2.71 (s, 3H), 2.66 (s, 3H), 2.07-2.19 (m, 2H).

### EXAMPLE 232

### Preparation of 5-(3-Amino-propyl)-8-fluoro-3-(2-methoxy-ethyl)-2,5-dihydro-pyrazolo[4,3-c]quinolin-4-one

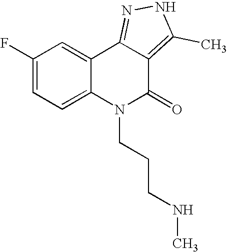

The title compound was prepared in an analogous manner to Example 231.

{3-[8-Fluoro-3-methyl-4-oxo-2-(tetrahydro-pyran-2-yl)-2,4-dihydro-pyrazolo[4,3-c]quinolin-5-yl]-propyl}-methyl-carbamic acid tert-butyl ester: To a solution of {3-[8-Fluoro-3-methyl-4-oxo-2-(tetrahydro-pyran-2-yl)-2,4-dihydro-pyrazolo[4,3-c]quinolin-5-yl]-propyl}-carbamic acid tert-butyl ester (158 mg, 0.345 mmol) in DMF was added NaH (36.0 mg, 0.900 mmol) and the mixture was stirred for 10 min before CH₃I (43.0 uL, 0.689 mmol) was added. After stirring for 18 h, the mixture was partitioned between H₂O and ethyl acetate, the aqueous layer was extracted with ethyl acetate (2×) and the combined organic layers were washed with H₂O (2×) and saturated NaCl solution before being dried over MgSO₄, filtered and concentrated in vacuo. Reverse phase chromatography on a C18 column eluting with a gradient of 60 to 100% CH₃CN in H₂O with 0.1% ammonium acetate afforded 96.0 mg (59%) of the title compound as a white solid. ¹H NMR (300 MHz, DMSO-d₆) δ 7.79 (dd, J=1.8, 8.5 Hz, 1H), 7.55 (dd, J=4.0, 9.5 Hz, 1H), 7.41 (ddd, J=1.5, 8.0, 8.0 Hz, 1H), 5.68 (d, J=8.5 Hz, 1H), 4.21 (br s, 2H), 3.89-3.98 (m, 1H), 3.66-3.78 (m, 1H), 3.18-3.29 (m, 1H), 2.75 (s, 3H), 2.32-2.46 (m, 1H), 1.90-2.11 (m, 3H), 1.70-1.89 (m, 3H), 1.54-1.65 (m, 3H), 1.33-1.48 (m, 2H), 1.16-1.31 (s, 9H); ¹⁹F NMR (282 MHz, DMSO-d₆) −121.58 to −121.47; LC/MS: AA standard Rₜ=2.26 min, EI⁺ 473.29.

### EXAMPLE 223

### Preparation of 3-Methyl-5-(3-methylamino-propyl)-2,5-dihydro-pyrazolo[4,3-c]quinolin-4-one

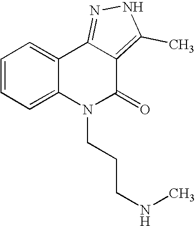

The title compound was prepared by methods described in Example 231.

Silica gel chromatography eluting with a gradient of 0 to 50% EtOAc in hexanes afforded methyl-{3-[3-methyl-4-oxo-2-(tetrahydro-pyran-2-yl)-2,4-dihydro-pyrazolo[4,3-c]quinolin-5-yl]-propyl}carbamic acid tert-butyl ester (33%). LCMS: Method FA, Rₜ=2.17 min, [MH⁺=455.3].

The HCl salt of the product: LCMS: Method FA, Rₜ=0.80 min, [MH⁺=271.2]. ¹H NMR 300 MHz (CD₃OD) δ 8.08-8.15 (m, 1H), 7.60-7.70 (m, 2H), 7.33-7.41 (m, 1H), 4.46 (t, 2H), 3.07 (t, 2H), 2.73 (s, 3H), 2.69 (s, 3H), 2.11-2.23 (m, 2H).

### EXAMPLE 234

### Preparation of 8-Iodo-3-methyl-5-(3-methylamino-propyl)-2,5-dihydro-pyrazolo[4,3-c]quinolin-4-one

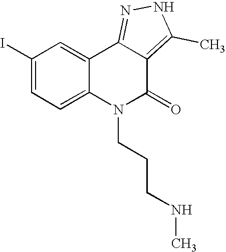

The title compound was prepared by methods described in Example 231. LCMS: Method AA, Rₜ=1.45 min, [MH⁺=397.0]. ¹H NMR 300 MHz (DMSO-d₆) δ 8.48 (d, 1H), 7.85 (dd, 1H), 7.47 (d, 1H), 4.30 (t, 2H), 2.96 (bs, 2H), 2.58 (s, 3H), 2.54 (t, 2H), 2.50 (s, 3H), 1.90-2.02 (m, 2H).

### EXAMPLE 235

### Preparation of 3,8-Dimethyl-5-(3-methylamino-propyl)-2,5-dihydro-pyrazolo[4,3-c]quinolin-4-one

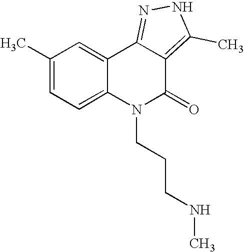

The title compound was prepared by methods described in Example 231. ¹H NMR (300 MHz, DMSO) δ 8.05 (1H, d), 7.68 (1H, s), 7.66 (1H, d), 4.58 (2H, t), 3.22 (2H, t), 2.87 (3H, s), 2.86 (3H, s), 2.60 (3H, s), 2.31 (2H, ddd). LCMS (AA) Rₜ1.07 min ES⁺=285.2 (M+1).

### EXAMPLE 236

### Preparation of 3-(2-Methoxy-ethyl)-8-methyl-5-(3-methylamino-propyl)-2,5-dihydro-pyrazolo[4,3-c]quinolin-4-one

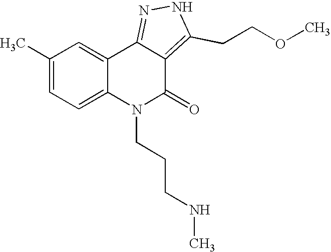

Prepared from the appropriate reagents by an analogous procedure to Example 231.

LC/MS: FA, Rₜ0.79, ES⁺=329.28 (M+1). ¹H NMR (300 MHz, CDCl3) δ 8.12 (1 H, s), 7.75-6.64 (2 H, m), 4.63 (2 H, t), 3.46 (5 H, m), 3.98 (2 H, t), 3.22 (2 H, t), 2.90 (3 H, s), 2.63 (3 H, s), 2.34 (2 H, m).

### EXAMPLE 237

### Preparation of 8-Chloro-3-(2-methoxy-ethyl)-5-(3-methylamino-propyl)-2,5-dihydro-pyrazolo[4,3-c]quinolin-4-one

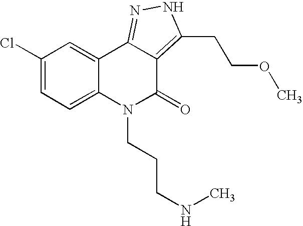

The title compound was prepared by methods described in Example 231:

{3-[8-chloro-3-(2-methoxy-ethyl)-4-oxo-2-(tetrahydro-pyran-2-yl)-2,4-dihydro-pyrazolo[4,3-c]quinolin-5-yl]-propyl}-methyl-carbamic acid tert-butyl ester (35%). LCMS: Method PFA, Rₜ=1.92 min, [MH⁺=533.3].

The HCl salt of the product: LCMS: Method FA, Rₜ=0.91 min, [MH⁺=349.1]. ¹H NMR 300 MHz (CD₃OD) δ 8.17 (s, 1H), 7.55-7.67 (m, 2H), 4.46 (t, 2H), 3.81 (t, 2H), 3.35-3.40 (m, 2H), 3.35 (s, 3H), 3.06 (t, 2H), 3.73 (s, 3H), 2.10-2.21 (m, 2H). LCMS: Method FA, Rₜ=0.91 min, [MH⁺=349.1].

### EXAMPLE 238

### Preparation of 8-Chloro-5-(3-ethylamino-propyl)-3-methyl-2,5-dihydro-pyrazolo[4,3-c]quinolin-4-one

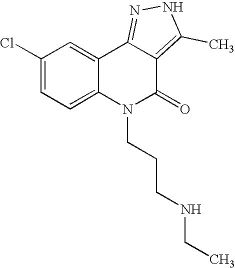

Using the appropriate reagents (bromoethane) in a manner similar to that exemplified in Example 231, the title compound was obtained as a white solid.

{3-[8-chloro-3-methyl-4-oxo-2-(tetrahydro-pyran-2-yl)-2,4-dihydro-pyrazolo[4,3-c]quinolin-5-yl]-propyl}-ethyl-carbamic acid tert-butyl ester (28%). LCMS: Method PFA, Rₜ=2.09 min, [MH⁺=503.3].

The HCl salt of the product: LCMS: Method FA, Rₜ=0.94 min, [MH⁺=319.1]. ¹H NMR 300 MHz (DMSO-d₆) δ 8.60 (bs, 1H), 8.19 (s, 1H), 7.58-7.72 (m, 2H), 4.34 (t, 2H), 2.85-3.04 (m, 4H), 2.59 (s, 3H), 1.90-2.05 (m, 2H), 1.13-1.21 (t, 3H). LCMS: Method FA, Rₜ=0.94 min, [MH⁺=319.1].

### EXAMPLE 239

### Preparation of 8-Chloro-3-methyl-5-(3-propylamino-propyl)-2,5-dihydro-pyrazolo[4,3-c]quinolin-4-one

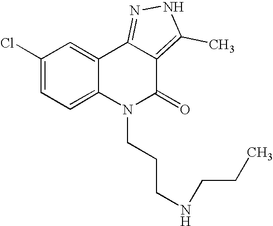

Using the appropriate reagents (1-bromopropane) in a manner similar to that exemplified in Example 231, the title compound was obtained as a white solid. {3-[8-chloro-3-methyl-4-oxo-2-(tetrahydro-pyran-2-yl)-2,4-dihydro-pyrazolo[4,3-c]quinolin-5-yl]-propyl}propyl-carbamic acid tert-butyl ester (27%). LCMS: Method AA, Rₜ=2.26 min, [MH⁺=517.3].

The HCl salt of the product: LCMS: Method FA, Rₜ=0.96 min, [MH⁺=333.6]. ¹H NMR 300 MHz (DMSO-d₆) δ 8.66 (bs, 1H), 8.20 (d, 1H), 7.70 (bs, 1H), 7.59-7.75 (m, 2H), 4.34 (t, 2H), 4.16-4.29 (m, 2H), 2.91-3.05 (m, 2H), 2.78-2.89 (m, 2H), 2.59 (s, 3H), 1.92-2.06 (m, 2H), 1.55-1.66 (t, 3H).

### EXAMPLE 240

### Preparation of 8-Chloro-5-(3-isobutylamino-propyl)-3-methyl-2,5-dihydro-pyrazolo[4,3-c]quinolin-4-one

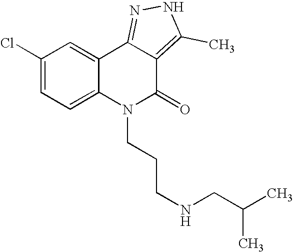

The title compound was prepared from Example 32.

2-Methyl-propionaldehyde (0.10 mL, 1.1 mmol) and magnesium sulfate (0.156 g, 1.30 mmol) were added to a solution of 5-(3-amino-propyl)-8-chloro-3-methyl-2,5-dihydro-pyrazolo[4,3-c]quinolin-4-one (0.35 g, 1.2 mmol) in CH₃OH and the mixture was stirred at 25° C. for 30 min. The mixture was filtered, and NaBH₄ (0.068 g, 1.8 mmol) reaction was partitioned between saturated aqueous NaHCO₃ and CH₂Cl₂, and the combined organic layers were dried over MgSO₄, filtered, and concentrated in vacuo. Silica gel chromatography eluting with a gradient of 0 to 10% CH₃OH in CH₂Cl₂ HCl salt by addition of HCl (4M in dioxane) to a suspension of the compound in CH₃OH followed by removal of the solvents in vacuo. LCMS: Method AA, Rₜ=1.31 min, [MH⁺=347.2]. ¹H NMR 300 MHz (DMSO-d₆) δ 8.30 (bs, 2H), 8.17 (s, 1H), 7.57-7.70 (m, 2H), 4.32 (t, 2H), 2.89-3.00 (m, 2H), 2.65-2.76 (m, 2H), 2.56 (s, 3H), 1.92-2.06 (m, 2H), 1.82-1.92 (m, 1H), 0.91 (d, 6).

### EXAMPLE 241

### Preparation of 8-Chloro-3-methyl-5-{3-[1-methyl-1H-pyrrol-2-ylmethyl)-amino]-propyl}-2,5-dihydro-pyrazolo[4,3-c]quinolin-4-one

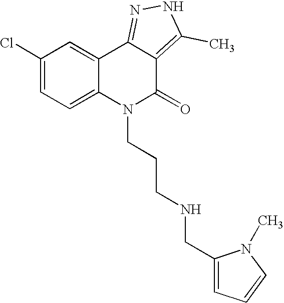

The title compound was prepared from Example 32 as described in Example 240 using 1-Methyl-1H-pyrrole-2-carbaldehyde. LCMS: Method AA, Rₜ=1.92 min, [MH⁺=384.2]. ¹H NMR 300 MHz (DMSO-d₆) δ 8.98 (bs, 2H), 8.21 (d, 1H), 7.68 (d, 1H), 7.59 (dd, 1H), 6.77 (dd, 1H), 6.29 (dd, 1H), 5.96 (dd, 1H), 4.33 (t, 2H), 4.11 (t, 2H), 3.65 (s, 3H), 2.93-3.05 (m, 2H), 2.58 (s, 3H), 2.50-2.58 (m, 2H), 1.98-2.10 (m, 2H).

### EXAMPLE 242

### Preparation of 8-Chloro-3-methyl-5-{3-[(thiophen-2-ylmethyl)-amino]-propyl}-2,5-dihydro-pyrazolo[4,3-c]quinolin-4-one

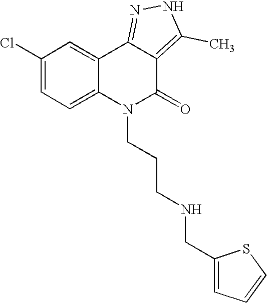

The title compound was prepared from Example 32 as described in Example 240 using thiophene-2-carbaldehyde. LCMS: Method AA, Rₜ=1.35 min, [MH⁺=387.1]. ¹H NMR 300 MHz (DMSO-d₆) δ 8.99 (bs, 2H), 8.18 (s, 1H), 7.58-7.68 (m, 3H), 7.25 (dd, 1H), 7.07 (dd, 1H), 4.28-4.40 (m, 4H), 2.94-3.05 (m, 2H), 2.58 (s, 3H), 1.95-2.05 (m, 2H).

### EXAMPLE 243

### Preparation of 8-Chloro-3-methyl-5-{3-[(1H-pyrrol-2-ylmethyl)-amino]-propyl}2,5-dihydro-pyrazolo[4,3-c]quinolin-4-one

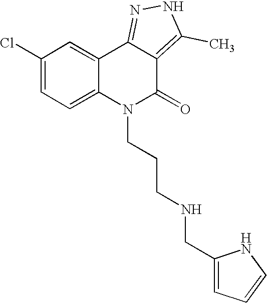

The title compound was prepared from Example 32 as described in Example 240 using 1H-Pyrrole-2-carbaldehyde. LCMS: Method PAA, Rₜ=1.87 min, [MH⁺=370.1]. ¹H NMR 300 MHz (DMSO-d₆) δ 10.55 (bs, 1H), 8.12 (d, 1H), 7.63 (d, 1H), 7.56 (dd, 1H), 6.60 (m, 1H), 5.85 (dd, 1H), 5.84 (m, 1H), 4.25 (t, 2H), 3.61 (s, 2H), 2.56 (s, 3H), 1.72 (m, 2H).

### EXAMPLE 244

### Preparation of 8-Chloro-5-{3-[(1H-imidazol-2-ylmethyl)-amino]-propyl}-3-methyl-2,5-dihydro-pyrazolo[4,3-c]quinolin-4-one

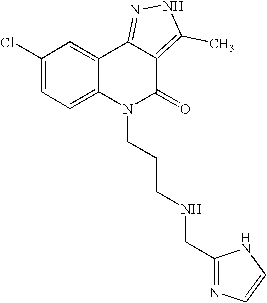

The title compound was prepared from Example 32.

1H-Imidazole-2-carbaldehyde (0.063 g, 0.66 mmol) and NaBH₃CN (0.040 mg, 1.1 mmol) were added to a solution of 5-(3-amino-propyl)-8-chloro-3-methyl-2,5-dihydro-pyrazolo[4,3-c]quinolin-4-one (0.20 g, 0.69 mmol) in CH₃OH (5 mL), and the mixture was heated at 70° C. for 18 h. After the reaction was cooled to 25° C., it was partitioned between CH₂Cl₂ and saturated aqueous NaHCO₃. The aqueous layer was extracted with CH₂Cl₂ and the combined organic layers were dried over MgSO₄, filtered, and concentrated in vacuo. Purification by C-18 RP LC-MS chromatography afforded the title compound. LCMS: Method FA, Rₜ=0.80 min, [MH⁺=370.9]. ¹H NMR 300 MHz (CD₃OD) δ 8.34 (bs, 1H), 8.13 (d, 1H), 7.57 (m, 2H), 7.14 (s, 2H), 4.43 (t, 2H), 4.18 (s, 2H), 2.98 (t, 2H), 2.67 (s, 3H), 2.03-2.15 (m, 2H).

### EXAMPLE 245

### Preparation of N-[3-(8-Chloro-3-methyl-4-oxo-2,4-dihydro-pyrazolo[4,3-c]quinolin-5-yl)-propyl]-acetamide

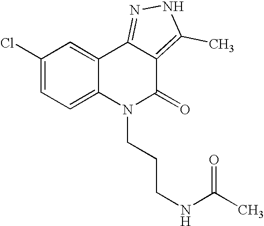

The title compound was prepared from Example 32. To a solution of 5-(3-Amino-propyl)-8-chloro-3-methyl-2,5-dihydro-pyrazolo[4,3-c]quinolin-4-one hydrochloride (200 mg, 0.611 mmol) in THF (6 mL) was added NaHMDS (1.34 mL, 1.0 M in THF, 1.34 mmol). The reaction was allowed to stir at RT until the substrate fully dissolved, about 30 minutes, before acetic anhydride (0.058 mL, 0.611 mmol) was added. The reaction was stirred at room temperature for 1 hour, at which point LCMS indicated disappearance of starting material and appearance of the desired N-acetyl compound. Water (50 mL) was carefully added, and the mixture was allowed to stir at RT for 1 hour, at which point the resulting white solid was collected, rinsed with water, and triturated in ethyl acetate to give the title compound as a white solid (55 mg, 27%). ¹HNMR (300 MHz, DMSO) δ 13.7 (1H, bs), 8.12 (1H, m), 7.88 (1H, t), 7.57 (1H, s), 4.22 (2H, t), 3.12 (2H, dd), 2.56 (3H, s), 1.80 (3H, s), 1.71 (2H, dddd). LCMS: Method AA Rₜ=1.19 min, EI⁺=319.2.

### EXAMPLE 246

### Preparation of 8-Chloro-5-(2-hydroxy-2-pyrrolidin-2-yl-ethyl)-3-methyl-2,5-dihydro-pyrazolo[4,3-c]quinolin-4-one

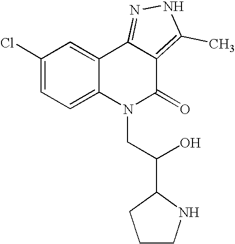

The title compound was prepared from the intermediate from Example 230.

To a solution of 2-{2-[8-Chloro-3-methyl-4-oxo-2-(tetrahydro-pyran-2-yl)-2,4-dihydro-pyrazolo[4,3-c]quinolin-5-yl]-acetyl}-pyrrolidine-1-carboxylic acid tert-butyl ester (150 mgs, 0.28 mmol) in EtOH (3 mL) and THF (few drops) at 0° C was added to NaBH₄ (13 mg, 0.34 mmol). The reaction was allowed to warm to RT over 2 hours then NH₄Cl was added. The volatiles were evaporated and EtOAc was added. The organic layer was separated and washed with brine, dried (MgSO₄), filtered and concentrated. ISCO chromatography afforded the alcohol in 70% yield. Acidic deprotection using 4N HCl and dioxane provided the title compound which was triturated using ether to afford a white solid. ¹H NMR (300 MHz, (CD₃OD)) δ 8.13 (d, 1H), 7.70 (d, 1H), 7.56 (dd, 1H), 4.59-4.66 (m, 1H), 4.10-4.26 (m, 2H), 3.62-3.76 (m, 3H), 2.68 (s, 3H), 1.91-2.18 (m, 4H).

### EXAMPLE 247

### Preparation of 8-Chloro-5-(2-hydroxy-3-methylamino-propyl)-3-methyl-2,5-dihydro-pyrazolo[4,3-c]quinolin-4-one

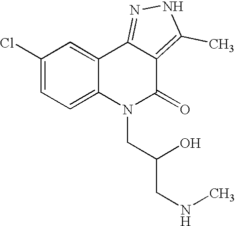

The title compound was prepared from an intermediate (8-Chloro-3-methyl-2-(tetrahydro-pyran-2-yl)-2,5-dihydro-pyrazolo[4,3-c]quinolin-4-one) synthesized by methods similar to those outline in Example 202.

### Step 1: Preparation of 8-Chloro-3-methyl-5-oxiranylmethyl-2-(tetrahydro-pyran-2-yl)-2,5-dihydro-pyrazolo[4,3-c]quinolin-4-one

To a solution of 8-Chloro-3-methyl-2-(tetrahydro-pyran-2-yl)-2,5-dihydro-pyrazolo[4,3-c]quinolin-4-one (100 mgs, 0.32 mmol) in DMF (3 mL) was added Cs₂CO₃ (521 mgs, 1.6 mmol) and epichlorohydrin (50 uL, 0.64 mmol). The reaction was stirred overnight at RT. The mixture was filtered and water was added to the filtrate. The resulting precipitate (epoxide) was filtered, dried and used without further purification.

### Step 2: Preparation of 8-Chloro-5-(2-hydroxy-3-methylamino-propyl)-3-methyl-2-(tetrahydro-pyran-2-yl)-2,5-dihydro-pyrazolo[4,3-c]quinolin-4-one

The epoxide from Step 1 was dissolved in DCM (5 mL) and excess N-methyl amine (in THF) was added. After 2 days the reaction mixture was concentrated to give an oil which was used without purification.

### Step 3: Preparation of 8-Chloro-5-(2-hydroxy-3-methylamino-propyl)-3-methyl-2,5-dihydro-pyrazolo[4,3-c]quinolin-4-one

The amino alcohol from Step 2 was dissolved in DCM (3 mL) and a few drops of 4N HCl in dioxane was added. After 1 hour, the reaction mixture was concentrated. Purification by HPLC to afford the title compound as a white solid. ¹H NMR 300 MHz (DMSO) δ 8.33 (s, 1H), 8.13 (d, 1H), 7.72 (d, 1H), 7.57 (dd, 1H), 4.38 (dd, 1H), 4.02-4.19 (overlapping m, 2H), 2.75-2.87 (m, 2H), 2.57 (s, 3H), 2.42 (s, 3H).

### EXAMPLE 248

### Preparation of 8-Chloro-5-(4-ethylamino-3-hydroxy-butyl)-3-methyl-2,5-dihydro-pyrazolo[4,3-c]quinolin-4-one

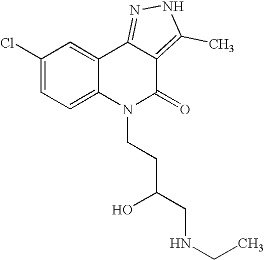

The title compound was prepared from an intermediate (5-But-3-enyl-8-chloro-3-methyl-2-(tetrahydro-pyran-2-yl)-2,5-dihydro-pyrazolo[4,3-c]quinolin-4-one) in the synthesis of Example 210.

### Step 1: Preparation of 8-Chloro-3-methyl-5-(2-oxiranyl-ethyl)-2-(tetrahydro-pyran-2-yl)-2,5-dihydro-pyrazolo[4,3-c]quinolin-4-one

To a solution of 5-But-3-enyl-8-chloro-3-methyl-2-(tetrahydro-pyran-2-yl)-2,5-dihydropyrazolo[4,3-c]quinolin-4-one (600 mg, 1.6 mmol) in DCM (20 mL) at 0° C. was added mCPBA (552 mg, 3.2 mmol). The reaction mixture gradually warmed to RT overnight. The reaction did not reach completion overnight. The reaction mixture was cooled to 0° C., and additional mCPBA was added (1 eq). After 4 h, the reaction mixture was diluted with DCM washed with Na₂SO₃ (10%), NaHCO₃ and brine. The organic layer was dried (MgSO₄), filtered and concentrated to give the crude epoxide which was purified by ISCO chromatography.

### Step 2: Preparation of 8-Chloro-5-(4-ethylamino-3-hydroxy-butyl)-3-methyl-2-(tetrahydro-pyran-2-yl)-2,5-dihydro-pyrazolo[4,3-c]quinolin-4-one

To a solution of the epoxide from Step 1 in DCM (2 mL) was added excess (10 eq) ethyl amine. After 24 h the reaction had not reached completion. Additional ethyl amine (10 eq) was added and the reaction was sealed and heated to 50° C. After 14 h, the reaction was concentrated completely.

### Step 3: Preparation of 8-Chloro-5-(4-ethylamino-3-hydroxy-butyl)-3-methyl-2,5-dihydro-pyrazolo[4,3-c]quinolin-4-one

The crude amino alcohol from Step 2 was dissolved in DCM (2 mL) and a few drops of 4N HCl in dioxane was added. After 1 hour, the reaction mixture was concentrated completely and purified by HPLC. ¹H NMR 300 MHz (DMSO) δ 8.30 (s, 1H), 8.13 (d, 1H), 7.56-7.64 (m, 2H), 4.24-4.33 (bm, 2H), 3.71-3.80 (bm, 1H), 2.62-2.79 (overlapping m, 4H), 2.56 (s, 3H), 1.59-1.81 (m, 2H), 1.08 (t, 3H).

### EXAMPLE 249

### Preparation of 8-Chloro-5-(3-hydroxy-4-methylamino-butyl)-3-methyl-2,5-dihydro-pyrazolo[4,3-c]quinolin-4-one

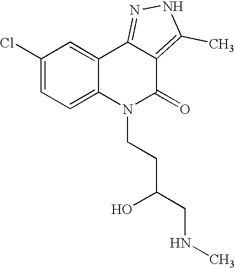

The title compound was prepared from by methods described in Example 248, using methyl amine to form the amino alcohol. ¹H NMR 300 MHz (DMSO) δ 8.36 (s, 1H), 8.18 (s, 1H), 7.65 (s, 1H), 4.31-4.39 (bm, 2H), 3.79-3.88 (m, 1H), 2.68-2.84 (m, 2H), 2.62 (s, 3H), 2.46 (s, 3H), 1.63-1.84 (m, 2H).

### EXAMPLE 250

### Preparation of 8-Chloro-5-(3-hydroxy-4-propylamino-butyl)-3-methyl-2,5-dihydro-pyrazolo[4,3-c]quinolin-4-one

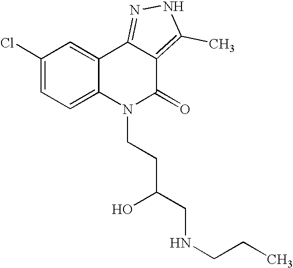

The title compound was prepared from by methods described in Example 248, using propyl amine to form the amino alcohol. ¹H NMR 300 MHz (DMSO) δ 8.59 (bs, 1H), 8.23 (s, 1H), 7.63-7.70 (m, 2H), 4.33-4.42 (m, 2H), 3.89-4.00 (m, 1H), 3.04-3.13 (m, 1H), 2.82-2.94 (overlapping m, 3H), 2.62 (s, 3H), 1.59-1.87 (overlapping m, 4H), 0.94 (t, 3H).

### EXAMPLE 251

### Preparation of 8-Chloro-5-(3-hydroxy-4-isopropylamino-butyl)-3-methyl-2,5-dihydro-pyrazolo[4,3-c]quinolin-4-one

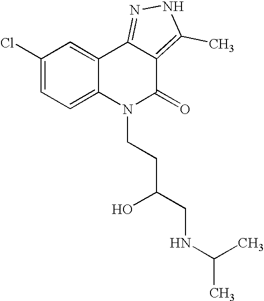

The title compound was prepared from by methods described in Example 248, using isopropyl amine to form the amino alcohol. ¹H NMR 300 MHz (DMSO) δ 8.36 (s, 1H), 8.19 (d, 1H), 7.62-7.71 (m, 2H), 4.31-4.40 (bm, 2H), 3.73-3.83 (bm, 1H), 2.93-3.03 (m, 1H), 2.64-2.83 (m, 2H), 2.62 (s, 3H), 1.63-1.86 (m, 2H), 1.12 (s, 6H).

### EXAMPLE 252

### Preparation of 8-Chloro-5-(3-hydroxy-4-imidazol-1-yl-butyl)-3-methyl-2,5-dihydro-pyrazolo[4,3-c]quinolin-4-one

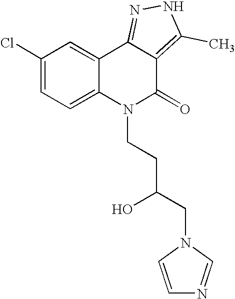

The title compound was prepared from by methods described in Example 248, using imidazole to open the epoxide. ¹H NMR 300 MHz (DMSO) δ 8.15-8.19 (m, 1H), 8.12 (s, 1H), 7.51-7.61 (bm, 3H), 7.12 (s, 1H), 6.82 (s, 1H), 4.24-4.33 (bm, 2H), 3.77-4.07 (m, 3H), 2.55 (s, 3H), 1.48-1.72 (m, 2H).

### EXAMPLE 253

### Preparation of N-[3-(8-Chloro-3-methyl-4-oxo-2,4-dihydro-pyrazolo[4,3-c]quinolin-5-yl)-propyl]-guanidine

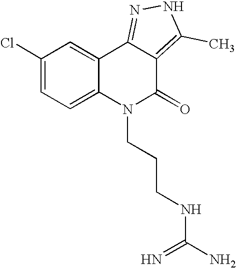

The title compound was prepared from Example 32.

### Step 1: Preparation of N-[3-(8-Chloro-3-methyl-4-oxo-2,4-dihydro-pyrazolo[4,3-c]quinolin-5-yl)-propyl]-guanidine

To a solution of 5-(3-amino-propyl)-8-chloro-3-methyl-2,5-dihydro-pyrazolo[4,3-c]quinolin-4-one hydrochloride salt (0.46 g, 1.42 mmol) and (tert-butoxycarbonylamino-trifluoromethanesulfonylimino-methyl)-carbamic acid tert-butyl ester (0.55 g, 1.42 mmol) in CH₂Cl₂ (25 mL) was added i-Pr₂NEt (1.97 mL, 11.33 mmol) and the mixture was stirred for 14 h at 22° C. The reaction mixture was concentrated in vacuo and purified by silica gel chromatography, eluting the product with a gradient of 0 to 100% EtOAc in hexanes to provide 0.25 g (0.47 mmol) of the Boc-protected guanidine adduct in 33% yield. LCMS: Method FA, Rₜ=2.54 min, [MH⁺=533.3].

### Step 2: Preparation of N-[3-(8-Chloro-3-methyl-4-oxo-2,4-dihydro-pyrazolo[4,3-c]quinolin-5-yl)-propyl]-guanidine

The HCl salt of the title compound was prepared after deprotection as described in Example 202, Step 7. Purification by C-18 RP LC-MS chromatography provided the HCOOH salt. ¹H NMR 300 MHz (DMSO) δ 8.79-8.91 (m, 1H), 8.46 (s, 1H), 8.14 (s, 1H), 7.73-7.90 (bm, 2H), 7.61-7.73 (m, 1H), 7.49-7.60 (m, 1H), 4.16-4.32 (m, 2H), 3.25-3.55 (m, 4H), 3.07-3.24 (m, 2H), 2.58 (s, 3H), 1.71-1.88 (m, 2H). LCMS: Method FA, Rₜ=1.41 min, [MH⁺=333.1].

### EXAMPLE 254

### Preparation of N″-13-(8-Chloro-3-methyl-4-oxo-2,4-dihydro-pyrazolo[4,3-c]quinolin-5-yl)-propyl]-N,N,N′,N′-tetramethyl-guanidine

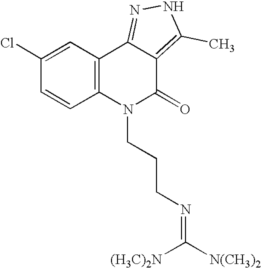

The title compound was prepared from Example 32.

To a solution of 5-(3-amino-propyl)-8-chloro-3-methyl-2,5-dihydro-pyrazolo[4,3-c]quinolin-4-one (0.19 g, 0.66 mmol) and HATU (0.31 g, 0.83 mmol) in CH₂Cl₂ (20 mL) was added i-Pr₂NEt (0.23 mL, 1.32 mmol) and the mixture was stirred for 24 h at 22° C. The reaction mixture was concentrated in vacuo and purified by C-18 RP LC-MS chromatography to provide 0.11 g (0.26 mmol) of the title compound as the HCOOH salt in 39% yield. ¹H NMR 300 MHz (DMSO) δ 8.10 (s, 1H), 7.48-7.65 (m, 3H), 4.14-4.35 (m, 2H), 3.06-3.22 (m, 2H), 2.68-3.03 (bm, 12H), 2.51 (s, 3H), 1.74-1.97 (m, 2H). LCMS: Method FA, Rₜ=1.05 min, [MH⁺=389.4].

### EXAMPLE 255

### Preparation of 8-(3-Amino-propoxy)-5-(3-amino-propyl)-3-methyl-2,5-dihydro-pyrazolo[4,3-c]quinolin-4-one

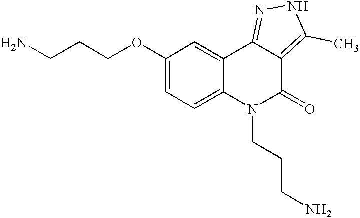

### Step 1: Preparation of 6-(tert-Butyl-dimethyl-silanyloxy)-1H-benzo[d][1,3]oxazine-2,4-dione

tert-Butyldimethylsilyl chloride (1.0M in CH₂Cl₂, 40 ML, 40 mmol) and TEA (8.0 mL, 58 mmol) were added to a solution of 5-hydroxy isatoic anhydride (5.0 g, 28 mmol) in DMF (200 mL) at 0° C. The mixture was gradually warmed up to 25° C. and stirred for 18 hr. The reaction mixture was diluted with CH₂Cl₂. The organic layer was washed with H₂O, dried over MgSO₄, filtered, and concentrated. The residue was triturated with hexanes to give 5.8 g (70%) of the title product. ¹H NMR 300 MHz (CDCl₃) δ 9.67 (bs, 1H), 7.46 (d, 1H), 7.19 (dd, 1H), 7.04 (d, 1H), 0.97 (s, 9H), 0.20 (s, 6H).

### Step 2: Preparation of 2-Amino-5-(tert-butyl-dimethyl-silanyloxy)-benzoic acid methyl ester

The methyl ester was formed using a method similar to Example 208, Step 2.

¹H NMR 300 MHz (CDCl₃) δ 7.35 (d, 1H), 6.89 (dd, 1H), 6.62 (d, 1H), 3.90 (s, 3H), 1.01 (s, 9H), 0.20 (s, 6H).

### Step 3: 5-(tert-Butyl-dimethyl-silanyloxy)-2-(3-oxo-butyrylamino)-benzoic acid methyl ester

The methyl ester from Step 2 was treated in an analogous manner as in Example 202-Step 2 using the appropriate reagents to provide the title compound. LCMS: Method FA, Rₜ=2.29 min, [MH⁺=366.2].

### Step 4: Preparation of 3-Acetyl-6-(tert-butyl-dimethyl-silanyloxy)-4-hydroxy-1H-quinolin-2-one

Sodium methoxide (0.64 g, 12 mmol) was added to a solution of 5-(tert-butyl-dimethyl-silanyloxy)-2-(3-oxo-butyrylamino)-benzoic acid methyl ester (4.3 g, 12 mmol) in CH₃OH (50 mL). The mixture was refluxed for 18 h, then cooled to 25° C.

The yellow precipitate formed after the reaction mixture was diluted with saturated aqueous NH₄Cl was collected and dried under vacuum to give 3.1 g (79%) of the title product. LCMS: Method FA, Rₜ=2.35 min, [MH⁺=334.2].

### Step 5: Preparation of 8-Hydroxy-3-methyl-2,5-dihydro-pyrazolo[4,3-c]quinolin-4-one

The title compound was prepared in a similar manner to Example 202, Step 4. LCMS: Method AA, Rₜ=0.89 min, [MH⁺=216.0].

### Step 6: Preparation of 8-(tert-Butyl-diphenyl-silanyloxy)-3-methyl-2,5-dihydro-pyrazolo[4,3-c]quinolin-4-one

tert-Butyldiphenylsilyl chloride (2.9 mL, 11 mmol) and imidazole (1.21 g, 18 mmol) were added to a solution of 8-hydroxy-3-methyl-2,5-dihydro-pyrazolo[4,3-c]quinolin-4-one (1.97 g, 9.2 mmol) in DMF (50 mL). The mixture was stirred at 25° C. for 18 h. The yellow precipitate formed after the reaction mixture was diluted with H₂O was collected and dried under vacuum to give 4.1 g (99%) of the title product. LCMS: Method AA, Rₜ=2.35 min, [MH⁺=454.2].

### Step 7: Preparation of 8-(tert-Butyl-diphenyl-silanyloxy)-3-methyl-2-(tetrahydro-pyran-2-yl)-2,5-dihydro-pyrazolo[4,3-c]quinolin-4-one

3,4-Dihydro-2H-pyran (1.15 mL, 12.7 mmol) and p-toluenesulfonic acid monohydrate (0.08 g, 0.4 mmol) were added to a solution of 8-(tert-butyl-diphenyl-silanyloxy)-3-methyl-2,5-dihydro-pyrazolo[4,3-c]quinolin-4-one (1.2 g, 2.6 mmol) in DMF (20 mL). The mixture was stirred at 60° C. for 24 h then cooled to 25° C. The white precipitate formed after the reaction mixture was diluted with Et₂O was collected and dried under vacuum to give 1.03 g (74%) of the title product. LCMS: Method AA, Rₜ=2.61 min, [MH⁺=538.3].

### Step 8: Preparation of {3-[3-Methyl-4-oxo-2-(tetrahydro-pyran-2-yl)-4,5-dihydro-2H-pyrazolo[4,3-c]quinolin-8-yloxy]-propyl}carbamic acid tert-butyl ester

Cesium carbonate (0.725 g, 2.23 mmol) and (3-bromo-propyl)-carbamic acid tert-butyl ester (0.125 g, 0.525 mmol) were added to a solution of 8-(tert-butyl-diphenyl-silanyloxy)-3-methyl-2-(tetrahydro-pyra-2-yl)-2,5-dihydro-pyrazolo[4,3-c]quinolin-4-one (0.24 g, 0.45 mmol) in DMF (5 mL). The mixture was stirred at 25° C. for 18 h. The reaction mixture was diluted with CH₂Cl₂. The organic layer was washed with H₂O, dried over MgSO₄, filtered, and concentrated. The crude product was used without further purification. LCMS: Method AA, Rₜ=1.73 min, [MH⁺=457.2].

### Step 9: Preparation of 8-(3-Amino-propoxy)-5-(3-amino-propyl)-3-methyl-2,5-dihydro-pyrazolo[4,3-c]quinolin-4-one

The title compound was prepared using methods outline in Example 202, Step 7. The product was purified by HPLC. LCMS: Method FA, Rₜ=0.44 min, [MH⁺=330.2]. ¹H NMR 300 MHz (CD₃OD) δ 7.92 (bs, 2H), 7.84 (bs, 2H), 7.76 (s, 1H), 7.58 (d, 1H), 7.19 (d, 1H), 4.32 (t, 2H), 4.17 (t, 2H), 2.95-3.05 (m, 2H), 2.80-2.91 (m, 2H), 2.58 (s, 3H), 2.02-2.13 (m, 2H), 1.87-2.00 (m, 2H).

### EXAMPLE 256

### Preparation of 5-(3-Amino-propyl)-8-ethoxy-3methyl-2,5-dihydro-pyrazolo[4,3-c]quinolin-4-one

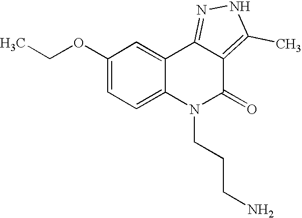

### Step 1: Preparation of 8-Ethoxy-3-methyl-2-(tetrahydro-pyran-2-yl)-2,5-dihydro-pyrazolo[4,3-c]quinolin-4-one

The title compound was prepared from the intermediate from Step 7 in Example 255.

Potassium tert-butoxide (0.054 g, 0.48 mmol) was added to a solution of 8-(tert-butyl-diphenyl-silanyloxy)-3-methyl-2-(tetrahydro-pyran-2-yl)-2,5-dihydro-pyrazolo[4,3-c]quinolin-4-one (0.20 g, 0.44 mmol) in DMF (5 mL). The mixture was stirred for 5 min. Bromoethane (0.036 mL, 0.48 mmol) and potassium carbonate (0.068 g, 0.49 mmol) were added, and the mixture was stirred at 25° C. for 18 h. The white precipitate formed after the reaction mixture was diluted with H₂O was collected and dried under vacuum. The crude product was triturated with hexane to give 0.107 g (74%) of the title product. LCMS: Method FA, Rₜ=1.61 min, [MH⁺=328.1].

### Step 2: Preparation of 5-(3-Amino-propyl)-8-ethoxy-3methyl-2,5-dihydro-pyrazolo[4,3-c]quinolin-4-one

The title compound was prepared as in Example 255, Step 9. LCMS: Method FA, Rₜ0.89 min, [MH⁺=301.2]. ¹H NMR 300 MHz (DMSO-d₆) δ 7.82 (bs, 3H), 7.67 (d, 1H), 7.51 (d, 1H), 7.12 (dd, 1H), 4.26 (t, 2H), 4.07 (q, 2H), 2.75-2.84 (m, 2H), 2.52 (s, 3H), 1.83-1.94 (m, 2H), 1.33 (t, 3H).

### EXAMPLE 257

### Preparation of 5-(3-Amino-propyl)-8-benzyloxy-3-methyl-2,5-dihydro-pyrazolo[4,3-c]quinolin-4-one

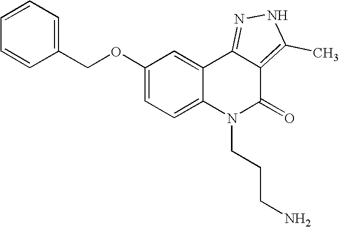

The title compound was prepared as in Example 256.

LCMS: Method FA, Rₜ=1.06 min, [MH⁺=363.2]. ¹H NMR 300 MHz (DMSO-d₆) δ 7.77 (bs, 2H), 7.76 (s, 1H), 7.28-7.55 (m, 6H), 7.21 (dd, 1H), 5.16 (s, 2H), 4.26 (q, 2H), 2.76-2.85 (m, 2H), 2.52 (s, 3H), 1.84-1.95 (m, 2H).

### EXAMPLE 258

### Preparation of 5-(3-Amino-propyl)-3-methyl-8-propoxy-2,5-dihydro-pyrazolo[4,3-c]quinolin-4-one

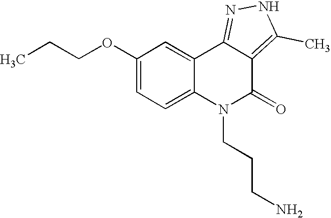

The title compound was prepared as in Example 256.

LCMS: Method FA, Rₜ=1.01 min, [MH⁺=315.2]. ¹H NMR 300 MHz (DMSO-d₆) δ 7.86 (bs, 3H), 7.72 (d, 1H), 7.56 (d, 1H), 7.17 (dd, 1H), 4.31 (t, 2H), 4.02 (t, 2H), 2.81-2.90 (m, 2H), 2.56 (s, 3H), 1.87-1.97 (m, 2H), 1.78 (q, 2H), 1.01 (t, 3H).

### EXAMPLE 259

### Preparation of 5-(3-Amino-propyl)-8-isobutoxy-3-methyl-2,5-dihydro-pyrazolo[4,3-c]quinolin-4-one

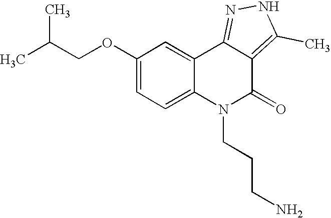

The title compound was prepared as in Example 256.

LCMS: Method FA, Rₜ=1.08 min, [MH⁺=329.2]. ¹H NMR 300 MHz (DMSO-d₆) δ 7.80 (bs, 3H), 7.66 (d, 1H), 7.50 (d, 1H), 7.12 (dd, 1H), 4.25 (t, 2H), 3.77 (d, 2H), 2.74-2.84 (m, 2H), 2.50 (s, 3H), 1.96-2.01 (m, 1H), 1.81-1.93 (m, 2H), 0.96 (d, 6H).

### EXAMPLE 260

### Preparation of 8-Amino-5-(3-amino-propyl)-3-methyl-2,5-dihydro-pyrazolo[4,3-c]quinolin-4-one

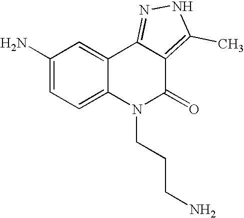

The title compound was prepared from {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester an intermediate obtained via methods outlined in Example 202.

{3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester: To a solution of {3-[3-Methyl-8-nitro-4-oxo-2-(tetrahydro-pyran-2-yl)-2,4-dihydro-pyrazolo[4,3-c]quinolin-5-yl]-propyl}-carbamic acid tert-butyl ester (112 mg, 0.231 mmol) in CH₃OH was added 10 weight % palladium on activated carbon (13.3 mg) and the mixture was stirred under H₂ atmosphere for 4 h before the mixture was filtered. Chromatography eluting with 0 to 10% CH₃OH in CH₂Cl₂ afforded 96.1 mg (92%) of the title compound as a grey solid. ¹H NMR (300 MHz, DMSO-d₆) δ 7.31-7.41 (m, 1H), 7.19 (d, J=8.9 Hz, 1H), 6.75-6.96 (m, 2H), 5.63 (d, J=9.0 Hz, 1H), 5.14 (s, 2H), 4.06-4.23 (m, 2H), 3.92 (d, J=11.2 Hz, 1H), 3.64-3.79 (m, 1H), 2.92-3.10 (m, 2H), 2.72 (s, 3H), 2.31-2.47 (m, 1H), 1.89-2.15 (m, 2H), 1.64-1.84 (m, 3H), 1.53-1.64 (m, 2H), 1.38 (s, 9H); LC/MS: AA standard Rₜ=1.63 min, EI⁺ 456.29.

Acidic deprotection afforded the title compound: ¹H NMR (300 MHz, DMSO-d₆) δ 10.36 (br s, 2H), 8.14 (d, J=2.4 Hz, 1H), 8.05 (br s, 2H), 7.77 (d, J=9.1 Hz, 1H), 7.59 (dd, J=2.4, 9.0 Hz, 1H), 4.34 (t, J=6.7 Hz, 2H), 2.81-2.94 (m, 2H), 2.61 (s, 3H), 1.90-2.03 (m, 2H); LC/MS: AA standard Rₜ=0.77 min, EI⁺ 270.26.

### EXAMPLE 261

### Preparation of N-[5-(3-Amino-propyl)-3-methyl-4-oxo-4,5-dihydro-2H-pyrazolo[4,3-c]quinolin-8-yl]-acetamide

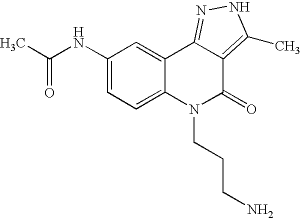

The title compound was prepared from {3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 260.

To a solution of {3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester (101 mg, 0.222 mmol) in CH₂Cl₂ was added triethylamine (80.0 uL, 0.574 mmol) and the solution was stirred 10 min before acetyl chloride (20.0 uL, 0.281 mmol) was added and the solution stirred overnight. Chromatography eluting with 0 to 10% CH₃OH in CH₂Cl₂ afforded 22.6 mg (21%) of the title compound as a white solid. ¹H NMR (300 MHz, DMSO-d₆) δ 10.09 (s, 1H), 8.45 (d, J=2.3 Hz, 1H), 7.69 (dd, J=2.3, 9.1 Hz, 1H), 7.44 (d, J=9.2 Hz, 1H), 6.90 (t, J=4.9 Hz, 1H), 5.63-5.70 (m, 1H), 4.15-4.23 (m, 2H), 3.89-3.98 (m, 1H), 3.64-3.78 (m, 1H), 2.97-3.07 (m, 2H), 2.74 (s, 3H), 2.34-2.46 (m, 1H), 1.91-2.13 (m, 2H), 2.07 (s, 3H), 1.53-1.80 (m, 5H), 1.39 (s, 9H); LC/MS: AA standard Rₜ1.66 min, EI⁺ 498.27.

Acidic deprotection as outline above afforded the title compound: ¹H NMR (300 MHz, DMSO-d₆) δ 10.21 (s, 1H), 8.46 (d, J=1.8 Hz, 1H), 7.93 (br s, 3H), 7.53-7.71 (m, 2H), 4.30 (t, J=6.1 Hz, 2H), 2.80-2.93 (m, 2H), 2.59 (s, 3H), 2.08 (s, 3H), 1.89-2.01 (m, 2H); LC/MS: AA standard Rₜ=0.93 min, EI⁺ 314.18.

### EXAMPLE 262

### Preparation of Pyrrolidine-2S-carboxylic acid [5-(3-amino-propyl)-3-methyl-4-oxo-4,5-dihydro-2H-pyrazolo[4,3-c]quinolin-8-yl]-amide

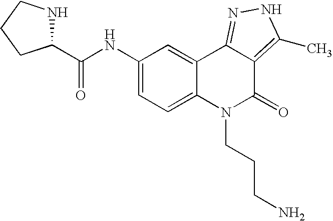

The title compound was prepared from {3-[8-Amino-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 260 by methods outlined in Example 335. ¹H NMR (300 MHz, DMSO-d₆) δ 11.1 (s, 1H), 10.1 (br s, 1H), 8.70 (br s, 1H), 8.43-8.55 (m, 1H), 8.01 (br s, 3H), 7.58-7.84 (m, 2H), 4.39-4.55 (m, 1H), 4.24-4.39 (m, 2H), 3.22-3.38 (m, 2H), 2.79-2.99 (m, 2H), 2.68 (s, 1H), 2.60 (s, 3H), 1.87-2.10 (m, 5H) ppm; LC/MS: AA standard Rₜ=0.88 min, EI⁻ 367.48.

### EXAMPLE 263

### Preparation of Pyrrolidine-2R-carboxylic acid [5-(3-amino-propyl)-3-methyl-4-oxo-4,5-dihydro-2H-pyrazolo[4,3-c]quinolin-8-yl]-amide

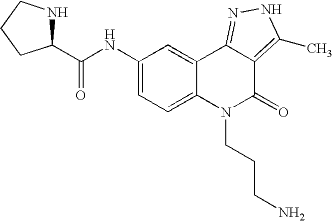

The title compound was prepared by analogous methods to Example 262. ¹H NMR (300 MHz, DMSO-d₆) δ 11.2 (s, 1H), 10.1 (br s, 1H), 8.71 (br s, 1H), 8.46-8.53 (m, 1H), 8.01 (br s, 3H), 7.76 (dd, J=2.2, 9.1 Hz, 1H), 7.65 (d, J=9.3 Hz, 1H), 4.39-4.52 (m, 1H), 4.32 (t, J=5.6 Hz, 2H), 3.22-3.37 (m, 2H), 2.82-2.94 (m, 2H), 2.69 (s, 1H), 2.60 (s, 3H), 1.90-2.06 (m, 5H) ppm; LC/MS: AA standard Rₜ=0.88 min, EI⁻ 367.48.

### EXAMPLE 264

### Preparation of N-[5-(3-aminopropyl)-3-methyl-4-oxo4,5-dihydro-2H-pyrazolo[4,3-c]quinolin-8-yl]-2-dimethylaminoacetamide

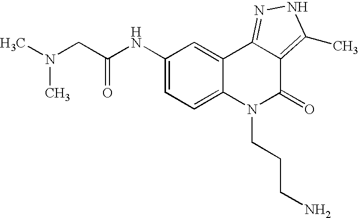

The title compound was prepared by analogous methods to Example 262. ¹H NMR (300 MHz, DMSO-d₆) δ 11.1 (s, 1H), 10.1 (br s, 1H), 8.48 (s, 1H), 7.96 (br s, 2H), 7.61-7.78 (m, 2H), 4.27-4.38 (m, 2H), 4.18-4.26 (m, 2H), 2.91 (s, 6H), 2.81-2.89 (m, 2H), 2.60 (s, 3H), 1.89-2.04 (m, 2H) ppm; LC/MS: AA standard Rₜ=0.98 min, EI⁺ 357.26.

### EXAMPLE 265

### Preparation of N-[5-(3-aminopropyl)-3-methyl-4-oxo-4,5-dihydro-2H-pyrazolo[4,3-c]quinolin-8-yl]-2-dimethylamino-propionamide

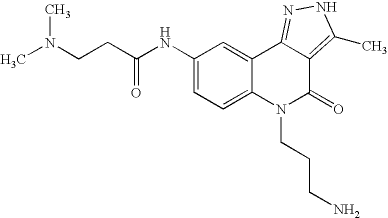

The title compound was prepared by analogous methods to Example 262.

### EXAMPLE 266

### Preparation of 5-(3-Amino-propyl)-3-methyl-4-oxo-4,5-dihydro-2H-pyrazolo[4,3-c]quinoline-8-carboxylic acid

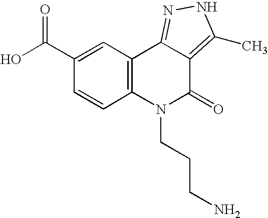

The title compound was prepared from {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate in Example 208.

### Step 1: Preparation of 5-(3-tert-butoxycarbonylamino-propyl)-3-methyl-4-oxo-2-(tetrahydro-pyran-2-yl)-4,5-dihydro-2H-pyrazolo[4,3-c]quinoline-8-carboxylic acid

To a solution of {3-[8-iodo-3-methyl-4-oxo-2-(tetrahydro-pyran-2-yl)-2,4-dihydro-pyrazolo[4,3-c]quinolin-5-yl]-propyl}-carbamic acid tert-butyl ester (0.44 g, 0.78 mmol) in dry DMF (3 mL) was added Ac₂O (0.15 mL, 1.55 mmol), HCOOLi (0.12 g, 2.33 mmol), LiCl (0.99 g, 2.33 mmol), and Pd(OAc)₂ (0.01 g, 0.039 mmol). Lastly, i-Pr₂NEt (0.27 mL, 1.55 mmol) and the mixture heated for 16 h at 80° C. in a sealed tube. The reaction mixture was then diluted with CH₂Cl₂ (30 mL), and the organic layer was washed with H₂O (3×30 mL), dried over MgSO₄, filtered and concentrated in vacuo to give crude product as a yellow solid. The solid was treated with EtOAc and sonicated to provide a white solid which was collected by filtration to provide 0.25 g (0.52 mmol) of 5-(3-tert-butoxycarbonylamino-propyl)-3-methyl-4-oxo-2-(tetrahydro-pyran-2-yl)-4,5-dihydro-2H-pyrazolo[4,3-c]quinoline-8-carboxylic acid in 67% yield. LCMS: Method FA, Rₜ=1.75 min, [MH⁺=485.3].

### Step 2: Preparation of 5-(3-Amino-propyl)-3-methyl-4-oxo-4,5-dihydro-2H-pyrazolo[4,3-c]quinoline-8-carboxylic acid

The HCl salt of the title compound was prepared after deprotection as described in Example 202, Step 7. ¹H NMR 300 MHz (DMSO) δ 8.81 (s, 1H), 8.06-8.15 (m, 1H), 7.85-8.01 (bm, 2H), 7.74-7.81 (m, 1H), 4.27-4.47 (m, 2H), 2.83-3.02 (m, 2H), 2.62 (m, 3H), 1.87-2.09 (m, 2H). LCMS: Method FA, Rₜ=0.81 min, [MH⁺=301.2].

### EXAMPLE 267

### Preparation of 3-[5-(3-Amino-propyl)-3-methyl-4-oxo-4,5-dihydro-2H-pyrazolo[4,3-c]quinolin-8-yl]-acrylic acid methyl ester

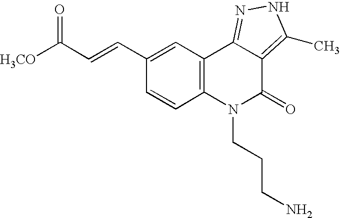

The title compound was prepared from {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate in Example 208.

To a solution of K₂CO₃ (0.17 g, 1.25 mmol) and n-Bu₄NCl (0.14 g, 0.5 mmol) in DMF (7 mL) and H₂O (0.7 mL) was added PPh₃ (0.13 g, 0.05 mmol), methyl acrylate (0.90 mL, 1.00 mmol), and {3-[8-iodo-3-methyl-4-oxo-2-(tetrahydro-pyran-2-yl)-2,4-dihydro-pyrazolo[4,3-c]quinolin-5-yl]-propyl}-carbamic acid tert-butyl ester (0.28 g, 0.5 mmol) and the mixture stirred for 15 min at 22° C. Finally, added Pd(OAc)₂ (0.006 g, 0.025 mmol) and the mixture was heated for 2 h at 50° C. in a sealed tube. The reaction mixture was then diluted with EtOAc (30 mL), and the organic layer was washed with H₂O (3×30 mL), dried over MgSO₄, filtered and concentrated in vacuo to give crude product as a brown solid which was purified by silica gel chromatography, eluting the product with a gradient of 0 to 100% EtOAc in hexanes to provide 0.20 g (0.38 mmol) of 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-acrylic acid methyl ester in 75% yield. LCMS: Method FA, Rₜ=2.13 min, [MH⁺=525.3].

The HCl salt of the title compound was prepared after deprotection as described in Example 202, Step 7. ¹H NMR 300 MHz (DMSO) δ 8.45-8.54 (m, 1H), 7.60-7.98 (bm, 5H), 6.68 (d, 1H), 4.27-4.42 (m, 2H), 3.74 (s, 3H), 2.79-2.96 (m, 2H), 2.57 (s, 3H), 1.83-2.00 (m, 2H). LCMS: Method FA, Rₜ=0.93 min, [MH⁺=341.2].

### EXAMPLE 268

### Preparation of 3-[5-(3-Amino-propyl)-3-methyl-4-oxo-4,5-dihydro-2H-pyrazolo[4,3-c]quinolin-8-yl]-propionic acid methyl ester

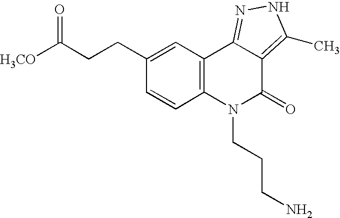

The title compound was prepared from 3-[5-(3-tert-Butoxycarbonylamino-propyl)-3-methyl-4-oxo-2-(tetrahydro-pyran-2-yl)-4,5-dihydro-2H-pyrazolo[4,3-c]quinolin-8-yl]-acrylic acid methyl ester, an intermediate in Example 267.

To a solution of 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-acrylic acid methyl ester (0.11 g, 0.21 mmol) in a 2/1 mixture of MeOH/EtOAc (5 mL) was added Pd/C (10% wt, ˜50% H₂O, 0.02 g) and the mixture was placed under H₂ (1 atm) and stirred for 72 h at 22° C. The reaction mixture was filtered through celite and the filtrate concentrated in vacuo to give crude product as a white solid which was purified by silica gel chromatography, eluting the product with a gradient of 0 to 100% EtOAc in hexanes to provide 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-propionic acid methyl ester. LCMS: Method FA, Rₜ=2.06 min, [MH⁺=527.3].

The HCl salt of the title compound was prepared after deprotection as described in Example 202, Step 7 to give 0.03 g (0.06 mmol) of product for 17% yield. ¹H NMR 300 MHz (DMSO) δ 8.02 (s, 1H), 7.73-7.92 (bm, 2H), 7.52-7.60 (m, 1H), 7.42-7.51 (m, 1H), 4.28-4.38 (m, 2H), 3.60 (s, 3H), 2.92-3.02 (m, 2H), 2.82-2.92 (m, 1H), 2.68-2.77 (m, 2H), 2.58 (s, 3H), 1.87-2.00 (m, 2H). LCMS: Method FA, Rₜ=0.91 min, [MH⁺=343.2].

### EXAMPLE 269

### Preparation of 3-[5-(3-Aminopropyl)-3-methyl-4-oxo-4,5-dihydro-2H-pyrazolo[4,3-c]quinolin-8-yl]-propionic acid

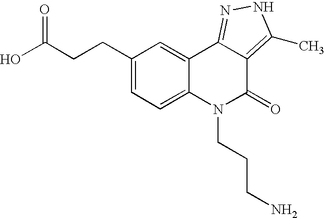

The title compound was prepared from Example 268.

To a solution of 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-propionic acid methyl ester (0.20 g, 0.38 mmol) in THF (10 mL) was added 3 mL of 1 M NaOH solution and the mixture was stirred for 12 h at 22° C. The reaction mixture was treated with 3 mL 1 N HCl, diluted with 3 mL H₂O, and the aqueous layer extracted with CH₂Cl₂ (15 mL) followed by EtOAc (15 mL). The combined organic layers were dried over MgSO₄, filtered and concentrated to give 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-propionic acid as a white solid. LCMS: Method FA, Rₜ=1.74 min, [MH⁺=513.3].

The HCl salt of the title compound was prepared after deprotection as described in Example 202, Step 7 to give 0.042 g (0.13 mmol) of product. ¹H NMR 300 MHz (DMSO) δ 7.94-8.14 (bm, 3H), 7.51-7.60 (m, 1H), 7.39-7.50 (m, 1H), 4.23-4.39 (m, 2H), 2.75-3.00 (m, 4H), 2.60-2.68 (m, 2H), 2.57 (s, 3H), 1.86-2.04 (m, 2H). LCMS: Method FA, Rₜ=0.85 min, [MH⁺=329.2].

### EXAMPLE 270

### Preparation of 2-Acetylamino-3-[5-(3-amino-propyl)-3-methyl-4-oxo-4,5-dihydro-2H-pyrazolo[4,3-c]quinolin-8-yl]-acrylic acid methyl ester

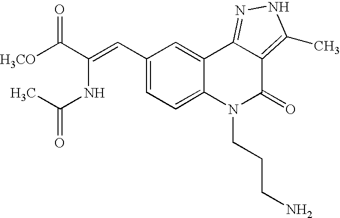

The title compound was prepared from {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate in Example 208.

To a solution of K₂CO₃ (0.092 g, 0.67 mmol) and n-Bu₄NCl (0.074 g, 0.27 mmol) in DMF (1 mL) and H₂O (0.1 mL) was added PPh₃ (0.007 g, 0.027 mmol), 2-acetylamino-acrylic acid methyl ester (0.076 mg, 0.53 mmol), and {3-[8-iodo-3-methyl-4-oxo-2-(tetrahydro-pyran-2-yl)-2,4-dihydro-pyrazolo[4,3-c]quinolin-5-yl]-propyl}-carbamic acid tert-butyl ester (0.15 g, 0.27 mmol) and the mixture stirred for 15 min at 22° C. Finally, added Pd(OAc)₂ (0.003 g, 0.013 mmol) and the mixture was heated for 100 s at 150° C. in a Personal Chemistry Smith Creator microwave. The reaction mixture was then diluted with EtOAc (10 mL), and the organic layer was washed with H₂O (3×10 mL), dried over MgSO₄, filtered and concentrated in vacuo to give crude product as a brown solid which was purified by C-18 RP LC-MS chromatography to provide 0.027 g (0.046 mmol) of 2-acetylamino-3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-acrylic acid methyl ester in 17% yield. LCMS: Method FA, Rₜ=1.72 min, [MH⁺=582.3].

The HCl salt of the title compound was prepared after deprotection as described in Example 202, Step 7 0.016 g (0.041 mmol) of product for 88% yield. ¹H NMR 300 MHz (DMSO) δ 9.69-9.80 (m, 1H), 8.40-8.53 (m, 1H), 7.71-8.00 (m, 3H), 7.62-7.70 (m, 1H), 7.27 (m, 1H), 4.26-4.41 (m, 2H), 3.73 (s, 3H), 2.80-2.96 (m, 2H), 2.59 (s, 3H), 2.04 (m, 3H), 1.86-2.01 (m, 2H). LCMS: Method FA, Rₜ=0.84 min, [MH⁺=398.3].

### EXAMPLE 271

### Preparation of 5-(3-Amino-propyl)-3-methyl-4-oxo-4,5-dihydro-2H-pyrazolo[4,3-c]quinoline-8-carbonitrile

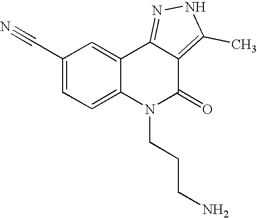

The title compound was prepared from {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 208.

### Step 1: Preparation of {3-[8-Cyano-3-methyl-4-oxo-2-(tetrahydro-pyran-2-yl)-2,4-dihydro-pyrazolo[4,3-c]quinolin-5-yl]-propyl}-carbamic acid tert-butyl ester

To a solution of {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester (250 mg, 0.441 mmol) in 4 mL anhydrous DMF at room temperature was added zinc cyanide (3 1.1 mg, 0.265 mmol), tris(dibenzylideneacetone)dipalladium (20.2 mg, 0.022 mmol), 1,1 ′-bis(diphenylphosphino)ferrocene (29.4 mg, 0.053 mmol) and a drop of water. The solution was degassed with argon then stirred at 120° C. for 16 hours. The solution was allowed to cool to room temperature then diluted with ethyl acetate and saturated aqueous sodium bicarbonate. The organic layer was then washed with water followed by brine, dried over sodium sulfate and concentrated in vacuo. The residue was purified by silica gel chromatography (ISCO, elution with 10-50% ethyl acetate in hexanes) to give 131 mg product as a white solid.

### Step 2: Preparation of 5-(3-Amino-propyl)-3-methyl-4-oxo-4,5-dihydro-2H-pyrazolo[4,3-c]quinoline-8-carbonitrile

To a solution of {3-[8-Cyano-3-methyl-4-oxo-2-(tetrahydro-pyran-2-yl)-2,4-dihydro-pyrazolo[4,3-c]quinolin-5-yl]-propyl}-carbamic acid tert-butyl ester (131 mg, 0.28 mmol) in 4 mL DCM and 1 ml MeOH at room temperature was added 2 ml 4.0 M HCl in dioxane. The solution was stirred at room temperature for 6 hours. Ether was added and the precipitate was then filtered and washed with ether to give 64.3 mg product as a white solid. LCMS: Method FA, Rₜ=0.83 min, [MH⁺=282.22]. ¹H NMR (300 MHz, D₂O) δ 7.81-7.84 (m, 1H), 7.74 (d, 1H), 7.42 (m, 1H), 4.17 (t, 2H), 3.05 (t, 2H), 2.54 (s, 3H), 1.96-2.09 (m, 2H).

### EXAMPLE 272

### Preparation of 5-(3-Amino-propyl)-8-(3-hydroxy-prop-1-ynyl)-3-methyl-2,5-dihydro-pyrazolo[4,3-c]quinolin-4-one

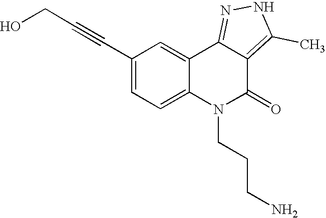

The title compound was prepared from {3-[8-Iodo-3-methyl-4-oxo-2-(tetrahydro-pyran 2-yl)-2,4-dihydro-pyrazolo[4,3-c]quinolin-5-yl]-propyl}-carbamic acid tert-butyl ester, an intermediate in Example 208.

### Step 1: Preparation of {3-[8-(3-Hydroxy-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester

To a solution of {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester (250 mg, 0.441 mmol) in 4 mL DMF at room temperature was added dichlorobis(triphenylphosphine)palladium (10.8 mg, 0.015 mmol), copper iodide (6.7 mg, 0.035 mmol), and triethylamine (0.25 mL, 1.77 mmol). The solution was degassed with argon, and stirred at room temperature for one hour. Then added propargyl alcohol (0.051 mL, 0.88 mmol) and the solution was stirred at 60° C. for 16 hours (for some alkynes the reaction was carried out at room temperature). The solution was then allowed to cool to room temperature, and was diluted with ethyl acetate and water. The organic phase was washed with water followed by brine, dried over sodium sulfate and the concentrated in vacuo. The residue was purified by silica gel chromatography (ISCO, elution with 10-50% ethyl acetate in hexanes) to give 162 mg product as a white solid.

### Step 2: Preparation of 5-(3-Amino-propyl)-8-(3-hydroxy-prop-1-ynyl)-3-methyl 2,5-dihydro-pyrazolo[4,3-c]quinolin-4-one

To a solution of {3-[8-(3-Hydroxy-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester (162 mg, 0.328 mmol) in 4 mL DCM and 1 ml MeOH at room temperature was added 2 ml 4.0 M HCl in dioxane. The solution was stirred at room temperature for 6 hours. Ether was added and the precipitate was then filtered and washed with ether to give 63.7 mg product as a white solid. LCMS: Method FA, Rₜ=0.94 min, [MH⁺=311.25]. ¹H NMR (300 MHz, D₂O) δ 7.41 (s, 1H), 7.38 (d, 1H), 7.16 (d, 2H), 4.53 (s, 2H), 4.09 (t, 2H), 3.02 (t, 2H), 2.49 (s, 3H), 1.99 (m, 2H).

### EXAMPLE 273

### Preparation of 5-(3-Amino-propyl)-8-(3-amino-prop-1-ynyl]-3-methyl 2,5-dihydro-pyrazolo[4,3-c]quinolin-4-one

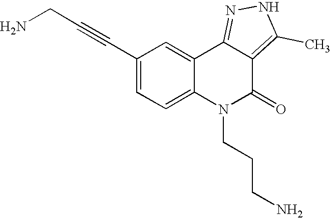

The title compound was prepared as in Example 272 using Prop-2-ynyl-carbamic acid tert-butyl ester as the coupling partner. LCMS: Method FA, Rₜ=0.72 min, [MH⁺=310.26]. ¹H NMR (300 MHz, D₂O) δ 7.77 (s, 1H), 7.57 (d, 1H), 7.31 (d, 1H), 4.19 (m, 2H), 4.15 (s, 2H), 3.01 (t, 2H), 2.54 (s, 3H), 2.02 (m, 2H).

### EXAMPLE 274

### Preparation of 5-(3-Amino-propyl)-3-methyl 8-(3-methylamino-prop-1-ynyl]-2,5-dihydro-pyrazolo[4,3-c]quinolin-4-one

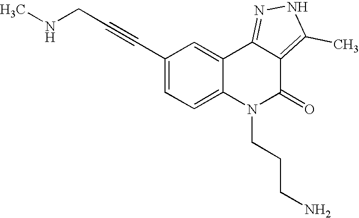

The title compound was prepared as in Example 272 using the appropriate reagents.

LCMS: Method FA, Rₜ=0.63 min, [MH⁺=324.17]. ¹H NMR (300 MHz, D₂O) δ 7.7 (s, 1H), 7.56 (d, 1H), 7.30 (d, 1H), 4.21 (s, 2H), 4.17 (t, 2H), 2.98 (t, 2H), 2.89 (s, 3H), 2.51 (s, 3H), 1.94-2.07 (m, 2H).

### EXAMPLE 275

### Preparation of 5-(3-Amino-propyl)-8-[3-(benzyl-methyl-amino)-prop-1-ynyl]-3-methyl-2,5-dihydro-pyrazolo[4,3-c]quinolin-4-one

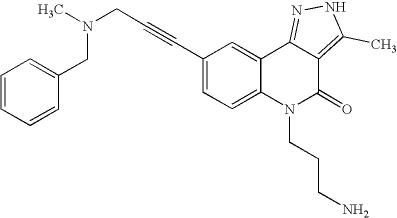

The title compound was prepared as in Example 272 using the appropriate reagents.

LCMS: Method FA, Rₜ=1.11 min, [MH⁺=414.13]. ¹H NMR (300 MHz, D₂O) δ 7.94 (s, 1H), 7.67 (d, 1H), 7.59 (m, 5H), 7.40 (d, 2H), 4.55 (m, 2H), 4.26 (m, 4H), 3.05 (s, 3H), 3.02 (m, 2H), 2.59 (s, 3H), 2.07 (m, 2H).

### EXAMPLE 276

### Preparation of N-{³-[5-(3-Amino-propyl)-3-methyl-4-oxo4,5-dihydro-2H-pyrazolo[4,3-c]quinolin-8-yl]-prop-2-ynyl]-acetamide

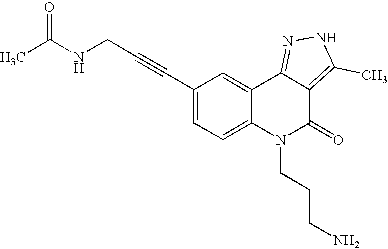

The title compound was prepared as in Example 272 using the appropriate reagents.

LCMS: Method FA, Rₜ=0.82 min, [MH⁺=352.16]. ¹H NMR (300 MHz, D₂0) δ 7.30-7.38 (m, 2H), 7.13 (d, 2H), 4.24 (s, 2H), 4.08 (t, 2H), 3.03 (t, 2H), 2.48 (s, 3H), 2.11 (s, 3H), 1.96-2.04 (m, 2H).

### EXAMPLE 277

### Preparation of 5-(3-Amino-propyl)-3-methyl-8-pyridin-3-ylethylynyl]-2,5-dihydro-pyrazolo[4,3-c]quinolin-4-one

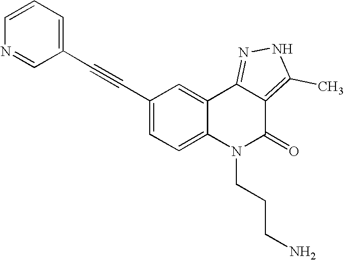

The title compound was prepared as in Example 272 using the appropriate reagents.

LCMS: Method FA, Rₜ=1.25 min, [MH⁺=358.04]. ¹H NMR (300 MHz, D₂0) δ 8.86-8.90 (m, 1H), 8.72 (d, 1H), 8.57 (d, 1H), 7.97-8.05 (m, 1H), 7.71-7.76 (m, 1H), 7.60 (d, 1H), 7.37 (d, 1H), 4.21 (t, 2H), 3.04 (t, 2H), 2.48 (s, 3H), 2.00-2.11 (m, 2H).

### EXAMPLE 278

### Preparation of 5-(3-Amino-propyl)-3-methyl-8-pyridin-2-ylethylynyl-2,5-dihydro-pyrazolo[4,3-c]quinolin-4-one

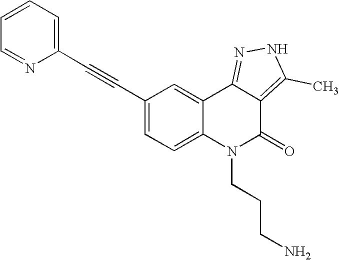

The title compound was prepared as in Example 272 using the appropriate reagents.

LCMS: Method FA, Rₜ=0.93 min, [MH⁺=358.22]. ¹H NMR (300 MHz, D₂O) δ 8.60 (d, 1H), 8.34-8.43 (m, 1H), 7.94 (d, 1H), 7.80-7.88 (m, 1H), 7.77 (s, 1H), 7,64 (d, 1H), 7.41 (d, 1H), 4.21 (t, 2H), 3.06 (t, 2H), 2.48 (s, 3H), 2.00-2.11 (m, 2H).

### EXAMPLE 279

### Preparation of 5-(3-Amino-propyl)-3-methyl-8-pyridin-4-ylethylynyl-2,5-dihydro-pyrazolo[4,3-c]quinolin-4-one

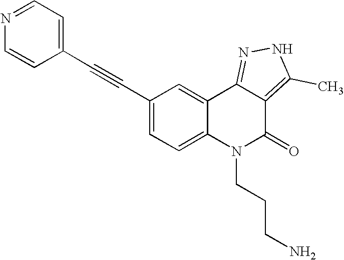

The title compound was prepared as in Example 272 using the appropriate reagents.

LCMS: Method FA, Rₜ=0.84 min, [MH⁺=358.14]. ¹H NMR (300 MHz, D₂O) δ 8.68 (d, 2H), 7.94 (d, 2H), 7.79-7.83 (m, 1H), 7.65 (d, 1H), 7.42 (d, 1H), 4.25 (t, 2H), 3.06 (t, 2H), 2.50 (s, 3H), 2.02-2.13 (m, 2H).

### EXAMPLE 280

### Preparation of 5-(3-Amino-propyl)-3-methyl-8-(3-phenoxy-prop-1-ynyl)-2,5-dihydro-pyrazolo[4,3-c]quinolin-4-one

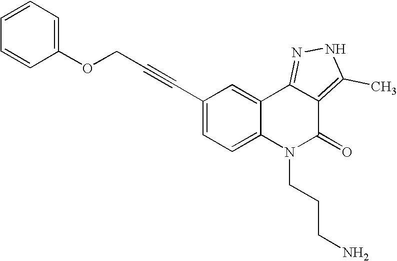

The title compound was prepared as in Example 272 using the appropriate reagents.

LCMS: Method FA, Rₜ=1.17 min, [MH⁺=387.13]. ¹H NMR (300 MHz, DMSO) δ 8.23-8.26 (m, 1H), 7.85-7.97 (m, 2H), 7.58-7.69 (m, 2H), 7.31-7.39 (m, 2H), 6.95-7.10 (m, 3H), 5.09 (s, 2H), 4.32 (t, 2H), 2.81-2.93 (m, 2H), 2.57 (s, 3H), 1.87-1.99 (m, 2H).

### EXAMPLE 281

### Preparation of 5-(3-Amino-propyl)-8-(3-methoxy-prop-1-ynyl)-3-methyl-2,5-dihydro-pyrazolo[4,3-c]quinolin-4-one

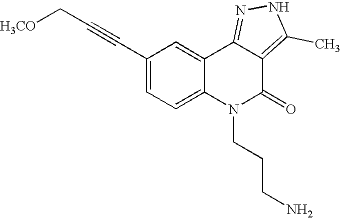

The title compound was prepared as in Example 272 using the appropriate reagents.

LCMS: Method FA, Rₜ=0.93 min, [MH⁺=325.08]. ¹H NMR (300 MHz, D₂O) δ 7.58 (s, 1H), 7.49 (s, 1H), 7.26 (d, 1H), 4.47 (s, 2H), 4.17 (t, 2H), 3.55 (s, 3H), 3.04(t, 2H), 2.54 (s, 3H), 1.98-2.08 (m, 2H).

### EXAMPLE 282

### Preparation of 5-(3-Amino-propyl)-8-(3-hydroxy-but-1-ynyl)-3-methyl 2,5-dihydro-pyrazolo[4,3-c]quinolin-4-one

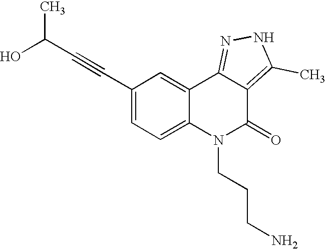

The title compound was prepared as in Example 272 using the appropriate reagents.

LCMS: Method FA, Rₜ=0.84 min, [MH⁺=325.14]. ¹H NMR (300 MHz, D₂O) δ 7.46-7.55 (m, 2H), 7.26 (d, 1H), 4.90-5.00 (m, 1H), 4.19 (t, 2H), 3.11 (t, 2H), 2.60 (s, 3H), 2.03-2.16 (m, 2H), 1.68 (d, 3H).

### EXAMPLE 283

### Preparation of 5-(3-Amino-propyl)-3-methyl 8-prop-1-ynyl-2,5-dihydro-pyrazolo[4,3-c]quinolin-4-one

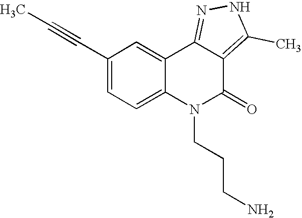

The title compound was prepared from {3-[8-Iodo-3-methyl-4-oxo-2-(tetrahydro-pyran 2-yl)-2,4-dihydro-pyrazolo[4,3-c]quinolin-5-yl]-propyl}-carbamic acid tert-butyl ester, an intermediate in Example 208.

To a solution of {3-[8-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester (200 mg, 0.353 mmol) in 4 mL DMF at room temperature was added dichlorobis(triphenyl-phosphine)palladium (8.7 mg, 0.012 mmol), copper iodide (5.4 mg, 0.028 mmol), and triethylamine (0.20 mL, 1.41 mmol). The reaction was cooled to −78° C., and propyne gas was bubbled through the solution for 15 minutes then stirred at 60° C. for 6 hours. The solution was then allowed to cool to room temperature, and was diluted with ethyl acetate and water. The organic phase was washed with water followed by brine, dried over sodium sulfate and the concentrated in vacuo. The residue was purified by silica gel chromatography (ISCO, elution with 10-50% ethyl acetate in hexanes) to give 149 mg product as a white solid.

The HCl salt of the title compound was prepared as described in Example 272, Step 2. LCMS: Method FA, Rₜ=0.93 min, [MH⁺=295.19]. ¹H NMR (300 MHz, D₂O) δ 7.27-7.34 (m, 2H), 7.11 (d, 1H), 4.08 (t, 2H), 3.03 (t, 2H), 2.49 (s, 3H), 2.10 (s, 3H), 1.93-2.04 (m, 2H).

### EXAMPLE 284

### Preparation of 5-(3-Amino-propyl)-8-(3-dimethylamino-prop-1-ynyl]-3-methyl 2,5-dihydro-pyrazolo[4,3-c]quinolin-4-one

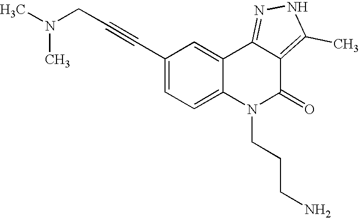

The title compound was prepared from {3-[8-Iodo-3-methyl-4-oxo-2-(tetrahydro pyran-2-yl)-2,4-dihydro-pyrazolo[4,3-c]quinolin-5-yl]-propyl}-carbamic acid tert-butyl ester, an intermediate in Example 208.

### Step 1: Preparation of Methanesulfonic acid 3-[5-(3-tert-butoxycarbonylamino-propyl)-3-methyl-4-oxo-2-(tetrahydro-pyran-2-yl)-4,5-dihydro-2H-pyrazolo[4,3-c]quinolin-8-yl]-prop-2-ynyl ester

To a solution of {3-[8-(3-Hydroxy-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester (610 mg, 1.23 mmol) in 7 mL anhydrous THF at room temperature was added triethylamine (0.39 mL, 2.77 mmol). The solution was cooled to 0° C., then methane-sulfonyl chloride (0.11 mL, 1.36 mmol) was added dropwise. The solution was stirred at 0° C. for 1 hour then the solvent was evaporated. The residue was purified by silica gel chromatography (ISCO, elution with 10-50% ethyl acetate in hexanes) to give 530 mg product as a white solid.

### Step 2: Preparation of {3-[8-(3-Dimethylamino-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester

To a solution of the mesylate (200 mg, 0.349 mmol) in 4 mL anhydrous ethyl alcohol at room temperature was added triethylamine (0.058 mL, 0.419 mmol) followed by dimethylamine (2.0 M solution in THF, 1.75 mL, 3.49 mmol). The solution was stirred at reflux for 1 hour then cooled to room temperature. The solvent was evaporated and the residue was diluted with dichloromethane and water. The organic phase was washed with water then brine, dried over sodium sulfate, then concentrated in vacuo. The residue was purified by silica gel chromatography (ISCO, elution with 10-70% ethyl acetate in hexanes) to give 124 mg product as a clear oil.

The HCl salt was prepared as described in Example 272, Step 2. LCMS: Method FA, Rₜ=0.65 min, [MH⁺=338.23]. ¹H NMR (300 MHz, D₂O) δ 7.89 (s, 1H), 7.63 (s, 1H), 7.38 (s, 1H), 4.35 (s, 2H), 4.24 (m, 2H), 3.07 (s, 6H), 3.04 (m, 2H), 2.56 (s, 3H), 2.05 (m, 2H).

### EXAMPLE 285

### Preparation of 5-(3-Amino-propyl)-3-methyl-8-(3-pyrrolidin-1-yl-prop-1-ynyl]-2,5-dihydro-pyrazolo[4,3-c]quinolin4-one

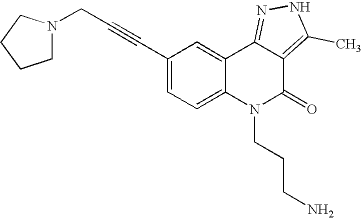

The title compound was prepared from the mesylate intermediate from Example 284.

To a solution of the mesylate (328 mg, 0.57 mmol) in 8 mL anhydrous ethyl alcohol at room temperature was added triethylamine (0.088 mL, 0.63 mmol) followed by pyrrolidine (0.053 mL, 0.63 mmol). The solution was stirred at reflux for 4 hours then cooled to room temperature. The solvent was evaporated and the residue was diluted with dichloromethane and water. The organic phase was washed with water then brine, dried over sodium sulfate, then concentrated in vacuo. The residue was purified by silica gel chromatography (ISCO, elution with 10-70% ethyl acetate in hexanes) to give 125 mg product as a clear oil.

The HCl salt was prepared as described in Example 272, Step 2. LCMS: Method FA,. Rₜ=0.72 min, [MH⁺=364.23]. ¹H NMR (300 MHz, D₂O) δ 7.94 (s, 1H), 7.65 (d, 1H), 7.42 (d, 1H), 4.39 (s, 2H), 4.29 (t, 2H), 3.58 (m, 4H), 3.05 (t, 2H), 2.6 (s, 3H), 2.18 (m, 4H), 2.09 (m, 2H).

### EXAMPLE 286

### Preparation of 5-(3-Amino-propyl)-3-methyl-8-(3-piperidin-1-yl-prop-1-ynyl]-2,5-dihydro-pyrazolo[4,3-c]quinolin-4-one

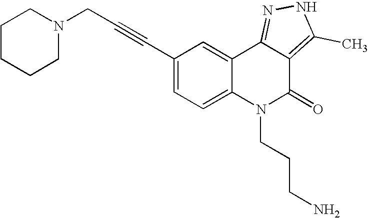

The title compound was prepared from the mesylate intermediate from Example 284.

To a solution of piperidine (0.047 mL, 0.473 mmol) and N,N-diisopropylethylamine (0.30 mL, 1.72 mmol) in 2 mL DMF at room temperature was added a solution of the mesylate (246 mg, 0.43 mmol) in 2 mL DMF dropwise. The solution was stirred at room temperature for 2 hours, then diluted with water and ethyl acetate. The organic phase was washed with water followed by brine, dried over sodium sulfate and concentrated in vacuo. The residue was purified by silica gel chromatography (ISCO, elution with 0-10% methanol in dichloromethane) to give 101 mg product as a yellow solid.

The HCl salt was prepared as described in Example 272, Step 2. LCMS: Method FA, Rₜ=0.76 min, [MH⁺=378.30]. ¹H NMR (300 MHz, D₂O) δ 8.05-8.12 (m, 1H), 7.77 (d, 1H), 7.54 (d, 1H), 4.28-4.46 (m, 4H), 3.57-3.96 (m, 2H), 3.21-3.53 (m, 2H), 3.16 (t, 2H), 2.71 (s, 3H), 1.57-2.27 (m, 8H).

### EXAMPLE 287

### Preparation of 8-[3-(4-Acetyl-piperazin-1-yl)-prop-1-ynyl]-5-(3-amino-propyl)-3-methyl-2,5-dihydro-pyrazolo[4,3-c]quinolin-4-one

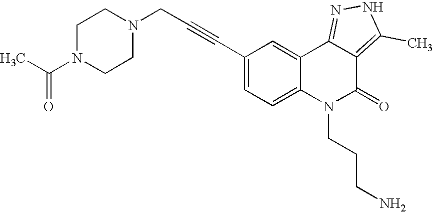

The title compound was prepared as in Example 284 using the appropriate reagents.

LCMS: Method FA, Rₜ=0.98 min, [MH⁺=421.27]. ¹H NMR (300 MHz, D₂O) δ 7.92-7.96 (m, 1H), 7.65 (d, 1H), 7.41 (d, 1H), 4.46 (m, 2H), 4.27 (t, 2H), 3.34-3.95 (m, 8H), 3.05 (t, 2H), 2.59 (s, 3H), 2.21 (s, 3H), 2.00-2.14 (m, 2H).

### EXAMPLE 288

### Preparation of 5-(3-Amino-propyl)-3-methyl-8-[3-(4-methyl-piperazin-1-yl)-prop-1-ynyl]-2,5-dihydro-pyrazolo[4,3-c]quinolin-4-one

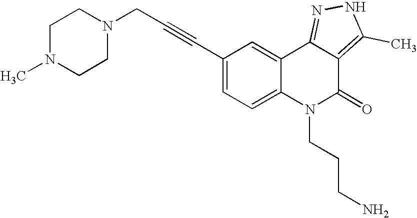

The title compound was prepared as in Example 284 using the appropriate reagents.

LCMS: Method FA, Rₜ=0.98 min, [MH⁺=393.28]. ¹H NMR (300 MHz, D₂O) δ 7.99 (s, 1H), 7.68 (d, 1H), 7.45 (d, 1H), 4.31 (t, 2H), 4.16 (s, 2H), 3.40-3.74 (br m, 8H), 3.01-3.10 (m, 5H), 2.61 (s, 3H), 2.04-2.14 (m, 2H).

### EXAMPLE 289

### Preparation of 5-(3-Amino-propyl)-3-methyl-8-(3-morpholin-4-yl-prop-1-ynyl]-2,5-dihydro-pyrazolo[4,3-c]quinolin-4-one

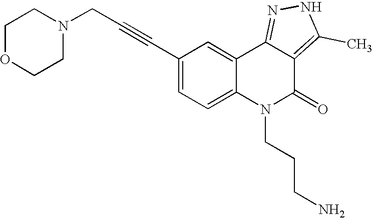

The title compound was prepared as in Example 284 using the appropriate reagents.

LCMS: Method FA, Rₜ=0.72 min, [MH⁺=380.27]. ¹H NMR (300 MHz, D₂O) δ 8.02 (s, 1H), 7.69 (d, 2H), 7.46 (d, 2H), 4.35 (s, 2H), 4.33 (t, 2H), 4.00-4.12 (m, 4H), 3.49-3.59 (m, 4H), 3.06 (t, 2H), 2.62 (s, 3H), 2.05-2.15 (m, 2H).

### EXAMPLE 290

### Preparation of 5-(3-Amino-propyl)-8-(3-imidazol-1-yl-prop-1-ynyl]-3-methyl-2,5-dihydro-pyrazolo[4,3-c]quinolin-4-one

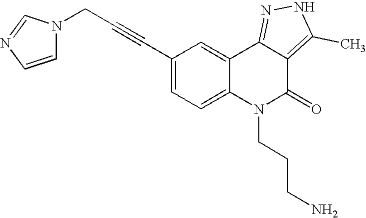

The title compound was prepared from the mesylate intermediate from Example 284.

To a solution of imidazole (46 mg, 0.672 mmol) and N,N-diisopropylethylamine (0.42 mL, 2.44 mmol) in 3 mL DMF at room temperature was added a solution of the mesylate (246 mg, 0.43 mmol) in 3 mL DMF dropwise. The solution was stirred at room temperature for 18 hours, then at 90° C. for 2 hours. The solution was cooled to room temperature, then diluted with water and ethyl acetate. The organic phase was washed with water followed by brine, dried over sodium sulfate and concentrated in vacuo. The residue was purified by silica gel chromatography (ISCO, elution with 0-10% methanol in dichloromethane) to give 89 mg product as a yellow solid.

The HCl salt was prepared as described in Example 272, Step 2. LCMS: Method FA, Rₜ=0.98 min, [MH⁺=361.25]. ¹H NMR (300 MHz, D₂O) δ 9.00 (s, 1H), 7.85 (m, 1H), 7.77 (m, 1H), 7.61 (m, 2H), 7.38 (d, 2H), 5.44 (s, 2H), 4.25 (t, 2H), 3.04 (t, 2H), 2.56 (s, 3H), 2.07 (m, 2H).

### EXAMPLE 291

### Preparation of 5-(3-Amino-propyl)-8-ethynyl-3-methyl-2,5-dihydro-pyrazolo[4,3-c]quinolin-4-one

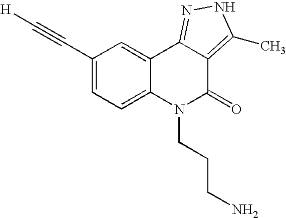

The title compound was prepared from {3-[8-Iodo-3-methyl-4-oxo-2-(tetrahydro-pyran 2-yl)-2,4-dihydro-pyrazolo[4,3-c]quinolin-5-yl]-propyl-carbamic acid tert-butyl ester, an intermediate from Example 208.

### Step 1: Preparation of {3-[3-Methyl-4-oxo-2-(tetrahydro-pyran-2-yl)-8-trimethylsilanylethynyl-2,4-dihydro-pyrazolo[4,3-c]quinolin-5-yl]-propyl}-carbamic acid tert-butyl ester

This compound was prepared from {3-[8-Iodo-3-methyl-4-oxo-2-(tetrahydro-pyran-2-yl)-2,4-dihydro-pyrazolo[4,3-c]quinolin-5-yl]-propyl-carbamic acid tert-butyl ester and Ethynyl-trimethyl-silane using the procedure described in Example 272.

### Step 2: Preparation of {3-[8-Ethynyl-3-methyl-4-oxo-2-(tetrahydro-pyran-2-yl)-2,4-dihydro-pyrazolo[4,3-c]quinolin-5-yl]-propyl}-carbamic acid tert-butyl ester

To a suspension of the TMS acetylene (700 mg, 1.30 mmol) in 13 mL MeOH at room temperature was added potassium carbonate (270 mg, 1.96 mmol). The mixture was stirred at room temperature for 16 hours at room temperature. The solvent was evaporated, then the residue was diluted with dichloromethane and water. The organic phase was washed with water then brine, dried over sodium sulfate and concentrated in vacuo. The residue was purified by silica gel chromatography (ISCO, elution with 10-40% ethyl acetate in hexanes) to give 474 mg product as a white solid.

### Step 3: Preparation of 5-(3-Amino-propyl)-8-ethynyl-3-methyl-2,5-dihydro-pyrazolo[4,3-c]quinolin-4-one

To a solution of the acetylene (2.81 g, 6.056 mmol) in MeOH (200 mL) was added concentrated aq HCl (4.0 mL). The reaction was sealed and stirred 4 days, then concentrated in vacuo to afford 1.91 g of the title compound as a white amorphous solid. LCMS: Method FA, Rₜ=0.92 min, [MH⁺=281.3]. ¹H NMR (300 MHz, D₂O) δ 7.73 (s, 1H), 7.62 (d, 1H), 7.36 (d, 1H), 4.29 (t, 2H), 3.75 (s, 1H), 3.16 (t, 2H), 2.67 (s, 3H), 2.15 (m, 2H).

### EXAMPLE 292

### Preparation of 5-(3-Amino-propyl)-8-ethynyl-3-(2-methoxy-ethyl)-2,5-dihydro-pyrazolo[4,3-c]quinolin-4-one

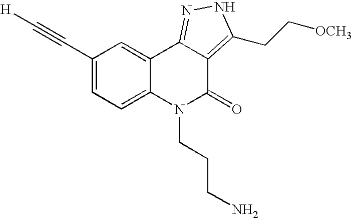

The title compound was prepared by methods outlined in Example 291.

RP HPLC eluting with CH₃CN in 0.1% aqueous NH₄OC(O)CH₃. ¹H NMR (300 MHz, DMSO-d₆) δ 8.26 (s, 1H), 7.64 (s, 2H), 4.26-4.36 (m, 2H), 4.25 (s, 1H), 3.71 (t, J=6.9 Hz, 2H), 3.25 (s, 3H), 3.22 (t, J=6.9 Hz, 2H), 2.67 (t, J=6.6 Hz, 2H), 1.84 (s, 3H), 1.74 (quintet, J=6.8 Hz, 2H) ppm; LC/MS: AA standard Rₜ=1.07 min, EI⁺ 325.19.

### EXAMPLE 293

### Preparation of 8-Ethynyl-3-methyl-5-(3-methylamino-propyl)-2,5-dihydro-pyrazolo[4,3-c]quinolin-4-one

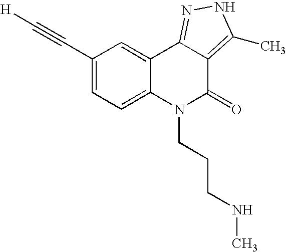

The title compound was prepared from {3-[8-Iodo-3-methyl-4-oxo-2-(tetrahydro-pyran 2-yl)-2,4-dihydro-pyrazolo[4,3-c]quinolin-5-yl]-propyl-carbamic acid tert-butyl ester, an intermediate from Example 208 using procedures outlined in Example 291 and Example 231.

LCMS: Method AA, Rₜ=1.01 min, [MH⁺=295.3]. ¹H NMR 300 MHz (CD₃OD) δ 8.41 (d, 1H), 7.84 (dd, 1H), 7.74 (d, 1H), 4.61 (t, 2H), 3.75 (s, 1H), 3.20 (t, 2H), 2.87 (s, 3H), 2.83 (s, 3H), 2.25-2.36 (m, 2H).

### EXAMPLE 294

### Preparation of 5-(3-Amino-propyl)-8-ethyl-3-methyl-2,5-dihydro-pyrazolo[4,3-c]quinolin4-one

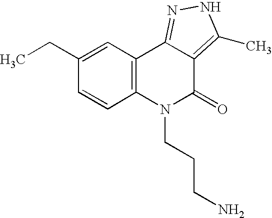

The title compound was prepared from Example 291.

To a solution of 5-(3-amino-propyl)-8-ethynyl-3-methyl-2,5-dihydro-pyrazolo[4,3-c]quinolin-4-one (0.36 g, 1.28 mmol) in a 2:1 mixture of MeOH/EtOAc (5 mL) was added Pd/C (10% wt, ˜50% H₂O, 0.04 g) and the mixture was placed under H₂ (1 atm) and stirred for 12 h at 22 ° C. The reaction mixture was filtered through celite and the filtrate concentrated in vacuo to give crude product as a yellow oil. The oil was treated with EtOAc and sonicated to provide a white solid which was collected by filtration to provide 0.20 g (0.69 mmol) of the title compound in 54% yield. ¹H NMR 300 MHz (DMSO) δ 8.01 (s, 1H), 7.52-7.59 (m, 2H), 7.39-7.51 (m, 2H), 4.26-4.39 (m, 2H), 2.65-2.79 (m, 4H), 2.59 (s, 3H), 1.71-1.86 (m, 2H), 1.29 (t, 3H). LCMS: Method PFA, Rₜ=1.32 min, [MH⁺=285.3].

### EXAMPLE 295

### Preparation of 5-(3-amino-propyl)-8-(3-methoxypropyl)-3-methyl-2,5-dihydro-pyrazolo[4,3-c]quinolin-4-one

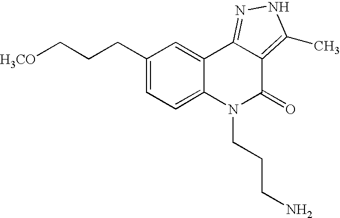

The title compound was prepared from Example 281 using methods similar to Example 294. ¹H NMR 300 MHz (DMSO) δ 8.46 (s, 1H), 8.02 (d, 1H), 7.58 (d, 1H), 7.45 (dd, 1H), 4.34 (t, 2H), 3.40 (t, 2H), 3.29 (s, 3H), 2.73-2.85 (m, 4H), 2.61 (s, 3H), 1.85-1.95 (m, 4H).

### EXAMPLE 296

### Preparation of 5-(3-Amino-propyl)-8-(3-dimethylamino-propyl)-3-methyl-2,5-dihydro-pyrazolo[4,3-c]quinolin-4-one

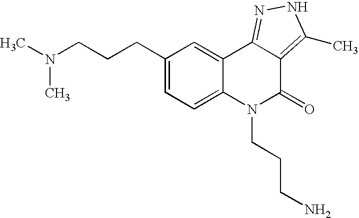

The title compound was prepared from Example 284 using methods similar to Example 294. LCMS: Method FA, Rₜ=0.91 min, [MH⁺=342.31]. ¹H NMR (300 MHz, D₂O) δ 7.76 (s, 1H), 7.41-7.53 (m, 2H), 4.31 (t, 2H), 3.14-3.22 (m, 2H), 3.00-3.09 (m, 2H), 2.88 (s, 6H), 2.80-2.87 (m, 2H), 2.59 (s, 3H), 2.04-2.17 (m, 4H).

### EXAMPLE 297

### Preparation of 5-(3-Amino-propyl)-3-methyl-8-(3-pyrrolidin-1-yl-propyl)-2,5-dihydro-pyrazolo[4,3-c]quinolin-4-one

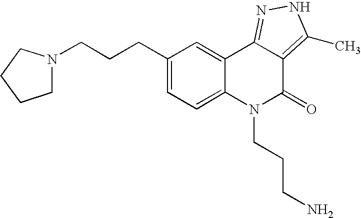

The title compound was prepared from Example 285 using methods similar to Example 294.

To a solution of 5-(3-Amino-propyl)-3-methyl-8-(3-pyrrolidin-1-yl-prop-1-ynyl]-2,5-dihydro-pyrazolo[4,3-c]quinolin-4-one (180 mg, 0.45 mmol) in 6 ML MeOH, 1 ML ethyl acetate, and 2 mL DCM under Argon was added 10% palladium on carbon (20 mg). The mixture was then stirred under 1 atm hydrogen at room temperature for 16 hours. The mixture was filtered through celite, and the solvents were evaporated. The residue was triturated with ether, filtered and washed with ether to give 30.6 mg product as a white solid. LCMS: Method FA, Rₜ=0.69 min, [MH⁺=368.33]. ¹H NMR (300 MHz, D₂O) δ 7.67 (s, 1H), 7.46 (d, 1H), 7.38 (d, 2H), 4.25 (t, 2H), 3.62-3.72 (m, 2H), 3.19-3.27 (m, 2H), 2.98-3.11 (m, 4H), 2.81 (t, 2H), 2.55 (s, 3H), 1.93-2.17 (m, 8H).

### EXAMPLE 298

### Preparation of N-{3-[5-(3-Amino-propyl)-3-methyl-4-oxo-4,5-dihydro-2H-pyrazolo[4,3-c]quinolin-8-yl]-propyl}-acetamide

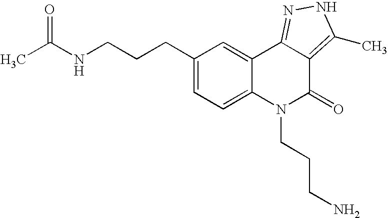

The title compound was prepared from Example 276 using methods similar to Example 294. ¹H NMR 300 MHz (DMSO) δ 8.06 (bs, 1H), 7.99 (bs, 1H), 7.42-7.63 (m, 2H), 4.36 (t, 2H), 3.08-3.15 (m, 2H), 2.85-2.95 (m, 2H), 2.69-2.77 (m, 2H), 2.61 (s, 3H), 1.95-2.05 (m, 2H), 1.86 (s, 3H), 1.75-1.86 (m, 2H).

### EXAMPLE 299

### Preparation of 5-(3-Amino-propyl)-3-methyl-8-(2-pyridin-2-yl-thyl)-2,5-dihydro-pyrazolo[4,3-c]quinolin-4-one

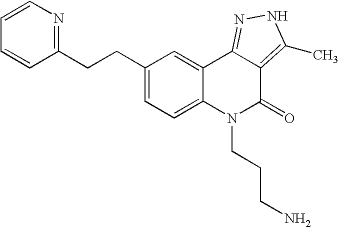

The title compound was prepared from Example 278 using methods similar to Example 294. ¹H NMR 300 MHz (DMSO) δ 8.83 (d, 1H), 8.47 (t, 1H), 8.08 (d, 1H), 7.85-8.00 (m, 3H), 7.50-7.63 (m, 2H), 4.35 (t, 2H), 3.41-3.49 (m, 2H), 3.19-3.27 (m, 2H), 2.85-2.94 (m, 2H), 2.61 (s, 3H), 1.93-2.02 (m, 2H).

### EXAMPLE 300

### Preparation of 8-Ethyl-3-methyl-5-(3-methylamino-propyl)-2,5-dihydro-pyrazolo[4,3-c]quinolin-4-one

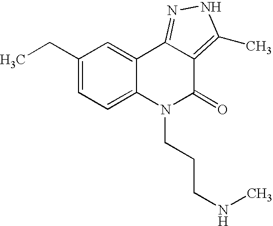

The title compound was prepared from Example 293 using methods similar to Example 294. ¹H NMR 300 MHz (CD₃OD) δ 8.45 (bs, 2H), 8.03 (s, 1H), 7.57 (d, 1H), 7.47 (d, 1H), 4.36 (t, 2H), 2.75 (q, 2H), 2.60 (s, 3H), 2.59 (s, 3H), 2.95-3.05 (m, 2H), 1.95-2.05 (m, 2H), 1.29 (t, 3H). LCMS: Method FA, Rₜ=0.80 min, [MH⁺=271.2].

### EXAMPLE 301

### Preparation of 5-(3-Amino-propyl)-8-(3-hydroxy-propyl)-3-methyl 2,5-dihydro-pyrazolo[4,3-c]quinolin-4-one

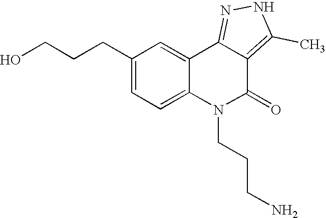

The title compound was prepared from {3-[8-(3-Hydroxy-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 272.

To a solution of {3-[8-(3-Hydroxy-prop-1-ynyl)-3-methyl-4-oxo-2-(tetrahydro-pyran-2-yl)-2,4-dihydro-pyrazolo[4,3-c]quinolin-5-yl]-propyl}-carbamic acid tert-butyl ester (200 mg, 0.404 mmol) in 5mL MeOH, 1 mL ethyl acetate, and 1 mL DCM under Argon was added 10% palladium on carbon (36 mg). The mixture was then stirred under 50 psi hydrogen at room temperature for 16 hours. The mixture was filtered through celite, and the solvents were evaporated. The residue was purified by silica gel chromatography (ISCO, elution with 10-60% ethyl acetate in hexanes) to give 70 mg of {3-[8-(3-Hydroxy-propyl)-3-methyl-4-oxo-2-(tetrahydro-pyran-2-yl)-2,4-dihydro-pyrazolo[4,3-c]quinolin-5-yl]-propyl}-carbamic acid tert-butyl ester (higher eluting spot) and 73 mg of {3-[3-Methyl-4-oxo-8-propyl-2-(tetrahydro-pyran-2-yl)-2,4-dihydro-pyrazolo[4,3-c]quinolin-5-yl]-propyl}-carbamic acid tert-butyl ester (lower eluting spot), both as white solids.

The protected material was dissolved (73 mg, 0.15 mmol) in 4 mL DCM and 1 ml MeOH at room temperature was added 2 ml 4.0 M HCl in dioxane. The solution was stirred at room temperature for 6 hours. Ether was added and the precipitate was then filtered and washed with ether to give 37.6 mg product as a white solid. LCMS: Method FA, Rₜ=0.83 min, [MH⁺=315.20]. ¹H NMR (300 MHz, D₂O) δ 7.43 (s, 1H), 7.35 (d, 1H), 7.21 (d, 1H), 4.10 (t, 2H), 3.64 (t, 2H), 2.97 (t, 2H), 2.68 (t, 2H), 2.46 (s, 3H), 1.99 (m, 2H), 1.86 (m, 2H).

### EXAMPLE 302

### Preparation of 5-(3-Amino-propyl)-3-methyl-8-propyl-2,5-dihydro-pyrazolo[4,3-c]quinolin4-one

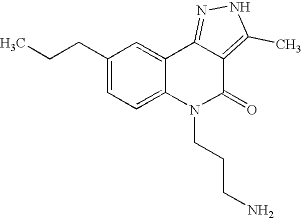

The title compound was prepared from the side product, {3-[3-Methyl-4-oxo-8-propyl-2-(tetrahydro-pyran-2-yl)-2,4-dihydro-pyrazolo[4,3-c]quinolin-5-yl]-propyl}-carbamic acid tert-butyl ester, isolated from Example 301.

The HCl salt of the title compound was prepared as in Example 301: (70 mg, 0.145 mmol) in 4 mL DCM and 1 ml MeOH at room temperature was added 2 ml 4.0 M HCl in dioxane. The solution was stirred at room temperature for 6 hours. Ether was added and the precipitate was then filtered and washed with ether to give 39.2 mg product as a white solid. LCMS: Method FA, Rₜ=1.02 min, [MH⁺=299.17]. ¹H NMR (300 MHz, D₂O) δ 7.34 (m, 1H), 7.29 (d, 1H), 7.15 (d, 1H), 4.06 (t, 2H), 2.96 (t, 2H), 2.54 (t, 2H),-2.44 (s, 3H), 1,97 (m, 2H), 1.58 (m, 2H), 0.91 (t, 3H).

### EXAMPLE 303

### Preparation of 8-Ethynyl-3-methyl-5-piperidin-3-ylmethyl-2,5-dihydro-pyrazolo[4,3-c]quinolin-4-one

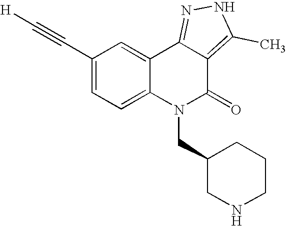

The title compound was prepared by methods similar to those outlined in Example 291 using the appropriate reagents. ¹H NMR 300 MHz (DMSO) δ 8.41 (bs, 1H), 8.26 (d, 1H), 7.58-7.69 (m, 2H), 4.26-4.36 (m, 1H), 4.28 (s, 1H), 4.07-4.18 (m, 1H), 2.98 (t, 2H), 2.60 (s, 3H), 2.56-2.66 (obscured by solvent m, 1H), 2.02-2.12 (bm, 1H), 1.74 (t, 2H), 1.25-1.49 (m, 3H).

### EXAMPLE 304

### Preparation of 8-Ethynyl-3-ethyl-5-(3-methylamino-propyl)-2,5-dihydro-pyrazolo[4,3-c]quinolin-4-one

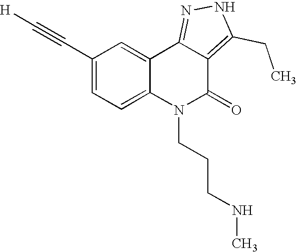

The title compound was prepared from the appropriate reagents as outlined in Example 291. ¹H NMR 300 MHz (MeOD) δ 8.40 (d, 1H), 7.80 (dd, 1H), 7.71 (d, 1H), 4.59 (t, 2H), 3.71 (s, 1H), 3.23 (q, 2H), 3.12 (t, 2H), 2.81 (bs, 1H), 2.26 (t, 1H), 1.49 (t, 3H).

### EXAMPLE 305

### Preparation of 5-(3-Amino-propyl)-3-(2-methoxy-ethyl)-8-(3-pyrrolidin-1-yl-prop-1-ynyl)-2,5-dihydro-pyrazolo[4,3-c]quinolin-4-one

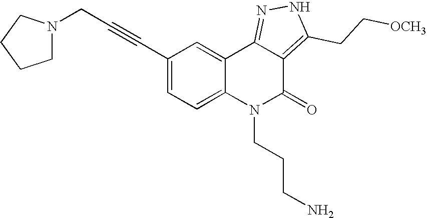

The title compound was prepared in a manner similar to Example 272 using 1-Prop-2-ynyl-pyrrolidine as the coupling partner, or by methods outlined in Example 285.

### Preparation of 1-Prop-2-ynyl-pyrrolidine

To a solution of pyrrolidine (11.5 mL, 139 mmol) in Et₂O at 0° C. was added 3-Bromo-propyne (10.3 g, 69.3 mmol) slowly via syringe and the mixture was refluxed for 12 h. The organic layer was decanted, the remaining oil was extracted with Et₂O and the combined organics were dried over K₂CO₃, filtered and concentrated in vacuo. Vacuum distillation (74-77° C., 85 mmHg) afforded 4.59 g (61%) of the title compound as a clear oil. ¹H NMR (300 MHz, CDCl₃) δ 3.41 (d, J=2.4 Hz, 2H), 2.56-2.74 (m, 4H), 2.19 (t, J=2.4 Hz, 1H), 1.77-1.92 (m, 4H) ppm; LC/MS: AA standard Rₜ=0.64 min, EI⁺ 109.96.

Title compound: 5-(3-Amino-propyl)-3-(2-methoxy-ethyl)-8-(3-pyrrolidin-1-yl-prop-1-ynyl)-2,5-dihydro-pyrazolo[4,3-c]quinolin-4-one: ¹H NMR (300 MHz, DMSO-d₆) δ 11.6 (s, 1H), 8.40 (s, 1H), 8.02 (br s, 3H), 7.65-7.76 (m, 2H), 4.41 (d, J=4.3 Hz, 2H), 4.35 (t, J=6.1 Hz, 2H), 3.72 (t, J=6.8 Hz, 2H), 3.52-3.64 (m, 2H), 3.25 (s, 3H), 3.11-3.23 (m, 4H), 2.82-2.96 (m, 2H), 1.86-2.15 (m, 6H) ppm; LC/MS: AA standard Rₜ=0.97 min, EI⁺ 408.39.

### EXAMPLE 306

### Preparation of 4-[5-(3-Amino-propyl)-3-methyl-4-oxo-4,5-dihydro-2H-pyrazolo[4,3-c]quinolin-8-yl]-benzonitrile

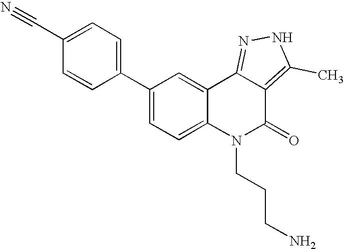

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 56.

### Step 1: Preparation of {3-[8-(4-Cyano-phenyl)-3-methyl-4-oxo-2-(tetrahydro-pyran-2-yl)-2,4-dihydro-pyrazolo[4,3-c]quinolin-5-yl]-propyl}-carbamic acid tert-butyl ester

To a suspension of {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester (200 mg, 0.385 mmol) in toluene (4 mL) was added EtOH (1 mL), and sodium carbonate (0.700 mL, 10% aq solution). The mixture was sparged with Ar (15 min) and palladium tetrakis triphenylphosphine (22 mg, 0.0192 mmol) and 4-cyanobenzeneboronic acid (62 mg, 0.4238 mmol) were added. The reaction was heated (80° C.) stirred 4 h, cooled, and poured into sodium bicarbonate (saturated aq solution). The mixture was diluted with EtOAc, the layers separated. The organic layer was washed (water, brine), dried (MgSO₄), filtered, and concentrated in vacuo. Flash chromatography (gradient elution, 0-75% EtOAc/hexanes) provided 180 mg of the title compound (white solid). LCMS: Method FA, Rₜ=2.24 min, [MH⁺=542.2].

### Step 2: Preparation of 4-[5-(3-Amino-propyl)-3-methyl-4-oxo-4,5-dihydro-2H-pyrazolo[4,3-c]quinolin-8-yl]-benzonitrile

To a solution of {3-[8-(4-Cyano-phenyl)-3-methyl-4-oxo-2-(tetrahydro-pyran-2-yl)-2,4-dihydro-pyrazolo[4,3-c]quinolin-5-yl]-propyl}-carbamic acid tert-butyl ester (180 mg, 0.333 mmol) in MeOH (4 mL) was added concentrated aq HCl (0.1 mL). The reaction was sealed and stirred 12 h then concentrated in vacuo to afford 149 mg of the title compound (white solid). LCMS: Method FA, Rₜ=1.05 min, [MH⁺=358.2]. LCMS: Method FA, Rₜ=1.05 min, [MH⁺=358.2]; ¹HNMR (300 MHz, CD₃OD) δ 8.54 (d, 1 H), 7.99 (dd, 1 H), 7.94 (d, 2 H), 7.87 (d, 2 H), 7.75 (d, 1 H), 4.53 (dd, 2 H), 3.04 (dd, 2 H), 2.71 (s, 3 H), 2.24-2.15 (dd, 2 H).

### EXAMPLE 307

### Preparation of 5-(3-Amino-propyl)-8-(4-dimethylamino-phenyl)-3-methyl-2,5-dihydro-pyrazolo [4,3-c]quinolin-4-one

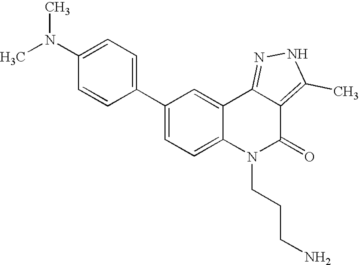

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester as in Example 306. LCMS: Method FA, Rₜ=1.02 min, [MH⁺=382.1]; ¹HNMR (300 MHz, CD₃OD) δ 8.53 (s, 1 H), 8.02-7.95 (m, 3 H), 7.82 (d, 2 H), 7.76 (d, 1 H), 4.53 (dd, 2 H), 3.38 (s, 6 H), 3.04 (dd, 2 H), 2.72 (s, 3 H), 2.25-2.15 (m, 2 H).

### EXAMPLE 308

### Preparation of 5-(3-Amino-propyl)-3-methyl-8-pyridin-3-yl-2,5-dihydro-pyrazolo[4,3-c]quinolin-4-one

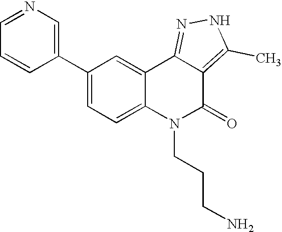

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester as in Example 306. LCMS: Method FA, Rₜ=0.72 min, [MH⁺=334.2]; ¹HNMR (300 MHz, CD₃OD) δ 9.30 (s, 1 H), 9.05 (d, 1 H), 8.88 (d, 1 H), 8.68 (d, 1 H), 8.24 (dd, 1 H), 8.11 (dd, 1 H), 7.85 (d, 1 H), 4.54 (dd, 2 H), 3.06 (dd, 2 H), 2.74 (s, 3 H), 2.26-2.15 (m, 2 H).

### EXAMPLE 309

### Preparation of 5-(3-Amino-propyl)-8-furan-3-yl-3-methyl-2,5-dihydro-pyrazolo[4,3-c]quinolin-4-one

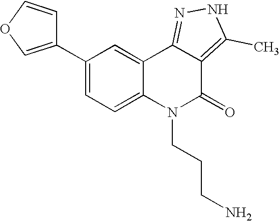

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester as in Example 306. LCMS: Method FA, Rₜ=0.97 min, [MH⁺=323.2]; ¹HNMR (300 MHz, CD₃OD) δ 8.32 (s, 1 H), 8.00 (s, 1 H), 7.84 (dd, 1 H), 7.65-7.61 (m, 2 H), 6.90-6.89 (m, 1 H), 4.48 (dd, 2 H), 3.02 (dd, 2 H), 2.70 (s, 3 H), 2.22-2.13 (m, 2 H).

### EXAMPLE 310

### Preparation of 5-(3-Amino-propyl)-3-methyl-8-pyrrolidin-1-yl-2,5-dihydro-pyrazolo[4,3-c]quinolin-4-one

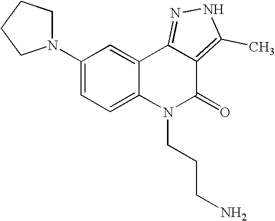

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester, Example 56 by methods similar to Example 120.

### Step 1: Preparation of {3-[3-Methyl-4-oxo-8-pyrrolidin-1-yl-2-(tetrahydro-pyran-2-yl)-2,4-dihydro-pyrazolo[4,3-c]quinolin-5-yl]-propyl}-carbamic acid tert-butyl ester

To a suspension of {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester (100 mg, 0.193 mmol) in toluene (5 mL) was added pyrrolidine (0.027 mL,0.327 mmol), Pd₂(dba)₃ (18 mg, 0.0193 mmol), (2′-Dicyclohexylphosphanyl-biphenyl-2-yl)-dimethyl-amine (10 mg, 0.0231 mmol), and sodium tert-butoxide (95 mg, 0.965 mmol). The reaction was sparged with Ar for 15 min, stirred under Ar, and heated (80° C.). After 1 h, the solution was cooled, and poured into sodium bicarbonate (saturated aq solution). The mixture was diluted with EtOAc, the layers separated. The organic layer was washed (water, brine), dried (MgSO₄), filtered, and concentrated in vacuo. Flash chromatography (gradient elution, 0-50% EtOAc/hexanes) provided 80 mg of the title compound (white solid). LCMS: Method FA, Rₜ=2.51 min, [MH⁺=510.6].

### Step 2: Preparation of5-(3-Amino-propyl)-3-methyl-8-pyrrolidin-1-yl-2,5-dihydro-pyrazolo[4,3-c]quinolin-4-one

To a solution of {3-[3-Methyl-4-oxo-8-pyrrolidin-1-yl-2-(tetrahydro-pyran-2-yl)-2,4-dihydro-pyrazolo[4,3-c]quinolin-5-yl]-propyl}-carbamic acid tert-butyl ester (80 mg, 0.333 mmol) in MeOH (4 mL) was added concentrated aq HCl (0.1 mL). The reaction was sealed and stirred 12 h then concentrated in vacuo to afford 89 mg of the title compound (white solid). LCMS: Method FA, Rₜ=0.97 min, [MH⁺=326.2].). LCMS: Method FA, Rₜ0.97 min, [MH⁺=326.2]; ¹HNMR (300 MHz, CD₃OD) δ 8.31 (s, 1 H), 7.81-7.76 (m, 2 H), 4.49 (dd, 2 H), 3.88-3.80 (m, 4 H), 3.04 (dd, 2 H), 2.72 (s, 3 H), 2.38-2.32 (m, 4 H), 2.21-2.11 (m, 2 H).

### EXAMPLE 311

### Preparation of 5-(3-Amino-propyl)-8-chloro-3-ethyl-2,5-dihydro-pyrazolo[4,3-c]quinolin-4-one hydrochloride

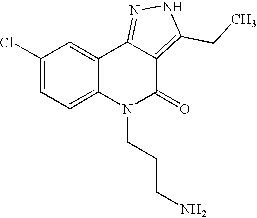

### Step 1: Preparation N-Methoxy-N-methyl-malonamic acid

To a solution of sodium hydroxide (11.1 g, 278 mmol) in water (35 mL) at 0° C. was added O,N-dimethyl-hydroxylamine hydrochloride (27.1 g, 278 mmol), then 2,2-dimethyl-[1,3]dioxane-4,6-dione (10.0 g, 69 mmol). The reaction was allowed to warm to room temperature and stirred for 18 h. The reaction was cooled again to 0° C. and concentrated hydrochloric acid (17 mL) was added dropwise. The mixture was extracted into chloroform (5×) and the organic phases were dried (Na₂SO₄) and evaporated. The residue was purified by filtration through a pad of silica, eluting with 20% ethyl acetate/hexane then 10% methanol/dichloromethane to give the desired product as an oil (5.88 g, 60%). LCMS: ES⁺148.02 (M+1), ES⁻146.85 (M−1).

### Step 2: Preparation of 5-Chloro-2-[2-(methoxy-methyl-carbamoyl)-acetylamino]-benzoic acid methyl ester

To a solution of N-Methoxy-N-methyl-malonic acid (13.6 g, 93 mmol) and 2-Amino-5-chloro-benzoic acid methyl ester (17.2 g, 93 mmol) in dichloromethane (300 mL), was added triethylamine (25.8 mL, 185 mmol) then bis(2-oxo-3-oxazolidinyl)phosphinic chloride (25.9 g, 102 mmol). The reaction mixture was stirred for 2 h then acidified by the addition of IN HCl solution. The organic phase was separated, washed with 1N HCl, water, then brine, dried (Na₂SO₄) and evaporated. The residue was purified by filtration through a pad of silica, eluting with 20%, 50% then 70% ethyl acetate/hexane to yield the desired compound as a white solid (19.5 g, 67%). LCMS: ES⁺315.10 (M+1), ES⁻313.04 (M−1).

### Step 3: Preparation of 6-Chloro-4-hydroxy-2-oxo-1,2-dihydro-quinoline-3-carboxylic acid methoxy-methyl-amide

To a solution of 5-Chloro-2-[2-(methoxy-methyl-carbamoyl)-acetylamino]-benzoic acid methyl ester (18.1 g, 58 mmol), in methanol (300 mL), was added sodium methoxide solution (25% wt in methanol, 26.3 mL, 115 mmol). The reaction mixture was heated under reflux for 30 min, then cooled to room temperature and 1N HCl solution (115 mL, 115 mmol) was added. The mixture was cooled to 0° C. and the prepitate was filtered off, washed with water then hexane and dried under vacuum to yield the desired product as a white solid (14.1 g, 87%). LCMS: ES⁺283.10 (M+1), ES⁻281.04 (M−1).

### Step 4: Preparation of 6-Chloro-4-hydroxy-3-propionyl-1H-quinolin-2-one

To a solution of 6-Chloro-4-hydroxy-2-oxo-1,2-dihydro-quinoline-3-carboxylic acid methoxy-methyl-amide (500 mg, 1.8 mmol), in THF (18 mL) and HMPA (1.8 mL) at room temperature, was added ethyl magnesium chloride (2.0 M in Et₂O, 4.43 mL, 8.9 mmol) dropwise. The reaction mixture was stirred for 1 h, then 1N HCl solution was added and the precipitate was filtered off. The solid was washed with water then hexane and dried under vacuum to yield the desired product as a white solid (352 mg, 79%). LCMS: ES⁺252.07 (M+1), ES⁻250.04 (M−1).

### Step 5: Preparation of 8-Chloro-3-ethyl-2,5-dihydro-pyrazolo[4,3-c]quinolin4-one

This compound was prepared from 6-Chloro-4-hydroxy-3-propionyl-1H-quinolin-2-one using a similar procedure to that described in Example 202, Step 4. LCMS: ES⁺248.09 (M+1), ES⁻246.05 (M−1). ¹H NMR (300 MHz, d6 DMSO) δ 13.69 (1 H, Br s), 8.02 (1 H, Br s), 7.46-7.30 (2 H, m), 2.94 (2H, unresolved q), 1.23 (3 H, t).

### Step 6: Preparation of 8-Chloro-3-ethyl-2-(tetrahydro-pyran-2-yl)-2,5-dihydro-pyrazolo[4,3-c]quinolin-4-one

This compound was prepared from compound 8-Chloro-3-ethyl-2,5-dihydro-pyrazolo[4,3-c]quinolin-4-one using a similar procedure to that described in Example 202, Step 5. LCMS: ES⁺332.16 (M+1).

### Step 7: Preparation of {3-[8-Chloro-3-ethyl-4-oxo-2-(tetrahydro-pyran-2-yl)-2,4-dihydro-pyrazolo[4,3-c]quinolin-5-yl]-propyl}-carbamic acid tert-butyl ester

This compound was prepared from the product of Step 6 using alkylation conditions described in Example 202, Step 6. LCMS: ES⁺489.21 (M+1).

### Step 8: Preparation of 5-(3-Amino-propyl)-8-chloro-3-ethyl-2,5-dihydro-pyrazolo[4,3-c]quinolin-4-one

The title compound was prepared from the Step 7 intermediate using a similar procedure to that described in Example 202, Step 7. LCMS: FA, Rₜ=0.98 min ES⁺305.13 (M+1), ES⁻303.09 (M−1). ¹H NMR (300 MHz, MeOD) δ 8.17 (1 H, dd), 7.63-7.76 (2 H, m), 4.47 (2H, t), 3.14 (2 H, q), 3.02 (2 H, t,), 2.20-2.11 (2 H, m), 1.38 (2H, t).

### EXAMPLE 312

### Preparation of 5-(3-Amino-propyl)-8-chloro-3-(3-hydroxy-propyl)-2,5-dihydro-pyrazolo[4,3-c]quinolin-4-one

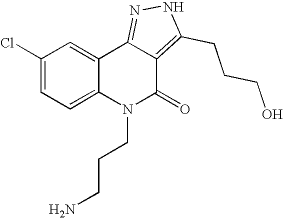

The title compound was prepared from the intermediate from Step 3 in Example 311 using a procedure similar to that outlined in Example 311.

### Step 1: Preparation of 3-(4-Benzyloxy-butyryl)-6-chloro-4-hydroxy-1H-quinolin-2-one

This compound was prepared from 6-Chloro-4-hydroxy-2-oxo-1,2-dihydro-quinoline-3-carboxylic acid methoxy-methyl-amide using (3-benzyloxypropyl)-magnesium bromide, and a procedure similar to that described in Example 311, Step 4. LCMS: ES⁺372.21 (M+1), ES⁻370.17 (M−1).

### Step 2: 3-(3-Benzyloxy-propyl)-8-chloro-2,5-dihydro-pyrazolo[4,3-c]quinolin-4-one

This compound was prepared from 3-(4-Benzyloxy-butyryl)-6-chloro-4-hydroxy-1H-quinolin-2-one using a similar procedure to that described in Example 311, Step 5. LCMS: ES⁺368.20 (M+1). ES⁻366.14.17 (M−1).

### Step 3: Preparation of 3-(3-Benzyloxy-propyl)-8-chloro-2-(tetrahydro-pyran-2-yl)-2,5-dihydro-pyrazolo[4,3-c]quinolin-4-one

This compound was prepared from 3-(3-Benzyloxy-propyl)-8-chloro-2,5-dihydro pyrazolo[4,3-c]quinolin-4-one using a similar procedure to that described in Example 311, Step 6. LCMS: ES⁺452.20 (M+1).

### Step 4: {3-[3-(3-Benzyloxy-propyl)-8-chloro-4-oxo-2-(tetrahydro-pyran-2-yl)-2,4-dihydro-pyrazolo[4,3-c]quinolin-5-yl]-propyl}-carbamic acid tert-butyl ester

This compound was prepared from 3-(3-Benzyloxy-propyl)-8-chloro-2-(tetrahydropyran-2-yl)-2,5-dihydro-pyrazolo[4,3-c]quinolin-4-one using using a similar procedure to that described in Example 311, Step 7. LCMS: ES⁺609.15 (M+1).

### Step 5: Preparation of {3-[8-Chloro-3-(3-hydroxy-propyl)-4-oxo-2-(tetrahydro-pyran-2-yl)-2,4-dihydro-pyrazolo[4,3-c]quinolin-5-yl]-propyl}-carbamic acid tert-butyl ester

To a solution of {3-[3-(3-Benzyloxy-propyl)-8-chloro-4-oxo-2-(tetrahydro-pyran-2-yl)-2,4-dihydro-pyrazolo[4,3-c]quinolin-5-yl]-propyl}-carbamic acid tert-butyl ester (393 mg, 0.6 mmol), in ethanol (7 mL), was added 10% palladium on carbon (60 mg, 15% w/w). The mixture was hydrogenated at atmospheric pressure for 50 min, and then filtered through celite with further ethanol. The filtrate was evaporated then purified by chromatography on silica, eluting with 50% to 100% ethyl acetate/hexane to give the desired product (263 mg, 78%). LCMS: ES⁺519.17 (M+1).

### Step 6: Preparation of 5-(3-Amino-propyl)-8-chloro-3-(3-hydroxy-propyl)-2,5-dihydro-pyrazolo[4,3-c]quinolin-4-one

The title compound was prepared from {3-[8-Chloro-3-(3-hydroxy-propyl)-4-oxo-2 (tetrahydro-pyran-2-yl)-2,4-dihydro-pyrazolo[4,3-c]quinolin-5-yl]-propyl}-carbamic acid tert-butyl ester using a similar procedure to that described in Example 311, Step 8. LCMS: FA, Rₜ=0.89 min, ES⁺335.12 (M+1). ¹H NMR (300 MHz, MeOD) δ 8.17 (1 H, d), 7.62-7.79 (2 H, m), 4.47 (2 H, t), 3.63 (2 H, t), 3.18 (2H, t), 3.01 (2H, t), 2.20-2.10 (2H, m), 2.07-1.79 (2H, m).

### EXAMPLE 313

### Preparation of 5-(3-Amino-propyl)-8-chloro-3-(4-hydroxy-butyl)-2,5-dihydro-pyrazolo[4,3-c]quinolin-4-one

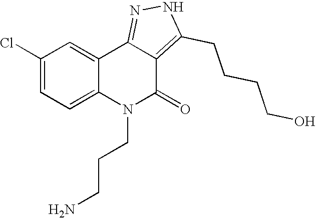

The title compound was prepared by methods outlined in Example 312. LCMS: FA, rt=0.91 min, ES⁺349.17 (M+1), ES⁻347.13 (M−1). ¹H NMR (300 MHz, MeOD) δ 8.13-8.12 (1 H, m), 7.57-7.54 (2 H, m), 4.42 (2 H, t), 3.55 (2 H, t), 3.09 (2 H, t), 2.96 (2 H, t), 2.14-2.05 (2 H, m), 1.88-1.77 (2 H, m), 1.51-1.51 (2H, m).

### EXAMPLE 314

### Preparation of 5-(3-Amino-propyl)-8-chloro-3-(4-methoxy-butyl)-2,5-dihydro-pyrazolo[4,3-c]quinolin4-one

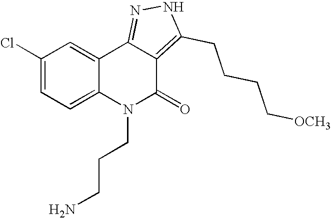

The title compound was prepared from {3-[8-Chloro-3-(4-methoxy-butyl)-4-oxo-2-(tetrahydro-pyran-2-yl)-2,4-dihydro-pyrazolo[4,3-c]quinolin-5-yl]-propyl-carbamic acid tert-butyl ester, the protected intermediate from Example 313.

To a solution of the Boc/THP protected intermediate (90 mg, 0.17 mmol), in THF at 0° C., was added sodium hydride (8 mg, 60% dispersion in oil, 0.20 mmol). The reaction was stirred at 0° C. for 30 min, and then warmed to room temperature. Iodomethane (12.6 uL, 0.20 mmol), was added and the reaction heated at 65° C. for 45 min. Saturated ammonium chloride solution was added and the mixture was extracted with ethyl acetate (3×). The combined organic phases were dried (Na₂SO₄) and evaporated. The residue was purified by chromatography on silica eluting with 0% to 50% ethyl acetate/hexane to give 74 mg of a 1:1 mixture of mono- and di-methylated products. The mixture was dissolved in methanol (2 mL) and a solution of HCl in diethyl ether (2.0M, 2 mL) was added. The mixture was stirred for 3 h, and then the solvents were evaporated. The residue was purified by HPLC to yield the desired products 21 (25 mg, 38%), and 22 (18 mg, 26%). LCMS: FA, Rₜ=0.98 min ES⁺363.19(M+1), ES⁻361.13(M−1). ¹H NMR (300 MHz, MeOD) δ 8.19-8.18(1H, m), 6.63-7.62 (2 H, m), 4.48 (2 H, t), 3.45 (2H, t), 3.32 (3 H s, overlaid with MeOD) 3.15 (2 H, t), 3.02 (2 H, t), 2.21-2.11 (2 H, m,), 1.93-1.83 (2 H, m), 1.70-1.61 (2 H, m).

### EXAMPLE 315

### Preparation of 8-Chloro-3-(4-methoxy-butyl)-5-(3-methyl-amino-propyl)-2,5-dihydro-pyrazolo[4,3-c]quinolin-4-one

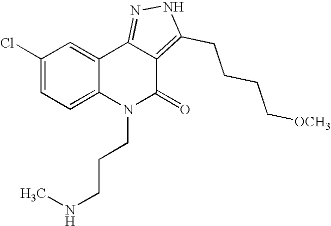

The title compound was isolated as a side product from Example 314. LCMS: FA, Rₜ=1.01 min ES⁺377.19 (M+1), ES⁻375.15 (M−1). ¹H NMR (300 MHz, MeOD) δ 8.12-8.11 (1 H, m), 7.57-7.55 (2 H, m), 4.41 (2 H, t), 3.37 (2 H, t), 3.25 (3H, s, overlaid with MeOD), 3.08 (2 H, t), 3.01 (2 H, t), 2.69 (3 H, s,) 2.16-2.07 (2 H, m), 1.86-1.76 (2 H, m), 1.63-1.53 (2 H, m).

### EXAMPLE 316

### Preparation of 5-(3-Amino-propyl)-8-chloro-3-(2-hydroxy-ethyl)-2,5-dihydro-pyrazolo[4,3-c]quinolin-4-one

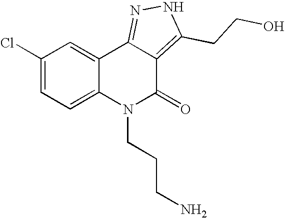

### Step 1: Preparation of 5-Benzyloxy-3-oxo-pentanoic acid methyl ester

To a suspension of sodium hydride (5.5 g of 60% suspension in mineral oil, 140 mmol) in THF (100 mL) at 0° C. under argon was added a 20 mL solution of methylacetoacetate (15 g, 130 mmol) in THF dropwise over 30 min. After stirring for 30 min., the reaction mixture was cooled to −25° C. Butyllithium (57 mL of 2.5M solution in hexanes, 140 mmol) was added dropwise to the reaction mixture, which was then stirred for 45 min. A solution of Chloromethoxymethyl-benzene (22 g, 140 mmol) in 10 mL THF was then added slowly to the reaction mixture, which was stirred for 1 h. The reaction was then diluted up with 100 mL cold 1N HCl (aq) and 100 mL CH₂Cl₂. The aqueous phase was washed two times with CH₂Cl₂. The organic extracts were combined, dried with MgSO₄, filtered, and evaporated to yield a dark brown residue which was purified by flash chromatography (silica gel column) with ethyl acetate and hexanes (25:75 v/v) to provide the title compound (16.9 g, 56%). ¹H NMR (300 MHz, CDCl₃) δ 2.81 (2H, t, J=6.2 Hz), 3.49 (2H, s), 3.71 (3H, s), 3.74 (2H, t, J=6.2 Hz), 4.50 (2H, s), 7.24-7.38 (5H, m).

### Step 2: Preparation of 2-(5-Benzyloxy-3-oxo-pentanoylamino)-5-chloro-benzoic acid methyl ester

This compound was made according to procedures outlined in Example 202, Step 2 using 2-Amino-5-chloro-benzoic acid methyl ester (13.2 g, 71 mmol) and 5-Benzyloxy-3-oxo-pentanoic acid methyl ester (16.9 g, 72 mmol). The product was chromatographed with a 330 g silica column (EtOAc/hexanes 15:85) to afford the desired product (9.9 g, 40%). ¹H NMR (300 MHz, CDCl₃) δ 2.89 (2H, t, J=6.1 Hz), 3.64 (2H, s), 3.79 (2H, d, J=6.1 Hz), 3.94 (3H, s), 4.51 (2H, s), 7.25-7.38 (5H, m), 7.48 (1H, dd, J=2.6, 9.0 Hz), 8.0 (1H, d, J=2.6 Hz), 8.64 (1H, d, J=9.0 Hz).

### Step 3: Preparation of 3-(3-Benzyloxy-propionyl)-6-chloro-4-hydroxy-1H-quinolin-2-one

This compound was made according to procedures outlined in Example 202, Step 3 starting with 2-(5-Benzyloxy-3-oxo-pentanoylamino)-5-chloro-benzoic acid methyl ester (6.0 g, 17 mmol) to form the desired product (4.4 g, 72%). ¹H NMR (300 MHz, DMSO) δ 3.49 (2H, t, J=6.3 Hz), 3.77 (2H, t, J=6.3 Hz), 4.47 (2H, s), 7.12-7.40 (5H, m), 7.32 (1H, d, J=8.8 Hz), 7.69 (2H, dd, J=2.3,8.8 Hz), 7.88 (1H, d, J=2.3 Hz). LCMS: Method FA, Rₜ=1.80 min, [MH⁺=358.08].

### Step 4: Preparation of 3-(2-Benzyloxy-ethyl)-8-chloro-2,5-dihydro-pyrazolo[4,3-c]quinolin-4-one

This compound was made according to procedures outlined in Example 202, Step 4 starting with 3-(3-Benzyloxy-propionyl)-6-chloro-4-hydroxy-1H-quinolin-2-one (4.4 g, 12 mmol) to form the desired product (3.4 g, 78%). ¹H NMR (300 MHz, DMSO) δ 3.20 (2H, t, J=7.0 Hz), 3.77 (2H, t, J=7.0 Hz), 4.43 (2H, s), 7.15-7.27 (5H, m), 7.32 (1H, d, J=8.9 Hz), 7.43 (2H, dd, J=2.3, 8.9 Hz), 8.04 (1H, d, J=2.3 Hz). LCMS: Method FA, Rₜ=1.76 min, [MH⁺=354.20].

### Step 5: Preparation of 3-(2-Benzyloxy-ethyl)-8-chloro-2-(tetrahydro-pyran-2-yl)-2,5-dihydro-pyrazolo[4,3-c]quinolin-4-one

This compound was made according to procedures outlined in Example 202, Step 5 starting with 3-(2-Benzyloxy-ethyl)-8-chloro-2,5-dihydro-pyrazolo[4,3-c]quinolin-4-one (3.4 g, 10 mmol) to form the desired product (3.9 g, 88%). This compound was carried on crude to the next step without any purification. LCMS: Method FA, Rₜ=2.18 min, [MH⁺=438.07].

### Step 6: Preparation of {3-[3-(2-Benzyloxy-ethyl)-8-chloro-4-oxo-2-(tetrahydro-pyran-2-yl)-2,4-dihydro-pyrazolo[4,3-c]quinolin-5-yl]-propyl}-carbamic acid tert-butyl ester

This compound was made according to procedures outlined in Example 202, Step 6 starting with 3-(2-Benzyloxy-ethyl)-8-chloro-2-(tetrahydro-pyran-2-yl)-2,5-di-hydro-pyrazolo-[4,3-c]quinolin-4-one (1.0 g, 2.3 mmol) to form the desired product (290 mg, 22%). This compound was carried on crude to the next step without any purification. LCMS: Method FA, Rₜ=2.55 min, [MH⁺=595.16].

### Step 7: Preparation of {3-[8-Chloro-3-(2-hydroxy-ethyl)-4-oxo-2-(tetrahydro-pyran-2-yl)-2,4-dihydro-pyrazolo[4,3-c]quinolin-5-yl]-propyl}-carbamic acid tert-butyl ester

A solution of {3-[3-(2-Benzyloxy-ethyl)-8-chloro-4-oxo-2-(tetrahydro-pyran-2-yl)-2,4-dihydro-pyrazolo[4,3-c]quinolin-5-yl]-propyl}-carbamic acid tert-butyl ester (290 mg, 0.50 mmol) in 6 mL EtOH was purged with nitrogen. A catalytic amount of Pd/C (44 mg of Pd/C 15% by weight) was then added to the reaction mixture. The flask was flushed with hydrogen at atmospheric pressure with a balloon. The reaction was stirred under hydrogen at atmospheric pressure for 2 h at RT. The reaction mixture was then filtered over celite. The filtrate was evaporated to afford a mixture of the desired product (80%) and the starting material (20%) (220 mg, 88% total yield). LCMS: Method FA, Rₜ=2.04 min, [MH⁺=505.14].

### Step 8: Preparation of 5-(3-Amino-propyl)-8-chloro-3-(2-hydroxy-ethyl)-2,5-dihydro-pyrazolo[4,3-c]quinolin-4-one

To a solution of {3-[8-Chloro-3-(2-hydroxy-ethyl)-4-oxo-2-(tetrahydro-pyran-2-yl)-2,4-dihydro-pyrazolo[4,3-c]quinolin-5-yl]-propyl}-carbamic acid tert-butyl ester (87 mg, 0.18 mmol) in 2 mL CH₂Cl₂ was added 1.5 mL of 1.0N HCl solution in ether. The reaction was stirred for 18 h at rt. The reaction mixture was evaporated and purified by HPLC to afford the desired product.(29 mg, 50%). LCMS: Method FA, Rₜ=0.86 min, [MH⁺=321.11]. ¹H NMR 300 MHz (MeOD) δ 8.15 (t, 1H), 7.57 (d, 2H), 4.44 (t, 2H), 3.95 (t, 2H), 3.25-3.35 (m, 2H), 2.95 (t, 2H), 2.11 (t, 2H).

### EXAMPLE 317

### Preparation of 5-(3-Amino-propyl)-3-(2-hydroxy-ethyl)-2,5-dihydro-pyrazolo[4,3-c]quinolin-4-one

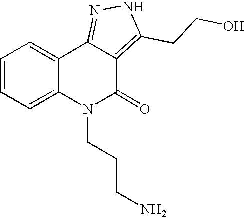

The title compound was prepared from the appropriate reagents by methods outlined in Example 316. LCMS: Method FA, Rₜ=1.41 min, [MH⁺=287.2]; ¹HNMR (300 MHz, CD₃OD) δ 8.17 (d, 1 H), 7.70-7.67 (m, 2 H), 7.43-7.38 (m, 1 H), 4.50 (dd, 2 H), 3.98 (dd, 2 H), 3.34 (dd, 2 H), 3.02 (dd, 2 H), 2.22-2.12 (m, 2 H).

### EXAMPLE 318

### Preparation of 5-(3-Amino-propyl)-8-chloro-3-(2-dimethyl-amino-ethyl)-2,5-dihydro-pyrazolo[4,3-c]quinolin-4-one

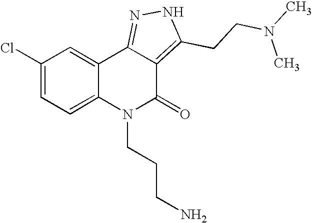

The title compound was prepared from 5-(3-Amino-propyl)-8-chloro-3-(2-hydroxy ethyl)-2,5-dihydro pyrazolo[4,3-c]quinolin-4-one the intermediate from Example 316, Step 7.

### Step 1: Preparation of {3-[8-Chloro-4-oxo-3-(2-oxo-ethyl)-2-(tetrahydro-pyran-2-yl)-2,4-dihydro-pyrazolo[4,3-c]quinolin-5-yl]-propyl}-carbamic acid tert-butyl ester

To a solution of 5-(3-Amino-propyl)-8-chloro-3-(2-hydroxy-ethyl)-2,5-dihydro-pyrazolo[4,3-c]quinolin-4-one (177 mg, 0.35 mmol) in 4 mL CH₂Cl₂ was added a suspension of Dess-Martin Periodinane (223 mg, 0.53 mmol) in 2 mL CH₂Cl₂. The reaction was stirred for 1 h at RT. The reaction mixture was then diluted with NaHCO₃ (aq) and extracted with CH₂Cl₂. The organic layer was dried with MgSO₄, filtered, and evaporated to form the desired product (152 mg, 87%) which was carried on crude to the next step without purification.

### Step 2: Preparation of {3-[8-Chloro-3-(2-dimethylamino-ethyl)-4-oxo-2-(tetrahydro-pyran-2-yl)-2,4-dihydro-pyrazolo[4,3-c]quinolin-5-yl]-propyl}-carbamic acid tert-butyl ester

To a solution of {3-[8-Chloro-4-oxo-3-(2-oxo-ethyl)-2-(tetrahydro-pyran-2-yl)-2,4-dihydro-pyrazolo[4,3-c]quinolin-5-yl]-propyl}-carbamic acid tert-butyl ester in 3 mL CH₂Cl₂ was added dimethylamine (150 uL of 2M solution in THF, 0.3 mmol), sodium triacetoxyborohydride (128 mg, 0.6 mmol), and a catalytic amount of acetic acid (2 drops). The reaction was stirred at RT for 48 h. The reaction mixture was then diluted up with water and extracted with CH₂Cl₂. The organic layer was dried with MgSO₄, filtered, and evaporated to form the desired product which was carried on crude to the next step without any purification. LCMS: Method FA, Rₜ=1.54 min, [MH+=532.39].

### Step 3: Preparation of 5-(3-Amino-propyl)-8-chloro-3-(2-dimethylamino-ethyl)-2,5-dihydro-pyrazolo[4,3-c]quinolin-4-one

To a solution of {3-[8-Chloro-3-(2-dimethylamino-ethyl)-4-oxo-2-(tetrahydro-pyran-2-yl)-2,4-dihydro-pyrazolo[4,3-c]quinolin-5-yl]-propyl}-carbamic acid tert-butyl ester in 2 mL CH₂Cl₂ was added 2 mL of 1.0N HCl solution in ether. The reaction was stirred for 18 h at RT. The reaction mixture was evaporated to afford a while solid (30 mg, 29% over 2 steps). LCMS: Method FA, Rₜ=0.84 min, [MH+=348.14]. ¹H NMR 300 MHz (DMSO-d₆) δ 8.28-8.31 (m, 1H), 7.80-7.90 (m, 1H), 7.60-7.75 (m, 1H), 4.25-4.38 (m, 2H), 3.28-3.50 (m, 4H), 2.70-2.90 (bs, 2H), 2.30-2.50 (m, 2H), 1.80-1.95 (m, 2H).

### EXAMPLE 319

### Preparation of 5-(3-Amino-propyl)-8-chloro-3-(2-piperidin-1-yl-ethyl)-2,5-dihydro-pyrazolo[4,3-c]quinolin-4-one

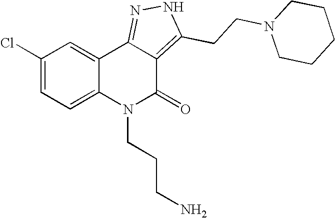

The title compound was prepared using the appropriate reagents in a manner similar to Example 318. LCMS: Method FA, Rₜ=0.82 min, [MH⁺=388.2]; ¹HNMR (300 MHz, CD₃OD) δ 8.15 (s, 1 H), 7.73-7.63 (m, 2 H), 4.50 (dd, 2 H), 3.74-3.65 (m 2 H), 3.61-3.52 (m, 4 H), 3.13-3.01 (m 4 H), 2.22-2.09 (m, 2 H), 2.04-1.80 (m 4 H).

### EXAMPLE 320

### Preparation of 5-(3-Amino-propyl)-8-chloro-3-(2-ethoxy-ethyl)-2,5-dihydro-pyrazolo[4,3-c]quinolin-4-one

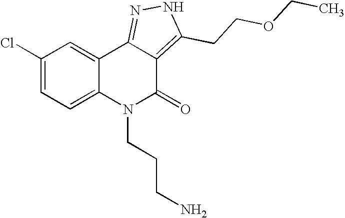

The title compound was synthesized from 3-(3-Benzyloxy-propionyl)-6-chloro-4-hydroxy-1H-quinolin-2-one, from Example 316, Step 3.

### Step 1: Preparation of 6-Chloro-3-(3-ethoxy-propionyl)-4-hydroxy-1H-quinolin-2-one

To a solution of 3-(3-Benzyloxy-propionyl)-6-chloro-4-hydroxy-1H-quinolin-2-one (447 mg, 1.25 mmol) in 50 mL ethanol was added a NaOEt/EtOH (25% by wt) solution. The reaction was heated for 1 h at 80° C. and then acidified with 1N HCl (aq) until a white precipitate was formed. The precipitate was filtered and dried to afford a white solid (340 mg, 92%). LCMS: Method FA, Rt=1.80 min, [MH−=293.97].

### Step 2: Preparation of 8-Chloro-3-(2-ethoxy-ethyl)-2,5-dihydro-pyrazolo[4,3-c]quinolin-4-one

This compound was made according to the procedure outlined in Example 202, Step 4 starting with 6-Chloro-3-(3-ethoxy-propionyl)-4-hydroxy-1H-quinolin-2-one (400 mg, 1.4 mmol) to form the desired product (232 mg, 57%). LCMS: Method FA, Rₜ=1.41 min, [MH+=291.99].

### Step 3: Preparation of 8-Chloro-3-(2-ethoxy-ethyl)-2-(tetrahydro-pyran-2-yl)-2,5-dihydro-pyrazolo[4,3-c]quinolin-4-one

This compound was made according to the procedure outlined in Example 202, Step 5 starting from 8-Chloro-3-(2-ethoxy-ethyl)-2,5-dihydro-pyrazolo[4,3-c]quinolin-4-one (232 mg, 0.797 mmol) to form the desired product (300 mg, quant).

### Step 4: Preparation of {3-[8-Chloro-3-(2-ethoxy-ethyl)-4-oxo-2-(tetrahydro pyran-2-yl)-2,4-dihydro-pyrazolo[4,3-c]quinolin-5-yl]-propyl}-carbamic acid tert-butyl ester

This compound was made according to the procedure outlined in Example 202, Step 6 starting with 8-Chloro-3-(2-ethoxy-ethyl)-2-(tetrahydro-pyran-2-yl)-2,5-dihydro-pyrazolo[4,3-c]quinolin-4-one (300 mg, 0.8 mmol) to form the desired product (97 mg, 23%). LCMS: Method FA, Rₜ=2.40 min, [MH+=533.15] [MNa+=555.15].

### Step 5: Preparation of 5-(3-Amino-propyl)-8-chloro-3-(2-ethoxy-ethyl)-2,5-dihydro-pyrazolo[4,3-c]quinolin-4-one

This compound was made according to the procedure outlined in Example 202, Step 7 starting with{3-[8-Chloro-3-(2-ethoxy-ethyl)-4-oxo-2-(tetrahydro-pyran-2-yl)-2,4-dihydro-pyrazolo[4,3-c]quinolin-5-yl]-propyl}-carbamic acid tert-butyl ester (45 mg, 0.08 mmol) to form the desired product (31 mg, quant). LCMS: Method FA, Rₜ=0.96 min, [MH+=349.12]. ¹H NMR 300 MHz (MeOD) δ 8.16-8.19 (m, 1H), 7.61-7.65 (m, 2H), 4.44-4.52 (m, 2H), 3.87 (t, 2H), 3.72 (t, 2H), 3.37 (m, 2H), 3.35-3.45 (m, 2H), 3.00-3.12 (m, 3H), 2.12-2.20 (m, 2H), 1.49-1.51 (m, 2H).

### EXAMPLE 321

### Preparation of 5-(3-Amino-propyl)-8-chloro-3-(2-propoxy-ethyl)-2,5-dihydro-pyrazolo[4,3-c]quinolin-4-one

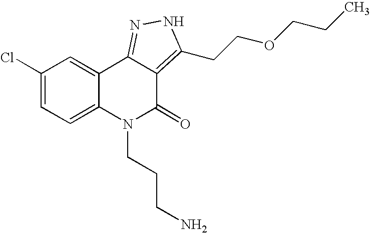

The title compound was prepared as in Example 320 from 3-(3-Benzyloxy-propionyl)-6-chloro-4-hydroxy-1H-quinolin-2-one.

### Step 1: Preparation of 6-Chloro-4-hydroxy-3-(3-propoxy-propionyl)-1H quinolin-2-one

Sodium metal (3 mL of a 30% by weight dispersion in toluene) was added slowly to 2 mL of 1-propanol. After gas evolution had ceased and the metal had dissolved in the solvent, 3-(3-Benzyloxy-propionyl)-6-chloro-4-hydroxy-1H-quinolin-2-one was added to the solution. The reaction was heated at 80° C. for 1 h. The reaction mixture was then diluted up with EtOAc and washed with 1N HCl (aq), NaCl (aq), dried with MgSO₄, filtered, and evaporated to form the desired product which was carried on crude to the next step without purification.

### Step 2: Preparation of 8-Chloro-3-(2-propoxy-ethyl)-2,5-dihydro-pyrazolo[4,3-c]quinolin-4-one

This compound was made according to the procedure outlined in Example 202, Step 4 starting with 6-Chloro-4-hydroxy-3-(3-propoxy-propionyl)-1H-quinolin-2-one to form the desired product (600 mg, quant). LCMS: Method FA, Rₜ=1.47 min, [MH+=306.08].

### Step 3: Preparation of 8-Chloro-3-(2-propoxy-ethyl)-2-(tetrahydro-pyran-2-yl)-2,5-dihydro-pyrazolo[4,3-c]quinolin-4-one

This compound was made according to the procedure outlined in Example 202, Step 5 starting with 8-Chloro-3-(2-propoxy-ethyl)-2,5-dihydro-pyrazolo[4,3-c]quinolin-4-one (521 mg, 1.7 mmol) to form the desired product (400 mg, 59%). LCMS: Method FA, Rₜ=2.12 min, [MH+=390.13].

### Step 4: Preparation of {3-[8-Chloro-4-oxo-3-(2-propoxy-ethyl)-2-(tetrahydro-pyran-2-yl)-2,4-dihydro-pyrazolo[4,3-c]quinolin-5-yl]-propyl}-carbamic acid tert-butyl ester

This compound was made according to the procedure outlined in Example 202, Step 6 starting with 8-Chloro-3-(2-propoxy-ethyl)-2-(tetrahydro-pyran-2-yl)-2,5-dihydropyrazolo[4,3-c]quinolin-4-one (400 mg, 1.0 mmol) to form the desired product (78 mg, 14%). LCMS: Method FA, Rₜ=2.54 min, [MH+=547.19] [MNa+=569.16].

### Step 5: Preparation of 5-(3-Amino-propyl)-8-chloro-3-(2-propoxy-ethyl)-2,5-dihydro-pyrazolo[4,3-c]quinolin-4-one

This compound was made according to the procedure outlined in Example 202, Step 7 starting with {3-[8-Chloro-4-oxo-3-(2-propoxy-ethyl)-2-(tetrahydro-pyran-2-yl)-2,4-dihydro-pyrazolo[4,3-c]quinolin-5-yl]-propyl}-carbamic acid tert-butyl ester (78 mg, 0.14 mmol) to form the desired product (43 mg, 83%). ¹H NMR 300 MHz (MeOD) δ 8.28-8.30 (m, 1H), 7.73-7.76 (m, 2H), 4.56-4.64 (m, 2H), 3.94-4.02 (m, 2H), 3.78-3.86 (m, 2H), 3.12-3.20 (m, 3H), 2.20-2.32 (m, 2H), 1.60-1.74 (m, 4H), 1.00 (t, 2H).

### EXAMPLE 322

### Preparation of 5-(3-Amino-propyl)-3-(2-butoxy-ethyl)-8-chloro-2,5-dihydro-pyrazolo[4,3-c]quinolin-4-one

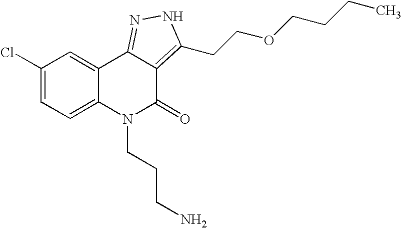

The title compound was prepared in a manner similar to Example 321 using appropriate reagents. LCMS: Method FA, Rₜ=1.16 min, [MH+=377.17]. ¹H NMR 300 MHz (MeOD) δ 8.24-8.28 (m, 1H), 7.69-7.74 (m, 2H), 4.51-4.61 (m, 2H), 3.90-3.98 (m, 2H), 3.53-3.61 (m, 2H), 3.41-3.49 (m, 2H), 3.07-3.16 (m, 2H), 2.18-2.30 (m, 2H), 1.54-1.68 (m, 2H), 1.35-1.47 (m, 2H), 0.93-1.01 (m, 3H).

### EXAMPLE 323

### Preparation of 5-(3-Amino-propyl)-8-chloro-3-[2-(3-methyl-butoxy)-ethyl]-2,5-dihydro-pyrazolo [4,3-cl quinolin-4-one

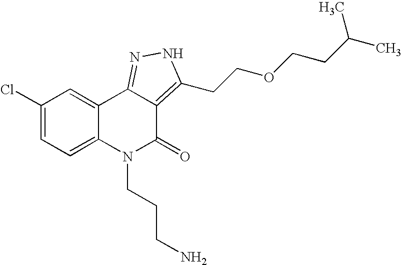

The title compound was prepared in a manner similar to Example 321 using appropriate reagents.

### EXAMPLE 324

### Preparation of 5-(3-Amino-propyl)-8-chloro-3-(2-methoxy-propyl)-2,5-dihydro-pyrazolo[4,3-c]quinolin-4-one

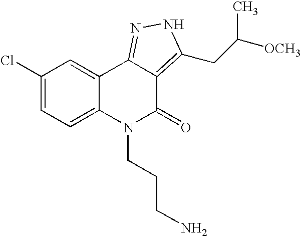

The title compound was prepared using methods outlined in Examples 202 and 311.

### Step 1: Preparation of 3-But-2-enoyl-6-chloro-4-hydroxy-1H-quinolin-2-one

This compound was prepared from 6-Chloro-4-hydroxy-2-oxo-1,2-dihydro-quinoline-3-carboxylic acid methoxy-methyl-amide using allylmagnesium chloride, and a procedure similar to that described in Example 311, Step 4. LCMS: ES⁺264.11 (M+1), ES⁻262.04 (M−1). ¹H NMR (300 MHz, DMSO) δ 11.62 (1 H, s), 7.93-7.87 (2 H,m), 7.72-7.68 1 H, m), 7.33-7.19 (2 H, m), 2.00 (3 H, d).

### Step 2: Preparation of 6-Chloro-4-hydroxy-3-(3-methoxy-butyryl)-1H-quinolin-2-one

This compound was made using the method outlined in Example 202, Step 3 starting with 3-But-2-enoyl-6-chloro-4-hydroxy-1H-quinolin-2-one(195 mg, 0.74 mmol) and sodium methoxide (4 mL of a 25% by weight solution in MeOH) to form the desired product which was carried on crude to the next step without purification. LCMS: Method FA, Rₜ=1.82 min, [MH+=296.05].

### Step 3: Preparation of 8-Chloro-3-(2-methoxy-propyl)-2,5-dihydro-pyrazolo[4,3-c]quinolin-4-one

This compound was made using the method outlined in Example 202, Step 4 starting with 6-Chloro-4-hydroxy-3-(3-methoxy-butyryl)-1H-quinolin-2-one to form the desired product which was carried on crude to the next step without purification. LCMS: Method FA, Rₜ=1.34 min, [MH+=292.12].

### Step 4: Preparation of 8-Chloro-3-(2-methoxy-propyl)-2-(tetrahydro-pyran-2-yl)-2,5-dihydro-pyrazolo[4,3-c]quinolin-4-one

This compound was made using the method outlined in Example 202, Step 5 starting with 8-Chloro-3-(2-methoxy-propyl)-2,5-dihydro-pyrazolo[4,3-c]quinolin-4-one to form the desired product which was carried on crude to the next step without purification. LCMS: Method FA, Rₜ=1.91 min, [MH+=376.15]

### Step 5: Preparation of {3-[8-Chloro-3-(2-methoxy-propyl)-4-oxo-2-(tetrahydro-pyran-2-yl)-2,4-dihydro-pyrazolo[4,3-c]quinolin-5-yl]-propyl}-carbamic acid tert-butyl ester

This compound was made using the method outlined in Example 202, Step 6 starting with 8-Chloro-3-(2-methoxy-propyl)-2-(tetrahydro-pyran-2-yl)-2,5-dihydro-pyrazolo-[4,3-c]quinolin-4-one to form the desired product (100 mg, 25% over 3 steps).

### Step 6: Preparation of 5-(3-Amino-propyl)-8-chloro-3-(2-methoxy-propyl)-2,5-dihydro-pyrazolo[4,3-c]quinolin-4-one

This compound was made using the method outlined in Example 202, Step 7 starting with {3-[8-Chloro-3-(2-methoxy-propyl)-4-oxo-2-(tetrahydro-pyran-2-yl)-2,4-dihydro-pyrazolo[4,3-c]quinolin-5-yl]-propyl}-carbamic acid tert-butyl ester (100 mg, 1.9 mmol) to form the desired product (70 mg, quant). LCMS: Method FA, Rₜ=0.96 min, [MH+=349.13]. ¹H NMR 300 MHz (MeOD) δ 8.30-8.33 (m, 1H), 7.72-7.77 (m, 2H), 4.60 (t, 2H), 3.90-4.05 (m, 2H), 3.75-3.88 (m, 2H), 3.10-3.20 (m, 3H), 2.20-2.30 (m, 2H), 1.90-2.00 (m, 1H), 1.60-1.70 (m, 2H), 1.31 (d, 3H).

### EXAMPLE 325

### Preparation of 5-(3-Amino-propyl)-8-chloro-3-isopropyl-2,5-dihydro-pyrazolo[4,3-c]quinolin-4-one

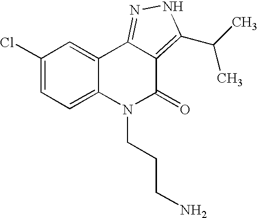

### Step 1: Preparation of 2-[3-(4-Chloro-phenylamino)-propyl]-isoindole-1,3-dione

To a solution of 3-(1,3-Dioxo-1,3-dihydro-isoindol-2-yl)-propionaldehyde (8.83 g, 43.45 mmol) in DCE (400 mL) was added 4-chloro-phenylamine (5.54 g, 43.45 mmol). After dissolution occurred, sodium triacetoxyborohydride (3.62 g, 17.066 mmol) and acetic acid (0.5 mL) were added and the reaction was heated to 50° C. and stirred 12 h. The reaction was cooled to ambient temperature and diluted with EtOAc. The organic solution was washed (sodium bicarbonate, water, brine) dried (MgSO₄), filtered, and concentrated in vacuo. The crude yellow cake was then crystallized from EtOAc/hexanes to afford 9.55 g of the title compound (yellow needles). LCMS: Method FA, Rₜ=1.99 min, [MH⁺=315.5]

### Step 2: Preparation of N-(4-Chloro-phenyl)-N-[3-(1,3-dioxo-1,3-dihydro-isoindol-2-yl)-propyl]-malonamic acid tert-butyl ester

To a solution of 2-[3-(4-chloro-phenylamino)-propyl]-isoindole-1,3-dione (1.90 g, 6.05 mmol) in DCM (60 mL) was added malonic acid mono-tert-butyl ester (0.984 mL, 6.66 mmol) and (3-dimethylamino-propyl)-ethyl-carbodiimide (1.26 g, 6.66 mmol). The reaction was stirred 1 h, transferred to a seperatory funnel, and washed (1 N HCl, sodium bicarbonate, brine), dried (MgSO₄), filtered, and concentrated in vacuo. Flash chromatography (gradient elution, 0-75% EtOAc/hexanes) provided 1.67 g of the title compound (white solid). LCMS: Method FA, Rₜ=2.08 min, [MH⁺=457.2]

### Step 3: Preparation of 2-[3-(6-Chloro-4-hydroxy-2-oxo-2H-quinolin-1-yl)-propyl]-isoindole-1,3-dione

To a solution of N-(4-chloro-phenyl)-N-[3-(1,3-dioxo-1,3-dihydro-isoindol-2-yl)-propyl]-malonamic acid tert-butyl ester (1.39 g, 3.05 mmol) in methanesulfonic acid (20 mL) was added phosphorous pentoxide (500 mg, 3.5 mmol). The reaction was heated (100° C.), stirred 1 h, then poured over 100 g ice. The white precipitate was filtered and dried in vacuo to afford 1.32 g of the title compound, a white solid. LCMS: Method FA, Rₜ=1.67 min, [MH⁺=383.1]

### Step 4: Preparation of 5-(3-Amino-propyl)-8-chloro-3-isopropyl-2,5-dihydro-pyrazolo[4,3-c]quinolin-4-one

To a suspension of 2-[3-(6-Chloro-4-hydroxy-2-oxo-2H-quinolin-1-yl)-propyl]-isoindole-1,3-dione (250 mg, 0.654 mmol) in pyridine (10 mL) was added isobutyryl chloride (100 uL, 0.943 mmol) and 4-dimethylaminopyridine (cat.). The reaction was sealed, heated (150° C.), and stirred. After 12 h the reaction was cooled and hydrazine (100 uL) was added. The reaction was again sealed, heated (150° C.) and stirred. After 1 h, the reaction was cooled and the white solid filtered. The mother liquors were then concentrated and purified via HPLC (gradient elution: acetonitrile containing zero to 100 percent 0.1% formic acid in water) to afford 54 mg of the title compound as a white powder. LCMS: Method FA, Rₜ=0.97 min, [MH⁺=319.1]; ¹HNMR (300 MHz, CD₃OD) δ 8.18 (s, 1 H), 7.60 (s, 2 H), 4.46 (dd, 2 H), 3.80-3.71 (m, 1 H), 2.99 (dd, 2 H), 2.18-2.09 (m, 2 H), 1.90 (s, 6H).

### EXAMPLE 326

### Preparation of 5-(3-Amino-propyl)-8-chloro-3-pyrazin-2-yl-ethyl)-2,5-dihydro-pyrazolo[4,3-c]quinolin-4-one

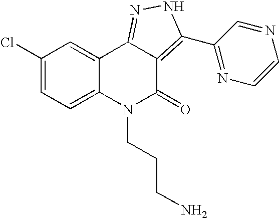

The title compound was prepared using the appropriate reagents in a manner similar to Example 325. LCMS: Method FA, Rₜ=1.36 min, [MH⁺=355.1]; ¹HNMR (300 MHz, CD₃OD) δ 8.83-8.77 (m, 2 H), 8.31 (s, 1 H), 8.10 (s, 1 H), 7.66-774 (m, 2 H), 3.07 (dd, 2 H), 2.11-2.25 (m, 2 H).

### EXAMPLE 327

### Preparation of 5-(3-Amino-propyl)-8-chloro-3-(tetrahydro-furan-3-yl)-2,5-dihydro-pyrazolo[4,3-c]quinolin-4-one

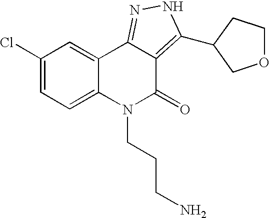

The title compound was prepared using the appropriate reagents in a manner similar to Example 325. LCMS: Method FA, Rₜ=0.93 min, [MH⁺=347.1]; ¹HNMR (300 MHz, CD₃OD) δ 8.16 (s, 1 H), 7.62 (s, 2 H), 4.46 (dd, 2 H), 4.25-4.17 (m, 1 H), 4.07-4.16 (m, 2 H), 3.92-4.00 (m, 2 H), 2.98 (dd, 2 H), 2.37-2.46 (m, 2 H), 2.07-2.18 (m, 2 H).

### EXAMPLE 328

### Preparation of 5-(3-Amino-propyl)-3-(2-hydroxy-ethyl)-8-(3-pyrrolidin-1-yl-prop-1-ynyl)-2,5-dihydro-pyrazolo [4,3-c]quinolin-4-one

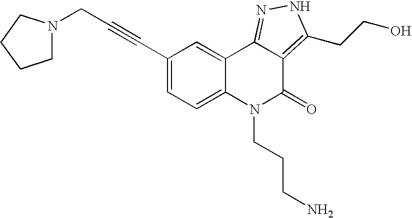

The title compound was prepared by methods outlined in Examples 285 and 316. ¹H NMR 300 MHz (MeOD) δ 8.31-8.36 (m, 1H), 7.72-7.80 (m, 2H), 4.45-4.60 (m, 2H), 4.09 (t, 2H), 3.81 (d, 2H), 3.40-3.50 (m, 2H), 2.85-2.95 (m, 4H), 1.95-2.05 (m, 6H), 1.55-1.75 (m, 2H).

### EXAMPLE 329

### Preparation of 5-(3-Amino-propyl)-3-ethyl-8-(3-pyrrolidin-1-yl-prop-1-ynyl)-2,5-dihydro-pyrazolo[4,3-c]quinolin-4-one

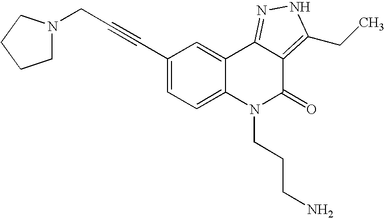

The title compound was prepared by methods outlined in Examples 285 and 311. ¹H NMR 300 MHz (MeOD) δ 8.45 (d, 1H), 7.85 (dd, 1H), 7.75 (d, 1H), 4.60 (t, 2H), 4.55 (s, 2H), 3.30-4.00 (m, 2H), 3.26 (q, 2H), 3.15 (t, 2H), 2.27 (t, 2H), 2.21-2.38 (m, 4H), 1.60-1.75 (m, 2H), 1.50 (t, 3H).

### EXAMPLE 330

### Preparation of 5-(3-Amino-propyl)-3-ethyl-8-(3-pyrrolidin-1-yl-propyl)-2,5-dihydro-pyrazolo[4,3-c]quinolin-4-one

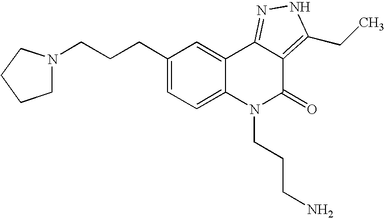

The title compound was prepared from Example 329 as in Example 294. ¹H NMR 300 MHz (MeOD) δ 8.22 (s, 1H), 7.69-7.78 (m, 2H), 4.61 (t, 2H), 3.73-3.89 (m, 4H), 3.33-3.48 (m, 2H), 3.25 (q, 2H), 3.14 (t, 2H), 3.02 (t, 2H), 2.10-2.38 (m, 8H), 1.51 (t, 3H).

### EXAMPLE 331

### Preparation of 8-Chloro-3-ethyl-5-(3-methylamino-propyl)-2,5-dihydro-pyrazolo[4,3-c]quinolin4-one

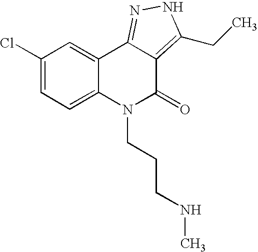

The title compound was prepared by methods outlined in Example 231 and 311. LCMS method FA, Rₜ=0.98, ES⁺319.16 (M+1). ¹H NMR (300 MHz, d₆ DMSO) δ 9.01 (2 H, br s), 8.25 (1 H, d), 7.69 (1 H, d), 7.62 (1 H, dd), 4.33 (2 H, t), 3.16 (3 H, s), 3.00 (2 H, q), 2.52 (2 H, t), 2.04-1.94 (2 H, m), 1.28 (3 H, t).

### EXAMPLE 332

### Preparation of 4-Chloro-N-{3-[3-methyl-4-oxo-8-(3-pyrrolidin-1-yl-prop-1-ynyl)-2,4-dihydro-pyrazolo[4,3-c]quinolin-5-yl]-propyl}-benzamide

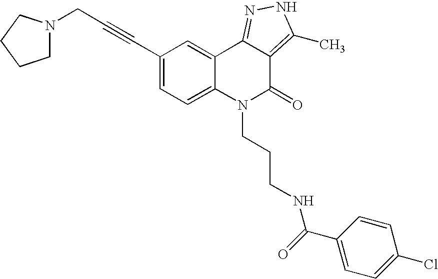

The title compound was prepared from Example 285.

To a solution of 5-(3-Amino-propyl)-3-methyl-8-(3-pyrrolidin-1-yl-prop-1-ynyl)-2,5-dihydro-pyrazolo[4,3-c]quinolin-4-one (60 mg, 0.165 mmol) in DCM (2 mL) was added triethylamine (0.034 mL, 0.248 mmol) and 4-chloro-benzoyl chloride (29 mg, 0.165 mmol). The reaction was stirred 30 min, concentrated, then purified by flash chromatography (gradient elution 0-10% MeOH in DCM, 1% NH₄OH). The resulting solid was triturated (MeOH) to afford 20 mg of the title compound (white solid). LCMS: Method FA, Rₜ=1.15 min, [MH⁺=502.2]; ¹HNMR (300 MHz, CD₃OD) δ 8.28 (s, 1 H), 7.82 (d, 2 H), 7.67 (dd, 1 H), 7.60 (d, 1 H), 7.48 (d, 2 H), 4.48-4.42 (m, 2 H), 4.42 (s, 2 H), 3.50 (dd, 2 H), 3.26-3.17 (m, 4 H), 2.68 (s, 3 H), 2.30-2.19 (m, 2 H), 2.14-2.00 (m, 4 H).

### EXAMPLE 333

### Preparation of 4-Chloro-N-{3-[3-methyl-4-oxo-8-(3-pyrrolidin-1-yl-propyl)-2,4-dihydro-pyrazolo [4,3-cl quinolin-5-yl]-propyl}-benzamide

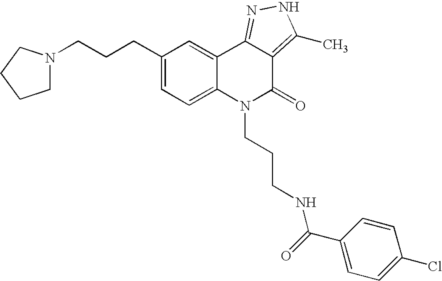

The title compound was prepared from Example 332.

To a solution of 4-Chloro-N-{3-[3-methyl-4-oxo-8-(3-pyrrolidin-1-yl-prop-1-ynyl)-2,4-dihydro-pyrazolo[4,3-c]quinolin-5-yl]-propyl}-benzamide (40 mg, 0.165 mmol) in MeOH (2 mL) was added 10% Pd on carbon (10 mg). The reaction was degassed and backfilled with Ar (3×). The reaction was stirred 30 min, filtered through a pad of celite (MeOH) and concentrated. Flash chromatography (gradient elution: 0-10% MeOH in DCM, 1% NH₄OH) afforded 40 mg of the title compound. LCMS: Method FA, Rₜ=1.15 min, [MH⁺=506.3]; ¹HNMR (300 MHz, CD₃OD) δ 8.01 (s 1 H), 7.83 (d, 2 H), 7.58-7.44 (m, 4 H), 4.49-4.39 (m, 2 H), 3.72-3.61 (m, 2 H), 3.53-3.45 (m, 2 H), 3.27-3.18 (m, 2 H), 3.12-3.02 (m, 2 H), 2.90-2.80 (m, 2 H), 2.67 (s, 3 H), 2.22-1.97 (m, 8 H).

### EXAMPLE 334

### Preparation of 5-(3-Amino-propyl)-3-methyl-4-oxo-4,5-dihydro-2H-pyrazolo[4,3-c]quinoline-7-carboxylic acid methyl ester

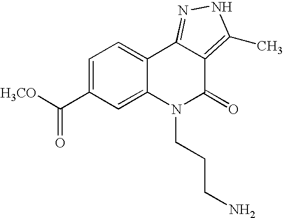

The title compound was prepared by methods similar to Example 202 starting with 2-Amino-terephthalic acid 1-methyl ester. LCMS: ES⁺315 (M+1). ¹H NMR 300 MHz (DMSO) δ 8.40-8.34 (1 H, m), 8.34-8.32 (1 H, m), 8.20-7.78 (1 H, m) 4.62 (2 H, t), 3.91 (3 H, s), 2.91 ( 2 H, t), 2.62 (3 H, s) 2.08-2.02 (2 H, m).

### EXAMPLE 335

### Preparation of 5-(3-Amino-propyl)-3-methyl-4-oxo-4,5-dihydro-2H-pyrazolo[4,3-c]quinoline-7-carboxylic acid (2-dimethylamino-ethyl)-amide

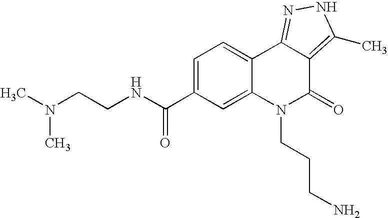

The title compound was prepared from 5-(3-tert-Butoxycarbonylamino-propyl)-3-methyl-4-oxo-2-(tetrahydro-pyran-2-yl)-4,5-dihydro-2H-pyrazolo[4,3-c]quinoline-7-carboxylic acid methyl ester, an intermediate from Example 334.

### Step 1: Preparation of 5-(3-tert-Butoxycarbonylamino-propyl)-3-methyl-4-oxo-2-(tetrahydro-pyran-2-yl)-4,5-dihydro-2H-pyrazolo[4,3-c]quinoline-7-carboxylic acid

To a solution of 5-(3-tert-Butoxycarbonylamino-propyl)-3-methyl-4-oxo-2-(tetrahydro-pyran-2-yl)-4,5-dihydro-2H-pyrazolo[4,3-c]quinoline-7-carboxylic acid methyl ester (0.55 g, 1.10 mmol) in a 1:1:3 solution of methanol, water, and tetrahydrofuran, was added 1 N NaOH (4.0 ml, 4.4 mmol). The reaction stirred at room temperature over night. The reaction was concentrated then diluted with dichloromethane and washed quickly with 1 N HCl. The organic fractions were combined, washed with brine, dried, (Na₂SO₄), and concentrated to yield the desired product. LCMS: ES⁺485 (M+1).

### Step 2: Preparation of 5-(3-Amino-propyl)-3-methyl-4-oxo-4,5-dihydro-2H-pyrazolo[4,3-c]quinoline-7-carboxylic acid (2-dimethylamino-ethyl)-amide

To a solution of 5-(3-tert-Butoxycarbonylamino-propyl)-3-methyl-4-oxo-2-(tetrahydro-pyran-2-yl)-4,5-dihydro-2H-pyrazolo[4,3-c]quinoline-7-carboxylic acid (0.20 g, 0.52 mmol) in dichloromethane was added diisopropylethyl amine (0.20 g, 1.56 mmol), N,N-dimethylethylenediamine (46 mg, 0.52 mmol) and HATU (0.22 g, 0.52 mmol). The reaction stirred at room temperature for 1 h. The reaction was concentrated and purified by chromatography on silica eluting with a mixture of dichloromethane 89%, methanol 10%, and NH₄OH 1% to yield the desired product.

### Step 3: Preparation of 5-(3-Amino-propyl)-3-methyl-4-oxo-4,5-dihydro-2H-pyrazolo[4,3-c]quinoline-7-carboxylic acid (2-dimethylamino-ethyl)-amide

The title compound was prepared by acidic deprotection as in Example 202, Step 7. ¹H NMR 300 MHz (MeOH) δ 8.44-8.40 (1H, m), 8.28 (1H, s), 8.03 (1H, d), 4.72 (2H, t), 4.01 (2H, t), 3.65-3.60 (2H, m), 3.25 (2H, t), 3.18 (6H, s), 2.86 (3H, s), 2.40-2.33 (2H, m). LCMS: ES⁺371 (M+1).

### EXAMPLE 336

### Preparation of 5-(3-Amino-propyl)-3-methyl-4-oxo-4,5-dihydro-2H-pyrazolo[4,3-c]quinolin-7-carboxylic acid (2-pyrrolidin-1-yl-ethyl)-amide

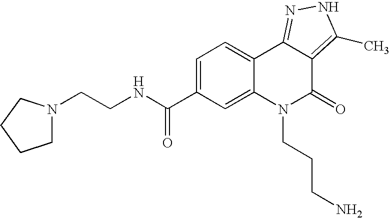

The title compound was prepared as in Example 335 using 2-Pyrrolidin-1-yl-ethylamine as the coupling partner. ¹H NMR 300 MHz (MeOH) δ 8.27-8.24 (1H, m), 8.20 (1H, s), 7.97-7.95 (1H, m), 4.64 (2H, t), 4.06-3.97 (4H, m), 3.68 (2H, t), 3.22 (2H, t), 2.80 (3H, s), 2.37-2.19 (8H, m). LCMS: ES⁺397 (M+1).

### EXAMPLE 337

### Preparation of 5-(3-Amino-propyl)-3-methyl-4-oxo-4,5-dihydro-2H-pyrazolo[4,3-c]quinolin-7-carboxylic acid (2-dimethylamino-propyl)-amide

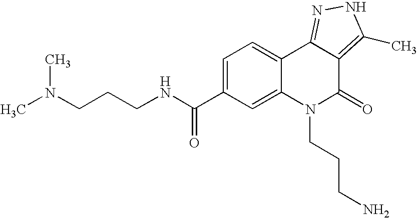

The title compound was prepared as in Example 335 using N,N-dimethyl-1,3-propanediamine as the coupling partner. ¹H NMR 300 MHz (MeOH) δ 8.37-8.34 (1H, m), 8.20 (1H, s), 7.98 (1H, d), 4.67 (2H, t), 3.70 (2H, t), 3.42-3.34 (2H, m), 3.18 (2H, t),3.10 ( 6H, s), 2.82 (3H, s), 2.39-2.19 (4H, m). LCMS: ES⁺385 (M+1).

### EXAMPLE 338

### Preparation of 5-(3-Amino-propyl)-3-methyl-4-oxo-4,5-dihydro-2H-pyrazolo[4,3-c]quinolin-7-carboxylic acid (2-dimethylamino-butyl)-amide

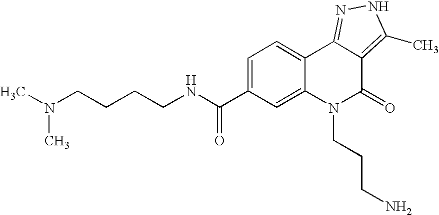

The title compound was prepared as in Example 335 using 4-dimethylaminobutylamine as the coupling partner. ¹H NMR 300 MHz (MeOH) δ 8.42-8.39 (1H, m), 8.21-8.20 (1H, m), 7.97-7.95 (1H, m), 4.70 (2H, t), 3.67 (2H, t), 3.40-3.34 (2H, m), 3.21 (2H, t), 3.06 (6H, s), 2.86 (3H, s), 2.40-2.31 (2H, m),2.05-1.88 (4H, m). LCMS: ES⁺399 (M+1).

### EXAMPLE 339

### Preparation of 5-(3-Aminopropyl)-7-iodo-3-methyl-2,5-dihydro-pyrazolo[4,3-c]quinolin4-one

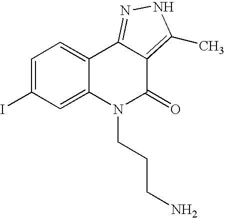

The title compound was prepared by methods similar to Example 202.

### Step 1: Preparation of N-(5-Iodo-2-methyl-phenyl)-acetamide

To a solution of 5-Iodo-2-methyl-phenylamine (15.0 g, 64.3 mmol) in dry methylene chloride at 0C was added acetic anhydride (13.4 g, 128.7 mmol) drop wise. The mixture was then heated to 50° C. for 1 hr. After cooling to room temperature, the white precipitate was filtered and washed with methylene chloride to yield the desired product. (16.1 g, 90%)

### Step 2: Preparation of 2-Acetylamino-4-iodo-benzoic acid

To a solution of N-(5-Iodo-2-methyl-phenyl)-acetamide (4.08 g, 14.8 mmo) in water was added potassium permanganate (7.03 g, 44.5 mmol) and magnesium sulfate (2.31 g, 19.24 mmol). The mixture was heated to reflux over night and then cooled to room temperature before filtering through a pad of celite. The filtrate was acidified with 1 N HCl and the white solid was collected to yield the desired product (2.95 g, 66%).

### Step 3: Preparation of 2-Amino-4-iodo-benzoic acid methyl ester

To a solution of 2-Acetylamino-4-iodo-benzoic acid (8.2 g, 26.8 mmol) in methanol at 0° was bubbled HCl gas for 10 minutes. The heated to reflux and stirred for 6 days. The mixture was cooled to room temperature and concentrated. The mixture was then dissolved in methylene chloride and extracted with 1 N NaOH (2×), washed with brine, dried over sodium sulfate and concentrated to yield the desired product (5.39 g, 72%).

### Step 4: Preparation of 5-(3-Aminopropyl)-7-iodo-3-methyl-2,5-dihydro-pyrazolo[4,3-c]quinolin-4-one

The title compound was prepared as in Example 202, Steps 2-7.

¹H NMR 300 MHz (MeOH) δ 8.11-8.10 (1H, m), 8.05-8.02 (1H, m), 7.88-7.85 (1H, m), 4.60 (2H, t), 3.17 (2H, t), 2.84 (3H, s), 2.34-2.24 ( 2H, m). LCMS: ES⁺383 (M+1).

### EXAMPLE 340

### Preparation of 5-(3-Amino-propyl)-3-methyl-7-(3-pyrrolidin-1-yl-prop-1-ynyl)-2,5-dihydro-pyrazolo [4,3-c]quinolin-4-one

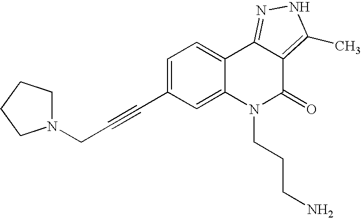

The title compound was prepared from {3-[7-Iodo-3-methyl-4-oxo-2-(tetrahydro-pyran-2-yl)-2,4-dihydro-pyrazolo[4,3-c]quinolin-5-yl]-propyl}-carbamic acid tert-butyl ester, an intermediate from Example 339 following methods outlined in Example 285. ¹H NMR 300 MHz (MeOH) δ 8.31-8.25 (1H, m), 7.94-7.90 (1H, m), 7.62-7.57 (1H, m), 4.62-4.55 (2H, m), 4.56-4.51 (2H, m), 3.74-3.55 (2H, m), 2.82-2.75 (3H, m), 2.32-2.19 (8H, m). LCMS: ES⁺364 (M+1).

### EXAMPLE 341

### Preparation of 5-(3-Amino-propyl)-3-methyl-7-(3-pyrrolidin-1-yl-propyl)-2,5-dihydro-pyrazolo[4,3-c]quinolin-4-one

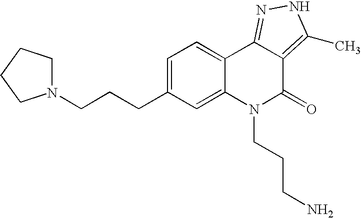

The title compound was prepared from Example 340 following methods outlined in Example 294. ¹H NMR 300 MHz (DMSO) δ 10.9-10.6 (1 H, m), 8.14-8.08 (1 H, m), 8.04-7.95 (2 H, m) 7.65-7.60 (1 H, m), 7.29-7.25 (1 H, m), 4.44-4.34 (2 H, m), 3.59-3.50 (2 H, m), 3.16-3.07 (2H, m), 3.04-2.82 (6H, m), 2.68-2.62 (1H, m), 2.50 (3H, s), 2.16-1.87 (8H, m). LCMS: ES⁺368 (M+1).

### EXAMPLE 342

### Preparation of 6-(3-amino-prop-1-ynyl)-8-chloro-3,5-dimethyl-2,5-dihydro-pyrazolo[4,3-c]quinolin-4-one

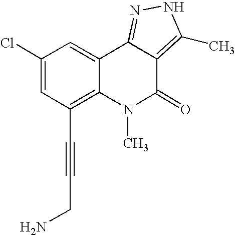

The title compound was prepared from 8-Chloro-3-methyl-2,5-dihydro-pyrazolo[4,3-c]quinolin-4-one, an intermediate from Example 209.

### Step 1: Preparation of 8-Chloro-3-methyl-6-nitro-2,5-dihydro-pyrazolo[4,3-c]quinolin-4-one

8-Chloro-3-methyl-2,5-dihydro-pyrazolo[4,3-c]quinolin-4-one (5 g, 21.5 mmol), was added portionwise to concentrated sulfuric acid (15 mL), then the mixture was cooled to 0° C. and KNO₃ (2.4 g, 23.6 mmol) was added portionwise. The mixture was stirred at 0° C. for 30 min, then allowed to warm to room temperature and stirred for 18 h. After this time the mixture was cooled again to 0° C. and KNO₃ (802 mg, 7.9 mmol) was added portionwise. The mixture was stirred at room temperature for 3 h, then ice was added and the mixture was poured into ice/water. The precipitate was filtered off, washed with water then hexane and dried under vacuum, to give the desired product as a yellow solid (6 g, 100%). LCMS: ES⁺279.09 (M+1), ES⁻277.06 (M−1).

### Step 2: Preparation of 8-Chloro-3-methyl-6-nitro-2-(tetrahydro-pyran-2-yl)-2,5-dihydro-pyrazolo[4,3-c]quinolin-4-one

This compound was prepared using standard conditions outlined in Example 202, Step 5. LCMS: ES⁺363.14 (M+1).

### Step 3: Preparation of 6-Amino-8-chloro-3,5-dimethyl-2-(tetrahydro-pyran-2-yl)-2,5-dihydro-pyrazolo[4,3-c]quinolin-4-one

To a suspension of compound 25 (6.8 g, 18.8 mmol) in DMF (200 mL) at 0° C., was added sodium hydride (60% suspension in oil, 1.13 g, 28.2 mmol) portionwise. The mixture was stirred at 0° C. for 10 min, then at room temperature for 50 min. Methyl iodide (2.34 mL, 37.6 mmol) was added dropwise and the reaction was stirred for 3 h. Water was added and the precipitate was filtered off, washed with water then hexane, and dried under vacuum. The solid was suspended in ethanol (200 mL) and Raney nickel (50% suspension in water, 2.5 g) was added. The mixture was hydrogenated at atmospheric pressure for 7 h. THF was added until the mixture was completely in solution, and then filtered through celite. The filtrate was evaporated and the residue was purified by chromatography on silica, eluting with 50% ethyl acetate/hexane then 100% ethyl acetate to give the desired compound as the more polar product (2.4 g, 37%).

### Step 4: Preparation of 6-Bromo-8-chloro-3,5-dimethyl-2,5-dihydro-pyrazolo[4,3-c]quinolin-4-one

To a suspension of compound 26 (1.5 g, 4.3 mmol) in DMF (10 mL) at 0° C., was added HBr (48% in water, 5 mL). A 0° C. solution of NaNO₂ (314 mg, 4.6 mmol) in water (3 mL) was added dropwise. The mixture was stirred at 0° C. for 15 min, and then it was added portionwise to a 100° C. solution of CuBr (373 mg, 2.6 mmol) in HBr (48% in water, 2 mL). The mixture was heated at 100° C. for 1 h, then cooled to room temperature and water added. The precipitate was filtered off and washed with water then hexane and dried under vacuum to give the desired product as a brown solid (1.5 g, quant). LCMS: ES⁺326.00 (M+1), ES⁻323.98 (M−1).

### Step 5: Preparation of 6-Bromo-8-chloro-3,5-dimethyl-2-(tetrahydro-pyran-2-yl)-2,5-dihydro-pyrazolo[4,3-c]quinolin-4-one

This compound was prepared using standard conditions outlined in Example 202, Step 6. LCMS: ES⁺410.05 (M+1).

### Step 6: Preparation of {3-[8-Chloro-3,5-dimethyl-4-oxo-2-(tetrahydro-pyran-2-yl)-4,5-dihydro-2H-pyrazolo[4,3-c]quinolin-6-yl]-prop-2-ynyl}-carbamic acid tert-butyl ester

This compound was prepared using methods outlined in Example 272. LCMS: ES⁺485.24 (M+1).

### Step 7: Preparation of 6-(3-Amino-prop-1-ynyl)-8-chloro-3,5-dimethyl-2,5-dihydro-pyrazolo[4,3-c]quinolin-4-one

Acidic deprotection as described in Example 202, Step 7 provided the title compound as a white solid. LCMS: FA, Rₜ=0.90 min, ES⁺301.11 (M+1). ¹H NMR (300 MHz, d₆ DMSO) δ 8.53 (1H, s), 7.69 (1H, s), 4.08 (2 H, s), 3.94 (3 H, s), 2.60 (3 H, s).

### EXAMPLE 343

### Preparation of 6-(3-Amino-propyl)-8-chloro-3,5-dimethyl-2,5-dihydro-pyrazolo[4,3-c]quinolin-4-one

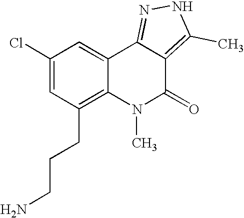

The title compound was prepared from Example 342 using hydrogenation conditions outlined in Example 294. LCMS: FA, Rₜ=0.92 min, ES⁺305.17 (M+1). ¹H NMR (300 MHz, MeOD) δ 8.13 (1 H, d), 7.61 (1 H, d), 3.81 (3 H, s), 3.28 (2 H. t), 3.04 (2 H, t), 2.79 (3 H, s), 2.11-2.00 (2 H, m).

### EXAMPLE 344

### Preparation of 8-Chloro-6-(3-hydroxy-prop-1-ynyl)-3,5-dimethyl-2,5-dihydro-pyrazolo[4,3-c]quinolin-4-one

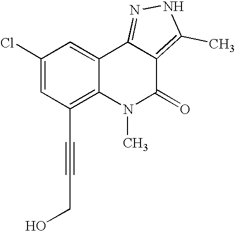

The title compound was prepared by methods outlined in Example 342. LCMS: FA Rₜ=1.35 min, ES⁺302.14 (M+1). ¹H NMR (300 MHz, d₆ DMSO) δ 8.09 (1 H, s), 7.60 (1 H, d), 5.37 (1 H, t), 4.35 (2 H, d), 3.89 (3 H, s), 2.55 (3 H, s).

### EXAMPLE 345

### Preparation of 6-(4-amino-but-1-ynyl)-8-chloro-3,5-dimethyl-2,5-dihydro-pyrazolo[4,3-c]quinolin-4-one

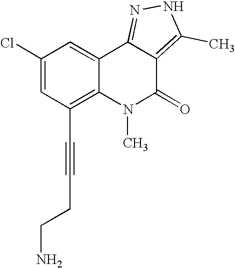

The title compound was prepared from 6-Bromo-8-chloro-3,5-dimethyl-2-(tetrahydro-pyran-2-yl)-2,5-dihydro-pyrazolo[4,3-c]quinolin-4-one, and intermediate from Example 342, and 2-But-3-ynyl-isoindole-1,3-dione by methods outlined in Example 342. To a solution of 2-{4-[8-Chloro-3,5-dimethyl-4-oxo-2-(tetrahydro-pyran-2-yl)-4,5-dihydro-2H-pyrazolo[4,3-c]quinolin-6-yl]-but-3-ynyl}-isoindole-1,3-dione (150 mg, 0.28 mmol) in ethanol (3 mL), was added hydrazine monohydrate (41 uL, 0.85 mmol). The mixture was heated under reflux for 4 hour, and then allowed to cool to room temperature, and the solvents evaporated. The residue was purified by chromatography on silica, eluting with 10% methanol/2% NH₄OH/88% dichloromethane, to give the desired product as an off-white solid (99 mg, 88%). LCMS: ES⁺399.21 (M+1).

The HCl salt of the title compound was prepared by acidic deprotection as described in Example 202, Step 7. LCMS: FA, Rₜ=0.99 min ES⁺315.14 (M+1). ¹H NMR (300 MHz, d₆ DMSO) δ 13.91 (1 H, Br s), 8.09 (1 H+2 H Br s), 7.77 (1 H, s), 3.91 (3 H, s), 3.08 (2 H, t), 2.86 (2 H, t), 2.56 (3 H, s).

### EXAMPLE 346

### Preparation of 6-(4-Amino-butyl)-8-chloro-3,5-dimethyl-2,5-dihydro-pyrazolo[4,3-c]quinolin-4-one

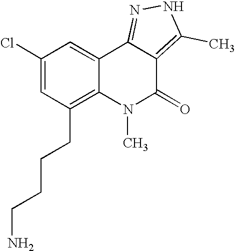

The title compound was prepared from Example 345 by methods outlined in Example 294. LCMS method FA, Rₜ=0.95, ES⁺319.19 (M+1). ¹H NMR (300 MHz, MeOD) δ 8.11 (1 H, d), 7.60 (1 H, d), 3.82 (3 H, s), 3.25 (2 H, t), 3.04 (2 H, t), 2.79 (3 H, s), 1.86-1.71 (4 H, m).

### EXAMPLE 347

### Preparation of 5-(3-Amino-propyl)-8-chloro-3-methyl-6-nitro-2,5-dihydro-pyrazolo[4,3-c]quinolin-4-one

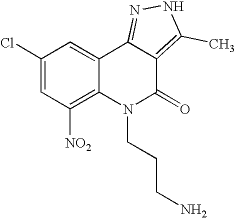

The title compound was prepared from 8-Chloro-3-methyl-2-(tetrahydro-pyran-2-yl)-2,5dihydro-pyrazolo[4,3-c]quinolin-4-one, an intermediate from Example 209.

### Step 1: Preparation of 2-{3-[8-Chloro-3-methyl-4-oxo-2-(tetrahydro-pyran-2-yl)-2,4-dihydro-pyrazolo[4,3-c]quinolin-5-yl]-propyl}-isoindole-1,3-dione

This compound was prepared from 8-Chloro-3-methyl-2-(tetrahydro-pyran-2-yl)-2,5-dihydro-pyrazolo[4,3-c]quinolin-4-one using 2-(3-Bromo-propyl)-isoindole-1,3-dione and a procedure similar to that described for Example 202, Step 6. LCMS: ES⁺505.16 (M+1).

### Step 2: Preparation of 2-[3-(8-Chloro-3-methyl-6-nitro-4-oxo-2,4-dihydro-pyrazolo[4,3-c]quinolin-5-yl)-propyl]-isoindole-1,3-dione

This compound was prepared from 2-{3-[8-Chloro-3-methyl-4-oxo-2-(tetrahydro-pyran-2-yl)-2,4-dihydro-pyrazolo[4,3-c]quinolin-5-yl]-propyl}-isoindole-1,3-dione using a procedure similar to that described for Example 342, Step 1. LCMS: ES⁺466.20 (M+1).

### Step 3: Preparation of 5-(3-Amino-propyl)-8-chloro-3-methyl-6-nitro-2,5-dihydro-pyrazolo[4,3-c]quinolin-4-one

The title compound was prepared from 2-[3-(8-Chloro-3-methyl-6-nitro-4-oxo-2,4-dihydro-pyrazolo[4,3-c]quinolin-5-yl)-propyl]-isoindole-1,3-dione using the procedure described for Example 345. LCMS: FA, Rₜ=1.01 min ES⁺336.14 (M+1). ¹H NMR (300 MHz, MeOH) δ 8.55 (1 H, m), 8.11 (1 H, m), 4.18 (2 H, t), 3.04 (2 H, t), 2.86 (3 H, s), 2.21 (2 H, t).

### EXAMPLE 348

### Preparation of 6-Amino-5-(3-amino-propyl)-8-chloro-3-methyl-2,5-dihydro-pyrazolo[4,3-c]quinolin4-one

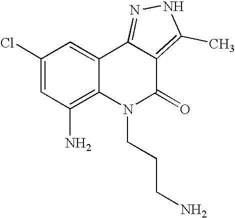

The title compound was prepared from 2-[3-(8-Chloro-3-methyl-6-nitro-4-oxo-2,4-dihydro-pyrazolo[4,3-c]quinolin-5-yl)-propyl]-isoindole-1,3-dione, an intermediate from Example 347 by methods described in Example 342. LCMS: FA, Rₜ=0.88 min, ES⁺306.16 (M+1). ¹H NMR (300 MHz, MeOH) δ 7.84 (1 H, d), 7.36 (1 H, d), 4.80 (2 H, t), 2.95 (2 H, t), 2.83 (3 H, s), 2.14-2.04 (2 H, m).

### EXAMPLE 349

### Preparation of 5-(3-Amino-propyl)-6-bromo-8-chloro-3-methyl 2,5-dihydro-pyrazolo[4,3-c]quinolin-4-one

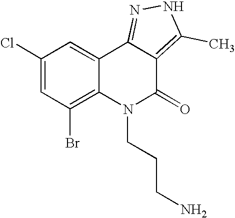

The title compound was prepared from 2-[3-(6-Amino-8-chloro-3-methyl-4-oxo-2,4-dihydro-pyrazolo[4,3-c]quinolin-5-yl)-propyl]-isoindole-1,3-dione, and intermediate from Example 348, by methods described in Example 342. LCMS: FA Rₜ=1.05 min, ES⁺369.11 (M+1). ¹H NMR (300 MHz, MeOH) δ 8.32 (1 H, d), 8.07 (1 H, d), 4.74 (2 H, t), 3.16 (2 H, t), 2.83 (3 H, s), 2.51-2.41 (2 H, m).

### EXAMPLE 350

### Preparation of 5-(3-Amino-propyl)-8-chloro-6-ethynyl-3-methyl-2,5-dihydro-pyrazolo[4,3-c]quinolin-4-one

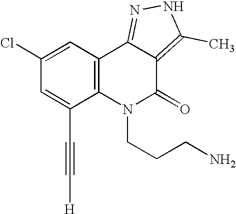

The title compound was prepared from 2-[3-(6-Bromo-8-chloro-3-methyl-4-oxo-2,4-dihydro-pyrazolo[4,3-c]quinolin-5-yl)-propyl]-isoindole-1,3-dione, an intermediate from Example 347.

### Step 1: Preparation of 2-{3-[6-Bromo-8-chloro-3-methyl-4-oxo-2-(tetrahydro-pyran-2-yl)-2,4-dihydro-pyrazolo[4,3-c]quinolin-5-yl]-propyl}-isoindole-1,3-dione

This compound was prepared as described in Example 202, Step 5. LCMS: ES⁺583.08 (M+1).

### Step 2: Preparation of 2-{3-[8-Chloro-3-methyl-4-oxo-2-(tetrahydro-pyran-2-yl)-6-trimethylsilanylethynyl-2,4-dihydro-pyrazolo[4,3-c]quinolin-5-yl]-propyl}-isoindole-1,3-dione

This compound was prepared from 2-{3-[6-Bromo-8-chloro-3-methyl-4-oxo-2-(tetrahydro-pyran-2-yl)-2,4-dihydro-pyrazolo[4,3-c]quinolin-5-yl]-propyl}-isoindole-1,3-dione as described in Example 291. LCMS: ES⁺601.15 (M+1).

### Step 3: Preparation of 2-{3-[8-Chloro-6-ethynyl-3-methyl-4-oxo-2-(tetrahydro-pyran-2-yl)-2,4-dihydro-pyrazolo[4,3-c]quinolin-5-yl]-propyl}-isoindole-1,3-dione

This compound was prepared from 2-{3-[8-Chloro-3-methyl-4-oxo-2-(tetrahydro-pyran-2-yl)-6-trimethylsilanylethynyl-2,4-dihydro-pyrazolo[4,3-c]quinolin-5-yl]-propyl}-isoindole-1,3-dione as described in Example 291. LCMS: ES⁺529.14 (M+1). Rt?

### Step 4: Preparation of 5-(3-Amino-propyl)-8-chloro-6-ethynyl-3-methyl-2-(tetrahydro-pyran-2-yl)-2,5-dihydro-pyrazolo[4,3-c]quinolin-4-one

This compound was prepared from 2-{3-[8-Chloro-6-ethynyl-3-methyl-4-oxo-2-(tetrahydro-pyran-2-yl)-2,4-dihydro-pyrazolo[4,3-c]quinolin-5-yl]-propyl}-isoindole-1,3-dione using the procedure described for Example 345. LCMS: ES⁺399.21 (M+1).

### Step 5: Preparation of 5-(3-Amino-propyl)-8-chloro-6-ethynyl-3-methyl-2,5-dihydro-pyrazolo[4,3-c]quinolin-4-one

The title compound was prepared from 5-(3-Amino-propyl)-8-chloro-6-ethynyl-3-methyl-2-(tetrahydro-pyran-2-yl)-2,5-dihydro-pyrazolo[4,3-c]quinolin-4-one as described in Example 202, Step 7. LCMS: ES⁺315.16 (M+1).

### EXAMPLE 351

### Preparation 5-Allyl-8-chloro-3-methyl-2,5-dihydro-pyrazolo[4,3-c]quinolin-4-one

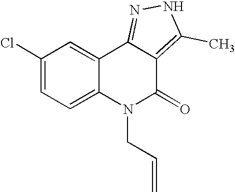

The title compound was prepared similarly to Example 204 using allyl bromide as the alkylating agent. ¹H NMR (300 MHz, CDCl₃) δ 8.11 (d, 1H), 7.44 (dd, 1H), 7.26 (1H, obscured 1H), 5.90-6.03 (m, 1H), 5.07-5.24 (m, 2H), 4.91-4.95 (m, 2H).

### EXAMPLE 352

### Preparation of 5-(3-Amino-propyl)-3-methyl-4-oxo-4,5-dihydro-2H-pyrazolo[4,3-c]quinoline-8-carboxylic acid (2-dimethylamino-ethyl)-amide

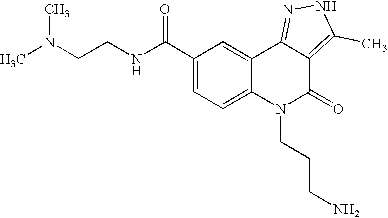

The title compound was prepared from 5-(3-tert-butoxycarbonylamino-propyl)-3-methyl-4-oxo-2-(tetrahydro-pyran-2-yl)-4,5-dihydro-2H-pyrazolo[4,3-c]quinoline-8-carboxylic acid, an intermediate from Example 266.

To a solution of 5-(3-tert-butoxycarbonylamino-propyl)-3-methyl-4-oxo-2-(tetrahydro-pyran-2-yl)-4,5-dihydro-2H-pyrazolo[4,3-c]quinoline-8-carboxylic acid (0.10 g, 0.21 mmol) in CH₂Cl₂ (2 mL) was added the amine (0.034 uL, 0.31 mmol), DIEA (0.1 mL, 0.62 mmol), and HATU (0.12 g, 0.31 mmol) and the mixture stirred for 48 h at 22° C. The reaction mixture was then concentrated in vacuo and purified by C-18 RP LC-MS chromatography to provide 0.075 g (0.14 mmol) of {3-[8-(2-dimethylamino-ethylcarbamoyl)-3-methyl-4-oxo-2-(tetrahydro-pyran-2-yl)-2,4-dihydro-pyrazolo[4,3-c]quinolin-5-yl]-propyl}-carbamic acid tert-butyl ester in 66% yield. LCMS: Method FA, Rₜ=1.16 min, [MH⁺=555.4].

The HCl salt of the title compound was prepared after deprotection as described above to give 0.045 g (0.12 mmol) of product for 86% yield. ¹H NMR 300 MHz (DMSO) δ 10.34-10.68 (bm, 1H), 9.03-9.21 (m, 1H), 8.83 (s, 1H), 8.18-8.33 (m, 1H), 7.99-8.17 (bm, 2H), 7.71-7.84 (m, 2H), 4.33-4.48 (m, 2H), 3.65-3.80 (m, 2H), 3.27-3.40 (m, 2H), 2.90-3.00 (m, 2H), 2.80-2.89 (m, 6H), 2.63 (s, 3H), 1.91-2.06 (m, 2H). LCMS: Method PFA, Rₜ=0.92 min, [MH⁺=371.3].

### EXAMPLE 353

### Preparation of 5-(3-Amino-propyl)-3-methyl-8-(2-pyridin-3-yl-ethyl)-2,5-dihydro-pyrazolo[4,3-c]quinolin-4-one

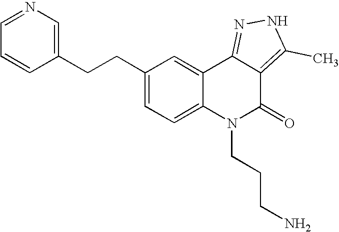

The title compound was prepared from Example 277 using methods similar to Example 294. ¹H NMR 300 MHz (DMSO) δ 8.91 (s, 1H), 8.81 (d, 1H), 8.51-8.57 (m, 1H), 8.00-8.12 (m, 3H), 7.43-7.62 (m, 2H), 4.34 (t, 2H), 3.08-3.27 (m, 4H), 2.84-2.94 (m, 2H), 2.60 (s, 3H), 1.93-2.03 (m, 2H).

### EXAMPLE 354

### Preparation of 5-(3-Amino-propyl)-3-(2-methoxy-ethyl)-8-methyl-2,5-dihydro-pyrazolo[4,3-c]quinolin-4-one

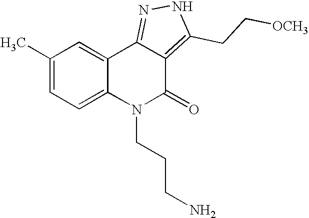

The title compound was prepared from the appropriate reagents by an analogous procedures to Example 202 and 205.

### EXAMPLE 355

### Preparation of 8-Chloro-5-(2,3-dihydroxy-propyl)-3-methyl-2,5-dihydro-pyrazolo[4,3-c]quinolin-4-one

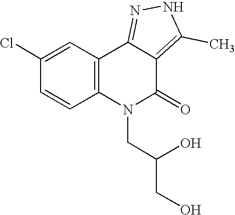

The title compound was prepared from 5-Allyl-8-chloro-3-methyl-2-(tetrahydro-pyran-2-yl)-2,5-dihydro-pyrazolo[4,3-c]quinolin-4-one, an intermediate in Example 351.

To a solution of 5-Allyl-8-chloro-3-methyl-2-(tetrahydro-pyran-2-yl)-2,5-dihydro-pyrazolo[4,3-c]quinolin-4-one (113 mg, 0.32 mmol) in THF (8 mL) and water (4.5 mL) was added OsO₄ (0.1 eq, 2.5 wt % tBuOH, tBuOOH, 0.03 mmol, 0.37 mL) and N-methyl morpholine N-oxide (45 mg, 0.38 mmol). After 24 hr, the reaction had not reached completion. An additional 0.3 mL of OsO₄ solution and 0.5 eq of the N-oxide was added. After 2 hr, the reaction was complete. A slurry of sodium hydrogen sulfite in water and florisil were added. After 30 min, the mixture was filtered and the solution was saturated with NaCl and extracted with EtOAc (3×). The combined organic layers were dried over MgSO₄, filtered and concentrated. ISCO chromatography provided the diol.

The diol was dissolved in DCM (3 mL) and a few drops of 4N HCl in dioxane was added. After 30 minutes, the precipated that had formed was filtered and washed with Et₂O to provide the title compound as a white solid. ¹H NMR 300 MHz (DMSO) δ 7.98 (d, 1H), 7.51 (d, 1H), 7.39 (dd, 1H), 4.38-4.46 (m, 1H), 3.94-4.17 (m, 2H), 3.41-3.54 (m, 2H), 2.60 (s, 3H).

### EXAMPLE 356

### Preparation of 8-Chloro-5-(3,4-dihydroxy-butyl)-3-methyl-2,5-dihydro-pyrazolo[4,3-c]quinolin-4-one

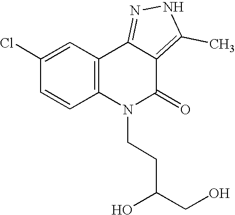

The title compound was prepared from 5-But-3-enyl-8-chloro-3-methyl-2-(tetrahydro-pyran-2-yl)-2,5-dihydro-pyrazolo[4,3-c]quinolin-4-one, an intermediate in Example 210. ¹H NMR 300 MHz (DMSO) δ 8.19 (s, 1H), 7.65 (s, 2H), 4.28-4.44 (m, 2H), 3.54-3.64 (m, 1H), 3.27-3.44 (m, 2H), 2.62 (s, 3H), 1.77-1.89 (m, 1H), 1.52-1.65 (m, 1H).

### EXAMPLE 357

### Preparation of 3-Amino-N-[5-(3-amino-propyl)-3-methyl-4-oxo-4,5-dihydro-2H-pyrazolo[4,3-c]quinolin-8-yl]-propionamide

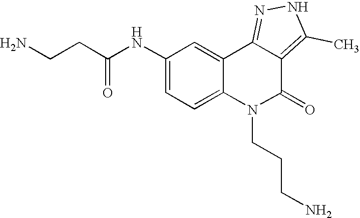

The title compound was prepared by analogous methods to Example 262.

### EXAMPLE 358

### Preparation of 5-(3-Amino-propyl)-3-methyl-8-pyridin-4-yl-2,5-dihydro-pyrazolo[4,3-c]quinolin-4-one

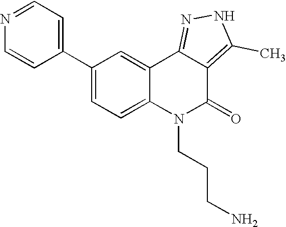

The title compound was prepared from {3-[8-Bromo-3-methyl-4-oxo-2-(tetrahydro-pyran-2-yl)-2,4-dihydro-pyrazolo[4,3-c]quinolin-5-yl]-propyl}-carbamic acid tert-butyl ester as in Example 306. LCMS: Method FA, Rₜ=0.72 min, [MH⁺=334.2]; ¹HNMR (300 MHz, CD₃OD) δ 8.69-8.75 (m, 3 H), 8.10 (d, 1 H), 7.93 (d, 2 H), 7.80 (d, 1 H), 4.53 (dd, 2 H), 3.06 (dd, 2 H), 2.70 (s, 3 H), 2.26-2.15 (m, 2 H).

### EXAMPLE 359

### Preparation of 5-(3-Amino-propyl)-3-(2-methoxy-ethyl)-8-pyrrolidin-1-yl-2,5-dihydro-pyrazolo [4,3-c]quinolin-4-one

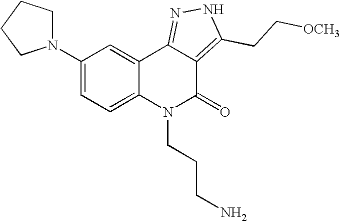

The title compound was made from Example 56 by methods outlined in Example 120.

### EXAMPLE 360

### Preparation of 5-(3-Amino-propyl)-3-ethyl-8-ethynyl-2,5-dihydro-pyrazolo[4,3-c]quinolin-4-one

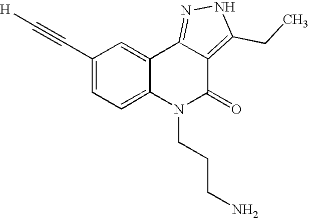

The title compound was prepared via methods outlined in Example 291 and 311. ¹H NMR 300 MHz (MeOD) δ 8.40 (d, 1H), 7.80 (dd, 1H), 7.71 (d, 1H), 4.59 (t, 2H), 3.71 (s, 1H), 3.23 (q, 2H), 3.12 (t, 2H), 2.81 (bs, 1H), 2.26 (t, 1H), 1.49 (t, 3H).

### EXAMPLE 361

### Preparation of 5-(3-Amino-propyl)-8-chloro-3-(3-methoxy-propyl}2,5-dihydro-pyrazolo[4,3-c]quinolin-4-one

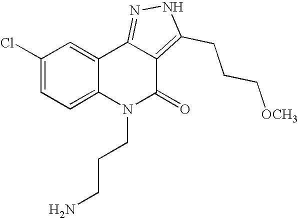

The title compound was prepared by methods outlined in Example 314. LCMS method FA, Rₜ=0.95, ES⁺349.15 (M+1). ¹H NMR (300 MHz, MeOD) δ 8.17 (1 H, d), 7.64-7.62 (2 H, m), 4.47 (2 H, t), 3.48 (2 H, t), 3.35 (3 H, s), 3.18 (2 H, t), 3.03 (2 H, t), 2.14-2.04 (4 H, m).

### EXAMPLE 362

### Preparation of 5-(3-Amino-propyl)-3-(2-benzyloxy-ethyl)-8-chloro-2,5-dihydro-pyrazolo[4,3-c]quinolin-4-one

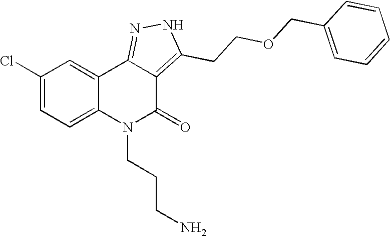

This compound was made according to procedures outlined in Example 316.

¹H NMR 300 MHz (MeOD) δ 8.09 (s, 1H), 7.55-7.59 (m, 2H), 7.08-7.16 (m, 5H), 4.45 (s, 2H), 4.30-4.40 (m, 2H), 3.85 (t, 2H), 3.33 (t, 2H), 2.90-2.99 (m, 2H), 2.00-2.10 (m, 2H).

### EXAMPLE 363

### Preparation of 8-Ethyl-3-(2-hydroxy-ethyl)-5-(3-hydroxy-propyl)-2,5-dihydro-pyrazolo[4,3-c]quinolin-4-one

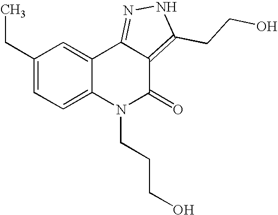

The title compound was prepared by methods outlined in Examples 294 and 316. ¹H NMR 300 MHz (MeOD) δ 8.16 (s, 1H), 7.65-7.72 (m, 2H), 4.63 (t, 2H), 4.11 (t, 2H), 3.40-3.49 (m, 2H), 3.13 (t, 2H), 2.93 (q, 2H), 2.25-2.35 (m, 2H), 1.46 (t, 3H).

### EXAMPLE 364

### Preparation of 5-(3-Amino-propyl)-8-chloro-3-propyl-2,5-dihydro-pyrazolo[4,3-c]quinolin-4-one

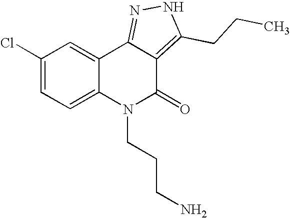

The title compound was prepared using the appropriate reagents in a manner similar to Example 325. LCMS: Method FA, Rₜ=0.97 min, [MH⁺=319.1]; ¹HNMR (300 MHz, C₂D₆SO) δ 8.20 (s, 1 H), 7.70-7.59 (m, 2 H), 4.33 (dd, 2 H), 2.97 (dd, 2 H) 2.92-2.83 (m, 2 H), 1.98-1.89 (m, 2 H), 1.79-1.68 (m, 2 H), 0.92 (t, 3 H).

### EXAMPLE 365

### Preparation of 5-(3-Amino-propyl)-8-chloro-3-cyclopentyl-2,5-dihydro-pyrazolo[4,3-c]quinolin4-one

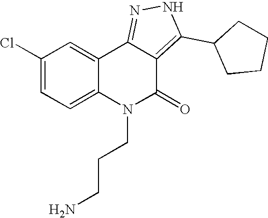

The title compound was prepared using the appropriate reagents in a manner similar to Example 325. LCMS: Method FA, Rₜ=1.079 min, [MH⁺=345.2]; ¹HNMR (300 MHz, CD₃OD) δ 8.17 (m, 1 H), 7.62-7.56 (m, 2 H), 4.45 (dd, 2 H), 3.82-3.71 (m, 1 H), 3.00 (dd, 2 H), 2.19-1.69 (m, 10 H).

### EXAMPLE 366

### Preparation of 5-(3-Amino-propyl)-3-methyl-4-oxo-4,5-dihydro-2H-pyrazolo[4,3-c]quinoline-7-carboxylic acid (2-pyridin-3-yl-ethyl)-amide

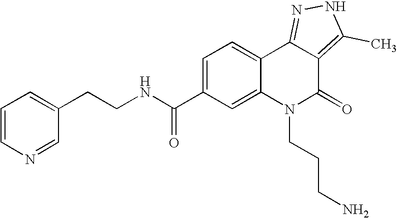

The title compound was prepared as in Example 335 using 3-(2-aminoethyl)pyridine as the coupling partner. ¹H NMR 300 MHz (MeOH) δ 8.98 (1H, s), 8.87-8.84 (1H, m), 8.75-8.71 (1H, m), 8.34-8.31 (1H, m), 8.18-8.08 (2H, m), 7.86-7.81 (1H, m) 4.63 (2H, t), 3.92 (2H, t), 3.37-3.32 (2H, m), 3.14 (2H, t), 2.79 (3H, s), 2.33-2.23 (2H, m). LCMS: ES⁺405 (M+1).

### EXAMPLE 367

### Preparation of 8-Chloro-6-(3-hydroxy-propyl)-3,5-dimethyl-2,5-dihydro-pyrazolo[4,3-c]quinolin-4-one

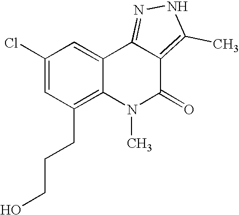

The title compound was prepared from Example 344 by methods outlined in Example 294.

### EXAMPLE 368

### Preparation of N-[5-(3-Amino-propyl)-3-methyl-4-oxo-4,5-dihydro-2H-pyrazolo[4,3-c]quinolin-8-yl]-3-diethylamino-propionamide

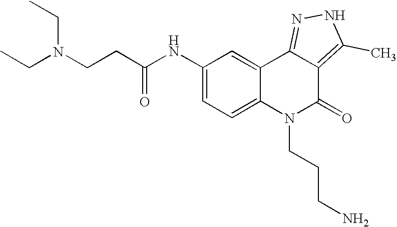

The title compound was prepared as in Example 262. ¹H NMR (300 MHz, DMSO-d₆) δ 10.7 (s, 1H), 10.5 (br s, 1H), 8.50 (d, J=2.1 Hz, 1H), 8.00 (br s, 3H), 7.54-7.79 (m, 2H), 4.31 (t, J=6.3 Hz, 2H), 3.37 (dd, J=6.8, 11.8 Hz, 2H), 3.08-3.21 (m, 4H), 2.96 (t, J=7.3 Hz, 2H), 2.80-2.93 (m, 2H), 2.59 (s, 3H), 1.89-2.02 (m, 2H), 1.26 (t, J=7.2 Hz, 6H) ppm; LC/MS: AA standard Rₜ=0.90 min, EI⁺ 399.25.

### EXAMPLE 369

### Preparation of N-[5-(3-Amino-propyl)-3-methyl4-oxo-4,5-dihydro-2H-pyrazolo[4,3-c]quinolin-8-yl]-3-piperidin-1-yl-propionamide

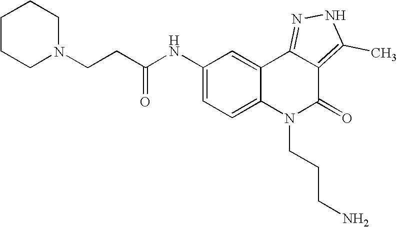

The title compound was prepared as in Example 262. ¹H NMR (300 MHz, DMSO-d₆) δ 10.3 (s, 1H), 8.47 (s, 1H), 7.51-7.67 (m, 2H), 4.27 (t, J=6.6 Hz, 2H), 2.68 (t, J=6.7 Hz, 2H), 2.56-2.65 (m, 5H), 2.45-2.56 (m, 2H), 2.34-2.45 (m, 4H), 1.85 (s, 6H), 1.70-1.82 (m, 2H), 1.45-1.57 (m, 4H), 1.32-1.45 (m, 2H) ppm; LC/MS: AA standard Rₜ=1.02 min, EI⁺ 411.22.

While this invention has been particularly shown and described with references to preferred embodiments thereof, it will be understood by those skilled in the art that various changes in form and details may be made therein without departing from the scope of the invention encompassed by the appended claims.

1. A compound represented by the following structural formula: 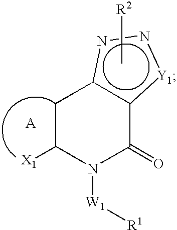
or a pharmaceutically acceptable salt thereof, wherein:
Ring A is a monocyclic aromatic group that is optionally substituted at any one or more substitutable ring atoms and is optionally fused to a second monocyclic aromatic group, Ring B;
Ring B is optionally substituted at any one or more substitutable ring atoms;
Y₁ is N or CR³;
R¹ is —H, —CONR¹¹R¹², —COOR¹², —C(═NR¹¹)—NR¹¹R¹², an optionally substituted heteroaryl group, an optionally substituted non-aromatic heterocyclic group, and W₁ is a linear C1-C6 alkylidene chain; R¹ is —OR¹², —NR¹¹R¹², —CN, —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —O—C(O)—OR¹², —OC(O)—NR¹¹R¹², —NR¹¹CO—CH(OR^{12a})—R¹², —NR¹¹CO—CH(N^{12a}R^{12a})—R¹², —NR¹¹CO—(CH₂)ₙCH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c)—NR}¹¹R¹², —NR¹¹—C(R¹²)—C(O)OR¹², —NR¹¹—C(R¹²)—C(O)NR¹¹R¹², —NR¹¹—C(R¹²)CH₂OR¹², cycloalkyl or -Ph and W₁ is a linear C2-C6 alkylidene group; or —W₁—R¹ is —H; wherein the alkylidene group represented by W₁ is optionally monosubstituted with —OR^{12b}, —N(R^{12b})₂, oxo, halo, or a spiro cycloalkyl group and wherein the alkylidene group represented by W₁ is optionally substituted with one or more —CH₃ groups, provided that the alkylidene group represented by W₁ is monosubstituted with —OR^{12b} or —N(R^{12b})₂ when R¹ is cycloalkyl or -Ph; and
R² is —H or a group that is cleavable in vivo;
R³ is —H, halogen, alkyl, haloalkyl or —V₁—R^{3a}, wherein V₁ is a covalent bond or a C1-C4 alkylidene optionally substituted with one or more —OR^{a}, —NR^{b}R^{c}, alkyl, hydroxyalkyl, alkoxyalkyl, aminoalkyl, or with a spiro cycloalkyl group; R^{3a} is —OR^{a}, —SR^{a}, —CONR^{b}R^{c}, —NR^{b}R^{c}, —NHC(O)NR^{a}R^{b}, —CN, —COOH, —COOR^{a}, —NHC(O)H, —NHC(O)R^{a}, —OC(O)R^{a}, —OC(O)NR^{b}R^{c}, —NHC(O)—OR^{a}, —S(O)₂NR^{b}R^{c}, —S(O)₂(R^{a}), boronate, alkyl boronate, —C(═NR^{a})—NR^{b}R^{c}, —NH—C(═NR^{a})NR^{b}R^{c}, —NH—C(═NR^{a})R^{a}, an optionally substituted cycloaliphatic or non-aromatic heterocyclic group, or an optionally substituted aromatic or aralkyl group; R^{a} is —H, alkyl or an optionally substituted aromatic or aralkyl group; and R^{b} and R^{c} are independently —H, alkyl or an optionally substituted aromatic or aralkyl group; or —NR^{b}R^{c} is an optionally substituted nitrogen-containing non-aromatic heterocyclic group;
X₁ is O, S, N, or CR⁴ when R¹ is —CONR¹¹R¹², —COOR¹², —C(═NR¹¹)—NR¹¹R¹², an optionally substituted heteroaryl group, an optionally substituted non-aromatic heterocyclic group, —OR¹², —NR¹¹R¹², —CN, —NR¹¹CONR¹¹R¹², —NR¹¹COR¹², —NH—C(═NR¹¹)NR¹¹R¹², —N═C(NR¹¹R¹²)₂, —SO₂NR¹¹R¹², —NR¹¹SO₂R¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —NR¹¹CO—(CH₂)ₙCH(NR^{12a}R^{12a})—R¹², —OC(O)—CH(OR^{12a})—R¹², —OC(O)—CH(NR^{12a}R^{12a})—R¹², —NR¹¹CO(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —NR¹¹—C(R¹²)—C(O)OR¹², —NR¹¹—C(R¹²)—C(O)NR¹¹R¹², —NR¹¹—C(R¹²)CH₂OR¹², cycloalkyl or -Ph; and X₁ is C—W₂—R⁵ when R₁ is —H and when —W₁—R¹ is —H;
W₂ is a linear C1-C6 alkylidene chain, optionally monosubstituted with —OR^{12b}, —N(R^{12b})₂, or a spiro cycloalkyl group or with one or more —CH₃ groups; wherein the C1-C6 alkylidene group represented by W₂ optionally has a cyclopropyl group, a monomethylated cyclopropyl group or dimethylated cyclopropyl group fused thereto; and wherein one carbon atom in the C1-C6 alkylidene group represented by W₂ is optionally replaced with T;
T is a covalent bond, —C≡C—, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—;
R⁴ is —H, C1-C3 alkyl, C1-C3 haloalkyl, halogen, hydroxy, C1-C3 alkoxy, C1-C3 haloalkoxy, —NH₂, C1-C3 alkylamine, C1-C3 dialkylamine, —NHC(O)H, —NHC(O)(C1-C3 alkyl), —C(O)NH₂, —C(O)NH(C1-C3 alkyl) or —C(O)N(C1-C3 alkyl)₂;
R⁵ is an optionally substituted heteroaryl group, an optionally substituted non-aromatic heterocyclic group, —OR¹², —NR¹¹R¹², —CN, —NR¹¹CONR¹¹R¹², —NR¹¹SO₂R¹², —NR¹¹COR¹², —NH—C(═NR¹¹)NR¹¹R¹², —SO₂NR¹¹R¹², —CONR¹¹R¹², —COOR¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², —CH(NR¹¹R¹²)-Ph, —CH(NR¹¹R¹²)-(cycloalkyl), a cycloalkyl group or a phenyl group substituted with —V₂—OR¹², —V—NR¹¹R¹², wherein V₂ is a covalent bond or a C1-C5 alkylene group;
R is —H or C1-C3 alkyl;
each R¹¹ is independently —H or a C1-C3 alkyl group; and
each R¹² is independently —H, an optionally substituted alkyl, aromatic, aralkyl, non-aromatic heterocyclic or non-aromatic heterocyclylalkyl group; or —NR¹¹R¹² is an optionally substituted non-aromatic nitrogen-containing heterocyclic group;
each R^{12a} is independently —H, a C1-C3 alkyl group, —C(O)H, —C(O)—(C1-C3 alkyl), —C(O)NH₂, —C(O)NH—(C1-C3 alkyl), —C(O)N—(C1-C3 alkyl)₂, —C(O)O—(C1-C3 alkyl), —S(O)₂(C1-C3 alkyl) or —NR^{12a}R^{12a} taken together is a substituted or unsubstituted non-aromatic nitrogen-containing heterocyclic group;
each R^{12b} is independently —H or a C1-C3 alkyl group or —NR^{12b}R^{12b} taken together is a substituted or unsubstituted non-aromatic nitrogen-containing heterocyclic group;
each R^{12c} is independently —H, a C1-C3 alkyl group or —(R^{12c}R^{12c})— taken together is a C3-C8 cycloalkyl group;
Ph is an optionally substituted phenyl group; and
n is an integer from 1 to 4.

2. The compound of claim 1 wherein:
R¹ is —H, —CONR¹¹R¹², —COOR¹², an optionally substituted heteroaryl group, an optionally substituted non-aromatic heterocyclic group, and W₁ is a linear C1-C6 alkylidene chain; R¹ is —OR¹², —NR¹¹R¹², —CN, —NR¹¹CONR¹¹R¹², —NR¹¹COR¹², —NH—C(═NR¹¹)NR¹¹R¹², —SO₂NR¹¹R¹², —NR¹¹SO₂R¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², cycloalkyl or -Ph and W₁ is a linear C2-C6 alkylidene group; or —W₁—R¹ is —H; wherein the alkylidene group represented by W₁ is optionally monosubstituted with —OR^{12b}, —N(R^{12b})₂, or a spiro cycloalkyl group and wherein the alkylidene group represented by W₁ is optionally substituted with one or more —CH₃ groups, provided that the alkylidene group represented by W₁ is monosubstituted with —OR^{12b} or —N(R^{12b})₂ when R¹ is cycloalkyl or -Ph;
R³ is —H, halogen, alkyl, haloalkyl or —V₁—R^{3a}, wherein V₁ is a covalent bond or a C1-C4 alkylidene optionally substituted with one or more methyl groups or with a spiro cycloalkyl group; R^{3a} is —OR^{a}, —SR^{a}, —CONR^{b}R^{c}, —NR^{b}R^{c}, —NHC(O)NR^{a}R^{b}, —CN, —COOH, —COOR^{a}, —NHC(O)H, —NHC(O)R^{a}, —OC(O)R^{a}, —OC(O)NR^{b}R^{c}, —NHC(O)—OR^{a}, boronate, alkyl boronate, or an optionally substituted aromatic or aralkyl group; and
T is a covalent bond, —O—, —S—, —N(R⁶)—, —S(O)—, —SO₂—, —C(O)—, —OC(O)—, —C(O)O—, —N(R⁶)C(O)—, —C(O)N(R⁶)—, —SO₂N(R⁶)—, or —N(R⁶)SO₂—.

3. The compound of claim 2 wherein the compound is represented by the following structural formula: 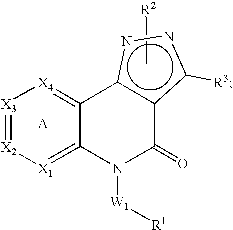
wherein:
X₁ is N, or CR⁴ when R¹ is —CONR¹¹R¹², —COOR¹², an optionally substituted heteroaryl group, an optionally substituted non-aromatic heterocyclic group, —OR¹², —NR¹¹R¹², —CN, —NR¹¹CONR¹¹R¹², —NR¹¹SO₂R¹², —NR¹¹COR¹², —NH—C(═NR¹¹)NR¹¹R¹², —SO₂NR¹¹R¹², —OC(O)R¹², —NR¹¹C(O)OR¹², —OC(O)—NR¹¹R¹², —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{12c}R^{12c})—OR¹², —OC(O)—C(R^{12c}R^{12c})—NR¹¹R¹², cycloalkyl or -Ph; and X₁ is C—W₂—R⁵ when R₁ is —H;
X₂, X₃ and X₄ are independently N or CH, provided that Ring A is not a tetrazole or a 1,2,3-triazole, provided that Ring A is optionally substituted at any one or more substitutable ring carbon atoms and provided that Ring A is optionally fused to a phenyl ring, Ring C, that is optionally substituted at any one or more substitutable ring carbon atoms.

4. The compound of claim 3 wherein the compound is represented by the following structural formula: 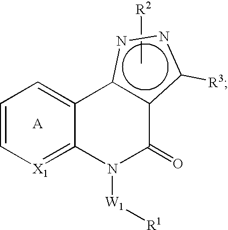
wherein:
Ring A is optionally substituted at any one or more substitutable ring carbon atoms and is optionally fused to a phenyl group, Ring C; and
Ring C is optionally substituted at any one or more substitutable ring carbon atoms.

5. The compound of claim 4 wherein:
Ring A and Ring C are optionally and independently substituted at any one or more substitutable ring carbon atoms with a substituent selected from halogen, R^{o}, —OR^{o}, —O(haloalkyl), —SR^{o}, 1,2-methylene-dioxy, 1,2-ethylenedioxy, trialkylsilyl, boronate, alkylboronate, dialkylboronate, —NO_{2,} —CN, —N(R′)₂, —NR′CO₂R^{o}, —NR′C(O)R^{o}, —NR′NR′C(O)R^{o}, —N(R′)C(O)N(R′)₂, —NR′NR′C(O)N(R′)₂, —NR′NR′CO₂R^{o}, —C(O)C(O)R^{o}, —C(O)CH₂C(O)R^{o}, —CO₂R^{o}, —C(O)R^{o}, —C(O)N(R^{o})₂, —OC(O)R^{o}, —OC(O)N(R^{o})₂, —S(O)₂R^{o}, —SO₂N(R′)₂, —S(O)R^{o}, —NR′SO₂N(R′)₂, —NR′SO₂R^{o}, —C(═S)N(R′)₂, or —C(═NH)—N(R′)₂;
each R′ is independently R^{o}, —CO₂R^{o}, —SO₂R^{o} or —C(O)R^{o} or —NR′R′ is an optionally substituted non-aromatic nitrogen-containing heterocyclic group;
each R^{o} is independently hydrogen or an alkyl group, non-aromatic heterocyclic group or aromatic group and the alkyl, non-aromatic heterocyclic group and aromatic group represented by R^{o} is optionally substituted with one or more independently selected groups represented by R^{#};
R^{#} is R⁺, —OR⁺, —O(haloalkyl), —SR⁺, —NO₂, —CN, —N(R⁺)₂, —NHCO₂R⁺, —NHC(O)R⁺, —NHNHC(O)R⁺, —NHC(O)N(R⁺)₂, —NNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂; and
R⁺ is —H, a C1-C3 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine; or —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

6. The compound of claim 4 wherein the compound is represented by the following structural formula: 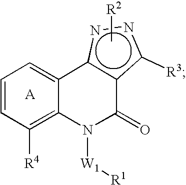
wherein Ring A is optionally substituted at any one or more substitutable ring carbon atoms.

7. The compound of claim 6 wherein:
R³ is methyl, ethyl, cyclopropyl, cyclopentyl, or tetrahydrofuryl; or
R³ is V₁—R^{3a}, wherein V₁ is a C1-C2 alkylidene and R^{3a} is —OH or —OCH₃.

8. The compound of claim 6 wherein:
R¹ is —CONR¹¹R¹², —COOR¹², an optionally substituted heteroaryl group or a non-aromatic heterocyclic group;
W₁ is —C(R²)₂—W₄—;
W₄ is a C1-C5 alkylidene group optionally substituted with —OH, —NH₂, C1-C3 alkylamine, C1-C3 dialkylamine, N-pyrrolidinyl, N-piperidinyl, N-morpholinyl, N-pyrazinyl, N′-acyl-N-pyrazinyl or N′-alkyl-N-pyrazinyl or with one or more methyl groups; and
each R²¹ is independently —H or —CH₃.

9. The compound of claim 8 wherein the compound is represented by the following structural formula: 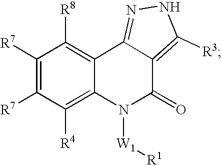
wherein:
R³ is —H, methyl, ethyl, n-propyl, iso-propyl, C1-C3 haloalkyl, C3-C6 cycloalkyl, tetrahydrofuryl or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
each R⁷ is independently —H, halogen, alkyl, haloalkyl, -T₁-V₃-R¹³, —NO₂, alkoxy, haloalkoxy or —CN;
R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃;
T, is a covalent bond, —O—, —NH—, —C(O)O—, —C(O)— or —C(O)NH—;
V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵ —NHC(O)OR¹⁴, —NHC(O)OR¹⁴, or a substituted or unsubstituted nitrogen-containing non-aromatic heterocyclic group wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group;
R¹³ is —CN, —OR¹⁴, —NR¹⁴R¹⁵, —C(O)NR¹⁴R¹⁵, —NHC(O)R¹⁴, —C(O)OR¹⁴, —NHC(O)NR¹⁴R¹⁵, —NHC(O)OR¹⁴, or an optionally substituted aromatic group or non-aromatic heterocyclic group; and
each R¹⁴ and each R¹⁵ is independently —H or C1-C3 alkyl or —NR¹⁴R¹⁵ is an optionally substituted non-aromatic heterocyclic group.

10. The compound of claim 9 wherein:
R³ is methyl, ethyl, cyclopropyl, cyclopentyl, or tetrahydrofuryl; or
R³ is V₁—R^{3a}, wherein V₁ is a C1-C2 alkylidene and R^{3a} is —OH or —OCH₃.

11. The compound of claim 9 wherein:
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
R⁴ and R⁸ are independently —H, halogen, —CH₃, halomethyl, —OCH₃, or haloalkoxy;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³;
R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and w is 0, 1 or 2.

12. The compound of claim 11 wherein W₁ is a C2-C5 alkylene group and R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen, —NO₂.

13. The compound of claim 6 wherein the compound is represented by the following structural formula: 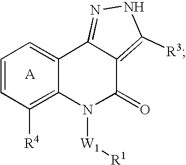
wherein Ring A is optionally substituted at any one or more substitutable ring carbon atoms.

14. The compound of claim 13 wherein:
R¹ is 2-piperidinyl, 3-piperidinyl, or 4-piperidinyl; and
W₁ is a C1-C3 alkylidene.

15. The compound of claim 14 wherein the compound is represented by the following structural formula: 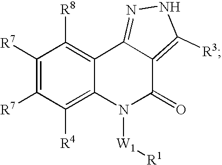
wherein:
R³ is —H, methyl, ethyl, n-propyl, iso-propyl, C1-C3 haloalkyl, C3-C6 cycloalkyl, tetrahydrofuryl or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
each R⁷is independently —H, halogen, alkyl, haloalkyl, -T₁-V₃—R¹³, —NO₂, alkoxy, haloalkoxy or —CN;
R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃;
T₁ is a covalent bond, —O—, —NH—, —C(O)O—, —C(O)— or —C(O)NH—;
V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵ —NHC(O)OR¹⁴, —NHC(O)OR¹⁴, or a substituted or unsubstituted nitrogen-containing non-aromatic heterocyclic group wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group;
R¹³ is —CN —OR¹⁴, —NR¹⁴R¹⁵, —C(O)NR¹⁴R¹⁵, —NHC(O)R¹⁴, —NHC(O)NR¹⁴R¹⁵, —NHC(O)OR¹⁴, —C(O)OR¹⁴ or an optionally substituted aromatic group or non-aromatic heterocyclic group; and
Each R¹⁴ and each R¹⁵ is independently —H or C1-C3 alkyl or —NR¹⁴R¹⁵ is an optionally substituted non-aromatic heterocyclic group.

16. The compound of claim 15 wherein:
R³ is —H, methyl, ethyl, cyclopropyl, cyclopentyl, or tetrahydrofuryl; or
R³ is V₁—R^{3a}, wherein V₁ is a C1-C2 alkylidene and R^{3a} is —OH, —OCH₃.

17. The compound of claim 15 wherein:
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
R⁴ and R⁸ are independently —H, halogen, —CH₃, halomethyl, —OCH₃, or haloalkoxy;
each R⁷ is independently —H, —Cl, —F, —Br, alkyl, —OH, alkoxy, haloalkyl, haloalkoxy, —C(O)NH₂, —C(O)NHCH₃, —C(O)N(CH₃)₂, —NH₂, —NHCH₃, —N(CH₃)₂, —NHC(O)H, —NHC(O)CH₃, —V₃—R¹³ or —O—V₃—R¹³;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen.

18. The compound of claim 13 wherein the compound is represented by the following structural formula: 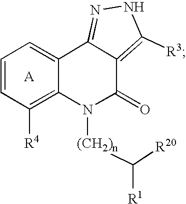
wherein:
R¹ is an optionally substituted nitrogen-containing heteroaryl group, an optionally substituted non-aromatic nitrogen-containing heterocyclic group, —COOR¹² or —CONR¹¹R¹²;
R¹¹ is —H and R¹² is cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, or —(CH₂)_{w}-pyrazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl;
R²⁰ is —OH, —NH₂, —CH₃, C1-C3 alkylamine, C1-C3 dialkylamine, N-pyrrolidinyl, N-piperidinyl, N-morpholinyl, N-pyrazinyl, N′-acyl-N-pyrazinyl or N′-alkyl-N-pyrazinyl;
w is 0, 1 or 2; and
n is an integer from 1 to 5.

19. The compound of claim 18 wherein the compound is represented by the following structural formula: 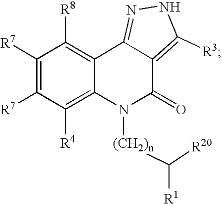
wherein:
R³ is —H, methyl, ethyl, n-propyl, iso-propyl, C1-C3 haloalkyl, C3-C6 cycloalkyl, tetrahydrofuryl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
each R⁷ is independently —H, halogen, alkyl, haloalkyl, -T₁-V₃—R¹³, —NO₂, alkoxy, haloalkoxy or —CN;
R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃;
T₁ is a covalent bond, —O—, —NH—, —C(O)O—, —C(O)— or —C(O)NH—;
V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵ —NHC(O)OR¹⁴, —NHC(O)OR¹⁴, or a substituted or unsubstituted nitrogen-containing non-aromatic heterocyclic group wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group;
R¹³ is —CN, —OR¹⁴, —NR¹⁴R¹⁵, —C(O)NR¹⁴R¹⁵, —NHC(O)R¹⁴, —NHC(O)NR¹⁴R¹⁵, —NHC(O)OR¹⁴, —C(O)OR¹⁴ or an optionally substituted aromatic group or non-aromatic heterocyclic group; and
each R¹⁴ and each R¹⁵ is independently —H or C1-C3 alkyl or —NR¹⁴R¹⁵ is an optionally substituted non-aromatic heterocyclic group.

20. The compound of claim 19 wherein:
R³ is methyl, ethyl cyclopropyl, cyclopentyl, tetrahydrofuryl; or
R³ is V₁—R^{3a}, wherein V₁ is a C1-C2 alkylidene and R^{3a} is —OH, —OCH₃.

21. The compound of claim 19 wherein:
R¹ is —CONR¹¹R¹²;
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R³a is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³;
R⁴ and R⁸ are independently —H, halogen, —CH₃, halomethyl, —OCH₃, haloalkoxy;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; and
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl.

22. The compound of claim 13 wherein:
R¹ is —OR¹², —NR¹¹R¹², —CN, an optionally substituted nitrogen-containing heteroaryl group, an optionally substituted non-aromatic nitrogen-containing heterocyclic group, —NHCOR¹², —NHCONR¹¹R¹², —OC(O)R¹²; NHC(O)OR¹², or —O—C(O)—NR¹¹R¹²;
W₁ is C2-C6 alkylene, —(CH₂)ₚ—CH(R²⁰)—CH₂—, —(CH₂)ₚ—C(R²¹)₂—CH₂— or —(CH₂)ₚ₊₁—C(R²¹)₂—;
R²⁰ is —OH, —OCH₃ —NH₂, —NHCH₃, —N(CH₃)₂ or —CH₃;
each R²¹ is —CH₃; and
p is an integer from 1 to 4.

23. The compound of claim 22 wherein the compound is represented by the following structural formula: 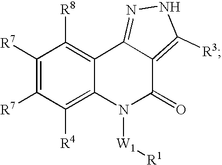
wherein:
R³ is —H, methyl, ethyl, n-propyl, iso-propyl, C1-C3 haloalkyl, C3-C6 cycloalkyl, tetrahydrofuryl or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃)₂, —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
each R⁷ is independently —H, halogen, alkyl, haloalkyl, -T₁-V₃—R¹³, —NO₂, alkoxy, haloalkoxy or —CN;
R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃;
T₁ is a covalent bond, —O—, —NH—, —C(O)O—, —C(O)— or —C(O)NH—;
V₃ is a covalent bond or a C1-C4 alkylidene, provided that V₃ is C2-C4 alkylidene when T₁ is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵ —NHC(O)OR¹⁴, —NHC(O)OR¹⁴, or a substituted or unsubstituted nitrogen-containing non-aromatic heterocyclic group wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group;
R¹³ is —CN, —OR¹⁴, —NR¹⁴R¹⁵, —C(O)NR¹⁴R¹⁵, —NHC(O)R¹⁴, —NHC(O)NR¹⁴R¹⁵, —NHC(O)OR¹⁴, —C(O)OR¹⁴ or an optionally substituted aromatic group or non-aromatic heterocyclic group; and
each R¹⁴ and each R¹⁵ is independently —H or C1-C3 alkyl or —NR¹⁴R¹⁵ is an optionally substituted non-aromatic heterocyclic group.

24. The compound of claim 23 wherein:
R³ is methyl, ethyl cyclopropyl, cyclopentyl, tetrahydrofuryl; or
R³ is V₁—R^{3a}, wherein V₁ is a C1-C2 alkylidene and R^{3a} is —OH, —OCH₃.

25. The compound of claim 23 wherein:
R¹ is —OH, —CN, —OR¹², —NH₂, —NR¹¹R¹², N-pyrrolidinyl, N-piperidinyl, N-morpholinyl, N-pyrazinyl, N′-acyl-N-pyrazinyl, N′-alkyl-N-pyrazinyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, N-tetrahydroquinolinyl or N-tetrahydroisoquinolinyl;
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
R⁴ and R⁸ are independently —H, halogen, —CH₃, halomethyl, —OCH₃, haloalkoxy;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³;
R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolidinyl, N-pyrrolidyl, N-alkyl-pyrrolidyl, N-acyl-pyrrolidyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and
w is 0, 1 or 2.

26. The compound of claim 25 wherein R¹ is —NH₂, —NHCH₃, —N(CH₃)₂, N-pyrazinyl, N′-methyl-N-pyrazinyl, N-morpholinyl, 2-piperidinyl or 3-piperidinyl; and W₁ is C2-C5 alkylene or —CH₂)ₚ—CH(CH₃)—CH₂—.

27. The compound of claim 23 wherein:
R¹ is —NHCONR¹¹R¹², —OC(O)R¹²; NHC(O)OR¹², or —O—C(O)—NR¹¹R¹²;
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³;
R⁴ and R³ are independently —H, halogen, —CH₃, halomethyl, —OCH₃, haloalkoxy;
R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-pyrrolidyl, N-piperidinyl, N-morpholinyl, imidazolyl, N-imizazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and
w is 0, 1 or 2.

28. The compound of claim 27 wherein R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen, or —NO₂.

29. The compound of claim 23 wherein:
R¹ is —NHCOR¹²;
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³;
R⁴ and R⁸ are independently —H, halogen, —CH₃, halomethyl, —OCH₃, haloalkoxy;
R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl or an optionally substituted —(CH₂)_{w}-aryl group;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-pyrrolidyl, N-piperidinyl, N-morpholinyl, imidazolyl, N-imizazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)N(CH₃)₂, —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and
w is 0, 1 or 2.

30. The compound of claim 29 wherein R¹² is alkyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl.

31. The compound of claim 13 wherein:
R¹ is —NR¹¹CO—CH(OR^{12a})—R¹², —NR¹¹CO—CH(NR^{12a}R^{12a})—R¹², —OC(O)—CH(OR^{12a})—R¹², —OC(O)—CH(NR^{12a}R^{12a})—R¹², —NR¹¹CO—C(R^{12c}R^{12c})—OR¹², —NR¹¹CO—C(R^{12c}R^{12c})—NR¹¹R¹², —OC(O)—C(R^{2c}R^{12c})—OR¹², —OC(O)—C(R^{12c}R^{12c})—NR¹¹R¹², —NHCO—CH(OH)—R¹², —NHCO—CH(NH₂)—R¹², —CH(OH)—CONR¹¹R¹², —CH(NH₂)—CONR¹², —OC(O)—CH(OH)—R¹², or —OC(O)—CH(NH₂)—R¹²;
W₁ is C2-C6 alkylene, —(CH₂)ₚ—CH(R²⁰)—CH₂—, —(CH₂)ₚ—C(R²¹)₂—CH₂— or —(CH₂)ₚ₊₁—C(R²¹)₂—;
R²⁰ is —OH, —OCH₃ —NH₂, —NHCH₃, —N(CH₃)₂ or —CH₃;
each R²¹ is —CH₃; and
p is an integer from 1 to 4.

32. The compound of claim 31 wherein: 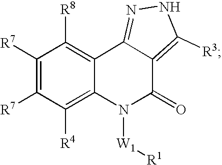
wherein:
R³ is —H, methyl, ethyl, n-propyl, iso-propyl, C1-C3 haloalkyl, C3-C6 cycloalkyl, tetrahydrofuryl or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
each R⁷ is independently —H, halogen, alkyl, haloalkyl, -T₁-V₃—R¹³, —NO₂, alkoxy, haloalkoxy or —CN;
R⁸ is —H, halogen, C1-C3 alkyl, C1-C3 haloalkyl, halogen, C1-C3 alkoxy, C1-C3 haloalkoxy, —NH₂, —NHCH₃, —N(CH₃)₂, —C(O)NH₂, —C(O)NHCH₃, —C(O)N(CH₃)₂, —NHC(O)H or —NHC(O)CH₃;
T₁ is a covalent bond, —O—, —NH—, —C(O)O—, —C(O)— or —C(O)NH—;
V₃ is a covalent bond or a C1-C4 alkylidene, provided that V₃ is C2-C4 alkylidene when T, is —O—, —NH—, —C(O)O—, or —C(O)NH— and R¹³ is —CN, —OH, —NR¹⁴R¹⁵, —NHC(O)R¹⁴, —OC(O)R¹², —NHC(O)NR¹⁴R¹⁵, —OC(O)NR¹⁴R¹⁵ —NHC(O)OR¹⁴, —NHC(O)OR¹⁴, or a substituted or unsubstituted nitrogen-containing non-aromatic heterocyclic group wherein a C1-C4 alkylidene group represented by V₃ is optionally substituted with a spirocyclopropyl group or one or two methyl groups and wherein a C1-C4 alkylidene group represented by V₃ is optionally fused to a cyclopropyl group;
R¹³ is —CN, —OR¹⁴, —NR¹⁴R¹⁵, —C(O)NR¹⁴R¹⁵, —NHC(O)R¹⁴, —NHC(O)NR¹⁴R¹⁵, —NHC(O)OR¹⁴, —C(O)OR¹⁴ or an optionally substituted aromatic group or non-aromatic heterocyclic group; and
each R¹⁴ and each R¹⁵ is independently —H or C1-C3 alkyl or —NR¹⁴R¹⁵ is an optionally substituted non-aromatic heterocyclic group.

33. The compound of claim 32 wherein:
R³ is methyl, ethyl cyclopropyl, cyclopentyl, tetrahydrofuryl; or
R³ is V₁—R^{3a}, wherein V₁ is a C1-C2 alkylidene and R^{3a} is —OH, —OCH₃.

34. The compound of claim 32 wherein:
R³ is —H, methyl, ethyl, n-propyl, iso-propyl, C1-C3 haloalkyl, or V₁—R^{3a}, wherein V₁ is a covalent bond or a C1-C2 alkylidene optionally substituted with one or two methyl groups or with a spiro cyclopropyl group; R^{3a} is —OH, —OCH₃, —NH₂, —NHCH₃, —N(CH₃)₂, —CONH₂, —CONHCH₃, —CON(CH₃)₂, —CN, —COOH, —COOCH₃, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl or N-morpholinyl;
R⁴ and R⁸ are independently —H, halogen, —CH₃, halomethyl, —OCH₃, or haloalkoxy;
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is—H, —Cl, —F, —Br, alkyl, haloalkyl, alkoxy, halomethoxy, —V₃—R¹³ or —O—V₃—R¹³;
R¹¹ is —H; and R¹² is alkyl, cyclopentyl, cyclohexyl, 2-aminocyclohexyl, 3-aminocyclohexyl, 4-aminocyclohexyl, 2-aminocyclopentyl, 3-aminocyclopentyl, 2-pyrrolidinyl, 2-piperidinyl, 2-morpholinyl, 3-pyrrolidinyl, 3-piperidinyl, 3-morpholinyl, 4-piperidinyl, tetrahydroquinolinyl, tetrahydroisoquinolinyl, tetrahydropyranyl, tetrahydrofuranyl, —(CH₂)_{w}-phenyl, —(CH₂)_{w}-pyrrolyl, —(CH₂)_{w}-pyrazolyl, —(CH₂)_{w}-imidazolyl, —(CH₂)_{w}-triazolyl, —(CH₂)_{w}-thiazolyl, —(CH₂)_{w}-isothiazolyl, —(CH₂)_{w}-oxazolyl, —(CH₂)_{w}-isoxazolyl, —(CH₂)_{w}-pyridyl, —(CH₂)_{w}-pyrimidinyl, —(CH₂)_{w}-pyrazinyl or —(CH₂)_{w}-triazinyl and wherein the —(CH₂)_{w}-phenyl or —(CH₂)_{w}-pyridyl group represented by R¹ is optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen; or —NR¹¹R¹² is dimethylamine, N-pyrrolidinyl, N-piperidinyl, N-morpholinyl, N-pyrazinyl, N′-acyl-N-pyrazinyl, N′-alkyl-N-pyrazinyl, N-tetrahydroquinolinyl or N-tetrahydroisoquinolinyl;
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-alkyl-piperazinyl, N-acyl-piperazinyl, pyrrolindyl, N-pyrrolidinyl, N-alkyl-pyrrolidinyl, N-acyl-pyrrolidinyl, piperidinyl, N-piperidinyl, N-alkyl-piperidinyl, N-acyl-piperidinyl or N-morpholinyl, imidazolyl, N-imidazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)N(CH₃)₂, —NHC(O)N(CH₃)₂, —NHC(O)OCH₃, halogen; N-piperazinyl, N′-alkyl-N-piperazinyl, N′-acyl-N-piperazinyl, N-pyrrolidyl, N-piperidinyl, or N-morpholinyl; and
w is 0, 1 or 2.

35. The compound of claim 34 wherein each R^{12a} is independently —H or —CH₃ or —NR^{12a}R^{12a} taken together is an aziridinyl group and each R^{12c} is —H, —CH₃ or —C(R^{12c}R^{12c})— taken together is a cyclopropyl group.

36. The compound of claim 35 wherein W₁ is a C2-C5 alkylene group and R¹² is alkyl, —(CH₂)_{w}-phenyl or —(CH₂)_{w}-pyridyl group, each optionally substituted with alkyl, haloalkyl, alkoxy, haloalkoxy, amine, alkylamine, dialkylamine, —C(O)NH₂, —C(O)NH(alkyl), —C(O)N(alkyl)₂, —NHC(O)H, —NHC(O)(alkyl), —CN, halogen, or —NO₂.

37. A compound represented by the following structural formula: 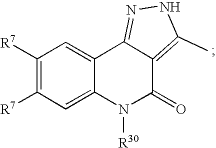
or a pharmaceutically acceptable salt thereof, wherein:
one R⁷ is —H, —Cl, —F, —Br, —CH₃, —OH, —OCH₃, halomethyl, halomethoxy, —C(O)NH₂, —C(O)NHCH₃, —C(O)N(CH₃)₂, —NH₂, —NHCH₃, —N(CH₃)₂, —NHC(O)H or —NHC(O)CH₃, and the other R⁷ is —H, —Cl, —F, —Br, alkyl, haloalkyl, alkoxy, halomethoxy, —V₃—R¹³ or —O—V₃—R¹³; and
R¹³ is —OH, —OCH₃, —CN, —NH₂, —NHCH₃, —N(CH₃)₂, —NHCH₂CH₃, —NH(CH₃)CH₂CH₃, —N(CH₂CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NHCH₃, —NHC(O)N(CH₃)₂, —NHC(O)OCH₃, piperazinyl, N-piperazinyl, N′-alkyl-N-piperazinyl, N′-acyl-N-piperazinyl, N-pyrrolidyl, N-piperidinyl, N-morpholinyl, imidazolyl, N-imizazolyl, pyrrolyl, N-pyrrolyl, pyridyl or phenyl optionally substituted with alkyl, —OH, —NH₂, —NHCH₃, —N(CH₃)₂, —C(O)NH₂, —C(O)NHCH₃, —C(O)N(CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)NH(CH₃), —NHC(O)N(CH₃)₂, —NHC(O)OCH₃, alkoxy, haloalkyl, haloalkoxy, —CN, NO₂ or halogen;
V₃ is a covalent bond or a C1-C4 unsubstituted alkylidene provided that V₃ is C2-C4 alkylidene when T₁ is —O—, and R¹³ is —OH, —CN, —NH₂, —NHCH₃, —N(CH₃)₂, —NHCH₂CH₃, —NH(CH₃)CH₂CH₃, —N(CH₂CH₃)₂, —NHC(O)H, —NHC(O)CH₃, —OC(O)H, —OC(O)CH₃, —OC(O)NH₂, —OC(O)NHCH₃, —OC(O)N(CH₃)₂, —NHC(O)NH₂, —NHC(O)N(CH₃)₂, —NHC(O)N(CH₃)₂, —NHC(O)OCH₃, N-piperazinyl, N′-alkyl-N-piperazinyl, N′-acyl-N-piperazinyl, N-pyrrolidyl, N-piperidinyl, or N-morpholinyl; and
R³⁰ is a structural formula selected from:

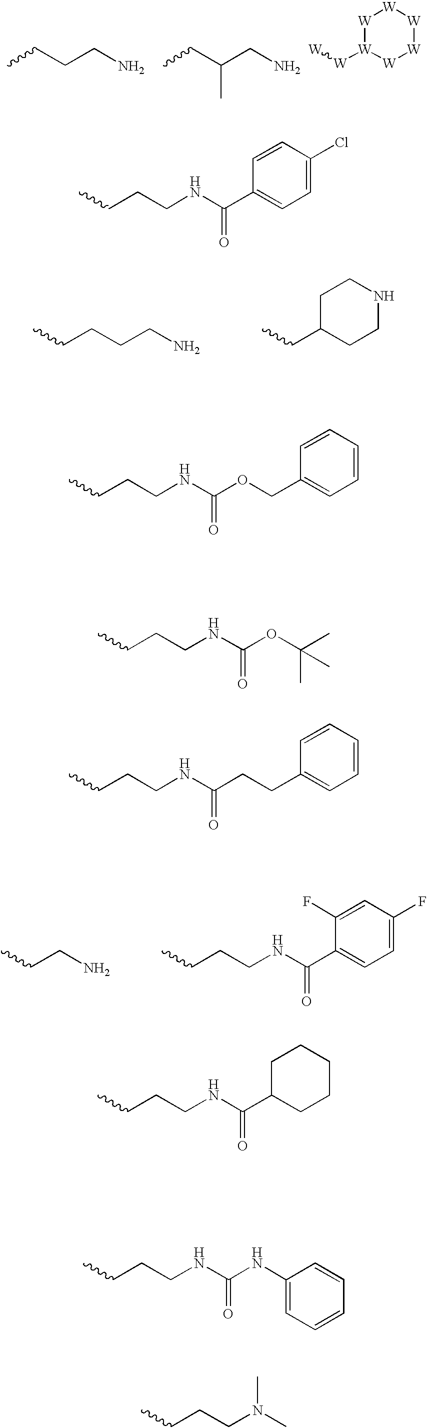

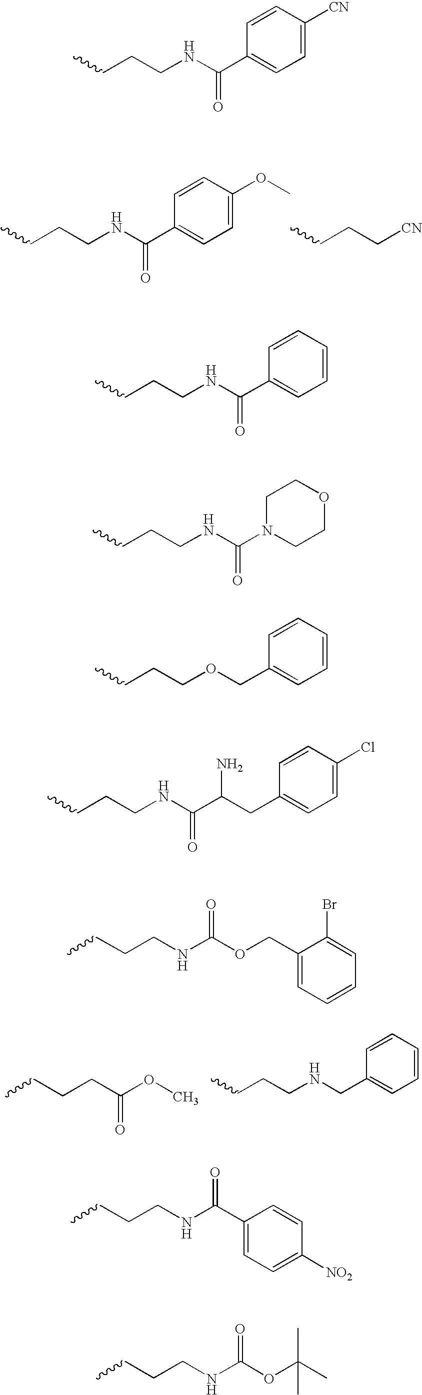

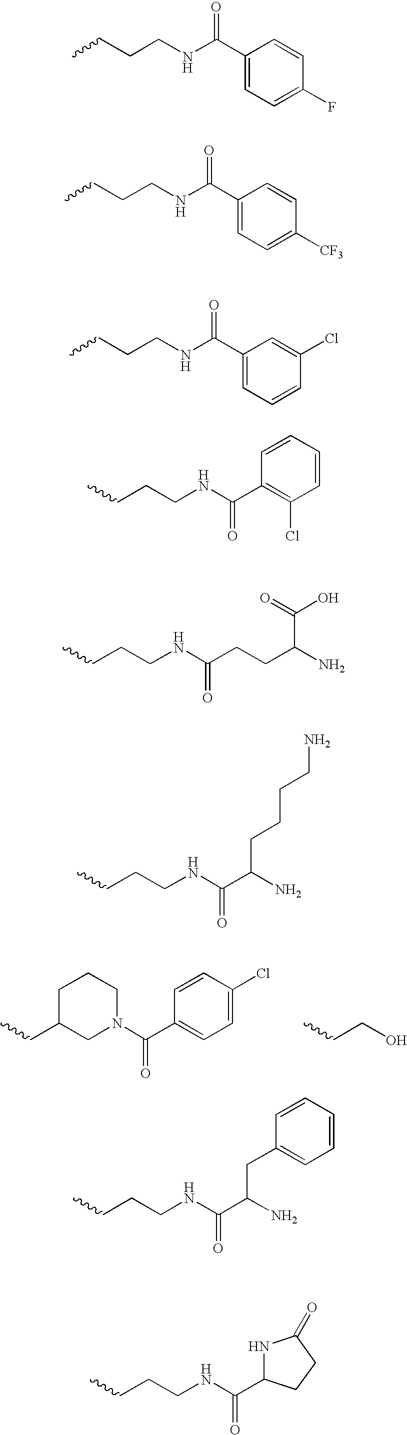

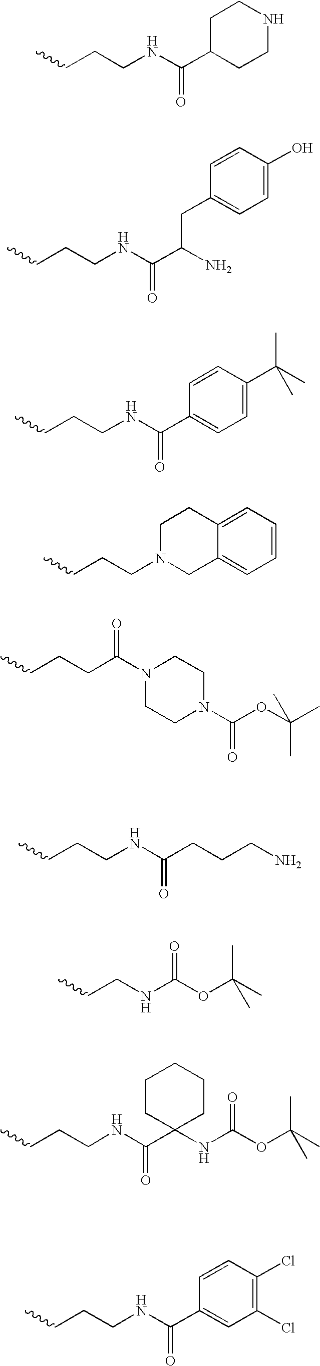

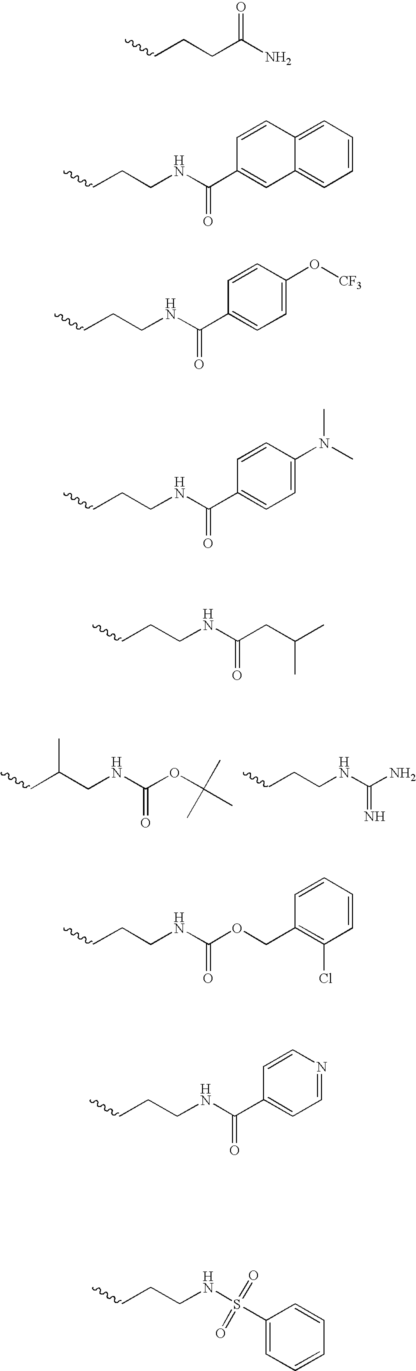

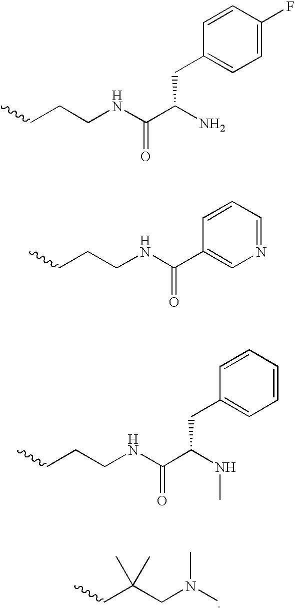

38. The compound of claim 3 wherein the compound is represented by the following structural formula: 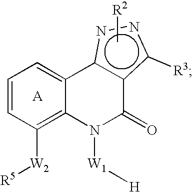
wherein:
Ring A is optionally substituted at any one or more substitutable ring carbon atoms.

39. A compound represented by the following structural formulas:

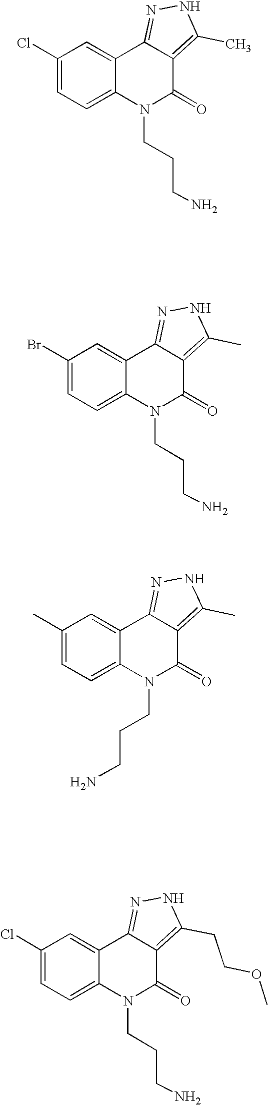

40. A method of treating a proliferative disorder in a subject comprising administering an effective amount of the Chk-1 inhibitor of claim 1.

41. The method of claim 42 wherein the proliferative disorder is a cancer.

42. The method of claim 41 wherein the cancer is one in which a checkpoint pathway has been mutated or upregulated.

43. The method of claim 42 wherein the Chk-1 inhibitor is administered in combination with another therapeutic agent.

44. The method of claim 43 wherein the Chk-1 inhibitor and the other therapeutic agent are administered as part of the same pharmaceutical composition.

45. The method of claim 44 wherein the Chk-1 inhibitor and the other therapeutic agent are administered as separate pharmaceutical compositions, and the Chk-1 inhibitor is administered prior to, at the same time as, or following administration of the other agent.

46. The method of claim 45 wherein the other therapeutic agent is an anticancer agent.

47. The method of claim 46 wherein the anticancer agent is selected from the group consisting of DNA damaging agents; cytotoxic agents; agents that disrupt cell replication; proteasome inhibitors; and NF-κB inhibitors.

48. The method of claim 47 wherein the anticancer agent is a DNA damaging agent.

49. The method of claim 48 wherein the DNA damaging agent is selected from the group consisting of radiation therapy, topoisomerase I inhibitors, topoisomerase II inhibitors, alkylating agents, DNA intercalators, and nucleoside mimetics.

50. A pharmaceutical composition comprising the compound of claim 1 and at least one pharmaceutically acceptable carrier or diluent.
